(19) United States
(12) Patent Application Publication
(10) Pub. No.: US 2023/0067929 A1
(43) Pub. Date: Mar. 2, 2023

(54) HETEROCYCLIC DERIVATIVES, PHARMACEUTICAL COMPOSITIONS AND THEIR USE IN THE TREATMENT, AMELIORATION OR PREVENTION OF CANCER

(71) Applicant: Tolremo Therapeutics AG, Zurich (CH)

(72) Inventors: Stefanie Flückiger-Mangual, Zurich (CH); Dorothea Gruber, Zurich (CH); Gerhard Müller, Utting am Ammersee (DE); Johan J.N. Veerman, Volendam (NL); Rutger Folmer, Wijchen (NL)

(73) Assignee: Tolremo Therapeutics AG, Zurich (CH)

(21) Appl. No.: 17/413,798

(22) PCT Filed: Dec. 17, 2019
(86) PCT No.: PCT/EP2019/085557

§ 371 Date: Jun. 14, 2021

(30) Foreign Application Priority Data
Dec. 17, 2018 (EP) 18213009.6
Oct. 2, 2019 (EP) 19201065.0

(51) Int. Cl. C07D 471/04; C07D 401/14; C07D 401/04; C07D 409/14; C07D 413/14; C07D 417/14; C07D 495/04; C07D 405/14; C07D 471/08; C07D 498/18

(52) U.S. Cl. CPC C07D 471/04; C07D 401/14; C07D 401/04; C07D 409/14; C07D 498/18; C07D 417/14; C07D 495/04; C07D 405/14; C07D 471/08; C07D 413/14

(57) ABSTRACT

The present invention relates to a compound of formula (I), optionally in the form of a pharmaceutically acceptable salt, solvate, cocrystal, tautomer, racemate, enantiomer, or diastereomer or mixture thereof Formula (I) and to pharmaceutical compositions comprising a compound of formula (I), as well as to the use of a compound of formula (I), or a pharmaceutically acceptable salt, solvate, cocrystal, tautomer, racemate, enantiomer, or diastereomer or mixture thereof, in the treatment of cancer. Further aspects of the present invention include combination therapies in which a compound of formula (I), as well as to the use of a compound of formula (I), or a pharmaceutically acceptable salt, solvate, cocrystal, tautomer, racemate, enantiomer, or diastereomer or mixture thereof, is used in combination with a known anti-cancer agent.

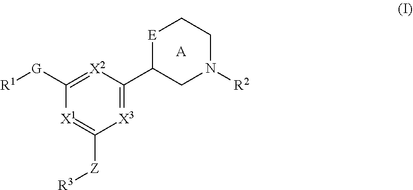

The present invention relates to a compound of formula (I), optionally in the form of a pharmaceutically acceptable salt, solvate, cocrystal, tautomer, racemate, enantiomer, or diastereomer or mixture thereof

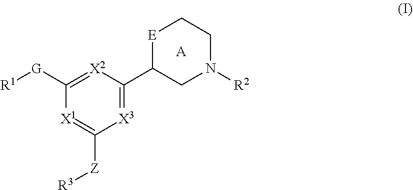

and to pharmaceutical compositions comprising a compound of formula (I), as well as to the use of a compound of formula (I), or a pharmaceutically acceptable salt, solvate, cocrystal, tautomer, racemate, enantiomer, or diastereomer or mixture thereof, in the treatment of cancer. Further aspects of the present invention include combination therapies in which a compound of formula (I), optionally in the form of a pharmaceutically acceptable salt, solvate, cocrystal, tautomer, racemate, enantiomer, or diastereomer or mixture thereof, is used in combination with a known anti-cancer agent.

### BACKGROUND OF THE INVENTION

Cancer is one of the most significant health conditions facing individuals in both developed and developing countries. It has been reported that in the United States alone, one in three people will be afflicted with cancer during their lifetime. Moreover, typically more than half of patients diagnosed with cancer eventually die as a result of the disease. Although significant progress has been made in the early detection and treatment of certain cancers, other cancers have been more difficult to detect and/or treat.

Furthermore, genetic alterations of cancer cells often affect genes that are important for cell cycle control, proliferation, differentiation and/or signal transduction. Oncogenic activation of MAPK pathway is a signature feature of many human cancers, including melanoma, non-small cell lung cancer (NSCLC) and pancreatic cancer. For example, 50% of melanomas are caused by the BRAF-V600E oncoprotein which activates constitutive MAPK signaling. BRAF-V600E specific small molecule inhibitors are the standard therapeutic approach for treatment of BRAF-V600E-positive metastatic melanoma. While this treatment leads to dramatic tumor shrinkage in the first few months, almost all patients acquire resistance as treatment continues. Clinical studies have shown that progression free survival can be extended by co-targeting BRAF and downstream kinase MEK but most patients still acquire resistance-causing genetic alterations limiting the benefit of these molecularly targeted drugs. Similar clinical hurdles are faced in the treatment of NSCLC with small molecule inhibitors or antibodies targeting EGFR as patients invariably acquire new, resistance causing mutations.

Phenotypic, signalling, transcriptional, and metabolic plasticity as well as the acquisition of novel genetic alterations have been found to be a driving factor in the development of resistance to cancer treatment including molecularly targeted inhibitors and immunotherapies. There is a need to avoid development of resistance to treatment.

Thus, an objective of the present invention is to provide novel compounds which are able to treat cancer or to prevent the development of resistance. Furthermore, it is an objective of the present invention to provide improved treatment options for cancer patients using the compounds of the invention alone or in combination therapy.

### BRIEF SUMMARY OF THE INVENTION

The present inventors have surprisingly found that compounds of the formula (I), optionally in the form of a pharmaceutically acceptable salt, solvate, cocrystal, tautomer, racemate, enantiomer, or diastereomer or mixture thereof

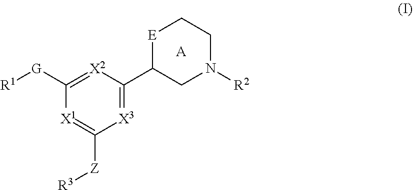

have activity against cancer.

The type of cancer that can be treated with the compounds and compositions of the present invention is not specifically limited and can be selected from non-melanoma skin cancer, esophagogastric adenocarcinoma, glioblastoma, bladder cancer, bladder urothelial carcinoma, esophagogastric cancer, melanoma, non-small cell lung cancer, endometrial cancer, cervical adenocarcinoma, esophageal squamous cell carcinoma, breast cancer, head and neck squamous cell carcinoma, germ cell tumor, small cell lung cancer, ovarian cancer, soft tissue sarcoma, hepatocellular carcinoma, colorectal adenocarcinoma, cervical squamous cell carcinoma, cholangiocarcinoma, prostate cancer, upper tract urothelial carcinoma, diffuse glioma, colorectal cancer, ampullary carcinoma, adrenocortical carcinoma, head and neck cancer, renal clear cell carcinoma, hepatobiliary cancer, glioma, non-Hodgkin lymphoma, mesothelioma, salivary gland cancer, renal non-clear cell carcinoma, miscellaneous neuroepithelial tumor, pheochromocytoma, thymic tumor, multiple myeloma, renal cell carcinoma, bone cancer, pancreatic cancer, leukemia, peripheral nervous system tumors, thyroid cancer, B-lymphoblast leukemia, monoclonal B-cell lymphocytosis, lymphoma, hairy cell leukemia, acute myeloid leukemia, Wilms tumor, in particular melanoma and non-small cell lung cancer.

### DESCRIPTION OF FIGURES

FIG. 1. POV-RAY drawing of a single 00209 molecule with the handedness of the chiral center indicated, based on the X-Ray data reported in the Examples.

FIG. 2: Powder X-ray diffractogram of bulk 00209.

FIG. 3: The initial Fo-Fc difference electron density map of the model (contoured at 4.0 σ) resulting from refinement of the initial model prior to modelling of the compound with REFMAC5, in the determination of the crystal structure of the bromodomain of human CREBBP in complex with compound 00212.

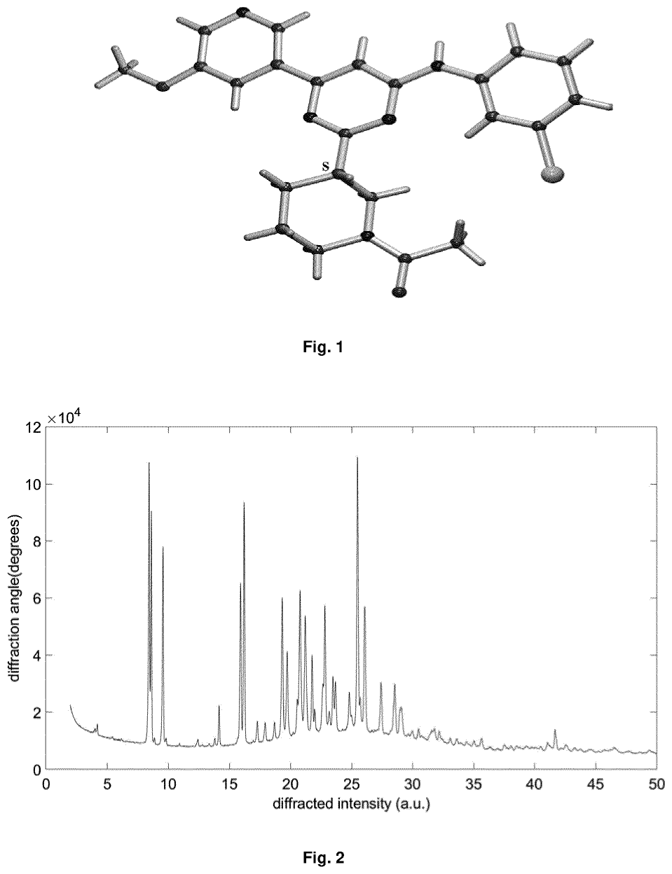

### DEFINITIONS

Unless defined otherwise, all technical and scientific terms used herein have the same meanings as commonly understood by one of ordinary skill in the art to which this invention belongs.

The term “preferably” is used to describe features or embodiments which are not required in the present invention but may lead to improved technical effects and are thus desirable but not essential.

The term “linked” in the expression “optionally linked” as used herein refers to a linked group which is obtained from two substituents by theoretically abstracting one hydrogen radical from each substituent and forming a single bond between the two radicals thus formed in the two substituents. This may be illustrated as follows:

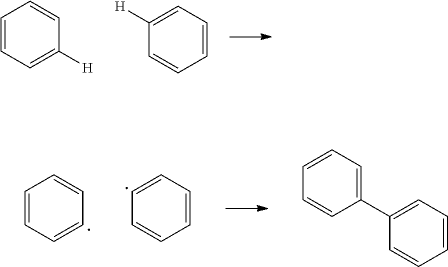

Although this explanation uses two aryl groups as an illustration, the meaning of the term “linked” is obviously not limited to such groups.

The term “hydrocarbon group which contains from 1 to 20 carbon atoms and optionally 1 to 15 heteroatoms selected from O, N and S” refers to any group having 1 to 20 carbon atoms and optionally 1 to 15 (preferably 1 to 10, more preferably 1 to 8) heteroatoms selected from O, N and S which preferably contains at least one ring. The “hydrocarbon group which contains from 1 to 20 carbon atoms and optionally 1 to 15 heteroatoms selected from O, N and S” is not limited in any way, provided that it is a group containing 1 to 20 carbon atoms and optionally 1 to 15 heteroatoms selected from O, N and S. E.g., if the hydrocarbon group is an aliphatic group, it may include one or more of the heteroatoms in the main chain or in one or more side chains. The term is also meant to include bicyclic, tricyclic and polycyclic versions thereof. If more than one ring is present, they can be separate from each other or be annelated. Examples of bicyclic hydrocarbon groups include fused bicyclic hydrocarbon groups such as naphthalene as well as linked hydrocarbon groups such as biphenyl, bridged bicyclic hydrocarbon groups such as 1,4-diazabicyclo[2.2.2]octane and spiro-type hydrogen groups. The ring(s) can be either carbocyclic or heterocyclic and can be saturated, unsaturated or aromatic. The carbon atoms and heteroatoms can either all be present in the one or more rings or some of the carbon atoms and/or heteroatoms can be present outside of the ring, e.g., in a linker group (such as —(CH₂)ₚ— with p=1 to 6). Examples of these groups include -(optionally substituted heterocyclyl) and -(optionally substituted carbocyclyl).

As used herein, the term “-(optionally substituted C₁₋₆ alkyl) which may contain one to three oxygen atoms between carbon atoms” preferably refers to a group in which one or more direct C—C bonds in the C₁₋₆ alkyl group are replaced by a C—O—C moiety. Examples thereof are —CH₂—CH₂—O—CH₃, —CH₂—CH₂—O—CH₂—CH₃, —CH₂—CH₂—O—CH₂—CH₂—O—CH₃ and —CH₂—CH₂—O—CH₂—CH₂—O—CH₂—CH₃.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkylene” refers to an alkanediyl group, i.e. a divalent saturated acyclic hydrocarbon group which may be linear or branched. A “C₁₋₆ alkylene” denotes an alkylene group having 1 to 6 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl. The number of carbon atoms in the carbocyclyl group is not particularly limited and is preferably 3 to 14, more preferably 3 to 7.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. Unless defined otherwise, “heterocyclyl” preferably refers to heteroaryl, heterocycloalkyl or heterocycloalkenyl. The number of carbon atoms in the carbocyclyl group is not particularly limited and is preferably 5 to 14, preferably 5 to 10.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), anthracenyl, or phenanthrenyl. Unless defined otherwise, an “aryl” preferably has 5 to 14 ring atoms, more preferably 5 to 10 ring atoms, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromenyl, xanthenyl, phenoxathiinyl, pyrrolyl (e.g., 2H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolizinyl, isoindolyl, indolyl (e.g., 3H-indolyl), indazolyl, purinyl, isoquinolyl, quinolyl, phthalazinyl, naphthyridinyl, quinoxalinyl, cinnolinyl, pteridinyl, carbazolyl, beta-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, furazanyl, phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzoxazolyl, benzisoxazolyl, benzimidazolyl, 1H-tetrazolyl, 2H-tetrazolyl, coumarinyl, or chromonyl. Unless defined otherwise, a “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, or adamantyl. Unless defined otherwise, “cycloalkyl” preferably refers to a C₃₋₁₄ cycloalkyl, and more preferably refers to a C₃₋₇ cycloalkyl. A particularly preferred “cycloalkyl” is a monocyclic saturated hydrocarbon ring having 3 to 7 ring members.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). “Heterocycloalkyl” may, e.g., refer to oxetanyl, tetrahydrofuranyl, piperidinyl, piperazinyl, aziridinyl, azetidinyl, pyrrolidinyl, imidazolidinyl, morpholinyl (e.g., morpholin-4-yl), pyrazolidinyl, tetrahydrothienyl, octahydroquinolinyl, octahydroisoquinolinyl, oxazolidinyl, isoxazolidinyl, azepanyl, diazepanyl, oxazepanyl or 2-oxa-5-aza-bicyclo[2.2.1]hept-5-yl. Unless defined otherwise, “heterocycloalkyl” preferably refers to a 3 to 14 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₄ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms and carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. “Heterocycloalkenyl” may, e.g., refer to 1,2,3,6-tetrahydropyridinyl. Unless defined otherwise, “heterocycloalkenyl” preferably refers to a 3 to 14 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. Very preferred “haloalkyl” as substituents for the inventive compounds are —CF₃, —CHF₂, and —CH₂—CF₃, and again further preferred are —CF₃ and —CHF₂.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

A skilled person will appreciate that the substituent groups comprised in the compounds of formula (I) may be attached to the remainder of the respective compound via a number of different positions of the corresponding specific substituent group. Unless defined otherwise, the preferred attachment positions for the various specific substituent groups are as illustrated in the examples.

As used herein, the term “about” preferably refers to ±10% of the indicated numerical value, more preferably to ±5% of the indicated numerical value, and in particular to the exact numerical value indicated.

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

A “solvate” refers to an association or complex of one or more solvent molecules and the compound of formula (I). Examples of solvents that form solvates include, but are not limited to, water, isopropanol, ethanol, methanol, dimethyl sulfoxide (DMSO), ethyl acetate, acetic acid, acetonitril, and ethanolamine. The term “hydrate” refers to the complex where the solvent molecule is water. It is to be understood that such solvates of the compounds of the formula (I) also include solvates of pharmaceutically acceptable salts of the compounds of the formula (I).

A “cocrystal” refers to a crystalline structure that contains at least two different compounds that are solid in their pure form under ambient conditions. Cocrystals are made from neutral molecular species, and all species remain neutral after crystallization; further, typically and preferably, they are crystalline homogeneous phase materials where two or more building compounds are present in a defined stoichiometric ratio. See hereto Wang Y and Chen A, 2013; and Springuel G R, et al., 2012; and U.S. Pat. No. 6,570,036.

Furthermore, the compounds of formula (I) may exist in the form of different isomers, in particular stereoisomers (including, e.g., geometric isomers (or cis/trans isomers), enantiomers and diastereomers) or tautomers. All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or 1H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

### DETAILED DESCRIPTION OF THE INVENTION

In a first aspect, the present invention provides a compound of formula (I), optionally in the form of a pharmaceutically acceptable salt, solvate, cocrystal, tautomer, racemate, enantiomer, or diastereomer or mixture thereof

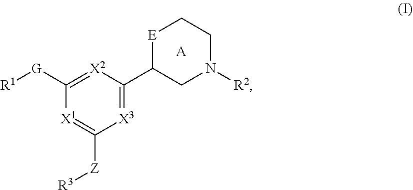

wherein
R¹ is selected from halogen and -(optionally substituted hydrocarbon group which contains from 1 to 20 carbon atoms and optionally 1 to 15 heteroatoms selected from O, N and S); R² is L-R²¹, wherein L is selected from a bond, —C(O)—, —C(O)—O—, —C(O)—NH—, —C(O)—N(C₁₋₆ alkyl)-, —S(O)— and —S(O)₂—; and
R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked;
each of X¹, X² and X³ is independently selected from N, CH and CR^{x};
Z is selected from —C(R³¹)₂—, —N(R³¹)— and —O—, wherein each R³¹ is independently selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—;
wherein Ring A may be substituted with one or more groups R^{x}, wherein any two R^{x} groups at ring A can be optionally linked and/or any R^{x} group at ring A can be optionally linked with R²;
wherein Ring A may furthermore be substituted to form a bicyclic moiety having the following partial structure:

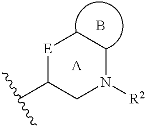

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with the proviso that the following compounds are excluded:

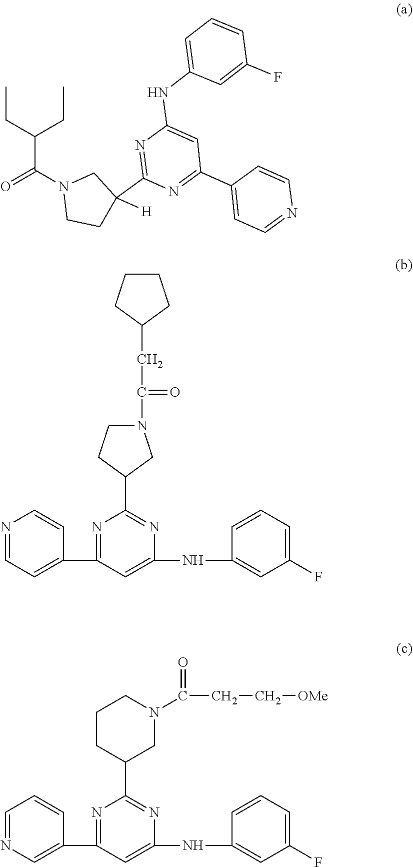 [SMILES: CCC(CC)C(=O)N1CCC(c2nc(Nc3cccc(F)c3)cc(-c3ccncc3)n2)C1 ; O=C(CC1CCCC1)N1CCC(c2nc(Nc3cccc(F)c3)cc(-c3ccncc3)n2)C1 ; COCCC(=O)N1CCCC(c2nc(Nc3cccc(F)c3)cc(-c3cccnc3)n2)C1]

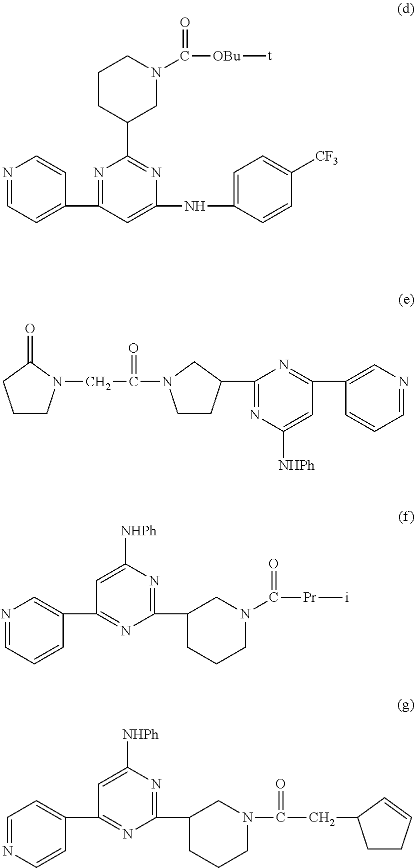

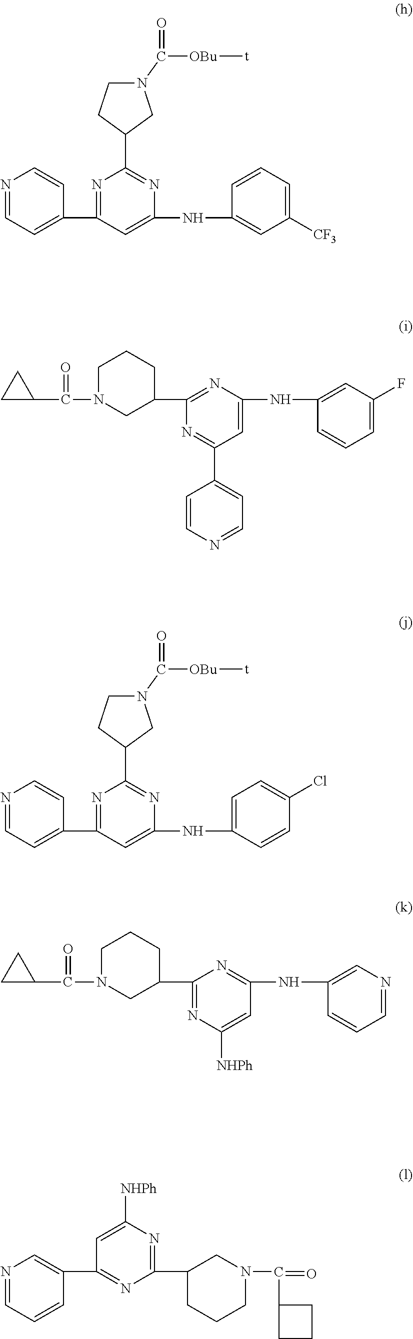

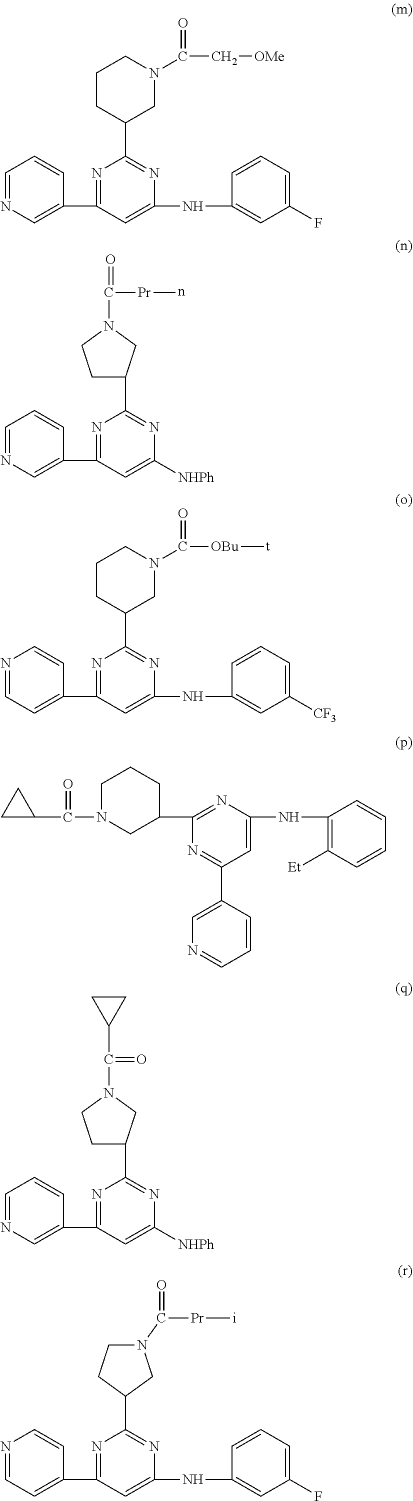

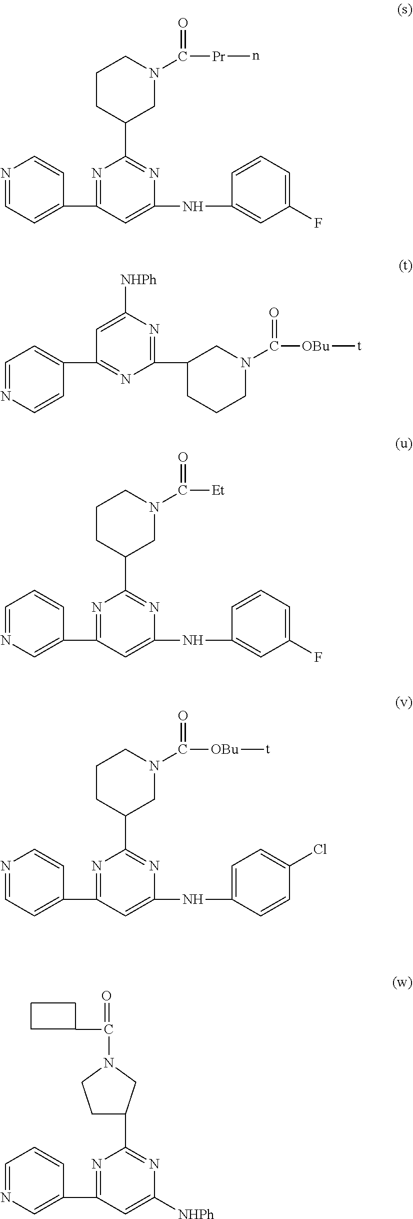

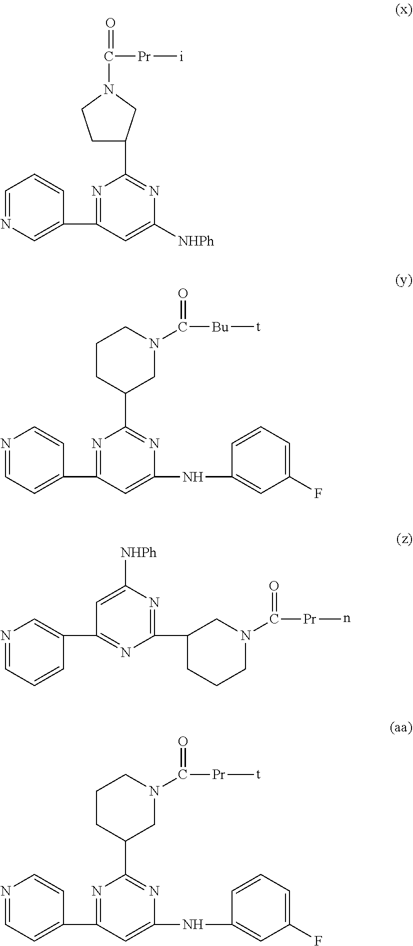

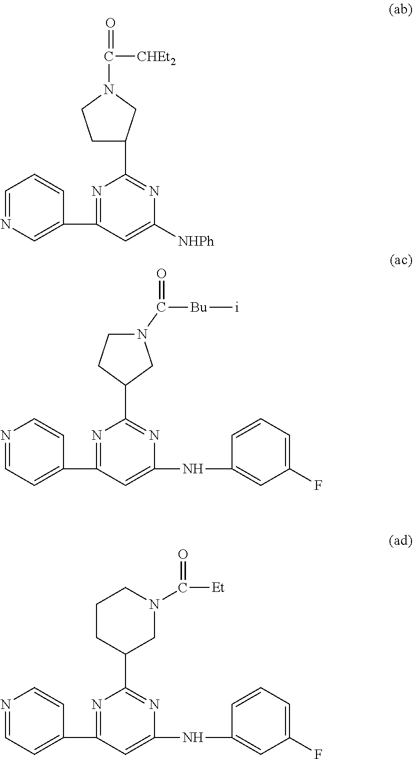

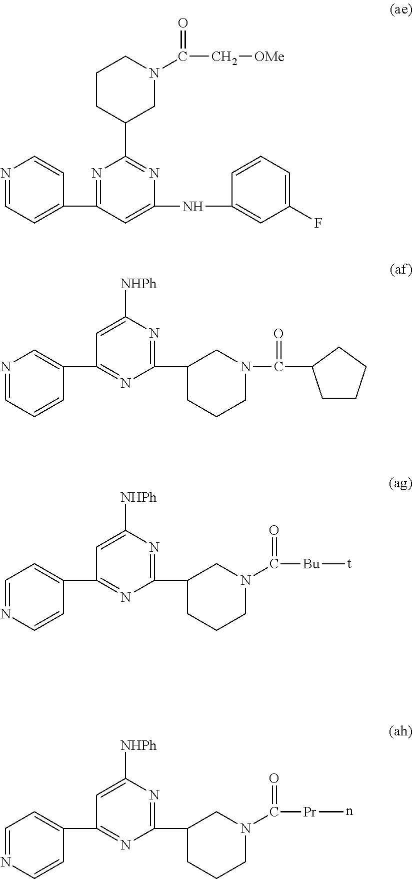

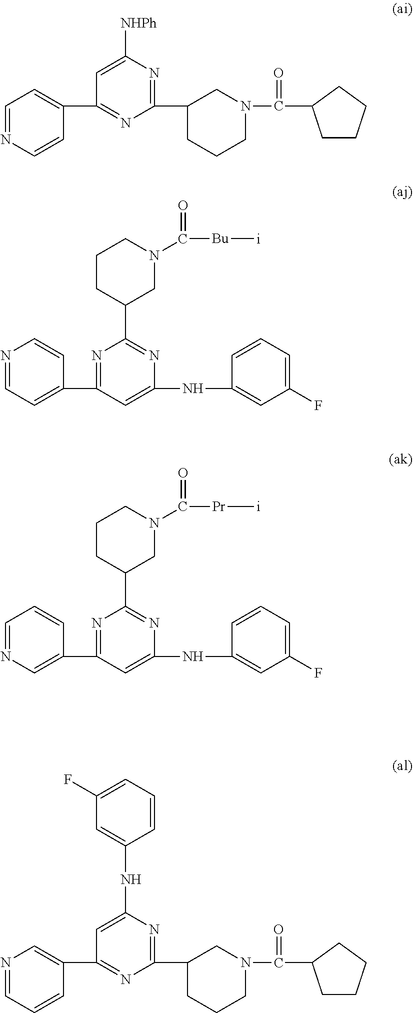

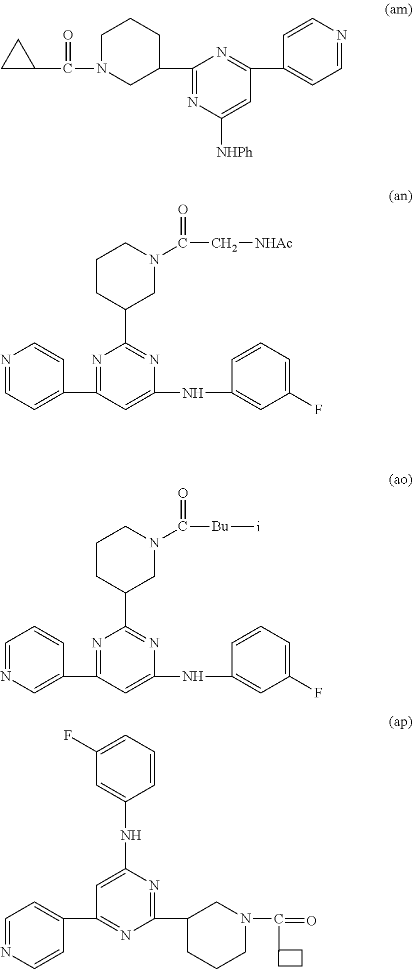

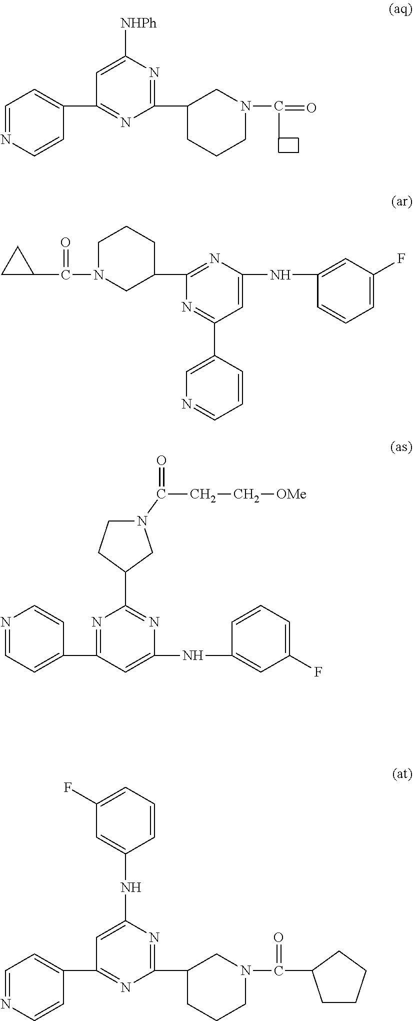

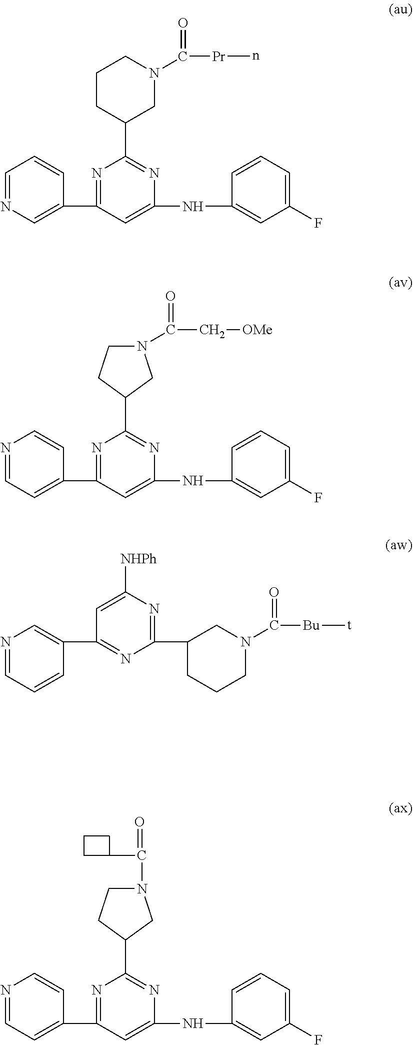

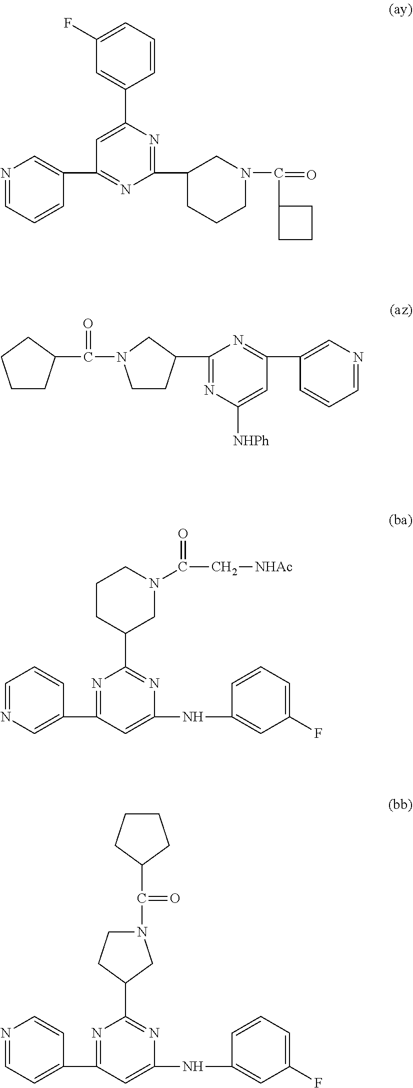

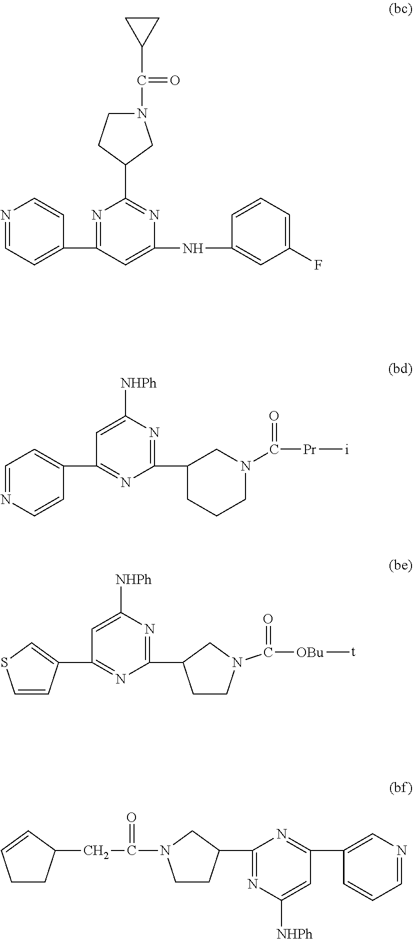

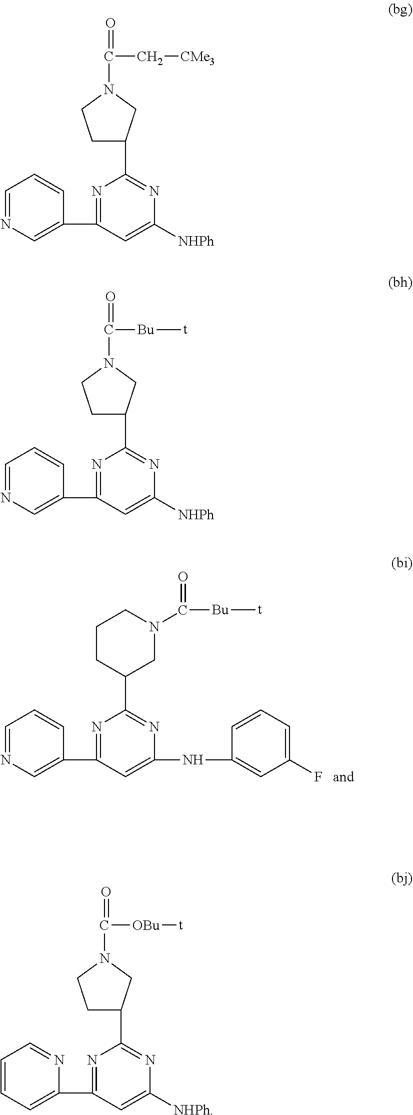

In a further aspect and embodiment, the present invention provides a compound of formula (I), optionally in the form of a pharmaceutically acceptable salt, solvate, cocrystal, tautomer, racemate, enantiomer, or diastereomer or mixture thereof

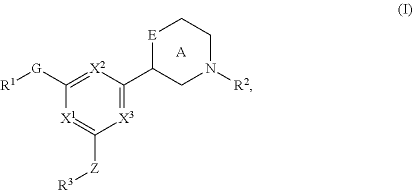

wherein
R¹ is selected from halogen and -(optionally substituted hydrocarbon group which contains from 1 to 20 carbon atoms and optionally 1 to 15 heteroatoms selected from O, N and S);
R² is L-R²¹, wherein L is selected from a bond, —C(O)—, —C(O)—O—, —C(O)—NH—, —C(O)—N(C₁₋₆ alkyl)-, —S(O)— and —S(O)₂—; and
R²¹ is selected from hydrogen, -(optionally substituted C₁₋₆ alkyl) which may contain one to three oxygen atoms between carbon atoms, and -(optionally substituted C₃₋₆ cycloalkyl); R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked;
each of X¹, X² and X³ is independently selected from N, CH and CR^{x};
Z is selected from —C(R³¹)₂—, —N(R³¹)— and —O—, wherein each R³¹ is independently selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}2-, —NH—, —NR^{x}—, —O—, -L¹-L²- and -L²-L¹-, wherein L¹ is selected from —CH₂—, —CHR^{x}—, —CR^{x}₂—, —NH—,
NR^{x}— and —O— and L² is selected from —CH₂—, —CHR^{x}— and —CR^{x}₂—;
wherein Ring A may be substituted with one or more groups R^{x}, wherein any two R^{x} groups at ring A can be optionally linked and/or any R^{x} group at ring A can be optionally linked with R²;
wherein Ring A may furthermore be substituted to form a bicyclic moiety having the following partial structure:

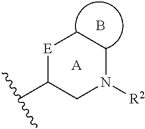

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and
wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with the proviso that the following compounds are excluded:

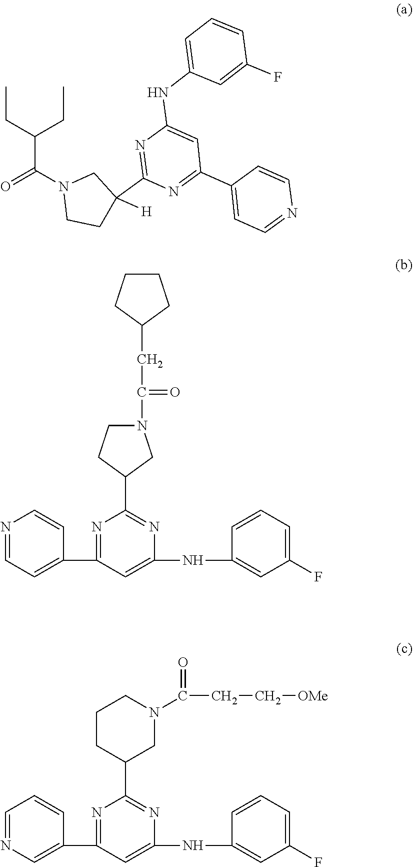 [SMILES: CCC(CC)C(=O)N1CCC(c2nc(Nc3cccc(F)c3)cc(-c3ccncc3)n2)C1 ; O=C(CC1CCCC1)N1CCC(c2nc(Nc3cccc(F)c3)cc(-c3ccncc3)n2)C1 ; COCCC(=O)N1CCCC(c2nc(Nc3cccc(F)c3)cc(-c3cccnc3)n2)C1]

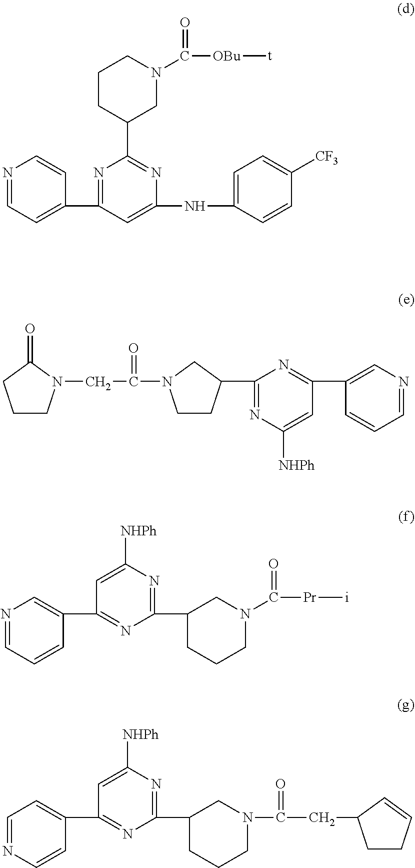

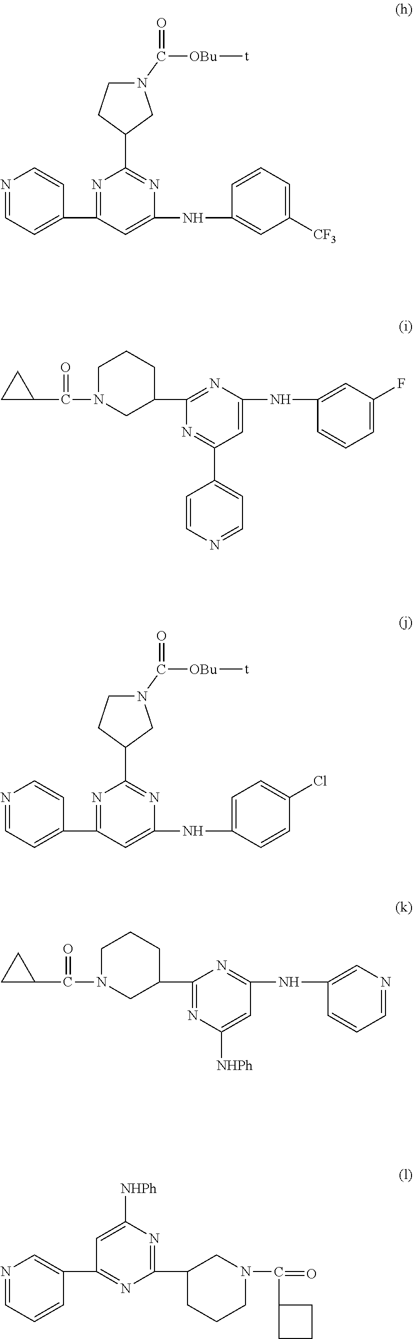

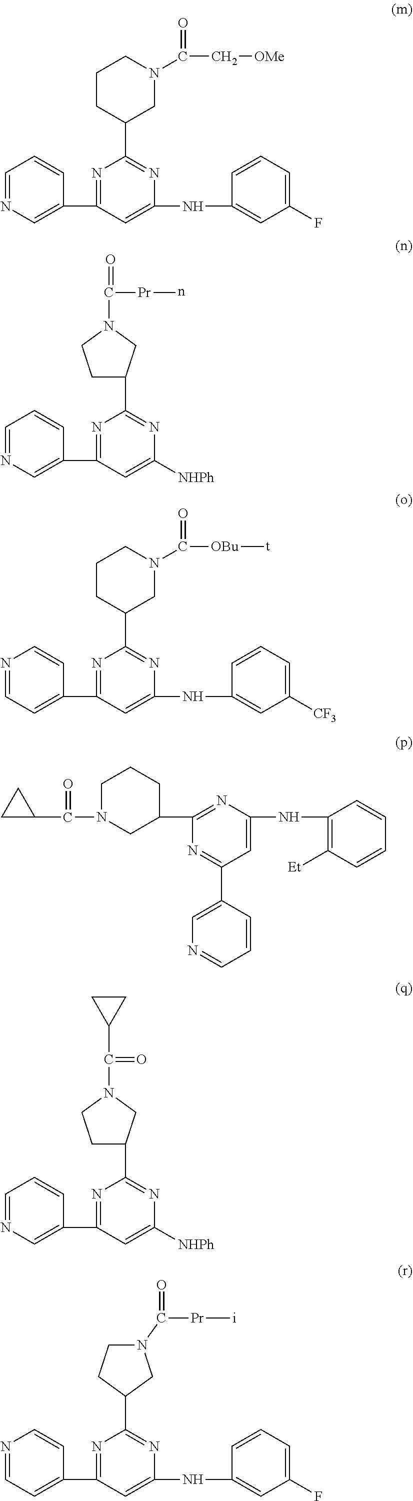

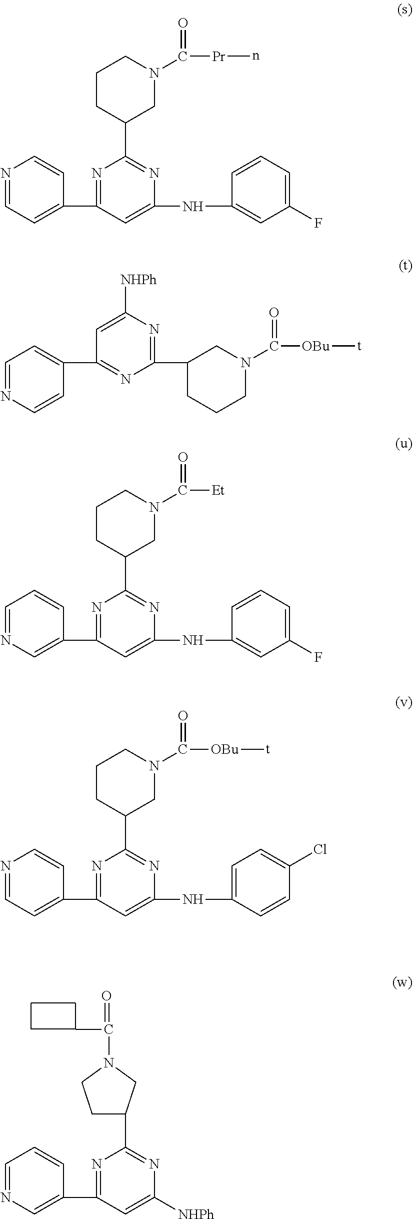

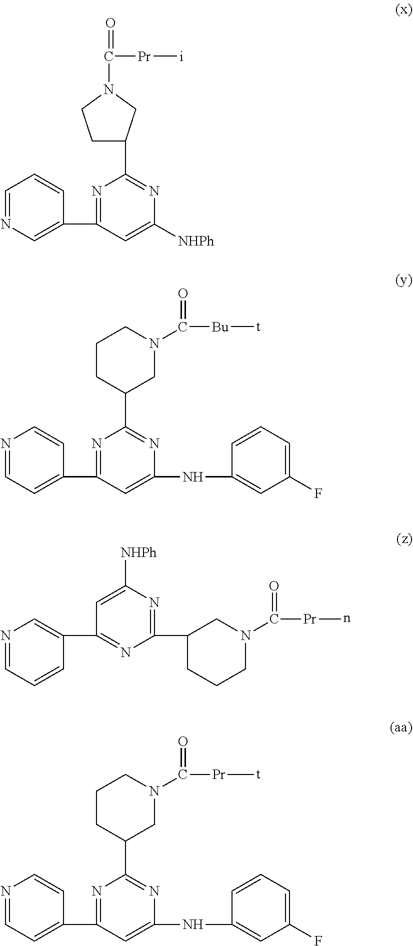

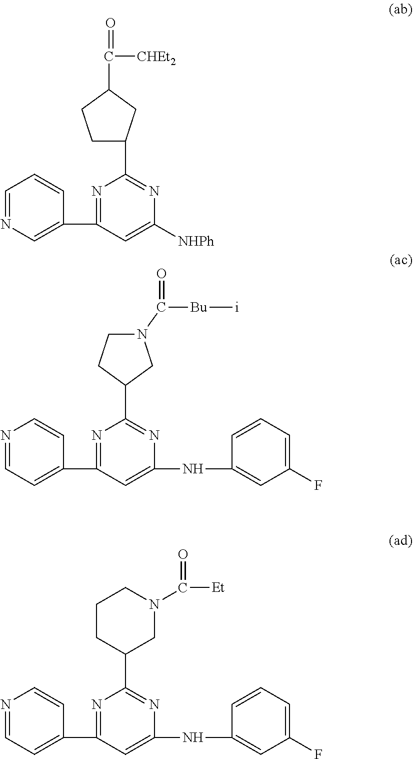

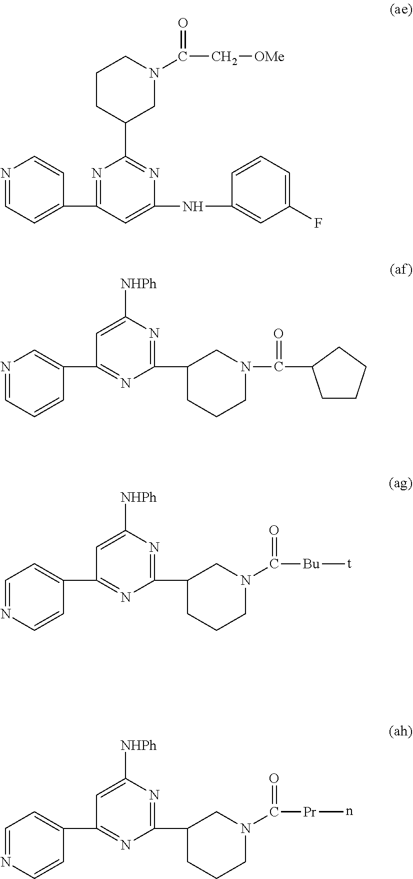

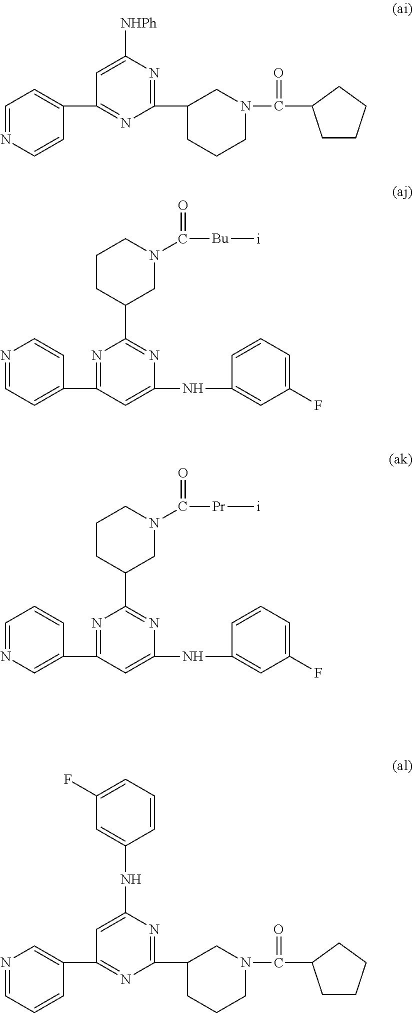

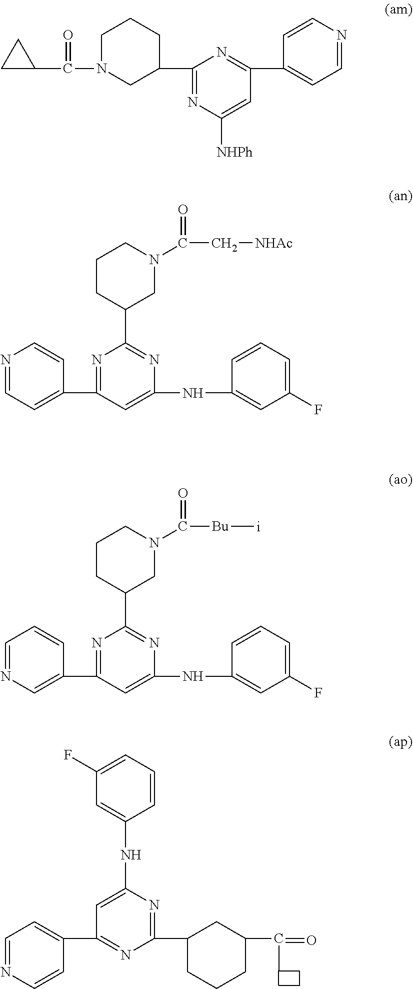

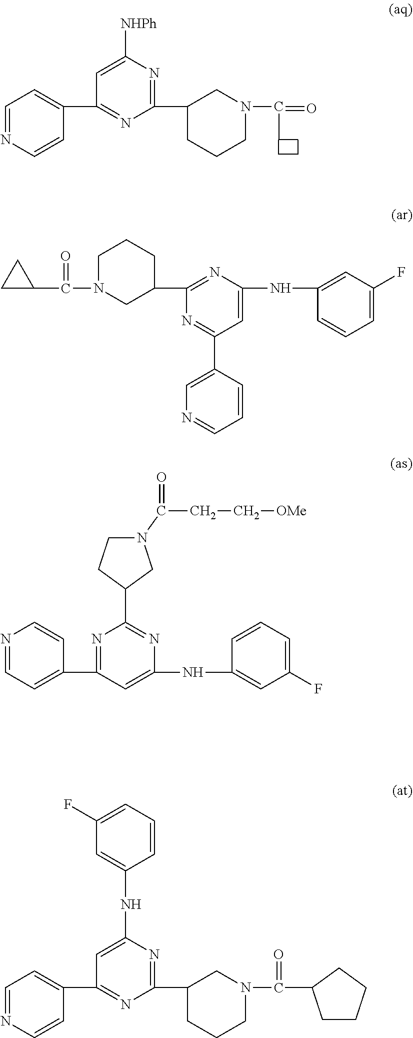

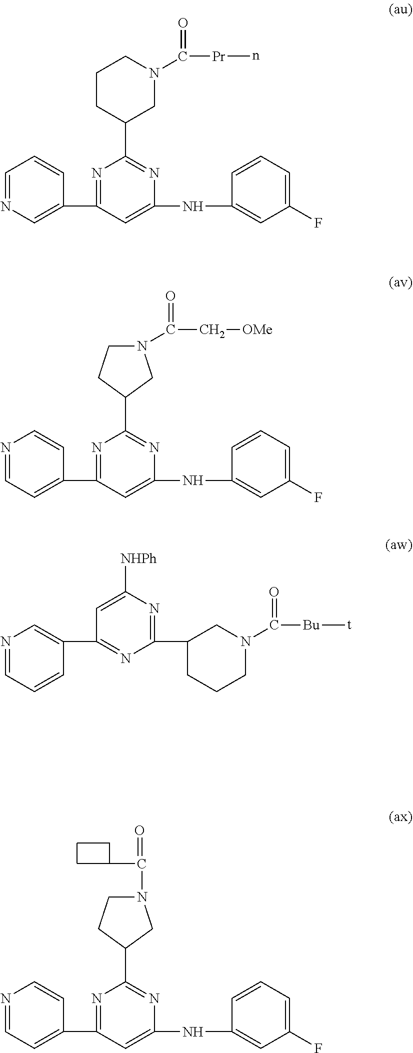

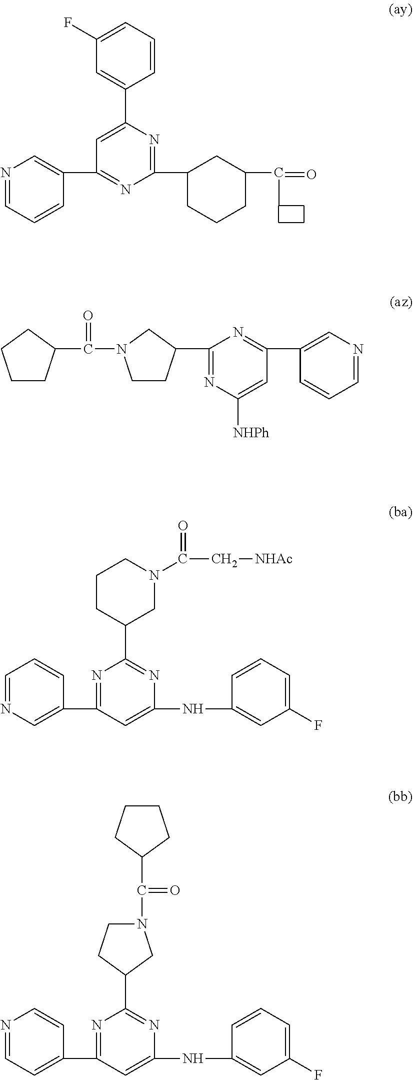

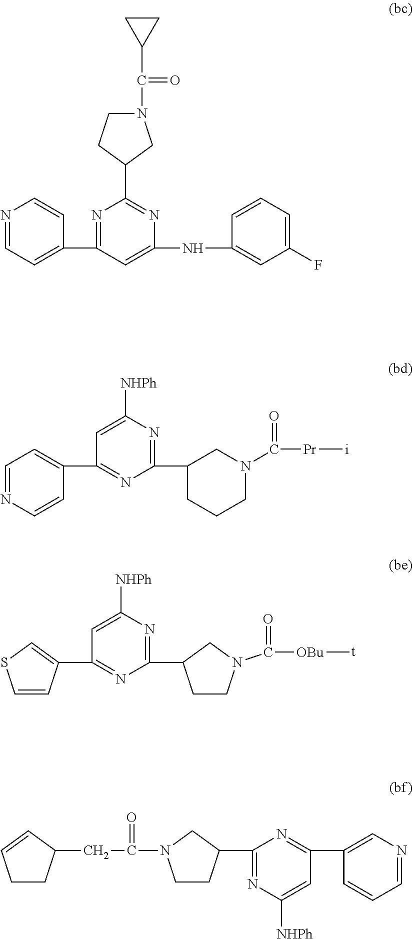

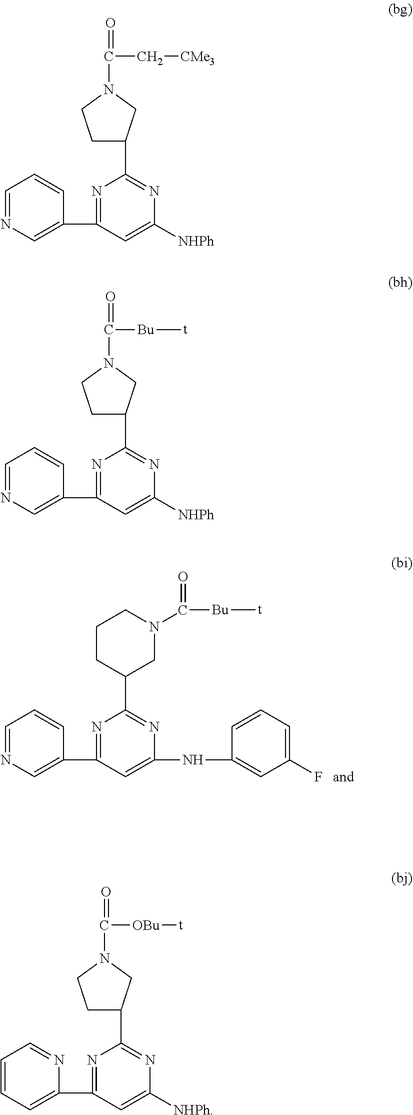

The present inventors have surprisingly found that compounds of formula (I), optionally in the form of a pharmaceutically acceptable salt, solvate, cocrystal, tautomer, racemate, enantiomer, or diastereomer or mixture thereof

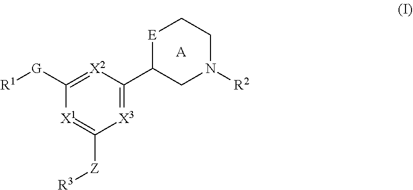

have improved activity.

In formula (I), the following definitions apply:

R¹ is selected from halogen and -(optionally substituted hydrocarbon group which contains from 1 to 20 carbon atoms and optionally 1 to 15 heteroatoms selected from O, N and S).

Preferably, R¹ is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

Further preferably, R¹ is selected from heterocyclyl which is substituted with -(optionally substituted heterocyclyl) or -(optionally substituted carbocyclyl).

More preferably, R¹ is phenyl, thiophenyl, pyridyl or pyrimidinyl, wherein the phenyl, thiophenyl, pyridyl or pyrimidinyl is optionally substituted with one or more substituents selected from halogen, —(C₁₋₆ alkyl which is optionally substituted with one or more halogen), —O—(C₁₋₆ alkyl which is optionally substituted with halogen), —C(O)—(C₁₋₆ alkyl which is optionally substituted with halogen), —NH—C(O)—(C₁₋₆ alkyl which is optionally substituted with halogen) and —C(O)—NH—(C₁₋₆ alkyl which is optionally substituted with halogen). Again more preferably, R¹ is phenyl, azaindolyl, azaindazolyl, pyrazinyl, thiophenyl, pyridyl or pyrimidinyl, wherein the phenyl, thiophenyl, pyridyl or pyrimidinyl is optionally substituted with one or more substituents selected from halogen, —(C₁₋₆ alkyl which is optionally substituted with one or more halogen), —O—(C₁₋₆ alkyl which is optionally substituted with halogen), —C(O)—(C₁₋₆ alkyl which is optionally substituted with halogen), —NH—C(O)—(C₁₋₆ alkyl which is optionally substituted with halogen) and —C(O)—NH—(C₁₋₆ alkyl which is optionally substituted with halogen).

R² is L-R²¹.

L is selected from a bond, —C(O)—, —C(O)—O—, —C(O)—NH—, —C(O)—N(C₁₋₆ alkyl)-, —S(O)— and —S(O)₂—, preferably L is —C(O)—. Preferably, L is selected from —C(O)—, —C(O)—O—, —C(O)—NH—, —C(O)—N(C₁₋₆ alkyl)-, —S(O)— and —S(O)₂—, preferably L is —C(O)—.

R²¹ is selected from -hydrogen, -(optionally substituted C₁₋₆ alkyl) which may contain one to three oxygen atoms between carbon atoms, and -(optionally substituted C₃₋₆ cycloalkyl). Preferably, R² is —C(O)-(optionally substituted C₁₋₆ alkyl).

R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl). Preferably, R³ is -(optionally substituted carbocyclyl). More preferably, R³ is phenyl which is optionally substituted with one or more groups selected from halogen, —(C₁₋₆ alkyl which is optionally substituted with one or more F) and —O—(C₁₋₆ alkyl which is optionally substituted with one or more F). Further preferred are compounds in which R³ is pyridinyl which may have the same substituents as the optionally substituted heterocyclyl. In other preferred compounds, R³ is quinazoline or cinnoline, each of which may have the same substituents as the optionally substituted heterocyclyl.

G is selected from a bond, —C(R¹¹)₂—, —N(R¹¹)— and —O—. Preferably, G is a bond.

Each R¹¹ is independently selected from -hydrogen, —C₁₋₆-alkyl, and —(C₁₋₆-alkyl substituted with one or more F) wherein R¹ and any R¹¹ can be optionally linked. When R¹ and an R¹¹ are linked, a cyclic group, such as a 3 to 8-membered ring containing 1 to 8 carbon atoms and optionally 1 to 4 heteroatoms selected from N, O and S may be formed. These cyclic groups typically include the carbon or nitrogen to which R¹¹ is bound as one ring member. Examples of such a cyclic group are cyclopentane, cyclohexane, pyrrolidine, piperidine and morpholine rings.

Each of X¹, X² and X³ is independently selected from N, CH and CRT Preferably, at least one of X² and X³ is N. More preferably, X¹ is nitrogen or CH, and X² and X³ are both N.

Z is selected from —C(R³¹)₂—, —N(R³¹)— and —O—. Preferably, Z is selected from —N(R³¹)— and —O—. More preferably, Z is —N(R³¹)—. Even more preferably, Z is —N(H)—.

Each R³¹ is independently selected from -hydrogen, —C₁₋₆-alkyl, and —(C₁₋₆-alkyl substituted with one or more F); wherein R³ and any R³¹ can be optionally linked. When R³ and an R³¹ are linked, a cyclic group, such as a 3 to 8-membered ring containing 1 to 8 carbon atoms and optionally 1 to 4 heteroatoms selected from N, O and S may be formed. These cyclic groups typically include the carbon or nitrogen to which R³¹ is bound as one ring member. Examples of such a cyclic group are cyclopentane, cyclohexane, pyrrolidine, piperidine and morpholine rings.

E is either absent or is selected from —CH₂—, —CHR^{x}—, —CR^{x}₂—, —NH—, —NR^{x}— and —O—. Alternatively, E may be selected from -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂— Preferably, E is selected from —CH₂—, —CHR^{x}—, —CR^{x}₂—, —NH—, —NR^{x}— and —O—. More preferably, E is selected from —CH₂—, —NH— and —O—. Still more preferably, E is CH₂ or O. Even more preferably, E is CH₂.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²; the number of groups R^{x} in Ring A is preferably 0, 1, or 2, more preferably 0 or 1.

Ring A may preferably be represented by a group represented by

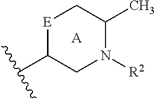

more preferably be represented by a group represented by

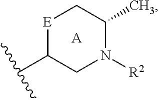

even more preferably

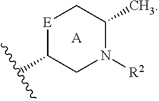

Ring A may furthermore be substituted to form a moiety having the following partial structure:

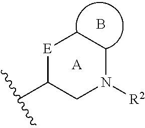

wherein Ring B is an -(optionally substituted heterocycle) or -(optionally substituted carbocycle). Preferably, Ring B is an optionally substituted aromatic monocyclic ring such as -(optionally substituted aryl) or -(optionally substituted heteroaryl) ring. Examples of Ring B include benzene, furan, thiophene, pyridine, pyrimidine, pyridazine, pyrazine, pyrrole, imidazole, pyrazole, isoxazole, isothiazole, oxazole, thiazole, oxadiazole, thiadiazole, triazole, tetrazole, each of which is optionally substituted. Examples of partial structures containing Rings A and B include:

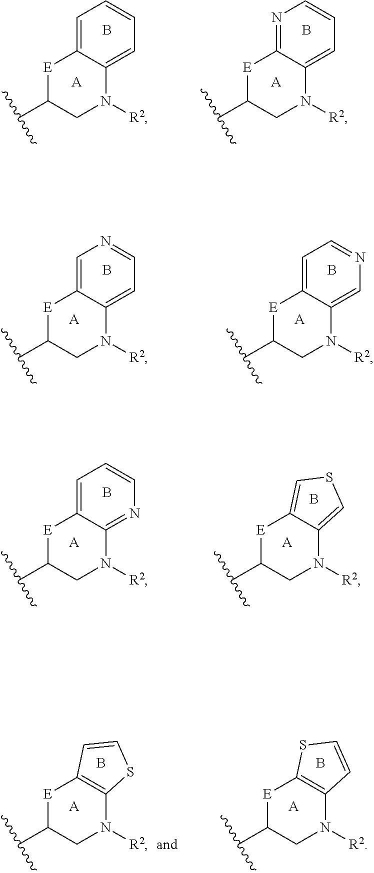

It is to be understood that Ring B in each of the above examples is optionally substituted. The optional substituent of Ring B is the same as the optional substituent of the -(optionally substituted heterocycle) or -(optionally substituted carbocycle).

Each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl). Further options of R^{x} include -(optionally substituted carbocyclyl) and -(optionally substituted heterocyclyl). Preferably, each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O and -(optionally substituted C₁₋₆ alkyl). More preferably, each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, =0 and -(optionally substituted C₁₋₆ alkyl).

The optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom are optionally linked, and

The optional substituent of the optionally substituted C₁₋₆ alkyl and the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom are optionally linked.

Preferred examples of the compound of formula (I) are compounds of formula (I-a)

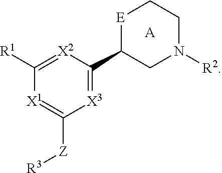

and compounds of formula (I-b)

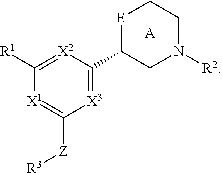

More preferred are the R-enantiomers of compounds of formula (I), i.e. compounds having the formula (I-b). The present inventors have surprisingly found that the R-enantiomers of the compounds of the present invention are significantly more active than the S-enantiomers.

Preferred examples of the compound of formula (I) are compounds of formula (II)

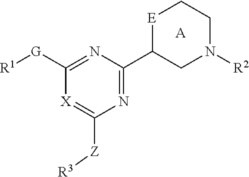

such as compounds of the following formula

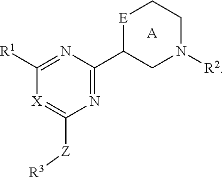

Preferred examples of the compound of formula (II) are compounds of formula (II-a)

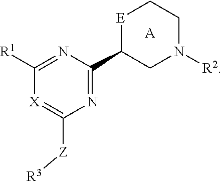

and compounds of formula (II-b)

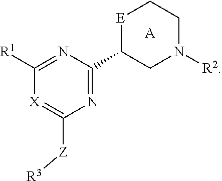

More preferred are the R-enantiomers of compounds of formula (I), i.e. compounds having the formula (II-b).

Even more preferred are R-enantiomers of compounds of the following formula (III):

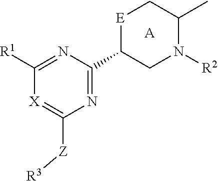

more preferably compounds of the following formula (III-a):

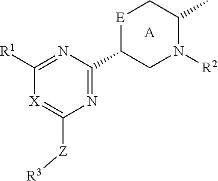

In the preferred examples of the compounds of formula (I), the definitions of the groups and substituents as specified with respect to formula (I) apply, unless explicitly mentioned otherwise. It is to be understood that X in the above structures has the same definitions as X¹ in the compounds of formula (i), namely it is independently selected from N, CH and CR^{x}.

In a further aspect and embodiment, the present invention provides a compound of formula (I), wherein said compound of formula (I) is a compound of formula (IV), preferably a compound of formula (IVa), optionally in the form of a pharmaceutically acceptable salt, solvate, cocrystal, tautomer, racemate, enantiomer, or diastereomer or mixture thereof

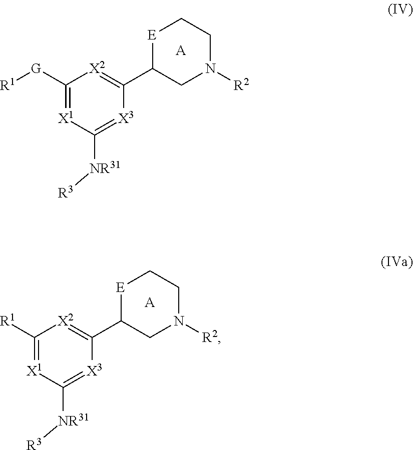

wherein
R¹ is selected from halogen and -(optionally substituted hydrocarbon group which contains from 1 to 20 carbon atoms and optionally 1 to 15 heteroatoms selected from O, N and S);
R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—;
wherein Ring A may be substituted with one or more groups R^{x}, wherein any two R^{x} groups at ring A can be optionally linked and/or any R^{x} group at ring A can be optionally linked with R²;
wherein Ring A may furthermore be substituted to form a bicyclic moiety having the following partial structure:

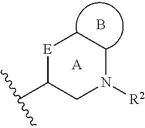

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and
wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N, preferably at least one of said X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CHR^{x}, —NH—, —NR^{x}— and —O— and L² is selected from —CH₂—, —CHR^{x}— and —CR^{x}₂— In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may furthermore be substituted to form a bicyclic moiety having the following partial structure:

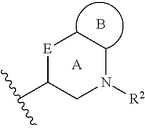

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁— alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (IVb), preferably a compound of formula (IVc), optionally in the form of a pharmaceutically acceptable salt, solvate, cocrystal, tautomer, racemate, enantiomer, or diastereomer or mixture thereof

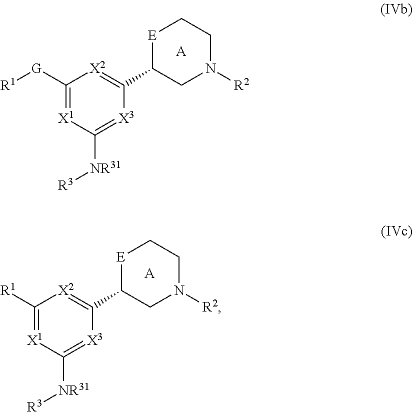

wherein
R¹ is selected from halogen and -(optionally substituted hydrocarbon group which contains from 1 to 20 carbon atoms and optionally 1 to 15 heteroatoms selected from O, N and S); R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—;
wherein Ring A may be substituted with one or more groups R^{x}, wherein any two R^{x} groups at ring A can be optionally linked and/or any R^{x} group at ring A can be optionally linked with R²;
wherein Ring A may furthermore be substituted to form a bicyclic moiety having the following partial structure:

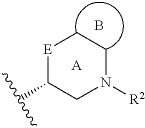

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may furthermore be substituted to form a bicyclic moiety having the following partial structure:

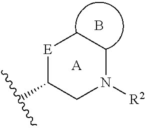

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (IVd′), preferably (IVd), or formula (IVe′), preferably (IVe), optionally in the form of a pharmaceutically acceptable salt, solvate, cocrystal, tautomer, racemate, enantiomer, or diastereomer or mixture thereof

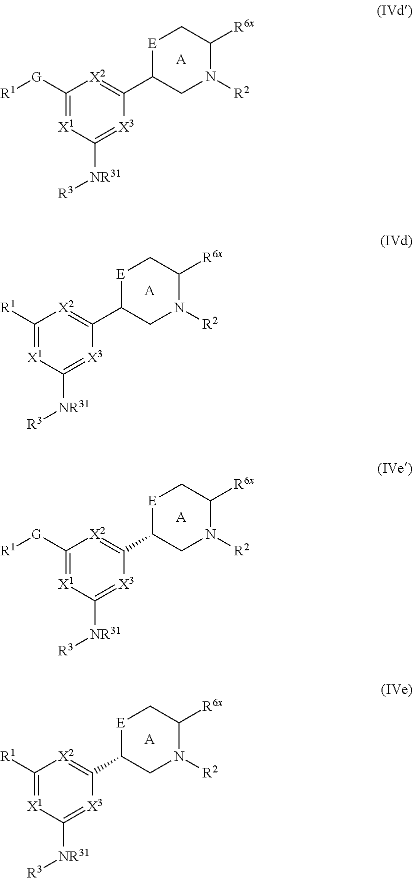

wherein
R¹ is selected from halogen and -(optionally substituted hydrocarbon group which contains from 1 to 20 carbon atoms and optionally 1 to 15 heteroatoms selected from O, N and S);
R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

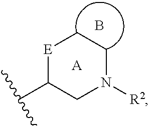

preferably

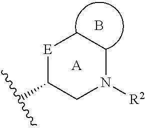

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

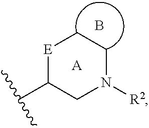

preferably

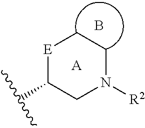

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound formula (IVf′), preferably (IVf), or formula (IVg′), preferably (IVg), optionally in the form of a pharmaceutically acceptable salt, solvate, cocrystal, tautomer, racemate, enantiomer, or diastereomer or mixture thereof

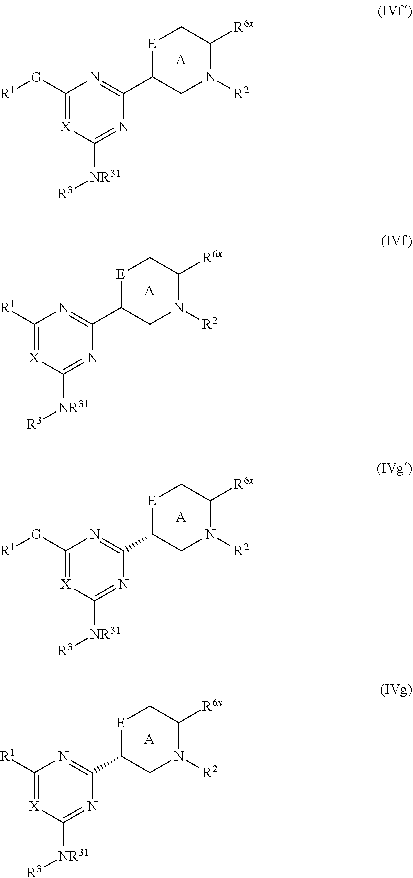

wherein
R¹ is selected from halogen and -(optionally substituted hydrocarbon group which contains from 1 to 20 carbon atoms and optionally 1 to 15 heteroatoms selected from O, N and S); R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
X is selected from N, CH and CR^{x}, preferably X is CH;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;

R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;

wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

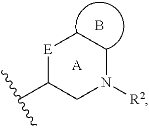

preferably

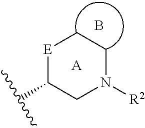

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, X is CH or N. In a further preferred embodiment, X is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH; In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

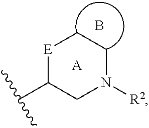

preferably

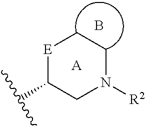

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (V), preferably a compound of (Va) optionally in the form of a pharmaceutically acceptable salt, solvate, cocrystal, tautomer, racemate, enantiomer, or diastereomer or mixture thereof

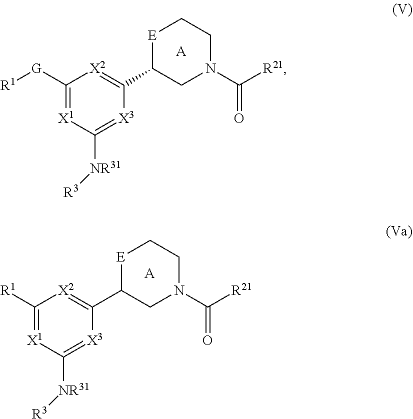

wherein
R¹ is selected from halogen and -(optionally substituted hydrocarbon group which contains from 1 to 20 carbon atoms and optionally 1 to 15 heteroatoms selected from O, N and S); R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

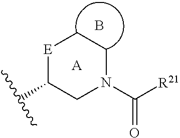

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and
wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

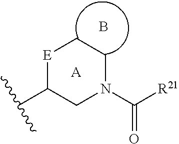

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₃ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (Vb), preferably a compound of (Vc) optionally in the form of a pharmaceutically acceptable salt, solvate, cocrystal, tautomer, racemate, enantiomer, or diastereomer or mixture thereof

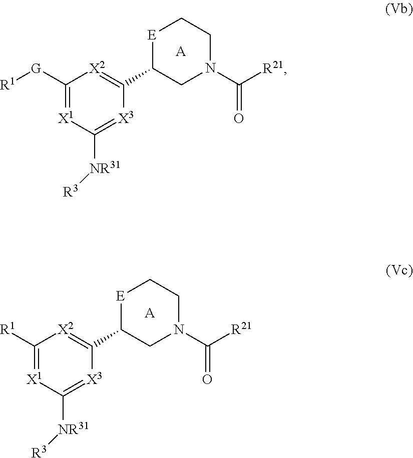

wherein
R¹ is selected from halogen and -(optionally substituted hydrocarbon group which contains from 1 to 20 carbon atoms and optionally 1 to 15 heteroatoms selected from O, N and S);
R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

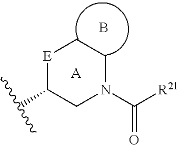

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

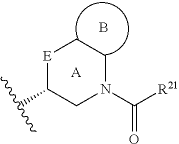

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₃ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (Vd′), preferably (Vd), or formula (Ve′), preferably (Ve), optionally in the form of a pharmaceutically acceptable salt, solvate, cocrystal, tautomer, racemate, enantiomer, or diastereomer or mixture thereof

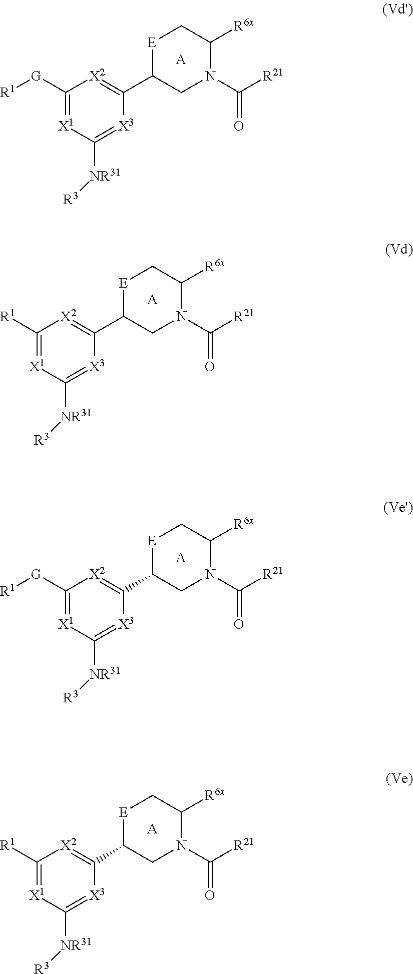

wherein
R¹ is selected from halogen and -(optionally substituted hydrocarbon group which contains from 1 to 20 carbon atoms and optionally 1 to 15 heteroatoms selected from O, N and S);
R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

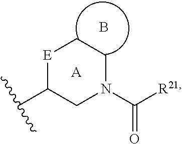

preferably

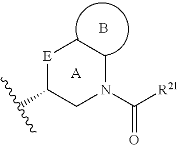

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and
wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

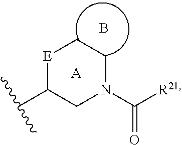

preferably

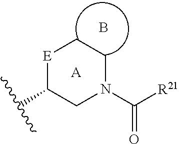

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (Vf′), preferably (Vf), or formula (Vg′), preferably (Vg), optionally in the form of a pharmaceutically acceptable salt, solvate, cocrystal, tautomer, racemate, enantiomer, or diastereomer or mixture thereof

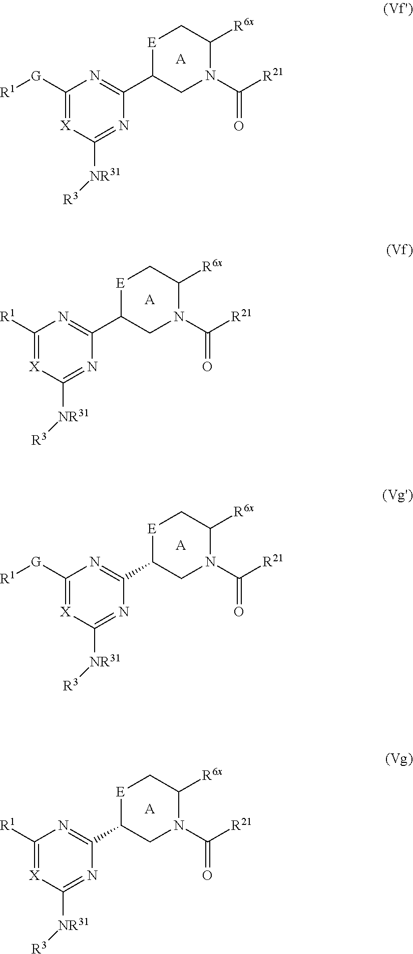

wherein
R¹ is selected from halogen and -(optionally substituted hydrocarbon group which contains from 1 to 20 carbon atoms and optionally 1 to 15 heteroatoms selected from O, N and S); R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
X is selected from N, CH and CR^{x}, preferably X is CH;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

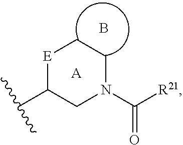

preferably

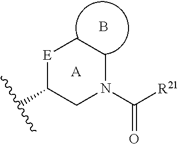

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, X is CH or N. In a further preferred embodiment, X is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, =0, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

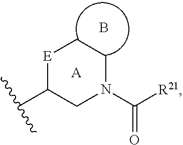

preferably

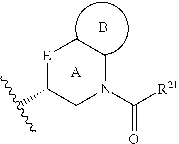

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (IVk), preferably of formula (IVm), optionally in the form of a pharmaceutically acceptable salt, solvate, cocrystal, tautomer, racemate, enantiomer, or diastereomer or mixture thereof

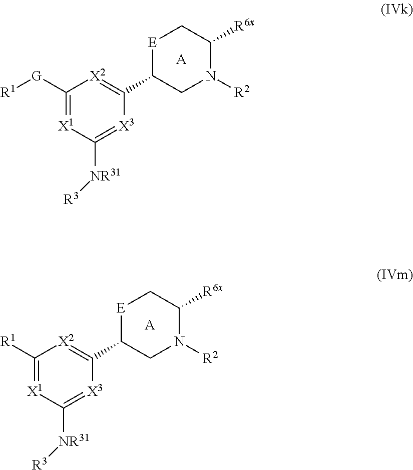

wherein
R¹ is selected from halogen and -(optionally substituted hydrocarbon group which contains from 1 to 20 carbon atoms and optionally 1 to 15 heteroatoms selected from O, N and S); R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl); R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

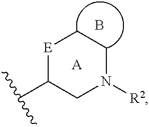

preferably

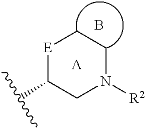

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CHR^{x}—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), -(optionally substituted carbocyclyl) and -(optionally substituted heterocyclyl), wherein said R^{xa} is independently selected from halogen, preferably-Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

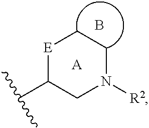

preferably

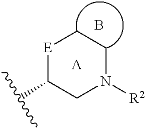

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (IVn), preferably (IVo), optionally in the form of a pharmaceutically acceptable salt, solvate, cocrystal, tautomer, racemate, enantiomer, or diastereomer or mixture thereof

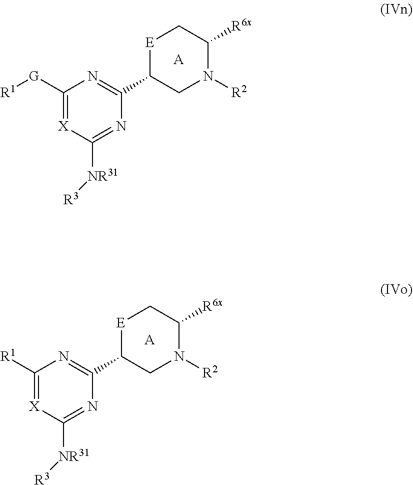

wherein
R¹ is selected from halogen and -(optionally substituted hydrocarbon group which contains from 1 to 20 carbon atoms and optionally 1 to 15 heteroatoms selected from O, N and S); R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any
R¹¹ can be optionally linked; preferably G is a bond;
X is selected from N, CH and CR^{x}, preferably X is CH;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

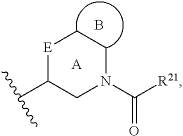

preferably

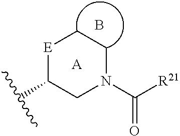

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and
wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked, with preferably the proviso that at least one, further preferably all of the compounds (a) to (bj) are excluded. In a further preferred embodiment, X is CH or N. In a further preferred embodiment, X is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), -(optionally substituted carbocyclyl) and -(optionally substituted heterocyclyl), wherein said R^{xa} is independently selected from halogen, preferably-Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

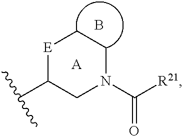

preferably

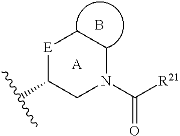

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (Vk), preferably (Vm), optionally in the form of a pharmaceutically acceptable salt, solvate, cocrystal, tautomer, racemate, enantiomer, or diastereomer or mixture thereof

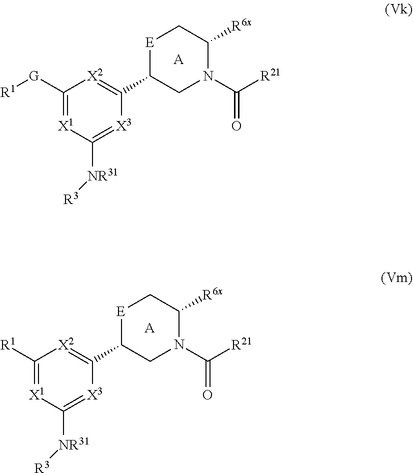

wherein
R¹ is selected from halogen and -(optionally substituted hydrocarbon group which contains from 1 to 20 carbon atoms and optionally 1 to 15 heteroatoms selected from O, N and S);
R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N; Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;

R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;

wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

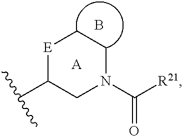

preferably

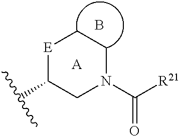

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), -(optionally substituted carbocyclyl) and -(optionally substituted heterocyclyl), wherein said R^{xa} is independently selected from halogen, preferably-Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

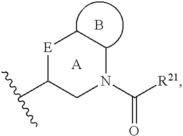

preferably

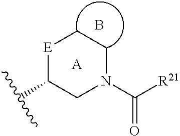

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl.

In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (Vn), preferably (Vo), optionally in the form of a pharmaceutically acceptable salt, solvate, cocrystal, tautomer, racemate, enantiomer, or diastereomer or mixture thereof

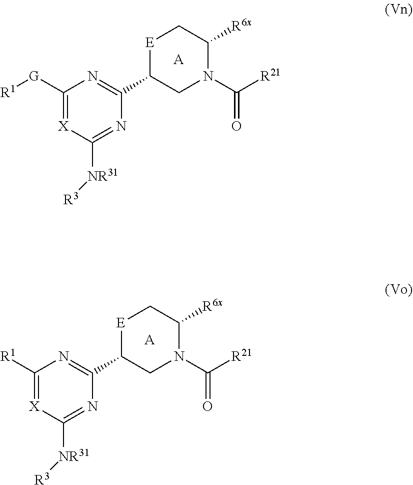

wherein
R¹ is selected from halogen and -(optionally substituted hydrocarbon group which contains from 1 to 20 carbon atoms and optionally 1 to 15 heteroatoms selected from O, N and S);
R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
X is selected from N, CH and CR^{x}, preferably X is CH;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with R^{6x} a bicyclic moiety having the following partial structure:

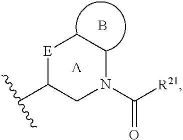

preferably

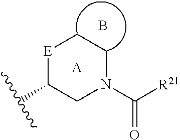

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, X is CH or N. In a further preferred embodiment, X is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), -(optionally substituted carbocyclyl) and -(optionally substituted heterocyclyl), wherein said R^{xa} is independently selected from halogen, preferably-Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with R^{6x} a bicyclic moiety having the following partial structure:

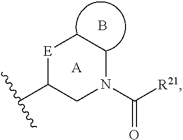

preferably

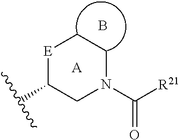

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, R^{6x} is selected from -halogen, —OH, ═O, C₁₋₄ alkyl, C₁₋₂ haloalkyl and C₁₋₃ alkyl substituted with one or more OH. In a further preferred embodiment, R^{6x} is selected from -halogen, —OH, ═O, C₁₋₃ alkyl, C₁₋₂ haloalkyl and C₁₋₃ alkyl substituted with one or two OH. In a further preferred embodiment, R^{6x} is selected from C₁₋₃ alkyl, C₁₋₂ haloalkyl and C₁₋₃ alkyl substituted with one or two OH. In a further preferred embodiment, R^{6x} is selected from C₁₋₂ alkyl, C₁₋₂ haloalkyl and C₁₋₃ alkyl substituted with one or two OH. In a further preferred embodiment, R^{6x} is selected from C₁₋₂ alkyl and C₁ haloalkyl. In a further preferred embodiment, R^{6x} is CHF₂. In a further preferred embodiment, R^{6x} is CF₃. In a further preferred embodiment, R^{6x} is ethyl. In a further very preferred embodiment, R^{6x} is methyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₃ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—Cu₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (VI), preferably (VIa), and further preferably (VIb), optionally in the form of a pharmaceutically acceptable salt, solvate, cocrystal, tautomer, racemate, enantiomer, or diastereomer or mixture thereof

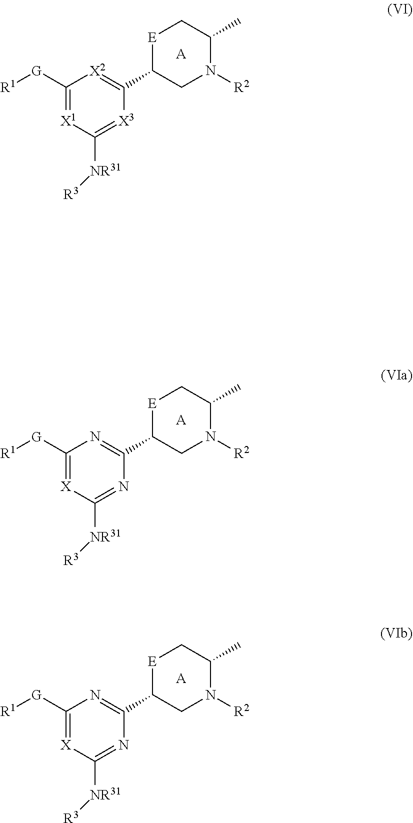

wherein
R¹ is selected from halogen and -(optionally substituted hydrocarbon group which contains from 1 to 20 carbon atoms and optionally 1 to 15 heteroatoms selected from O, N and S);
R² is L-R²¹, wherein L is selected from —C(O)—, —C(O)—O— and —C(O)—NH—; and R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N; alternatively X is selected from N, CH and CR^{x}, preferably X is CH;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with said methyl substitution group of Ring A a bicyclic moiety having the following partial structure:

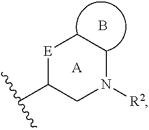

preferably

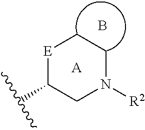

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with the methyl substitution group of Ring A a bicyclic moiety having the following partial structure:

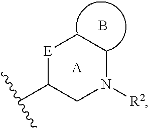

preferably

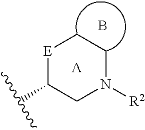

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

In a further preferred embodiment, said R¹-G- is selected from -(optionally substituted heterocyclyl), -(optionally substituted carbocyclyl), —O-(optionally substituted heterocyclyl), —O-(optionally substituted carbocyclyl), —NH-(optionally substituted heterocyclyl) and —NH-(optionally substituted carbocyclyl).

In a further preferred embodiment, G is absent and R¹— is selected from -(optionally substituted heterocyclyl) and -(optionally substituted carbocyclyl).

In a further preferred embodiment, G is absent and R¹— is selected from -(optionally substituted heteroaryl) and -(optionally substituted aryl), and wherein said, preferably one or two, optional substituent of said heteroaryl or said phenyl is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked.

In a further preferred embodiment, G is absent and R¹— is selected from -(optionally substituted heteroaryl) and -(optionally substituted phenyl), wherein said heteroaryl is a 5 or 6 membered monocyclic ring or 10 to 12 membered fused ring system comprising one or more ring heteroatoms independently selected from O, S and N, wherein one or two carbon ring atoms are optionally oxidized, and wherein said, preferably one or two, optional substituent of said heteroaryl or said phenyl is independently selected from —C₁₋₆ alkyl, C₁₋₆ haloalkyl, -halogen, —CN, ═O, —C(O)R*, —COOR*, —C(O)NR*R*, —NR*R*, —N(R*)—C(O)R*, —N(R*)—C(O)—OR*, —N(R*)—C(O)—NR*R*, —O—C(O)R*, —O—C(O)—NR*R*, —OR*; and carbocyclyl and heterocyclyl, each independently optionally substituted with, preferably one or two, halogen or C₁₋₄ alkyl; wherein each R* is independently selected from H, C₁₋₄ alkyl, C₁₋₄ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

In a further preferred embodiment, G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—C₁₋₃ haloalkyl.

In a further preferred embodiment, R¹ is 3-pyridyl or 3-pyridyl substituted at the meta position (5 position) with one substituent selected from halogen, —OH, —C₁₋₃ alkyl, C₁₋₂ haloalkyl, —O—(C₁₋₃ alkyl), —O—(C₁₋₂ haloalkyl), —C(O)—C₁₋₃ alkyl, —C(O)—C₁₋₂ haloalkyl, —NH—C(O)—C₁₋₃ alkyl, —NH—C(O)—C₁₋₂ haloalkyl and —C(O)—NH—C₁₋₃ alkyl, —C(O)—NH—Cu₂ haloalkyl. In a further preferred embodiment, R¹ is 3-pyridyl.

In a further preferred embodiment, R³ is phenyl or pyridyl, each of which is optionally substituted with one or more, preferably one or two, substituents selected from halogen, —C₁₋₆ alkyl, C₁₋₆ haloalkyl, —O—C₁₋₆ alkyl, and —O—C₁₋₆ haloalkyl. In a further preferred embodiment, R³ is phenyl or pyridyl, each of which is optionally substituted with one or more, preferably one or two, substituents selected from halogen, —C₁₋₃ alkyl, C₁₋₂ haloalkyl, —O—C₁₋₂ alkyl, and —O—C₁₋₃ haloalkyl. In a further preferred embodiment, R³ is phenyl or pyridyl, each of which is optionally substituted with one or more, preferably one or two, substituents selected from —F, —Cl, —C₁₋₂ alkyl, C₁ haloalkyl, —OCH₃. In a further preferred embodiment, R³ is phenyl or pyridyl, each of which is optionally substituted with one or more, preferably one or two, substituents selected from —F, —Cl, —CH₃ and —OCH₃. In a further preferred embodiment, R³ is phenyl or pyridyl, each of which is optionally substituted with one substituent selected from —F, —Cl, —CH₃ and —OCH₃. In a further preferred embodiment, R³ is phenyl or 3-pyridyl or 4-pyridyl, each of which is optionally substituted with one substituent selected from —F, —Cl, —CH₃ and —OCH₃. In a further preferred embodiment, R³ is phenyl, 3-pyridyl or 4-pyridyl, each of which is optionally substituted at the meta position of said phenyl, 3-pyridyl or 4-pyridyl with one substituent selected from —F, —Cl, —CH₃ and —OCH₃. In a further preferred embodiment, R³ is phenyl or phenyl substituted at the meta position with one substituent selected from —F, —Cl, —CH₃ and —OCH₃. In a further preferred embodiment, R³ is 3-pyridyl or 3-pyridyl substituted at the meta position (5 position) with one substituent selected from —F, —Cl, —CH₃ and —OCH₃. In a further preferred embodiment, R³ is 4-pyridyl or 4-pyridyl substituted at the meta position (5 position) with one substituent selected from —F, —Cl, —CH₃ and —OCH₃.

In a further aspect and embodiment, the present invention provides a compound of formula (I), wherein said compound of formula (I) is a compound of formula (VIc), preferably (VId), and further preferably (Vie), optionally in the form of a pharmaceutically acceptable salt, solvate, cocrystal, tautomer, racemate, enantiomer, or diastereomer or mixture thereof

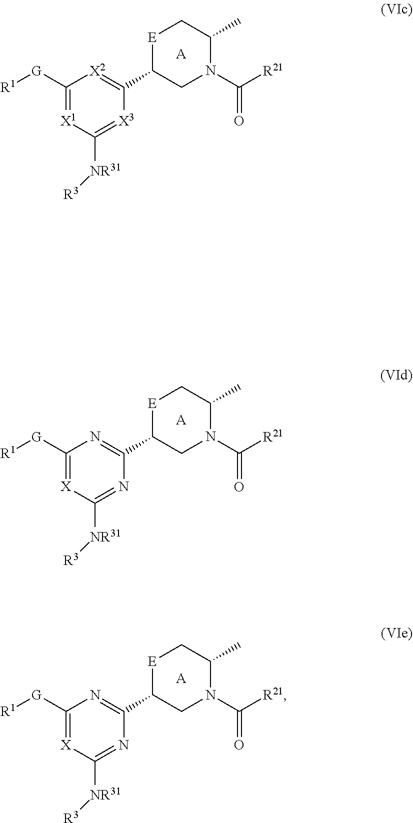

wherein
R¹ is selected from halogen and -(optionally substituted hydrocarbon group which contains from 1 to 20 carbon atoms and optionally 1 to 15 heteroatoms selected from O, N and S); R²¹ is selected from hydrogen, -(optionally substituted C₁₋₆ alkyl) which may contain one to three oxygen atoms between carbon atoms, and -(optionally substituted C₃₋₆ cycloalkyl); R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked; preferably G is a bond;
each of X¹, X² and X³ is independently selected from N, CH and CR^{x}; wherein preferably at least one of said X¹, X² and X³ is N, and wherein further preferably at least one of said X² and X³ is N; alternatively X is selected from N, CH and CR^{x}, preferably X is CH;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}— and —O—, -L¹-L²- and -L²-L¹-, wherein L¹ is selected from —CH₂—, —CHR^{x}—, —CR^{x}₂—, —NH—, NR^{x}— and —O— and L² is selected from —CH₂—, —CHR^{x}— and —CR^{x}₂—;
R^{6x} is -halogen, —OH, ═O, C₁₋₆ alkyl, C₁₋₆ haloalkyl, C₁₋₆ alkyl substituted with one or more OH, monocyclic aryl optionally substituted with one or more R^{xb}, monocyclic heteroaryl optionally substituted with one or more R^{xb}, monocyclic cycloalkyl optionally substituted with one or more R^{xb}, monocyclic heterocycloalkyl optionally substituted with one or more R^{xb}, monocyclic cycloalkenyl optionally substituted with one or more R^{xb}, monocyclic heterocycloalkenyl optionally substituted with one or more R^{xb}, wherein said R^{xb} is independently selected from -halogen, —OH, ═O, C₁₋₄ alkyl, C₁₋₂ haloalkyl, C₁₋₂ alkyl substituted with one or two OH;
wherein Ring A may further be substituted with one or more groups R^{x}, wherein any two R^{x} groups at ring A can be optionally linked and/or any R^{x} group at ring A can be optionally linked with R²; and/or wherein Ring A may be further substituted with one group R^{x} so as to form together with said methyl substitution group of Ring A a bicyclic moiety having the following partial structure:

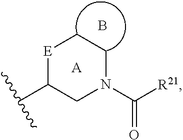

preferably

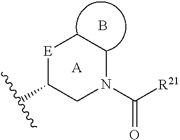

wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, oxo, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked,
with preferably the proviso that at least one, further preferably all of the compounds (a) to (bj) are excluded. In a further preferred embodiment, at least one of said X¹, X² and X³ is N. In a further preferred embodiment, both X² and X³ are nitrogen. In a further preferred embodiment, X¹ is CH.

In a further preferred embodiment, said R³¹ is selected from -hydrogen, —C₁₋₄-alkyl, and —C₁₋₂-fluoroalkyl. In a further preferred embodiment, said R³¹ is selected from -hydrogen, —C₁₋₂-alkyl, and —C₁-fluoroalkyl. In a further preferred embodiment, said R³¹ is selected from -hydrogen and methyl. In a further preferred embodiment, said R³¹ is -hydrogen.

In a preferred embodiment, said R²¹ is selected from hydrogen, C₁₋₆ alkyl, C₁₋₆ haloalkyl, C₁₋₆ alkyl optionally substituted with one or more OH, C₁₋₆ alkyl containing one to three oxygen atoms between carbon atoms, and C₃₋₆ cycloalkyl optionally substituted with one or more R²², wherein R²² is selected from halogen, preferably —Cl, —F, and —OH;

In a further preferred embodiment, said R²¹ is selected from hydrogen, C₁₋₂ alkyl, C₁₋₂ haloalkyl, C₁₋₂ alkyl optionally substituted with one or two OH, and C₃₋₄ cycloalkyl optionally substituted with one or more R²², wherein R²² is selected from —Cl, —F, and —OH.

In a further preferred embodiment, said R²¹ is selected from C₁₋₂ alkyl and cyclopropyl.

In a further preferred embodiment, said R²¹ is methyl. In a further preferred embodiment, said R²¹ is ethyl. In a further preferred embodiment, said R²¹ is cyclopropyl.

It is to be understood that Ring A may be substituted with one or more groups R^{x}, wherein any two R^{x} groups, preferably adjacent R^{x} groups, at ring A are optionally linked and/or any R^{x} group at ring A is optionally linked with R²¹; the number of groups R^{x} in Ring A is preferably 0 or 1, or preferably 0, 1, or 2. In case that Ring A may be substituted with one or more groups R^{x} and one of said R^{x} group at ring A is optionally linked with R²¹ then said one of said R^{x} group at ring A optionally linked with R²¹ is a substituent at the 2-position of Ring A.

In a further preferred embodiment, said E is selected from —CH₂—, —CHR^{x}—, —CR^{x}₂—, —NH—, —NR^{x}— and —O—. In a further preferred embodiment, said E is selected from —CH₂—, —CHR^{x}—, —CR^{x}₂—, —NH—, —NR^{x}— and —O—. More preferably, E is selected from —CH₂—, —NH— and —O—. Even more preferably, E is CH₂.

In a further preferred embodiment, said E is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—. In a further preferred embodiment, said E is —CH₂—, —CHCH₃—, —C(CH₃)₂—, —NH—, —N(CH₃)—, —O—, -L¹-L²- and -L²-L¹-, wherein L¹ is selected from —CH₂—, —CHCH₃—, —C(CH₃)₂—, —NH—, —N(CH₃)—, and —O— and L² is selected from —CH₂—, —CHCH₃—, —C(CH₃)₂—. In a further preferred embodiment, said E is —CH₂—, —CHCH₃—, —NH—, —N(CH₃)—, —O—, -L¹-L²- and -L²-L¹-, wherein L¹ is selected from —CH₂—, —CHCH₃—, —NH—, —N(CH₃)—, and —O— and L² is selected from —CH₂— and —CHCH₃—

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a preferred embodiment, each R^{x} is independently selected from -halogen, —OH, —O—C₁₋₃ alkyl optionally substituted with one or more R^{xa}, —NH—C₁₋₃ alkyl optionally substituted with one or more R^{xa}, —N(C₁₋₃ alkyl optionally substituted with one or more R^{xa})₂, ═O, C₁₋₄ alkyl optionally substituted with one or more R^{xa}, C₁₋₄ haloalkyl, —(C₁₋₂ alkylene optionally substituted with one or more R^{xa})-(optionally substituted carbocyclyl), —(C₁₋₂ alkylene optionally substituted with one or more R^{xa})-(optionally substituted heterocyclyl), —O—(C₁₋₂ alkylene optionally substituted with one or more R^{xa})-(optionally substituted carbocyclyl), —O—(C₁₋₂ alkylene optionally substituted with one or more R^{xa})-(optionally substituted heterocyclyl), -(optionally substituted carbocyclyl) and -(optionally substituted heterocyclyl), wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —(C₁₋₂ alkylene optionally substituted with one or more R^{xa})-(monocyclic carbocyclyl optionally substituted with one or more R^{xa}), —(C₁₋₂ alkylene optionally substituted with one or more R^{xa})-(monocyclic heterocyclyl optionally substituted with one or more R^{xa}), —O—(C₁₋₂ alkylene optionally substituted with one or more R^{xa})-(monocyclic carbocyclyl optionally substituted with one or more R^{xa}), —O—(C₁₋₂ alkylene optionally substituted with one or more R^{xa})-(monocyclic heterocyclyl optionally substituted with one or more R^{xa}), monocyclic carbocyclyl optionally substituted with one or more R^{xa}, monocyclic heterocyclyl optionally substituted with one or more R^{xa}, wherein said R^{xa} is independently selected from halogen, preferably —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein said R^{xa} is independently selected from —Cl, —F, and —OH.

In a further preferred embodiment, each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

In a further preferred embodiment, each R^{x} is independently selected from -halogen, —OH, —O—C₁₋₂ alkyl, —NH—C₁₋₂ alkyl, —N(C₁₋₂ alkyl)₂, ═O, C₁₋₃ alkyl, C₁₋₂ haloalkyl, —W-(monocyclic carbocyclyl optionally substituted with one R^{xa}), —W-(monocyclic heterocyclyl optionally substituted with one R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —F, and —OH.

It is to be understood that said Ring A may further be substituted with one group R^{x} so as to form together with said methyl substitution group of Ring A a bicyclic moiety having the following partial structure:

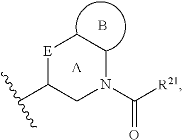

preferably

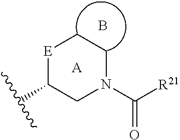

wherein, in a preferred embodiment, said Ring B is an optionally substituted cycloalkyl, optionally substituted cycloalkenyl, optionally substituted heterocycloalkyl, or optionally substituted heterocycloalkenyl, wherein said optional substituent of said cycloalkyl, cycloalkenyl, heterocycloalkyl, heterocycloalkenyl is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted cycloalkyl or an optionally substituted heterocycloalkyl, wherein said optional substituent of said cycloalkyl or said heterocycloalkyl, is independently selected from —C₁₋₄ alkyl, —C₁₋₂ haloalkyl, -halogen, -oxo, —NR*R*, —OR*; wherein each R* is independently selected from H and C₁₋₄ alkyl. In a further preferred embodiment, said Ring B is an optionally substituted monocyclic cycloalkyl or an optionally substituted monocyclic heterocycloalkyl, wherein said optional substituent of said monocyclic cycloalkyl or said monocyclic heterocycloalkyl is independently selected from —C₁₋₄ alkyl, —C₁₋₂ haloalkyl, -halogen, -oxo, —NR*R*, —OR*; wherein each R* is independently selected from H and C₁₋₄ alkyl.

Specific examples of preferred compounds of the present invention are the following:

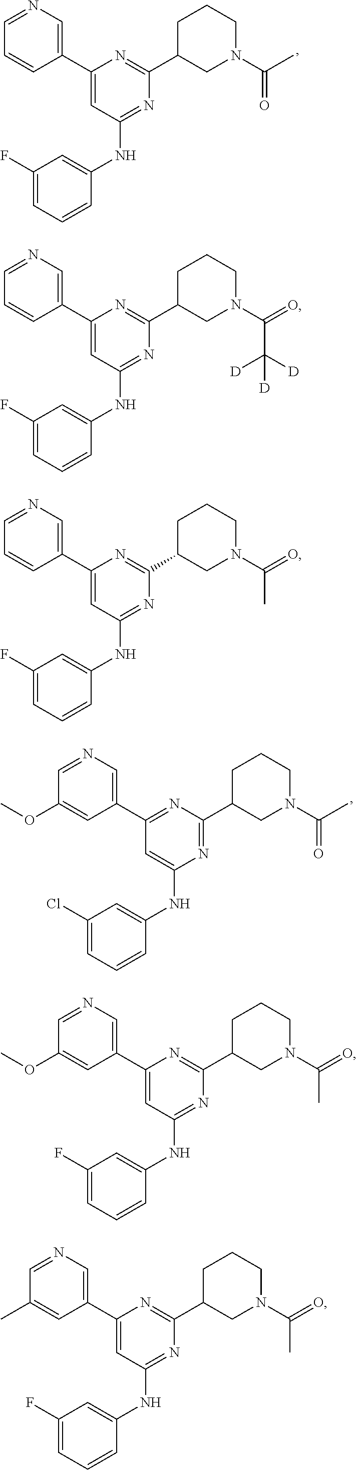

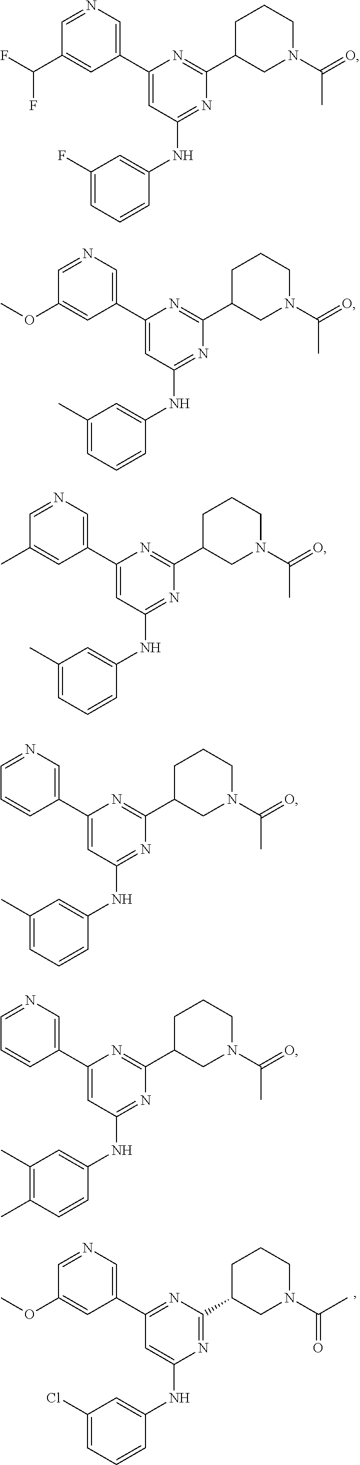

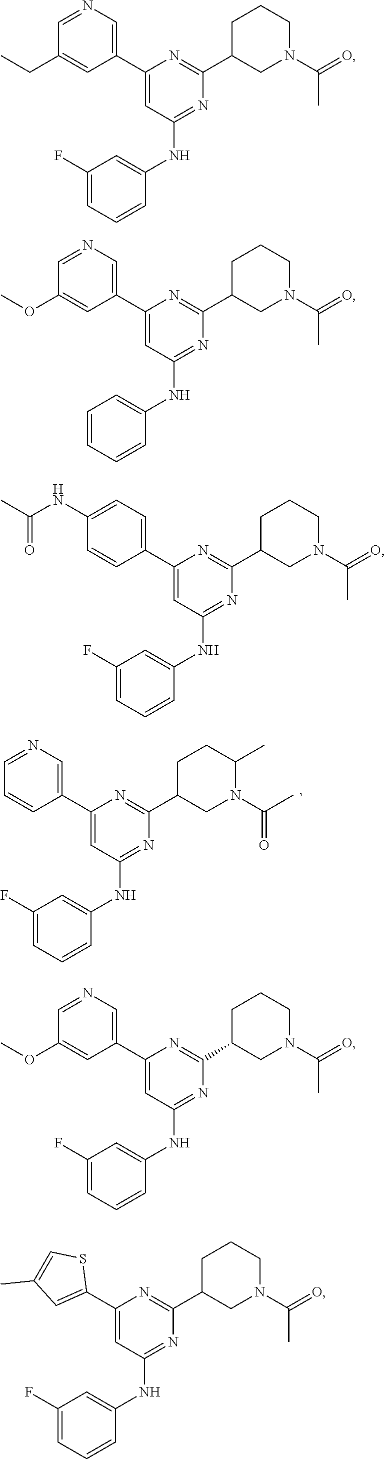

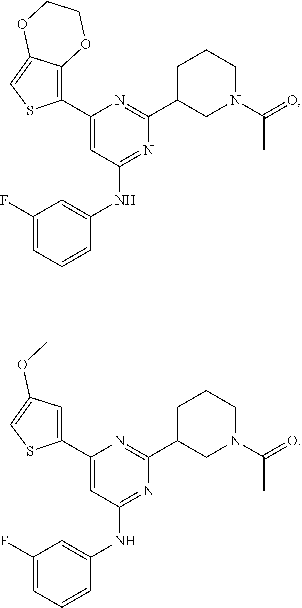 [SMILES: CC(=O)N1CCCC(c2nc(Nc3cccc(F)c3)cc(-c3scc4c3OCCO4)n2)C1 ; COc1csc(-c2cc(Nc3cccc(F)c3)nc(C3CCCN(C(C)=O)C3)n2)c1]

In one embodiment, compounds of formula (I) in which R¹-G is an unsubstituted pyridyl group are excluded.

One or more, preferably all, of the following compounds, including any pharmaceutically acceptable salt, solvate, cocrystal, tautomer, racemate, enantiomer, or diastereomer or mixture thereof, can be excluded from the compounds of formula (I) and of the invention:

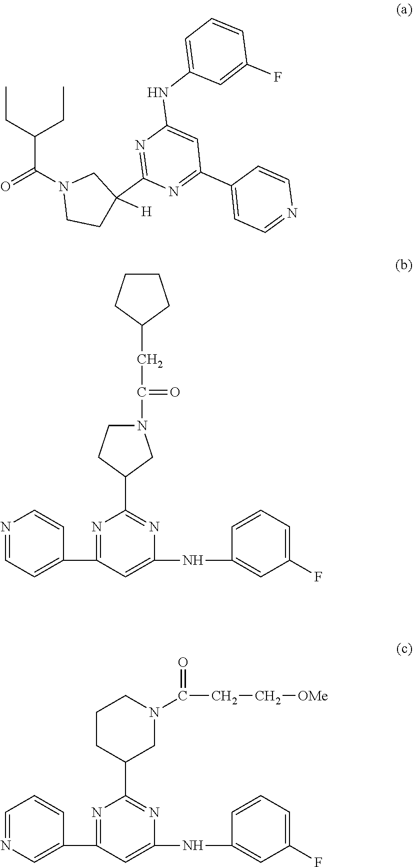 [SMILES: CCC(CC)C(=O)N1CCC(c2nc(Nc3cccc(F)c3)cc(-c3ccncc3)n2)C1 ; O=C(CC1CCCC1)N1CCC(c2nc(Nc3cccc(F)c3)cc(-c3ccncc3)n2)C1 ; COCCC(=O)N1CCCC(c2nc(Nc3cccc(F)c3)cc(-c3cccnc3)n2)C1]

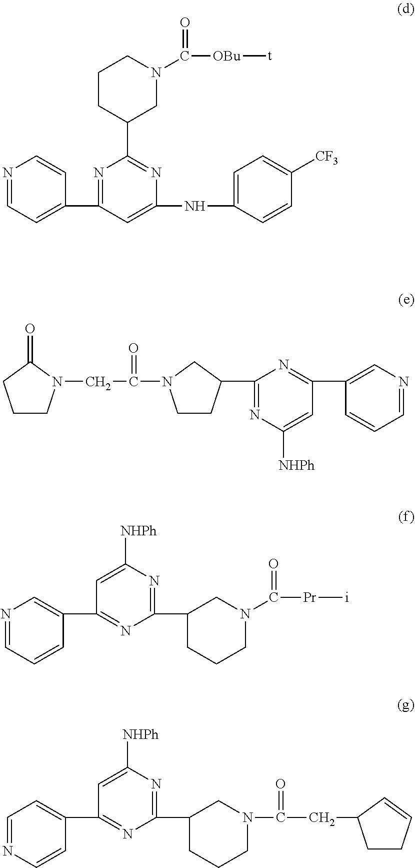

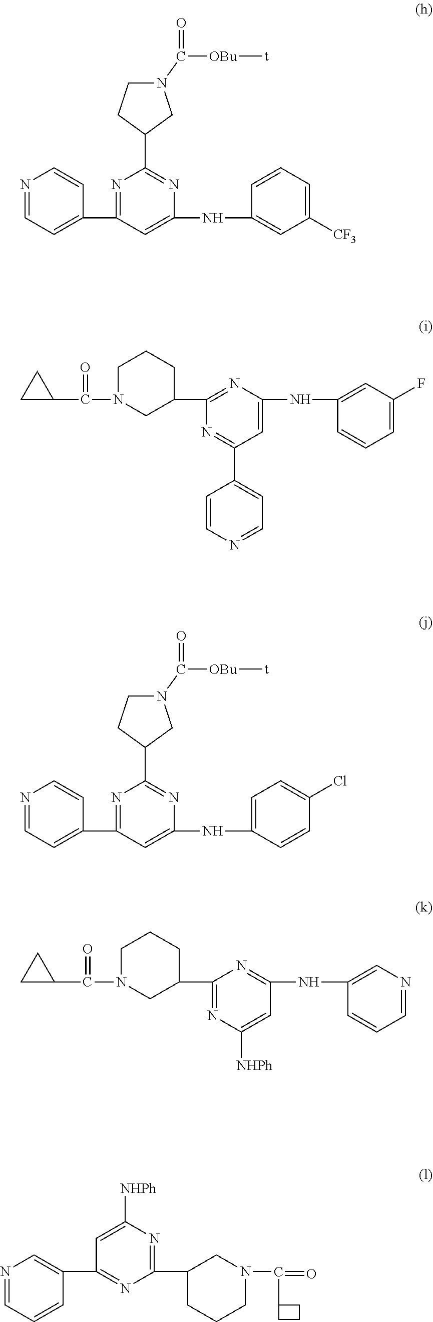

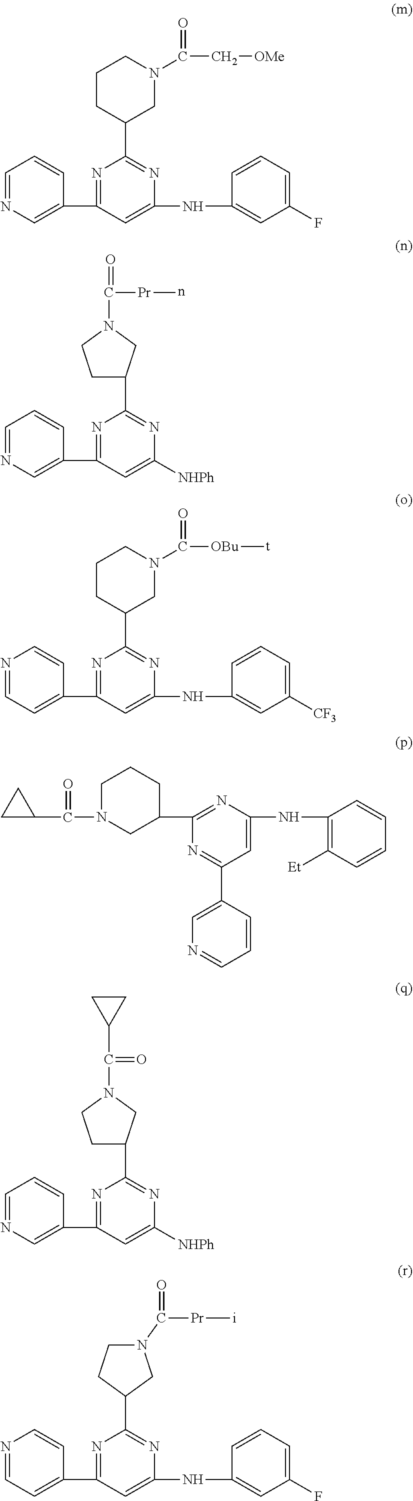

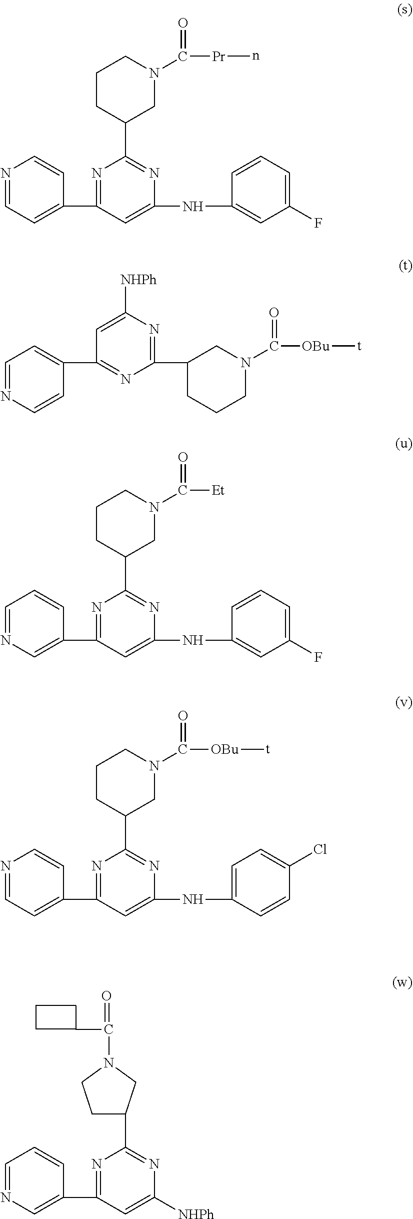

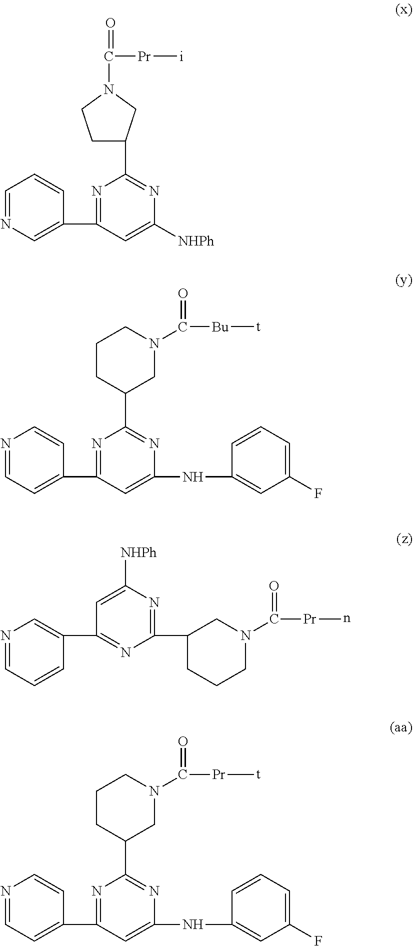

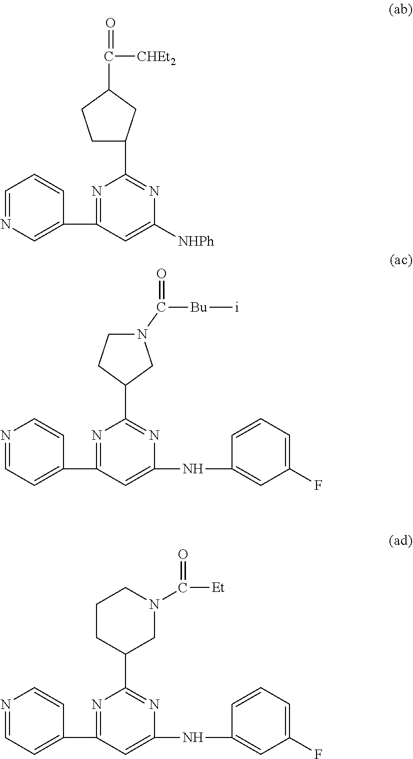

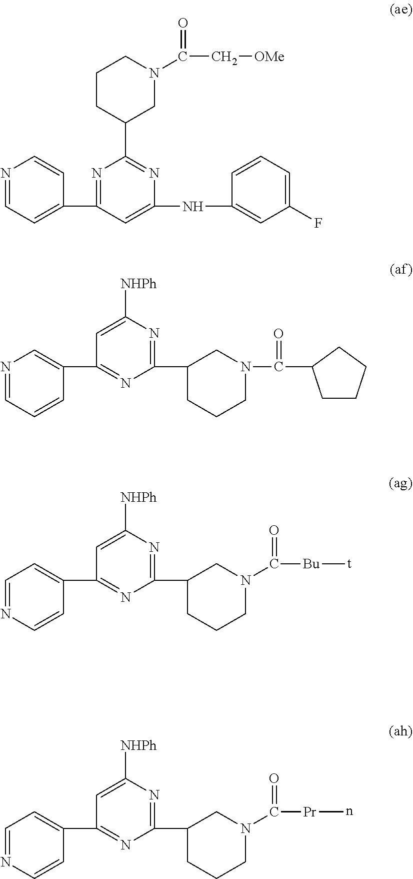

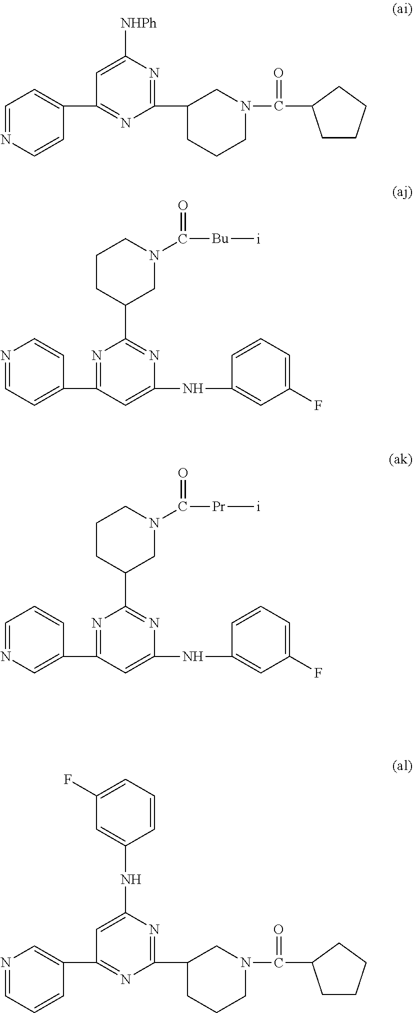

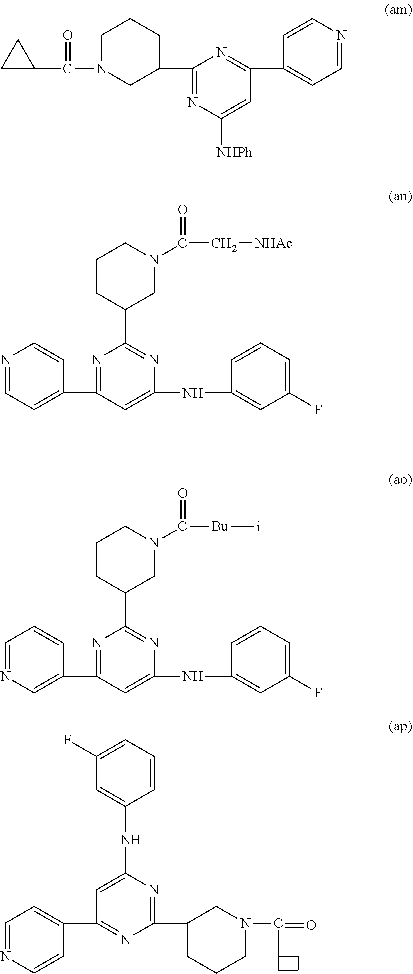

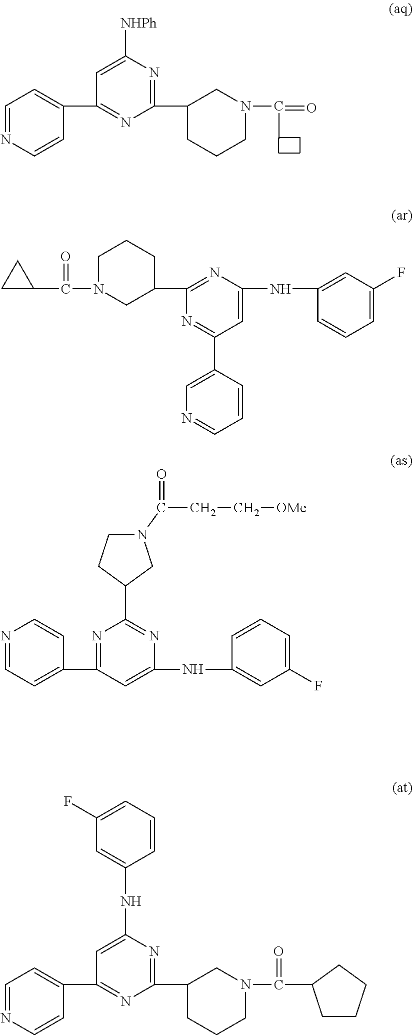

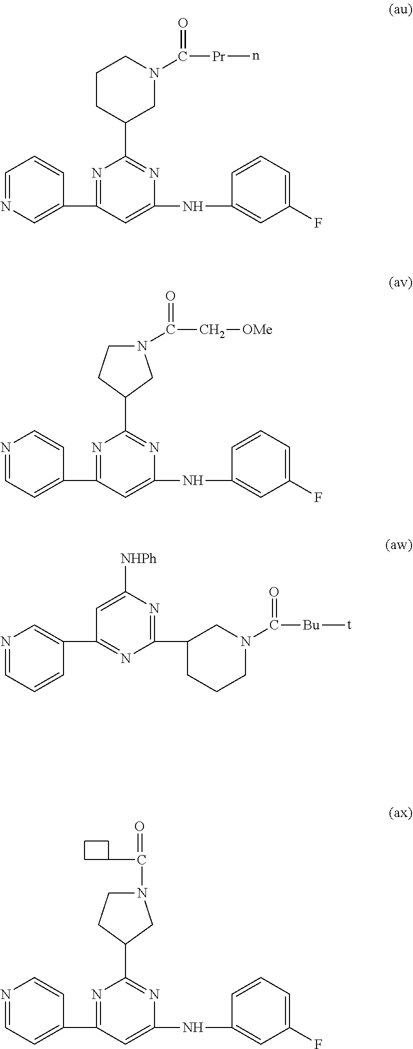

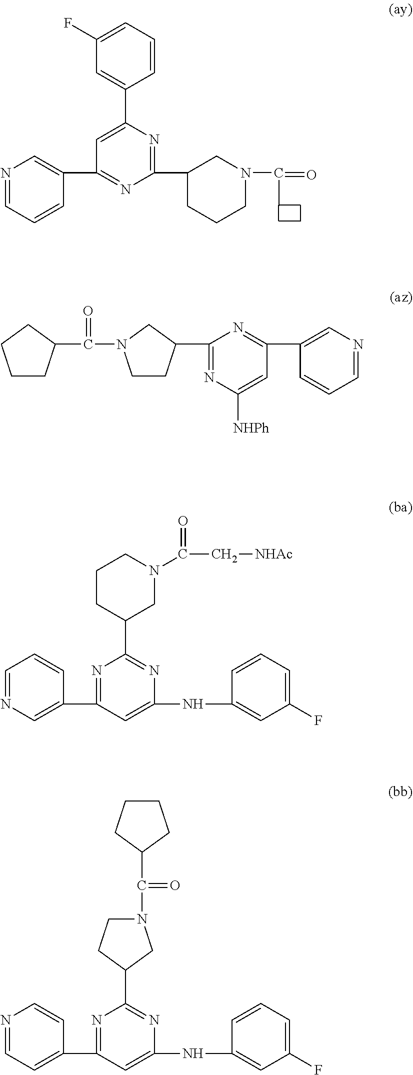

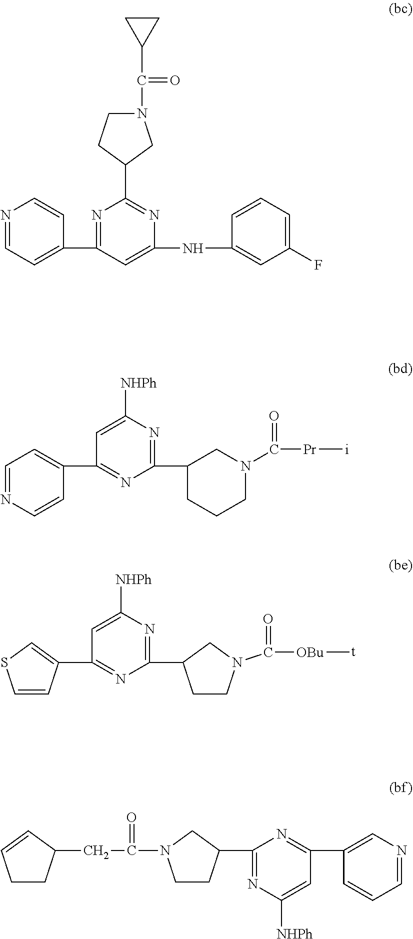

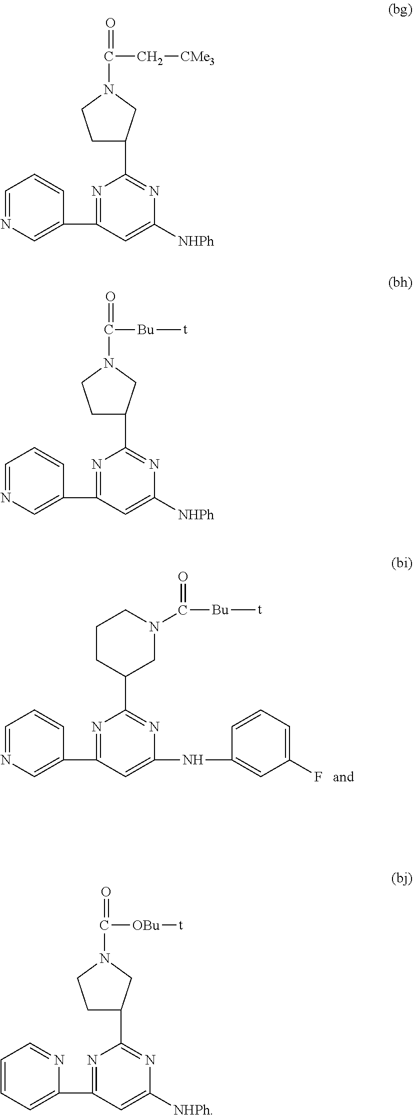

These compounds can be disclaimed individually or in total not only from the product claims but also from any other claim category.

The present inventors have surprisingly found that the compounds of the present invention bind to p300 (also called EP300 or E1A binding protein p300) and CBP (also known as CREB-binding protein or CREBBP) which are two structurally very similar transcriptional co-activating proteins. Without wishing to be limited by theory, it is believed that this binding is a main reason for the activity of the compounds of the present invention as set out herein. It is furthermore believed that the compounds of the present invention bind to the bromodomains of p300 and CBP.

It is therefore preferred that the compounds of the present invention, namely the compounds as defined in claim 1, bind to the bromodomain of p300 and/or the bromodomain of CBP with an EC50 of 10000 nM or less, preferably 2000 nM or less, more preferably 1000 nM or less, even more preferably 500 nM or less, still more preferably 200 nM or less, still more preferably 100 nM or less, still more preferably 50 nM or less, still more preferably 20 nM or less, still more preferably 10 nM or less.

The present invention furthermore relates to a pharmaceutical composition comprising a compound having the formula (I) as defined herein, optionally in the form of a pharmaceutically acceptable salt, solvate, cocrystal, tautomer, racemate, enantiomer, or diastereomer or mixture thereof, and optionally one or more pharmaceutically acceptable excipient(s) and/or carrier(s). With respect to the pharmaceutical composition the following compound is preferably disclaimed:

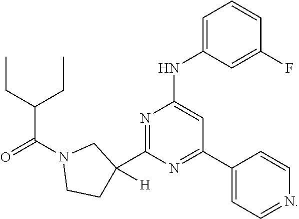 [SMILES: CCC(CC)C(=O)N1CCC(c2nc(Nc3cccc(F)c3)cc(-c3ccncc3)n2)C1]

One or more of the compounds in the above disclaimer can also be optionally disclaimed.

In addition, the present invention provides the compound having the formula (I) as defined herein, optionally in the form of a pharmaceutically acceptable salt, solvate, cocrystal, tautomer, racemate, enantiomer, or diastereomer or mixture thereof, wherein the compound is for use in the treatment, amelioration or prevention of cancer. In this medical use, one or more of the above disclaimers may or may not apply.

The present invention also relates to a method of treating, ameliorating or preventing cancer, the method comprising administering to a patient in need thereof a therapeutically effective amount of a compound having the formula (I), optionally in the form of a pharmaceutically acceptable salt, solvate, cocrystal, tautomer, racemate, enantiomer, or diastereomer or mixture thereof. In this medical use, one or more of the above disclaimers may or may not apply.

Furthermore, the present invention provides the use of the compound having the formula (I) as defined herein, optionally in the form of a pharmaceutically acceptable salt, solvate, cocrystal, tautomer, racemate, enantiomer, or diastereomer or mixture thereof, for the manufacture of a medicament for the treatment, amelioration or prevention of cancer. In this medical use, one or more of the above disclaimers may or may not apply.

The type of cancer that can be treated with the compounds and compositions of the present invention is typically selected from non-melanoma skin cancer, esophagogastric adenocarcinoma, glioblastoma, bladder cancer, bladder urothelial carcinoma, esophagogastric cancer, melanoma, non-small cell lung cancer, endometrial cancer, cervical adenocarcinoma, esophageal squamous cell carcinoma, breast cancer, head and neck squamous cell carcinoma, germ cell tumor, small cell lung cancer, ovarian cancer, soft tissue sarcoma, hepatocellular carcinoma, colorectal adenocarcinoma, cervical squamous cell carcinoma, cholangiocarcinoma, prostate cancer, upper tract urothelial carcinoma, diffuse glioma, colorectal cancer, ampullary carcinoma, adrenocortical carcinoma, head and neck cancer, renal clear cell carcinoma, hepatobiliary cancer, glioma, non-Hodgkin lymphoma, mesothelioma, salivary gland cancer, renal non-clear cell carcinoma, miscellaneous neuroepithelial tumor, pheochromocytoma, thymic tumor, multiple myeloma, renal cell carcinoma, bone cancer, pancreatic cancer, leukemia, peripheral nervous system tumors, thyroid cancer, B-lymphoblast leukemia, monoclonal B-cell lymphocytosis, lymphoma, hairy cell leukemia, acute myeloid leukemia, Wilms tumor in particular melanoma and non-small cell lung cancer, in particular melanoma and non-small cell lung cancer. The above diseases typically exhibit a mutation incidence of more than 3% of RTKs (EGFR, ERBB2, ERBB3, ERBB4, PDGFA, PDGFB, PDGFRA, PDGFRB, KIT, FGF1, FGFR1, IGF1, IGFR, VEGFA, VEGFB, KDR) and/or MAPK pathway members (KRAS, HRAS, BRAF, RAF1, MAP3K1/2/3/4/5, MAP2K1/2/3/4/5, MAPK1/3/4/6/7/8/9/12/14, DAB, RASSF1, RAB25).

In a further embodiment, the tumor may be adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia {e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, vulva, or acoustic neuroma, acute leukemia, acute lymphocytic leukemia, acute myelocytic leukemia, acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer (NSCLC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, s)movioma, sweat gland carcinoma, thyroid cancer, Waldenstrom's macroglobulinemia, testicular tumors, uterine cancer, or Wilms' tumor.

The tumour may also be a tumour wherein AR is expressed, or in cancers in which there is activation of CBP and/or p300 function. The cancers that can be treated include those which express AR or are otherwise associated with AR, those that harbour loss of function mutations in CBP or p300 and those which have activated CBP and/or p300. Cancers that may be treated include, but are not restricted to, prostate cancer, breast cancer, bladder cancer, lung cancer, lymphoma and leukaemia. The prostate cancer may be, for instance, castration-resistant prostate cancer (CRPC). The lung cancer may be, for instance, non-small cell lung cancer or small cell lung cancer.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers, or any combination thereof.

In particular, the pharmaceutical compositions may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, and vaginal.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

Alternatively, said compounds or pharmaceutical compositions can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci. (USA) 77:4030-4034 (1980); EP0052322; EP0036676; EP088046; EP0143949; EP0142641; JP 83-118008; U.S. Pat. Nos. 4,485,045; 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to the emulsification/spray drying process disclosed in WO 99/16419 or WO 01/85136. Spray drying of solution formulations of the compounds of the present invention can be carried out, e.g., as described generally in the “Spray Drying Handbook”, 5th ed., K. Masters, John Wiley & Sons, Inc., NY (1991), and in WO 97/41833 or WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration of the compounds or pharmaceutical compositions of the present invention are oral forms of administration.

Typically, a physician will determine the dosage which will be most suitable for an individual subject. The specific dose level and frequency of dosage for any particular individual subject may be varied and will depend upon a variety of factors including the age, body weight, general health, sex, diet, mode and time of administration, rate of excretion, drug combination, the severity of the particular condition, and the individual subject undergoing therapy.

A proposed, yet non-limiting dose of the compounds according to the invention for administration to a human (of approximately 70 kg body weight) may be 0.05 to 2000 mg, preferably 0.1 mg to 1000 mg, of the active ingredient per unit dose. The unit dose may be administered, e.g., 1, 2, 3 or more times per day. The unit dose may also be administered 1 to 7 times per week, e.g., with one, two or more administration(s) per day. It will be appreciated that it may be necessary to make routine variations to the dosage depending on the age and weight of the patient/subject as well as the severity of the condition to be treated. The precise dose and also the route of administration will ultimately be at the discretion of the attendant physician.

The compounds of formula (I) can be used in combination with other therapeutic agents, including in particular other anticancer agents. When a compound of the invention is used in combination with a second therapeutic agent active against the same disease, the dose of each compound may differ from that when the compound is used alone. The combination of a compound of the present invention with a second therapeutic agent may comprise the administration of the second therapeutic agent simultaneously/concomitantly or sequentially/separately with the compound of the invention.

Preferably, the second therapeutic agent to be administered in combination with a compound of this invention is an anticancer drug. The anticancer drug to be administered in combination with a compound of formula (I) according to the present invention may, e.g., be a receptor tyrosine kinase (RTK) inhibitor, a MAP kinase inhibitor, a checkpoint kinase inhibitor, and/or, in general, an agent used in immunotherapy of cancer.

For example, many cancers are known to involve BRAF, MEK, ERK and/or EGFR expression. Thus, within the present invention the second therapeutic agent to be administered in combination with a compound of this invention, may be an inhibitor of BRAF, MEK, ERK and/or EGFR. In particular not limiting embodiments:
i) said BRAFi is vemurafenib, dabrafenib, encorafenib, LGX818, PLX4720, TAK-632, MLN2480, SB590885, XL281, BMS-908662, PLX3603, RO5185426, GSK2118436 or RAF265,
ii) said MEKi is AZD6244, trametinib, selumetinib, cobimetinib, binimetinib, MEK162, RO5126766, GDC-0623, PD 0325901, CI-1040, PD-035901, hypothemycin or TAK-733,
iii) said ERKi is ulixertinib, corynoxeine, SCH772984, XMD8-92, FR 180204, GDC-0994, ERK5-IN-1, DEL-22379, BIX 02189, ERK inhibitor (CAS No. 1049738-54-6), ERK inhibitor III (CAS No. 331656-92-9), GDC-0994, honokiol, LY3214996, CC-90003, deltonin, VRT752271, TIC10, astragaloside IV, XMD8-92, VX-11e, mogrol, or VTX11e, and/or
iv) said EGFRi is cetuximab, panitumumab, zalutumumab, nimotuzumab, matuzumab, gefitinib, erlotinib, lapatinib, neratinib, vandetanib, necitumumab, osimertinib, afatinib, AP26113, EGFR inhibitor (CAS No. 879127-07-8), EGFR/ErbB-2/ErbB-4 Inhibitor (CAS No. 881001-19-0), EGFR/ErbB-2 Inhibitor (CAS No. 179248-61-4), EGFR inhibitor II (BIBX 1382, CAS No. 196612-93-8), EGFR inhibitor III (CAS No. 733009-42-2), EGFR/ErbB-2/ErbB-4 Inhibitor II (CAS No. 944341-54-2) or PKCβII/EGFR Inhibitor (CAS No. 145915-60-2).

In particular embodiments of the invention, the second therapeutic agent administered in combination with a compound of the invention may be an immunotherapy agent, more particular immuno-oncology agent, such as, e.g. an agent targeting CD52, PD-L1, CTLA4, CD20, or PD-1. Agents that may be used in combination with a compound of the present invention include, for example, alemtuzumab, atezolizumab, ipilimumab, nivolumab, ofatumumab, pembrolizumab, rituximab.

The second therapeutic agent may also be selected from: a tumor angiogenesis inhibitor (for example, a protease inhibitor, an epidermal growth factor receptor kinase inhibitor, or a vascular endothelial growth factor receptor kinase inhibitor); a cytotoxic drug (for example, an antimetabolite, such as purine and pyrimidine analogue antimetabolites); an antimitotic agent (for example, a microtubule stabilizing drug or an antimitotic alkaloid); a platinum coordination complex; an anti-tumor antibiotic; an alkylating agent (for example, a nitrogen mustard or a nitrosourea); an endocrine agent (for example, an adrenocorticosteroid, an androgen, an anti-androgen, an estrogen, an anti-estrogen, an aromatase inhibitor, a gonadotropin-releasing hormone agonist, or a somatostatin analogue); or a compound that targets an enzyme or receptor that is overexpressed and/or otherwise involved in a specific metabolic pathway that is misregulated in the tumor cell (for example, ATP and GTP phosphodiesterase inhibitors, histone deacetylase inhibitors, protein kinase inhibitors (such as serine, threonine and tyrosine kinase inhibitors (for example, Abelson protein tyrosine kinase)) and the various growth factors, their receptors and corresponding kinase inhibitors (such as epidermal growth factor receptor (EGFR) kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (for example, cyclooxygenase-1 or cyclooxygenase-2 inhibitors), topoisomerase inhibitors (for example, topoisomerase I inhibitors or topoisomerase II inhibitors), and poly ADP ribose polymerase inhibitors (PARP inhibitors).

An alkylating agent which can be used as an anticancer drug in combination with a compound of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazines (such as temozolomide).

A platinum coordination complex which can be used as an anticancer drug in combination with a compound of the present invention may be, for example, cisplatin, carboplatin, nedaplatin, oxaliplatin, satraplatin, or triplatin tetranitrate.

A cytotoxic drug which can be used as an anticancer drug in combination with a compound of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with a compound of the present invention may be, for example, a taxane (such as docetaxel, larotaxel, ortataxel, paclitaxel/taxol, or tesetaxel), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with a compound of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin).

A tyrosine kinase inhibitor which can be used as an anticancer drug in combination with a compound of the present invention may be, for example, afatinib, acalabrutinib, alectinib, apatinib, axitinib, bosutinib, cabozantinib, canertinib, crenolanib, cediranib, crizotinib, damnacanthal, dasatinib, entospletinib, entrectinib, erlotinib, foretinib, fostamatinib, gilteritinib, glesatinib, gefitinib, ibrutinib, icotinib, imatinib, linafanib, lapatinib, lestaurtinib, motesanib, mubritinib, nintedanib, nilotinib, ONT-380, pazopanib, quizartinib, regorafenib, rociletinib, radotinib, savolitinib, sitravatinib, semaxanib, sorafenib, sunitinib, savolitinib, sitravatinibg, T790M, tesevatinib, V600E, vatalanib, vemurafenib or vandetanib.

A topoisomerase-inhibitor which can be used as an anticancer drug in combination with a compound of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

A PARP inhibitor which can be used as an anticancer drug in combination with a compound of the present invention may be, for example, BMN-673, olaparib, rucaparib, veliparib, CEP 9722, MK 4827, BGB-290, or 3-aminobenzamide.

Further anticancer drugs may also be used in combination with a compound of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, or iniparib.

Also biological drugs, like antibodies, antibody fragments, antibody constructs (for example, single-chain constructs), and/or modified antibodies (like CDR-grafted antibodies, humanized antibodies, “full humanized” antibodies, etc.) directed against cancer or tumor markers/factors/cytokines involved in proliferative diseases can be employed in co-therapy approaches with the compounds of the invention. Antibodies may, for example, be immuno-oncology antibodies, such as ado-trastuzumab, alemtuzumab, atezolizumab, avelumab, bevacizumab, blinatumomab, brentuximab, capromab, cetuximab, ipilimumab, necitumumab, nivolumab, panitumumab, pembrolizumab, pertuzumab, ramucirumab, trastuzumab, or rituximab.

The combinations referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the compound of the present invention (i.e., the compound of formula (I) or a pharmaceutically acceptable salt, solvate, cocrystal, tautomer, racemate, enantiomer, or diastereomer or mixture thereof) or the second therapeutic agent may be administered first. When administration is simultaneous, the combination may be administered either in the same pharmaceutical composition or in different pharmaceutical compositions. When combined in the same formulation, it will be appreciated that the two compounds must be stable and compatible with each other and the other components of the formulation. When formulated separately, they may be provided in any convenient formulation.

The compounds of formula (I) can also be administered in combination with physical therapy, such as radiotherapy. Radiotherapy may commence before, after, or simultaneously with administration of the compounds of the invention. For example, radiotherapy may commence 1-10 minutes, 1-10 hours or 24-72 hours after administration of the compounds. Yet, these time frames are not to be construed as limiting. The subject is exposed to radiation, preferably gamma radiation, whereby the radiation may be provided in a single dose or in multiple doses that are administered over several hours, days and/or weeks. Gamma radiation may be delivered according to standard radiotherapeutic protocols using standard dosages and regimens.

The present invention thus relates to a compound of formula (I) or a pharmaceutically acceptable salt, solvate, cocrystal, tautomer, racemate, enantiomer, or diastereomer or mixture thereof, or a pharmaceutical composition comprising any of the aforementioned entities in combination with a pharmaceutically acceptable excipient, for use in the treatment or prevention of cancer, wherein the compound or the pharmaceutical composition is to be administered in combination with an anticancer drug and/or in combination with radiotherapy.

Yet, the compounds of formula (I) can also be used in monotherapy, particularly in the monotherapeutic treatment or prevention of cancer (i.e., without administering any other anticancer agents until the treatment with the compound(s) of formula (I) is terminated). Accordingly, the invention also relates to a compound of formula (I) or a pharmaceutically acceptable salt, solvate, cocrystal, tautomer, racemate, enantiomer, or diastereomer or mixture thereof, or a pharmaceutical composition comprising any of the aforementioned entities in combination with a pharmaceutically acceptable excipient, for use in the monotherapeutic treatment or prevention of cancer.

The subject or patient, such as the subject in need of treatment or prevention, may be an animal (e.g., a non-human animal), a vertebrate animal, a mammal, a rodent (e.g., a guinea pig, a hamster, a rat, a mouse), a murine (e.g., a mouse), a canine (e.g., a dog), a feline (e.g., a cat), a porcine (e.g., a pig), an equine (e.g., a horse), a primate, a simian (e.g., a monkey or ape), a monkey (e.g., a marmoset, a baboon), an ape (e.g., a gorilla, chimpanzee, orang-utan, gibbon), or a human. In the context of this invention, it is particularly envisaged that animals are to be treated which are economically, agronomically or scientifically important. Scientifically important organisms include, but are not limited to, mice, rats, and rabbits. Lower organisms such as, e.g., fruit flies like Drosophila melagonaster and nematodes like Caenorhabditis elegans may also be used in scientific approaches. Non-limiting examples of agronomically important animals are sheep, cattle and pigs, while, for example, cats and dogs may be considered as economically important animals. Preferably, the subject/patient is a mammal; more preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orang-utan, a gibbon, a sheep, cattle, or a pig); most preferably, the subject/patient is a human.

The term “treatment” of a disorder or disease as used herein (e.g., “treatment” of cancer) is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The “amelioration” of a disorder or disease may, for example, lead to a halt in the progression of the disorder or disease or a delay in the progression of the disorder or disease.

The term “prevention” of a disorder or disease as used herein (e.g., “prevention” of cancer) is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The present invention may be better understood with reference to the following examples. These examples are intended to be representative of specific embodiments of the invention, and are not intended as limiting the scope of the invention.

### EXAMPLES

Some general routes towards the desired derivatives are depicted in the schemes below.

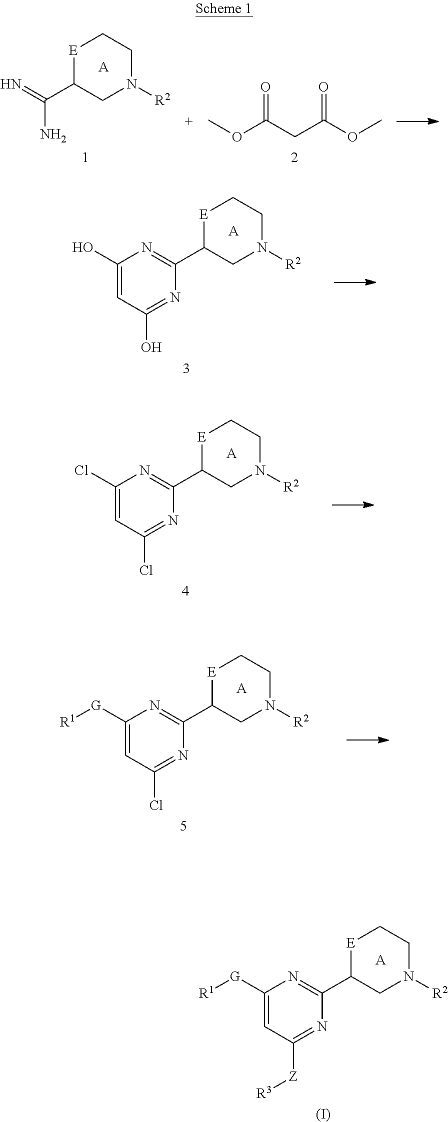

Base promoted condensation of a suitable imidate 1 with dimethylmalonate 2 afforded dihydroxy pyrimidine 3. Next, POCl₃ mediated chlorination afforded dichloropyrimidine 4. Subsequent introduction of G-R¹ for instance by Pd chemistry gave monochloro intermediate 5. Finally, introduction of Z—R₃ was effected either by Pd chemistry of thermally using for instance an aniline to afford the desired targets (1). The R² group is either present from the start or is a suitable protective group during the synthesis that can be removed and replaced by the desired moiety in a subsequent step.

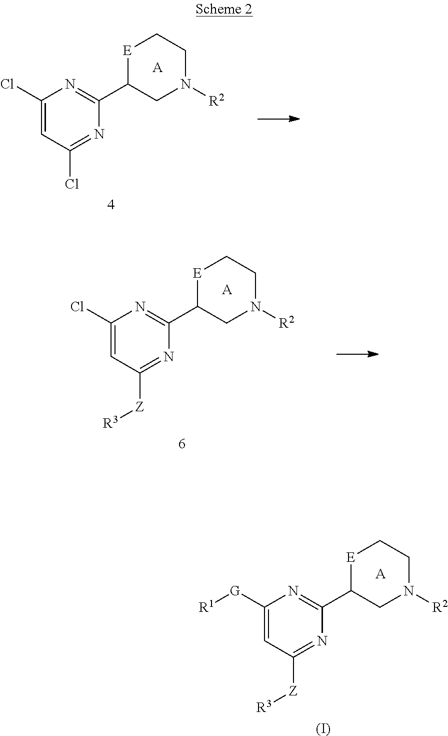

Alternatively, The Z—R³ groups were first introduced onto intermediate 4 towards intermediate 6. Then the G-R¹ group was introduced to afford the desired targets (I).

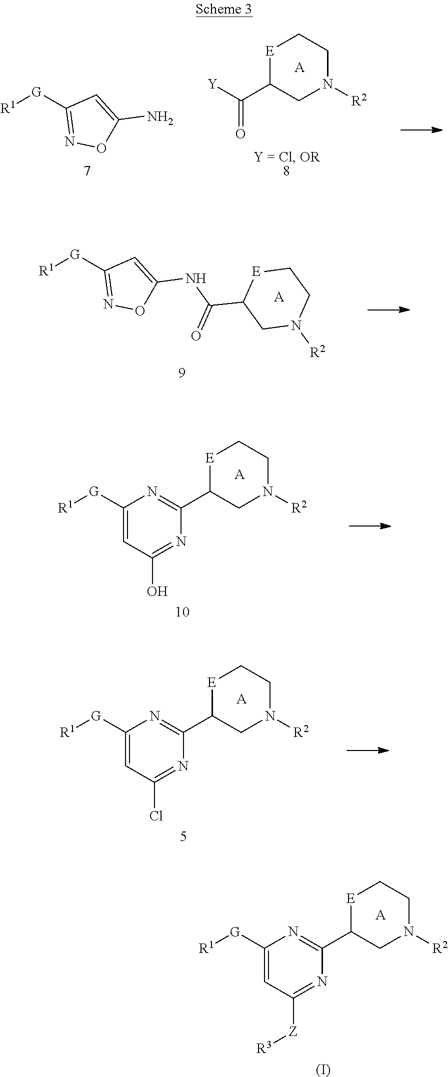

Alternatively, base promoted coupling of a suitably functionalised aniline 7 with suitably substituted 8 afforded amide 9. Subsequent rearrangement into 10 and POCl₃ mediated chlorination afforded chloropyrimidine 5 again, that could be converted into the desired targets (1) as described above.

General Experimental Methods

LCMS methods:

Method A: Apparatus: Agilent 1260 Bin. Pump: G1312B, degasser; autosampler, ColCom, DAD: Agilent G1315D, 220-320 nm, MSD: Agilent LC/MSD G6130B ESI, pos/neg 100-800, ELSD Alltech 3300 gas flow 1.5 ml/min, gas temp: 40° C.; column: Waters XSelect™ C18, 30×2.1 mm, 3.5μ, Temp: 35° C., Flow: 1 mL/min, Gradient: t₀=5% A, t_{1.6 min}=98% A, t_{3 min}=98% A, Posttime: 1.3 min, Eluent A: 0.1% formic acid in acetonitrile, Eluent B: 0.1% formic acid in water).

Method B: Apparatus: Agilent 1260 Bin. Pump: G1312B, degasser; autosampler, ColCom, DAD: Agilent G1315D, 220-320 nm, MSD: Agilent LC/MSD G6130B ESI, pos/neg 100-800, ELSD Alltech 3300 gas flow 1.5 ml/min, gas temp: 40° C.; column: Waters XSelect™ 018, 50×2.1 mm, 3.5μ, Temp: 35° C., Flow: 0.8 mL/min, Gradient: t₀=5% A, t_{3.5 min}=98% A, t_{6 min}=98% A, Posttime: 2 min; Eluent A: 0.1% formic acid in acetonitrile, Eluent B: 0.1% formic acid in water).

Method C: Apparatus: Agilent 1260 Bin. Pump: G1312B, degasser; autosampler, ColCom, DAD: Agilent G1315C, 220-320 nm, MSD: Agilent LC/MSD G6130B ESI, pos/neg 100-800; column: Waters XSelect™ CSH C18, 30×2.1 mm, 3.5μ, Temp: 25° C., Flow: 1 mL/min, Gradient: t₀=5% A, t_{1.6 min}=98% A, t_{3 min}=98% A, Posttime: 1.3 min, Eluent A: 95% acetonitrile+5% 10 mM ammoniumbicarbonate in water in acetonitrile, Eluent B: 10 mM ammoniumbicarbonate in water (pH=9.5).

Method D: Apparatus: Agilent 1260 Bin. Pump: G1312B, degasser; autosampler, ColCom, DAD: Agilent G1315C, 220-320 nm, MSD: Agilent LC/MSD G6130B ESI, pos/neg 100-800; column: Waters XSelect™ CSH C18, 50×2.1 mm, 3.5μ, Temp: 25° C., Flow: 0.8 mL/min, Gradient: t₀=5% A, t_{3.5 min}=98% A, t_{6 min}=98% A, Posttime: 2 min, Eluent A: 95% acetonitrile+5% 10 mM ammoniumbicarbonate in water in acetonitrile, Eluent B: 10 mM ammoniumbicarbonate in water (pH=9.5).

Uplc Methods:

Method A: Apparatus: Waters IClass; Bin. Pump: UPIBSM, SM: UPISMFTN with SO; UPCMA, PDA: UPPDATC, 210-320 nm, SQD: ACQ-SQD2 ESI, pos/neg 100-800; ELSD: gas pressure 40 psi, drift tube temp: 50° C.; column: Waters XSelect CSH C18, 50×2.1 mm, 2.5 μm, Temp: 25° C., Flow: 0.6 mL/min, Gradient: t₀=5% A, t_{2.0 min}=98% A, t_{2.7 min}=98% A, Posttime: 0.3 min, Eluent A: acetonitrile, Eluent B: 10 mM ammonium bicarbonate in water (pH=9.5). MS parameters: Source: ESI; Capillary: 2500 V; Cone: 15 V; Extractor: 3.0 V; RF: 2.5 V; Source Temp.: 150° C.; Desolvation Temp.: 600° C.; Cone Gas Flow: 80 L/Hr; Desolvation Gas Flow: 1000 L/Hr; Full MS scan: MS range 100-800 (positive and negative mode); scan: 0.4 sec

GCMS Methods:

Method A: Instrument: GC: Agilent 6890N G1530N and MS: MSD 5973 G2577A, EI-positive, Det.temp.: 280° C. Mass range: 50-550; Column: RXi-5MS 20 m, ID 180 μm, df 0.18 μm; Average velocity: 50 cm/s; Injection vol: 1 μl; Injector temp: 250° C.; Split ratio: 100/1; Carrier gas: He; Initial temp: 100° C.; Initial time: 1.5 min; Solvent delay: 1.0 min; Rate 75° C./min; Final temp 250° C.; Hold time 4.3 min.

Reversed Phase Chromatography

Method A: Instrument type: Reveleris™ prep MPLC; Column: Phenomenex LUNA C18 (150×25 mm, 10μ); Flow: 40 mL/min; Column temp: room temperature; Eluent A: 0.1% (v/v) formic acid in water, Eluent B: 0.1% (v/v) formic acid in acetonitrile; Gradient: t=0 min 5% B, t=1 min 5% B, t=2 min 30% B, t=17 min 70% B, t=18 min 100% B, t=23 min 100% B; Detection UV: 220/254 nm. Appropriate fractions combined and lyophilised.

Method B: Instrument type: Reveleris™ prep MPLC; Column: Waters XSelect CSH C18 (145×25 mm, 10μ); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient: t=0 min 5% B, t=1 min 5% B, t=2 min 30% B, t=17 min 70% B, t=18 min 100% B, t=23 min 100% B; Detection UV: 220/254 nm. Appropriate fractions combined and lyophilised.

Starting Materials

Standard reagents and solvents were obtained at highest commercial purity and used a such, specific reagents purchased are described below.

| Compound name | Supplier | Purity | CAS |
|---|---|---|---|
| tert-butyl 3-cyanopiperidine-1-carboxylate | Combi-Blocks | 97% | 91419-53-3 |
| Raney ®-Nickel, 50% slurry in water | Acros Organics | 50% | 7440-02-0 |
| tetrakis(triphenylphosphine)palladium(0) | Sigma-Aldrich | 98% | 14221-01-3 |
| tris(dibenzylideneacetone)dipalladium(0) | Sigma-Aldrich | 97% | 51364-51-3 |
| BrettPhos | Strem Chemicals | 98% | 1070663-78-3 |
| 1,1′-bis(diphenylphosphino)ferrocene- | Sigma-Aldrich | — | 72287-26-4 |
| palladium(II) dichloride | | | |
| Xphos | Sigma-Aldrich | 97% | 564483-18-7 |
| 3-fluorobenzylzinc chloride 0.5M in THF | Sigma-Aldrich | 0.5M | 312693-06-4 |
| bis(triphenylphosphine)palladium(II) dichloride | Fluorochem | 98% | 13965-03-2 |
| 2-(tributylstannyl)-pyrimidine | Sigma-Aldrich | 95% | 153435-63-3 |
| 10% palladium on activated carbon | ACROS | | 7440-05-3 |
| tert-butyl | Combi-Blocks | 95% | 1121057-77-9 |
| 5-(4,4,5,5-tetramethyl-1,3,2-dioxa- | | | |
| borolan-2-yl)-3,4-dihydropyridine-1(2h)- | | | |
| carboxylate | | | |
| cyanuric chloride | Sigma-Aldrich | 99% | 108-77-0 |
| methyl 3-cyanopiperidine-1-carboxylate | FCH-group | 95% | 1343196-35-9 |
| 4-benzylmorpholine-2-carbonitrile | Activate Scientific | 97% | 126645-52-1 |
| 2,6-dichloro-4-iodopyridine | Combi-Blocks | 97% | 98027-84-0 |
| 2-[(tert-butoxy)carbonyl]-2-azabicyclo[2.2.2]- | Advanced | 97% | 1936695-68-9 |
| octane-4-carboxylic acid | ChemBlocks Inc. | | |

### Synthetic Procedures for Key Intermediates

### Intermediate 1: Synthesis of 1-(3-(4,6-dichloropyrimidin-2-yl)piperidin-1-yl)propan-1-one

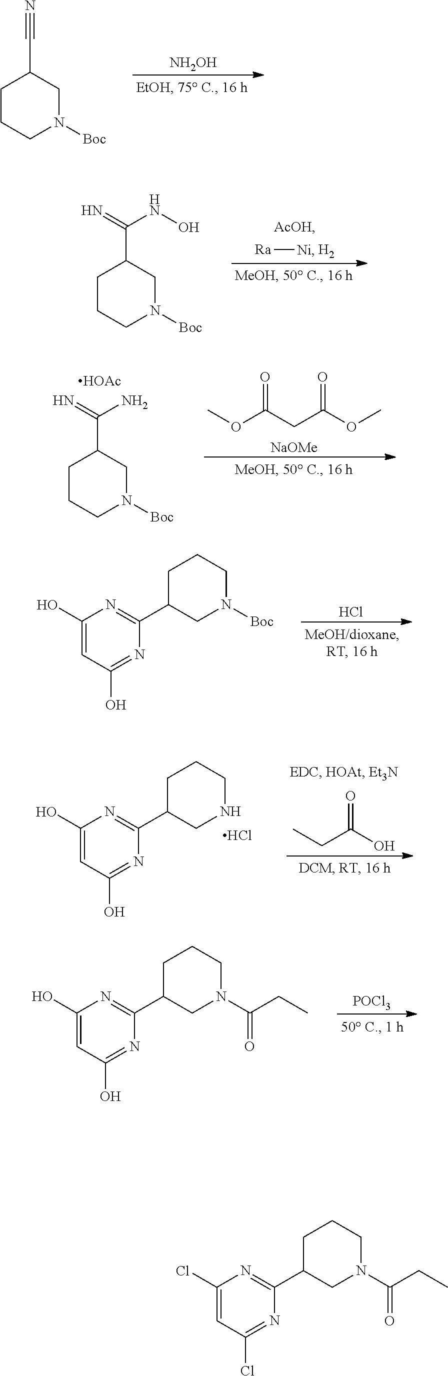

To a solution of tert-butyl 3-cyanopiperidine-1-carboxylate (50 g, 238 mmol) in ethanol (250 mL) was added hydroxylamine solution (50% in water, 43.7 mL, 713 mmol) and the mixture was stirred at 75° C. for 16 hours. The mixture was concentrated in vacuo and coevaporated with ethyl acetate twice to afford tert-butyl 3-(N-hydroxycarbamimidoyl)piperidine-1-carboxylate (58 g, 100%) as a white solid. ¹H-NMR (400 MHz, CDCl₃) δ 6.45 (br s, 1H), 4.61 (br s, 2H), 4.17 (br s, 1H), 3.99 (br s, 1H), 2.92-2.65 (m, 2H), 2.31-2.18 (m, 1H), 2.04-1.95 (m, 1H), 1.77-1.66 (m, 1H), 1.64-1.52 (m, 1H), 1.51-1.38 (m, 10H). Under argon atmosphere, tert-butyl 3-(N-hydroxycarbamimidoyl)piperidine-1-carboxylate (58 g, 238 mmol) was dissolved in methanol (500 mL) and acetic acid (41 mL, 715 mmol). Next, a 50% Raney-Nickel slurry in water (5 mL) was added, the mixture was heated to 50° C. and stirred under hydrogen atmosphere (balloon) for 16 hours. The mixture was flushed with nitrogen, filtered over Celite, washed with some MeOH and the filtrate was concentrated in vacuo to afford the crude product as a green solid. This was redissolved in a minimal amount of methanol and poured into ethyl acetate. The resulting solids were filtered off and dried to afford tert-butyl 3-carbamimidoylpiperidine-1-carboxylate acetate (28 g, 41%) as a white solid. LCMS (Method C): t_{R} 1.66 min, 100%, MS (ESI) 228.2 (M+H)⁺. Sodium (3.6 g, 157 mmol) was carefully dissolved in methanol (150 mL), followed by addition of dimethyl malonate (8.3 g, 62.6 mmol) and tert-butyl 3-carbamimidoylpiperidine-1-carboxylate acetate (15 g, 52.2 mmol). The reaction mixture was stirred at 50° C. for 16 hours, after which it was neutralised with 1M hydrochloric acid (150 mL) and partially concentrated in vacuo. The precipitate was filtered off and dried to afford tert-butyl 3-(4,6-dihydroxypyrimidin-2-yl)piperidine-1-carboxylate (18 g, 100%) as a white solid which was used without further purification in the next step. LCMS (Method A): t_{R} 1.62 min, 100%, MS (ESI) 296.1 (M+H)⁺. To a solution of tert-butyl 3-(4,6-dihydroxypyrimidin-2-yl)piperidine-1-carboxylate (18 g, 60.9 mmol) in methanol (100 mL) was added 4M hydrochloric acid in dioxane (50 mL, 200 mmol) and the mixture was stirred at room temperature for 16 hours. The mixture was concentrated in vacuo and dried to afford 2-(piperidin-3-yl)pyrimidine-4,6-diol hydrochloride (14 g, 99%) as a white solid. ¹HNMR (400 MHz, DMSO-d6): δ 10.09 (s, 2H), 9.51-9.20 (m, 2H), 5.42-5.16 (m, 1H), 3.39-3.27 (m, 1H), 3.16-3.03 (m, 3H), 2.96-2.77 (m, 1H), 2.11-1.97 (m, 1H), 1.89-1.66 (m, 2H), 1.65-1.49 (m, 1H); LCMS (Method A): t_{R} 0.10 min, 100%, MS (ESI) 196.1 (M+H)⁺. A mixture of 2-(piperidin-3-yl)pyrimidine-4,6-diol hydrochloride (5 g, 21.58 mmol), propionic acid (1.94 mL, 25.9 mmol), N-(3-dimethylaminopropyl)-N-ethylcarbodiimide hydrochloride (4.55 g, 23.74 mmol) and 1-hydroxy-7-azabenzotriazole (0.15 g, 1.08 mmol) in dichloromethane (200 mL) was stirred at room temperature for 16 hours. The mixture was concentrated in vacuo to approximately 30% of its original volume and the precipitated solids were filtered off. The filtrate was concentrated in vacuo to afford crude 1-(3-(4,6-dihydroxypyrimidin-2-yl)piperidin-1-yl)propan-1-one as a sticky yellow oil that was used as such in the next step. LCMS (Method A): t_{R} 0.88 min, 87%, MS (ESI) 250.1 (M−H)⁻. The crude 1-(3-(4,6-dihydroxypyrimidin-2-yl)piperidin-1-yl)propan-1-one from the previous step was dissolved in phosphorus oxychloride (5 mL, 53.6 mmol) and stirred at 50° C. for 1 hour. The mixture was concentrated in vacuo, poured slowly onto ice water and extracted with ethyl acetate (˜100 mL) four times. The combined organic layers were dried with sodium sulfate and concentrated in vacuo to afford a light yellow oil that was purified with silica flash column chromatography (20% to 70% ethyl acetate in n-heptane) to afford 1-(3-(4,6-dichloropyrimidin-2-yl)piperidin-1-yl)propan-1-one (3.6 g, 58%) as a colourless sticky oil. ¹H-NMR (400 MHz, chloroform-d) mixture of rotamers δ 7.29 (s, 1H), 4.92-4.79 (m, 0.5H), 4.47-4.34 (m, 0.5H), 4.06-3.95 (m, 0.5H), 3.91-3.82 (m, 0.5H), 3.58-3.45 (m, 0.5H), 3.16-2.76 (m, 2.5H), 2.51-2.31 (m, 2H), 2.27-2.13 (m, 1H), 1.99-1.82 (m, 1H), 1.82-1.70 (m, 1H), 1.57-1.50 (m, 1H), 1.21-1.08 (m, 3H); LCMS (Method B): t_{R} 3.21 min, 100%, MS (ESI) 288.0 (M+H)⁺.

### Intermediate 2: Synthesis of 1-(3-(4,6-dichloropyrimidin-2-yl)piperidin-1-yl)ethan-1-one

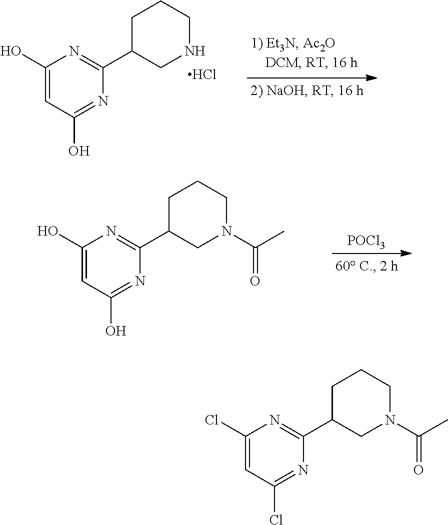

To 2-(piperidin-3-yl)pyrimidine-4,6-diol hydrochloride (4 g, 17.3 mmol) and triethylamine (4.81 mL, 34.5 mmol) in dichloromethane (250 ml), acetic anhydride (8.15 mL, 86 mmol) was added and the reaction mixture was stirred at room temperature for 16 hours. The mixture was concentrated in vacuo after which 100 mL 2N NaOH was added and the mixture was stirred at room temperature for 16 hours. The reaction mixture was neutralised with 100 mL 2N HCl and concentrated in vacuo. The organic solids were dissolved in a minimal amount of methanol, filtered and purified by reverse phase chromatography (method A) to afford 1-(3-(4,6-dihydroxypyrimidin-2-yl)piperidin-1-yl)ethan-1-one (1.7 g, 41%) as a white fluffy solid. LCMS (Method A): t_{R} 1.27 min, 100%, MS (ESI) 238.1 (M+H)⁺. A solution of 1-(3-(4,6-dihydroxypyrimidin-2-yl)piperidin-1-yl)ethan-1-one (500 mg, 2.11 mmol) in phosphorus oxychloride (10 mL, 107.5 mmol) was heated to 60° C. and stirred for 2 hours. The reaction mixture was concentrated affording a yellow oil which was carefully quenched with an ice water/saturated aqueous sodium bicarbonate mixture. The mixture was concentrated and the resulting yellow oil was partitioned between a saturated sodium bicarbonate solution and ethyl acetate. The layers were separated and the aqueous layer was extracted with ethyl acetate twice. The combined organic layers were washed with water, dried with sodium sulfate and concentrated to afford 1-(3-(4,6-dichloropyrimidin-2-yl)piperidin-1-yl)ethan-1-one (490 mg, 85%) as a sticky yellow oil. ¹H-NMR (400 MHz, DMSO-d6) mixture of rotamers δ 7.93 (d, J=7.3 Hz, 1H), 4.68-4.59 (m, 0.5H), 4.13-4.03 (m, 0.5H), 4.00-3.91 (m, 0.5H), 3.82 (d, J=13.6 Hz, 0.5H), 3.53-3.43 (m, 0.5H), 3.11-2.97 (m, 1H), 2.90-2.77 (m, 1H), 2.77-2.64 (m, 0.5H), 2.18-2.05 (m, 1H), 2.03 (d, J=4.9 Hz, 3H), 1.88-1.33 (m, 3H); LCMS (Method A): t_{R} 1.87 min, 99%, MS (ESI) 274.0 (M+H)⁺.

### Intermediate 3: Synthesis of methyl 3-(4,6-dichloropyrimidin-2-yl)piperidine-1-carboxylate

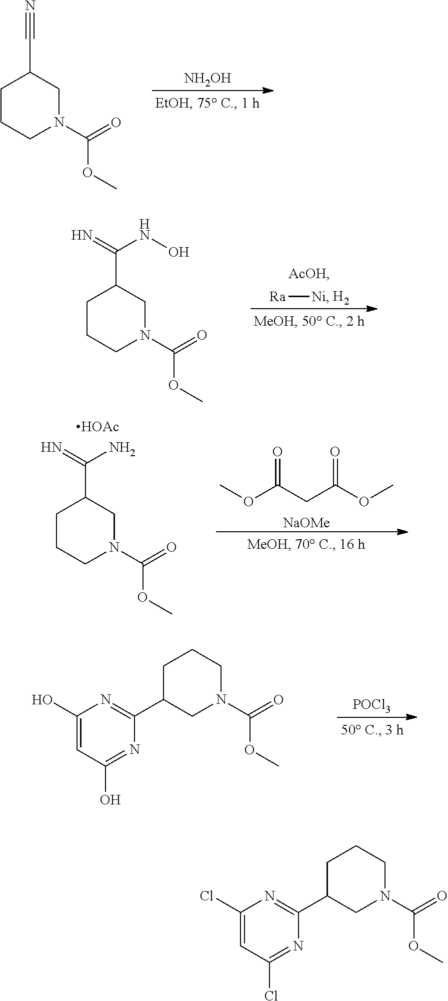

To a solution of methyl 3-cyanopiperidine-1-carboxylate (5 g, 29.7 mmol) in ethanol (50 mL) was added hydroxylamine solution (50% in water, 5.47 mL, 89 mmol) and the mixture was stirred at 75° C. for 1 hour. The mixture was concentrated in vacuo and coevaporated with ethyl acetate three times to afford methyl 3-(N-hydroxycarbamimidoyl)piperidine-1-carboxylate (5.9 g, 100%) as a colourless sticky oil, which was used without further purification in the next step. LCMS (Method A): t_{R} 0.13 min, 96%, MS (ESI) 202.1 (M+H)⁺. Under argon atmosphere, methyl 3-(N-hydroxycarbamimidoyl)piperidine-1-carboxylate (5 g, 24.8 mmol) was dissolved in a mixture of methanol (100 mL) and acetic acid (4.27 mL, 74.5 mmol). Next, a 50% Raney-Nickel slurry in water (1 mL) was added, the mixture was heated to 50° C. and stirred under hydrogen atmosphere (balloon) for 2 hours. The mixture was flushed with nitrogen, filtered over Celite, washed with some MeOH and the filtrate was concentrated in vacuo to afford a green oil. The oil was coevaporated with ethyl acetate twice to afford methyl 3-carbamimidoylpiperidine-1-carboxylate acetate (6.0 g, 100%) as a greenish solid, which was used without further purification in the next step. LCMS (Method A): t_{R} 0.28 min, 100%, MS (ESI) 186.1 (M+H)⁺. A solution of methyl 3-carbamimidoylpiperidine-1-carboxylate acetate (5 g, 20.4 mmol) and dimethyl malonate (2.96 g, 22.4 mmol) in 1M sodium methoxide in methanol (61.2 mL, 61.2 mmol) was stirred at 70° C. for 16 hours. The mixture was acidified using 1 M hydrochloric acid to approximately pH 7, concentrated in vacuo and coevaporated with ethyl acetate three times to afford methyl 3-(4,6-dihydroxypyrimidin-2-yl)piperidine-1-carboxylate (5 g, 100%) as a thick oil, which was used without further purification in the next step. LCMS (Method A): t_{R} 1.18 min, 88%, MS (ESI) 254.1 (M+H)⁺. A solution of methyl 3-(4,6-dihydroxypyrimidin-2-yl)piperidine-1-carboxylate (5 g, 19.7 mmol) was dissolved in phosphorus oxychloride (25 mL, 268 mmol) and stirred at 50° C. for 3 hours. The mixture was concentrated, the residue was partitioned between ice water and ethyl acetate and the layers were separated. The aqueous layer was extracted with ethyl acetate twice and the combined organic layers were concentrated in vacuo to afford a yellow oil. This was purified by silica flash column chromatography (20% to 50% ethyl acetate in n-heptane) to afford methyl 3-(4,6-dichloropyrimidin-2-yl)piperidine-1-carboxylate (2.2 g, 38%) as a colourless oil. ¹H-NMR (400 MHz, chloroform-d) δ 7.27 (s, 1H), 4.53-4.26 (m, 1H), 4.25-4.05 (m, 1H), 3.71 (s, 3H), 3.22-3.07 (m, 1H), 3.07-2.94 (m, 1H), 2.91-2.77 (m, 1H), 2.25-2.15 (m, 1H), 1.84-1.68 (m, 2H), 1.66-1.52 (m, 1H); LCMS (Method A): t_{R} 1.99 min, 100%, MS (ESI) 290.0 (M+H)⁺.

### Intermediate 4: Synthesis of methyl 3-(4,6-dichloropyrimidin-2-yl)piperidine-1-carboxylate

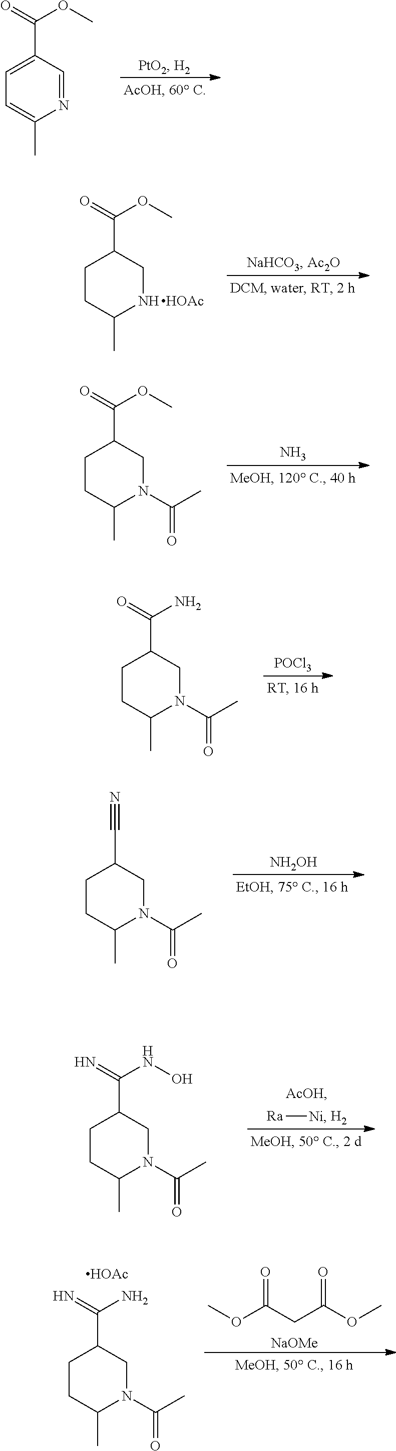

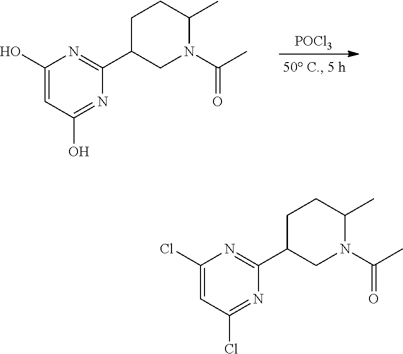 [SMILES: CC(=O)N1CC(c2nc(O)cc(O)n2)CCC1C ; "POCl3" ; CC(=O)N1CC(c2nc(Cl)cc(Cl)n2)CCC1C]

To a solution of methyl 6-methylnicotinate (100 g, 662 mmol) in acetic acid (250 mL) in a 1 L steel autoclave, platinum(IV) oxide (0.5 g, 2.202 mmol) was added after which the reaction mixture was stirred under 10 bar hydrogen atmosphere at 60° C. Rapid hydrogen consumption was observed and the autoclave was refilled several times until hydrogen consumption stopped and the reduction was complete. The mixture was cooled to room temperature and filtrated over Celite. The filtrate was concentrated to afford methyl 6-methylpiperidine-3-carboxylate acetate as a mixture of diastereoisomers (143.8 g, 100%) that was used as such in the next step. GCMS (Method A): t_{R} 2.40 (80%) and 2.48 min (20%), 100%, MS (EI) 157.1 (M)+, 142.1 (M-Me)+. To a solution of methyl 6-methylpiperidine-3-carboxylate acetate (53 g, 244 mmol) in a mixture of water (500 mL) and dichloromethane (500 mL), sodium bicarbonate (82 g, 976 mmol) was added carefully (effervescence!!) after which acetic anhydride (29.9 g, 293 mmol) was added slowly. The reaction mixture was stirred at room temperature for 2 hours. The organic layer was separated, dried on sodium sulfate, filtered and concentrated in vacuo to afford methyl 1-acetyl-6-methylpiperidine-3-carboxylate (49 g, 100%) as a yellow oil. ¹H NMR (400 MHz, Chloroform-d) mixture of diastereoisomers and rotamers δ 5.01-4.86 (m, 0.5H), 4.82-4.70 (m, 0.5H), 4.19-4.04 (m, 0.5H), 3.86-3.76 (m, 0.5H), 3.75-3.65 (m, 3H), 3.37-3.14 (m, 0.5H), 2.81-2.67 (m, 0.5H), 2.49-2.32 (m, 1H), 2.19-2.03 (m, 3H), 2.02-1.89 (m, 1H), 1.89-1.53 (m, 3H), 1.30-1.07 (m, 3H). A solution of methyl 1-acetyl-6-methylpiperidine-3-carboxylate (49 g, 246 mmol) in ammonia in methanol (7N, 500 mL, 3.5 mol) was stirred in a pressure vessel at 120° C. for 40 hours. The mixture was cooled to room temperature and concentrated to afford a light yellow solid. This was dissolved in dichloromethane and filtered over a plug of silica. The filtrate was concentrated to afford 1-acetyl-6-methylpiperidine-3-carboxamide as an off white solid that was used as such in the next step. ¹H NMR (400 MHz, DMSO-d6) δ 12.32-11.66 (m, 1H), 11.53-10.91 (m, 1H), 4.44-4.21 (m, 1H), 4.06-3.81 (m, 1H), 3.60 (s, 3H), 3.14-2.92 (m, 1H), 2.60-2.52 (m, 1H), 1.92-1.74 (m, 2H), 1.63-1.48 (m, 2H), 1.12 (d, J=6.9 Hz, 3H). A solution of 1-acetyl-6-methylpiperidine-3-carboxamide (266 mmol) from the previous step in phosphorus oxychloride (500 mL, 5.37 mol) was stirred at room temperature for 16 hours. The reaction mixture was evaporated in vacuo affording a thick oil. This was co-evaporated twice with toluene and carefully partitioned between cold saturated sodium carbonate (effervescence!) and ethyl acetate. The organic layer was separated from the basic water layer, dried on sodium sulfate, filtered and concentrated in vacuo to afford the product as a thick oil that solidified upon standing. The crude was dissolved in dichloromethane and filtered over a plug of silica (eluted with 10% methanol in dichloromethane). This afforded 1-acetyl-6-methylpiperidine-3-carbonitrile (28 g, 63%) as an oil that solidified upon standing. ¹H NMR (400 MHz, DMSO-d6) mixture of diastereoisomers and rotamers δ 4.82-4.58 (m, 0.5H), 4.57-4.44 (m, 0.5H), 4.25-3.79 (m, 1H), 3.63 (s, 0.2H), 3.31-3.20 (m, 0.6H), 3.18-3.13 (m, 0.4H), 2.95-2.57 (m, 1.2H), 2.09-1.41 (m, 7H), 1.27-0.98 (m, 3H); GCMS (Method A): t_{R} 3.78 (63%) and 3.89 min (378%), 100%, MS (EI) 166.1 (M)+. To a solution of 1-acetyl-6-methylpiperidine-3-carbonitrile (23 g, 138 mmol) in ethanol (300 ml), hydroxylamine solution (50% in water, 25.4 mL, 415 mmol) was added after which the reaction mixture was stirred at reflux for 16 hours. The reaction mixture was concentrated and co-evaporated with ethyl acetate three times to afford evaporated to dryness and stripped 3× with EtOAc to afford 1-acetyl-N-hydroxy-6-methylpiperidine-3-carboximidamide as a sticky solid. LCMS (Method A): t_{R} 0.13 min, 100%, MS (ESI) 200.2 (M+H)⁺. Assuming quantitative yield, the product was used as such in the next step. To a solution of 1-acetyl-N-hydroxy-6-methylpiperidine-3-carboximidamide (138 mmol) from the previous step in ethanol (500 mL), acetic acid (23.79 mL, 416 mmol) and 50% Raney®-Nickel slurry in water (5 mL) were added after which the reaction mixture was stirred under hydrogen atmosphere for 2 days at 50° C. The mixture was filtered over Celite, washed with some ethanol and concentrated to afford 70 g of a thick oil. This was co-evaporated twice with ethyl acetate and extensively dried in vacuo to afford 1-acetyl-6-methylpiperidine-3-carboximidamide acetate (33 g, 98%) as a greenish yellow oil that was used as such in the next step. LCMS (Method A): t_{R} 0.14 min, 90%, MS (ESI) 184.1 (M+H)⁺. To a solution of sodium (18.14 g, 789 mmol) in dry methanol under nitrogen atmosphere (60 mL) 1-acetyl-6-methylpiperidine-3-carboximidamide acetate (32 g, 132 mmol) and dimethyl malonate (26.1 g, 197 mmol) were added, after which the reaction mixture was stirred at 50° C. for 16 hours. The reaction mixture was concentrated, taken up in water (300 mL), acidified to pH4 using 6N HCl and left to crystallise overnight. The formed precipitate was filtered off to afford 1-(5-(4,6-dihydroxypyrimidin-2-yl)-2-methylpiperidin-1-yl)ethan-1-one as a yellow solid (10.4 g, 31%) that was used as such in the next step. A suspension of 1-(5-(4,6-dihydroxypyrimidin-2-yl)-2-methylpiperidin-1-yl)ethan-1-one (10.4 g, 41.4 mmol) in phosphorus oxychloride (200 mL, 2146 mmol) was stirred at 50° C. The solids slowly dissolved after approximately 3 hours. After 5 hours, the reaction mixture was concentrated in vacuo and co-evaporated with toluene twice. The remaining oil was carefully quenched with ice and neutralised with saturated aqueous sodium bicarbonate and extracted with ethyl acetate (2×100 mL). The combined organic layers were dried with sodium sulfate and concentrated in vacuo to afford 1-(5-(4,6-dichloropyrimidin-2-yl)-2-methylpiperidin-1-yl)ethan-1-one (6.8 g, 57%) as a yellow oil that solidified upon standing. ¹H-NMR (400 MHz, chloroform-d) ˜9/1 mixture of cis/trans isomers, mixture of rotamers δ 7.31 (s, 0.4H), 7.26 (s, 0.5H), 5.07-4.96 (m, 0.5H), 4.87-4.79 (m, 0.4H), 4.23-4.08 (m, 0.4H), 3.94-3.85 (m, 0.5H), 3.53-3.44 (m, 0.5H), 3.04-2.85 (m, 1.3H), 2.15 (s, 1.1H), 2.13 (s, 1.5H), 2.09-1.63 (m, 4H), 1.32 (d, J=6.9 Hz, 1.2H), 1.21 (d, J=7.0 Hz, 1.6H); LCMS (Method A): t_{R} 1.88 min, 100%, MS (ESI) 288.1 (M+H)⁺.

### Synthetic Procedures for Final Products

### Example 1: Synthesis of 1-(3-(4-((3-fluorophenyl)amino)-6-(pyridin-3-yl)pyrimidin-2-yl)piperidin-1-yl)propan-1-one (00002)

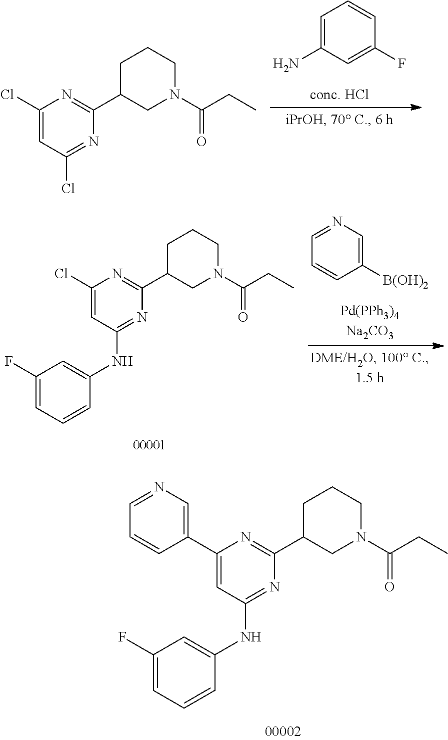

To a solution of 1-(3-(4,6-dichloropyrimidin-2-yl)piperidin-1-yl)propan-1-one (600 mg, 2.08 mmol) and 3-fluoroaniline (231 mg, 2.08 mmol) in 2-propanol (10 mL) concentrated hydrochloric acid (0.61 mL, 7.29 mmol) was added and the mixture was stirred at 70° C. for 6 hours. The mixture was concentrated in vacuo, purified by reversed phase chromatography (method A) to afford 1-(3-(4-chloro-6-((3-fluorophenyl)amino)pyrimidin-2-yl)piperidin-1-yl)propan-1-one (600 mg, 79%) as a white fluffy solid. ¹H-NMR (400 MHz, DMSO-d6) mixture of rotamers δ 10.07 (s, 1H), 7.76 (t, J=14.1 Hz, 1H), 7.46-7.23 (m, 2H), 6.89 (t, 1H), 6.71 (d, J=3.6 Hz, 1H), 4.71 (d, J=9.8 Hz, 0.5H), 4.14 (dd, J=53.2, 13.3 Hz, 1H), 3.88 (d, J=13.5 Hz, 0.5H), 3.00 (t, J=12.9 Hz, 0.5H), 2.92-2.59 (m, 2H), 2.40-2.28 (m, 2H), 2.21-2.05 (m, 1H), 1.88-1.60 (m, 2H), 1.60-1.34 (m, 1H), 1.07-0.91 (m, 3H); LCMS (Method A): t_{R} 2.02 min, 100%, MS (ESI) 363.1 (M+H)⁺. A microwave vial was charged with 1-(3-(4-chloro-6-((3-fluorophenyl)amino)pyrimidin-2-yl)piperidin-1-yl)propan-1-one (150 mg, 0.41 mmol) and pyridine-3-boronic acid (102 mg, 0.83 mmol). Next, a solution of sodium carbonate (88 mg, 0.83 mmol) in water (1 mL) and 1,2-dimethoxyethane (4 mL) was added resulting in a white suspension. Argon was bubbled through for 5 minutes, tetrakis(triphenylphosphine)palladium(0) (23.9 mg, 0.02 mmol) was added and the vial was heated in a pre-heated oil bath at 100° C. for 1.5 hours. The mixture was poured into water and extracted with ethyl acetate twice. The combined organic layers were washed with brine, dried with sodium sulfate and concentrated in vacuo to afford a yellow solid. This was purified by reversed phase chromatography (method B) to afford 1-(3-(4-((3-fluorophenyl)amino)-6-(pyridin-3-yl)pyrimidin-2-yl)piperidin-1-yl)propan-1-one (159 mg, 95%) as a white solid. ¹H-NMR (400 MHz, DMSO-d6) mixture of rotamers δ 10.02 (d, J=7.6 Hz, 1H), 9.22 (s, 1H), 8.73 (s, 1H), 8.43 (d, J=8.0 Hz, 1H), 7.89 (dd, J=20.4, 12.1 Hz, 1H), 7.61 (dd, J=8.0, 4.8 Hz, 1H), 7.49-7.33 (m, 2H), 7.19 (d, J=2.3 Hz, 1H), 6.85 (t, J=8.4 Hz, 1H), 4.77 (d, J=12.7 Hz, 0.5H), 4.23 (dd, J=27.3, 12.7 Hz, 1H), 3.90 (d, J=13.7 Hz, 0.5H), 3.06 (t, J=12.7 Hz, 0.5H), 2.90 (dd, J=21.9, 10.8 Hz, 1H), 2.84-2.71 (m, 1H), 2.44-2.31 (m, 2H), 2.23 (s, 1H), 1.99-1.70 (m, 2H), 1.64-1.36 (m, 1H), 1.07-0.94 (m, 3H); LCMS (Method B): t_{R} 3.08 min, 100%, MS (ESI) 406.2 (M+H)⁺.

The following compounds were prepared using procedures analogous to Example 1.

| Compound # | Structure and compound name | Analytical data |
|---|---|---|
| 00003 | 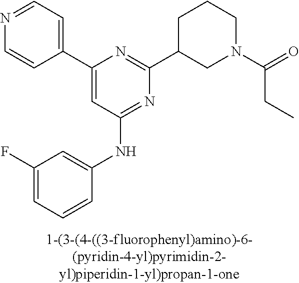 [SMILES: CCC(=O)N1CCCC(c2nc(Nc3cccc(F)c3)cc(-c3ccncc3)n2)C1] | ¹H-NMR (400 MHz, DMSO-d6) mixture of rotamers δ 10.06 (d, J = 10.8 Hz, 1H), 8.76 (d, J = 4.9 Hz, 2H), 7.98 (d, J = 5.1 Hz, 2H), 7.90 (dd, J = 20.5, 12.3 Hz, 1H), 7.47-7.33 (m, 2H), 7.23 (d, J = 3.2 Hz, 1H), 6.86 (t, J = 8.4 Hz, 1H), 4.77 (d, J = 12.5 Hz, 0.5H), 4.23 (dd, J = 28.0, 13.1 Hz, 1H), 3.90 (d, J = 13.7 Hz, 0.5 H), 3.45 (dd, J = 13.3, 10.5 Hz, 0.5H), 3.07 (t, J = 12.6 Hz, 0.5H), 2.98-2.65 (m, 2H), 2.42-2.31 (m, 2H), 2.23 (s, 1H), 1.98- 1.71 (m, 2H), 1.66-1.39 (m, 1H), 1.05-0.93 (m, 3H); LCMS (Method D): t_{R} 3.48 min, 100%, MS (ESI) 406.2 (M + H)⁺ |
| 00004 | 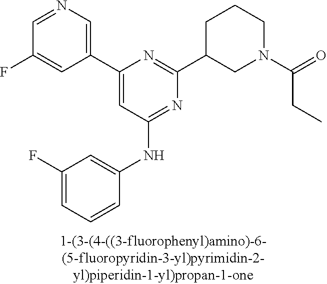 [SMILES: CCC(=O)N1CCCC(c2nc(Nc3cccc(F)c3)cc(-c3cncc(F)c3)n2)C1] | ¹H-NMR (400 MHz, DMSO-d6) mixture of rotamers δ 10.06 (d, J = 11.2 Hz, 1H), 9.09 (s, 1H), 8.74 (s, 1H), 8.28 (d, J = 9.8 Hz, 1H), 7.88 (dd, J = 19.9, 12.2 Hz, 1H), 7.47-7.33 (m, 2H), 7.22 (s, 1H), 6.86 (s, 1H), 4.73 (d, J = 13.0 Hz, 0.5H), 4.22 (dd, J = 29.6, 13.3 Hz, 1H), 3.88 (d, J = 13.4 Hz, 0.5H), 3.51-3.40 (m, 0.5H), 3.07 (t, J = 12.3 Hz, 0.5H), 2.93 (t, J = 11.6 Hz, 1H), 2.85-2.70 (m, 1H), 2.41-2.32 (m, 2H), 2.23 (s, 1H), 1.95-1.70 (m, 2H), 1.65-1.37 (m, 1H), 1.05-0.91 (m, 3H) ); LCMS (Method D): t_{R} 3.67 min, 100%, MS (ESI) 424.2 (M + H)⁺ |
| 00005 | 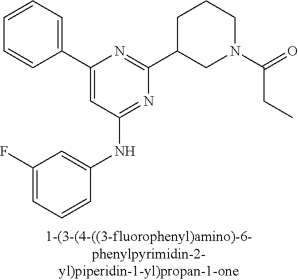 [SMILES: CCC(=O)N1CCCC(c2nc(Nc3cccc(F)c3)cc(-c3ccccc3)n2)C1] | ¹H-NMR (400 MHz, DMSO-d6) mixture of rotamers δ 9.92 (d, J = 10.5 Hz, 2H), 8.11-8.00 (m, 3H), 7.91 (dd, J = 20.8, 12.4 Hz, 2H), 7.60-7.48 (m, 5H), 7.45-7.31 (m, 3H), 7.13 (d, J = 2.6 Hz, 2H), 6.90-6.77 (m, 1H), 4.79 (d, J = 12.5 Hz, 0.5H), 4.23 (dd, J = 29.2, 13.3 Hz, 2H), 3.91 (d, J = 12.9 Hz, 0.5H), 3.51-3.39 (m, 0.5H), 3.05 (t, J = 13.0 Hz, 0.5H), 2.97-2.68 (m, 3H), 2.43-2.31 (m, 4H), 2.29-2.18 (m, 2H), 1.97-1.73 (m, 3H), 1.68- 1.39 (m, 2H), 1.07-0.93 (m, 5H); LCMS (Method D): t_{R} 3.99 min, 100%, MS (ESI) 405.2 (M + H)⁺ |
| 00006 | 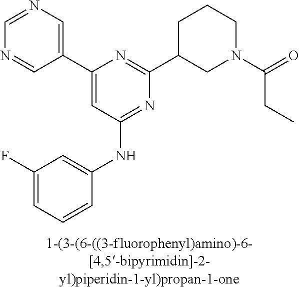 [SMILES: CCC(=O)N1CCCC(c2nc(Nc3cccc(F)c3)cc(-c3cncnc3)n2)C1] | ¹H-NMR (400 MHz, DMSO-d6) mixture of rotamers δ 10.09 (d, J = 11.4 Hz, 1H), 9.38 (d, J = 5.0 Hz, 2H), 9.33 (d, J = 1.9 Hz, 1H), 7.95-7.81 (m, 1H), 7.48-7.33 (m, 2H), 7.23 (d, J = 3.9 Hz, 1H), 6.86 (t, J = 8.5 Hz, 1H), 4.73 (d, J = 12.2 Hz, 0.5H), 4.23 (dd, J = 25.3, 12.8 Hz, 1H), 3.89 (d, J = 13.6 Hz, 0.5H), 3.45 (dd, J = 13.5, 10.2 Hz, 0.5H), 3.07 (t, J = 12.8 Hz, 0.5H), 3.01- 2.87 (m, 1H), 2.86-2.71 (m, 1H), 2.45-2.31 (m, 2H), 2.29-2.16 (m, 1H), 2.01-1.69 (m, 2H), 1.65-1.39 (m, 1H), 1.07-0.95 (m, 3H); LCMS (Method D): t_{R} 3.33 min, 100%, MS (ESI) 407.2 (M + H)⁺ |
| 00007 | 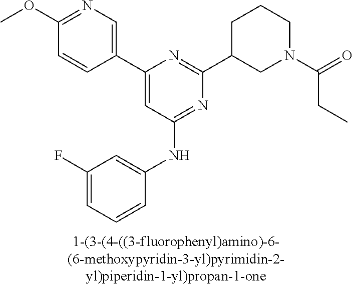 [SMILES: CCC(=O)N1CCCC(c2nc(Nc3cccc(F)c3)cc(-c3ccc(OC)nc3)n2)C1] | ¹H-NMR (400 MHz, DMSO-d6) mixture of rotamers δ 9.90 (d, J = 9.4 Hz, 1H), 8.85 (dd, J = 6.1, 2.4 Hz, 1H), 8.33 (dd, J = 8.7, 2.5 Hz, 1H), 7.89 (dd, J = 20.6, 12.2 Hz, 1H), 7.69-7.50 (m, 0.5H), 7.47-7.30 (m, 2H), 7.07 (d, J = 2.2 Hz, 1H), 6.98 (d, J = 8.6 Hz, 1H), 6.88-6.74 (m, 1H), 4.75 (d, J = 12.6 Hz, 1H), 4.22 (dd, J = 28.8, 13.2 Hz, 1H), 3.94 (s, 4H), 3.54-3.37 (m, 0.5H), 3.05 (t, J = 12.7 Hz, 0.5H), 2.88 (dd, J = 19.8, 8.6 Hz, 1H), 2.75 (t, J = 11.7 Hz, 1H), 2.39 (dt, J = 12.4, 7.1 Hz, 2H), 2.22 (s, 1H), 1.98-1.70 (m, 2H), 1.51 (dd, J = 41.5, 12.7 Hz, 1H), 1.00 (q, J = 7.6 Hz, 3H); LCMS (Method D): t_{R} 3.83 min, 100%, MS (ESI) 436.2 (M + H)⁺ |
| 00008 | 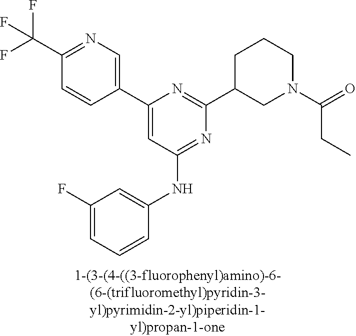 [SMILES: CCC(=O)N1CCCC(c2nc(Nc3cccc(F)c3)cc(-c3ccc(C(F)(F)F)nc3)n2)C1] | ¹H-NMR (400 MHz, DMSO-d6) mixture of rotamers δ 10.09 (d, J = 9.6 Hz, 1H), 9.36 (d, 1H), 8.67 (d, J = 8.3 Hz, 1H), 8.10 (d, J = 8.2 Hz, 1H), 7.89 (dd, J = 20.0, 12.1 Hz, 1H), 7.49-7.33 (m, 2H), 7.26 (s, 1H), 6.92-6.80 (m, 1H), 4.34-4.14 (m, 0.5H), 3.96-3.82 (m, 1H), 3.50-3.39 (m, 0.5H), 3.12-2.70 (m, 2H), 2.40-2.30 (m, 2H), 2.30- 2.15 (m, 1H), 1.98-1.72 (m, 2H), 1.67-1.37 (m, 1H), 1.00 (q, J = 7.7 Hz, 3H); LCMS (Method D): t_{R} 3.99 min, 100%, MS (ESI) 474.2 (M + H)⁺ |
| 00009 | 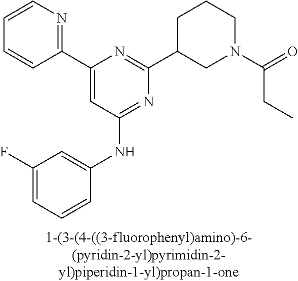 [SMILES: CCC(=O)N1CCCC(c2nc(Nc3cccc(F)c3)cc(-c3ccccn3)n2)C1] | ¹H-NMR (400 MHz, DMSO-d6) mixture of rotamers δ 10.01 (d, J = 10.3 Hz, 1H), 8.72 (d, J = 4.7 Hz, 1H), 8.41 (d, J = 7.9 Hz, 1H), 8.00 (t, J = 7.7 Hz, 1H), 7.91 (dd, J = 22.0, 12.2 Hz, 1H), 7.72 (d, J = 3.0 Hz, 1H), 7.58-7.50 (m, 1H), 7.46-7.32 (m, 2H), 6.84 (s, 1H), 4.79 (d, J = 12.7 Hz, 0.5H), 4.26 (dd, J = 25.1, 12.9 Hz, 1H), 3.91 (d, J = 13.8 Hz, 0.5H), 3.50-3.41 (m, 0.5H), 3.11-3.00 (m, 0.5H), 3.00- 2.65 (m, 2H), 2.44-2.32 (m, 2H), 2.31-2.19 (m, 1H), 2.00-1.73 (m, 2H), 1.67-1.41 (m, 1H), 1.08-0.93 (m, 3H); LCMS (Method D): t_{R} 3.68 min, 100%, MS (ESI) 406.2 (M + H)⁺ |
| 00010 | 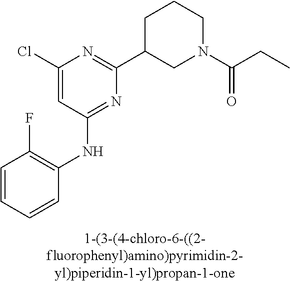 [SMILES: CCC(=O)N1CCCC(c2nc(Cl)cc(Nc3ccccc3F)n2)C1] | ¹H-NMR (400 MHz, chloroform-d) a mixture of rotamers δ 7.90-7.74 (m, 1H), 7.24-7.11 (m, 3H), 6.80 (s, 1H), 6.53 (d, J = 7.8 Hz, 1H), 4.91 (d, J = 10.7 Hz, 0.5H), 4.50 (d, J = 13.2 Hz, 0.5H), 4.05 (d, J = 14.2 Hz, 0.5H), 3.88 (d, J = 13.6 Hz, 0.5H), 3.43 (dd, J = 13.4, 10.6 Hz, 0.5H), 3.05 (t, J = 12.1 Hz, 0.5H), 2.94-2.81 (m, 1H), 2.81-2.69 (m, 0.5H), 2.47-2.34 (m, 2H), 2.28-2.15 (m, 1H), 1.93-1.70 (m, 2H), 1.30-1.21 (m, 0.5H), 1.21- 1.11 (m, 3H); LCMS (Method D): t_{R} 3.47 min, 100%, MS (ESI) 363.2 (M + H)⁺ |
| 00011 | 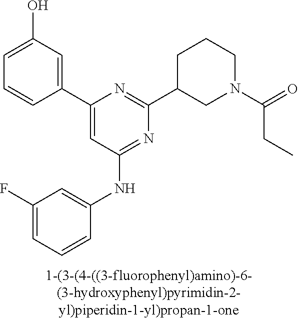 [SMILES: CCC(=O)N1CCCC(c2nc(Nc3cccc(F)c3)cc(-c3cccc(O)c3)n2)C1] | ¹H-NMR (400 MHz, DMSO-d6) mixture of rotamers δ 9.88 (d, J = 10.1 Hz, 1H), 9.70 (s, 1H), 7.91 (dd, J = 20.6, 12.2 Hz, 1H), 7.53-7.29 (m, 5H), 7.06 (s, 1H), 6.91 (d, 1H), 6.87-6.78 (m, 1H), 4.80 (d, J = 11.8 Hz, 0.5H), 4.24 (dd, J = 35.2, 13.8 Hz, 1H), 3.91 (d, J = 13.4 Hz, 0.5H), 3.49-3.38 (m, 1H), 3.04 (t, J = 12.3 Hz, 0.5H), 2.95- 2.86 (m, 0.5H), 2.88-2.64 (m, 2H), 2.46-2.31 (m, 3H), 2.29-2.19 (m, 1H), 1.98-1.68 (m, 2H), 1.68-1.38 (m, 1H), 1.07-0.94 (m, 3H); LCMS (Method D): t_{R} 3.58 min, 100%, MS (ESI) 421.2 (M + H)⁺ |
| 00012 | 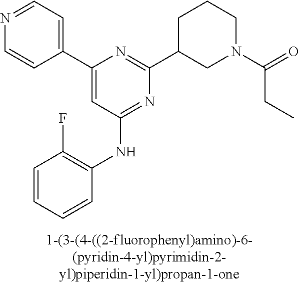 [SMILES: CCC(=O)N1CCCC(c2nc(Nc3ccccc3F)cc(-c3ccncc3)n2)C1] | ¹H-NMR (400 MHz, DMSO-d6) mixture of rotamers δ 9.54 (d, J = 11.1 Hz, 1H), 8.79-8.70 (m, 2H), 8.17-8.04 (m, 1H), 7.95 (d, J = 5.0 Hz, 2H), 7.38- 7.20 (m, 3H), 7.20-7.10 (m, 1H), 4.75-4.65 (m, 0.5H), 4.25-4.16 (m, 0.5H), 4.15-4.05 (m, 0.5H), 3.87 (d, J = 13.7 Hz, 0.5H), 3.48-3.39 (m, 0.5H), 3.08-2.97 (m, 0.5H), 2.92- 2.63 (m, 2H), 2.40-2.29 (m, 2H), 2.22-2.12 (m, 1H), 1.98-1.68 (m, 2H), 1.63-1.20 (m, 2H), 1.04-0.91 (m, 3H) ); LCMS (Method B): t_{R} 2.84 min, 100%, MS (ESI) 406.2 (M + H)⁺ |
| 00013 | 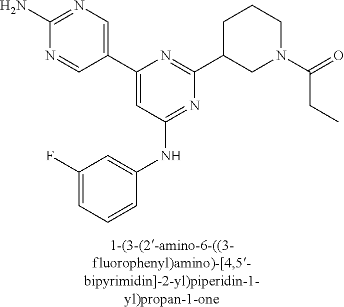 [SMILES: CCC(=O)N1CCCC(c2nc(Nc3cccc(F)c3)cc(-c3cnc(N)nc3)n2)C1] | ¹H-NMR (400 MHz, DMSO-d6) mixture of rotamers δ 9.83 (d, J = 10.9 Hz, 1H), 8.87 (d, J = 3.0 Hz, 2H), 7.87 (dd, J = 20.5, 12.3 Hz, 1H), 7.68-7.52 (m, 1H), 7.45-7.31 (m, 2H), 7.18 (s, 2H), 6.98 (d, J = 2.8 Hz, 1H), 6.82 (t, J = 8.6 Hz, 1H), 4.73 (d, J = 12.1 Hz, 0.5H), 4.21 (dd, J = 35.5, 13.5 Hz, 1H), 3.89 (d, J = 13.9 Hz, 0.5H), 3.42 (dd, J = 13.5, 10.2 Hz, 0.5H), 3.04 (t, J = 12.7 Hz, 0.5H), 2.94-2.65 (m, 2H), 2.43-2.30 (m, 3H), 2.19 (s, 1H), 1.81 (td, J = 24.2, 22.7, 12.9 Hz, 2H), 1.58- 1.38 (m, 1H), 1.00 (q, J = 7.3 Hz, 3H); LCMS (Method B): t_{R} 3.22 min, 100%, MS (ESI) 422.2 (M + H)⁺ |
| 00014 | 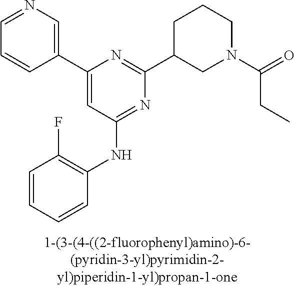 [SMILES: CCC(=O)N1CCCC(c2nc(Nc3ccccc3F)cc(-c3cccnc3)n2)C1] | ¹H-NMR (400 MHz, DMSO-d6) mixture of rotamers δ 9.46 (d, J = 11.0 Hz, 1H), 9.18 (s, 1H), 8.70 (d, J = 4.7 Hz, 1H), 8.40-8.32 (m, 1H), 8.17-8.06 (m, 1H), 7.62-7.52 (m, 1H), 7.35- 7.27 (m, 2H), 7.26-7.20 (m, 1H), 7.20-7.11 (m, 1H), 4.71 (d, J = 12.5 Hz, 0.5H), 4.21 (d, J = 12.6 Hz, 0.5H), 4.10 (d, J = 13.6 Hz, 0.5H), 3.87 (d, J = 14.1 Hz, 0.5H), 3.49-3.39 (m, 0.5H), 3.03 (t, J = 12.6 Hz, 0.5H), 2.92- 2.62 (m, 2H), 2.36-2.29 (m, 2H), 2.22-2.10 (m, 1H), 1.94-1.66 (m, 2H), 1.61-1.35 (m, 1H), 1.02-0.94 (m, 3H); LCMS (Method B): t_{R} 2.92 min, 100%, MS (ESI) 406.2 (M + H)⁺ |
| 00015 | 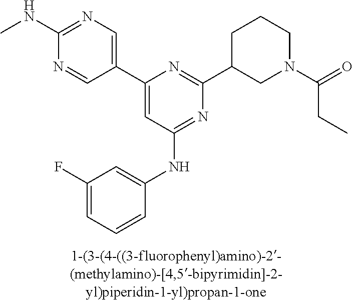 [SMILES: CCC(=O)N1CCCC(c2nc(Nc3cccc(F)c3)cc(-c3cnc(NC)nc3)n2)C1] | ¹H-NMR (400 MHz, DMSO-d6) mixture of rotamers δ 9.83 (d, J = 9.3 Hz, 1H), 8.92 (s, 2H), 7.87 (dd, J = 20.8, 12.2 Hz, 1H), 7.71-7.61 (m, 1H), 7.49- 7.29 (m, 2H), 6.97 (s, 1H), 6.82 (t, J = 8.5 Hz, 1H), 4.73 (d, J = 12.4 Hz, 0.5H), 4.21 (dd, J = 30.7, 13.3 Hz, 1H), 3.89 (d, J = 13.8 Hz, 0.5H), 3.42 (dd, J = 13.4, 10.3 Hz, 1H), 3.05 (t, J = 12.7 Hz, 0.5H), 2.93-2.82 (m, 4H), 2.81-2.64 (m, 1H), 2.45-2.30 (m, 2H), 2.21 (s, 1H), 1.94-1.70 (m, 2H), 1.61-1.37 (m, 1H), 1.05-0.95 (m, 3H); LCMS (Method D): t_{R} 3.39 min, 100%, MS (ESI) 436.2 (M + H)⁺ |
| 00016 | 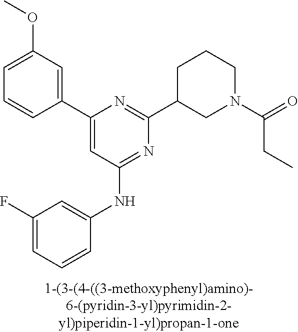 | ¹H-NMR (400 MHz, DMSO-d6) mixture of rotamers δ 9.76 (d, J = 6.5 Hz, 1H), 9.20 (d, J = 3.0 Hz, 1H), 8.70 (d, J = 4.7 Hz, 1H), 8.37 (dt, J = 8.0, 2.0 Hz, 1H), 7.64-7.52 (m, 2H), 7.30-7.17 (m, 2H), 7.15 (s, 1H), 6.67-6.58 (m, 1H), 4.78 (d, J = 12.8 Hz, 0.5H), 4.26 (d, J = 12.9 Hz, 0.5H), 4.17 (d, J = 13.9 Hz, 0.5H), 3.90 (d, J = 13.6 Hz, 0.5H), 3.78 (s, 3H), 3.46 (dd, J = 13.4, 10.2 Hz, 0.5H), 3.03 (t, J = 12.8 Hz, 0.5H), 2.97-2.65 (m, 2H), 2.43-2.30 (m, 2H), 2.22 (d, J = 12.7 Hz, 1H), 1.99-1.71 (m, 2H), 1.66-1.39 (m, 1H), 1.07-0.93 (m, 3H) ); LCMS (Method D): t_{R} 3.32 min, 100%, MS (ESI) 418.2 (M + H)⁺ |
| 00017 | 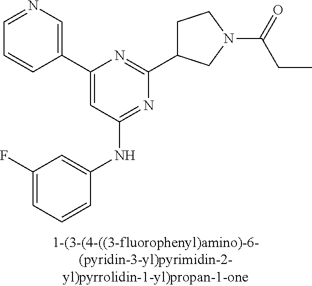 [SMILES: CCC(=O)N1CCC(c2nc(Nc3cccc(F)c3)cc(-c3cccnc3)n2)C1] | ¹H-NMR (400 MHz, DMSO-d6) δ 10.03 (d, J = 4.6 Hz, 1H), 9.21 (s, 1H), 8.82-8.66 (m, 1H), 8.41 (d, J = 8.0 Hz, 1H), 7.87-7.75 (m, 1H), 7.67- 7.55 (m, 1H), 7.49-7.41 (m, 1H), 7.41-7.31 (m, 1H), 7.19 (s, 1H), 6.93- 6.80 (m, 1H), 3.95-3.50 (m, 5H), 2.46-2.14 (m, 4H), 1.00 (m, 3H); LCMS (Method D): t_{R} 3.13 min, 100%, MS (ESI) 392.1 (M + H)⁺ |
| 00018 | 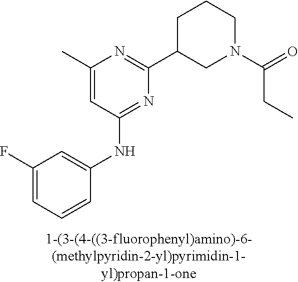 [SMILES: CCC(=O)N1CCCC(c2nc(C)cc(Nc3cccc(F)c3)n2)C1] | ¹H-NMR (400 MHz, DMSO-d6) mixture of rotamers δ 9.67 (d, J = 9.9 Hz, 1H), 7.93-7.75 (m, 1H), 7.42-7.28 (m, 2H), 6.80 (d, J = 7.5 Hz, 1H), 6.51 (d, J = 2.2 Hz, 1H), 4.72 (d, J = 11.7 Hz, 0.5H), 4.24 (d, J = 13.3 Hz, 0.5H), 4.07 (d, J = 14.1 Hz, 0.5H), 3.89 (d, J = 13.8 Hz, 0.5H), 3.38 (s, 0.5H), 2.99 (t, J = 13.2 Hz, 0.5H), 2.85-2.60 (m, 2H), 2.42-2.31 (m, 2H), 2.30 (s, 3H), 2.13 (d, J = 13.4 Hz, 1H), 1.89-1.65 (m, 2H), 1.62-1.32 (m, 1H), 1.05- 0.95 (m, 3H); LCMS (Method D): t_{R} 3.25 min, 100%, MS (ESI) 343.2 (M + H)⁺ |
| 00019 | 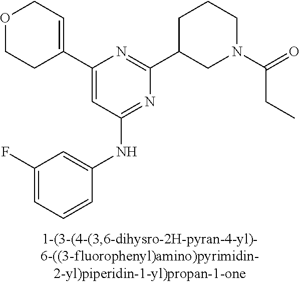 [SMILES: CCC(=O)N1CCCC(c2nc(Nc3cccc(F)c3)cc(C3=CCOCC3)n2)C1] | ¹H-NMR (400 MHz, DMSO-d6) mixture of rotamers δ 9.83 (d, J = 11.0 Hz, 1H), 7.89 (dd, J = 22.2, 12.3 Hz, 1H), 7.44-7.29 (m, 2H), 7.03 (s, 1H), 6.87- 6.76 (m, 1H), 6.63 (d, J = 1.8 Hz, 1H), 4.73 (d, 0.5H), 4.33-4.28 (m, 2H), 4.26 (d, J = 12.2 Hz, 0.5H), 4.14 (d, J = 13.8 Hz, 0.5H), 3.89 (d, J = 13.5 Hz, 0.5H), 3.83 (t, J = 5.4 Hz, 2H), 3.41-3.35 (m, 0.5H), 3.01 (t, J = 12.8 Hz, 0.5H), 2.89-2.63 (m, 2H), 2.43 (s, 2H), 2.36 (q, J = 7.5 Hz, 3H), 2.17 (d, J = 11.4 Hz, 1H), 1.93-1.69 (m, 3H), 1.63-1.36 (m, 1H), 1.05- 0.95 (m, 4H); LCMS (Method D): t_{R} 3.52 min, 100%, MS (ESI) 411.2 (M + H)⁺ |
| 00020 | 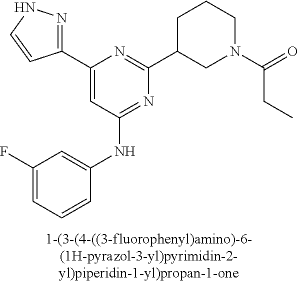 [SMILES: CCC(=O)N1CCCC(c2nc(Nc3cccc(F)c3)cc(-c3cc[nH]n3)n2)C1] | ¹H-NMR (400 MHz, DMSO-d6) mixture of rotamers δ 13.21 (s, 1H), 9.82 (d, J = 9.4 Hz, 1H), 7.96-7.78 (m, 2H), 7.44-7.32 (m, 2H), 7.26 (s, 1H), 6.89- 6.76 (m, 2H), 4.75 (d, J = 12.5 Hz, 0.5H), 4.19 (dd, J = 28.2, 13.2 Hz, 1H), 3.90 (d, J = 13.9 Hz, 0.5H), 3.51- 3.40 (m, 0.5H), 3.04 (t, J = 12.8 Hz, 0.5H), 2.94-2.65 (m, 2H), 2.44-2.31 (m, 2H), 2.21 (s, 1H), 1.97-1.70 (m, 2H), 1.66-1.38 (m, 1H), 1.00 (q, J = 7.5 Hz, 3H); LCMS (Method D): t_{R} 3.27 min, 100%, MS (ESI) 395.2 (M + H)⁺ |
| 00021 | 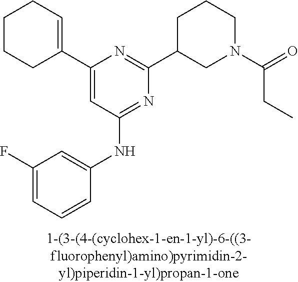 [SMILES: CCC(=O)N1CCCC(c2nc(Nc3cccc(F)c3)cc(C3=CCCCC3)n2)C1] | ¹H-NMR (400 MHz, DMSO-d6) mixture of rotamers δ 9.74 (d, J = 10.5 Hz, 1H), 7.89 (dd, J = 22.1, 12.3 Hz, 1H), 7.44-7.27 (m, 2H), 7.04 (s, 1H), 6.79 (t, J = 8.4 Hz, 1H), 6.61 (d, J = 2.0 Hz, 1H), 4.72 (d, J = 12.3 Hz, 0.5H), 4.30- 4.07 (m, 1H), 3.89 (d, J = 13.8 Hz, 0.5H), 3.41-3.35 (m, 0.5H), 3.00 (t, J = 12.6 Hz, 0.5H), 2.88-2.59 (m, 2H), 2.35 (q, J = 7.6 Hz, 4H), 2.24 (s, 2H), 2.16 (d, J = 12.7 Hz, 1H), 1.94-1.68 (m, 4H), 1.65-1.37 (m, 3H), 1.00 (td, J = 7.3, 5.2 Hz, 3H); LCMS (Method D): t_{R} 4.12 min, 100%, MS (ESI) 409.2 (M + H)⁺ |
| 00022 | 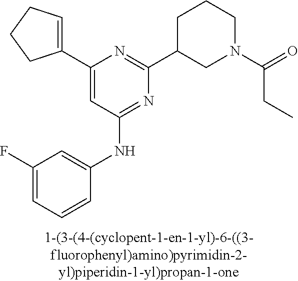 [SMILES: CCC(=O)N1CCCC(c2nc(Nc3cccc(F)c3)cc(C3=CCCC3)n2)C1] | ¹H-NMR (400 MHz, DMSO-d6) mixture of rotamers δ 9.78 (d, J = 10.2 Hz, 1H), 7.89 (dd, J = 21.6, 12.2 Hz, 1H), 7.42-7.28 (m, 2H), 6.80 (s, 2H), 6.56 (s, 1H), 4.71 (d, J = 12.5 Hz, 0.5H), 4.17 (dd, J = 42.8, 13.3 Hz, 1H), 3.89 (d, J = 13.1 Hz, 0.5H), 3.45-3.36 (m, 0.5H), 3.01 (t, J = 12.6 Hz, 0.5H), 2.88- 2.66 (m, 2H), 2.67-2.59 (m, 2H), 2.53 (s, 2H), 2.41-2.31 (m, 2H), 2.15 (s, 1H), 2.05-1.95 (m, 2H), 1.85- 1.68 (m, 2H), 1.61-1.36 (m, 1H), 1.00 (q, J = 7.1 Hz, 3H); LCMS (Method D): t_{R} 3.95 min, 100%, MS (ESI) 395.2 (M + H)⁺ |
| 00023 | 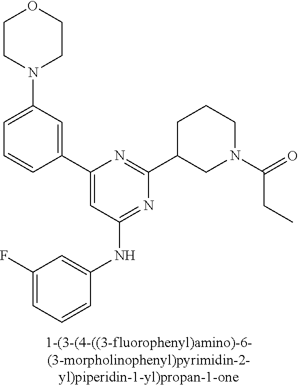 [SMILES: CCC(=O)N1CCCC(c2nc(Nc3cccc(F)c3)cc(-c3cccc(N4CCOCC4)c3)n2)C1] | ¹H-NMR (400 MHz, DMSO-d6) mixture of rotamers δ 9.87 (d, J = 10.4 Hz, 1H), 7.92 (dd, J = 21.6, 12.3 Hz, 1H), 7.59 (d, J = 8.4 Hz, 1H), 7.47 (d, J = 7.7 Hz, 1H), 7.43-7.31 (m, 3H), 7.18- 7.08 (m, 2H), 6.82 (t, J = 8.5 Hz, 1H), 4.75 (d, J = 12.7 Hz, 0.5H), 4.21 (t, J = 15.6 Hz, 1H), 3.90 (d, J = 13.6 Hz, 0.5H), 3.78 (t, J = 4.7 Hz, 4H), 3.46 (dd, J = 13.4, 10.1 Hz, 0.5H), 3.19 (t, J = 4.8 Hz, 4H), 3.05 (t, J = 12.8 Hz, 0.5H), 2.99-2.70 (m, 2H), 2.46-2.33 (m, 2H), 2.22 (s, 1H), 1.99- 1.69 (m, 2H), 1.64-1.39 (m, 1H), 1.00 (q, J = 7.8 Hz, 3H); LCMS (Method D): t_{R} 3.79 min, 100%, MS (ESI) 490.3 (M + H)⁺ |
| 00024 | 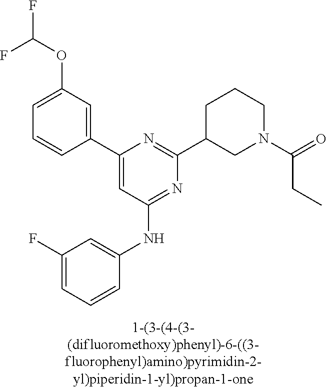 [SMILES: CCC(=O)N1CCCC(c2nc(Nc3cccc(F)c3)cc(-c3cccc(OC(F)F)c3)n2)C1] | ¹H-NMR (400 MHz, DMSO-d6) mixture of rotamers δ 9.96 (d, J = 10.9 Hz, 1H), 7.97-7.85 (m, 2H), 7.84 (s, 1H), 7.61 (t, J = 8.0 Hz, 1H), 7.56-7.16 (m, 5H), 7.16-7.12 (m, 1H), 6.84 (t, J = 8.5 Hz, 1H), 4.76 (d, J = 12.0 Hz, 0.5H), 4.30-4.15 (m, 1H), 3.89 (d, J = 13.5 Hz, 0.5H), 3.45 (dd, J = 13.4, 10.2 Hz, 0.5H), 3.06 (t, J = 12.8 Hz, 0.5H), 2.99-2.63 (m, 2H), 2.45-2.31 (m, 3H), 2.23 (s, 1H), 1.84 (td, J = 26.4, 24.0, 13.3 Hz, 2H), 1.66-1.39 (m, 1H), 1.00 (q, J = 7.6 Hz, 3H); LCMS (Method D): t_{R} 3.98 min, 100%, MS (ESI) 471.2 (M + H)⁺ |
| 00025 | 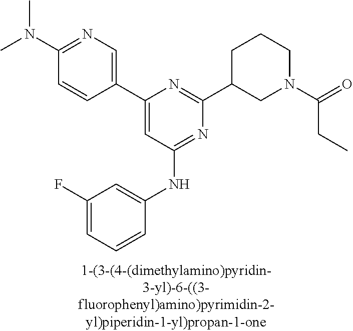 [SMILES: CCC(=O)N1CCCC(c2nc(Nc3cccc(F)c3)cc(-c3ccc(N(C)C)nc3)n2)C1] | ¹H-NMR (400 MHz, DMSO-d6) mixture of rotamers δ 9.78 (d, J = 9.8 Hz, 1H), 8.80 (dd, J = 6.2, 2.3 Hz, 1H), 8.13 (dd, J = 9.0, 2.4 Hz, 1H), 7.89 (dd, J = 21.4, 12.3 Hz, 1H), 7.44-7.29 (m, 2H), 6.97 (d, J = 2.0 Hz, 1H), 6.86- 6.70 (m, 2H), 4.76 (d, J = 12.6 Hz, 0.5H), 4.22 (dd, J = 29.5, 13.8 Hz, 1H), 3.90 (d, J = 14.4 Hz, 0.5H), 3.49- 3.37 (m, 1H), 3.11 (s, 6H), 3.04 (t, J = 12.5 Hz, 0.5H), 2.90-2.61 (m, 2H), 2.44-2.31 (m, 2H), 2.27-2.13 (m, 1H), 1.97-1.72 (m, 2H), 1.66-1.40 (m, 1H), 1.00 (q, J = 7.5 Hz, 3H); LCMS (Method D): t_{R} 3.72 min, 100%, MS (ESI) 449.2 (M + H)⁺ |
| 00026 | 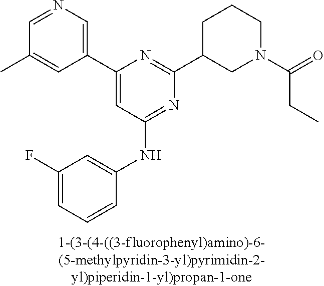 [SMILES: CCC(=O)N1CCCC(c2nc(Nc3cccc(F)c3)cc(-c3cncc(C)c3)n2)C1] | ¹H-NMR (400 MHz, DMSO-d6) mixture of rotamers δ 9.96 (d, J = 9.3 Hz, 1H), 9.00 (d, J = 2.4 Hz, 1H), 8.55 (s, 1H), 8.20 (S, 1H), 7.89 (dd, J = 19.7, 12.2 Hz, 1H), 7.49-7.32 (m, 3H), 7.16 (d, J = 2.0 Hz, 1H), 6.83 (d, J = 8.9 Hz, 1H), 4.76 (d, J = 12.3 Hz, 0.5H), 4.22 (dd, J = 31.1, 12.4 Hz, 1H), 3.90 (d, J = 13.1 Hz, 0.5H), 3.51-3.41 (m, 0.5H), 3.06 (t, J = 12.9 Hz, 0.5H), 2.99- 2.65 (m, 2H), 2.44-2.32 (m, 5H), 2.21 (s, 1H), 2.00-1.71 (m, 2H), 1.64- 1.39 (m, 1H), 1.00 (q, J = 7.8 Hz, 3H); ); LCMS (Method D): t_{R} 3.53 min, 100%, MS (ESI) 420.2 (M + H)⁺ |
| 00027 | 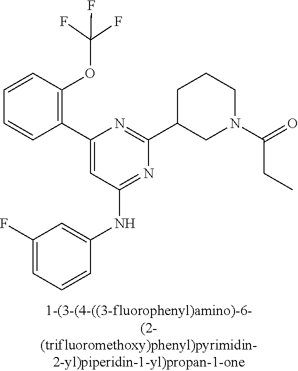 [SMILES: CCC(=O)N1CCCC(c2nc(Nc3cccc(F)c3)cc(-c3ccccc3OC(F)(F)F)n2)C1] | ¹H-NMR (400 MHz, DMSO-d6) mixture of rotamers δ 9.99 (d, J = 9.9 Hz, 1H), 7.97-7.82 (m, 2H), 7.68-7.61 (m, 1H), 7.60-7.49 (m, 3H), 7.39 (dq, J = 15.5, 8.2 Hz, 2H), 7.00 (d, J = 3.0 Hz, 1H), 6.91-6.79 (m, 1H), 4.76 (d, J = 12.1 Hz, 0.5H), 4.33-4.09 (m, 1H), 3.89 (d, J = 13.4 Hz, 0.5H), 3.40 (dd, J = 13.5, 10.2 Hz, 0.5H), 3.09-2.98 (m, 0.5H), 2.98-2.66 (m, 2H), 2.36 (m, 2H), 2.21 (d, J = 12.7 Hz, 1H), 1.99- 1.69 (m, 2H), 1.67-1.39 (m, 1H), 0.99 (m, 3H); LCMS (Method D): t_{R} 4.03 min, 100%, MS (ESI) 489.1 (M + H)⁺ |
| 00028 | 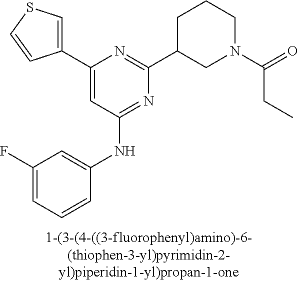 [SMILES: CCC(=O)N1CCCC(c2nc(Nc3cccc(F)c3)cc(-c3ccsc3)n2)C1] | ¹H-NMR (400 MHz, DMSO-d6) mixture of rotamers δ 9.87 (d, J = 10.9 Hz, 1H), 8.25 (s, 1H), 7.89 (dd, J = 21.5, 12.2 Hz, 1H), 7.67 (dd, J = 15.3, 4.3 Hz, 2H), 7.44-7.31 (m, 2H), 6.98 (d, J = 2.6 Hz, 1H), 6.82 (t, J = 8.4 Hz, 1H), 4.73 (d, J = 13.0 Hz, 0.5H), 4.21 (dd, J = 31.6, 13.0 Hz, 1H), 3.89 (d, J = 13.6 Hz, 0.5H), 3.49-3.39 (m, 0.5H), 3.13-2.99 (m, 0.5H), 2.95- 2.65 (m, 2H), 2.44-2.30 (m, 2H), 2.21 (s, 1H), 1.99-1.72 (m, 2H), 1.62- 1.42 (m, 1H), 1.00 (q, J = 7.4 Hz, 3H); LCMS (Method D): t_{R} 3.82 min, 100%, MS (ESI) 411.1 (M + H)⁺ |
| 00029 | 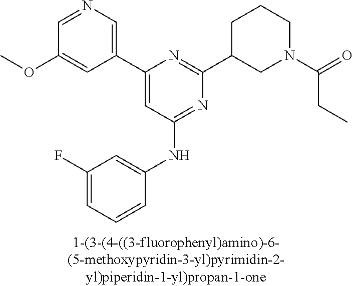 [SMILES: CCC(=O)N1CCCC(c2nc(Nc3cccc(F)c3)cc(-c3cncc(OC)c3)n2)C1] | ¹H-NMR (400 MHz, DMSO-d6) mixture of rotamers δ 9.98 (d, J = 10.3 Hz, 1H), 8.84-8.76 (m, 1H), 8.43 (s, 1H), 7.96-7.83 (m, 2H), 7.47-7.32 (m, 2H), 7.18 (d, J = 3.7 Hz, 1H), 6.85 (t, J = 8.5 Hz, 1H), 4.73 (d, J = 12.7 Hz, 0.5H), 4.21 (dd, J = 17.3 Hz, 1H), 3.98- 3.84 (m, 3.5H), 3.46 (dd, J = 13.4, 10.2 Hz, 0.5H), 3.07 (t, J = 12.4 Hz, 0.5H), 3.00-2.87 (m, 1H), 2.87-2.72 (m, 1H), 2.44-2.31 (m, 2H), 2.23 (s, 1H), 1.99-1.71 (m, 2H), 1.66-1.38 (m, 1H), 0.99 (q, J = 7.7 Hz, 3H); LCMS (Method D): t_{R} 3.50 min, 100%, MS (ESI) 436.2 (M + H)⁺ |
| 00030 | 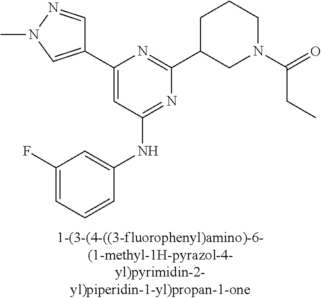 [SMILES: CCC(=O)N1CCCC(c2nc(Nc3cccc(F)c3)cc(-c3cnn(C)c3)n2)C1] | ¹H-NMR (400 MHz, DMSO-d6) mixture of rotamers δ 9.75 (d, J = 9.8 Hz, 1H), 8.30 (d, J = 2.4 Hz, 1H), 7.95 (d, J = 4.5 Hz, 1H), 7.85 (dd, J = 20.6, 12.3 Hz, 1H), 7.42-7.28 (m, 2H), 6.86- 6.74 (m, 2H), 4.73 (d, J = 12.0 Hz, 0.5H), 4.25 (d, J = 13.1 Hz, 0.5H), 4.14 (d, J = 13.6 Hz, 0.5H), 3.96- 3.85 (m, 3.5H), 3.41 (dd, J = 13.4, 10.3 Hz, 0.5H), 3.02 (t, J = 12.5 Hz, 0.5H), 2.91-2.61 (m, 2H), 2.44-2.31 (m, 2H), 2.25-2.13 (m, 1H), 1.95- 1.66 (m, 2H), 1.62-1.37 (m, 1H), 1.00 (q, J = 7.4 Hz, 3H); LCMS (Method D): t_{R} 3.30 min, 100%, MS (ESI) 409.2 (M + H)⁺ |
| 00031 | 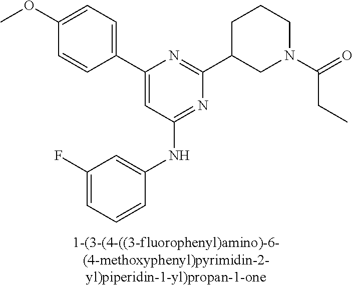 [SMILES: CCC(=O)N1CCCC(c2nc(Nc3cccc(F)c3)cc(-c3ccc(OC)cc3)n2)C1] | ¹H-NMR (400 MHz, DMSO-d₆) mixture of rotamers δ 9.85 (d, J = 10.5 Hz, 1H), 8.08-7.98 (m, 2H), 7.97-7.84 (m, 1H), 7.44-7.32 (m, 2H), 7.15- 7.02 (m, 3H), 6.87-6.77 (m, 1H), 4.85-4.72 (m, 0.5H), 4.34-4.13 (m, 1H), 3.91 (m, 0.5H), 3.84 (s, 3H), 3.54- 3.38 (m, 0.5H), 3.09-3.00 (m, 0.5H), 2.96-2.68 (m, 2H), 2.43-2.30 (m, 2H), 2.29-2.16 (m, 1H), 1.97-1.71 (m, 2H), 1.66-1.37 (m, 1H), 1.00 (q, J = 7.7 Hz, 3H); LCMS (Method D): t_{R} 3.81 min, 100%, MS (ESI) 435.3 (M + H)⁺ |
| 00032 | 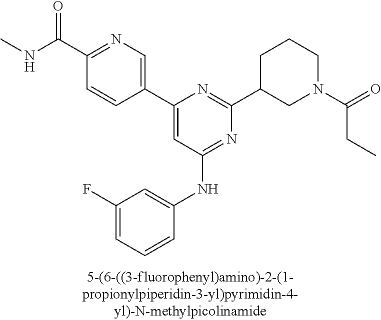 [SMILES: CCC(=O)N1CCCC(c2nc(Nc3cccc(F)c3)cc(-c3ccc(C(=O)NC)nc3)n2)C1] | ¹H-NMR (400 MHz, DMSO-d₆) mixture of rotamers δ 10.07 (d, J = 11.3 Hz, 1H), 9.27-9.17 (m, 1H), 8.97-8.81 (m, 1H), 8.62-8.52 (m, 1H), 8.19 (d, J = 8.2 Hz, 1H), 7.99-7.82 (m, 1H), 7.49-7.32 (m, 2H), 7.25 (d, J = 3.8 Hz, 1H), 6.92-6.79 (m, 1H), 4.83- 4.68 (m, 0.5H), 4.37-4.12 (m, 1H), 4.00-3.82 (m, 0.5H), 3.56-3.38 (m, 0.5H), 3.11-3.01 (m, 0.5H), 3.01- 2.72 (m, 5H), 2.42-2.31 (m, 2H), 2.30-2.18 (m, 1H), 2.00-1.72 (m, 2H), 1.66-1.40 (m, 1H), 1.05-0.94 (m, 3H) ); LCMS (Method D): t_{R} 3.36 min, 100%, MS (ESI) 463.3 (M + H)⁺ |
| 00033 | 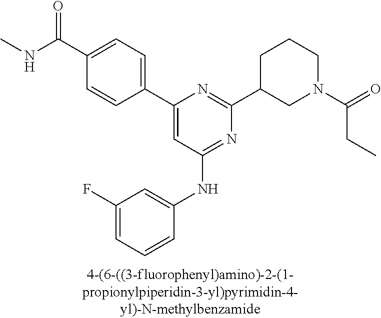 [SMILES: CCC(=O)N1CCCC(c2nc(Nc3cccc(F)c3)cc(-c3ccc(C(=O)NC)cc3)n2)C1] | ¹H-NMR (400 MHz, DMSO-d₆) mixture of rotamers δ 9.96 (d, J = 9.6 Hz, 1H), 8.64-8.53 (m, 1H), 8.18-8.06 (m, 2H), 8.03-7.83 (m, 3H), 7.46-7.33 (m, 2H), 7.22-7.14 (m, 1H), 6.91- 6.78 (m, 1H), 4.86-4.70 (m, 0.5H), 4.34-4.12 (m, 1H), 3.99-3.84 (m, 0.5H), 3.51-3.40 (m, 0.5H), 3.11- 3.00 (m, 0.5H), 3.00-2.71 (m, 5H), 2.44-2.31 (m, 2H), 2.31-2.17 (m, 1H), 1.98-1.71 (m, 2H), 1.65-1.39 (m, 1H), 1.08-0.92 (m, 3H); LCMS (Method D): t_{R} 3.32 min, 100%, MS (ESI) 462.3 (M + H)⁺ |
| 00034 | 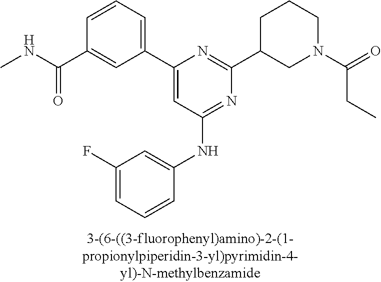 [SMILES: CCC(=O)N1CCCC(c2nc(Nc3cccc(F)c3)cc(-c3cccc(C(=O)NC)c3)n2)C1] | ¹H-NMR (400 MHz, DMSO-d₆) mixture of rotamers δ 9.96 (d, J = 10.5 Hz, 1H), 8.73-8.58 (m, 1H), 8.55-8.44 (m, 1H), 8.27-8.15 (m, 1H), 8.01- 7.85 (m, 2H), 7.69-7.58 (m, 1H), 7.47-7.32 (m, 2H), 7.21 (d, J = 4.0 Hz, 1H), 6.93-6.77 (m, 1H), 4.80- 4.63 (m, 0.5H), 4.36-4.12 (m, 1H), 3.96-3.83 (m, 0.5H), 3.54-3.42 (m, 0.5H), 3.14-3.03 (m, 0.5H), 3.01- 2.89 (m, 1H), 2.89-2.72 (m, 4H), 2.44-2.31 (m, 2H), 2.30-2.16 (m, 1H), 1.98-1.72 (m, 2H), 1.67-1.40 (m, 1H), 1.07-0.89 (m, 3H); LCMS (Method D): t_{R} 3.38 min, 100%, MS (ESI) 462.3 (M + H)⁺ |
| 00035 | 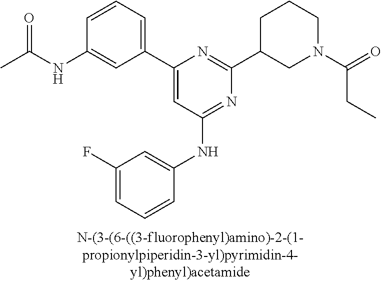 [SMILES: CCC(=O)N1CCCC(c2nc(Nc3cccc(F)c3)cc(-c3cccc(NC(C)=O)c3)n2)C1] | ¹H-NMR (400 MHz, DMSO-d₆) mixture of rotamers δ 10.16 (d, J = 3.8 Hz, 1H), 9.97 (d, J = 10.9 Hz, 1H), 8.46- 8.32 (m, 1H), 8.00-7.84 (m, 1H), 7.78-7.65 (m, 2H), 7.50-7.32 (m, 3H), 7.10 (d, J = 2.6 Hz, 1H), 6.90- 6.78 (m, 1H), 4.88-4.71 (m, 0.5H), 4.36-4.14 (m, 1H), 4.00-3.85 (m, 0.5H), 3.50-3.39 (m, 0.5H), 3.10- 2.98 (m, 0.5H), 2.97-2.69 (m, 2H), 2.45-2.31 (m, 2H), 2.31-2.17 (m, 1H), 2.08 (s, 3H), 1.97-1.73 (m, 2H), 1.66-1.39 (m, 1H), 1.08-0.94 (m, 3H); LCMS (Method D): t_{R} 3.43 min, 100%, MS (ESI) 462.3 (M + H)⁺ |
| 00036 | 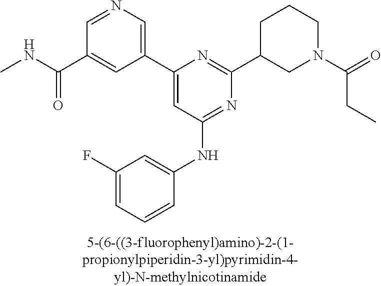 [SMILES: CCC(=O)N1CCCC(c2nc(Nc3cccc(F)c3)cc(-c3cncc(C(=O)NC)c3)n2)C1] | ¹H-NMR (400 MHz, DMSO-d₆) mixture of rotamers δ 10.03 (d, J = 11.5 Hz, 1H), 9.33-9.30 (m, 1H), 9.13-9.04 (m, 1H), 8.91-8.79 (m, 1H), 8.79- 8.71 (m, 1H), 7.98-7.82 (m, 1H), 7.47-7.33 (m, 2H), 7.26 (d, J = 3.2 Hz, 1H), 6.94-6.79 (m, 1H), 4.71 (d, J = 12.8 Hz, 0.5H), 4.34-4.13 (m, 1H), 3.93-3.82 (m, 0.5H), 3.54-3.41 (m, 2H), 3.15-3.02 (m, 0.5H), 3.02- 2.90 (m, 1H), 2.88-2.74 (m, 4.5H), 2.44-2.31 (m, 2.5H), 2.29-2.15 (m, 1H), 1.95-1.73 (m, 2H), 1.65-1.41 (m, 1H), 1.37 (d, J = 6.1 Hz, 0.5H), 1.00 (q, J = 7.5 Hz, 3H); LCMS (Method D): t_{R} 3.19 min, 100%, MS (ESI) 463.3 (M + H)⁺ |
| 00037 | 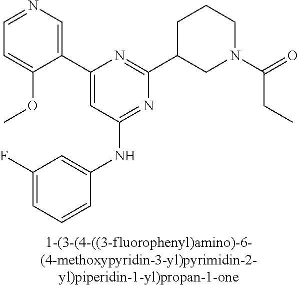 [SMILES: CCC(=O)N1CCCC(c2nc(Nc3cccc(F)c3)cc(-c3cnccc3OC)n2)C1] | ¹H-NMR (400 MHz, DMSO-d₆) mixture of rotamers δ 9.91 (d, J = 9.9 Hz, 1H), 9.02 (d, J = 2.3 Hz, 1H), 8.54 (d, J = 5.8 Hz, 1H), 8.01-7.80 (m, 1H), 7.50- 7.29 (m, 3H), 7.25 (d, J = 5.9 Hz, 1H), 6.94-6.76 (m, 1H), 4.86-4.66 (m, 0.5H), 4.38-4.12 (m, 1H), 3.99 (s, 3H), 3.94-3.83 (m, 0.5H), 3.56- 3.31 (m, 0.5H), 3.12-2.99 (m, 0.5H), 2.98-2.70 (m, 2H), 2.43-2.29 (m, 2H), 2.28-2.16 (m, 1H), 1.98-1.71 (m, 2H), 1.67-1.38 (m, 1H), 1.10- 0.89 (m, 3H); LCMS (Method D): t_{R} 3.31 min, 100%, MS (ESI) 436.3 (M + H)⁺ |
| 00038 | 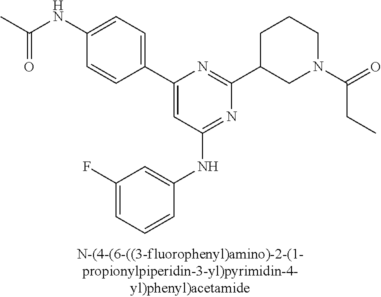 [SMILES: CCC(=O)N1CCCC(c2nc(Nc3cccc(F)c3)cc(-c3ccc(NC(C)=O)cc3)n2)C1] | ¹H-NMR (400 MHz, DMSO-d₆) mixture of rotamers δ 10.17 (s, 1H), 9.85 (d, J = 9.8 Hz, 1H), 8.01 (d, J = 8.3 Hz, 2H), 7.96-7.83 (m, 1H), 7.74 (d, J = 8.5 Hz, 2H), 7.47-7.28 (m, 2H), 7.06 (d, J = 1.9 Hz, 1H), 6.82 (t, J = 8.4 Hz, 1H), 4.88-4.67 (m, 0.5H), 4.35-4.08 (m, 1H), 4.01-3.81 (m, 0.5H), 3.51- 3.40 (m, 0.5H), 3.11-2.98 (m, 0.5H), 2.97-2.69 (m, 2H), 2.44-2.31 (m, 2H), 2.29-2.15 (m, 1H), 2.08 (s, 3H), 1.99-1.70 (m, 2H), 1.65-1.38 (m, 1H), 1.10-0.91 (m, 3H); LCMS (Method D): t_{R} 3.41 min, 100%, MS (ESI) 462.3 (M + H)⁺ |
| 00039 | 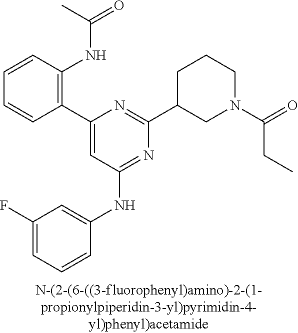 [SMILES: CCC(=O)N1CCCC(c2nc(Nc3cccc(F)c3)cc(-c3ccccc3NC(C)=O)n2)C1] | ¹H-NMR (400 MHz, DMSO-d₆) mixture of rotamers δ 11.14, (s, 0.5 H), 11.02 (s, 0.5H), 10.04 (d, J = 10.4 Hz, 1H), 8.26-8.11 (m, 1H), 7.97-7.80 (m, 1H), 7.66 (d, J = 7.8 Hz, 1H), 7.49- 7.33 (m, 3H), 7.24 (t, J = 7.6 Hz, 1H), 7.00 (s, 1H), 6.90-6.82 (m, 1H), 4.97- 4.75 (m, 0.5H), 4.47-4.32 (m, 0.5H), 4.31-4.18 (m, 0.5H), 4.00- 3.85 (m, 0.5H), 3.44-3.28 (m, 0.5H), 3.10-2.90 (m, 1H), 2.85-2.77 (m, 1H), 2.70-2.60 (m, 0.5H), 2.44-2.31 (m, 2H), 2.30-2.21 (m, 1H), 2.17- 2.04 (m, 3H), 1.98-1.70 (m, 2H), 1.66-1.38 (m, 1H), 1.09-0.91 (m, 3H); LCMS (Method D): t_{R} 3.56 min, 100%, MS (ESI) 462.3 (M + H)⁺ |
| 00040 | 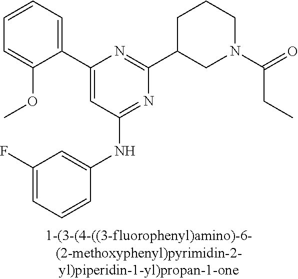 [SMILES: CCC(=O)N1CCCC(c2nc(Nc3cccc(F)c3)cc(-c3ccccc3OC)n2)C1] | ¹H-NMR (400 MHz, DMSO-d₆) mixture of rotamers δ 9.84 (d, J = 9.9 Hz, 1H), 8.08-7.96 (m, 1H), 7.96-7.80 (m, 1H), 7.53-7.28 (m, 4H), 7.23-7.15 (m, 1H), 7.09 (t, J = 7.5 Hz, 1H), 6.89- 6.72 (m, 1H), 4.88-4.68 (m, 0.5H), 4.32-4.11 (m, 1H), 3.99-3.81 (m, 3.5H), 3.55-3.33 (m, 0.5H), 3.10- 2.97 (m, 0.5H), 2.96-2.64 (m, 2H), 2.43-2.31 (m, 2H), 2.29-2.13 (m, 1H), 1.98-1.65 (m, 2H), 1.65-1.36 (m, 1H), 1.09-0.89 (m, 3H); LCMS (Method D): t_{R} 3.79 min, 100%, MS (ESI) 435.3 (M + H)⁺ |
| 00041 | 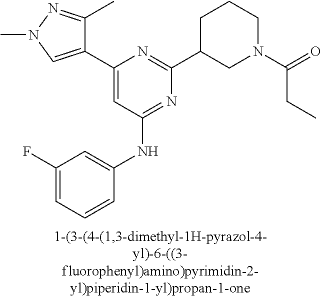 [SMILES: CCC(=O)N1CCCC(c2nc(Nc3cccc(F)c3)cc(-c3cn(C)nc3C)n2)C1] | ¹H-NMR (400 MHz, DMSO-d₆) mixture of rotamers δ 9.72 (d, J = 10.9 Hz, 1H), 8.19 (d, J = 2.7 Hz, 1H), 7.97- 7.78 (m, 1H), 7.42-7.26 (m, 2H), 6.89-6.73 (m, 2H), 4.85-4.71 (m, 0.5H), 4.35-4.14 (m, 1H), 3.96-3.86 (m, 0.5H), 3.82 (s, 3H), 3.44-3.29 (m, 0.5H), 3.07-2.94 (m, 0.5H), 2.89- 2.73 (m, 1H), 2.73-2.63 (m, 1H), 2.48-2.44 (m, 3H), 2.43-2.30 (m, 2H), 2.27-2.14 (m, 1H), 1.93-1.69 (m, 2H), 1.64-1.35 (m, 1H), 1.07- 0.95 (m, 3H); LCMS (Method D): t_{R} 3.36 min, 100%, MS (ESI) 423.3 (M + H)⁺ |
| 00042 | 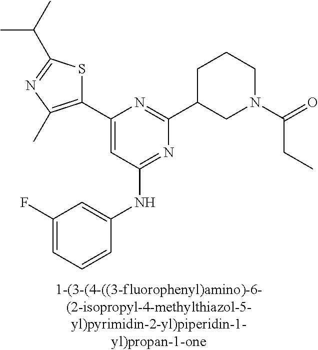 [SMILES: CCC(=O)N1CCCC(c2nc(Nc3cccc(F)c3)cc(-c3sc(C(C)C)nc3C)n2)C1] | ¹H-NMR (400 MHz, DMSO-d₆) mixture of rotamers δ 9.93 (d, J = 10.3 Hz, 1H), 7.99-7.80 (m, 1H), 7.45-7.29 (m, 2H), 6.95 (s, 1H), 6.89-6.77 (m, 1H), 4.83-4.68 (m, 0.5H), 4.31-4.11 (m, 1H), 3.96-3.81 (m, 0.5H), 3.47- 3.19 (m, 1H), 3.08-2.95 (m, 0.5H), 2.93-2.61 (m, 5.5H), 2.42-2.29 (m, 2H), 2.24-2.12 (m, 1H), 1.92-1.67 (m, 2H), 1.63-1.39 (m, 1H), 1.34 (d, J = 6.9 Hz, 6H), 1.06-0.90 (m, 3H); LCMS (Method D): t_{R} 3.93 min, 100%, MS (ESI) 468.3 (M + H)⁺ |
| 00043 | 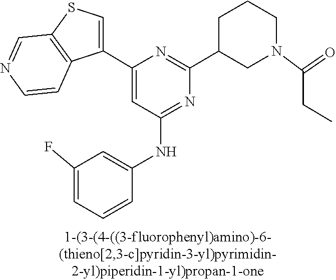 [SMILES: CCC(=O)N1CCCC(c2nc(Nc3cccc(F)c3)cc(-c3csc4cnccc34)n2)C1] | ¹H-NMR (400 MHz, DMSO-d₆) mixture of rotamers δ 9.98 (d, J = 11.2 Hz, 1H), 9.36 (s, 1H), 8.77-8.69 (m, 1H), 8.66-8.50 (m, 2H), 7.99-7.81 (m, 1H), 7.48-7.32 (m, 2H), 7.15 (d, J = 3.7 Hz, 1H), 6.90-6.79 (m, 1H), 4.90- 4.75 (m, 0.5H), 4.41-4.20 (m, 1H), 3.99-3.86 (m, 0.5H), 3.42 (dd, J = 13.4, 10.6 Hz, 0.5H), 3.13-2.64 (m, 2.5H), 2.47-2.19 (m, 3H), 1.99-1.73 (m, 2H), 1.67-1.40 (m, 1H), 1.06- 0.95 (m, 3H); LCMS (Method D): t_{R} 3.57 min, 100%, MS (ESI) 462.2 (M + H)⁺ |
| 00044 | 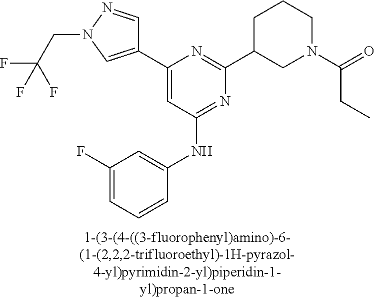 | ¹H-NMR (400 MHz, DMSO-d₆) mixture of rotamers δ 9.80 (d, J = 10.4 Hz, 1H), 8.47 (s, 1H), 8.18-8.06 (m, 1H), 7.94-7.77 (m, 1H), 7.45-7.28 (m, 2H), 6.89-6.74 (m, 2H), 5.24 (q, J = 9.1 Hz, 2H), 4.88-4.59 (m, 0.5H), 4.33-4.09 (m, 1H), 3.99-3.83 (m, 0.5H), 3.42 (dd, J = 13.4, 10.2 Hz, 0.5H), 3.11-2.96 (m, 0.5H), 2.94- 2.64 (m, 2H), 2.43-2.28 (m, 2H), 2.27-2.09 (m, 1H), 1.95-1.67 (m, 2H), 1.65-1.36 (m, 1H), 1.07-0.93 (m, 3H); LCMS (Method D): t_{R} 3.19 min, 100%, MS (ESI) 463.3 (M + H)⁺ |
| 00045 | 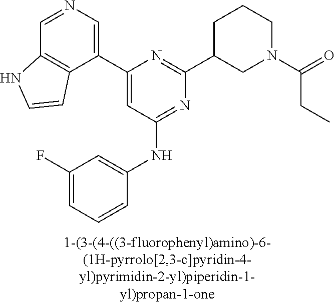 [SMILES: CCC(=O)N1CCCC(c2nc(Nc3cccc(F)c3)cc(-c3cncc4[nH]ccc34)n2)C1] | ¹H-NMR (400 MHz, DMSO-d₆) mixture of rotamers δ 11.91 (s, 1H), 9.91 (d, J = 10.7 Hz, 1H), 8.85 (s, 1H), 8.78- 8.69 (m, 1H), 8.23 (s, 1H), 8.05-7.86 (m, 1H), 7.80 (s, 1H), 7.50-7.33 (m, 2H), 7.29 (d, J = 3.4 Hz, 1H), 7.13- 7.01 (m, 1H), 6.90-6.78 (m, 1H), 4.93-4.78 (m, 0.5H), 4.39-4.20 (m, 1H), 3.98-3.87 (m, 0.5H), 3.54-3.30 (m, 0.5H), 3.15-2.93 (m, 1.5H), 2.92- 2.66 (m, 2H), 2.45-2.23 (m, 3H), 2.04-1.73 (m, 2H), 1.68-1.40 (m, 1H), 1.09-0.94 (m, 3H); LCMS (Method D): t_{R} 3.32 min, 100%, MS (ESI) 445.3 (M + H)⁺ |
| 00046 | 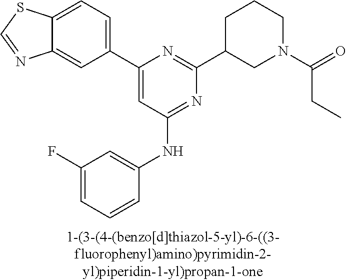 [SMILES: CCC(=O)N1CCCC(c2nc(Nc3cccc(F)c3)cc(-c3ccc4scnc4c3)n2)C1] | ¹H-NMR (400 MHz, DMSO-d₆) mixture of rotamers δ 9.93 (d, J = 10.2 Hz, 1H), 9.50 (s, 1H), 8.74 (s, 1H), 8.34 (d, J = 8.4 Hz, 1H), 8.17 (dd, J = 8.4, 1.7 Hz, 1H), 8.02-7.80 (m, 1H), 7.51- 7.32 (m, 2H), 7.26 (d, J = 2.1 Hz, 1H), 6.90-6.77 (m, 1H), 4.93-4.70 (m, 0.5H), 4.35-4.15 (m, 1H), 3.99-3.82 (m, 0.5H), 3.54-3.44 (m, 0.5H), 3.14- 3.02 (m, 0.5H), 3.02-2.72 (m, 2H), 2.47-2.20 (m, 3H), 2.05-1.72 (m, 2H), 1.68-1.40 (m, 1H), 1.08-0.91 (m, 3H); LCMS (Method D): t_{R} 3.69 min, 100%, MS (ESI) 462.2 (M + H)⁺ |
| 00047 | 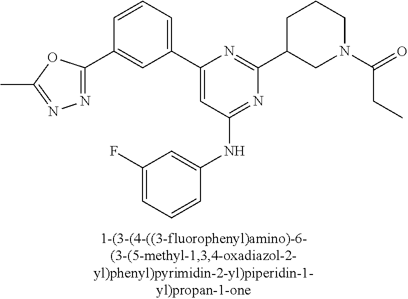 [SMILES: CCC(=O)N1CCCC(c2nc(Nc3cccc(F)c3)cc(-c3cccc(-c4nnc(C)o4)c3)n2)C1] | ¹H-NMR (400 MHz, DMSO-d6) mixture of rotamers δ 10.00 (d, J = 10.8 Hz, 1H), 8.67-8.59 (m, 1H), 8.29 (d, J = 7.9 Hz, 1H), 8.12 (d, J = 7.7 Hz, 1H), 8.00-7.84 (m, 1H), 7.78 (t, J = 7.8 Hz, 1H), 7.48-7.31 (m, 2H), 7.26 (d, J = 3.4 Hz, 1H), 6.93-6.77 (m, 1H), 4.86-4.72 (m, 0.5H), 4.36-4.14 (m, 1H), 3.99-3.87 (m, 0.5H), 3.46 (dd, J = 13.4, 10.3 Hz, 0.5H), 3.13-3.02 (m, 0.5H), 3.02-2.70 (m, 2H), 2.62 (s, 3H), 2.46-2.31 (m, 2H), 2.31-2.20 (m, 1H), 1.99-1.73 (m, 2H), 1.67- 1.42 (m, 1H), 1.06-0.94 (m, 3H); LCMS (Method D): t_{R} 3.60 min, 100%, MS (ESI) 487.3 (M + H)⁺ |
| 00048 | 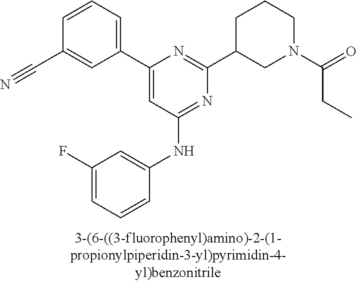 [SMILES: CCC(=O)N1CCCC(c2nc(Nc3cccc(F)c3)cc(-c3cccc(C#N)c3)n2)C1] | ¹H-NMR (400 MHz, DMSO-d6) mixture of rotamers δ 9.98 (d, J = 10.6 Hz, 1H), 8.45 (s, 1H), 8.35 (d, J = 7.9 Hz, 1H), 8.00 (d, J = 7.7 Hz, 1H), 7.95- 7.81 (m, 1H), 7.77 (t, J = 7.8 Hz, 1H), 7.48-7.32 (m, 2H), 7.20 (s, 1H), 6.93- 6.79 (m, 1H), 4.85-4.67 (m, 0.5H), 4.33-4.12 (m, 1H), 3.96-3.83 (m, 0.5H), 3.53-3.40 (m, 0.5H), 3.14- 3.00 (m, 0.5H), 3.00-2.85 (m, 1H), 2.85-2.71 (m, 1H), 2.45-2.31 (m, 2H), 2.29-2.16 (m, 1H), 2.02-1.71 (m, 2H), 1.65-1.40 (m, 1H), 1.05- 0.92 (m, 3H); LCMS (Method D): t_{R} 3.75 min, 100%, MS (ESI) 430.3 (M + H)⁺ |
| 00049 | 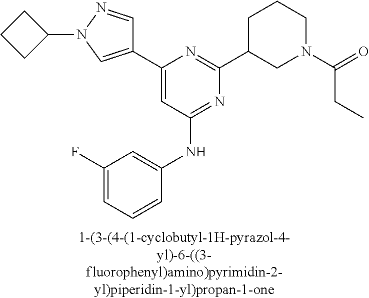 [SMILES: CCC(=O)N1CCCC(c2nc(Nc3cccc(F)c3)cc(-c3cnn(C4CCC4)c3)n2)C1] | ¹H-NMR (400 MHz, DMSO-d6) mixture of rotamers δ 9.76 (d, J = 10.7 Hz, 1H), 8.38 (s, 1H), 8.00 (d, J = 4.0 Hz, 1H), 7.93-7.76 (m, 1H), 7.45-7.29 (m, 2H), 6.87-6.73 (m, 2H), 4.92 (p, J = 8.3 Hz, 1H), 4.78-4.63 (m, 0.5H), 4.33-4.07 (m, 1H), 3.99-3.80 (m, 0.5H), 3.50-3.38 (m, 0.5H), 3.13- 2.96 (m, 0.5H), 2.91-2.62 (m, 2H), 2.59-2.45 (m, 1H), 2.45-2.30 (m, 5H), 2.25-2.10 (m, 1H), 1.94-1.67 (m, 4H), 1.63-1.36 (m, 1H), 1.07- 0.94 (m, 3H); LCMS (Method D): t_{R} 3.58 min, 100%, MS (ESI) 449.3 (M + H)⁺ |
| 00050 | 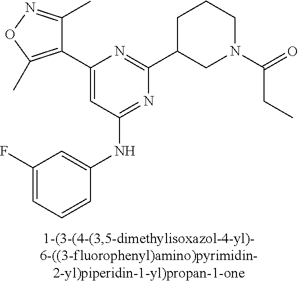 [SMILES: CCC(=O)N1CCCC(c2nc(Nc3cccc(F)c3)cc(-c3c(C)noc3C)n2)C1] | ¹H-NMR (400 MHz, DMSO-d6) mixture of rotamers δ 9.89 (d, J = 12.1 Hz, 1H), 8.00-7.77 (m, 1H), 7.45-7.28 (m, 2H), 6.93-6.74 (m, 2H), 4.90- 4.71 (m, 0.5H), 4.42-4.29 (m, 0.5H), 4.29-4.16 (m, 0.5H), 3.99-3.80 (m, 0.5H), 3.08-2.96 (m, 0.5H), 2.95- 2.59 (m, 5.5H), 2.48-2.43 (m, 3H), 2.43-2.31 (m, 2H), 2.30-2.16 (m, 1H), 1.91-1.66 (m, 2H), 1.66-1.35 (m, 1H), 1.00 (t, J = 7.4 Hz, 3H); LCMS (Method D): t_{R} 3.60 min, 100%, MS (ESI) 424.3 (M + H)⁺ |
| 00051 | 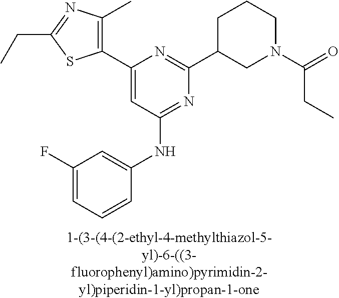 [SMILES: CCC(=O)N1CCCC(c2nc(Nc3cccc(F)c3)cc(-c3sc(CC)nc3C)n2)C1] | ¹H-NMR (400 MHz, DMSO-d6) mixture of rotamers δ 9.92 (d, J = 10.7 Hz, 1H), 8.01-7.72 (m, 1H), 7.45-7.21 (m, 2H), 7.02-6.70 (m, 2H), 4.82- 4.65 (m, 0.5H), 4.33-4.07 (m, 1H), 3.96-3.79 (m, 0.5H), 3.07-2.59 (m, 8H), 2.43-2.26 (m, 2H), 2.25-2.05 (m, 1H), 1.97-1.66 (m, 2H), 1.63- 1.38 (m, 1H), 1.38-1.19 (m, 3H), 1.07-0.87 (m, 3H); LCMS (Method D): t_{R} 3.77 min, 100%, MS (ESI) 454.4 (M + H)⁺ |
| 00052 | 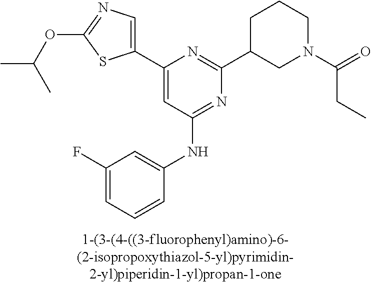 | ¹H-NMR (400 MHz, DMSO-d6) mixture of rotamers δ 9.90 (d, J = 11.1 Hz, 1H), 7.92 (d, J = 3.9 Hz, 1H), 7.83 (m, 1H), 7.45-7.29 (m, 2H), 6.91 (s, 1H), 6.88-6.77 (m, 1H), 5.18 (hept, J = 6.2 Hz, 1H), 4.78-4.62 (m, 0.5H), 4.29-4.05 (m, 1H), 3.94-3.82 (m, 0.5H), 3.45-3.34 (m, 0.5H), 3.08- 2.97 (m, 0.5H), 2.90-2.62 (m, 2H), 2.42-2.30 (m, 2H), 2.22-2.08 (m, 1H), 1.92-1.67 (m, 2H), 1.61-1.31 (m, 7H), 1.06-0.93 (m, 3H); LCMS (Method D): t_{R} 3.97 min, 100%, MS (ESI) 4670.3 (M + H)⁺ |
| 00053 | 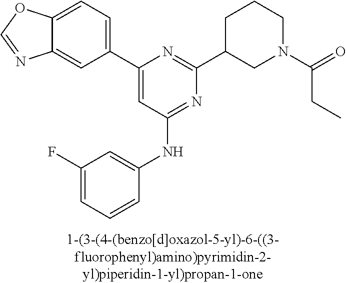 [SMILES: CCC(=O)N1CCCC(c2nc(Nc3cccc(F)c3)cc(-c3ccc4ocnc4c3)n2)C1] | ¹H-NMR (400 MHz, DMSO-d₆) mixture of rotamers δ 9.92 (d, J = 11.0 Hz, 1H), 8.85 (s, 1H), 8.46 (s, 1H), 8.16 (d, J = 8.6 Hz, 1H), 8.00-7.83 (m, 2H), 7.49-7.29 (m, 2H), 7.21 (s, 1H), 6.92- 6.76 (m, 1H), ), 4.89-4.71 (m, 0.5H), 4.36-4.13 (m, 1H), 4.00-3.85 (m, 0.5H), 3.55-3.40 (m, 0.5H), 3.14- 3.00 (m, 0.5H), 3.00-2.71 (m, 2H), 2.45-2.31 (m, 2H), 2.31-2.20 (m, 1H), 2.01-1.71 (m, 2H), 1.68-1.39 (m, 1H), 1.09-0.92 (m, 3H); LCMS (Method D): t_{R} 3.63 min, 100%, MS (ESI) 446.3 (M + H)⁺ |
| 00054 | 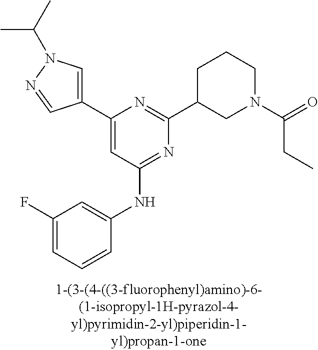 [SMILES: CCC(=O)N1CCCC(c2nc(Nc3cccc(F)c3)cc(-c3cnn(C(C)C)c3)n2)C1] | ¹H-NMR (400 MHz, DMSO-d₆) mixture of rotamers δ 9.85-9.60 (m, 1H), 8.33 (s, 1H), 7.97 (d, J = 3.6 Hz, 1H), 7.92-7.77 (m, 1H), 7.45-7.27 (m, 2H), 6.80 (s, 2H), 4.77-4.66 (m, 0.5H), 4.66-4.52 (m, 1H), 4.31-4.19 (m, 0.5H), 4.18-4.08 (m, 0.5H), 3.96- 3.82 (m, 0.5H), 3.51-3.39 (m, 0.5H), 3.10-2.96 (m, 0.5H), 2.92- 2.62 (m, 2H), 2.44-2.31 (m, 2H), 2.24-2.12 (m, 1H), 1.94-1.70 (m, 2H), 1.66-1.35 (m, 7H), 1.07-0.93 (m, 3H) ); LCMS (Method D): t_{R} 3.51 min, 100%, MS (ESI) 437.3 (M + H)⁺ |
| 00055 | 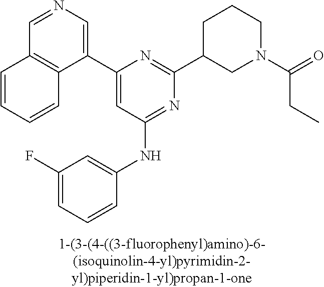 [SMILES: CCC(=O)N1CCCC(c2nc(Nc3cccc(F)c3)cc(-c3cncc4ccccc34)n2)C1] | ¹H-NMR (400 MHz, DMSO-d₆) mixture of rotamers δ 10.01 (d, J = 9.9 Hz, 1H), 9.43 (s, 1H), 8.67 (s, 1H), 8.33 (t, J = 9.2 Hz, 1H), 8.25 (d, J = 8.1 Hz, 1H), 8.02-7.82 (m, 2H), 7.77 (t, J = 7.5 Hz, 1H), 7.49-7.33 (m, 2H), 7.00 (d, J = 5.8 Hz, 1H), 6.90-6.81 (m, 1H), 4.90-4.73 (m, 0.5H), 4.33-4.15 (m, 1H), 3.95-3.81 (m, 0.5H), 3.50- 3.33 (m, 0.5H), 3.12-2.93 (m, 1H), 2.92-2.69 (m, 1.5H), 2.42-2.30 (m, 2H), 2.30-2.20 (m, 1H), 1.99-1.70 (m, 2H), 1.64-1.38 (m, 1H), 1.07- 0.91 (m, 3H); LCMS (Method D): t_{R} 3.54 min, 100%, MS (ESI) 456.3 (M + H)⁺ |
| 00056 | 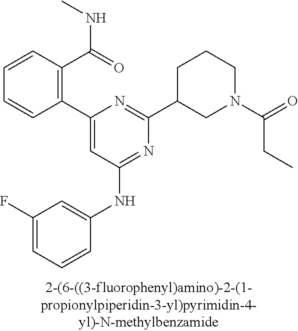 [SMILES: CCC(=O)N1CCCC(c2nc(Nc3cccc(F)c3)cc(-c3ccccc3C(=O)NC)n2)C1] | ¹H-NMR (400 MHz, DMSO-d₆) mixture of rotamers δ 9.90 (d, J = 11.3 Hz, 1H), 8.29-8.12 (m, 1H), 7.97-7.79 (m, 1H), 7.67 (d, J = 7.4 Hz, 1H), 7.61- 7.30 (m, 5H), 6.91-6.77 (m, 2H), 4.85-4.68 (m, 0.5H), 4.41-4.29 (m, 0.5H), 4.23-4.11 (m, 0.5H), 3.99- 3.85 (m, 0.5H), 3.48-3.22 (m, 0.5H), 3.05-2.93 (m, 0.5H), 2.90-2.60 (m, 5H), 2.43-2.31 (m, 2H), 2.26-2.13 (m, 1H), 1.91-1.65 (m, 2H), 1.64- 1.35 (m, 1H), 1.09-0.93 (m, 3H); LCMS (Method D): t_{R} 3.21 min, 100%, MS (ESI) 462.3 (M + H)⁺ |
| 00057 | 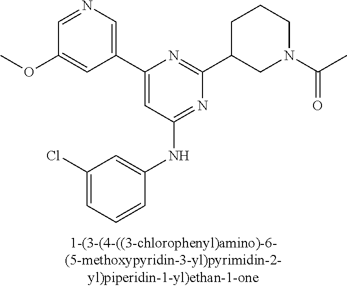 [SMILES: COc1cncc(-c2cc(Nc3cccc(Cl)c3)nc(C3CCCN(C(C)=O)C3)n2)c1] | ¹H-NMR (400 MHz, DMSO-d6)) mixture of rotamers δ 9.97 (d, J = 12.0 Hz, 1H), 8.87-8.71 (m, 1H), 8.51- 8.38 (m, 1H), 8.22-8.02 (m, 1H), 7.95-7.87 (m, 1H), 7.69-7.50 (m, 1H), 7.47-7.33 (m, 1H), 7.24-7.13 (m, 1H), 7.11-7.02 (m, 1H), 4.83- 4.60 (m, 0.5H), 4.24-4.07 (m, 1H), 3.93 (s, 3H), 3.89-3.77 (m, 0.5H), 3.58-3.46 (m, 0.5H), 3.17-3.04 (m, 0.5H), 3.02-2.88 (m, 0.5H), 2.86- 2.73 (m, 1H), 2.29-2.14 (m, 1H), 2.05 (d, J = 4.4 Hz, 3H), 1.97-1.70 (m, 2H), 1.67-1.40 (m, 1H); LCMS (Method D): t_{R} 3.29 min, 100%, MS (ESI) 438.1 (M + H)⁺. |
| 00058 | 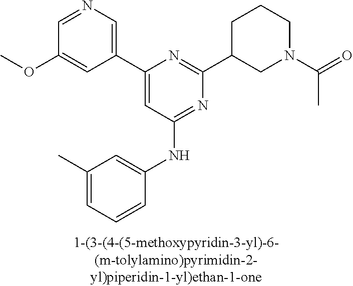 [SMILES: COc1cncc(-c2cc(Nc3cccc(C)c3)nc(C3CCCN(C(C)=O)C3)n2)c1] | ¹H-NMR (400 MHz, DMSO-d6) a mixture of rotamer δ 9.68 (d, J = 8.0 Hz, 1H), 8.78 (dd, J = 6.3, 1.7 Hz, 1H), 8.42 (t, J = 2.7 Hz, 1H), 7.90 (t, J = 2.2 Hz, 1H), 7.63 (d, J = 6.2 Hz, 1H), 7.53 (d, J = 8.3 Hz, 1H), 7.23 (td, J = 7.8, 2.2 Hz, 1H), 7.14 (d, J = 4.7 Hz, 1H), 6.86 (d, J = 7.5 Hz, 1H), 4.74 (d, J = 12.5 Hz, 0.5H), 4.22-4.06 (m, 1H), 3.93 (s, 3H), 3.84 (d, J = 13.6 Hz, 0.5H), 3.54 (dd, J = 13.3, 9.8 Hz, 0.5H), 3.08 (t, J = 12.3 Hz, 0.5H), 2.99- 2.69 (m, 2H), 2.32 (d, J = 2.4 Hz, 3H), 2.28-2.16 (m, 1H), 2.04 (s, 3H), 1.96-1.71 (m, 2H), 1.65-1.40 (m, 1H); LCMS (Method D): t_{R} 3.34 min, 100%, MS (ESI) 418.2 (M + H)⁺ |
| 00059 | 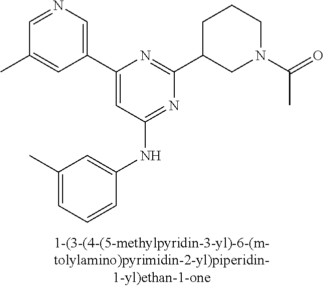 [SMILES: CC(=O)N1CCCC(c2nc(Nc3cccc(C)c3)cc(-c3cncc(C)c3)n2)C1] | ¹H-NMR (400 MHz, DMSO-d6) a mixture of rotamers δ 9.67 (d, J = 8.1 Hz, 1H), 9.02-8.93 (m, 1H), 8.54 (s, 1H), 8.18 (s, 1H), 7.62 (d, J = 5.0 Hz, 1H), 7.53 (d, J = 8.2 Hz, 1H), 7.23 (t, J = 7.9, 2.3 Hz, 1H), 7.13 (d, J = 4.0 Hz, 1H), 6.86 (d, J = 7.4 Hz, 1H), 4.77 (d, J = 12.5 Hz, 0.5H), 4.21 (d, J = 12.9 Hz, 0.5H), 4.14-4.07 (m, 0.5H), 3.86 (d, J = 13.6 Hz, 0.5H), 3.52 (dd, J = 13.4, 10.0.5 Hz, 1H), 3.07 (t, J = 12.6 Hz, 0.5H), 2.98-2.70 (m, 2H), 2.41 (s, 3H), 2.32 (d, J = 2.2 Hz, 3H), 2.23 (d, J = 12.4 Hz, 1H), 2.04 (d, J = 2.1 Hz, 3H), 1.95-1.71 (m, 2H), 1.65- 1.39 (m, 1H); LCMS (Method B): t_{R} 3.34 min, 100%, MS (ESI) 402.2 (M + H)⁺ |
| 00060 | 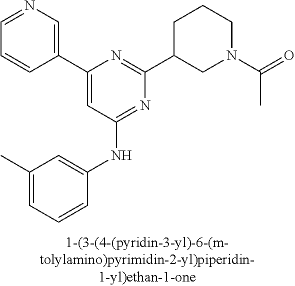 [SMILES: CC(=O)N1CCCC(c2nc(Nc3cccc(C)c3)cc(-c3cccnc3)n2)C1] | ¹H-NMR (400 MHz, DMSO-d6) a mixture of retainers δ 9.72 (d, J = 9.9 Hz, 1H), 9.19 (d, J = 5.3 Hz, 1H), 8.75- 8.64 (m, 1H), 8.37 (d, J = 7.9 Hz, 1H), 7.66-7.49 (m, 3H), 7.29-7.19 (m, 1H), 7.15 (d, J = 5.1 Hz, 1H), 6.86 (d, J = 7.4 Hz, 1H), 4.78 (d, J = 12.4 Hz, 0.5H), 4.21 (d, J = 12.7 Hz, 0.5H), 3.85 (d, J = 13.5 Hz, 0.5H), 3.52 (dd, J = 13.4, 10.1 Hz, 0.5H), 3.07 (t, J = 12.5 Hz, 0.5H), 2.98-2.70 (m, 2H), 2.32 (s, 3H), 2.22 (s, 1H), 2.04 (s, 3H), 1.96-1.71 (m, 2H), 1.66-1.39 (m, 1H); LCMS (Method B): t_{R} 3.26 min, 100%, MS (ESI) 388.2 (M + H)⁺ |
| 00061 | 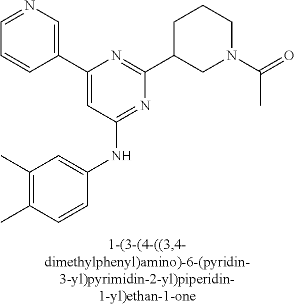 [SMILES: CC(=O)N1CCCC(c2nc(Nc3ccc(C)c(C)c3)cc(-c3cccnc3)n2)C1] | ¹H-NMR (400 MHz, DMSO-d6) mixture or rotamers δ 9.60 (d, J = 8.6 Hz, 1H), 9.23-9.14 (m, 1H), 8.69 (d, J = 4.7 Hz, 1H), 8.40-8.31 (m, 1H), 7.55 (d, J = 7.7 Hz, 2H), 7.43 (s, 1H), 7.14-7.06 (m, 2H), 4.77 (d, J = 12.6 5 Hz, 0.5H), 4.20 (d, J = 12.8 Hz, 0.5H), 4.11 (d, J = 13.4 Hz, 0.5H), 3.85 (d, J = 13.8 Hz, 0.5H), 3.51 (dd, J = 13.4, 10.1 Hz, 0.5H), 3.11-3.01 (m, 0.5H), 2.99-2.64 (m, 2H), 2.27-2.14 (m, 7H), 2.04 (d, J = 3.3 Hz, 3H), 1.95- 1.70 (m, 2H), 1.65-1.38 (m, 1H); LCMS (Method B): t_{R} 3.35 min, 100%, MS (ESI) 402.2 (M + H)⁺ |
| 00062 | 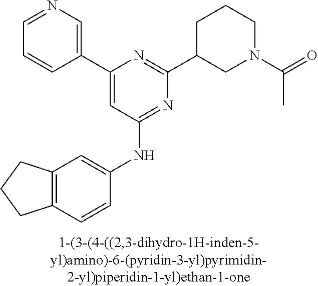 [SMILES: CC(=O)N1CCCC(c2nc(Nc3ccc4c(c3)CCC4)cc(-c3cccnc3)n2)C1] | ¹H-NMR (400 MHz, DMSO-d6) mixture of rotamers δ 9.64 (d, J = 11.4 Hz, 1H), 9.18 (d, J = 5.1 Hz, 1H), 8.69 (d, J = 4.7 Hz, 1H), 8.36 (d, J = 8.0 Hz, 1H), 7.64 (d, J = 12.2 Hz, 1H), 7.56 (t, J = 6.5 Hz, 1H), 7.42 (d, J = 8.1 Hz, 1H), 7.19 (d, J = 8.1 Hz, 1H), 7.10 (d, J = 5.6 Hz, 1H), 4.75 (d, J = 13.7 Hz, 0.5H), 4.21 (d, J = 13.1 Hz, 0.5H), 4.12 (d, J = 14.6 Hz, 0.5H), 3.85 (d, J = 13.7 Hz, 0.5H), 3.49 (dd, J = 13.5, 10.1 Hz, 0.5H), 3.06 (t, J = 12.6 Hz, 0.5H), 2.96-2.65 (m, 6H), 2.25-2.15 (m, 1H), 2.03 (d, J = 5.0 Hz, 5H), 1.94- 1.71 (m, 2H), 1.51 (dd, J = 46.6, 12.9 Hz, 1H); LCMS (Method B): t_{R} 3.48 min, 100%, MS (ESI) 414.2 (M + H)⁺ |
| 00063 | 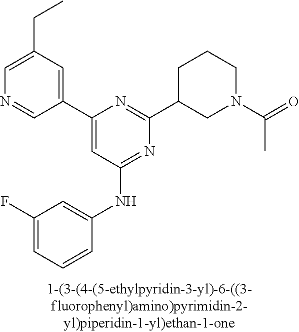 [SMILES: CCc1cncc(-c2cc(Nc3cccc(F)c3)nc(C3CCCN(C(C)=O)C3)n2)c1] | ¹H-NMR (400 MHz, DMSO-d6) mixture of rotamers δ 9.96 (d, J = 9.8 Hz, 1H), 9.02 (d, J = 6.0 Hz, 1H), 8.58 (s, 1H), 8.21 (s, 1H), 7.89 (t, J = 12.4 Hz, 1H), 7.46-7.32 (m, 2H), 7.17 (d, J = 4.3 Hz, 1H), 6.84 (t, J = 8.5 Hz, 1H), 4.73 (d, J = 12.4 Hz, 0.5H), 4.31-4.04 (m, 1H), 3.85 (d, J = 13.5 Hz, 0.5H), 3.62- 3.45 (m, 0.5H), 3.09 (t, J = 12.6 Hz, 0.5H), 3.02-2.93 (m, 0.5H), 2.93- 2.85 (m, 0.5H), 2.84-2.69 (m, 3H), 2.28-2.17 (m, 1H), 2.05 (s, 3H), 1.92- 1.73 (m, 2H), 1.66-1.44 (m, 1H), 1.26 (t, J = 7.5 Hz, 3H); LCMS (Method D): t_{R} 3.45 min, 97%, MS (ESI) 420.2 (M + H)⁺ |
| 00064 | 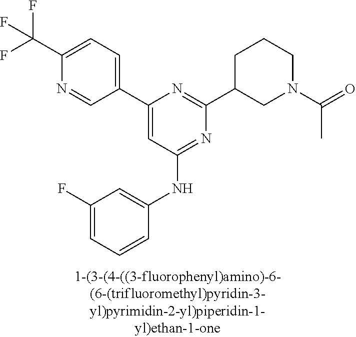 [SMILES: CC(=O)N1CCCC(c2nc(Nc3cccc(F)c3)cc(-c3ccc(C(F)(F)F)nc3)n2)C1] | ¹H-NMR (400 MHz, DMSO-d6) mixture of rotamers δ 10.09 (d, J = 10.3 Hz, 1H), 9.36 (d, J = 7.3 Hz, 1H), 8.67 (d, J = 8.2 Hz, 1H), 8.09 (d, J = 8.3 Hz, 1H), 7.88 (t, J = 12.2 Hz, 1H), 7.47- 7.36 (m, 2H), 7.27 (d, J = 4.6 Hz, 1H), 6.86 (t, J = 8.5 Hz, 1H), 4.74 (d, J = 12.4 Hz, 0.5H), 4.29-4.11 (m, 1H), 3.85 (d, J = 13.5 Hz, 0.5H), 3.58- 3.45 (m, 0.5H), 3.09 (t, J = 12.6 Hz, 0.5H), 3.03-2.95 (m, 0.5H), 2.94- 2.72 (m, 1.5H), 2.28-2.19 (m, 1H), 2.04 (s, 3H), 1.91-1.75 (m, 2H), 1.66- 1.44 (m, 1H); LCMS (Method D): t_{R} 3.69 min, 99%, MS (ESI) 460.1 (M + H)⁺ |
| 00065 | 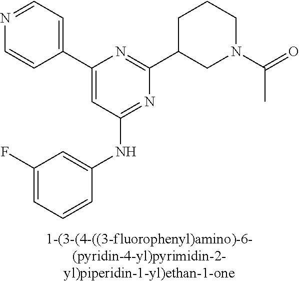 [SMILES: CC(=O)N1CCCC(c2nc(Nc3cccc(F)c3)cc(-c3ccncc3)n2)C1] | ¹H-NMR (400 MHz, DMSO-d6) mixture of rotamers δ 10.04 (d, J = 10.2 Hz, 1H), 8.76 (d, J = 4.8 Hz, 2H), 8.02- 7.94 (m, 2H), 7.93-7.83 (m, 1H), 7.48-7.32 (m, 2H), 7.22 (d, J = 4.5 Hz, 1H), 6.91-6.81 (m, 1H), 4.75 (d, J = 12.2 Hz, 0.5H), 4.29-4.10 (m, 1H), 3.86 (d, J = 13.5 Hz, 0.5H), 3.58- 3.45 (m, 0.5), 3.09 (t, J = 12.5 Hz, 0.5H), 2.98 (m, 0.5H), 2.92-2.72 (m, 1.5H), 2.29-2.19 (m, 1H), 2.04 (s, 3H), 1.91-1.72 (m, 2H), 1.68-1.39 (m, 1H).; LCMS (Method D): t_{R} 3.20 min, 98%, MS (ESI) 392.2 (M + H)⁺ |
| 00066 | 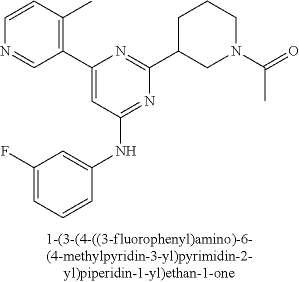 [SMILES: CC(=O)N1CCCC(c2nc(Nc3cccc(F)c3)cc(-c3cnccc3C)n2)C1] | ¹H-NMR (400 MHz, DMSO-d6) mixture of rotamers δ 9.94 (d, J = 9.6 Hz, 1H), 8.60 (d, J = 3.2 Hz, 1H), 8.51 (d, J = 5.1 Hz, 1H), 7.89 (t, J = 12.4 Hz, 1H), 7.45-7.33 (m, 3H), 6.89-6.80 (m, 2H), 4.85-4.61 (m, 0.5H), 4.35-4.03 (m, 1H), 3.94-3.79 (m, 0.5H), 3.50- 3.37 (m, 0.5H), 3.11-3.00 (m, 0.5H), 2.99-2.89 (m, 0.5H), 2.85-2.65 (m, 1.5H), 2.44 (d, J = 2.6 Hz, 3H), 2.27- 2.16 (m, 1H), 2.03 (d, J = 2.3 Hz, 3H), 1.87-1.70 (m, 2H), 1.64-1.38 (m, 1H); LCMS (Method D): t_{R} 3.16 min, 99%, MS (ESI) 406.2 (M + H)⁺ |
| 00067 | 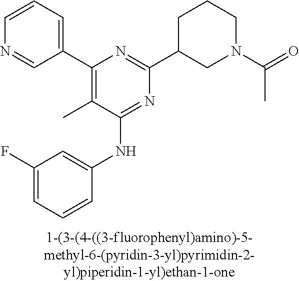 [SMILES: CC(=O)N1CCCC(c2nc(Nc3cccc(F)c3)c(C)c(-c3cccnc3)n2)C1] | ¹H-NMR (400 MHz, DMSO-d6) mixture of rotamers δ 8.82-8.71 (m, 2H), 8.67 (d, J = 4.8 Hz, 1H), 7.99 (dt, J = 7.9, 2.0 Hz, 1H), 7.82 (ddt, J = 11.8, 6.7, 2.3 Hz, 1H), 7.61 (ddd, J = 7.9, 5.6, 1.9 Hz, 1H), 7.54 (ddd, J = 7.4, 4.9, 1.7 Hz, 1H), 7.42-7.33 (m, 1H), 6.92-6.83 (m, 1H), 4.71-4.64 (m, 0.5H), 4.16 (dt, J = 13.0, 4.2 Hz, 0.5H), 4.04 (dd, J = 13.3, 3.9 Hz, 0.5H), 3.82 (d, J = 13.5 Hz, 0.5H), 3.42 (dd, J = 13.4, 10.1 Hz, 0.5H), 3.07-2.98 (m, 0.5H), 2.94-2.83 (m, 0.5H), 2.83-2.64 (m, 1.5H), 2.23 (d, J = 2.6 Hz, 3H), 2.14 (dd, J = 13.6, 3.9 Hz, 1H), 2.00 (d, J = 12.7 Hz, 3H), 1.89-1.64 (m, 2H), 1.61-1.34 (m, 1H); LCMS (Method D): t_{R} 3.12 min, 100%, MS (ESI) 406.1 (M + H)⁺ |
| 00068 | 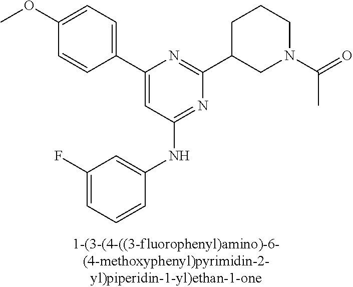 [SMILES: COc1ccc(-c2cc(Nc3cccc(F)c3)nc(C3CCCN(C(C)=O)C3)n2)cc1] | ¹H-NMR (400 MHz, DMSO-d6) mixture of rotamers δ 9.83 (d, J = 10.7 Hz, 1H), 8.09-7.97 (m, 2H), 7.89 (t, J = 12.8 Hz, 1H), 7.45-7.28 (m, 2H), 7.14-7.02 (m, 3H), 6.87-6.75 (m, 1H), 4.83-4.61 (m, 0.5H), 4.30-4.08 (m, 1H), 3.84 (s, 3.5H), 3.58-3.45 (m, 0.5H), 3.07 (t, J = 12.2 Hz, 0.5H), 3.00-2.89 (m, 0.5H), 2.88-2.70 (m, 1.5H), 2.27-2.18 (m, 1H), 2.04 (s, 3H), 1.94-1.74 (m, 2H), 1.65-1.39 (m, 1H); LCMS (Method D): t_{R} 3.70 min, 99%, MS (ESI) 421.2 (M + H)⁺ |
| 00069 | 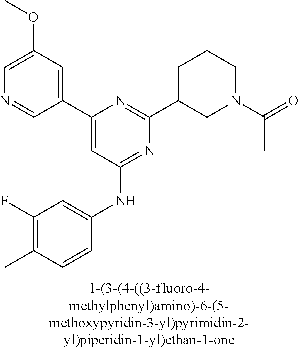 [SMILES: COc1cncc(-c2cc(Nc3ccc(C)c(F)c3)nc(C3CCCN(C(C)=O)C3)n2)c1] | ¹H-NMR (400 MHz, DMSO-d6) mixture of rotamers δ 9.86 (d, J = 11.5 Hz, 1H), 8.89-8.70 (m, 1H), 8.43 (t, J = 2.6 Hz, 1H), 7.90 (t, J = 2.4 Hz, 1H), 7.86-7.70 (m, 1H), 7.37-7.29 (m, 1H), 7.23 (t, J = 8.6 Hz, 1H), 7.14 (d, J = 5.6 Hz, 1H), 4.79-4.62 (m, 0.5H), 4.25-4.09 (m, 1H), 3.93 (s, 3H), 3.89- 3.79 (m, 0.5H), 3.57-3.46 (m, 0.5H), 3.09 (t, J = 12.7 Hz, 0.5H), 3.01- 2.86 (m, 1H), 2.86-2.72 (m, 1H), 2.26-2.17 (m, 4H), 2.04 (s, 3H), 1.95- 1.73 (m, 2H), 1.67-1.39 (m, 1H); LCMS (Method D): t_{R} 3.47 min, 100%, MS (ESI) 436.2 (M + H)⁺ |
| 00070 | 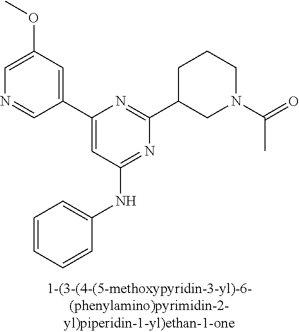 [SMILES: COc1cncc(-c2cc(Nc3ccccc3)nc(C3CCCN(C(C)=O)C3)n2)c1] | ¹H-NMR (400 MHz, DMSO-d6) mixture of rotamers δ 9.74 (d, J = 7.8 Hz, 1H), 8.89-8.73 (m, 1H), 8.42 (t, J = 2.6 Hz, 1H), 7.95-7.84 (m, 1H), 7.76 (d, J = 8.0 Hz, 2H), 7.43-7.30 (m, 2H), 7.16 (d, J = 3.9 Hz, 1H), 7.04 (t, J = 7.3 Hz, 1H), 4.75-4.64 (m, 0.5H), 4.25-4.06 (m, 1H), 3.93 (s, 3H), 3.90- 3.79 (m, 0.5H), 3.58-3.49 (m, 0.5H), 3.19-3.03 (m, 0.5H), 3.00- 2.68 (m, 2H), 2.29-2.15 (m, 1H), 2.04 (d, J = 2.1 Hz, 3H), 1.94-1.71 (m, 2H), 1.65-1.39 (m, 1H).; LCMS (Method D): t_{R} 3.23 min, 100%, MS (ESI) 404.2 (M + H)⁺ |
| 00071 | 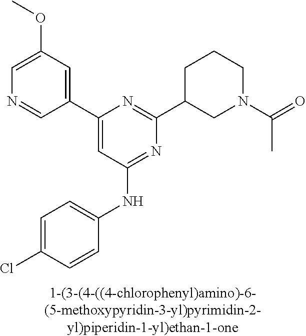 [SMILES: COc1cncc(-c2cc(Nc3ccc(Cl)cc3)nc(C3CCCN(C(C)=O)C3)n2)c1] | ¹H-NMR (400 MHz, DMSO-d6) mixture of rotamers δ 9.88 (d, J = 8.4 Hz, 1H), 8.80 (dd, J = 5.7, 1.7 Hz, 1H), 8.43 (t, J = 2.6 Hz, 1H), 7.91 (t, J = 2.4 Hz, 1H), 7.80 (dd, J = 9.0, 2.9 Hz, 2H), 7.40 (dd, J = 8.9, 3.4 Hz, 2H), 7.16 (d, J = 4.6 Hz, 1H), 4.74-4.59 (m, 0.5H), 4.19 (d, J = 13.1 Hz, 0.5H), 4.09 (d, J = 12.5 Hz, 0.5H), 3.93 (s, 3H), 3.84 (d, J = 13.5 Hz, 0.5H), 3.60-3.47 (m, 0.5H), 3.11 (t, J = 12.3 Hz, 0.5H), 3.01- 2.88 (m, 1H), 2.87-2.72 (m, 1H), 2.20 (d, J = 13.4 Hz, 1H), 2.04 (s, 3H), 1.94-1.70 (m, 2H), 1.65-1.52 (m, 0.5H), 1.52-1.39 (m, 0.5H); LCMS (Method D): t_{R} 3.36 min, 99%, MS (ESI) 438.1 (M + H)⁺ |
| 00072 | 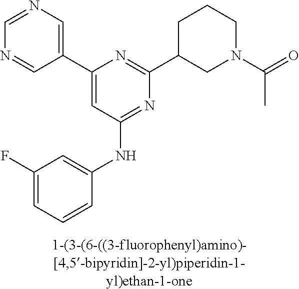 [SMILES: CC(=O)N1CCCC(c2nc(Nc3cccc(F)c3)cc(-c3cncnc3)n2)C1] | ¹H-NMR (400 MHz, DMSO-d6) mixture of rotamers δ 10.08 (d, J = 11.2 Hz, 1H), 9.38 (d, J = 7.1 Hz, 2H), 9.33 (d, J = 2.3 Hz, 1H), 7.93-7.80 (m, 1H), 7.49-7.33 (m, 2H), 7.23 (d, J = 5.7 Hz, 1H), 6.86 (t, J = 8.5 Hz, 1H), 4.72 (d, J = 13.3 Hz, 0.5H), 4.23 (d, J = 12.9 Hz, 0.5H), 4.19-4.11 (m, 0.5H), 3.90-3.80 (m, 0.5H), 3.51 (dd, J = 13.4, 10.2 Hz, 0.5H), 3.15-3.05 (m, 0.5H), 3.02-2.72 (m, 2H), 2.30-2.16 (m, 1H), 2.04 (s, 3H), 1.96-1.71 (m, 2H), 1.67-1.40 (m, 1H); LCMS (Method B): t_{R} 3.09 min, 100%, MS (ESI) 393.1 (M + H)⁺ |
| 00073 | 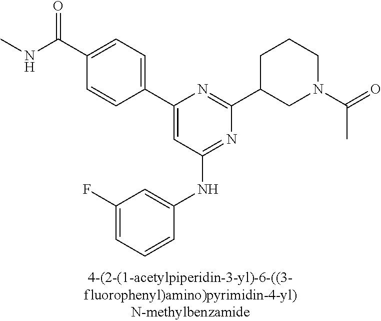 [SMILES: CNC(=O)c1ccc(-c2cc(Nc3cccc(F)c3)nc(C3CCCN(C(C)=O)C3)n2)cc1] | ¹H-NMR (400 MHz, DMSO-d6) mixture of rotamers δ 9.94 (d, J = 10.0 Hz, 1H), 8.61-8.50 (m, 1H), 8.18- 8.09 (m, 2H), 7.98 (d, J = 8.2 Hz, 2H), 7.94-7.84 (m, 1H), 7.45-7.32 (m, 2H), 7.22-7.15 (m, 1H), 6.89-6.80 (m, 1H), 4.81-4.71 (m, 0.5H), 4.28- 4.21 (m, 0.5H), 4.20-4.10 (m, 0.5H), 3.93-3.82 (m, 0.5H), 3.57-3.47 (m, 0.5H), 3.14-3.03 (m, 0.5H), 3.03- 2.92 (m, 0.5H), 2.92-2.71 (m, 4.5H), 2.29-2.18 (m, 1H), 2.05 (s, 3H), 1.97- 1.72 (m, 2H), 1.67-1.40 (m, 1H); LCMS (Method B): t_{R} 2.95 min, 99%, MS (ESI) 448.2 (M + H)⁺ |
| 00074 | 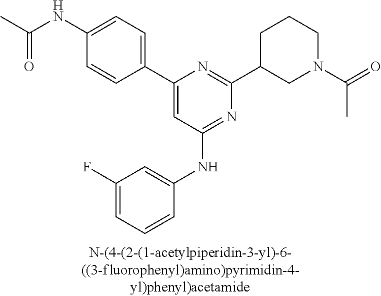 [SMILES: CC(=O)Nc1ccc(-c2cc(Nc3cccc(F)c3)nc(C3CCCN(C(C)=O)C3)n2)cc1] | ¹H-NMR (400 MHz, DMSO-d6) mixture of rotamers δ 10.17 (s, 1H), 9.85 (d, J = 9.8 Hz, 1H), 8.06-7.97 (m, 2H), 7.95-7.84 (m, 1H), 7.74 (d, J = 8.6 Hz, 2H), 7.45-7.30 (m, 2H), 7.10-7.03 (m, 1H), 6.86-6.77 (m, 1H), 4.81-4.70 (m, 0.5H), 4.28-4.20 (m, 0.5H), 4.18-4.10 (m, 0.5H), 3.91- 3.81 (m, 0.5H), 3.50 (dd, J = 13.4, 10.2 Hz, 0.5H), 3.12-3.04 (m, 0.5H), 2.99-2.89 (m, 0.5H), 2.88-2.70 (m, 1.5H), 2.27-2.18 (m, 1H), 2.08 (s, 3H), 2.04 (s, 3H), 1.93-1.73 (m, 2H), 1.66-1.41 (m, 1H); LCMS (Method B): t_{R} 2.84 min, 100%, MS (ESI) 448.2 (M + H)⁺ |
| 00075 | 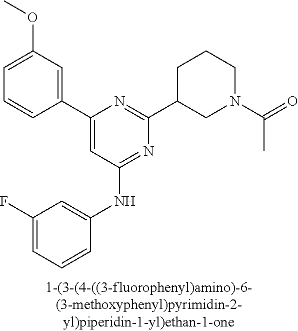 [SMILES: COc1cccc(-c2cc(Nc3cccc(F)c3)nc(C3CCCN(C(C)=O)C3)n2)c1] | ¹H-NMR (400 MHz, DMSO-d6) mixture of rotamers δ 9.89 (d, J = 10.5 Hz, 1H), 7.90 (t, J = 12.7 Hz, 1H), 7.63- 7.58 (m, 2H), 7.50-7.32 (m, 3H), 7.14-7.07 (m, 2H), 6.83 (t, J = 8.5 Hz, 1H), 4.79-4.68 (m, 0.5H), 4.25- 4.10 (m, 1H), 3.89-3.81 (m, 3.5H), 3.60-3.45 (m, 0.5H), 3.09 (t, J = 12.4 Hz, 0.5H), 3.00-2.91 (m, 0.5H), 2.90- 2.72 (m, 1.5H), 2.30-2.18 (m, 1H), 2.04 (s, 3H), 1.96-1.72 (m, 2H), 1.66- 1.39 (m, 1H); LCMS (Method D): t_{R} 3.71 min, 98%, MS (ESI) 421.2 (M + H)⁺ |
| 00076 | 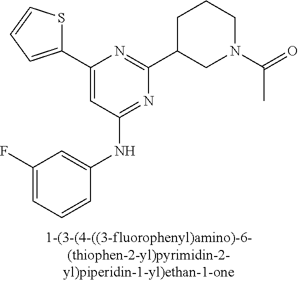 [SMILES: CC(=O)N1CCCC(c2nc(Nc3cccc(F)c3)cc(-c3cccs3)n2)C1] | ¹H-NMR (400 MHz, DMSO-d6) mixture of rotamers δ 9.89 (d, J = 11.0 Hz, 1H), 7.92-7.81 (m, 1H), 7.81- 7.77 (m, 1H), 7.77-7.73 (m, 1H), 7.43-7.31 (m, 2H), 7.25-7.19 (m, 1H), 7.03-6.98 (m, 1H), 6.87-6.78 (m, 1H), 4.86-4.58 (m, 0.5H), 4.21- 4.06 (m, 1H), 3.85 (d, J = 13.6 Hz, 0.5H), 3.54-3.45 (m, 0.5H), 3.11- 3.01 (m, 0.5H), 2.90 (m, J = 9.9, 4.9 Hz, 0.5H), 2.85-2.65 (m, 1.5H), 2.24- 2.14 (m, 1H), 2.04 (s, 3H), 1.90- 1.71 (m, 2H), 1.65-1.39 (m, 1H); LCMS (Method B): t_{R} 3.53 min, 100%, MS (ESI) 397.1 (M + H)⁺ |
| 00077 | 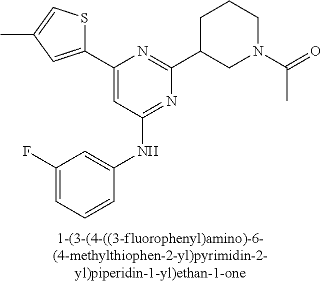 [SMILES: CC(=O)N1CCCC(c2nc(Nc3cccc(F)c3)cc(-c3cc(C)cs3)n2)C1] | ¹H-NMR (400 MHz, DMSO-d6) mixture of rotamers δ 9.86 (d, J = 10.8 Hz, 1H), 7.90-7.79 (m, 1H), 7.61 (d, J = 3.9 Hz, 1H), 7.42-7.31 (m, 3H), 6.94 (d, J = 3.5 Hz, 1H), 6.86-6.78 (m, 1H), 4.76-4.67 (m, 0.5H), 4.21- 4.13 (m, 0.5H), 4.13-4.05 (m, 0.5H), 3.90-3.80 (m, 0.5H), 3.53-3.44 (m, 0.5H), 2.94-2.84 (m, 0.5H), 2.84- 2.61 (m, 1.5H), 2.27 (s, 3H), 2.23- 2.14 (m, 1H), 2.04 (s, 3H), 1.91-1.70 (m, 2H), 1.65-1.37 (m, 1H); LCMS (Method B): t_{R} 3.66 min, 100%, MS (ESI) 411.1 (M + H)⁺ |
| 00078 | 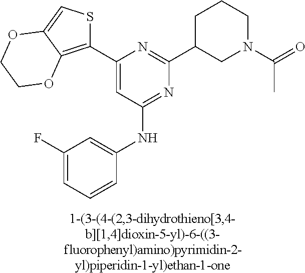 [SMILES: CC(=O)N1CCCC(c2nc(Nc3cccc(F)c3)cc(-c3scc4c3OCCO4)n2)C1] | ¹H-NMR (400 MHz, DMSO-d6) mixture of rotamers δ 9.91 (d, J = 11.5 Hz, 1H), 7.88 (t, J = 14.0 Hz, 1H), 7.44- 7.38 (m, 1H), 7.37-7.30 (m, 1H), 7.18 (d, J = 2.6 Hz, 1H), 6.83 (d, J = 1.3 Hz, 1H), 6.80 (t, J = 8.4 Hz, 1H), 4.78-4.64 (m, 0.5H), 4.48-4.40 (m, 2H), 4.35-4.25 (m, 2H), 4.19-4.05 (m, 1H), 3.92-3.73 (m, 0.5H), 3.55- 3.42 (m, 0.5H), 3.11-2.99 (m, 0.5H), 2.90-2.82 (m, 0.5H), 2.81-2.59 (m, 1.5H), 2.21-2.12 (m, 1H), 2.03 (s, 3H), 1.87-1.70 (m, 2H), 1.65-1.38 (m, 1H); LCMS (Method D): t_{R} 3.73 min, 97%, MS (ESI) 455.1 (M + H)⁺ |
| 00079 | 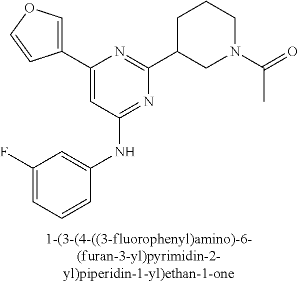 [SMILES: CC(=O)N1CCCC(c2nc(Nc3cccc(F)c3)cc(-c3ccoc3)n2)C1] | ¹H-NMR (400 MHz, DMSO-d6) mixture of rotamers δ 9.83 (d, J = 10.2 Hz, 1H), 8.36 (d, J = 8.7 Hz, 1H), 7.99- 7.76 (m, 2H), 7.46-7.27 (m, 2H), 7.02-6.93 (m, 1H), 6.84-6.78 (m, 2H), 4.78-4.54 (m, 0.5H), 4.25-4.02 (m, 1H), 3.85 (d, J = 13.5 Hz, 0.5H), 3.58-3.42 (m, 0.5H), 3.06 (t, J = 12.0 Hz, 0.5H), 2.94-2.64 (m, 2H), 2.25- 2.11 (m, 1H), 2.04 (s, 3H), 1.93-1.71 (m, 2H), 1.65-1.38 (m, 1H); LCMS (Method D): t_{R} 3.48 min, 99%, MS (ESI) 381.1 (M + H)⁺ |
| 00080 | 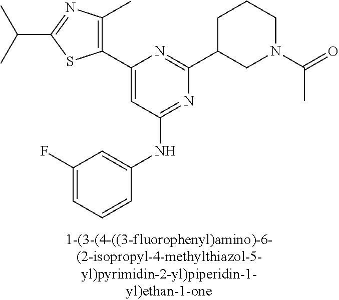 [SMILES: CC(=O)N1CCCC(c2nc(Nc3cccc(F)c3)cc(-c3sc(C(C)C)nc3C)n2)C1] | ¹H-NMR (400 MHz, DMSO-d6) mixture of rotamers δ 9.94 (d, J = 11.0 Hz, 1H), 7.90 (t, J = 13.7 Hz, 1H), 7.43- 7.33 (m, 2H), 6.96 (s, 1H), 6.90- 6.79 (m, 1H), 4.77-4.69 (m, 0.5H), 4.28-4.07 (m, 1H), 3.92-3.82 (m, 0.5H), 3.46-3.38 (m, 0.5H), 3.29- 3.20 (m, 1H), 3.11-3.01 (m, 0.5H), 2.95-2.84 (m, 0.5H), 2.83-2.69 (m, 1.5H), 2.66 (s, 3H), 2.23-2.15 (m, 1H), 2.04 (s, 3H), 1.90-1.70 (m, 2H), 1.65-1.41 (m, 1H), 1.36 (s, 3H), 1.34 (s, 3H); LCMS (Method D): t_{R} 3.83 min, 98%, MS (ESI) 454.2 (M + H)⁺ |
| 00081 | 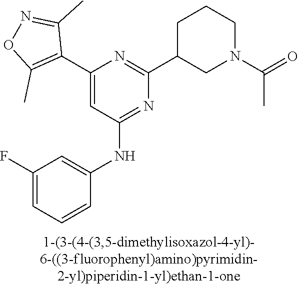 [SMILES: CC(=O)N1CCCC(c2nc(Nc3cccc(F)c3)cc(-c3c(C)noc3C)n2)C1] | ¹H-NMR (400 MHz, DMSO-d6) mixture of rotamers δ 9.91 (s, 1H), 7.96-7.82 (m, 1H), 7.44-7.26 (m, 2H), 6.91-6.77 (m, 2H), 4.77 (d, J = 11.1 Hz, 0.5H), 4.38-4.11 (m, 1H), 3.85 (d, J = 13.5 Hz, 0.5H), 3.43- 3.34 (m, 0.5H), 3.05 (t, J = 12.3 Hz, 0.5H), 2.98-2.86 (m, 0.5H), 2.84- 2.64 (m, 4.5H), 2.45 (d, J = 3.0 Hz, 3H), 2.29-2.16 (m, 1H), 2.04 (s, 3H), 1.89-1.69 (m, 2H), 1.65-1.38 (m, 1H); LCMS (Method D): t_{R} 3.47 min, 96%, MS (ESI) 410.2 (M + H)⁺ |
| 00082 | 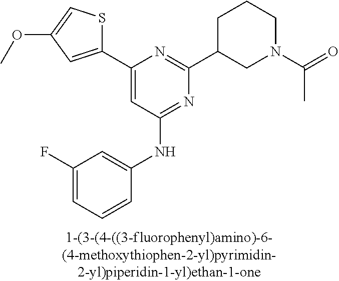 | ¹H-NMR (400 MHz, DMSO-d6) mixture of rotamers δ 9.88 (d, J = 11.2 Hz, 1H), 7.89-7.79 (m, 1H), 7.45- 7.31 (m, 3H), 6.95 (d, J = 3.3 Hz, 1H), 6.83 (s, 2H), 4.71 (d, J = 12.4 Hz, 0.5H), 4.24-4.00 (m, 1H), 3.89-3.82 (m, 0.5H), 3.79 (s, 3H), 3.52-3.44 (m, 0.5H), 3.06 (t, J = 12.4 Hz, 0.5H), 2.94-2.85 (m, 0.5H), 2.84-2.65 (m, 1.5H), 2.22-2.13 (m, 1H), 2.04 (s, 3H), 1.90-1.71 (m, 2H), 1.64-1.41 (m, 1H); LCMS (Method B): t_{R} 3.61 min, 97%, MS (ESI) 421.1 (M + H)⁺ |

The following further compounds were prepared using procedures analogous to Example 1.

| Compound | | |
|---|---|---|
| # | Structure and compound name | Analytical data |
| 00083 | 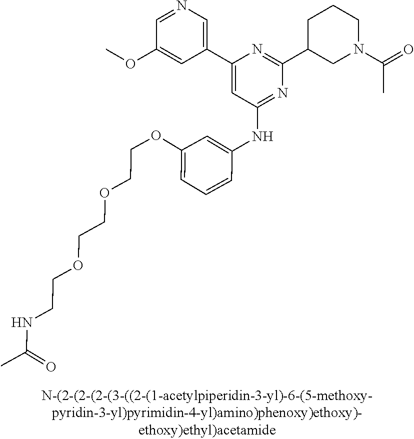 [SMILES: COc1cncc(-c2cc(Nc3cccc(OCCOCCOCCNC(C)=O)c3)nc(C3CCCN(C(C)=O)C3)n2)c1] | 1H-NMR (400 MHz, DMSO-d6) mixture of rotamers δ 9.78 (d, J = 8.3 Hz, 1H), 8.79 (dd, J = 5.8, 1.7 Hz, 1H), 8.43 (t, J = 2.6 Hz, 1H), 8.02-7.80 (m, 2H), 7.61 (s, 1H), 7.28-7.19 (m, 2H), 7.17 (d, J = 3.8 Hz, 1H), 6.70-6.54 (m, 1H), 4.72 (d, J = 13.0 Hz, 0.5H), 4.22 (d, J = 13.1 Hz, 0.5H), 4.16-4.07 (m, 2.5H), 3.93 (s, 3H), 3.85 (d, J = 13.2 Hz, 0.5H), 3.79-3.72 (m, 2H), 3.61 (t, J = 4.6 Hz, 2H), 3.54 (dd, J = 7.2, 3.2 Hz, 2.5H), 3.40 (t, J = 5.7 Hz, 2H), 3.18 (q, J = 5.8 Hz, 2H), 3.07 (s, 0.5H), 3.00- 2.85 (m, 1H), 2.83-2.71 (m, 1H), 2.22 (d, J = 12.7 Hz, 1H), 2.04 (d, J = 1.4 Hz, 3H), 1.96-1.71 (m, 5H), 1.67-1.37 (m, 1H); LCMS (Method D): tR 2.94 min, 100%, MS (ESI) 593.2 (M + H)+ |
| 00084 | 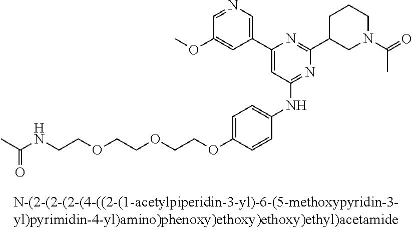 [SMILES: COc1cncc(-c2cc(Nc3ccc(OCCOCCOCCNC(C)=O)cc3)nc(C3CCCN(C(C)=O)C3)n2)c1] | ¹H-NMR (400 MHz, DMSO-d6) mixture of rotamers δ 9.59 (d, J = 8.3 Hz, 1H), 8.76 (dd, J = 5.8, 1.8 Hz, 1H), 8.41 (t, J = 2.6 Hz, 1H), 7.88 (t, J = 2.4 Hz, 2H), 7.62 (d, J = 8.3 Hz, 2H), 7.06 (d, J = 4.4 Hz, 1H), 6.99-6.89 (m, 2H), 4.68 (d, J = 13.6 Hz, 0.5H), 4.18 (d, J = 12.9 Hz, 0.5H), 4.08 (dd, J = 5.7, 3.7 Hz, 2.5H), 3.92 (s, 3H), 3.84 (d, J = 13.6 Hz, 0.5H), 3.79- 3.69 (m, 2H), 3.60 (dd, J = 6.2, 3.5 Hz, 2H), 3.54 (dd, J = 6.2, 3.7 Hz, 2.5H), 3.46-3.39 (m, 2H), |
| | | 3.22-3.13 (m, 2H), 3.08 (t, J = |
| | | 12.3 Hz, 0.5H), 2.94-2.69 (m, |
| | | 2H), 2.18 (d, J = 12.6 Hz, 1H), |
| | | 2.03 (d, J = 3.3 Hz, 3H), 1.79 (s, |
| | | 5H), 1.65-1.39 (m, 1H); LCMS |
| | | (Method D): t_{R} 2.88 min, 100%, |
| | | MS (ESI) 593.3 (M + H)⁺ |
| 00085 | 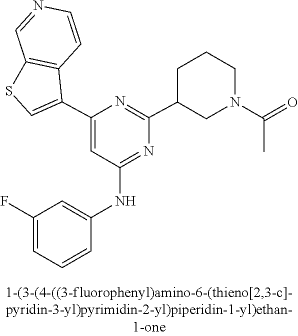 [SMILES: CC(=O)N1CCCC(c2nc(Nc3cccc(F)c3)cc(-c3csc4cnccc34)n2)C1] | ¹H-NMR (400 MHz, DMSO-d6) mixture of rotamers δ 10.00 (d, J = 11.6 Hz, 1H), 9.37 (s, 1H), 8.74 (d, J = 5.8 Hz, 1H), 8.65- 8.43 (m, 2H), 7.99-7.81 (m, 1H), 7.51-7.30 (m, 2H), 7.15 (d, J = 5.8 Hz, 1H), 6.85 (t, J = 8.6 Hz, 1H), 4.81 (d, J = 11.0 Hz, 0.5H), 4.39-4.12 (m, 1H), 3.94-3.78 (m, 0.5H), 3.53-3.42 (m, 0.5H), 3.16- 3.05 (m, 0.5H), 3.05-2.95 (m, 0.5H), 2.93-2.64 (m, 1.5H), 2.32- 2.20 (m, 1H), 2.06 (d, J = 3.0 Hz, 3H), 1.96-1.75 (m, 2H), 1.72- 1.41 (m, 1H); LCMS (Method B): t_{R} 2.80 min, 97%, MS (ESI) 448.1 (M + H)⁺ |
| 00086 | 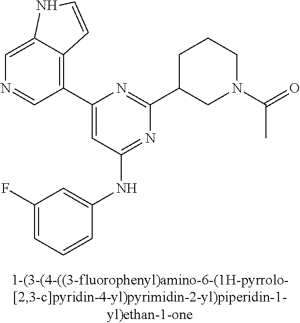 [SMILES: CC(=O)N1CCCC(c2nc(Nc3cccc(F)c3)cc(-c3cncc4[nH]ccc34)n2)C1] | ¹H-NMR (400 MHz, DMSO-d6) mixture of rotamers δ 11.92 (s, 1H), 9.92 (d, J = 10.2 Hz, 1H), 8.85 (s, 1H), 8.73 (d, J = 4.9 Hz, 1H), 8.02-7.89 (m, 1H), 7.86- 7.74 (m, 1H), 7.45-7.34 (m, 2H), 7.29 (d, J = 4.7 Hz, 1H), 7.10- 7.02 (m, 1H), 6.90-6.76 (m, 1H), 4.83 (d, J = 10.9 Hz, 0.5H), 4.32- 4.15 (m, 1H), 3.95-3.79 (m, 0.5H), 3.56-3.45 (m, 0.5H), 3.15- 3.05 (m, 0.5H), 3.04-2.95 (m, 0.5H), 2.91-2.69 (m, 1.5H), 2.32- 2.25 (m, 1H), 2.06 (d, J = 2.0 Hz, 3H), 1.97-1.75 (m, 2H), 1.69-1.42 (m, 1H); LCMS (Method B): t_{R} 2.63 min, 98%, MS (ESI) 431.1 (M + H)⁺ |
| 00087 | 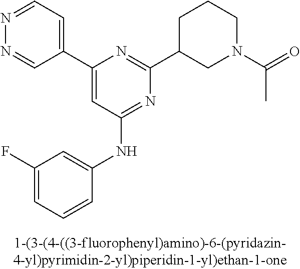 [SMILES: CC(=O)N1CCCC(c2nc(Nc3cccc(F)c3)cc(-c3ccnnc3)n2)C1] | ¹H-NMR (400 MHz, DMSO-d6) mixture of rotamers δ 10.19 (s, 1H), 9.87-9.73 (m, 1H), 9.48-9.38 (m, 1H), 8.28-8.16 (m, 1H), 7.95- 7.76 (m, 1H), 7.49-7.26 (m, 3H), 6.87 (t, J = 8.4 Hz, 1H), 4.78-4.67 (m, 0.5H), 4.30-4.10 (m, 1H), 3.85 (d, J = 13.3 Hz, 0.5H), 3.56- 3.45 (m, 0.5H), 3.15-3.05 (m, 0.5H), 3.05-2.97 (m, 0.5H), 2.95- 2.71 (m, 1.5H), 2.24 (s, 1H), 2.05 (s, 3H), 1.95-1.74 (m, 2H), 1.67- 1.41 (m, 1H); LCMS (Method D): t_{R} 3.03 min, 98%, MS (ESI) 393.1 (M + H)⁺ |
| 00088 | 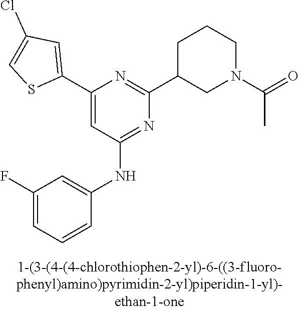 [SMILES: CC(=O)N1CCCC(c2nc(Nc3cccc(F)c3)cc(-c3cc(Cl)cs3)n2)C1] | ¹H-NMR (400 MHz, DMSO-d6) mixture of rotamers δ 9.98 (d, J = 10.9 Hz, 1H), 7.90-7.70 (m, 3H), 7.45-7.21 (m, 2H), 7.02 (d, J = 3.9 Hz, 1H), 6.92-6.77 (m, 1H), 4.71 (d, J = 11.8 Hz, 0.5H), 4.26- 4.00 (m, 1H), 3.85 (d, J = 13.6 Hz, 0.5H), 3.55-3.41 (m, 0.5H), 3.14-2.99 (m, 0.5H), 2.95-2.85 (m, 0.5H), 2.84-2.65 (m, 1.5H), 2.25-2.13 (m, 1H), 2.04 (s, 3H), 1.93-1.69 (m, 2H), 1.65-1.38 (m, 1H); LCMS (Method D): t_{R} 3.93 min, 96%, MS (ESI) 431.1 (M + H)⁺ |
| 00089 | 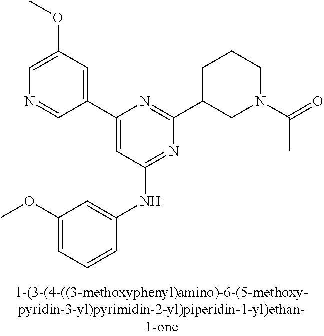 [SMILES: COc1cccc(Nc2cc(-c3cncc(OC)c3)nc(C3CCCN(C(C)=O)C3)n2)c1] | ¹H-NMR (400 MHz, DMSO-d6) mixture of rotamers δ 9.76 (d, J = 7.2 Hz, 1H), 8.84-8.73 (m, 1H), 8.43 (t, J = 2.6 Hz, 1H), 7.90 (t, J = 2.3 Hz, 1H), 7.66-7.56 (m, 1H), 7.30-7.12 (m, 3H), 6.65-6.58 (m, 1H), 4.78-4.69 (m, 0.5H), 4.24-4.16 (m, 0.5H), 4.16-4.07 (m, 0.5H), 3.93 (s, 3H), 3.90- 3.81 (m, 0.5H), 3.81-3.75 (m, 3H), 3.58-3.49 (m, 0.5H), 3.12- 3.02 (m, 0.5H), 2.99-2.71 (m, 2H), 2.26-2.16 (m, 1H), 2.07-2.00 (m, 3H), 1.97-1.69 (m, 2H), 1.66- 1.39 (m, 1H); LCMS (Method D): t_{R} 3.15 min, 99%, MS (ESI) 434.2 (M + H)⁺ |
| 00090 | 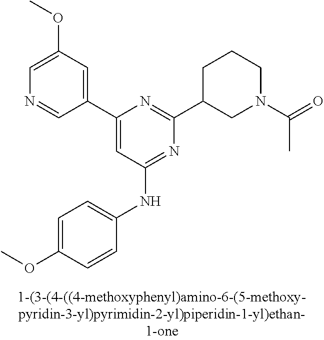 [SMILES: COc1ccc(Nc2cc(-c3cncc(OC)c3)nc(C3CCCN(C(C)=O)C3)n2)cc1] | ¹H-NMR (400 MHz, DMSO-d6) mixture of rotamers δ 9.58 (d, J = 7.0 Hz, 1H), 8.81-8.73 (m, 1H), 8.41 (t, J = 2.6 Hz, 1H), 7.88 (t, J = 2.4 Hz, 1H), 7.63 (d, J = 8.4 Hz, 2H), 7.05 (d, J = 3.9 Hz, 1H), 6.99-6.91 (m, 2H), 4.73-4.63 (m, 0.5H), 4.23-4.14 (m, 0.5H), 4.12-4.04 (m, 0.5H), 3.92 (s, 3H), 3.88-3.80 (m, 0.5H), 3.75 (s, 3H), 3.56-3.48 (m, 0.5H), 3.14-3.03 (m, 0.5H), 2.95-2.65 (m, 2H), 2.24-2.14 (m, 1H), 2.07-1.98 (m, 3H), 1.93-1.69 (m, 2H), 1.63-1.38 (m, 1H); LCMS (Method D): t_{R} 3.09 min, 98%, MS (ESI) 434.2 (M + H)⁺ |
| 00091 | 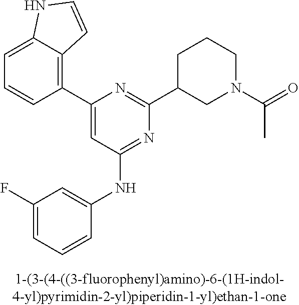 [SMILES: CC(=O)N1CCCC(c2nc(Nc3cccc(F)c3)cc(-c3cccc4[nH]ccc34)n2)C1] | ¹H-NMR (400 MHz, DMSO-d6) mixture of rotamers δ 11.38 (s, 1H), 9.81 (d, J = 10.0 Hz, 1H), 8.22 (s, 1H), 7.99-7.88 (m, 1H), 7.73-7.64 (m, 2H), 7.50 (t, J = 2.7 Hz, 1H), 7.44-7.33 (m, 2H), 7.15 (d, J = 3.6 Hz, 1H), 6.81 (t, J = 8.4 Hz, 1H), 6.52-6.48 (m, 1H), 4.82 (d, J = 11.9 Hz, 0.5H), 4.34-4.13 (m, 1H), 3.94-3.84 (m, 0.5H), 3.58-3.46 (m, 0.5H), 3.14-3.03 (m, 0.5H), 3.01-2.91 (m, 0.5H), 2.90-2.69 (m, 1.5H), 2.27 (s, 1H), 2.06 (d, J = 1.7 Hz, 3H), 1.97- 1.74 (m, 2H), 1.71-1.42 (m, 1H); UPLC (Method A): t_{R} 1.64 min, 98%, MS (ESI) 430.0 (M + H)⁺ |
| 00092 | 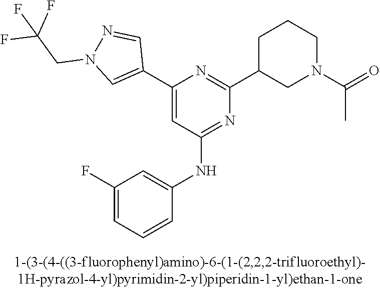 [SMILES: CC(=O)N1CCCC(c2nc(Nc3cccc(F)c3)cc(-c3cnn(CC(F)(F)F)c3)n2)C1] | ¹H-NMR (400 MHz, DMSO-d6) mixture of rotamers δ 9.94-9.68 (m, 1H), 8.47 (d, J = 4.0 Hz, 1H), 8.12 (d, J = 6.3 Hz, 1H), 7.94-7.77 (m, 1H), 7.46-7.29 (m, 2H), 6.91- 6.73 (m, 2H), 5.23 (q, J = 9.0 Hz, 2H), 4.72 (d, J = 13.1 Hz, 0.5H), 4.30-4.05 (m, 1H), 3.86 (d, J = 13.4 Hz, 0.5H), 3.59-3.41 (m, 0.5H), 3.12-2.99 (m, 0.5H), 2.94- 2.64 (m, 2H), 2.27-2.14 (m, 1H), 2.04 (s, 3H), 1.92-1.70 (m, 2H), 1.66-1.36 (m, 1H); UPLC (Method A): t_{R} 1.51 min, 100%, MS (ESI) 463.0 (M + H)⁺ |
| 00093 | 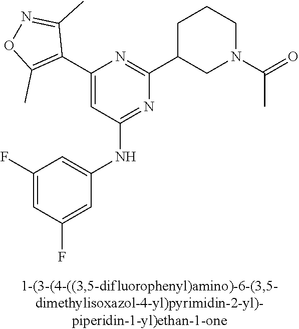 [SMILES: CC(=O)N1CCCC(c2nc(Nc3cc(F)cc(F)c3)cc(-c3c(C)noc3C)n2)C1] | ¹H-NMR (400 MHz, DMSO-d6) mixture of rotamers δ 10.14 (d, J = 16.1 Hz, 1H), 7.58-7.48 (m, 2H), 6.88-6.79 (m, 2H), 4.82-4.72 (m, 0.5H), 4.37-4.15 (m, 1H), 3.85 (d, J = 13.4 Hz, 0.5H), 3.11-3.00 (m, 0.5H), 2.99-2.88 (m, 1H), 2.83- 2.73 (m, 1H), 2.67 (d, J = 2.9 Hz, 3H), 2.65-2.59 (m, 0.5H), 2.45 (d, J = 3.1 Hz, 3H), 2.29-2.16 (m, 1H), 2.04 (d, J = 4.5 Hz, 3H), 1.89-1.71 (m, 2H), 1.66-1.38 (m, 1H); LCMS (Method B): t_{R} 3.52 min, 100%, MS (ESI) 428.2 (M + H)⁺ |
| 00094 | 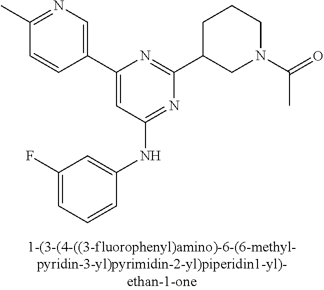 [SMILES: CC(=O)N1CCCC(c2nc(Nc3cccc(F)c3)cc(-c3ccc(C)nc3)n2)C1] | ¹H-NMR (400 MHz, DMSO-d6) mixture of rotamers δ 9.96 (d, J = 11.1 Hz, 1H), 9.08 (dd, J = 5.8, 2.4 Hz, 1H), 8.28 (dt, J = 8.1, 2.9 Hz, 1H), 7.97-7.81 (m, 1H), 7.49- 7.31 (m, 3H), 7.15 (d, J = 4.6 Hz, 1H), 6.84 (t, J = 8.4 Hz, 1H), 4.81- 4.68 (m, 0.5H), 4.23 (dd, J = 10.8, 6.7 Hz, 0.5H), 4.14 (dd, J = 13.4, 3.9 Hz, 0.5H), 3.85 (d, J = 13.4 Hz, 0.5H), 3.50 (dd, J = 13.4, 10.0 Hz, 0.5H), 3.14-3.03 (m, 0.5H), 3.02-2.91 (m, 0.5H), 2.91- 2.81 (m, 0.5H), 2.81-2.72 (m, 1H), 2.55 (s, 3H), 2.27-2.18 (m, 1H), 2.04 (s, 3H), 1.96-1.71 (m, 2H), 1.67-1.41 (m, 1H); LCMS (Method D): t_{R} 3.24 min, 100%, MS (ESI) 406.2 (M + H)⁺ |
| 00095 | 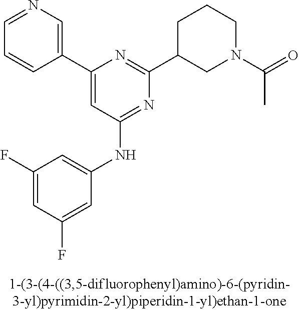 [SMILES: CC(=O)N1CCCC(c2nc(Nc3cc(F)cc(F)c3)cc(-c3cccnc3)n2)C1] | ¹H-NMR (400 MHz, DMSO-d6) mixture of rotamers δ 10.19 (d, J = 11.4 Hz, 1H), 9.30-9.14 (m, 1H), 8.76-8.67 (m, 1H), 8.46-8.32 (m, 1H), 7.66-7.49 (m, 3H), 7.19 (d, J = 5.1 Hz, 1H), 6.92-6.74 (m, 1H), 4.81-4.71 (m, 0.5H), 4.29- 4.13 (m, 1H), 3.86 (d, J = 13.3 Hz, 0.5H), 3.55-3.45 (m, 0.5H), 3.14- 3.04 (m, 0.5H), 3.03-2.94 (m, 0.5H), 2.93-2.71 (m, 1.5H), 2.30- 2.20 (m, 1H), 2.05 (s, 3H), 1.95- 1.73 (m, 2H), 1.68-1.41 (m, 1H); LCMS (Method D): t_{R} 3.27 min, 100%, MS (ESI) 410.1 (M + H)⁺ |
| 00096 | 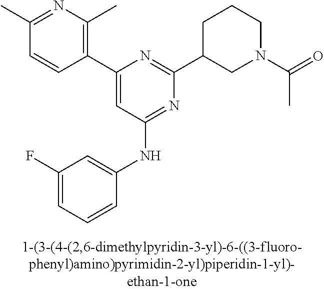 [SMILES: CC(=O)N1CCCC(c2nc(Nc3cccc(F)c3)cc(-c3ccc(C)nc3C)n2)C1] | ¹H-NMR (400 MHz, DMSO-d6) mixture of rotamers δ 9.91 (d, J = 10.3 Hz, 1H), 7.94-7.83 (m, 1H), 7.76 (dd, J = 7.8, 2.1 Hz, 1H), 7.44-7.32 (m, 2H), 7.22 (d, J = 7.6 Hz, 1H), 6.88-6.81 (m, 1H), 6.80 (d, J = 5.8 Hz, 1H), 4.74 (d, J = 11.3 Hz, 0.5H), 4.24 (d, J = 12.9 Hz, 0.5H), 4.17-4.07 (m, 0.5H), 3.85 (d, J = 13.3 Hz, 0.5H), 3.42 (dd, J = 13.5, 10.4 Hz, 0.5H), 3.12-2.99 (m, 0.4H), 2.99-2.87 (m, 0.5H), 2.85-2.68 (m, 1.5H), 2.56 (d, J = 3.1 Hz, 3H), 2.50 (d, J = 3.1 Hz, 3H), 2.21 (d, J = 12.6 Hz, 1H), 2.03 (d, J = 3.4 Hz, 3H), 1.92-1.69 (m, 2H), 1.65-1.37 (m, 1H); LCMS (Method D): t_{R} 3.187 |
| | | min, 100%, MS (ESI) 420.2 |
| | | (M + H)⁺ |
| 00097 | 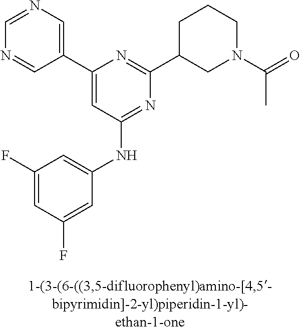 [SMILES: CC(=O)N1CCCC(c2nc(Nc3cc(F)cc(F)c3)cc(-c3cncnc3)n2)C1] | ¹H-NMR (400 MHz, DMSO-d6) mixture of rotamers δ 10.37 (s, 1H), 9.40 (d, J = 7.7 Hz, 2H), 9.33 (d, J = 1.9 Hz, 1H), 7.66-7.47 (m, 2H), 7.28 (d, J = 8.4 Hz, 1H), 6.91-6.78 (m, 1H), 4.79-4.60 (m, 0.5H), 4.30-4.06 (m, 1H), 3.84 (d, J = 13.5 Hz, 0.5H), 3.58-3.46 (m, 0.5H), 3.16-2.71 (m, 2.5H), 2.30- 2.19 (m, 1H), 2.05 (d, J = 2.4 Hz, 3H), 1.95-1.71 (m, 2H), 1.70- 1.40 (m, 1H); LCMS (Method D): t_{R} 3.14 min, 99%, MS (ESI) 411.1 (M + H)⁺ |
| 00098 | 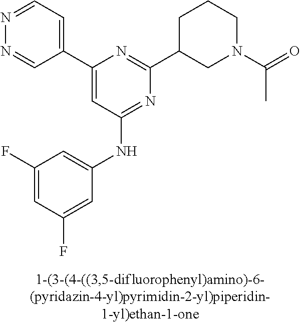 [SMILES: CC(=O)N1CCCC(c2nc(Nc3cc(F)cc(F)c3)cc(-c3ccnnc3)n2)C1] | ¹H-NMR (400 MHz, DMSO-d6) mixture of rotamers δ 10.32 (d, J = 10.4 Hz, 1H), 9.89-9.73 (m, 1H), 9.53-9.35 (m, 1H), 8.29-8.15 (m, 1H), 7.65-7.49 (m, 2H), 7.32 (d, J = 5.6 Hz, 1H), 6.98-6.78 (m, 1H), 4.79-4.67 (m, 0.5H), 4.30- 4.09 (m, 1H), 3.85 (d, J = 13.4 Hz, 0.5H), 3.58-3.44 (m, 0.5H), 3.15- 3.06 (m, 0.5H), 3.06-2.97 (m, 0.5H), 2.97-2.70 (m, 1.5H), 2.30- 2.19 (m, 1H), 2.05 (s, 3H), 1.95- 1.73 (m, 2H), 1.67-1.42 (m, 1H); LCMS (Method D): t_{R} 3.08 min, 99%, MS (ESI) 411.1 (M + H)⁺ |
| 00099 | 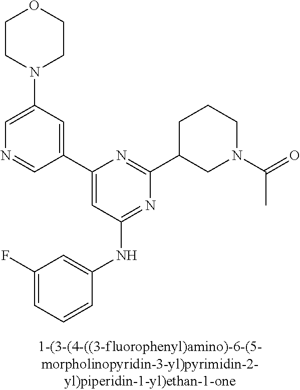 [SMILES: CC(=O)N1CCCC(c2nc(Nc3cccc(F)c3)cc(-c3cncc(N4CCOCC4)c3)n2)C1] | ¹H-NMR (400 MHz, DMSO-d6) mixture of rotamers δ 9.95 (d, J = 10.7 Hz, 1H), 8.63 (dd, J = 6.7, 1.8 Hz, 1H), 8.45 (t, J = 2.5 Hz, 1H), 7.95-7.80 (m, 2H), 7.46-7.32 (m, 2H), 7.16 (d, J = 5.4 Hz, 1H), 6.88-6.78 (m, 1H), 4.77-4.63 (m, 0.5H), 4.23-4.08 (m, 1H), 3.79 (t, J = 4.8 Hz, 4.5H), 3.53 (dd, J = 13.4, 9.9 Hz, 0.5H), 3.30-3.23 (m, 4H), 3.08 (d, J = 11.8 Hz, 0.5H), 3.02-2.87 (m, 1H), 2.86-2.74 (m, 1H), 2.28-2.15 (m, 1H), 2.04 (s, 3H), 1.96-1.71 (m, 2H), 1.68-1.40 (m, 1H); LCMS (Method B): t_{R} 2.90 min, 99%, MS (ESI) 477.2 (M + H)⁺ |
| 00100 | 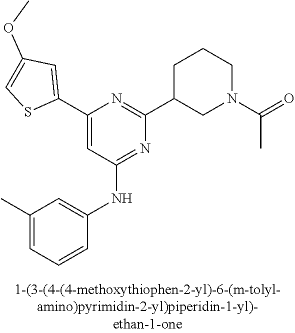 [SMILES: COc1csc(-c2cc(Nc3cccc(C)c3)nc(C3CCCN(C(C)=O)C3)n2)c1] | ¹H-NMR (400 MHz, DMSO-d6) mixture of rotamers δ 9.58 (d, J = 8.8 Hz, 1H), 7.59 (s, 1H), 7.48 (d, J = 8.1 Hz, 1H), 7.39 (dd, J = 5.0, 1.7 Hz, 1H), 7.21 (td, J = 7.8, 1.9 Hz, 1H), 6.91 (d, J = 3.4 Hz, 1H), 6.87-6.78 (m, 2H), 4.82-4.68 (m, 0.5H), 4.05 (dd, J = 13.5, 3.9 Hz, 0.5H), 3.85 (d, J = 13.4 Hz, 0.5H), 3.79 (s, 3H), 3.50 (dd, J = 13.5, 9.7 Hz, 0.5H), 3.10-2.99 (m, 0.5H), 2.90-2.71 (m, 1.5H), 2.71- 2.62 (m, 0.5H), 2.31 (d, J = 2.7 Hz, 3H), 2.23-2.10 (m, 1H), 2.03 (s, 3H), 1.93-1.70 (m, 2H), 1.62- 1.36 (m, 1H); LCMS (Method B): t_{R} 3.57 min, 99%, MS (ESI) 423.2 (M + H)+ |
| 00101 | 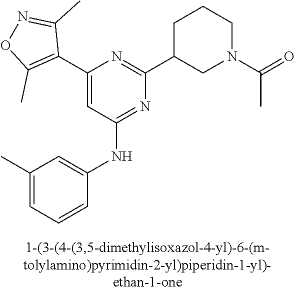 [SMILES: CC(=O)N1CCCC(c2nc(Nc3cccc(C)c3)cc(-c3c(C)noc3C)n2)C1] | ¹H-NMR (400 MHz, DMSO-d6) mixture of rotamers δ 9.60 (d, J = 8.5 Hz, 1H), 7.59 (d, J = 7.1 Hz, 1H), 7.54-7.45 (m, 1H), 7.22 (td, J = 7.8, 2.9 Hz, 1H), 6.85 (d, J = 7.6 Hz, 1H), 6.79 (d, J = 4.9 Hz, 1H), 4.80 (d, J = 11.7 Hz, 0.5H), 4.28 (d, J = 13.0 Hz, 0.5H), 4.14 (d, J = 14.7 Hz, 0.5H), 3.85 (d, J = 13.5 Hz, 0.5H), 3.44-3.34 (m, 0.5H), 3.08-2.99 (m, 0.5H), 2.92- 2.81 (m, 0.5H), 2.79-2.62 (m, 4.5H), 2.44 (d, J = 2.6 Hz, 3H), 2.31 (d, J = 2.7 Hz, 3H), 2.26- 2.16 (m, 1H), 2.03 (s, 3H), 1.88- 1.70 (m, 2H), 1.64-1.35 (m, 1H); LCMS (Method B): t_{R} 3.18 min, 99%, MS (ESI) 406.2 (M + H)⁺ |
| 00102 | 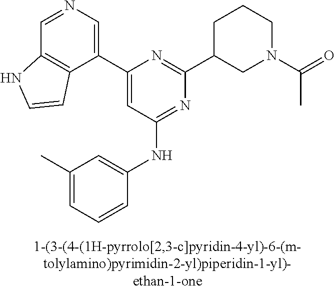 [SMILES: CC(=O)N1CCCC(c2nc(Nc3cccc(C)c3)cc(-c3cncc4[nH]ccc34)n2)C1] | ¹H-NMR (400 MHz, DMSO-d6) mixture of rotamers δ 11.89 (s, 1H), 9.61 (d, J = 8.0 Hz, 1H), 8.84 (s, 1H), 8.72 (d, J = 4.9 Hz, 1H), 8.14 (s, 0.5H), 7.79 (d, J = 3.0 Hz, 1H), 7.65 (d, J = 4.9 Hz, 1H), 7.59-7.51 (m, 1H), 7.24 (dd, J = 7.8, 5.1 Hz, 2H), 7.04 (d, J = 3.0 Hz, 1H), 6.85 (d, J = 7.4 Hz, 1H), 4.86 (dd, J = 12.1, 3.2 Hz, 0.5H), 4.25 (m, 0.5H), 4.18 (dd, J = 13.4, 4.0 Hz, 0.5H), 3.88 (dd, J = 13.2, 3.6 Hz, 0.5H), 3.51 (dd, J = 13.4, 10.3 Hz, 0.5H), 3.08 (m, 0.5H), 2.96 (m, 0.5H), 2.89-2.71 (m, 1.5H), 2.37-2.22 (m, 4H), 2.05 (s, 3H), 2.00-1.73 (m, 2H), 1.68-1.42 (m, 1H); LCMS (Method B): t_{R} |
| | | 2.61 min, 99%, MS (ESI) 427.2 |
| | | (M + H)⁺ |
| 00103 | 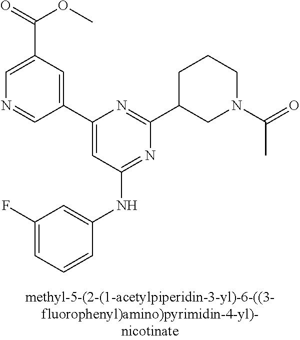 [SMILES: COC(=O)c1cncc(-c2cc(Nc3cccc(F)c3)nc(C3CCCN(C(C)=O)C3)n2)c1] | ¹H-NMR (400 MHz, DMSO-d6) mixture of rotamers δ 10.04 (d, J = 10.2 Hz, 1H), 9.44 (dd, J = 7.8, 2.2 Hz, 1H), 9.20 (t, J = 1.7 Hz, 1H), 8.82 (q, J = 2.1 Hz, 1H), 7.95-7.84 (m, 1H), 7.47-7.34 (m, 2H), 7.28 (d, J = 4.5 Hz, 1H), 6.90-6.81 (m, 1H), 4.82-4.70 (m, 0.5H), 4.31- 4.21 (m, 0.5H), 4.16 (dd, J = 14.2, 3.9 Hz, 0.5H), 3.95 (s, 3H), 3.86 (d, J = 13.6 Hz, 0.5H), 3.51 (dd, J = 13.4, 10.3 Hz, 0.5H), 3.15- 3.04 (m, 0.5H), 3.04-2.94 (m, 0.5H), 2.92-2.63 (m, 1.5H), 2.29- 2.19 (m, 1H), 2.05 (s, 3H), 1.95- 1.72 (m, 2H), 1.69-1.39 (m, 1H); LCMS (Method B): t_{R} 3.35 min, 98%, MS (ESI) 450.2 (M + H)⁺ |
| 00104 | 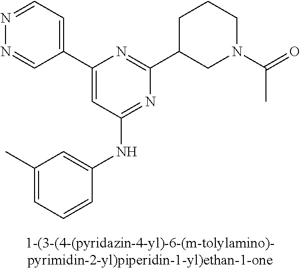 [SMILES: CC(=O)N1CCCC(c2nc(Nc3cccc(C)c3)cc(-c3ccnnc3)n2)C1] | ¹H-NMR (400 MHz, DMSO-d6) mixture of rotamers δ 9.87 (d, J = 9.0 Hz, 1H), 9.78 (dt, J = 7.1, 1.8 Hz, 1H), 9.42 (ddd, J = 5.5, 2.7, 1.1 Hz, 1H), 8.20 (td, J = 5.1, 2.4 Hz, 1H), 7.62 (q, J = 5.8, 4.1 Hz, 1H), 7.58-7.48 (m, 1H), 7.31-7.20 (m, 2H), 6.89 (d, J = 7.4 Hz, 1H), 4.82-4.70 (m, 0.5H), 4.26-4.18 (m, 0.5H), 4.13 (dd, J = 13.5, 4.0 Hz, 0.5H), 3.85 (d, J = 13.3 Hz, 0.5H), 3.52 (dd, J = 13.4, 10.1 Hz, 0.5H), 3.14-3.03 (m, 0.5H), 3.00-2.83 (m, 1H), 2.83-2.72 (m, 1H), 2.33 (d, J = 2.4 Hz, 3H), 2.28-2.17 (m, 1H), 2.04 (d, J = 1.4 Hz, 3H), 1.96-1.72 (m, 2H), 1.66-1.38 (m, |
| | | 1H); LCMS (Method B): t_{R} 3.08 |
| | | min, 99%, MS (ESI) 389.2 |
| | | (M + H)⁺ |
| 00105 | 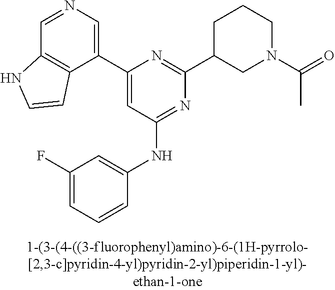 [SMILES: CC(=O)N1CCCC(c2nc(Nc3cccc(F)c3)cc(-c3cncc4[nH]ccc34)n2)C1] | ¹H-NMR (400 MHz, DMSO-d6) mixture of rotamers δ 11.75 (s, 1H), 9.08 (d, J = 7.0 Hz, 1H), 8.78 (s, 1H), 8.56 (d, J = 2.6 Hz, 1H), 7.71 (t, J = 2.8 Hz, 1H), 7.43-7.32 (m, 2H), 7.15-7.02 (m, 2H), 6.99- 6.88 (m, 2H), 6.83 (td, J = 8.4, 2.5 Hz, 1H), 4.63 (d, J = 12.4 Hz, 0.5H), 4.41 (d, J = 12.8 Hz, 0.5H), 4.02 (d, J = 13.4 Hz, 0.5H), 3.86 (d, J = 13.3 Hz, 0.5H), 3.42-3.35 (m, 0.5H), 3.07 (t, J = 12.3 Hz, 0.5H), 2.94-2.70 (m, 1.5H), 2.59 (t, J = 11.9 Hz, 0.5H), 2.11-2.00 (m, 4H), 1.93-1.72 (m, 2H), 1.64- 1.37 (m, 1H); LCMS (Method D): t_{R} 3.06 min, 96%, MS (ESI) 430.1 (M + H)+ |
| 00106 | 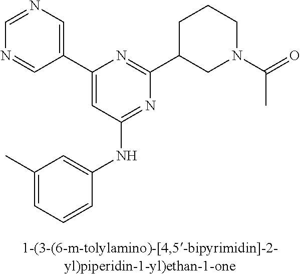 [SMILES: CC(=O)N1CCCC(c2nc(Nc3cccc(C)c3)cc(-c3cncnc3)n2)C1] | ¹H-NMR (400 MHz, DMSO-d6) mixture of rotamers δ 9.80 (d, J = 8.4 Hz, 1H), 9.36 (d, J = 6.9 Hz, 2H), 9.32 (d, J = 2.4 Hz, 1H), 7.66-7.49 (m, 2H), 7.24 (td, J = 7.8, 2.4 Hz, 1H), 7.19 (d, J = 5.4 Hz, 1H), 6.87 (d, J = 7.3 Hz, 1H), 4.81-4.67 (m, 0.5H), 4.20 (dd, J = 12.9, 4.2 Hz, 0.5H), 4.12 (dd, J = 13.6, 4.0 Hz, 0.5H), 3.85 (d, J = 13.3 Hz, 0.5H), 3.52 (dd, J = 13.5, 10.1 Hz, 0.5H), 3.14-3.02 (m, 0.5H), 2.99-2.83 (m, 1H), 2.83- 2.70 (m, 1H), 2.33 (d, J = 2.4 Hz, 3H), 2.27-2.17 (m, 1H), 2.04 (d, J = 1.4 Hz, 3H), 1.96-1.71 (m, 2H), 1.65-1.39 (m, 1H); LCMS |
| | | (Method B): t_{R} 2.98 min, 99%, |
| | | MS (ESI) 389.2 (M + H)⁺ |
| 00107 | 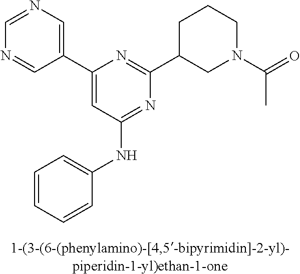 [SMILES: CC(=O)N1CCCC(c2nc(Nc3ccccc3)cc(-c3cncnc3)n2)C1] | ¹H-NMR (400 MHz, DMSO-d6) mixture of rotamers δ 9.88 (d, J = 9.0 Hz, 1H), 9.37 (d, J = 6.8 Hz, 2H), 9.32 (d, J = 2.4 Hz, 1H), 7.75 (d, J = 8.3 Hz, 2H), 7.41-7.31 (m, 2H), 7.21 (d, J = 5.1 Hz, 1H), 7.10-7.00 (m, 1H), 4.77-4.64 (m, 0.5H), 4.28-4.18 (m, 0.5H), 4.16- 4.07 (m, 0.5H), 3.59-3.44 (m, 0.5H), 3.19-3.01 (m, 0.5H), 2.99- 2.85 (m, 1H), 2.83-2.71 (m, 1H), 2.26-2.17 (m, 1H), 2.04 (d, J = 2.0 Hz, 3H), 1.95-1.72 (m, 2H), 1.67- 1.36 (m, 1H); LCMS (Method B): t_{R} 2.92 min, 100%, MS (ESI) 375.2 (M + H)⁺ |
| 00108 | 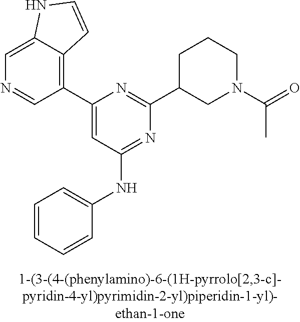 [SMILES: CC(=O)N1CCCC(c2nc(Nc3ccccc3)cc(-c3cncc4[nH]ccc34)n2)C1] | ¹H-NMR (400 MHz, DMSO-d6) mixture of rotamers δ 11.90 (s, 1H), 9.69 (d, J = 7.7 Hz, 1H), 8.84 (s, 1H), 8.72 (d, J = 4.4 Hz, 1H), 7.85-7.73 (m, 3H), 7.42-7.31 (m, 2H), 7.26 (d, J = 3.7 Hz, 1H), 7.09-7.00 (m, 2H), 4.90-4.71 (m, 0.5H), 4.32-4.09 (m, 1H), 3.95- 3.81 (m, 0.5H), 3.57-3.45 (m, 0.5H), 3.13-3.02 (m, 0.5H), 3.01- 2.64 (m, 2H), 2.31-2.21 (m, 1H), 2.05 (s, 3H), 1.98-1.74 (m, 2H), 1.69-1.36 (m, 1H); LCMS (Method B): t_{R} 2.99 min, 100%, MS (ESI) 413.2 (M + H)⁺ |
| 00109 | 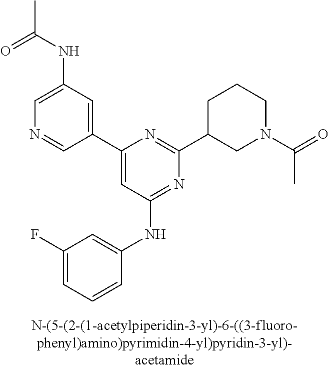 [SMILES: CC(=O)Nc1cncc(-c2cc(Nc3cccc(F)c3)nc(C3CCCN(C(C)=O)C3)n2)c1] | ¹H-NMR (400 MHz, DMSO-d6) mixture of rotamers δ 10.04 (d, J = 11.0 Hz, 1H), 9.32 (dd, J = 8.3, 2.1 Hz, 1H), 9.10 (t, J = 2.4 Hz, 1H), 8.84 (p, J = 4.5 Hz, 1H), 8.75 (dt, J = 4.6, 2.2 Hz, 1H), 7.95-7.82 (m, 1H), 7.47-7.33 (m, 2H), 7.27 (d, J = 4.8 Hz, 1H), 6.91-6.80 (m, 1H), 4.78-4.64 (m, 0.5H), 4.23 (dt, J = 13.1, 4.1 Hz, 0.5H), 4.15 (dd, J = 13.5, 3.9 Hz, 0.5H), 3.85 (d, J = 13.5 Hz, 0.5H), 3.52 (dd, J = 13.4, 10.2 Hz, 0.5H), 3.17-3.06 (m, 0.5H), 3.04-2.91 (m, 1H), 2.89- 2.73 (m, 4H), 2.24 (dt, J = 16.4, 5.3 Hz, 1H), 2.05 (s, 3H), 1.97- 1.72 (m, 2H), 1.68-1.40 (m, 1H); LCMS (Method B): t_{R} 2.90 min, 99%, MS (ESI) 449.2 (M + H)⁺ |
| 00110 | 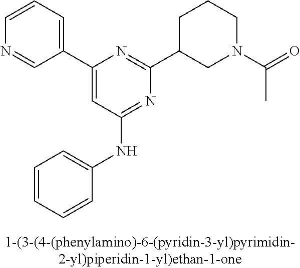 [SMILES: CC(=O)N1CCCC(c2nc(Nc3ccccc3)cc(-c3cccnc3)n2)C1] | ¹H-NMR (400 MHz, DMSO-d6) mixture of rotamers δ 9.75 (d, J = 7.7 Hz, 1H), 9.25-9.10 (m, 1H), 8.70 (d, J = 4.7 Hz, 1H), 8.50-8.28 (m, 1H), 7.75 (d, J = 7.9 Hz, 2H), 7.61-7.49 (m, 1H), 7.40-7.30 (m, 2H), 7.15 (d, J = 3.4 Hz, 1H), 7.04 (t, J = 7.4 Hz, 1H), 4.81-4.67 (m, 0.5H), 4.26-4.18 (m, 0.5H), 4.15- 4.05 (m, 0.5H), 3.90-3.80 (m, 0.5H), 3.56-3.46 (m, 0.5H), 3.16- 3.02 (m, 0.5H), 2.99-2.71 (m, 2H), 2.27-2.16 (m, 1H), 2.04 (d, J = 2.9 Hz, 3H), 1.95-1.71 (m, 2H), 1.66- 1.39 (m, 1H); LCMS (Method B): t_{R} 2.84 min, 100%, MS (ESI) 374.2 (M + H)⁺ |
| 00111 | 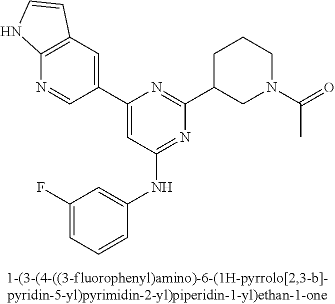 [SMILES: CC(=O)N1CCCC(c2nc(Nc3cccc(F)c3)cc(-c3cnc4[nH]ccc4c3)n2)C1] | ¹H-NMR (400 MHz, DMSO-d6) mixture of rotamers δ 11.89 (s, 1H), 9.89 (d, J = 10.6 Hz, 1H), 8.91 (s, 1H), 8.62 (s, 1H), 7.90 (t, J = 12.5 Hz, 1H), 7.56 (d, J = 3.4 Hz, 1H), 7.49-7.30 (m, 2H), 7.18 (d, J = 4.0 Hz, 1H), 6.83 (t, J = 8.4 Hz, 1H), 6.61 (s, 1H), 4.79 (d, J = 12.8 Hz, 0.5H), 4.21 (dd, J = 32.1, 13.5 Hz, 1H), 3.87 (d, J = 13.5 Hz, 0.5H), 3.55 (d, J = 10.2 Hz, 0.5H), 3.13-3.06 (m, 0.5H), 2.99-2.74 (m, 2H), 2.24 (s, 1H), 2.06 (s, 3H), 1.94-1.75 (m, 2H), 1.67-1.43 (m, 1H); LCMS (Method B): t_{R} 3.18 min, 99%, MS (ESI) 431.2 (M + H)⁺ |
| 00112 | 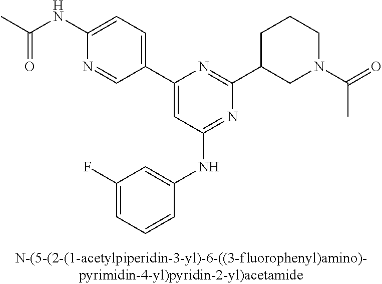 [SMILES: CC(=O)Nc1ccc(-c2cc(Nc3cccc(F)c3)nc(C3CCCN(C(C)=O)C3)n2)cn1] | ¹H-NMR (400 MHz, DMSO-d6) mixture of rotamers δ 10.77 (d, J = 3.0 Hz, 1H), 9.98 (d, J = 12.4 Hz, 1H), 9.01-8.95 (m, 1H), 8.41-8.33 (m, 1H), 8.23 (d, J = 8.8 Hz, 1H), 7.94-7.83 (m, 1H), 7.45-7.32 (m, 2H), 7.13 (d, J = 5.7 Hz, 1H), 6.83 (t, J = 8.4 Hz, 1H), 4.83-4.66 (m, 0.5H), 4.23 (d, J = 13.0 Hz, 0.5H), 4.18-4.09 (m, 0.5H), 3.86 (d, J = 13.4 Hz, 0.5H), 3.50 (dd, J = 13.4, 10.1 Hz, 0.5H), 3.08 (td, J = 13.5, 12.9, 2.7 Hz, 0.5H), 2.99-2.71 (m, 2H), 2.23 (d, J = 12.3 Hz, 1H), 2.14 (s, 3H), 2.05 (s, 3H), 1.93- 1.72 (m, 2H), 1.66-1.36 (m, 1H); LCMS (Method B): t_{R} 3.27 min, 99%, MS (ESI) 449.2 (M + H)⁺ |
| 00113 | 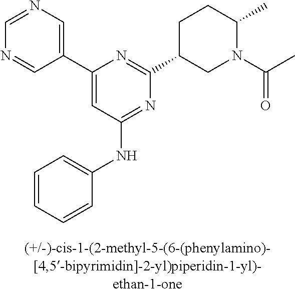 [SMILES: CC(=O)N1C[C@H](c2nc(Nc3ccccc3)cc(-c3cncnc3)n2)CC[C@@H]1C] | ¹H NMR (400 MHz, DMSO-d6) mixture of rotamers δ 9.88 (d, J = 7.6 Hz, 1H), 9.38 (d, J = 8.8 Hz, 2H), 9.32 (d, J = 2.1 Hz, 1H), 7.76 (dd, J = 8.3, 2.8 Hz, 2H), 7.36 (td, J = 8.8, 8.0, 2.0 Hz, 2H), 7.21 (d, J = 4.2 Hz, 1H), 7.06 (t, J = 7.3 Hz, 1H), 4.91-4.76 (m, 0.5H), 4.71 (dd, J = 13.2, 4.3 Hz, 0.5H), 4.21 (t, J = 6.4 Hz, 0.5H), 4.04 (dd, J = 14.0, 4.2 Hz, 0.5H), 3.45 (dd, J = 13.7, 11.8 Hz, 0.5H), 2.96-2.81 (m, 1H), 2.77-2.65 (m, 0.5H), 2.10-1.89 (m, 5H), 1.88-1.61 (m, 2H), 1.20 (dd, J = 52.1, 6.9 Hz, 3H); LCMS (Method D): t_{R} 3.37 min, 100%, MS (ESI) 389.2 (M + H)⁺ |
| 00114 | 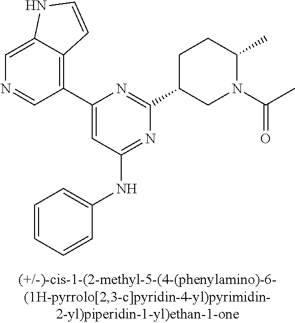 [SMILES: CC(=O)N1C[C@H](c2nc(Nc3ccccc3)cc(-c3cncc4[nH]ccc34)n2)CC[C@@H]1C] | ¹H NMR (400 MHz, DMSO-d6) mixture of rotamers δ 11.90 (s, 1H), 9.74-9.66 (m, 1H), 8.85 (s, 1H), 8.75-8.69 (m, 1H), 7.84-7.74 (m, 3H), 7.41-7.31 (m, 2H), 7.30- 7.24 (m, 1H), 7.09-6.99 (m, 2H), 4.88-4.73 (m, 1H), 4.27-4.17 (m, 0.5H), 4.14-4.05 (m, 0.5H), 3.52- 3.42 (m, 0.5H), 2.98-2.86 (m, 1H), 2.79-2.68 (m, 0.5H), 2.17-1.93 (m, 5H), 1.92-1.62 (m, 2H), 1.28 (d, J = 6.9 Hz, 1.5H), 1.15 (d, J = 7.0 Hz, 1.5H); LCMS (Method D): t_{R} 3.47 min, 97%, MS (ESI) 427.2 (M + H)⁺ |
| 00115 | 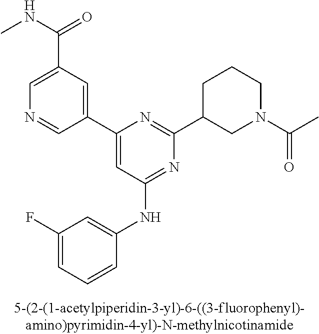 [SMILES: CNC(=O)c1cncc(-c2cc(Nc3cccc(F)c3)nc(C3CCCN(C(C)=O)C3)n2)c1] | ¹H NMR (400 MHz, chloroform- d) mixture of rotamers δ 10.03 (d, J = 10.8 Hz, 1H), 9.32 (dd, J = 8.3, 2.2 Hz, 1H), 9.10 (t, J = 2.4 Hz, 1H), 8.96-8.81 (m, 1H), 8.75 (dt, J = 4.7, 2.1 Hz, 1H), 7.97- 7.76 (m, 1H), 7.49-7.33 (m, 2H), 7.26 (d, J = 4.8 Hz, 1H), 6.85 (t, J = 8.5 Hz, 1H), 4.70 (d, J = 12.7 Hz, 0.5H), 4.31-4.07 (m, 1H), 3.85 (d, J = 13.2 Hz, 0.5H), 3.52 (dd, J = 13.4, 10.1 Hz, 0.5H), 3.11 (t, J = 12.8 Hz, 0.5H), 3.04- 2.89 (m, 1H), 2.88-2.72 (m, 4H), 2.22 (s, 1H), 2.04 (s, 3H), 1.96- 1.69 (m, 2H), 1.67-1.37 (m, 1H); LCMS (Method B): t_{R} 3.20 min, 99%, MS (ESI) 449.2 (M + H)⁺ |
| 00116 | 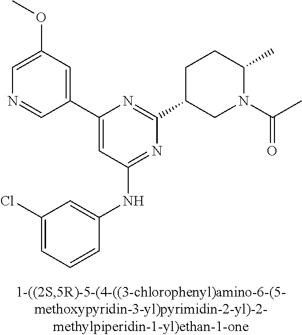 [SMILES: COc1cncc(-c2cc(Nc3cccc(Cl)c3)nc([C@@H]3CC[C@H](C)N(C(C)=O)C3)n2)c1] | ¹H NMR (400 MHz, DMSO-d6) mixture of rotamers δ 10.10-9.89 (m, 1H), 8.84-8.76 (m, 1H), 8.46- 8.39 (m, 1H), 8.28-8.21 (m, 1H), 7.94-7.88 (m, 1H), 7.55-7.48 (m, 1H), 7.41-7.33 (m, 1H), 7.20-7.15 (m, 1H), 7.12-7.04 (m, 1H), 4.89- 4.78 (m, 0.5H), 4.74-4.64 (m, 0.5H), 4.27-4.16 (m, 0.5H), 4.10- 4.01 (m, 0.5H), 3.97-3.88 (m, 3H), 3.51-3.40 (m, 0.5H), 2.97-2.84 (m, 1H), 2.81-2.70 (m, 0.5H), 2.11- 1.61 (m, 7H), 1.30 (d, J = 6.7 Hz, 1.5H), 1.16 (d, J = 7.1 Hz, 1.5H); LCMS (Method B): t_{R} 3.65 min, 99%, MS (ESI) 452.1 (M + H)⁺ |

### Example 2: Synthesis of 1-(3-(4-(pyridin-3-yl)-6-(p-tolylamino)pyrimidin-2-yl)piperidin-1-yl)propan-1-one (00117)

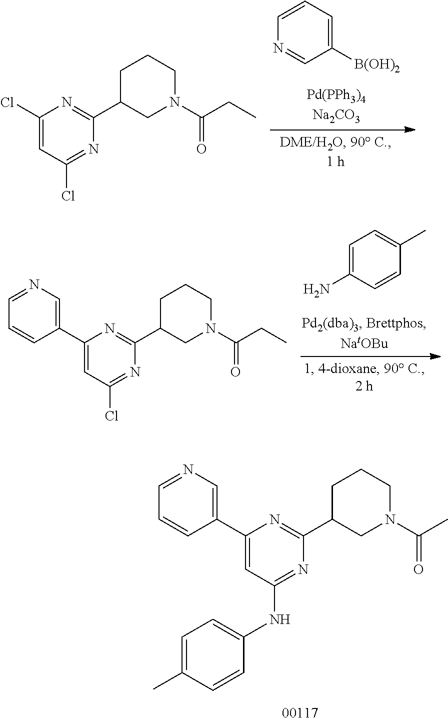

Under argon atmosphere, a microwave vial was charged with 1-(3-(4,6-dichloropyrimidin-2-yl)piperidin-1-yl)propan-1-one (1.1 g, 3.44 mmol), pyridine-3-boronic acid (0.40 g, 3.26 mmol), sodium carbonate (0.73 g, 6.87 mmol) and tetrakis(triphenylphosphine)palladium(0) (0.20 g, 0.17 mmol) in 1,2-dimethoxyethane (30 mL) and water (10 mL). The mixture was heated in a microwave to 90° C. for 1 hour. The mixture was poured into water and extracted with ethyl acetate twice. The combined organic layers were washed with brine, dried with sodium sulfate and concentrated in vacuo to afford an orange gum. The crude product was purified with silica column chromatography (1% to 10% methanol in dichloromethane) and lyophilized to afford 1-(3-(4-chloro-6-(pyridin-3-yl)pyrimidin-2-yl)piperidin-1-yl)propan-1-one (620 mg, 51%) as a brown solid. ¹H-NMR (400 MHz, DMSO-d6) mixture of rotamers δ 9.42 (s, 1H), 8.77 (d, J=4.8 Hz, 1H), 8.61 (dt, J=8.1, 2.1 Hz, 1H), 8.30 (d, J=3.8 Hz, 1H), 7.65-7.57 (m, 1H), 4.43 (dd, J=200.7, 10.8 Hz, 1H), 3.98 (dd, J=90.2, 13.9 Hz, 1H), 3.61-3.48 (m, 0.5H), 3.17-3.02 (m, 1H), 2.92 (d, J=9.7 Hz, 1H), 2.89-2.81 (m, 0.5H), 2.41-2.31 (m, 2H), 2.25-2.11 (m, 1H), 2.00-1.65 (m, 2H), 1.62-1.37 (m, 1H), 1.06-0.91 (m, 3H); LCMS (Method C): t_{R} 1.87 min, 100%, MS (ESI) 331.1 (M+H)⁺. Under argon atmosphere, Pd₂(dba)₃ (1.73 mg, 1.89 μmol; CAS Number 51364-51-3) and BrettPhos (2.03 mg, 3.78 μmol; CAS number 1070663-78-3) were dissolved in degassed 1,4-dioxane (1 mL) and heated to 90° C. for 2 minutes. The premixed catalyst was added to a mixture of 1-(3-(4-chloro-6-(pyridin-3-yl)pyrimidin-2-yl)piperidin-1-yl)propan-1-one (25 mg, 0.08 mmol), p-toluidine (8.91 mg, 0.08 mmol) and sodium tert-butoxide (8.72 mg, 0.09 mmol) in degassed 1,4-dioxane (1 mL). The mixture was heated at 90° C. for 2 hours, concentrated in vacuo and purified with reverse phase chromatography (Method B) to afford 1-(3-(4-(pyridin-3-yl)-6-(p-tolylamino)pyrimidin-2-yl)piperidin-1-yl)propan-1-one (11 mg, 34%) as a light yellow solid. ¹H-NMR (400 MHz, DMSO-d6) mixture of rotamers δ 9.64 (d, J=7.7 Hz, 1H), 9.18 (d, J=2.7 Hz, 1H), 8.69 (d, J=4.7 Hz, 1H), 8.36 (dt, J=8.0, 2.0 Hz, 1H), 7.58 (dd, J=24.4, 7.2 Hz, 3H), 7.17 (d, J=8.2 Hz, 2H), 7.10 (d, J=2.1 Hz, 1H), 4.75 (d, J=12.7 Hz, 0.5H), 4.24 (d, J=12.8 Hz, 0.5H), 4.14 (d, J=12.4 Hz, 0.5H), 3.89 (d, J=13.4 Hz, 0.5H), 3.46 (dd, J=13.4, 10.1 Hz, 0.5H), 3.04 (t, J=12.7 Hz, 0.5H), 2.95-2.63 (m, 2H), 2.42-2.32 (m, 2H), 2.28 (s, 3H), 2.20 (d, J=11.2 Hz, 1H), 1.97-1.69 (m, 2H), 1.64-1.37 (m, 1H), 1.07-0.92 (m, 3H); LCMS (Method D): t_{R} 3.42 min, 100%, MS (ESI) 402.2 (M+H)⁺.

The following compounds were prepared using procedures analogous to Example 2:

| Compound | | |
|---|---|---|
| # | Structure and compound name | Analytical data |
| 00118 | 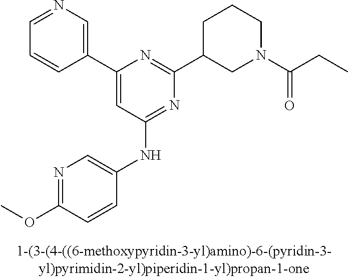 [SMILES: CCC(=O)N1CCCC(c2nc(Nc3ccc(OC)nc3)cc(-c3cccnc3)n2)C1] | ¹H-NMR (400 MHz, DMSO-d6) mixture of rotamers δ 9.69 (d, J = 5.1 Hz, 1H), 9.19 (s, 1H), 8.70 (d, J = 4.6 Hz, 1H), 8.52 (d, J = 12.1 Hz, 1H), 8.37 (d, J = 8.0, 1.9 Hz, 1H), 8.02 (t, J = 7.5 Hz, 1H), 7.68-7.50 (m, 3H), 7.07 (s, 1H), 6.86 (d, J = 8.8 Hz, 1H), 4.72 (d, J = 12.4 Hz, 0.5H), 4.17 (dd, J = 47.3, 13.9 Hz, 1H), 3.90 (m, 0.5H), 3.85 (s, 3H), 3.45 (dd, J = 13.4, 10.2 Hz, 0.5H), 3.04 (t, J = 12.7 Hz, 0.5H), 2.94-2.65 (m, 2H), 2.41-2.31 (m, 2H), 2.18 (d, J = 11.4 Hz, 1H), 1.94-1.69 (m, 2H), 1.64-1.37 (m, 1H), 1.04-0.92 (m, 3H); LCMS (Method D): t_{R} |
| | | 3.11 min, 100%, MS (ESI) 419.2 |
| | | (M + H)⁺ |
| 00119 | 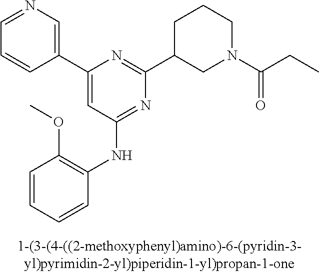 [SMILES: CCC(=O)N1CCCC(c2nc(Nc3ccccc3OC)cc(-c3cccnc3)n2)C1] | ¹H-NMR (400 MHz, DMSO-d6) mixture of rotamers δ 9.19 (d, J = 2.3 Hz, 1H), 8.89 (d, J = 10.8 Hz, 1H), 8.69 (d, J = 4.8 Hz, 1H), 8.36 (dt, J = 8.1, 2.0 Hz, 1H), 8.18 (d, J = 7.9 Hz, 1H), 7.56 (td, J = 6.5, 5.8, 2.8 Hz, 1H), 7.37 (s, 1H), 7.10 (d, J = 4.1 Hz, 2H), 6.98 (dt, J = 8.5, 4.3 Hz, 1H), 4.74 (d, J = 12.8 Hz, 0.5H), 4.23 (d, J = 12.9 Hz, 0.5H), 4.13 (d, J = 14.1 Hz, 0.5H), 3.88 (s, 3.5H), 3.45 (dd, J = 13.4, 10.2 Hz, 0.5H), 3.10-2.98 (m, 0.5H), 2.94-2.66 (m, 2H), 2.43-2.29 (m, 2H), 2.23-2.13 (m, 1H), 1.96- 1.69 (m, 2H), 1.62-1.36 (m, 1H), |
| | | 1.05-0.93 (m, 3H) ); LCMS |
| | | (Method D): t_{R} 3.34 min, 100%, |
| | | MS (ESI) 418.2 (M + H)⁺ |
| 00120 | 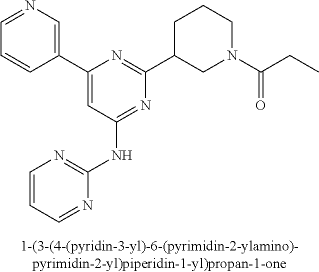 [SMILES: CCC(=O)N1CCCC(c2nc(Nc3ncccn3)cc(-c3cccnc3)n2)C1] | ¹H-NMR (400 MHz, DMSO-d6) mixture of rotamers δ 10.56 (d, J = 6.5 Hz, 1H), 9.28 (s, 1H), 8.78-8.67 (m, 4H), 8.46 (dt, J = 8.1, 2.0 Hz, 1H), 7.60 (dd, J = 7.8, 4.7 Hz, 1H), 7.13 (t, J = 4.8 Hz, 1H), 4.73 (d, J = 12.6 Hz, 0.5H), 4.25 (d, J = 13.1 Hz, 0.5H), 4.13 (d, J = 13.5 Hz, 0.5H), 3.90 (d, J = 13.3 Hz, 0.5H), 3.60-3.49 (m, 0.5H), 3.06 (t, J = 12.8 Hz, 0.5H), 2.98- 2.86 (m, 1H), 2.85-2.72 (m, 1H), 2.44-2.30 (m, 2H), 2.25-2.13 (m, 1H), 1.99-1.72 (m, 2H), 1.60-1.40 (m, 1H), 1.06-0.94 (m, 3H) ); LCMS (Method D): t_{R} 2.92 min, |
| | | 100%, MS (ESI) 390.2 (M + H)⁺ |
| 00121 | 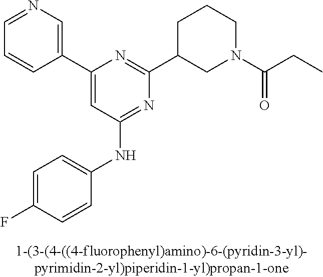 [SMILES: CCC(=O)N1CCCC(c2nc(Nc3ccc(F)cc3)cc(-c3cccnc3)n2)C1] | ¹H-NMR (400 MHz, DMSO-d6) mixture of rotamers δ 9.77 (d, J = 6.8 Hz, 1H), 9.19 (t, J = 2.3 Hz, 1H), 8.70 (d, J = 4.7 Hz, 1H), 8.37 (dt, J = 8.0, 2.0 Hz, 1H), 7.75 (dd, J = 8.9, 5.0 Hz, 2H), 7.56 (dd, J = 8.0, 4.8 Hz, 1H), 7.20 (t, J = 8.9 Hz, 2H), 7.10 (s, 1H), 4.73 (d, J = 12.7 Hz, 0.5H), 4.23 (d, J = 12.9 Hz, 0.5H), 4.12 (d, J = 13.1 Hz, 0.5H), 3.89 (d, J = 13.6 Hz, 0.5H), 3.47 (dd, J = 13.5, 10.2 Hz, 0.5H), 3.06 (t, J = 12.7 Hz, 0.5H), 2.92-2.65 (m, 2H), 2.41-2.28 (m, 2H), 2.19 (d, J = 12.8 Hz, 1H), 1.97-1.67 (m, 2H), 1.64-1.36 (m, 1H), 1.08- |
| | | 0.91 (m, 3H) ); LCMS (Method |
| | | D): t_{R} 3.31 min, 100%, MS (ESI) |
| | | 406.1 (M + H)⁺ |
| 00122 | 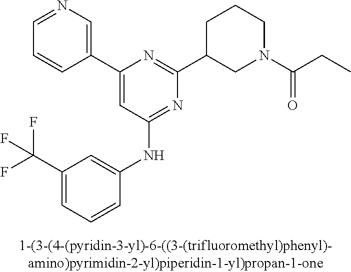 [SMILES: CCC(=O)N1CCCC(c2nc(Nc3cccc(C(F)(F)F)c3)cc(-c3cccnc3)n2)C1] | ¹H-NMR (400 MHz, DMSO-d6) mixture of rotamers δ 10.15 (d, J = 8.7 Hz, 1H), 9.22 (s, 1H), 8.72 (d, J = 4.7 Hz, 1H), 8.51 (d, J = 15.0 Hz, 1H), 8.40 (d, J = 8.0 Hz, 1H), 7.85 (dd, J = 18.4, 8.2 Hz, 1H), 7.59 (t, J = 7.6 Hz, 2H), 7.37 (d, J = 7.7 Hz, 1H), 7.19 (d, J = 4.4 Hz, 1H), 4.76 (d, J = 12.5 Hz, 0.5H), 4.29-4.11 (m, 1H), 3.91 (d, J = 13.8 Hz, 0.5H), 3.49 (dd, J = 13.5, 10.2 Hz, 0.5H), 3.08-2.67 (m, 2.5H), 2.40-2.31 (m, 2H), 2.29-2.17 (m, 1H), 2.03-1.69 (m, 2H), 1.64-1.39 (m, 1H), 1.06-0.92 (m, 3H); LCMS (Method D): t_{R} 3.58 min, 100%, |
| | | MS (ESI) 456.2 (M +H)⁺ |
| 00123 | 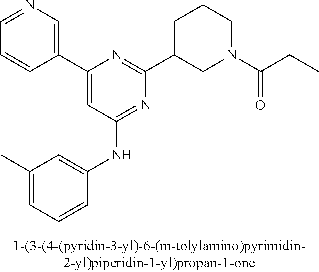 [SMILES: CCC(=O)N1CCCC(c2nc(Nc3cccc(C)c3)cc(-c3cccnc3)n2)C1] | ¹H-NMR (400 MHz, DMSO-d6) mixture of rotamers δ 9.70 (d, J = 8.5 Hz, 1H), 9.19 (t, J = 2.8 Hz, 1H), 8.70 (d, J = 4.7 Hz, 1H), 8.37 (dt, J = 8.0, 2.0 Hz, 1H), 7.62 (s, 1H), 7.59-7.50 (m, 2H), 7.24 (t, J = 7.8 Hz, 1H), 7.14 (d, J = 3.0 Hz, 1H), 6.86 (d, J = 7.6 Hz, 1H), 4.79 (d, J = 12.6 Hz, 0.5H), 4.24 (d, J = 12.9 Hz, 0.5H), 4.20-4.12 (m, 0.5H), 3.90 (d, J = 13.5 Hz, 0.5H), 3.47 (dd, J = 13.4, 10.1 Hz, 0.5H), 3.04 (t, J = 12.7 Hz, 0.5H), 2.97-2.68 (m, 2H), 2.43-2.34 (m, 2H), 2.33 (s, 3H), 2.22 (d, J = 12.8 Hz, 1H), 1.98-1.71 (m, 2H), 1.66-1.40 (m, |
| | | 1H), 1.07-0.93 (m, 3H); LCMS |
| | | (Method D): t_{R} 3.41 min, 100%, |
| | | MS (ESI) 402.2 (M + H)⁺ |
| 00124 | 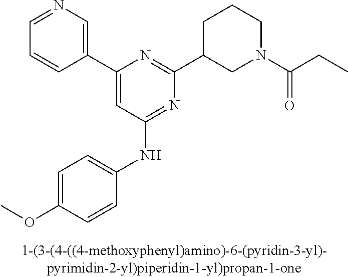 [SMILES: CCC(=O)N1CCCC(c2nc(Nc3ccc(OC)cc3)cc(-c3cccnc3)n2)C1] | ¹H-NMR (400 MHz, DMSO-d6) mixture of rotamers δ 9.57 (d, J = 6.8 Hz, 1H), 9.18 (d, J = 2.9 Hz, 1H), 8.69 (d, J = 4.7 Hz, 1H), 8.35 (dt, J = 8.0, 2.0 Hz, 1H), 7.62 (d, J = 8.4 Hz, 2H), 7.55 (dd, J = 7.9, 5.1 Hz, 1H), 7.04 (d, J = 1.7 Hz, 1H), 6.99-6.91 (m, 2H), 4.73 (d, J = 12.8 Hz, 0.5H), 4.24 (d, J = 12.9 Hz, 0.5H), 4.12 (d, J = 13.5 Hz, 0.5H), 3.89 (d, J = 13.6 Hz, 0.5H), 3.75 (s, 3H), 3.46 (dd, J = 13.5, 10.2 Hz, 0.5H), 3.04 (t, J = 12.8 Hz, 0.5H), 2.91-2.65 (m, 2H), 2.43- 2.31 (m, 2H), 2.25-2.13 (m, 1H), 1.81 (td, J = 27.3, 26.4, 12.6 Hz, |
| | | 2H), 1.65-1.36 (m, 1H), 0.99 (dt, |
| | | J = 13.1, 7.4 Hz, 3H); LCMS |
| | | (Method D): t_{R} 3.21 min, 100%, |
| | | MS (ESI) 418.2 (M + H)⁺ |
| 00125 | 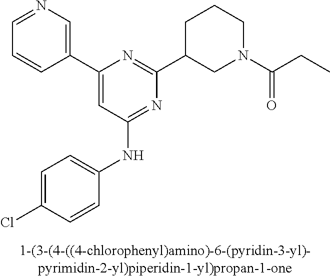 [SMILES: CCC(=O)N1CCCC(c2nc(Nc3ccc(Cl)cc3)cc(-c3cccnc3)n2)C1] | ¹H-NMR (400 MHz, DMSO-d6) mixture of rotamers δ 9.89 (d, J = 7.3 Hz, 1H), 9.20 (d, J = 2.6 Hz, 1H), 8.70 (d, J = 4.8 Hz, 1H), 8.38 (dt, J = 8.0, 2.0 Hz, 1H), 7.79 (d, 2H), 7.57 (dd, J = 7.9, 4.9 Hz, 1H), 7.41 (d, J = 8.6 Hz, 2H), 7.15 (d, J = 2.1 Hz, 1H), 4.73 (d, J = 12.6 Hz, 0.5H), 4.24 (d, J = 13.0 Hz, 0.5H), 4.13 (d, J = 13.4 Hz, 0.5H), 3.89 (d, J = 13.6 Hz, 0.5H), 3.48 (dd, J = 13.5, 10.2 Hz, 0.5H), 3.07 (t, J = 12.6 Hz, 0.5H), 2.97-2.69 (m, 2H), 2.43-2.30 (m, 2H), 2.20 (d, J = 12.6 Hz, 1H), 1.98-1.69 (m, 2H), 1.65-1.38 (m, 1H), 1.08- |
| | | 0.92 (m, 3H); LCMS (Method |
| | | D): t_{R} 3.50 min, 100%, MS (ESI) |
| | | 422.2 (M +H)⁺ |
| 00126 | 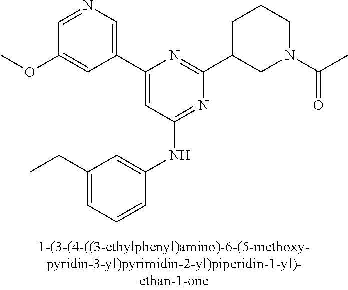 [SMILES: CCc1cccc(Nc2cc(-c3cncc(OC)c3)nc(C3CCCN(C(C)=O)C3)n2)c1] | ¹H-NMR (400 MHz, DMSO-d6) mixture of rotamers δ 9.70 (d, J = 8.7 Hz, 1H), 8.78 (d, J = 6.4 Hz, 1H), 8.42 (d, J = 3.0 Hz, 1H), 7.89 (d, J = 2.6 Hz, 1H), 7.70 (s, 1H), 7.50 (d, J = 8.3 Hz, 1H), 7.25 (t, J = 7.8 Hz, 1H), 7.14 (d, J = 4.4 Hz, 1H), 6.89 (d, J = 7.5 Hz, 1H), 4.74 (s, 0.5H), 4.26- 4.05 (m, 1H), 3.93 (s, 3H), 3.90- 3.80 (m, 0.5H), 3.61-3.47 (m, 1H), 3.15-3.00 (m, 0.5H), 3.01- 2.72 (m, 1.5H), 2.66-2.57 (m, 2H), 2.27-2.15 (m, 1H), 2.03 (d, J = 2.9 Hz, 3H), 1.97-1.70 (m, 2H), 1.65-1.41 (m, 1H), 1.22 (t, J = 7.6 Hz, 3H); LGMS (Method D): t_{R} 3.52 min, 98%, MS (ESI) |
| | | 432.2 (M + H)⁺ |
| 00127 | 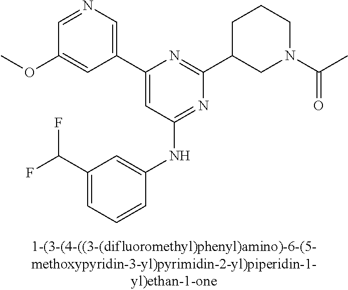 [SMILES: COc1cncc(-c2cc(Nc3cccc(C(F)F)c3)nc(C3CCCN(C(C)=O)C3)n2)c1] | ¹H-NMR (400 MHz, DMSO-d6) mixture of rotamers δ 10.02 (d, J = 8.8 Hz, 1H), 8.80 (d, J = 6.8 Hz, 1H), 8.43 (d, J = 2.9 Hz, 1H), 8.24 (d, J = 6.3 Hz, 1H), 7.92 (t, J = 2.4 Hz, 1H), 7.79 (t, J = 9.2 Hz, 1H), 7.49 (t, J = 7.9 Hz, 1H), 7.25-7.19 (m, 2H), 7.01 (t, J = 55.4 Hz 1H), 4.74-4.66 (m, 0.5H), 4.19 (d, J = 13.0 Hz, 0.5H), 4.11 (d, J = 12.9 Hz, 0.5H), 3.94 (s, 3H), 3.85 (d, J = 13.8 Hz, 0.5H), 3.65-3.52 (m, 0.5H), 3.09 (t, J = 12.4 Hz, 0.5H), 3.03-2.88 (m, 1H), 2.87- 2.74 (m, 1H), 2.22 (d, J = 12.8 Hz, 1H), 2.04 (d, J = 5.4 Hz, 3H), 1.96-1.71 (m, 2H), 1.69- |
| | | 1.38 (m, 1H); LCMS (Method |
| | | D): t_{R} 3.33 min, 100%, MS (ESI) |
| | | 454.1 (M + H)⁺ |
| 00128 | 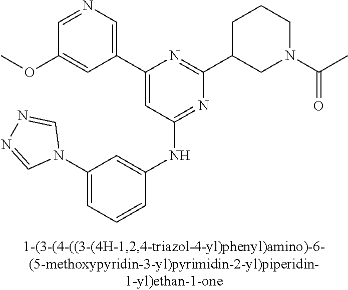 [SMILES: COc1cncc(-c2cc(Nc3cccc(-n4cnnc4)c3)nc(C3CCCN(C(C)=O)C3)n2)c1] | ¹H-NMR (400 MHz, DMSO-d6) mixture of rotamers δ 10.11 (d, J = 12.4 Hz, 1H), 9.09 (s, 2H), 8.82 (d, J = 5.3 Hz, 1H), 8.44 (t, J = 2.6 Hz, 1H), 8.30 (s, 1H), 7.93 (t, J = 2.3 Hz, 1H), 7.65 (d, J = 8.3 Hz, 1H), 7.55 (t, J = 8.1 Hz, 1H), 7.38-7.30 (m, 1H), 7.25 (d, J = 4.8 Hz, 1H), 4.67 (d, J = 12.8 Hz, 0.5H), 4.19 (d, J = 12.8 Hz, 0.5H), 4.11 (d, J = 13.7 Hz, 0.5H), 3.94 (s, 3H), 3.83 (d, J = 13.6 Hz, 0.5H), 3.57-3.48 (m, 0.5H), 3.09 (t, J = 12.4 Hz, 1H), 2.98 (t, J = 11.7 Hz, 1H), 2.89- 2.71 (m, 1H), 2.21 (d, J = 12.8 Hz, 1H), 2.11-1.70 (m, 5H), 1.65-1.38 (m, 1H); LCMS |
| | | (Method D): t_{R} 2.78 min, 100%, |
| | | MS (ESI) 471.2 (M + H)⁺ |

The following further compounds were prepared using procedures analogous to Example 2.

| Compound | | |
|---|---|---|
| # | Structure and compound name | Analytical data |
| 00129 | 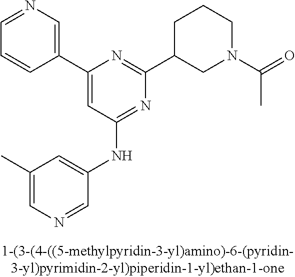 [SMILES: CC(=O)N1CCCC(c2nc(Nc3cncc(C)c3)cc(-c3cccnc3)n2)C1] | 1H-NMR (400 MHz, DMSO-d6) mixture of rotamers δ 9.92 (s, 1H), 9.25-9.17 (m, 1H), 8.71 (d, J = 4.5 Hz, 2H), 8.44-8.33 (m, 1H), 8.10 (d, J = 7.6 Hz, 2H), 7.61-7.52 (m, 1H), 7.19 (d, J = 5.8 Hz, 1H), 4.78 (dd, J = 11.9, 2.8 Hz, 0.5H), 4.21 (d, J = 12.9 Hz, 0.5H), 4.13 (dd, J = 13.4, 3.9 Hz, 0.5H), 3.88-3.81 (m, 0.5H), 3.51 (dd, J = 13.4, 10.1 Hz, 0.5H), 3.13-3.02 (m, 0.5H), 3.01-2.91 (m, 0.5H), 2.91-2.73 (m, 1.5H), 2.33 (d, J = 3.5 Hz, 3H), 2.23 (t, J = 11.6 Hz, 1H), 2.04 (d, J = 2.4 Hz, 3H), 1.96- 1.71 (m, 2H), 1.68-1.40 (m, |
| | | 1H); LCMS (Method D): tR |
| | | 3.03 min, 98%, MS (ESI) 389.2 |
| | | (M + H)⁺ |
| 00130 | 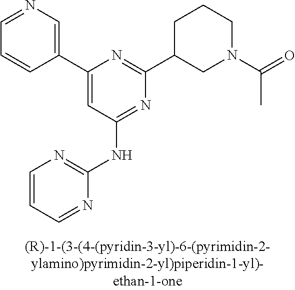 [SMILES: CC(=O)N1CCCC(c2nc(Nc3ncccn3)cc(-c3cccnc3)n2)C1] | 1H-NMR (400 MHz, DMSO-d6) mixture of rotamers δ 10.56 (s, 1H), 9.32-9.22 (m, 1H), 8.79- 8.65 (m, 4H), 8.51-8.40 (m, 1H), 7.65-7.53 (m, 1H), 7.16-7.06 (m, 1H), 4.80-4.67 (m, 0.5H), 4.28- 4.16 (m, 0.5H), 4.13-4.06 (m, 0.5H), 3.91-3.82 (m, 0.5H), 3.63-3.53 (m, 0.5H), 3.14-3.04 (m, 0.5H), 3.00-2.71 (m, 2H), 2.25-2.13 (m, 1H), 2.10-1.99 (m, 3H), 1.99-1.71 (m, 2H), 1.65-1.37 (m, 1H); LCMS (Method D): tR 2.98 min, 95%, MS (ESI) 376.2 (M + H)⁺ |
| 00131 | 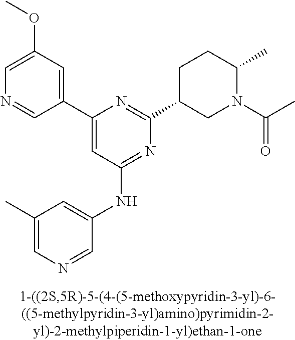 [SMILES: COc1cncc(-c2cc(Nc3cncc(C)c3)nc([C@@H]3CC[C@H](C)N(C(C)=O)C3)n2)c1] | ¹H NMR (400 MHz, DMSO-d6) mixture of rotamers δ 9.92 (s, 1H), 8.81 (dd, J = 6.6, 1.8 Hz, 1H), 8.69 (dd, J = 10.7, 2.5 Hz, 1H), 8.44 (t, J = 2.5 Hz, 1H), 8.18 (d, J = 16.3 Hz, 1H), 8.10- 8.08 (m, 1H), 7.94-7.89 (m, 1H), 7.20 (d, J = 4.6 Hz, 1H), 4.83 (S, 0.5H), 4.74 (dd, J = 13.2, 4.2 Hz, 0.5H), 4.22 (t, J = 6.5 Hz, 0.5H), 4.03 (dd, J = 13.8, 4.2 Hz, 0.5H), 3.93 (d, J = 1.0 Hz, 3H), 3.44 (dd, J = 13.5, 12.0 Hz, 0.5H), 2.96- 2.84 (m, 1H), 2.79-2.68 (m, 0.5H), 2.33 (d, J = 3.6 Hz, 3H), 2.10-1.97 (m, 5H), 1.90-1.79 (m, 0.5H), 1.74-1.64 (m, 1.5H), 1.27 (d, J = 6.8 Hz, 1.5H), 1.15 (d, J = 6.9 Hz, 1.5H); LCMS (Method D): t_{R} 3.27 min, 99%, |
| | | MS (ESI) 433.2 (M + H)⁺ |
| 00132 | 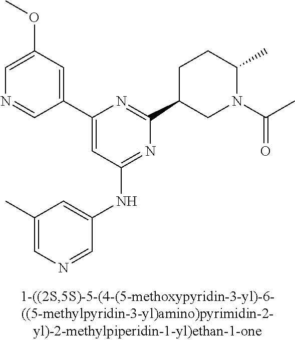 [SMILES: COc1cncc(-c2cc(Nc3cncc(C)c3)nc([C@H]3CC[C@H](C)N(C(C)=O)C3)n2)c1] | ¹H NMR (400 MHz, DMSO-d6) mixture of rotamers δ 9.88 (s, 1H), 8.79 (d, J = 1.7 Hz, 1H), 8.66 (d, J = 2.4 Hz, 1H), 8.42 (d, J = 2.9 Hz, 1H), 8.15-7.85 (m, 3H), 7.17 (s, 1H), 5.27 (s, 0.2H), 4.55 (d, J = 40.0 Hz, 1H), 4.14-4.03 (m, 0.2H), 3.94 (s, 3H), 3.57 (s, 0.5H), 3.27- 2.97 (m, 2H), 2.40 (d, J = 13.2 Hz, 1H), 2.32 (s, 3H), 2.14- 2.00 (m, 1H), 1.86 (s, 4H), 1.42 (d, J = 13.3 Hz, 1H), 1.28-1.09 (m, 3H); LCMS (Method D): t_{R} 3.33 min, 97%, MS (ESI) 433.2 (M + H)⁺ |

### Example 3: Synthesis of 1-(3-(4-((3-chlorophenyl)amino)-6-(pyridin-3-yl)pyrimidin-2-yl)piperidin-1-yl)propan-1-one (000133)

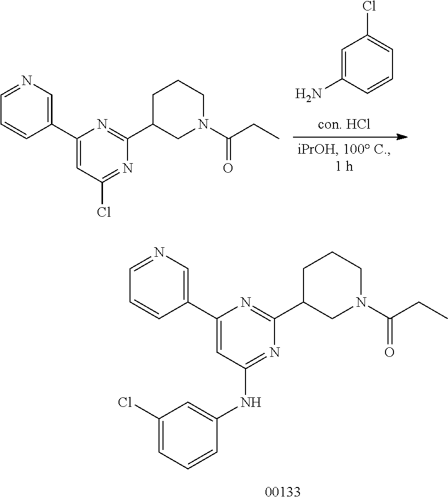 [SMILES: CCC(=O)N1CCCC(c2nc(Cl)cc(-c3cccnc3)n2)C1 ; Nc1cccc(Cl)c1 ; CCC(=O)N1CCCC(c2nc(Nc3cccc(Cl)c3)cc(-c3cccnc3)n2)C1]

To a solution of 1-(3-(4-chloro-6-(pyridin-3-yl)pyrimidin-2-yl)piperidin-1-yl)propan-1-one (25 mg, 0.06 mmol) and 3-chloroaniline (11.57 mg, 0.09 mmol) in 2-propanol (2 mL) concentrated hydrochloric acid (0.03 mL, 0.36 mmol) was added and the resulting mixture was heated at 100° C. for 1 hour. The mixture was concentrated and purified with reverse phase chromatography (Method B) to afford 1-(3-(4-((3-chlorophenyl)amino)-6-(pyridin-3-yl)pyrimidin-2-yl)piperidin-1-yl)propan-1-one (6 mg, 23%) as a white solid. ¹H-NMR (400 MHz, DMSO-d6) mixture of rotamers δ 9.97 (d, J=11.0 Hz, 1H), 9.21 (s, 1H), 8.71 (d, J=4.9 Hz, 1H), 8.39 (d, J=8.0 Hz, 1H), 8.11 (d, J=38.5 Hz, 1H), 7.66-7.52 (m, 2H), 7.38 (t, J=8.1 Hz, 1H), 7.16 (d, J=3.8 Hz, 1H), 7.08 (d, J=8.0 Hz, 1H), 4.76 (d, J=12.2 Hz, 0.5H), 4.22 (dd, J=24.8, 13.3 Hz, 1H), 3.90 (d, J=13.8 Hz, 0.5H), 3.59-3.39 (m, 0.5H), 3.20-3.00 (m, 0.5H), 3.00-2.85 (m, 1H), 2.85-2.70 (m, 1H), 2.44-2.31 (m, 3H), 2.30-2.18 (m, 1H), 2.01-1.71 (m, 2H), 1.63-1.39 (m, 1H), 1.05-0.94 (m, 3H); LCMS (Method D: t_{R} 3.59 min, 100%, MS (ESI) 422.2/424.2 (M+H)⁺.

The following compounds were prepared using procedures analogous to Example 3:

| Compound | | |
|---|---|---|
| # | Structure and compound name | Analytical data |
| 00134 | 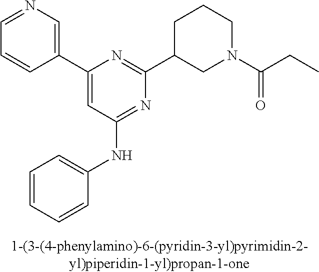 [SMILES: CCC(=O)N1CCCC(c2nc(Nc3ccccc3)cc(-c3cccnc3)n2)C1] | ¹H-NMR (400 MHz, DMSO-d6) mixture of rotamers δ 9.90 (s, 1H), 9.23 (s, 1H), 8.76 (S, 1H), 8.47 (s, 1H), 7.75 (d, J = 8.0 Hz, 2H), 7.68 (s, 1H), 7.43-7.29 (m, 2H), 7.18 (s, 1H), 7.06 (t, J = 7.5 Hz, 1H), 4.74 (d, J = 12.4 Hz, 1H), 4.25 (s, 0.5H), 4.16 (d, J = 13.8 Hz, 0.5H), 3.53-3.38 (m, 0.5H), 3.06 (t, J = 12.8 Hz, 0.5H), 2.97-2.83 (m, 1H), 2.83-2.70 (m, 1H), 2.42-2.30 (m, 2H), 2.20 (s, 1H), 1.92-1.70 (m, 2H), 1.63-1.39 (m, 1H), 1.09-0.88 (m, 3H); LCMS (Method B): t_{R} 2.87 min, 100%, MS (ESI) 388.2 (M + H)⁺ |
| 00135 | 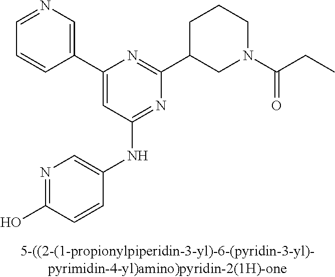 [SMILES: CCC(=O)N1CCCC(c2nc(Nc3ccc(O)nc3)cc(-c3cccnc3)n2)C1] | ¹H-NMR (400 MHz, DMSO-d6) mixture of rotamers δ 10.06 (d, J = 9.4 Hz, 1H), 7.76 (t, J = 14.2 Hz, 1H), 7.45-7.24 (m, 2H), 6.88 (t, J = 8.7 Hz, 1H), 6.71 (d, J = 3.4 Hz, 1H), 4.71 (d, J = 9.9 Hz, 0.5H), 4.21 (d, J = 13.0 Hz, 0.5H), 4.12-4.03 (m, 0.5H), 3.88 (d, J = 14.0 Hz, 0.5H), 3.40-3.29 (m, 1H), 3.06- 2.95 (m, 0.5H), 2.92-2.63 (m, 2H), 2.44-2.30 (m, 2H), 2.19- 2.08 (m, 1H), 1.87-1.64 (m, 2H), 1.61-1.34 (m, 1H), 1.05- 0.93 (m, 3H); LCMS (Method B): t_{R} 2.25 min, 100%, MS (ESI) 405.2 (M + H)⁺ |
| 00136 | 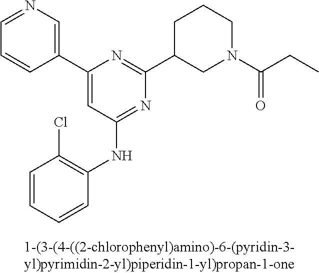 [SMILES: CCC(=O)N1CCCC(c2nc(Nc3ccccc3Cl)cc(-c3cccnc3)n2)C1] | ¹H-NMR (400 MHz, DMSO-d6) mixture of rotamers δ 9.30-9.16 (m, 2H), 8.70 (d, J = 4.7 Hz, 1H), 8.36 (dt, J = 7.8, 2.0 Hz, 1H), 8.01-7.93 (m, 1H), 7.59- 7.52 (m, 2H), 7.44-7.34 (m, 1H), 7.27 (s, 1H), 7.20 (t, J = 8.0 Hz, 1H), 4.73-4.63 (m, 0.5H), 4.18 (d, J = 12.9 Hz, 0.5H), 4.07 (d, J = 13.3 Hz, 0.5H), 3.86 (d, J = 13.5 Hz, 0.5H), 3.48-3.38 (m, 0.5H), 3.01 (t, J = 12.5 Hz, 0.5H), 2.87-2.65 (m, 2H), 2.38-2.28 (m, 2H), 2.21-2.08 (m, 1H), 1.94-1.65 (m, 2H), 1.59-1.36 (m, 1H), 1.05-0.91 (m, 3H); |
| | | LCMS (Method D): t_{R} 3.42 |
| | | min, 100%, MS (ESI) 422.2 |
| | | (M + H)⁺ |
| 00137 | 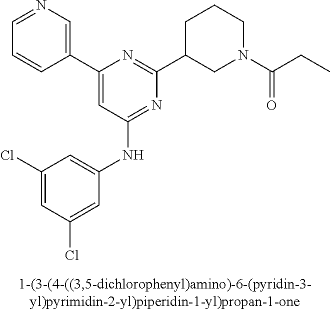 [SMILES: CCC(=O)N1CCCC(c2nc(Nc3cc(Cl)cc(Cl)c3)cc(-c3cccnc3)n2)C1] | ¹H-NMR (400 MHz, DMSO-d6) mixture of rotamers δ 10.19 (d, J = 10.6 Hz, 1H), 9.27-9.17 (m, 1H), 8.72 (d, J = 4.7 Hz, 1H), 8.41 (d, J = 8.0 Hz, 1H), 7.92 (t, J = 2.0 Hz, 2H), 7.63-7.53 (m, 1H), 7.27-7.15 (m, 2H), 4.76 (d, J = 12.5 Hz, 0.5H), 4.23 (t, J = 15.6 Hz, 1H), 3.90 (d, J = 13.4 Hz, 0.5H), 3.51-3.40 (m, 0.5H), 3.11-2.66 (m, 2.5H), 2.45-2.32 (m, 2H), 2.25 (s, 1H), 2.00-1.71 (m, 2H), 1.67-1.40 (m, 1H), 1.05-0.94 (m, 3H); LCMS (Method D): t_{R} 3.58 min, 100%, MS (ESI) 456.2/(M + H)⁺ |
| 00138 | 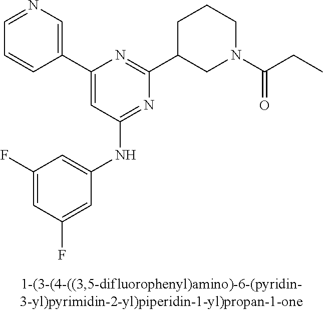 [SMILES: CCC(=O)N1CCCC(c2nc(Nc3cc(F)cc(F)c3)cc(-c3cccnc3)n2)C1] | ¹H-NMR (400 MHz, DMSO-d6) mixture of rotamers δ 10.22 (d, J = 11.9 Hz, 1H), 9.22 (t, J = 3.2 Hz, 1H), 8.72 (d, J = 4.7 Hz, 1H), 8.40 (d, J = 8.0 Hz, 1H), 7.63-7.49 (m, 3H), 7.20 (d, J = 4.3 Hz, 1H), 6.86 (t, J = 9.2 Hz, 1H), 4.77 (d, J = 12.2 Hz, 0.5H), 4.32-4.17 (m, 1H), 3.90 (d, J = 13.3 Hz, 0.5H), 3.43 (dd, J = 13.3, 10.4 Hz, 0.5H), 3.15-2.61 (m, 2.5H), 2.38 (dq, J = 15.4, 7.6 Hz, 2H), 2.29-2.13 (m, 1H), 1.98-1.68 (m, 2H), 1.67-1.35 (m, 1H), 1.12-0.93 (m, 3H).; LCMS (Method D): t_{R} 2.49 min, 100%, MS (ESI) 424.2 (M + H)⁺ |
| 00139 | 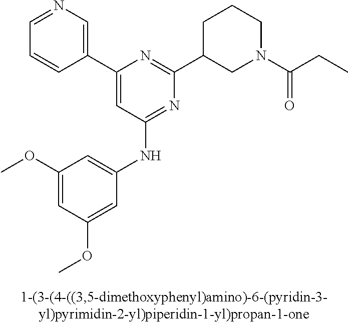 [SMILES: CCC(=O)N1CCCC(c2nc(Nc3cc(OC)cc(OC)c3)cc(-c3cccnc3)n2)C1] | ¹H-NMR (400 MHz, DMSO-d6) mixture of rotamers δ 9.75 (d, J = 6.7 Hz, 1H), 9.19 (d, J = 2.5 Hz, 1H), 8.70 (d, J = 4.7 Hz, 1H), 8.37 (dt, J = 8.1, 2.1 Hz, 1H), 7.57 (dd, J = 8.1, 4.9 Hz, 1H), 7.14 (s, 1H), 7.06 (s, 2H), 6.25-6.14 (m, 1H), 4.79 (d, J = 12.4 Hz, 0.5H), 4.28 (s, 0.5H), 4.17 (d, J = 14.6 Hz, 0.5H), 3.90 (d, J = 13.7 Hz, 1H), 3.76 (s, 6H), 3.51-3.39 (m, 0.5H), 3.09- 2.64 (m, 2.5H), 2.44-2.30 (m, 2H), 2.23 (d, J = 12.9 Hz, 1H), 2.00-1.70 (m, 2H), 1.64-1.39 (m, 1H), 1.07-0.92 (m, 3H); LCMS (Method D): t_{R} 3.29 min, 100%, MS (ESI) 448.2 (M + H)⁺ |

### Example 4: Synthesis of 1-(3-(4-((3-fluorophenyl)amino)-6-(pyridin-3-yl)pyrimidin-2-yl)piperidin-1-yl)ethan-1-one (00142)

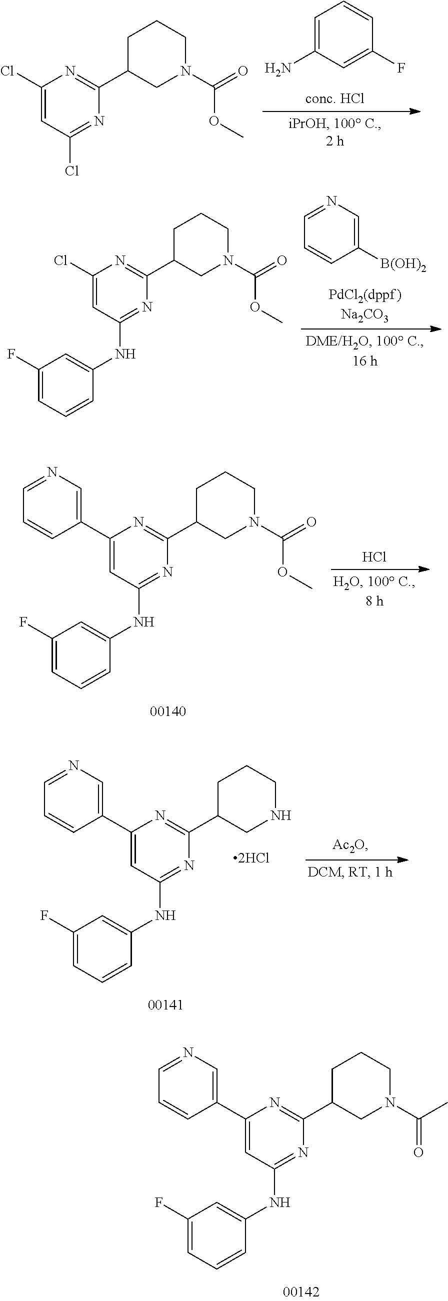

To a solution of methyl 3-(4,6-dichloropyrimidin-2-yl)piperidine-1-carboxylate (1 g, 3.45 mmol) and 3-fluoroaniline (0.46 g, 4.14 mmol) in 2-propanol (10 mL) was added concentrated hydrochloric acid (1.01 mL, 12.1 mmol) and the mixture was stirred at 100° C. for 2 hours. Heating was removed, additional 2-propanol (10 mL) was added and the mixture left to cool and crystallize. The crystals were filtered off and air dried to afford methyl 3-(4-chloro-6-((3-fluorophenyl)amino)pyrimidin-2-yl)piperidine-1-carboxylate (1.0 g, 82%) as a white solid. ¹H-NMR (400 MHz, DMSO-d6) δ 10.15 (s, 1H), 7.76 (d, J=11.9 Hz, 1H), 7.43-7.30 (m, 2H), 6.93-6.82 (m, 1H), 6.74 (s, 1H), 4.28 (s, 1H), 3.77 (p, J=6.1 Hz, 1H), 3.60 (s, 3H), 3.18-2.70 (m, 3H), 2.20-2.05 (m, 1H), 1.80-1.62 (m, 2H), 1.56-1.40 (m, 1H)); LCMS (Method A): t_{R} 2.11 min, 100%, MS (ESI) 365.1 (M+H)⁺. Under argon atmosphere, methyl 3-(4-chloro-6-((3-fluorophenyl)amino)pyrimidin-2-yl)piperidine-1-carboxylate (52 mg, 0.14 mmol), pyridin-3-ylboronic acid (35 mg, 0.29 mmol) and sodium carbonate (45 mg, 0.43 mmol) were dissolved in a mixture of water (2 mL) and 1,2-dimethoxyethane (8 mL). Next, PdCl₂(dppf) (5.2 mg, 7.13 μmol; CAS number 72287-26-4) was added and the reaction mixture was stirred at 100° C. for 16 hours. The mixture was neutralised with formic acid to ˜pH 7, filtered and concentrated in vacuo. The residue was purified by reversed phase chromatography (method A) to afford methyl 3-(4-((3-fluorophenyl)amino)-6-(pyridin-3-yl)pyrimidin-2-yl)piperidine-1-carboxylate (20 mg, 28%) as a yellow sticky solid. LCMS (Method A): t_{R} 1.90 min, 100%, MS (ESI) 408.1 (M+H)⁺. A solution of methyl 3-(4-((3-fluorophenyl)amino)-6-(pyridin-3-yl)pyrimidin-2-yl)piperidine-1-carboxylate (500 mg, 1.23 mmol) in 6M hydrochloric acid (50 mL, 300 mmol) was heated at 100° C. for 8 hours. The mixture was concentrated in vacuo to afford N-(3-fluorophenyl)-2-(piperidin-3-yl)-6-(pyridin-3-yl)pyrimidin-4-amine dihydrochloride (490 mg, 95%) as a slightly yellow solid. ¹H-NMR (400 MHz, DMSO-d6) δ 10.25 (s, 1H), 9.29 (d, J=2.3 Hz, 1H), 8.92-8.75 (m, 3H), 8.65-8.47 (m, 1H), 7.85-7.79 (m, 1H), 7.79-7.70 (m, 1H), 7.48-7.34 (m, 2H), 7.33-7.23 (m, 1H), 6.94-6.81 (m, 1H), 3.66 (d, J=9.1 Hz, 1H), 3.36-3.21 (m, 3H), 2.95 (s, 1H), 2.32-2.18 (m, 1H), 1.97-1.73 (m, 3H); LCMS (Method B): t_{R} 2.25 min, 100%, MS (ESI) 350.1 (M+H)⁺. To a solution of N-(3-fluorophenyl)-2-(piperidin-3-yl)-6-(pyridin-3-yl)pyrimidin-4-amine (10 mg, 0.03 mmol) in dichloromethane (2 mL) acetic anhydride (100 μl, 1.06 mmol) was added and the mixture was stirred at room temperature for 1 hour. The mixture was concentrated and purified by reversed phase chromatography (method A) to afford 1-(3-(4-((3-fluorophenyl)amino)-6-(pyridin-3-yl)pyrimidin-2-yl)piperidin-1-yl)ethan-1-one (9.4 mg, 83%) as a white solid. ¹H-NMR (400 MHz, DMSO-d6) mixture of rotamers δ 9.98 (d, J=9.6 Hz, 1H), 9.21 (dd, J=5.8, 2.3 Hz, 1H), 8.71 (d, J=4.1, 2.2 Hz, 1H), 8.42-8.36 (m, 1H), 7.94-7.84 (m, 1H), 7.61-7.54 (m, 1H), 7.46-7.35 (m, 2H), 7.17 (d, J=4.1 Hz, 1H), 6.85 (t, J=8.6 Hz, 1H), 4.75 (d, J=12.5 Hz, 0.5H), 4.19 (dd, J=31.9, 13.3 Hz, 1H), 3.86 (d, J=13.3 Hz, 0.5H), 3.51 (dd, J=13.5, 10.2 Hz, 0.5H), 3.14-3.03 (m, 0.5H), 3.02-2.93 (m, 0.5H), 2.88 (t, 0.5H), 2.83-2.75 (m, 1H), 2.29-2.19 (m, 1H), 2.04 (s, 3H), 1.95-1.71 (m, 2H); LCMS (Method B): t_{R} 2.90 min, 100%, MS (ESI) 392.2 (M+H)⁺.

The following compounds were prepared using procedures analogous to Example 4:

| Compound | | |
|---|---|---|
| # | Structure and compound name | Analytical data |
| 00143 | 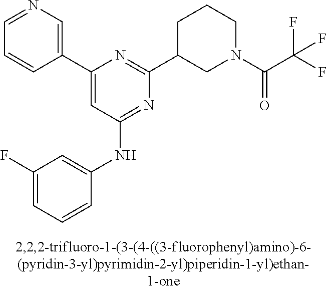 [SMILES: O=C(N1CCCC(c2nc(Nc3cccc(F)c3)cc(-c3cccnc3)n2)C1)C(F)(F)F] | ¹H-NMR (400 MHz, DMSO-d6) mixture of rotamers δ 10.03 (d, J = 10.6 Hz, 1H), 9.21 (d, J = 2.2 Hz, 1H), 8.71 (d, J = 4.8 Hz, 1H), 8.42-8.35 (m, 1H), 7.92- 7.78 (m, 1H), 7.63-7.53 (m, 1H), 7.48-7.31 (m, 2H), 7.20 (d, J = 3.4 Hz, 1H), 6.89-6.81 (m, 1H), 4.63 (d, J = 13.1 Hz, 0.5H), 4.45-4.29 (m, 1H), 3.93-3.81 (m, 0.5H), 3.58-3.48 (m, 0.5H), 3.42-3.36 (m, 1H), 3.06-2.89 (m, 1,5H), 2.45-2.22 (m, 1.5H), 1.99-1.84 (m, 2H), 1.73-1.56 (m, 1H); LCMS (Method D): t_{R} 3.81 min, 100%, MS (ESI) 446.1 (M + H)⁺. |
| 00144 | 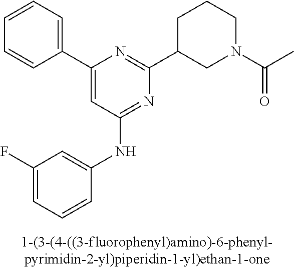 [SMILES: CC(=O)N1CCCC(c2nc(Nc3cccc(F)c3)cc(-c3ccccc3)n2)C1] | ¹H-NMR (400 MHz, DMSO-d6) mixture of rotamers δ 9.91 (d, J = 10.5 Hz, 1H), 8.11-8.01 (m, 2H), 7.97-7.84 (m, 1H), 7.61- 7.49 (m, 3H), 7.45-7.31 (m, 2H), 7.13 (d, J = 4.2 Hz, 1H), 6.83 (t, J = 8.5 Hz, 1H), 4.81- 4.71 (m, 0.5H), 4.28-4.11 (m, 1H), 3.92-3.80 (m, 0.5H), 3.55- 3.46 (m, 0.5H), 3.15-3.02 (m, 0.5H), 3.01-2.90 (m, 0.5H), 2.89-2.72 (m, 1.5H), 2.29-2.20 (m, 1H), 2.05 (s, 3H), 1.95- 1.73 (m, 2H), 1.68-1.40 (m, 1H) ); LCMS (Method D): t_{R} 3.63 min, 100%, MS (ESI) 391.1 (M + H)⁺ |
| 00145 | 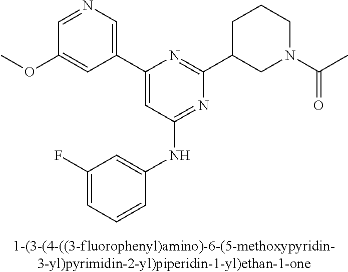 [SMILES: COc1cncc(-c2cc(Nc3cccc(F)c3)nc(C3CCCN(C(C)=O)C3)n2)c1] | ¹H-NMR (400 MHz, DMSO-d6) mixture of rotamers δ 10.02 (s, 1H), 8.81 (dd, J = 6.6, 1.7 Hz, 1H), 8.43 (t, J = 2.6 Hz, 1H), 7.97-7.82 (m, 2H), 7.48-7.31 (m, 2H), 7.20 (d, J = 6.0 Hz, 1H), 6.84 (t, J = 8.5 Hz, 1H), 4.74-4.67 (m, 0.5H), 4.25-4.09 (m, 1H), 3.93 (S, 3H), 3.84 (d, J = 13.5 Hz, 0.5H), 3.52 (dd, J = 13.5, 10.1 Hz, 0.5H), 3.16- 3.04 (m, 0.5H), 3.03-2.85 (m, 1H), 2.86-2.74 (m, 1H), 2.21 (s, 1H), 2.04 (s, 3H), 1.98-1.71 (m, 2H), 1.67-1.40 (m, 1H); LCMS (Method D): t_{R} 3.30 min, 100%, MS (ESI) 422.2 |
| | | (M + H)⁺ |
| 00146 | 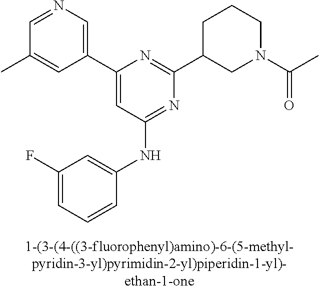 [SMILES: CC(=O)N1CCCC(c2nc(Nc3cccc(F)c3)cc(-c3cncc(C)c3)n2)C1] | ¹H-NMR (400 MHz, DMSO-d6) mixture of rotamers δ 9.96 (d, J = 10.1 Hz, 1H), 9.00 (dd, J = 6.6, 2.1 Hz, 1H), 8.55 (s, 1H), 8.20 (s, 1H), 7.95-7.81 (m, 1H), 7.48-7.31 (m, 2H), 7.17 (d, J = 4.1 Hz, 1H), 6.84 (t, J = 8.5 Hz, 1H), 4.74 (d, J = 12.4 Hz, 0.5H), 4.23 (d, J = 13.1 Hz, 0.5H), 4.14 (d, J = 13.9 Hz, 0.5H), 3.86 (d, J = 13.6 Hz, 0.5H), 3.51 (dd, J = 13.4, 10.2 Hz, 0.5H), 3.09 (t, J = 12.6 Hz, 0.5H), 3.01-2.92 (m, 0.5H), 2.86 (d, J = 12.1 Hz, 0.5H), 2.77 (t, J = 11.9 Hz, 1H), 2.41 (s, 3H), 2.23 (d, J = 12.4 Hz, 1H), 2.05 (s, 3H), |
| | | 1.95-1.72 (m, 2H), 1.64-1.40 |
| | | (m, 1H); LCMS (Method D): |
| | | t_{R} 3.31 min, 100%, MS (ESI) |
| | | 406.2 (M + H)⁺ |
| 00147 | 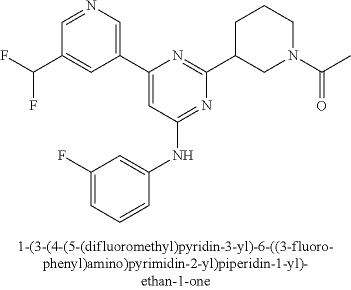 [SMILES: CC(=O)N1CCCC(c2nc(Nc3cccc(F)c3)cc(-c3cncc(C(F)F)c3)n2)C1] | ¹H-NMR (400 MHz, DMSO-d6) mixture of rotamers δ 10.03 (d, J = 9.9 Hz, 1H), 9.36 (d, J = 6.5 Hz, 1H), 8.93 (s, 1H), 8.57 (s, 1H), 7.89 (dd, J = 13.5, 11.1 Hz, 1H), 7.48-7.13 (m, 4H), 6.86 (t, J = 8.4 Hz, 1H), 4.74 (d, J = 12.1 Hz, 0.5H), 4.24 (d, J = 12.9 Hz, 0.5H), 4.15 (d, J = 13.4 Hz, 0.5H), 3.86 (d, J = 13.5 Hz, 0.5H), 3.51 (dd, J = 13.5, 10.3 Hz, 0.5H), 3.09 (t, J = 12.3 Hz, 0.5H), 3.04-2.93 (m, 0.5H), 2.92-2.70 (m, 1.5H), 2.24 (d, J = 12.6 Hz, 1H), 2.05 (S, 3H), 1.94-1.72 (m, 2H), 1.65-1.41 (m, 1H); LCMS (Method D): t_{R} 3.40 min, 100%, MS (ESI) |
| | | 442.1 (M + H)⁺ |

### Example 5: Synthesis of cyclopropyl(3-(4-((3-fluorophenyl)amino)-6-(pyridin-3-yl)pyrimidin-2-yl)piperidin-1-yl)methanone (00148)

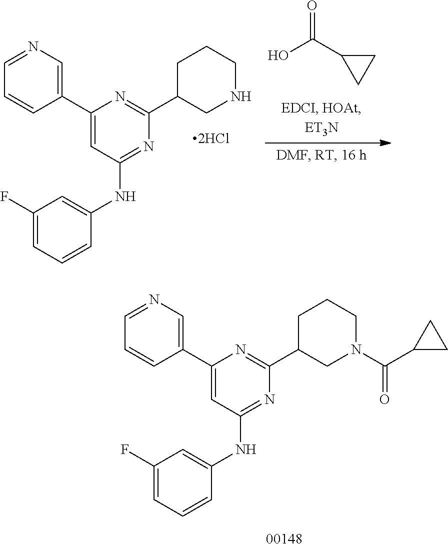 [SMILES: Fc1cccc(Nc2cc(-c3cccnc3)nc(C3CCCNC3)n2)c1 ; O=C(O)C1CC1 ; O=C(C1CC1)N1CCCC(c2nc(Nc3cccc(F)c3)cc(-c3cccnc3)n2)C1]

To a solution of N-(3-fluorophenyl)-2-(piperidin-3-yl)-6-(pyridin-3-yl)pyrimidin-4-amine dihydrochloride (15 mg, 0.04 mmol), cyclopropanecarboxylic acid (3.40 μL, 0.04 mmol) and triethylamine (0.02 mL, 0.14 mmol) in degassed N,N-dimethylformamide (0.5 mL), was added HOAt (0.97 mg, 7.10 μmol) and N-(3-dimethylaminopropyl)-N-ethylcarbodiimide hydrochloride (8.17 mg, 0.04 mmol). The mixture was stirred at room temperature for 16 hours and purified by reversed phase chromatography (method A) to afford cyclopropyl(3-(4-((3-fluorophenyl)amino)-6-(pyridin-3-yl)pyrimidin-2-yl)piperidin-1-yl)methanone (9 mg, 61%) as a white solid. ¹H-NMR (400 MHz, DMSO-d6) mixture of rotamers δ 10.05-9.89 (m, 1H), 9.21 (s, 1H), 8.76-8.65 (m, 1H), 8.45-8.33 (m, 1H), 7.97-7.79 (m, 1H), 7.62-7.53 (m, 1H), 7.47-7.32 (m, 2H), 7.18 (s, 1H), 6.90-6.77 (m, 1H), 4.80-4.66 (m, 0.5H), 4.57-4.52 (m, 0.5H), 4.33-4.10 (m, 1H), 3.72-3.60 (m, 0.5H), 3.23-3.12 (m, 0.5H), 3.06-2.73 (m, 2H), 2.29-2.17 (m, 1H), 2.10-1.70 (m, 3H), 1.67-1.39 (m, 1H), 0.80-0.47 (m, 4H); LCMS (Method D): t_{R} 3.48 min, 100%, MS (ESI) 418.1 (M+H)⁺.

The following compounds were prepared using procedures analogous to Example 5.

| Compound | | |
|---|---|---|
| # | Structure and compound name | Analytical data |
| 00149 | 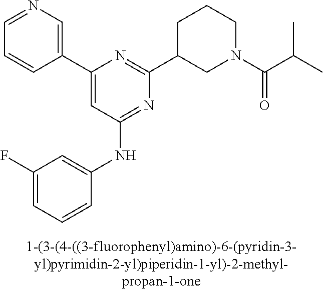 [SMILES: CC(C)C(=O)N1CCCC(c2nc(Nc3cccc(F)c3)cc(-c3cccnc3)n2)C1] | ¹H-NMR (400 MHz, DMSO-d6) mixture of rotamers δ 10.00 (s, 1H), 9.21 (s, 1H), 8.71 (d, J = 4.7 Hz, 1H), 8.43-8.34 (m, 1H), 7.99-7.82 (m, 1H), 7.63-7.54 (m, 1H), 7.48-7.32 (m, 2H), 7.22-7.13 (m, 1H), 6.92-6.78 (m, 1H), 4.82-4.72 (m, 0.5H), 4.34-4.21 (m, 1H), 4.03-3.94 (m, 0.5H), 3.52-3.41 (m, 0.5H), 3.15-3.05 (m, 0.5H), 3.03-2.85 (m, 2H), 2.84-2.70 (m, 1H), 2.30-2.17 (m, 1H), 2.02-1.72 (m, 2H), 1.65-1.37 (m, 1H), 1.11-0.88 (m, 6H); LCMS (Method D): t_{R} 3.55 min, 100%, MS (ESI) 420.2 (M + H)⁺. |
| 00150 | 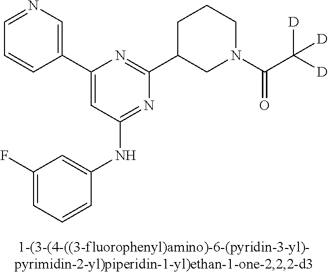 [SMILES: [2H]C([2H])([2H])C(=O)N1CCCC(c2nc(Nc3cccc(F)c3)cc(-c3cccnc3)n2)C1] | ¹H-NMR (400 MHz, DMSO-d6) mixture of rotamers δ 10.05- 9.92 (m, 1H), 9.26-9.15 (m, 1H), 8.75-8.66 (m, 1H), 8.44-8.33 (m, 1H), 7.94-7.83 (m, 1H), 7.62-7.52 (m, 1H), 7.47-7.31 (m, 2H), 7.18 (d, J = 4.5 Hz, 1H), 6.90-6.79 (m, 1H), 4.79- 4.71 (m, 0.5H), 4.29-4.09 (m, 1H), 3.90-3.80 (m, 0.5H), 3.55- 3.45 (m, 0.5H), 3.13-3.03 (m, 0.5H), 3.02-2.92 (m, 0.5H), 2.92-2.83 (m, 0.5H), 2.83-2.71 (m, 1H), 2.29-2.18 (m, 1H), 1.96-1.71 (m, 2H), 1.67-1.37 (m, 1H); LCMS (Method D): t_{R} 3.24 min, 100%, MS (ESI) |
| | | 395.2 (M + H)⁺. |
| 00151 | 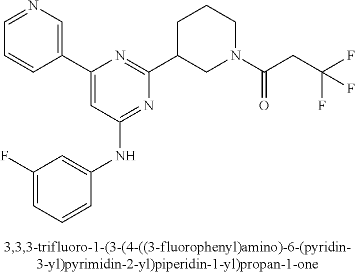 [SMILES: O=C(CC(F)(F)F)N1CCCC(c2nc(Nc3cccc(F)c3)cc(-c3cccnc3)n2)C1] | ¹H-NMR (400 MHz, DMSO-d6) mixture of rotamers δ 9.99 (d, J = 5.7 Hz, 1H), 9.22 (dd, J = 7.8, 2.3 Hz, 1H), 8.71 (dt, J = 4.7, 1.8 Hz, 1H), 8.39 (dt, J = 8.0, 2.0 Hz, 1H), 7.86 (dt, J = 12.1, 2.3 Hz, 1H), 7.58 (dd, J = 8.1, 4.9 Hz, 1H), 7.48-7.33 (m, 2H), 7.18 (s, 1H), 6.90-6.78 (m, 1H), 4.79-4.68 (m, 0.5H), 4.26-4.16 (m, 0.5H), 4.15-4.05 (m, 0.5H), 3.87 (d, J = 13.6 Hz, 0.5H), 3.81-3.50 (m, 2H), 3.15- 2.75 (m, 2H), 2.29-2.14 (m, 1H), 1.99-1.72 (m, 2H), 1.69- 1.42 (m, 1H); LCMS (Method D): t_{R} 3.56 min, 100%, MS |
| | | (ESI) 460.1 (M + H)⁺. |
| 00152 | 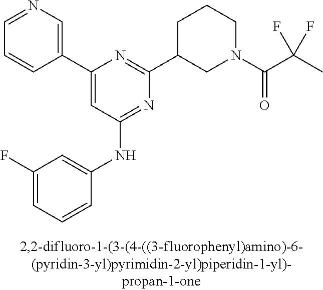 [SMILES: CC(F)(F)C(=O)N1CCCC(c2nc(Nc3cccc(F)c3)cc(-c3cccnc3)n2)C1] | ¹H-NMR (400 MHz, DMSO-d6) mixture of rotamers δ 10.01 (d, J = 6.3 Hz, 1H), 9.21 (d, J = 2.2 Hz, 1H), 8.79-8.60 (m, 1H), 8.46-8.33 (m, 1H), 7.93-7.79 (m, 1H), 7.66-7.52 (m, 1H), 7.48-7.42 (m, 1H), 7.42-7.32 (m, 1H), 7.22-7.16 (m, 1H), 6.89-6.79 (m, 1H), 4.73-4.58 (m, 1H), 4.40 (m, J = 12.8 Hz, 0.5H), 4.11 (m, J = 13.7 Hz, 0.5H), 3.41 (m, J = 12.1 Hz, 1H), 3.20 (m, J = 27.5, 15.8 Hz, 1H), 2.95 (m, J = 15.7, 10.7 Hz, 1H), 2.81 (m, J = 11.5 Hz, 0.5H), 2.40-2.21 (m, 1H), 1.99-1.71 (m, 5.5H), 1.61 (m, J = 16.7, 15.5 Hz, |
| | | 1H); LCMS (Method D): t_{R} |
| | | 3.74 min, 100%, MS (ESI) |
| | | 442.2 (M + H)⁺. |
| 00153 | 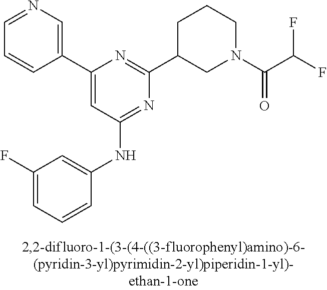 [SMILES: O=C(C(F)F)N1CCCC(c2nc(Nc3cccc(F)c3)cc(-c3cccnc3)n2)C1] | ¹H-NMR (400 MHz, DMSO-d6) mixture of rotamers δ 10.00 (d, J = 5.5 Hz, 1H), 9.26-9.18 (m, 1H), 8.71 (d, J = 4.7 Hz, 1H), 8.39 (dd, J = 8.1, 2.2 Hz, 1H), 7.91-7.82 (m, 1H), 7.58 (dd, J = 7.9, 4.7 Hz, 1H), 7.46-7.33 (m, 2H), 7.19 (s, 1H), 6.99-6.63 (m, 2H), 4.71-4.63 (m, 0.5H), 4.34- 4.20 (m, 1H), 3.96-3.87 (m, 0.5H), 3.55 (dd, J = 13.7, 10.6 Hz, 0.5H), 3.26-3.11 (m, 1H), 3.05-2.96 (m, 0.5H), 2.96-2.83 (m, 1H), 2.34-2.22 (m, 1H), 1.97-1.79 (m, 2H), 1.71-1.48 (m, 1H); LCMS (Method D): t_{R} 3.44 min, 100%, MS (ESI) 428.1 (M + H)⁺. |
| 00154 | 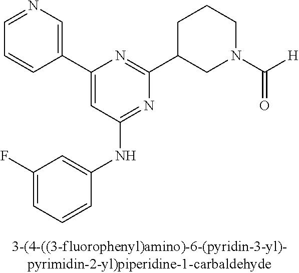 [SMILES: O=CN1CCCC(c2nc(Nc3cccc(F)c3)cc(-c3cccnc3)n2)C1] | ¹H-NMR (400 MHz, DMSO-d6) mixture of rotamers δ 10.00 (2s, 1H), 9.22 (t, J = 3.1 Hz, 1H), 8.71 (d, J = 4.8 Hz, 1H), 8.45- 8.37 (m, 1H), 8.08 (d, J = 4.6 Hz, 1H), 7.91-7.80 (m, 1H), 7.58 (dd, J = 8.0, 4.8 Hz, 1H), 7.48-7.34 (m, 2H), 7.18 (s, 1H), 6.91-6.81 (m, 1H), 4.57-4.45 (m, 0.5H), 4.05-3.94 (m, 1H), 3.75-3.66 (m, 0.5H), 3.57 (dd, J = 13.1, 9.8 Hz, 0.5H), 3.17- 3.01 (m, 1H), 2.98-2.75 (m, 1.5H), 2.32-2.21 (m, 1H), 2.03- 1.79 (m, 1.5H), 1.80-1.69 (m, 0.5H), 1.64-1.39 (m, 1H); LCMS (Method D): t_{R} 3.16 |
| | | min, 100%, MS (ESI) 378.2 |
| | | (M + H)⁺. |
| 00155 | 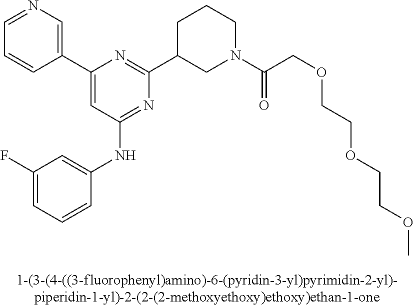 [SMILES: COCCOCCOCC(=O)N1CCCC(c2nc(Nc3cccc(F)c3)cc(-c3cccnc3)n2)C1] | ¹H-NMR (400 MHz, DMSO-d6) mixture of rotamers δ 10.02 (d, J = 7.0 Hz, 1H), 9.22 (s, 1H), 8.71 (d, J = 4.8 Hz, 1H), 8.39 (dt, J = 8.1, 2.1 Hz, 1H), 7.87 (dt, J = 12.2, 2.3 Hz, 1H), 7.58 (dd, J = 8.0, 4.7 Hz, 1H), 7.51- 7.32 (m, 2H), 7.19 (d, J = 2.7 Hz, 1H), 6.85 (t, J = 8.4 Hz, 1H), 4.67 (d, J = 12.7 Hz, 0.5H), 4.31-4.07 (m, 3H), 3.83 (d, J = 13.4 Hz, 0.5H), 3.62- 3.46 (m, 6H), 3.47-3.40 (m, 3H), 3.24 (s, 2H), 3.15 (s, 1.5H), 3.09-2.93 (m, 1.5H), 2.88-2.70 (m, 1H), 2.23 (d, J = 12.8 Hz, 1H), 1.95-1.75 (m, 2H), 1.70-1.45 (m, 1H); LCMS (Method D): t_{R} 3.18 min, 100%, |
| | | MS (ESI) 510.2 (M + H)⁺ |

### Example 6: Synthesis of 1-(3-(4-(cyclopropylamino)-6-((3-fluorophenyl)amino)pyrimidin-2-yl)piperidin-1-yl)propan-1-one (00156)

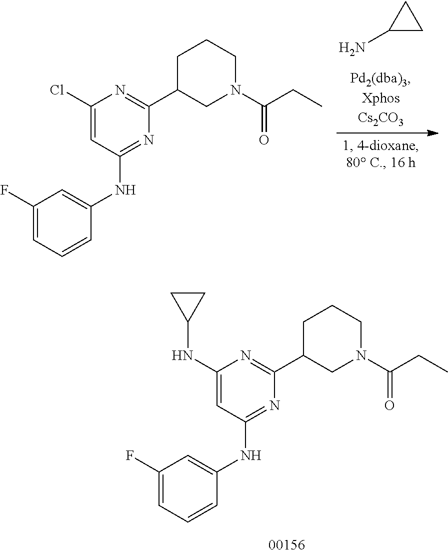

Under argon atmosphere, a solution of 1-(3-(4-chloro-6-((3-fluorophenyl)amino)pyrimidin-2-yl)piperidin-1-yl)propan-1-one (20 mg, 0.06 mmol), cyclopropylamine (4.58 μL, 0.07 mmol), cesium carbonate (26.9 mg, 0.08 mmol), XPhos (1.31 mg, 2.76 μmol; CAS number 564483-18-7) and Pd₂(dba)₃ (1.26 mg, 1.38 μmol; CAS number 51364-51-3) in 1,4-dioxane (2 mL) was heated to 80° C. for 16 hours. The mixture was partitioned between water and dichloromethane and the layers were separated. The organic layer was concentrated and purified by reversed phase chromatography (method A) to afford 1-(3-(4-(cyclopropylamino)-6-((3-fluorophenyl)amino)pyrimidin-2-yl)piperidin-1-yl)propan-1-one (2.5 mg, 11%) as a white solid. ¹H-NMR (400 MHz, DMSO-d6) mixture of rotamers δ 9.31 (d, J=7.7 Hz, 1H), 7.81 (t, J=14.9 Hz, 1H), 7.40-7.22 (m, 2H), 7.14 (d, J=2.3 Hz, 1H), 6.70 (d, J=7.3 Hz, 1H), 5.86 (s, 1H), 4.70 (d, J=12.8 Hz, 0.5H), 4.29 (d, J=13.0 Hz, 0.5H), 4.06 (d, J=14.3 Hz, 0.5H), 3.88 (d, J=13.9 Hz, 0.5H), 3.00-2.87 (m, 0.5H), 2.73-2.56 (m, 2H), 2.46-2.30 (m, 5H), 2.09 (d, J=13.0 Hz, 1H), 1.83-1.63 (m, 2H), 1.56-1.31 (m, 1H), 1.05-0.93 (m, 4H), 0.74-0.65 (m, 2H), 0.53-0.43 (m, 2H); LCMS (Method D): t_{R} 3.42 min, 100%, MS (ESI) 384.2 (M+H)⁺.

The following compounds were prepared using procedures analogous to Example 6:

| Compound | | |
|---|---|---|
| # | Structure and compound name | Analytical data |
| 00157 | 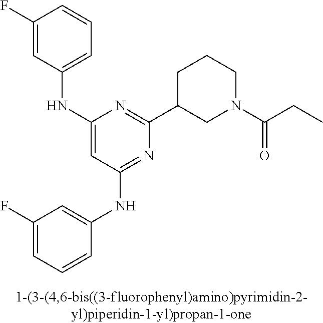 [SMILES: CCC(=O)N1CCCC(c2nc(Nc3cccc(F)c3)cc(Nc3cccc(F)c3)n2)C1] | ¹H-NMR (400 MHz, DMSO-d6) δ 9.45 (d, J = 11.4 Hz, 2H), 7.74 (dd, J = 29.2, 12.5 Hz, 2H), 7.37-7.23 (m, 4H), 6.81-6.68 (m, 2H), 6.10 (d, J = 3.9 Hz, 1H), 4.55 (dd, J = 183.0, 12.6 Hz, 1H), 4.06 (dd, J = 125.4, 13.5 Hz, 1H), 3.29-3.20 (m, 1H), 2.83-2.56 (m, 2H), 2.42- 2.31 (m, 2H), 2.20 (t, J = 14.5 Hz, 1H), 1.89-1.67 (m, 2H), 1.62-1.37 (m, 1H), 1.01 (t, J = 7.3 Hz, 3H); LCMS (Method B): t_{R} 3.50 min, 100%, MS (ESI) 438.2 (M + H)⁺. |
| 00158 | 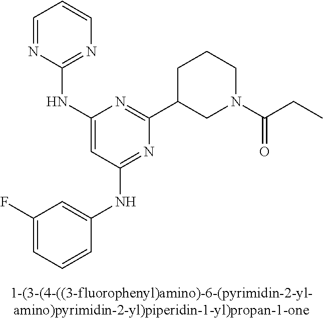 [SMILES: CCC(=O)N1CCCC(c2nc(Nc3cccc(F)c3)cc(Nc3ncccn3)n2)C1] | ¹H-NMR (400 MHz, DMSO-d6) mixture of rotamers δ 9.85 (s, 1H), 9.74 (d, J = 8.2 Hz, 1H), 8.65-8.58 (m, 2H), 7.95-7.81 (m, 1H), 7.66 (s, 1H), 7.41 (t, J = 8.3 Hz, 1H), 7.37-7.26 (m, 1H), 7.05 (t, J = 5.0 Hz, 1H), 6.76 (t, J = 8.6 Hz, 1H), 4.75 (d, J = 12.5 Hz, 0.5H), 4.30 (d, J = 13.0 Hz, 0.5H), 4.15 (d, J = 13.0 Hz, 0.5H), 3.91 (d, J = 14.2 Hz, 0.5H), 3.40-3.34 (m, 0.5H), 3.08-2.91 (m, 0.5H), 2.82-2.59 (m, 2H), 2.41-2.29 (m, 4H), 2.22-2.11 (m, 1H), 1.87-1.70 (m, 2H), 1.59-1.35 (m, 1H), 1.05-0.96 (m, 3H) ); LCMS (Method D): t_{R} 3.48 min, 100%, MS (ESI) 422.2 (M + H)⁺ |
| 00159 | 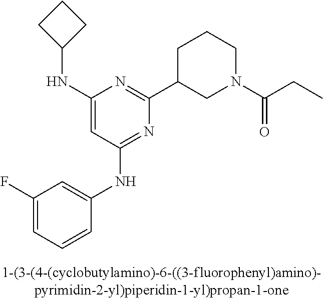 [SMILES: CCC(=O)N1CCCC(c2nc(Nc3cccc(F)c3)cc(NC3CCC3)n2)C1] | ¹H-NMR (400 MHz, DMSO-d6) mixture of rotamers δ 9.16 (s, 1H), 8.31 (s, 0H), 7.83-7.63 (m, 1H), 7.52-7.13 (m, 3H), 6.72- 6.63 (m, 1H), 5.56 (s, 1H), 4.69 (d, J = 12.7 Hz, 0.5H), 4.25 (s, 0.5H), 4.06 (d, J = 12.1 Hz, 0.5H), 3.88 (d, J = 13.8 Hz, 0.5H), 3.03-2.89 (m, 0.5H), 2.73-2.55 (m, 1H), 2.46-2.39 (m, 1H), 2.39-2.30 (m, 3H), 2.30-2.20 (m, 2H), 2.15-2.05 (m, 1H), 1.98-1.86 (m, 2H), 1.84-1.61 (m, 4H), 1.56-1.32 (m, 1H), 1.05-0.95 (m, 3H); LCMS (Method D): t_{R} 3.60 min, 100%, MS (ESI) 398.2 (M + H)⁺. |
| 00160 | 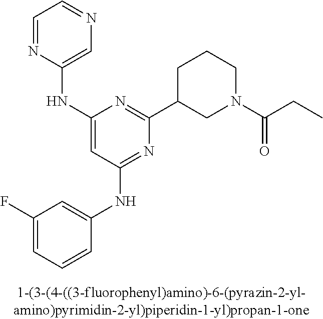 [SMILES: CCC(=O)N1CCCC(c2nc(Nc3cccc(F)c3)cc(Nc3cnccn3)n2)C1] | ¹H-NMR (400 MHz, DMSO-d6) mixture of rotamers δ 10.20 (d, J = 2.9 Hz, 1H), 9.71 (d, J = 8.3 Hz, 1H), 8.93 (d, J = 11.5 Hz, 1H), 8.28 (s, 1H), 8.18-8.12 (m, 1H), 7.89-7.77 (m, 1H), 7.34 (dq, J = 23.0, 8.1 Hz, 2H), 7.17 (d, J = 8.7 Hz, 1H), 6.76 (t, J = 8.4 Hz, 1H), 4.79 (d, J = 12.2 Hz, 0.5H), 4.35 (d, J = 12.8 Hz, 0.5H), 4.19 (d, J = 13.3 Hz, 0.5H), 3.91 (d, J = 13.8 Hz, 0.5H), 3.00 (t, J = 12.5 Hz, 0.5H), 2.87-2.58 (m, 2H), 2.41- 2.29 (m, 4H), 2.18 (s, 1H), 1.80 (t, J = 16.6 Hz, 2H), 1.50 (d, J = 41.3 Hz, 1H), 1.05-0.97 (m, 3H); LCMS (Method D): t_{R} 3.34 min, 100%, MS (ESI) 422.2 (M + H)⁺ |
| 00161 | 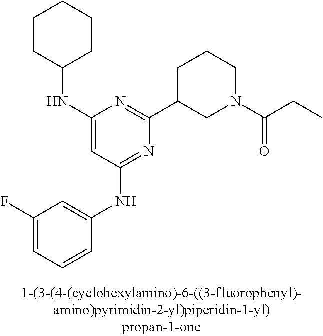 [SMILES: CCC(=O)N1CCCC(c2nc(Nc3cccc(F)c3)cc(NC3CCCCC3)n2)C1] | ¹H-NMR (400 MHz, DMSO-d6) mixture of rotamers δ 9.11 (s, 1H), 7.86-7.63 (m, 1H), 7.31- 7.18 (m, 2H), 6.85-6.72 (m, 1H), 6.72-6.63 (m, 1H), 5.64 (s, 1H), 4.68 (d, J = 12.7 Hz, 0.5H), 4.25 (d, J = 12.7 Hz, 0.5H), 4.07 (dd, J = 13.4, 3.6 Hz, 0.5H), 3.87 (d, J = 13.6 Hz, 0.5H), 3.31-3.22 (m, 0.5H), 2.95 (t, J = 12.7 Hz, 0.5H), 2.73-2.57 (m, 1.5H), 2.47-2.40 (m, 0.5H), 2.38-2.30 (m, 2H), 2.09 (d, J = 12.5 Hz, 1H), 1.89 (d, J = 12.1 Hz, 2H), 1.81-1.08 (m, 13H), 1.03-0.95 (m, 3H); LCMS (Method D): t_{R} 3.87 min, 100%, MS (ESI) 426.2 (M + H)⁺ |
| 00162 | 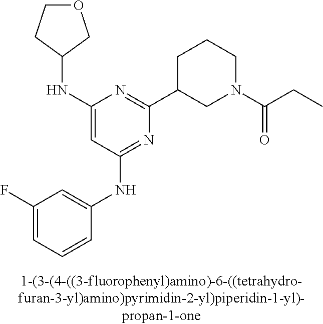 [SMILES: CCC(=O)N1CCCC(c2nc(Nc3cccc(F)c3)cc(NC3CCOC3)n2)C1] | ¹H-NMR (400 MHz, DMSO-d6) mixture of rotamers δ 9.17 (d, J = 9.7 Hz, 1H), 7.72 (dd, J = 22.6, 12.5 Hz, 1H), 7.34-7.20 (m, 2H), 7.14 (s, 1H), 6.77-6.62 (m, 1H), 5.69 (s, 1H), 4.69 (d, J = 11.5 Hz, 0.5H), 4.41-4.20 (m, 1H), 4.08 (d, J = 12.9 Hz, 0.5H), 3.93-3.67 (m, 4H), 3.59- 3.49 (m, 1H), 2.96 (t, J = 12.2 Hz, 0.5H), 2.74-2.57 (m, 2H), 2.39-2.30 (m, 3H), 2.23-2.05 (m, 3H), 1.88-1.64 (m, 4H), 1.59-1.33 (m, 1H), 1.03-0.94 (m, 4H); LCMS (Method D): t_{R} 3.23 min, 100%, MS (ESI) 414.2 (M + H)⁺ |

The following further compounds were prepared using procedures analogous to Example 6.

| Compound | | |
|---|---|---|
| # | Structure and compound name | Analytical data |
| 00163 | 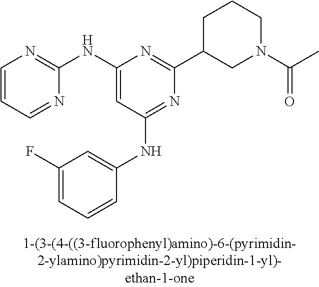 [SMILES: CC(=O)N1CCCC(c2nc(Nc3cccc(F)c3)cc(Nc3ncccn3)n2)C1] | 1H-NMR (400 MHz, DMSO-d6) mixture of rotamers δ 9.84 (d, J = 3.0 Hz, 1H), 9.74 (d, J = 8.6 Hz, 1H), 8.62 (dd, J = 4.8, 2.0 Hz, 2H), 7.99-7.81 (m, 1H), 7.66 (s, 1H), 7.41 (d, J = 8.4 Hz, 1H), 7.39-7.28 (m, 1H), 7.16- 6.99 (m, 1H), 6.76 (t, J = 8.5 Hz, 1H), 4.73 (d, J = 12.7 Hz, 0.5H), 4.26 (d, J = 12.8 Hz, 0.5H), 4.10 (d, J = 13.5 Hz, 0.5H), 3.86 (d, J = 13.7 Hz, 0.5H), 3.44 (m, 0.5H), 3.13-2.95 (m, 0.51 H), 2.84-2.65 (m, 2H), 2.24-2.12 (m, 1H), 2.04 (d, J = 1.9 Hz, 3H), 1.92-1.70 (m, 2H), 1.62-1.36 (m, 1H); LCMS (Method D): t_{R} 3.60 min, 98%, MS (ESI) 408.2 (M + H)+ |
| 00164 | 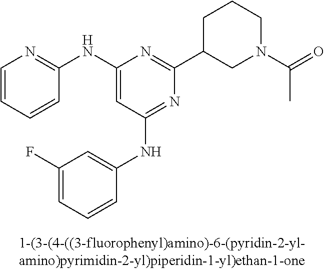 [SMILES: CC(=O)N1CCCC(c2nc(Nc3cccc(F)c3)cc(Nc3ccccn3)n2)C1] | 1H-NMR (400 MHz, DMSO-d6) mixture of rotamers δ 9.91-9.78 (m, 1H), 9.71-9.54 (m, 1H), 8.36-8.20 (m, 1H), 7.93-7.76 (m, 1H), 7.77-7.63 (m, 1H), 7.64- 7.45 (m, 1H), 7.45-7.21 (m, 3H), 7.03-6.86 (m, 1H), 6.79-6.62 (m, 1H), 4.84-4.70 (m, 0.5H), 4.39- 4.23 (m, 0.5H), 4.21-4.06 (m, 0.5H), 4.00-3.79 (m, 0.5H), 3.42- 3.36 (m, 0.5H), 3.10-2.96 (m, 0.5H), 2.88-2.55 (m, 2H), 2.26- 2.12 (m, 1H), 2.04 (s, 3H), 1.95- 1.67 (m, 2H), 1.64-1.34 (m, 1H); LCMS (Method D): t_{R} 3.75 min, 100%, MS (ESI) 407.1 (M + H)+ |
| 00165 | 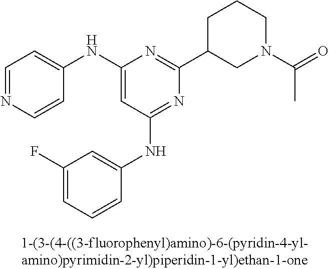 [SMILES: CC(=O)N1CCCC(c2nc(Nc3ccncc3)cc(Nc3cccc(F)c3)n2)C1] | ¹H-NMR (400 MHz, DMSO-d6) mixture of rotamers δ 9.82-9.74 (m, 1H), 9.62-9.54 (m, 1H), 8.40-8.31 (m, 2H), 7.80-7.64 (m, 3H), 7.38-7.27 (m, 2H), 6.82-6.74 (m, 1H), 6.19 (s, 1H), 4.80-4.71 (m, 0.5H), 4.34-4.25 (m, 0.5H), 4.20-4.13 (m, 0.5H), 3.92-3.81 (m, 0.5H), 3.41-3.35 (m, 0.5H), 3.19-3.14 (m, 0.5H), 3.11-3.00 (m, 0.5H), 2.89-2.60 (m, 2H), 2.27-2.14 (m, 1H), 2.08-1.99 (m, 3H), 1.87-1.70 (m, 2H), 1.66-1.41 (m, 1H), 1.27-1.18 (m, 1H); LCMS (Method D): t_{R} 3.45 min, 100%, MS (ESI) 407.2 (M + H)+ |
| 00166 | 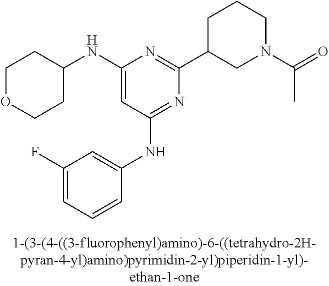 [SMILES: CC(=O)N1CCCC(c2nc(Nc3cccc(F)c3)cc(NC3CCOCC3)n2)C1] | ¹H-NMR (400 MHz, DMSO-d6) mixture of rotamers δ 9.25-9.06 (m, 1H), 7.84-7.62 (m, 1H), 7.33-7.19 (m, 2H), 6.97-6.85 (m, 1H), 6.73-6.62 (m, 1H), 5.67 (s, 1H), 4.69-4.59 (m, 0.5H), 4.30-4.17 (m, 0.5H), 4.12-3.97 (m, 0.5H), 3.97-3.70 (m, 3H), 3.45-3.34 (m, 4H), 3.07-2.92 (m, 0.5H), 2.72-2.57 (m, 1.5H), 2.49-2.40 (m, 0.5H), 2.16-2.05 (m, 1H), 1.93-1.81 (m, 2H), 1.81-1.60 (m, 2H), 1.59-1.31 (m, 3.5H), 1.23 (s, 1.5H); LCMS (Method D): t_{R} 3.45 min, 99%, MS (ESI) 414.2 (M + H)⁺ |
| 00167 | 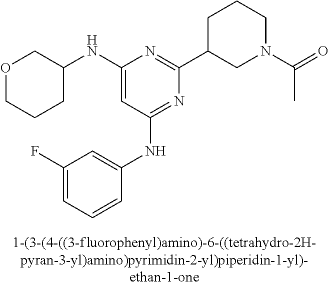 [SMILES: CC(=O)N1CCCC(c2nc(Nc3cccc(F)c3)cc(NC3CCCOC3)n2)C1] | ¹H-NMR (400 MHz, DMSO-d6) mixture of rotamers δ 9.15 (d, J = 9.1 Hz, 1H), 7.70 (t, J = 13.7 Hz, 1H), 7.34-7.17 (m, 2H), 6.85 (d, J = 8.2 Hz, 1H), 6.75-6.63 (m, 1H), 5.72 (s, 1H), 4.74-4.58 (m, 0.5H), 4.24 (d, J = 12.9 Hz, 0.5H), 4.07-3.97 (m, 0.5H), 3.95-3.78 (m, 0.5H), 3.72 (dd, J = 9.6, 5.7 Hz, 1H), 3.29 (dd, J = 6.7, 4.0 Hz, 0.5H), 3.12-2.94 (m, 1.5H), 2.73-2.58 (m, 1.5H), 2.49-2.41 (m, 0.5H), 2.15-2.05 (m, 1H), 2.01 (d, J = 2.3 Hz, 3H), 1.98-1.88 (m, 1H), 1.84- 1.32 (m, 6H), 1.23 (s, 1H); LCMS (Method D): t_{R} 3.55 min, 100%, MS (ESI) 414.2 |
| | | (M + H)⁺ |
| 00168 | 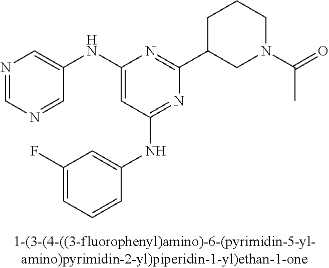 [SMILES: CC(=O)N1CCCC(c2nc(Nc3cncnc3)cc(Nc3cccc(F)c3)n2)C1] | ¹H-NMR (400 MHz, DMSO-d6) mixture of rotamers δ 9.64 (d, J = 8.0 Hz, 1H), 9.54 (d, J = 10.4 Hz, 1H), 9.11 (d, J = 3.7 Hz, 2H), 8.77 (d, J = 2.3 Hz, 1H), 7.87-7.63 (m, 1H), 7.42- 7.21 (m, 2H), 6.87-6.68 (m, 1H), 6.12 (s, 1H), 4.79-4.70 (m, 0.5H), 4.30 (d, J = 12.9 Hz, 0.5H), 4.22-4.08 (m, 0.5H), 3.85 (d, J = 13.5 Hz, 0.5H), 3.33-3.29 (m, 0.5H), 3.04 (m, 0.5H), 2.88-2.77 (m, 0.5H), 2.77-2.68 (m, 0.5H), 2.68-2.58 (m, 1H), 2.26-2.14 (m, 1H), 2.03 (s, 3H), 1.87-1.67 (m, 2H), 1.65-1.36 (m, 1H); LCMS |
| | | (Method D): t_{R} 3.32 min, 100%, |
| | | MS (ESI) 408.2 (M + H)+ |
| 00169 | 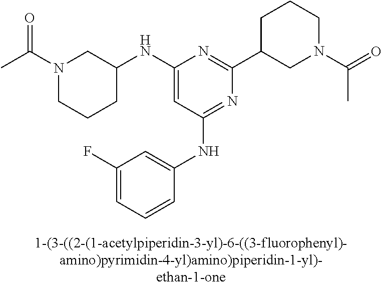 [SMILES: CC(=O)N1CCCC(Nc2cc(Nc3cccc(F)c3)nc(C3CCCN(C(C)=O)C3)n2)C1] | ¹H-NMR (400 MHz, DMSO-d6) mixture of rotamers δ δ 9.16 (d, J = 8.3 Hz, 1H), 7.84-7.58 (m, 1H), 7.38-7.15 (m, 2H), 6.93 (dt, J = 50.8, 7.4 Hz, 1H), 6.79-6.63 (m, 1H), 5.85-5.62 (m, 1H), 4.68 (d, J = 12.5 Hz, 0.5H), 4.53-4.19 (m, 1H), 4.14-3.99 (m, 0.5H), 3.94-3.61 (m, 2.5H), 3.39-3.25 (m, 0.5H), 3.15-2.83 (m, 2H), 2.76-2.42 (m, 2H), 2.19-1.86 (m, 8H), 1.83-1.64 (m, 3H), 1.64-1.30 (m, 3H); LCMS (Method D): t_{R} 3.35 min, 100%, MS (ESI) 455.2 (M + H)⁺ |
| 00170 | 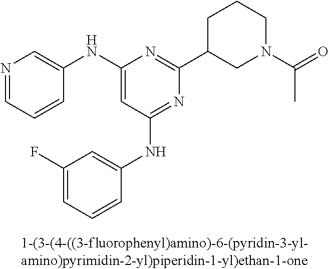 [SMILES: CC(=O)N1CCCC(c2nc(Nc3cccnc3)cc(Nc3cccc(F)c3)n2)C1] | ¹H-NMR (400 MHz, DMSO-d6) mixture of rotamers δ 9.50-9.38 (m, 2H), 8.82-8.75 (m, 1H), 8.20-8.07 (m, 2H), 7.81-7.67 (m, 1H), 7.37-7.25 (m, 3H), 6.80-6.71 (m, 1H), 6.09 (s, 1H), 4.78-4.69 (m, 0.5H), 4.34-4.24 (m, 0.5H), 4.18-4.08 (m, 0.5H), 3.91-3.76 (m, 0.5H), 3.39-3.28 (m, 1.5H), 3.11-2.97 (m, 0.5H), 2.84-2.55 (m, 2H), 2.24-2.12 (m, 1H), 2.03 (s, 3H), 1.85-1.67 (m, 2H), 1.64-1.39 (m, 1H); LCMS (Method D): t_{R} 3.47 min, 100%, MS (ESI) 407.2 (M + H)+ |
| 00171 | 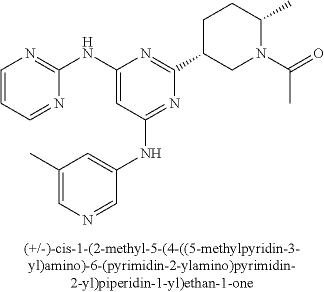 | ¹H-NMR (400 MHz, DMSO-d6) mixture of rotamers δ 9.96-9.88 (m, 1H), 9.75-9.62 (m, 1H), 8.70-8.56 (m, 3H), 8.22-8.12 (m, 1H), 8.06-7.98 (m, 1H), 7.72-7.63 (m, 1H), 7.08-7.00 (m, 1H), 4.85-4.76 (m, 0.5H), 4.70-4.60 (m, 0.5H), 4.26-4.13 (m, 0.5H), 3.99-3.88 (m, 0.5H), 3.46-3.37 (m, 0.5H), 2.90-2.79 (m, 0.5H), 2.77-2.65 (m, 0.5H), 2.62-2.51 (m, 0.5H), 2.30 (s, 3H), 2.10-1.58 (m, 7H), 1.26 (d, J = 6.8 Hz, 1.5H), 1.14 (d, J = 6.9 Hz, 1.5H); LCMS (Method B): t_{R} 2.31 min, 98%, MS (ESI) 407.2 (M + H)+ |

### Example 7: Synthesis of 1-(3-(4-(benzylamino)-6-((3-fluorophenyl)amino)pyrimidin-2-yl)piperidin-1-yl)propan-1-one (00172)

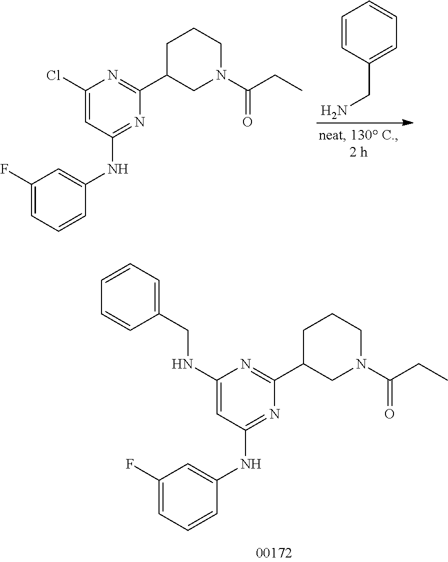

A microwave vial was charged with 1-(3-(4-chloro-6-((3-fluorophenyl)amino)pyrimidin-2-yl)piperidin-1-yl)propan-1-one (20 mg, 0.06 mmol) in benzylamine (1 mL, 9.15 mmol) and heated to 130° C. for 2 hours. The mixture was purified by reversed phase chromatography (method A) to afford 1-(3-(4-(benzylamino)-6-((3-fluorophenyl)amino)pyrimidin-2-yl)piperidin-1-yl)propan-1-one (8 mg, 32%) as a white solid. ¹H-NMR (400 MHz, DMSO-d6) mixture of rotamers δ 9.15 (d, J=9.0 Hz, 1H), 7.82-7.62 (m, 1H), 7.59-7.43 (m, 1H), 7.33 (d, J=4.4 Hz, 4H), 7.28-7.17 (m, 3H), 6.68 (t, J=8.5 Hz, 1H), 5.65 (s, 1H), 4.69 (d, J=12.7 Hz, 0.5H), 4.44 (s, 0.52H), 4.25 (d, J=13.0 Hz, 0.5H), 4.04 (d, J=13.0 Hz, 0.5H), 3.87 (d, J=13.5 Hz, 0.5H), 3.26 (m, 0.5H), 2.95 (t, J=12.8 Hz, 0.5H), 2.72-2.57 (m, 1.5H), 2.40-2.27 (m, 2H), 2.16-2.03 (m, 1H), 1.84-1.60 (m, 2H), 1.54-1.30 (m, 1H), 1.06-0.92 (m, 3H); LCMS (Method D): t_{R} 3.64 min, 100%, MS (ESI) 343.2 (M+H)⁺.

The following compounds was prepared using procedures analogous to Example 7:

| Compound | | |
|---|---|---|
| # | Structure and compound name | Analytical data |
| 00173 | 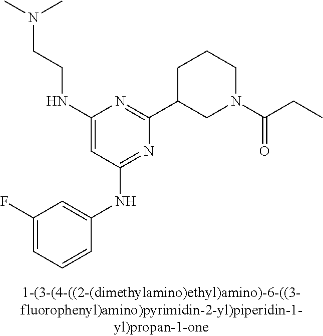 [SMILES: CCC(=O)N1CCCC(c2nc(NCCN(C)C)cc(Nc3cccc(F)c3)n2)C1] | ¹H-NMR (400 MHz, DMSO-d6) mixture of rotamers δ 9.23-9.06 (m, 1H), 7.73 (dd, J = 22.5, 12.6 Hz, 1H), 7.32-7.18 (m, 2H), 6.74 (s, 1H), 6.71-6.61 (m, 1H), 5.67 (s, 1H), 4.78-4.61 (m, 0.5H), 4.26 (d, J = 12.9 Hz, 0.5H), 4.07 (dd, J = 14.2, 3.3 Hz, 0.5H), 3.87 (d, J = 13.6 Hz, 0.5H), 3.26-3.18 (m, 0.5H), 2.95 (t, J = 12.1 Hz, 0.5H), 2.74-2.56 (m, 1.5H), 2.45-2.28 (m, 4H), 2.20 (s, 6H), 2.11 (dd, J = 16.6, 7.3 Hz, 1H), 1.86-1.63 (m, 2H), 1.56-1.33 (m, 1H), 1.06-0.94 (m, 3H); LCMS (Method D): t_{R} 3.39 min, 100%, MS (ESI) 415.3 (M + H)⁺. |

### Example 8: Synthesis of N-(3-fluorophenyl)-2-(1-isobutylpiperidin-3-yl)-6-(pyridin-3-yl)pyrimidin-4-amine (00174)

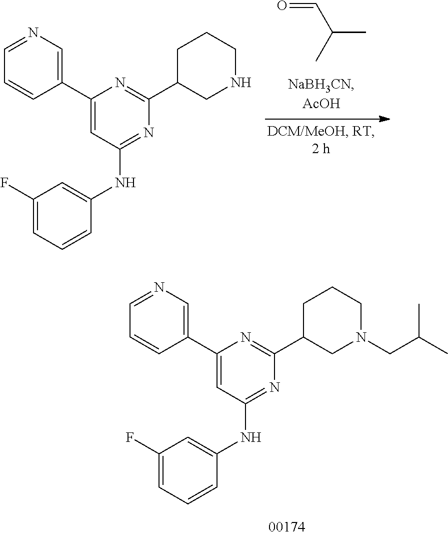

To a solution of N-(3-fluorophenyl)-2-(piperidin-3-yl)-6-(pyridin-3-yl)pyrimidin-4-amine (13 mg, 0.04 mmol) in dichloromethane (0.83 mL) and methanol (0.17 mL) was added acetic acid (2.13 μL, 0.04 mmol) and isobutyraldehyde (5.09 μl, 0.06 mmol) followed by sodium cyanoborohydride (3.51 mg, 0.06 mmol). The mixture was stirred at room temperature for 2 hours. Dichloromethane and saturated sodium carbonate solution were added and the layers were separated. The organic layer was concentrated, purified by reversed phase chromatography (method B) and lyophilized to afford N-(3-fluorophenyl)-2-(1-isobutylpiperidin-3-yl)-6-(pyridin-3-yl)pyrimidin-4-amine (9 mg, 60%) as a white solid. ¹H-NMR (400 MHz, DMSO-d6) δ 9.93 (s, 1H), 9.19 (d, J=2.3 Hz, 1H), 8.70 (d, J=4.7 Hz, 1H), 8.43-8.33 (m, 1H), 8.03-7.92 (m, 1H), 7.57 (dd, J=8.0, 4.7 Hz, 1H), 7.42-7.32 (m, 2H), 7.14 (s, 1H), 6.89-6.78 (m, 1H), 3.22-3.12 (m, 1H), 3.03-2.91 (m, 1H), 2.86-2.77 (m, 1H), 2.30-2.19 (m, 1H), 2.16-2.02 (m, 3H), 2.00-1.89 (m, 1H), 1.88-1.71 (m, 2H), 1.72-1.56 (m, 2H), 0.86 (t, J=7.1 Hz, 6H); LCMS (Method D): t_{R} 3.39 min, 100%, MS (ESI) 406.2 (M+H)⁺.

The following compounds were prepared using procedures analogous to Example 8:

| Compound | | |
|---|---|---|
| # | Structure and compound name | Analytical data |
| 00175 | 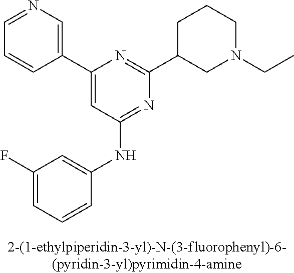 [SMILES: CCN1CCCC(c2nc(Nc3cccc(F)c3)cc(-c3cccnc3)n2)C1] | ¹H-NMR (400 MHz, DMSO-d6) δ 9.94 (s, 1H), 9.19 (d, J = 2.2 Hz, 1H), 8.70 (dd, J = 4.8, 1.6 Hz, 1H), 8.38 (dt, J = 7.9, 2.0 Hz, 1H), 8.02-7.91 (m, 1H), 7.57 (dd, J = 8.0, 4.8 Hz, 1H), 7.44-7.30 (m, 2H), 7.15 (s, 1H), 6.90-6.78 (m, 1H), 3.27-3.21 (m, 1H), 3.01-2.84 (m, 2H), 2.41 (q, J = 7.1 Hz, 2H), 2.29- 2.19 (m, 1H), 2.16-2.07 (m, 1H), 2.01-1.89 (m, 1H), 1.83- 1.72 (m, 1H), 1.71-1.55 (m, 2H), 1.04 (t, J = 7.2 Hz, 3H); LCMS (Method D): t_{R} 3.36 min, 100%, MS (ESI) 378.1 (M + H)⁺. |
| 00176 | 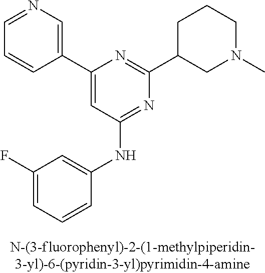 [SMILES: CN1CCCC(c2nc(Nc3cccc(F)c3)cc(-c3cccnc3)n2)C1] | ¹H-NMR (400 MHz, DMSO-d6) δ 9.95 (s, 1H), 9.19 (d, J = 2.2 Hz, 1H), 8.70 (dd, J = 4.8, 1.6 Hz, 1H), 8.38 (dt, J = 8.1, 2.0 Hz, 1H), 8.01-7.89 (m, 1H), 7.57 (dd, J = 8.0, 4.8 Hz, 1H), 7.45-7.31 (m, 2H), 7.15 (s, 1H), 6.90-6.78 (m, 1H), 3.18- 3.05 (m, 1H), 3.04-2.90 (m, 1H), 2.83-2.72 (m, 1H), 2.28- 2.15 (m, 4H), 2.13-2.03 (m, 1H), 1.96-1.85 (m, 1H), 1.81- 1.51 (m, 3H); LCMS (Method D): t_{R} 3.47 min, 100%, MS (ESI) 364.2 (M + H)⁺. |
| 00177 | 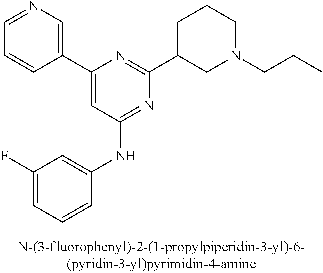 [SMILES: CCCN1CCCC(c2nc(Nc3cccc(F)c3)cc(-c3cccnc3)n2)C1] | ¹H-NMR (400 MHz, DMSO-d6) δ 9.96 (s, 1H), 9.19 (d, J = 2.2 Hz, 1H), 8.70 (dd, J = 4.8, 1.6 Hz, 1H), 8.38 (dt, J = 8.2, 2.0 Hz, 1H), 8.05-7.90 (m, 1H), 7.57 (dd, J = 8.0, 4.8 Hz, 1H), 7.42-7.32 (m, 2H), 7.15 (s, 1H), 6.90-6.79 (m, 1H), 3.25-3.16 (m, 1H), 3.02-2.90 (m, 1H), 2.90-2.81 (m, 1H), 2.36-2.17 (m, 3H), 2.18-2.04 (m, 1H), 1.99-1.86 (m, 1H), 1.82-1.71 (m, 1H), 1.71-1.56 (m, 2H), 1.48 (h, J = 7.3 Hz, 2H), 0.86 (t, J = 7.3 Hz, 3H); LCMS (Method D): t_{R} 3.85 min, 100%, MS (ESI) 392.2 (M + H)⁺. |

### Example 9: Synthesis of 1-(3-(4-(cyclopropylamino)-6-(pyridin-3-yl)pyrimidin-2-yl)piperidin-1-yl)propan-1-one (00178)

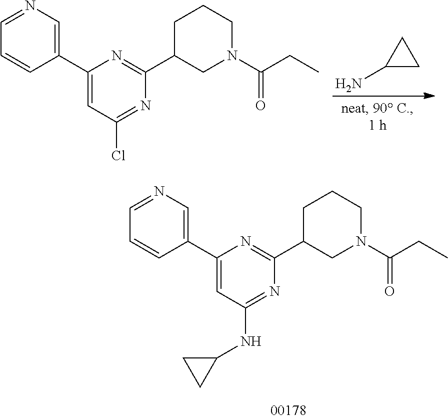

A microwave vial was charged with 1-(3-(4-chloro-6-(pyridin-3-yl)pyrimidin-2-yl)piperidin-1-yl)propan-1-one (25 mg, 0.08 mmol) and cyclopropylamine (1 mL, 14.19 mmol), capped and heated to 90° C. for 1 hour in a microwave. The mixture was concentrated and purified by reversed phase chromatography (method B) to afford 1-(3-(4-(cyclopropylamino)-6-(pyridin-3-yl)pyrimidin-2-yl)piperidin-1-yl)propan-1-one (10 mg, 36%) as a white solid. ¹H-NMR (400 MHz, DMSO-d6) mixture of rotamers δ 9.24 (s, 1H), 8.68 (d, J=4.7 Hz, 1H), 8.41 (s, 1H), 7.75 (s, 1H), 7.54 (dd, J=8.1, 5.0 Hz, 1H), 7.04 (s, 1H), 4.79-4.60 (m, 0.5H), 4.23 (s, 0.5H), 4.03 (d, J=13.2 Hz, 0.5H), 3.88 (d, J=13.5 Hz, 0.5H), 3.45 (s, 0.5H), 3.00 (t, J=12.8 Hz, 0.5H), 2.69 (d, J=55.7 Hz, 2.5H), 2.35 (q, J=7.4 Hz, 2H), 2.09 (d, J=12.3 Hz, 1H), 1.96-1.67 (m, 2H), 1.44 (dt, J=35.7, 12.0 Hz, 1H), 0.99 (dt, J=13.1, 7.4 Hz, 3H), 0.87-0.71 (m, 2H), 0.52 (p, J=4.9, 4.5 Hz, 2H)); LCMS (Method D): t_{R} 2.92 min, 100%, MS (ESI) 352.2 (M+H)⁺

The following compounds were prepared using procedures analogous to Example 9:

| Compound # | Structure and compound name | Analytical data |
|---|---|---|
| 00179 | 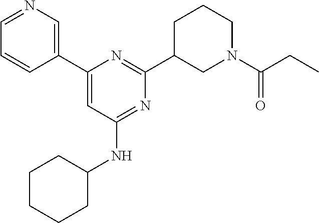 [SMILES: CCC(=O)N1CCCC(c2nc(NC3CCCCC3)cc(-c3cccnc3)n2)C1] 1-(3-(4-(cyclohexylamino)-6- | ¹H-NMR (400 MHz, DMSO-d₆) mixture of rotamers and tautomers δ 9.12 (s, 1H), 8.65 (d, J = 4.7 Hz, 1H), 8.30 (s, 1H), 7.52 (dd, J = 7.9, 5.0 Hz, 1H), 7.35 (s, 1H), 6.82 (s, 1H), 4.67 (d, J = 12.8 Hz, 0.5H), 4.17 (s, 0.5H), 4.07 − 4.00 (m, 0.5H), 3.87 (d, J = 13.8 Hz, 1H), 3.45 (t, J = 11.8 Hz, 0.5H), 3.01 (t, J = 12.9 Hz, 0.5H), 2.88 − 2.68 (m, 1.5H), 2.61 (d, J = 11.1 Hz, 0.5H), 2.35 (qd, J = 7.7, 2.4 Hz, 2H), 2.11 (d, J = 13.0 Hz, 1H), 1.99 − 1.10 (m, 35H), 1.05 − 0.92 (m, 3H); LCMS (Method D): t_{R} 3.45 min, 100%, MS (ESI) 394.2 (M + H)⁺. |
| | (pyridin-3-yl)pyrimidin-2- | |
| | yl)piperidin-1-yl)propan-1-one | |
| 00180 | 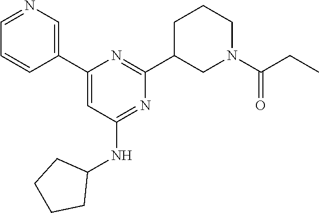 [SMILES: CCC(=O)N1CCCC(c2nc(NC3CCCC3)cc(-c3cccnc3)n2)C1] 1-(3-(4-(cyclopentylamino)-6- (pyridin-3-yl)pyrimidin-2- | ¹H-NMR (400 MHz, DMSO-d6) mixture of rotamers δ 9.13 (s, 1H), 8.66 (d, J = 4.8 Hz, 1H), 8.31 (s, 1H), 7.64 − 7.38 (m, 2H), 6.82 (s, 1H), 4.68 (d, J = 12.7 Hz, 0.5H), 4.24 (d, J = 39.4 Hz, 1H), 4.03 (d, J = 13.7 Hz, 0.5H), 3.87 (d, J = 13.4 Hz, 0.5H), 3.46 (t, J = 11.8 Hz, 0.5H), 3.01 (t, J = 12.9 Hz, 0.5H), 2.88 − 2.69 (m, 1H), 2.66 − 2.56 (m, 0.5H), 2.41 − 2.28 (m, 2H), 2.21 − 2.06 (m, 1H), 2.04 − 1.94 (m, 2H), 1.89 − 1.64 (m, 4H), 1.64 − 1.35 (m, 5H), 1.07 − 0.91 (m, 3H); LCMS (Method D): t_{R} 3.30 min, 100%, MS (ESI) 380.2 (M + H)⁺. |
| | yl)piperidin-1-yl)propan-1-one | |
| 00181 | 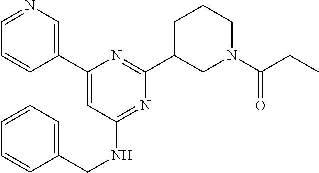 [SMILES: CCC(=O)N1CCCC(c2nc(NCc3ccccc3)cc(-c3cccnc3)n2)C1] 1-(3-(4-(benzylamino)-6-(pyridin-3- yl)pyrimidin-2-yl)piperidin-1- yl)propan-1-one | ¹H-NMR (400 MHz, DMSO-d6) mixture of rotamers δ 9.15 (s, 1H), 8.65 (d, J = 4.7 Hz, 1H), 8.33 (d, J = 7.9 Hz, 1H), 8.02 (dd, J = 14.0, 7.8 Hz, 1H), 7.57 − 7.48 (m, 1H), 7.41 − 7.30 (m, 4H), 7.24 (t, J = 7.2 Hz, 1H), 6.91 (d, J = 4.8 Hz, 1H), 4.74 − 4.50 (m, 2H), 4.18 (d, J = 12.8 Hz, 0.5H), 3.99 (d, J = 13.5 Hz, 0.5H), 3.86 (d, J = 13.6 Hz, 0.5H), 3.41 (t, J = 11.9 Hz, 0.5H), 2.99 (t, J = 12.3 Hz, 0.5H), 2.85 − 2.58 (m, 2H), 2.39 − 2.26 (m, 2H), 2.10 (d, J = 12.5 Hz, 1H), 1.92 − 1.66 (m, 2H), 1.59 − 1.32 (m, 1H), 1.06 − 0.90 (m, 3H); LCMS (Method D): t_{R} 3.25 min, |
| | | 100%, MS (ESI) 402.2 (M + H)⁺. |
| 00182 | 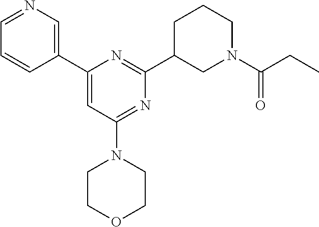 [SMILES: CCC(=O)N1CCCC(c2nc(-c3cccnc3)cc(N3CCOCC3)n2)C1] 1-(3-(4-morpholino-6-(pyridin-3- yl)pyrimidin-2-yl)piperidin-1- yl)propan-1-one | ¹H-NMR (400 MHz, DMSO-d6) mixture of rotamers δ 9.33 (s, 1H), 8.67 (d, J = 4.6 Hz, 1H), 8.50 (dt, J = 7.9, 2.0 Hz, 1H), 7.62 − 7.46 (m, 1H), 7.31 (d, J = 3.9 Hz, 1H), 4.66 (d, J = 12.8 Hz, 0.5H), 4.18 (d, J = 12.9 Hz, 0.5H), 4.03 (d, J = 13.6 Hz, 0.5H), 3.86 (d, J = 13.8 Hz, 0.5H), 3.71 (dd, J = 5.6, 3.1 Hz, 8H), 3.49 (dd, J = 13.5, 10.0 Hz, 0.5H), 3.03 (t, J = 12.6 Hz, 0.5H), 2.90 − 2.75 (m, 1.5H), 2.73 − 2.61 (m, 0.5H), 2.35 (q, J = 7.3 Hz, 2H), 2.21 − 2.05 (m, 1H), 1.79 (ddd, J = 38.2, 15.8, 12.1 Hz, 2H), 1.47 (dd, J = 34.9, 12.5 Hz, 1H), 0.98 (dt, J = 14.3, 7.4 Hz, 3H); LCMS (Method D): t_{R} 2.89 min, 100%, MS (ESI) 382.2 (M + H)⁺. |
| 00183 | 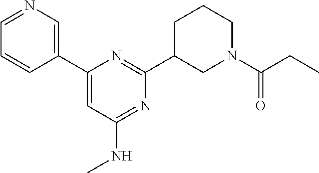 [SMILES: CCC(=O)N1CCCC(c2nc(NC)cc(-c3cccnc3)n2)C1] 1-(3-(4-(methylamino)-6-(pyridin-3- yl)pyrimidin-2-yl)piperidin-1- yl)propan-1-one | ¹H-NMR (400 MHz, DMSO-d6) mixture of rotamers δ 9.19 (s, 1H), 8.66 (d, J = 4.7 Hz, 1H), 8.37 (s, 1H), 7.65 − 7.47 (m, 1H), 7.40 (s, 1H), 6.86 (s, 1H), 4.68 (d, J = 12.7 Hz, 0.5H), 4.20 (s, 0.5H), 4.02 (d, J = 13.4 Hz, 0.5H), 3.88 (d, J = 13.4 Hz, 0.5H), 3.48 (s, 0.5H), 3.02 (t, J = 12.8 Hz, 0.5H), 2.96 − 2.70 (m, 4.5H), 2.62 (s, 0.5H), 2.41 − 2.26 (m, 2H), 2.10 (d, J = 14.7 Hz, 1H), 1.95 − 1.68 (m, 2H), 1.62 − 1.34 (m, 1H), 1.09 − 0.90 (m, 3H); LCMS (Method D): t_{R} 2.74 min, 100%, MS (ESI) 326.2 (M + H)⁺. |

### Example 10: Synthesis of 1-(3-(4-((3-fluorophenyl)amino)-6-(tetrahydro-2H-pyran-4-yl)pyrimidin-2-yl)piperidin-1-yl)propan-1-one (00184)

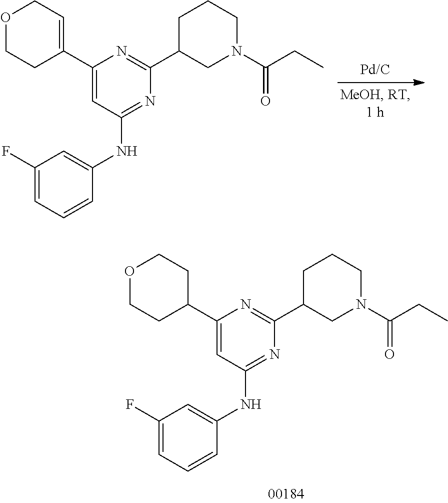 [SMILES: CCC(=O)N1CCCC(c2nc(Nc3cccc(F)c3)cc(C3=CCOCC3)n2)C1 ; CCC(=O)N1CCCC(c2nc(Nc3cccc(F)c3)cc(C3CCOCC3)n2)C1]

Under argon atmosphere, 1-(3-(4-(3,6-dihydro-2H-pyran-4-yl)-6-((3-fluorophenyl)amino)pyrimidin-2-yl)piperidin-1-yl)propan-1-one (10 mg, 0.02 mmol) was dissolved in methanol (2 mL) and a catalytic amount of 10% palladium on activated carbon was added. Next, hydrogen atmosphere was introduced and the mixture was stirred at room temperature for 1 hour. The mixture was flushed with nitrogen, filtered over Celite and the filtrate was lyophilized to afford 1-(3-(4-((3-fluorophenyl)amino)-6-(tetrahydro-2H-pyran-4-yl)pyrimidin-2-yl)piperidin-1-yl)propan-1-one (7 mg, 66%) as a white solid. ¹H-NMR (400 MHz, DMSO-d6) mixture of rotamers δ 9.74 (d, J=10.5 Hz, 1H), 7.85 (dd, J=19.1, 12.1 Hz, 1H), 7.41-7.29 (m, 2H), 6.86-6.75 (m, 1H), 6.51 (s, 1H), 4.71 (d, J=12.7 Hz, 0.5H), 4.18 (d, J=13.1 Hz, 0.5H), 4.14-4.05 (m, 0.5H), 4.00-3.82 (m, 2.5H), 3.51-3.37 (m, 2.5H), 3.00 (t, J=12.8 Hz, 0.5H), 2.90-2.58 (m, 3H), 2.43-2.29 (m, 2H), 2.21-2.07 (m, 1H), 1.92-1.61 (m, 6H), 1.60-1.36 (m, 1H), 0.99 (q, J=7.3 Hz, 3H); LCMS (Method D): t_{R} 3.38 min, 100%, MS (ESI) 413.2 (M+H)⁺

The following compounds were prepared using procedures analogous to Example 10:

| Compound # | Structure and compound name | Analytical data |
|---|---|---|
| 00185 | 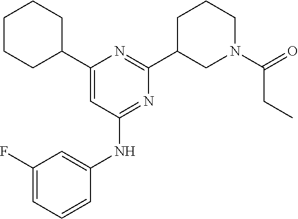 [SMILES: CCC(=O)N1CCCC(c2nc(Nc3cccc(F)c3)cc(C3CCCCC3)n2)C1] | ¹H-NMR (400 MHz, DMSO-d6) mixture of rotamers δ 9.69 (s, 1H), 7.95 − 7.74 (m, 1H), 7.34 (t, J = 7.0 Hz, 2H), 6.79 (s, 1H), 6.49 (s, 1H), 4.70 (d, J = 12.2 Hz, 0.5H), 4.16 (d, J = 12.8 Hz, 1.5), 4.06 (d, J = 13.7 Hz, 0.5H), 3.88 (d, J = 13.3 Hz, 0.5H), 3.45 − 3.37 (m, 0.5H), 3.00 (t, J = 12.8 Hz, 0.5H), 2.89 − 2.59 (m, 2H), 2.40 − 2.29 (m, 2H), 2.13 (d, J = 13.2 Hz, 1H), 1.91 -1.66 (m, 7H), 1.58- 1.17 (m, 7H), 0.99 (q, J = 7.5 Hz, 3H); LCMS (Method D): tR 4.05 min, 100%, MS (ESI) 411.2 (M + H)⁺. |
| | 1-(3-(4-cyclohexyl-6-((3- | |
| | fluorophenyl)amino)pyrimidin-2- | |
| | yl)piperidin-1-yl)propan-1-one | |
| 00186 | 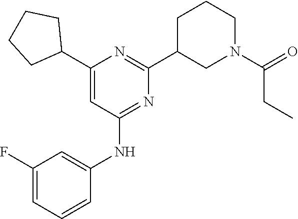 [SMILES: CCC(=O)N1CCCC(c2nc(Nc3cccc(F)c3)cc(C3CCCC3)n2)C1] 1-(3-(4-cyclopentyl-6-((3- | ¹H-NMR (400 MHz, DMSO-d6) mixture of rotamers δ 9.67 (d, J = 11.0 Hz, 1H), 7.85 (dd, J = 20.1, 12.2 Hz, 1H), 7.42 − 7.27 (m, 2H), 6.86 − 6.71 (m, 1H), 6.52 (d, J = 2.2 Hz, 1H), 4.70 (d, J = 12.6 Hz, 0.5H), 4.17 (d, J = 13.0 Hz, 0.5H), 4.08 (d, J = 12.6 Hz, 0.5H), 3.88 (d, J = 13.4 Hz, 0.5H), 3.39 (dd, J = 13.4, 9.9 Hz, 0.5H), 3.05 − 2.92 (m, 1.5H), 2.87 − 2.59 (m, 2H), 2.41 − 2.27 (m, 2H), 2.14 (d, J = 13.1 Hz, 1H), 2.02 − 1.90 (m, 2H), 1.86 − 1.36 (m, 11H), 0.99 (q, J = 7.4 Hz, 3H); LCMS (Method D): t_{R} 3.92 min, 100%, MS (ESI) 397.2 (M + H)⁺. |
| | fluorophenyl)amino)pyrimidin-2- | |
| | yl)piperidin-1-yl)propan-1-one | |

### Example 11: Synthesis of 1-(3-(4-((3-fluorophenyl)(methyl)amino)-6-(pyridin-3-yl)pyrimidin-2-yl)piperidin-1-yl)propan-1-one (00187)

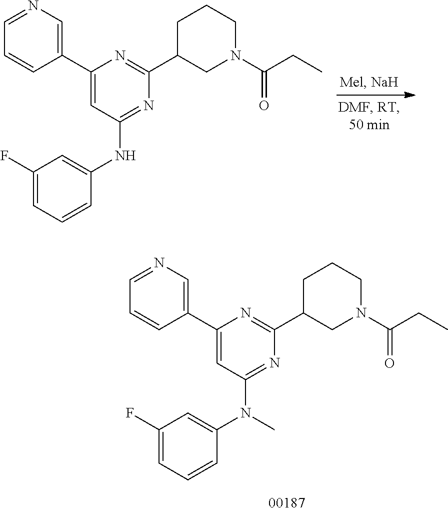 [SMILES: CCC(=O)N1CCCC(c2nc(Nc3cccc(F)c3)cc(-c3cccnc3)n2)C1 ; CCC(=O)N1CCCC(c2nc(-c3cccnc3)cc(N(C)c3cccc(F)c3)n2)C1]

Under nitrogen atmosphere, sodium hydride (60% in mineral oil, 1.78 mg, 0.04 mmol) was added at room temperature to a solution of 1-(3-(4-((3-fluorophenyl)amino)-6-(pyridin-3-yl)pyrimidin-2-yl)piperidin-1-yl)propan-1-one (15 mg, 0.04 mmol) in N,N-dimethylformamide (0.5 mL) and the mixture was stirred for 10 minutes. Next, iodomethane (2.31 μL, 0.04 mmol) was added and the mixture was stirred at room temperature for 40 minutes. The mixture was diluted with water (0.3 mL) and purified by reversed phase chromatography (method B) to afford 1-(3-(4-((3-fluorophenyl)(methyl)amino)-6-(pyridin-3-yl)pyrimidin-2-yl)piperidin-1-yl)propan-1-one (7 mg, 45%) as a white solid. ¹H-NMR (400 MHz, DMSO-d6) mixture of rotamers δ 9.22-9.10 (m, 1H), 8.71-8.60 (m, 1H), 8.33 (t, J=8.6 Hz, 1H), 7.59-7.43 (m, 2H), 7.41-7.32 (m, 1H), 7.28 (d, J=8.2 Hz, 1H), 7.18 (td, J=8.5, 2.6 Hz, 1H), 6.95 (d, J=19.4 Hz, 1H), 4.68 (d, J=12.5 Hz, 0.5H), 4.17 (d, J=12.9 Hz, 0.5H), 4.05 (d, J=13.9 Hz, 0.5H), 3.86 (d, J=13.1 Hz, 0.5H), 3.51 (s, 3H), 3.44 (dd, J=13.6, 10.0 Hz, 0.5H), 3.03 (t, J=12.6 Hz, 0.5H), 2.91-2.64 (m, 2H), 2.40-2.23 (m, 2H), 2.21-2.07 (m, 1H), 1.94-1.65 (m, 2H), 1.59-1.32 (m, 1H), 1.05-0.88 (m, 3H); LCMS (Method D): t_{R} 3.47 min, 100%, MS (ESI) 420.1 (M+H)⁺.

The following compounds were prepared using procedures analogous to Example 11:

| Compound # | Structure and compound name | Analytical data |
|---|---|---|
| 00188 | 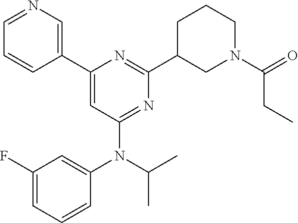 [SMILES: CCC(=O)N1CCCC(c2nc(-c3cccnc3)cc(N(c3cccc(F)c3)C(C)C)n2)C1] 1-(3-(4-((3- fluorophenyl)(isopropyl)amino)-6- (pyridin-3-yl)pyrimidin-2- | ¹H-NMR (400 MHz, DMSO-d₆) mixture of rotamers δ 9.05 − 8.94 (m, 1H), 8.67 − 8.57 (m, 1H), 8.21 − 8.10 (m, 1H), 7.67 − 7.56 (m, 1H), 7.50 − 7.42 (m, 1H), 7.40 − 7.31 (m, 1H), 7.27 − 7.18 (m, 1H), 7.17 − 7.09 (m, 1H), 6.35 − 6.19 (m, 1H), 5.31 − 5.13 (m, 1H), 4.76 − 4.65 (m, 0.5H), 4.24 − 4.13 (m, 0.5H), 4.11 − 4.00 (m, 0.5H), 3.93 3.82 (m, 0.5H), 3.52 − 3.42 (m, 0.5H), 3.10 − 2.98 (m, 0.5H), 2.91 − 2.64 (m, 2H), 2.41 − 2.28 (m, 2H), 2.25 − 2.11 (m, 1H), 1.95 − 1.69 (m, 2H), 1.62 − 1.36 (m, 1H), 1.15 (d, J = 6.5 Hz, 6H), 1.06 − 0.93 (m, 3H); LCMS (Method D): t_{R} 3.82 min, 100%, MS (ESI) 448.2 (M + H)⁺. |
| | yl)piperidin-1-yl)propan-1-one | |
| 00189 | 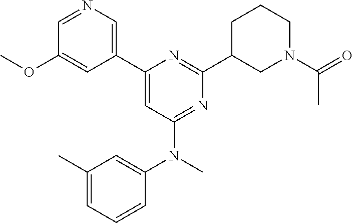 [SMILES: COc1cncc(-c2cc(N(C)c3cccc(C)c3)nc(C3CCCN(C(C)=O)C3)n2)c1] 1-(3-(4-(5-methoxypyridin-3-yl)-6- (methyl(m-tolyl)amino)pyrimidin-2- yl)piperidin-1-yl)ethan-1-one | ¹H-NMR (400 MHz, DMSO-d₆) mixture of rotamers δ 8.63 (dd, J = 10.8, 1.8 Hz, 1H), 8.37 (t, J = 2.9 Hz, 1H), 7.84 − 7.75 (m, 1H), 7.38 (t, J = 7.8 Hz, 1H), 7.24 (s, 1H), 7.18 (dd, J = 13.5, 7.7 Hz, 2H), 6.77 (d, J = 12.0 Hz, 1H), 4.63 (d, J = 14.0 Hz, 0.5H), 4.11 (d, J = 13.0 Hz, 0.5H), 4.01 (d, J = 12.8 Hz, 0.5H), 3.88 (s, 3H), 3.81 (d, J = 13.7 Hz, 0.5H), 3.54 (dd, J = 13.5, 9.7 Hz, 0.5H), 3.49 (s, 3H), 3.14 − 3.01 (m, 0.5H), 2.98 − 2.80 (m, 1.5H), 2.78 − 2.62 (m, 0.5H), 2.36 (s, 3H), 2.15 (d, J = 11.8 Hz, 1H), 2.01 (d, J = 10.9 Hz, 3H), 1.95 − 1.67 (m, 2H), 1.65 − 1.34 (m, 1H); LCMS (Method D): t_{R} 3.56 min, 100%, MS (ESI) 432.2 (M + H)⁺. |

### Example 12: Synthesis of 1-(3-(4-(3-fluorophenoxy)-6-(pyridin-3-yl)pyrimidin-2-yl)piperidin-1-yl)propan-1-one (00191)

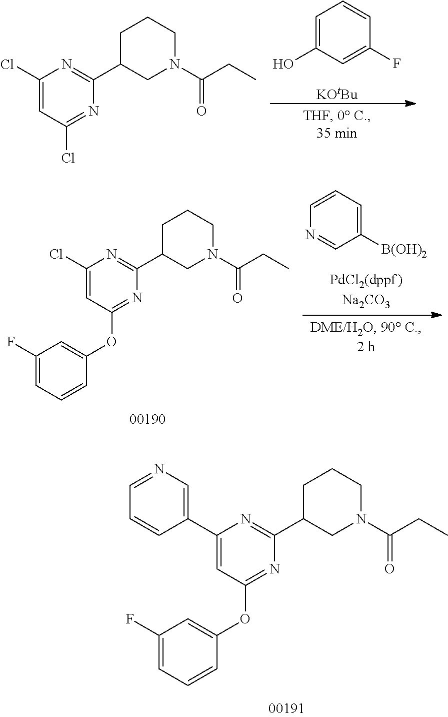

A solution of 3-fluorophenol (14.78 mg, 0.13 mmol) in tetrahydrofuran (2 mL) was cooled to 0° C. Next, potassium tert-butoxide (20.25 mg, 0.18 mmol) was added and the mixture was stirred for 5 minutes. This mixture was added to a ice-cooled solution of 1-(3-(4,6-dichloropyrimidin-2-yl)piperidin-1-yl)propan-1-one (40 mg, 0.14 mmol) in tetrahydrofuran (2 mL) and stirred at 0° C. for 30 minutes. The mixture was concentrated in vacuo and the residue was purified by reversed phase chromatography (method A) to afford 1-(3-(4-chloro-6-(3-fluorophenoxy)pyrimidin-2-yl)piperidin-1-yl)propan-1-one (42 mg, 83%) as a colorless gum. ¹H-NMR (400 MHz, DMSO-d6) mixture of rotamers δ 7.52 (q, J=7.9 Hz, 1H), 7.30-7.11 (m, 4H), 4.54 (d, J=8.3 Hz, 0.5H), 4.02 (d, J=13.0 Hz, 0.5H), 3.93-3.85 (m, 0.5H), 3.79 (d, J=13.6 Hz, 0.5H), 2.95 (t, J=12.2 Hz, 0.5H), 2.88-2.74 (m, 1H), 2.71-2.59 (m, 1H), 2.35-2.21 (m, 2H), 2.09-1.89 (m, 1H), 1.77-1.53 (m, 2H), 1.52-1.26 (m, 1H), 1.02-0.88 (m, 3H); LCMS (Method D): t_{R} 3.68 min, 100%, MS (ESI) 364.1 (M+H)⁺. Under argon atmosphere, a microwave vial was charged with 1-(3-(4-chloro-6-(3-fluorophenoxy)pyrimidin-2-yl)piperidin-1-yl)propan-1-one (36 mg, 0.10 mmol), pyridine-3-boronic acid (18.24 mg, 0.15 mmol), PdCl₂(dppf) (3.62 mg, 4.95 μmol; CAS Number 72287-26-4) and sodium carbonate (20.98 mg, 0.20 mmol) in a mixture of 1,2-dimethoxyethane (3 mL) and water (1 mL). The mixture was heated in a microwave at 90° C. for 2 hours, poured into water and extracted with ethyl acetate twice. The combined organic layers were washed with brine once, dried with sodium sulfate and concentrated in vacuo. The residue was purified by reversed phase chromatography (method B) to afford 1-(3-(4-(3-fluorophenoxy)-6-(pyridin-3-yl)pyrimidin-2-yl)piperidin-1-yl)propan-1-one (28 mg, 70%) as a white solid. ¹H-NMR (400 MHz, DMSO-d6) mixture of rotamers δ 9.40 (s, 1H), 8.84-8.68 (m, 1H), 8.57 (d, J=7.9 Hz, 1H), 7.77-7.47 (m, 3H), 7.35-7.23 (m, 1H), 7.23-7.03 (m, 2H), 4.57 (d, 0.5H), 4.03 (dd, J=32.4, 13.1 Hz, 1H), 3.80 (d, J=13.5 Hz, 0.5H), 3.42 (m, 0.5H), 3.01 (t, J=12.6 Hz, 0.51H), 2.96-2.69 (m, 2H), 2.38-2.19 (m, 2H), 2.08 (s, 1H), 1.92-1.60 (m, 2H), 1.60-1.32 (m, 1H), 1.07-0.82 (m, 3H); LCMS (Method D): t_{R} 3.48 min, 100%, MS (ESI) 407.2 (M+H)⁺.

The following compound was prepared using procedures analogous to Example 12:

| Compound # | Structure and compound name | Analytical data |
|---|---|---|
| 00192 | 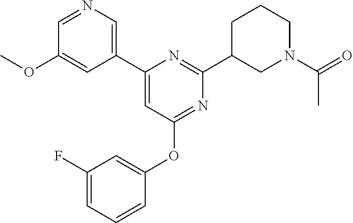 [SMILES: COc1cncc(-c2cc(Oc3cccc(F)c3)nc(C3CCCN(C(C)=O)C3)n2)c1] | ¹H-NMR (400 MHz, DMSO-d₆) mixture of rotamers δ 9.01 (dd, J = 4.6, 1.7 Hz, 1H), 8.47 (t, J = 2.7 Hz, 1H), 8.10 (d, J = 2.4 Hz, 1H), 7.78 (d, J = 7.1 Hz, 1H), 7.57 − 7.46 (m, 1H), 7.32 − 7.22 (m, 1H), 7.21 − 7.11 (m, 2H), 4.54 − 4.48 (m, 0.5H), 3.94 (s, 4H), 3.74 (d, J = 13.4 Hz, 0.5H), 3.47 (dd, J = 13.6, 9.5 Hz, 0.5H), 3.05 (t, J = 11.9 Hz, 0.5H), 2.97 − 2.69 (m, 2H), 2.15 − 2.02 (m, 1H), 1.99 (s, 1.5H), 1.93 (s, 1.5H), 1.86 − 1.59 (m, 2H), 1.58 − 1.31 (m, 1H); LCMS (Method D): t_{R} 3.41 min, 100%, MS (ESI) 423.1 (M + H)⁺. |
| | 1-(3-(4-(3-fluorophenoxy)-6-(5- methoxypyridin-3-yl)pyrimidin-2- yl)piperidin-1-yl)ethan-1-one | |

### Example 13: Synthesis of 1-(3-(4-(3-fluorobenzyl)-6-(pyridin-3-yl)pyrimidin-2-yl)piperidin-1-yl)propan-1-one (00193)

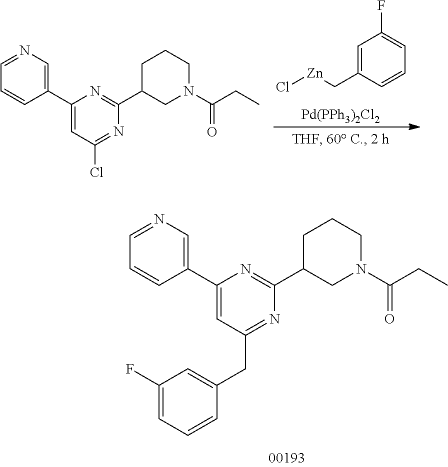

Under argon atmosphere, a microwave vial was charged with 1-(3-(4-chloro-6-(pyridin-3-yl)pyrimidin-2-yl)piperidin-1-yl)propan-1-one (30 mg, 0.09 mmol) and bis(triphenylphosphine) palladium(II) chloride (3.18 mg, 4.53 μmol) in tetrahydrofuran (4 mL). Next, a 0.5M 3-fluorobenzylzinc chloride solution in tetrahydrofuran (0.19 mL, 0.1 mmol) was added and the mixture was heated in a microwave at 60° C. for 2 hours. The mixture was poured into water and extracted with ethyl acetate twice. The combined organic layers were washed with brine, dried with sodium sulfate, concentrated in vacuo and the residue was purified by reversed phase chromatography (method B) to afford 1-(3-(4-(3-fluorobenzyl)-6-(pyridin-3-yl)pyrimidin-2-yl)piperidin-1-yl)propan-1-one (17 mg, 44%) as a light brown gum. ¹H-NMR (400 MHz, DMSO-d6) mixture of rotamers δ 9.35 (s, 1H), 8.73 (d, J=4.6 Hz, 1H), 8.52 (d, J=8.0 Hz, 1H), 8.03 (d, J=5.0 Hz, 1H), 7.58 (t, J=6.4 Hz, 1H), 7.37 (q, J=7.4 Hz, 1H), 7.23 (t, J=10.3 Hz, 2H), 7.07 (t, J=8.9 Hz, 1H), 4.68 (d, J=10.1 Hz, 0.5H), 4.16 (s, 2H), 4.15-4.00 (m, 1H), 3.87 (d, J=13.4 Hz, 0.5H), 3.56 (dd, J=13.5, 9.7 Hz, 0.5H), 3.14-2.98 (m, 1H), 2.98-2.81 (m, 1H), 2.43-2.26 (m, 2H), 2.22-2.08 (m, 1H), 2.03-1.66 (m, 2H), 1.62-1.38 (m, 1H), 1.06-0.86 (m, 3H); LCMS (Method D): t_{R} 3.42 min, 100%, MS (ESI) 405.2 (M+H)⁺.

The following compound was prepared using procedures analogous to Example 13:

| Compound # | Structure and compound name | Analytical data |
|---|---|---|
| 00194 | 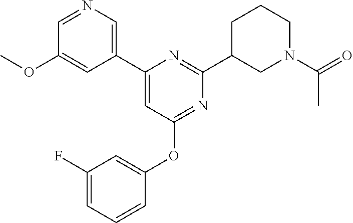 [SMILES: COc1cncc(-c2cc(Oc3cccc(F)c3)nc(C3CCCN(C(C)=O)C3)n2)c1] | ¹H-NMR (400 MHz, DMSO-d₆) mixture of rotamers δ 8.99 − 8.93 (m, 1H), 8.46 (t, J = 2.8 Hz, 1H), 8.09 − 8.02 (m, 2H), 7.41 − 7.31 (m, 1H), 7.26 − 7.17 (m, 2H), 7.11 − 7.02 (m, 1H), 4.68 − 4.58 (m, 0.5H), 4.20 − 4.12 (m, 2H), 4.12 -3.97 (m, 1H), 3.94 (s, 3H), 3.86 − 3.77 (m, 0.5H), 3.67 -3.57 (m, 0.5H), 3.17 − 2.84 (m, 2.5H), 2.21 − 2.09 (m, 1H), 2.02 (s, 1.5H), 1.98 (s, 1.5H), 1.96 − 1.39 (m, 3H); LCMS (Method D): t_{R} 3.33 min, 99%, MS (ESI) 421.2 (M + H)⁺. |
| | 1-(3-(4-((3- | |
| | fluorophenyl)(isopropyl)amino)-6- | |
| | (pyridin-3-yl)pyrimidin-2- | |
| | yl)piperidin-1-yl)propan-1-one | |

### Example 14: Synthesis of 1-(3-(6′-((3-fluorophenyl)amino)-[2,4′-bipyrimidin]-2′-yl)piperidin-1-yl)propan-1-one (00195)

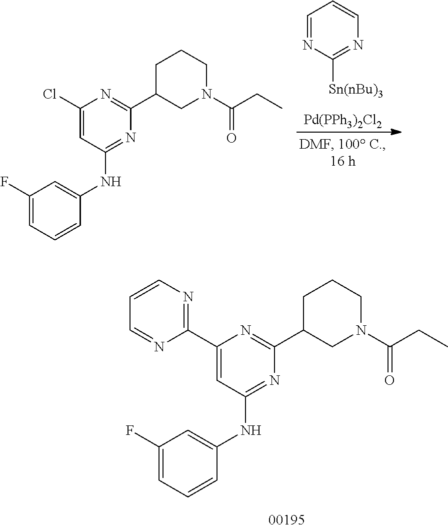

Under argon atmosphere, 1-(3-(4-chloro-6-((3-fluorophenyl)amino)pyrimidin-2-yl)piperidin-1-yl)propan-1-one (30 mg, 0.08 mmol), 2-(tributylstannyl)-pyrimidine (33.6 mg, 0.09 mmol) and bis(triphenylphosphine)palladium(II) dichloride (5.80 mg, 8.27 μmol) were dissolved in N,N-dimethylformamide (2 mL) and heated at 100° C. for 16 hours. The mixture was partitioned between water and ethyl acetate and the aqueous layer was extracted with ethyl acetate twice. The combined organic layers were dried with sodium sulfate, concentrated in vacuo and the residue was purified by reversed phase chromatography (method B) to afford 1-(3-(6′-((3-fluorophenyl)amino)-[2,4′-bipyrimidin]-2′-yl)piperidin-1-yl)propan-1-one (9 mg, 25%) as a light yellow solid. ¹H-NMR (400 MHz, DMSO-d6) mixture of rotamers δ 10.08 (d, J=8.7 Hz, 1H), 9.02 (d, J=4.8 Hz, 2H), 7.91 (t, J=13.8 Hz, 1H), 7.73 (s, 1H), 7.63 (t, J=4.8 Hz, 1H), 7.48-7.34 (m, 2H), 6.85 (t, J=8.4 Hz, 1H), 4.82 (d, J=8.6 Hz, 0.5H), 4.15 (dd, J=31.7, 13.2 Hz, 1H), 3.93 (d, J=13.7 Hz, 0.5H), 3.60-3.51 (m, 0.5H), 3.09-2.92 (m, 1H), 2.89-2.75 (m, 1.5H), 2.45-2.31 (m, 2H), 2.28-2.15 (m, 1H), 2.03-1.68 (m, 2H), 1.65-1.39 (m, 1H), 1.05-0.94 (m, 3H); LCMS (Method D): t_{R} 3.13 min, 100%, MS (ESI) 407.2 (M+H)⁺.

The following compounds were prepared using procedures analogous to Example 14:

| Compound # | Structure and compound name | Analytical data |
|---|---|---|
| 00196 | 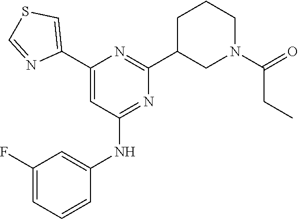 [SMILES: CCC(=O)N1CCCC(c2nc(Nc3cccc(F)c3)cc(-c3cscn3)n2)C1] 1-(3-(4-((3-fluorophenyl)amino)-6- (thiazol-4-yl)pyrimidin-2- | ¹H-NMR (400 MHz, DMSO-d6) mixture of rotamers δ 9.97 (d, J = 10.1 Hz, 1H), 9.26 (d, J = 2.0 Hz, 1H), 8.45 (dd, J = 7.1, 2.1 Hz, 1H), 7.90 (dd, J = 21.5, 12.2 Hz, 1H), 7.53 − 7.27 (m, 3H), 6.92 − 6.73 (m, 1H), 4.72 (s, 0.5H), 4.24 (dd, J = 30.0, 13.3 Hz, 1H), 3.90 (d, J = 13.6 Hz, 0.5H), 3.44 (dd, J = 13.5, 10.3 Hz,0.51H), 3.05 (t, J = 12.5 Hz, 0.5H), 2.97 − 2.83 (m, 1H), 2.83 − 2.70 (m, 1H), 2.46 − 2.31 (m, 2H), 2.28 − 2.15 (m, 1H), 1.98 − 1.73 (m, 2H), 1.67 − 1.39 (m, 1H), 1.05 − 0.94 (m, 3H); LCMS (Method D): t_{R} 3.52 min, 100%, MS (ESI) 412.1 (M + H)⁺. |
| | yl)piperidin-1-yl)propan-1-one | |
| 00197 | 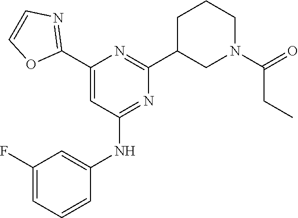 1-(3-(4-((3-fluorophenyl)amino)-6- | ¹H-NMR (400 MHz, DMSO-d6) mixture of rotamers δ 10.11 (d, J = 9.5 Hz, 1H), 8.36 (d, J = 3.9 Hz, 1H), 7.92 − 7.79 (m, 1H), 7.67 − 7.49 (m, 3H), 7.44 − 7.34 (m, 3H), 6.93 − 6.82 (m, 1H), 4.83 − 4.64 (m, 0.5H), 4.23 − 4.12 (m, 0.5H), 4.12 − 4.02 (m, 0.5H), 3.92 − 3.82 (m, 0.5H), 3.58 − 3.46 (m, 0.5H), 3.12 − 3.01 (m, 0.5H), 3.01 − 2.91 (m, 0.5H), 2.87 − 2.70 (m, 1.5H), 2.26 − 2.13 (m, 0.5H), 2.05 (d, J = 8.0 Hz, 3H), 1.96 − 1.67 (m, 2H), 1.66 − 1.38 (m, 1H); LCMS (Method B): t_{R} 3.13 min, 97%, MS (ESI) 382.1 (M + H)⁺. |
| | (oxazol-2-yl)pyrimidin-2- | |
| | yl)piperidin-1-yl)ethan-1-one | |

The following further compounds were prepared using procedures analogous to Example 14:

| Compound # | Structure and compound name | Analytical data |
|---|---|---|
| 00198 | 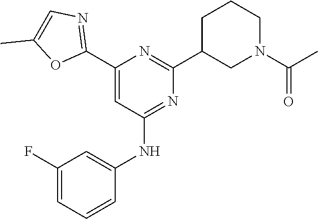 [SMILES: CC(=O)N1CCCC(c2nc(Nc3cccc(F)c3)cc(-c3ncc(C)o3)n2)C1] 1-(3-(4-((3-fluorophenyl)amino)-6- | ¹H-NMR (400 MHz, DMSO-d6) mixture of rotamers δ 9.97 (d, J = 9.6 Hz, 1H), 7.93 − 7.80 (m, 1H), 7.75 − 7.66 (m, 1H), 7.66 − 7.51 (m, 2H), 7.43 − 7.31 (m, 2H), 6.95 − 6.89 (m, 1H), 6.89 − 6.77 (m, 1H), 4.78 − 4.62 (m, 0.5H), 4.25 − 4.10 (m, 0.5H), 4.11 − 4.02 (m, 0.5H), 3.99 − 3.76 (m, 0.5H), 3.55 − 3.44 (m, 0.5H), 3.16 − 2.99 (m, 0.5H), 2.96 − 2.85 (m, 0.5H), 2.84 − 2.68 (m, 1.5H), 2.53 (s, 3H), 2.23 − 2.11 (m, 1H), 2.09 − 1.97 (m, 3H), 1.91 − 1.66 (m, 2H), 1.66 − 1.36 (m, 1H); LCMS (Method B): t_{R} 3.13 min, 100%, MS (ESI) 396.1 (M + H)⁺. |
| | (5-methyloxazol-2-yl)pyrimidin-2- | |
| | yl)piperidin-1-yl)ethan-1-one | |
| 00199 | 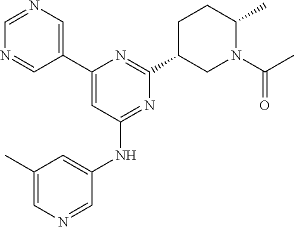 [SMILES: CC(=O)N1C[C@H](c2nc(Nc3cncc(C)c3)cc(-c3cncnc3)n2)CC[C@@H]1C] | ¹H NMR (400 MHz, DMSO-d6) mixture of rotamers δ 10.04 (s, 1H), 9.44 − 9.29 (m, 3H), 8.75 − 8.64 (m, 1H), 8.20 − 8.06 (m, 2H), 7.28 − 7.21 (m, 1H), 4.87 − 4.78 (m, 0.5H), 4.78 − 4.70 (m, 0.5H), 4.26 − 4.16 (m, 0.5H), 4.08 − 3.98 (m, 0.5H), 3.49 − 3.40 (m, 0.5H), 2.97 − 2.83 (m, 1H), 2.79 − 2.69 (m, 0.5H), 2.38 − 2.27 (m, 3H), 2.12 − 1.62 (m, 7H), 1.27 (d, J = 6.8 Hz, 1.5H), 1.15 (d, J = 6.9 Hz, 1.5H).; LCMS (Method D): t_{R} 2.99 min, 95%, MS (ESI) 404.1 (M + H)⁺. |
| | (+/−)-cis-1-(2-methyl-5-(6-((5- | |
| | methylpyridin-3-yl)amino)-[4,5′- | |
| | bipyrimidin]-2-yl)piperidin-1- | |
| | yl)ethan-1-one | |

### Example 15: Synthesis of 3-(4-((3-fluorophenyl)amino)-6-(pyridin-3-yl)pyrimidin-2-yl)-N-methylpiperidine-1-carboxamide (00200)

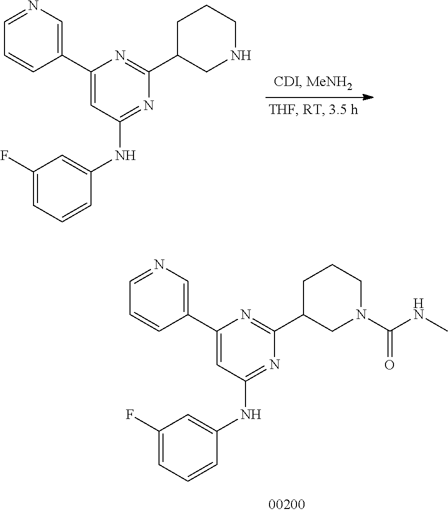 [SMILES: Fc1cccc(Nc2cc(-c3cccnc3)nc(C3CCCNC3)n2)c1 ; CNC(=O)N1CCCC(c2nc(Nc3cccc(F)c3)cc(-c3cccnc3)n2)C1]

To a solution of 1,1′-carbonyldiimidazole (11.60 mg, 0.07 mmol) in tetrahydrofuran (1 mL) was added 2M methylamine in tetrahydrofuran (0.04 ml, 0.07 mmol) and the mixture was stirred at room temperature for 30 minutes. Next, N-(3-fluorophenyl)-2-(piperidin-3-yl)-6-(pyridin-3-yl)pyrimidin-4-amine (25 mg, 0.07 mmol) in tetrahydrofuran (1 mL) was added and the mixture was stirred at room temperature for 3 hours. The mixture was concentrated in vacuo and purified by reversed phase chromatography (method A) to afford 3-(4-((3-fluorophenyl)amino)-6-(pyridin-3-yl)pyrimidin-2-yl)-N-methylpiperidine-1-carboxamide (7 mg, 23%) as a white solid. ¹H-NMR (400 MHz, DMSO-d6) mixture of rotamers δ 10.01 (s, 1H), 9.22 (d, J=2.2 Hz, 1H), 8.71 (d, J=4.9 Hz, 1H), 8.40 (dt, J=8.2, 2.1 Hz, 1H), 7.88 (dt, J=12.1, 2.3 Hz, 1H), 7.58 (dd, J=8.0, 4.8 Hz, 1H), 7.48-7.32 (m, 2H), 7.19 (s, 1H), 6.84 (td, J=8.3, 2.6 Hz, 1H), 6.52-6.40 (m, 1H), 4.29 (dd, J=12.7, 3.6 Hz, 1H), 3.95 (d, J=13.1 Hz, 1H), 3.04 (dd, J=13.0, 10.8 Hz, 1H), 2.88-2.65 (m, 2H), 2.56 (d, J=4.2 Hz, 3H), 2.17 (d, J=13.0 Hz, 1H), 1.87-1.67 (m, 2H), 1.59-1.42 (m, 1H); LCMS (Method D): t_{R} 3.11 min, 100%, MS (ESI) 407.2 (M+H)⁺.

### Example 16: Synthesis 1-(2-(4-((3-fluorophenyl)amino)-6-(pyridin-3-yl)pyrimidin-2-yl)morpholino)ethan-1-one (00201)

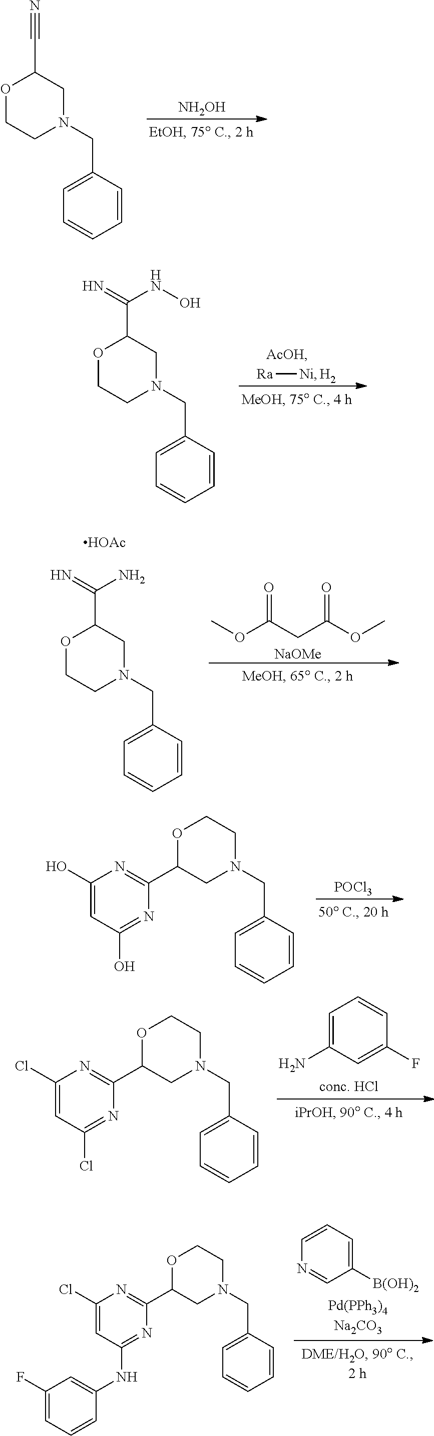

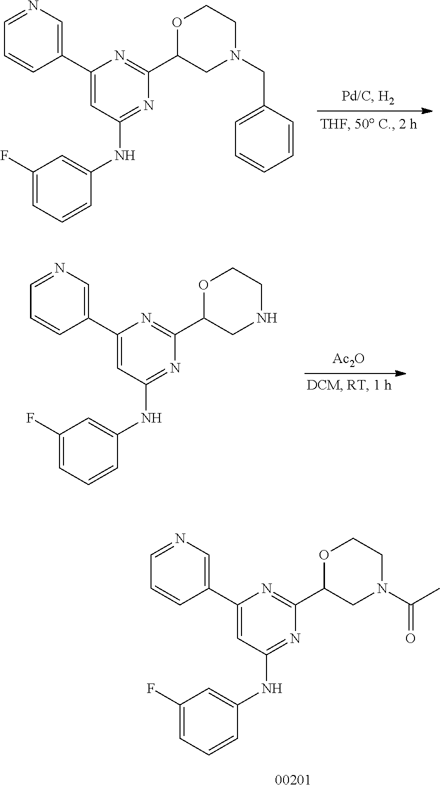

To a solution of 4-benzylmorpholine-2-carbonitrile (5 g, 24.7 mmol) in ethanol (50 mL) was added hydroxylamine solution (50% in water, 4.54 mL, 74.2 mmol) and the mixture was stirred at 75° C. for 2 hours. The mixture was concentrated and coevaporated with ethyl acetate twice to afford 4-benzyl-N-hydroxymorpholine-2-carboximidamide (5.12 g, 88%) as a light yellow oil, which was used without further purification in the next step. LCMS (Method A): t_{R} 1.67 min, 100%, MS (ESI) 236.2 (M+H)⁺. Under argon atmosphere, 4-benzyl-N-hydroxymorpholine-2-carboximidamide (5.12 g, 21.8 mmol), acetic acid (2.49 ml, 43.5 mmol) and Raney Nickel (50% in water, 2 mL) were dissolved in methanol (50 mL). Hydrogen atmosphere was introduced and the resulting mixture was heated to 50° C. for 4 hours. The mixture was flushed with nitrogen, filtered through Celite, washed with methanol and the filtrate was concentrated to afford 4-benzylmorpholine-2-carboximidamide acetate (9.2 g, 100%) as a green solid, which was used without further purification. LCMS (Method C): t_{R}1.61 min, 100%, MS (ESI) 220.1 (M+H)⁺. A solution of 4-benzylmorpholine-2-carboximidamide acetate (6.1 g, 21.84 mmol) and dimethyl malonate (2.75 mL, 24.02 mmol) were dissolved in 1M sodium methoxide in methanol (100 mL, 100 mmol) and stirred at 65° C. for 2 hours. The mixture was cooled to room temperature and concentrated to afford 2-(4-benzylmorpholin-2-yl)pyrimidine-4,6-diol (14.2 g, 100%) as a grey solid, which was used without further purification in the next step. LCMS (Method C): t_{R} 1.44 min, 100%, MS (ESI) 288.2 (M+H)⁺. A mixture of 2-(4-benzylmorpholin-2-yl)pyrimidine-4,6-diol (5 g, 17.40 mmol) in phosphorus oxychloride (30 mL, 322 mmol) was heated at 50° C. for 20 hours. The mixture was concentrated, partitioned between water and ethyl acetate and the layers were separated. The aqueous layer was extracted with ethyl acetate twice, the combined organic layers were dried with sodium sulfate and concentrated in vacuo. The residue was purified using silica flash column chromatography (30% ethyl acetate in n-heptane) to afford 4-benzyl-2-(4,6-dichloropyrimidin-2-yl)morpholine (3.1 g, 52%) as a yellow oil. ¹H-NMR (400 MHz, DMSO-d6) δ 7.99 (s, 1H), 7.36-7.23 (m, 5H), 4.62 (dd, J=9.5, 2.6 Hz, 1H), 3.96 (dt, J=11.2, 2.9 Hz, 1H), 3.73-3.66 (m, 1H), 3.65 (s, 1H), 3.59-3.51 (m, 3H), 2.95 (dt, J=11.3, 2.0 Hz, 1H), 2.72-2.64 (m, 1H), 2.28 (ddd, J=21.1, 11.1, 8.5 Hz, 2H); LCMS (Method C): t_{R} 2.14 min, 100%, MS (ESI) 324.1 (M+H)⁺. To a solution of 3-fluoroaniline (0.16 mL, 1.62 mmol) and 4-benzyl-2-(4,6-dichloropyrimidin-2-yl)morpholine (0.5 g, 1.54 mmol) in 2-propanol (8 mL) was added concentrated hydrochloric acid (0.39 mL, 4.63 mmol). The mixture was heated at 90° C. for 4 hours and was concentrated in vacuo. The residue was purified with silica column chromatography (30% ethyl acetate in n-heptane) to afford 2-(4-benzylmorpholin-2-yl)-6-chloro-N-(3-fluorophenyl)pyrimidin-4-amine (220 mg, 34%) as a white solid. ¹H-NMR (400 MHz, DMSO-d6) δ 10.10 (s, 1H), 7.73 (dd, J=11.7, 2.9 Hz, 1H), 7.42-7.20 (m, 7H), 6.88 (td, J=8.6, 2.4 Hz, 1H), 6.74 (s, 1H), 4.47 (dd, J=9.6, 2.5 Hz, 1H), 3.95 (dt, J=11.3, 2.8 Hz, 1H), 3.68 (td, J=11.0, 2.5 Hz, 1H), 3.55 (q, J=13.2 Hz, 2H), 2.97 (dt, J=11.3, 1.9 Hz, 1H), 2.76-2.64 (m, 1H), 2.32 (dd, J=11.3, 9.6 Hz, 1H), 2.22 (td, J=11.1, 3.3 Hz, 1H); LCMS (Method C): t_{R} 2.22 min, 100%, MS (ESI) 399.1 (M+H)⁺. Under argon atmosphere, a microwave vial was charged with 2-(4-benzylmorpholin-2-yl)-6-chloro-N-(3-fluorophenyl)pyrimidin-4-amine (200 mg, 0.50 mmol), pyridine-3-boronic acid (92 mg, 0.75 mmol), PdCl₂(dppf) (18.34 mg, 0.03 mmol; CAS number 72287-26-4) and sodium carbonate (106 mg, 1.00 mmol) in 1,2-dimethoxyethane (8 mL)/water (3 mL). The mixture was heated in a microwave at 90° C. for 2 hours, poured into water and extracted with ethyl acetate twice. The combined organic layers were washed with brine once, dried with sodium sulfate, concentrated in vacuo and the residue was purified with reverse phase chromatography (Method B) to afford 2-(4-benzylmorpholin-2-yl)-N-(3-fluorophenyl)-6-(pyridin-3-yl)pyrimidin-4-amine (156 mg, 65%) as a white solid. LCMS (Method C): t_{R} 2.14 min, 100%, MS (ESI) 442.1 (M+H)⁺. Under argon atmosphere, 2-(4-benzylmorpholin-2-yl)-N-(3-fluorophenyl)-6-(pyridin-3-yl)pyrimidin-4-amine (25 mg, 0.06 mmol) was dissolved in tetrahydrofuran (2 mL) and 10% palladium on carbon (6.03 mg, 5.66 μmol) was added. The mixture was heated at 50° C. for 2 hours under hydrogen atmosphere, cooled to room temperature and flushed with nitrogen. The catalyst was removed by filtration over Celite and the filtrate was concentrated to afford N-(3-fluorophenyl)-2-(morpholin-2-yl)-6-(pyridin-3-yl)pyrimidin-4-amine (20 mg, 57%) as a yellow gum, which was used without further purification in the next step. LCMS (Method C): t_{R} 1.75 min, 100%, MS (ESI) 352.1 (M+H)⁺. A solution of N-(3-fluorophenyl)-2-(morpholin-2-yl)-6-(pyridin-3-yl)pyrimidin-4-amine (20 mg, 0.03 mmol) and acetic anhydride (100 μL, 1.06 mmol) in dichloromethane (2 mL) was stirred at room temperature for 1 hour. The mixture was concentrated and the residue was purified with reverse phase chromatography (Method B) to afford 1-(2-(4-((3-fluorophenyl)amino)-6-(pyridin-3-yl)pyrimidin-2-yl)morpholino)ethan-1-one (8 mg, 60%) as a white solid. ¹H-NMR (400 MHz, DMSO-d6) mixture of rotamers δ 10.14 (s, 1H), 9.21 (dd, J=7.9, 2.3 Hz, 1H), 8.72 (d, J=4.8 Hz, 1H), 8.39 (dd, J=8.2, 6.1 Hz, 1H), 7.96-7.85 (m, 1H), 7.59 (dd, J=8.1, 4.8 Hz, 1H), 7.47-7.33 (m, 2H), 7.27 (d, J=6.4 Hz, 1H), 6.86 (t, J=8.6 Hz, 1H), 4.63-4.52 (m, 1H), 4.45 (dd, J=10.0, 2.9 Hz, 0.5H), 4.13-3.98 (m, 2H), 3.80-3.56 (m, 2.5H), 3.13 (dd, J=13.2, 10.0 Hz, 0.5H), 3.07-2.94 (m, 0.5H), 2.06 (s, 3H); LCMS (Method D): t_{R}2.85 min, 100%, MS (ESI) 394.1 (M+H)⁺.

### Example 17: Synthesis of N-(3-fluorophenyl)-2-(4-methylmorpholin-2-yl)-6-(pyridin-3-yl)pyrimidin-4-amine (00202)

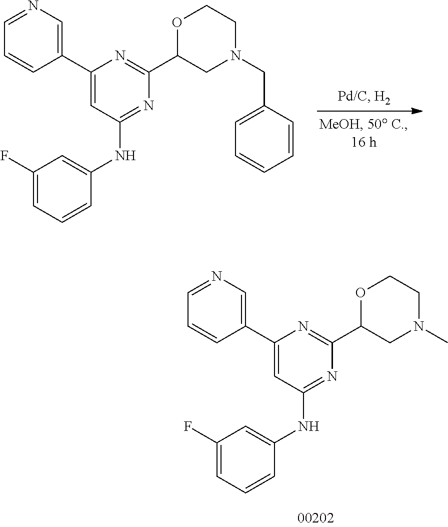 [SMILES: Fc1cccc(Nc2cc(-c3cccnc3)nc(C3CN(Cc4ccccc4)CCO3)n2)c1 ; CN1CCOC(c2nc(Nc3cccc(F)c3)cc(-c3cccnc3)n2)C1]

Under argon atmosphere, 2-(4-benzylmorpholin-2-yl)-N-(3-fluorophenyl)-6-(pyridin-3-yl)pyrimidin-4-amine (25 mg, 0.06 mmol) was dissolved in methanol (2 mL) and 10% palladium on carbon (6.03 mg, 5.66 μmol) was added. Hydrogen atmosphere was introduced and the mixture was stirred at 50° C. for 16 hours. The mixture was flushed with nitrogen, filtered over Celite and the mixture was concentrated in vacuo and the residue was purified with reverse phase chromatography (Method B) to afford N-(3-fluorophenyl)-2-(4-methylmorpholin-2-yl)-6-(pyridin-3-yl)pyrimidin-4-amine (6 mg, 27%) as a white solid. ¹H-NMR (400 MHz, DMSO-d6) δ 10.05 (s, 1H), 9.19 (d, J=2.3 Hz, 1H), 8.71 (dd, J=4.8, 1.6 Hz, 1H), 8.38 (dt, J=8.0, 2.0 Hz, 1H), 7.95 (d, J=12.1 Hz, 1H), 7.58 (dd, J=7.9, 4.8 Hz, 1H), 7.44-7.33 (m, 2H), 7.22 (s, 1H), 6.91-6.80 (m, 1H), 4.57 (dd, J=9.5, 2.5 Hz, 1H), 3.99 (dt, J=11.5, 2.8 Hz, 1H), 3.71 (td, J=11.0, 2.5 Hz, 1H), 3.02 (d, J=11.3 Hz, 1H), 2.66 (d, J=11.7 Hz, 1H), 2.43-2.35 (m, 1H), 2.25 (s, 3H), 2.19-2.09 (m, 1H); LCMS (Method D): t_{R} 2.95 min, 100%, MS (ESI) 366.1 (M+H)⁺.

### Example 18: Synthesis of N-(3-fluorophenyl)-2-(1-(methylsulfonyl)piperidin-3-yl)-6-(pyridin-3-yl)pyrimidin-4-amine (00203)

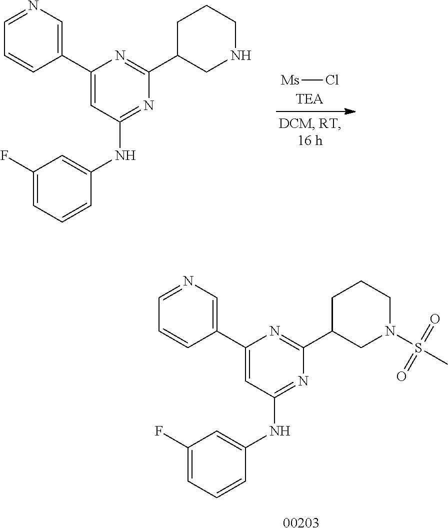 [SMILES: Fc1cccc(Nc2cc(-c3cccnc3)nc(C3CCCNC3)n2)c1 ; CS(=O)(=O)Cl ; CS(=O)(=O)N1CCCC(c2nc(Nc3cccc(F)c3)cc(-c3cccnc3)n2)C1]

To a solution of N-(3-fluorophenyl)-2-(1-(methylsulfonyl)piperidin-3-yl)-6-(pyridin-3-yl)pyrimidin-4-amine (10 mg, 0.02 mmol) and triethylamine (19.80 μL, 0.142 mmol) in dichloromethane (0.5 mL), was added mesyl chloride (6.64 μL, 0.09 mmol) and the mixture was stirred at room temperature for 16 hours. The mixture was partitioned between dichloromethane and saturated sodium bicarbonate solution. The layers were separated using a phase separator and the organic layer was concentrated in vacuo and the residue was purified with reverse phase chromatography (Method B) to afford N-(3-fluorophenyl)-2-(1-(methylsulfonyl)piperidin-3-yl)-6-(pyridin-3-yl)pyrimidin-4-amine (10 mg, 66%) as a white solid. ¹H-NMR (400 MHz, DMSO-d6) δ 10.00 (s, 1H), 9.22 (d, J=2.3 Hz, 1H), 8.76-8.67 (m, 1H), 8.43-8.35 (m, 1H), 7.92-7.83 (m, 1H), 7.62-7.54 (m, 1H), 7.44-7.33 (m, 2H), 7.18 (s, 1H), 6.90-6.80 (m, 1H), 4.02-3.92 (m, 1H), 3.63-3.50 (m, 1H), 3.13-2.97 (m, 2H), 2.90 (s, 3H), 2.85-2.74 (m, 1H), 2.29-2.19 (m, 1H), 1.96-1.86 (m, 1H), 1.84-1.62 (m, 2H); LCMS (Method D): t_{R} 3.43 min, 100%, MS (ESI) 428.1 (M+H)⁺.

### Example 19: Synthesis of N-(3-fluorophenyl)-6-(pyridin-3-yl)-2-(1-((trifluoromethyl)sulfonyl)piperidin-3-yl)pyrimidin-4-amine (00204)

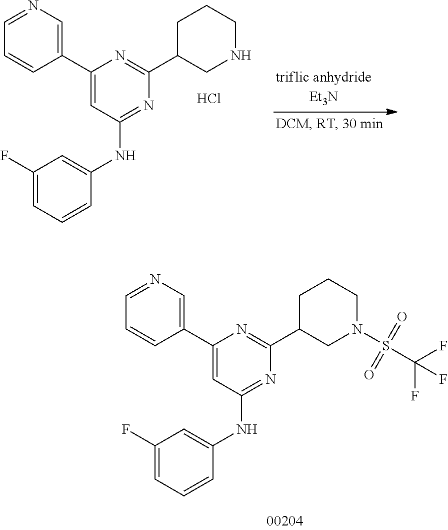 [SMILES: Fc1cccc(Nc2cc(-c3cccnc3)nc(C3CCCNC3)n2)c1 ; Cl ; O=S(=O)(N1CCCC(c2nc(Nc3cccc(F)c3)cc(-c3cccnc3)n2)C1)C(F)(F)F]

To a solution of N-(3-fluorophenyl)-2-(piperidin-3-yl)-6-(pyridin-3-yl)pyrimidin-4-amine dihydrochloride (15 mg, 0.04 mmol) and triethylamine (0.02 mL, 0.14 mmol) in dichloromethane (0.5 mL), was added triflic anhydride (6.60 μL, 0.04 mmol) and the mixture was stirred at room temperature for 30 minutes. The mixture was partitioned between dichloromethane and saturated sodium bicarbonate solution. The layers were separated using a phase separator, the organic layer was concentrated in vacuo and the residue was purified with reverse phase chromatography (Method B) to afford N-(3-fluorophenyl)-6-(pyridin-3-yl)-2-(1-((trifluoromethyl)sulfonyl)piperidin-3-yl)pyrimidin-4-amine (3 mg, 17%) as a white solid. ¹H-NMR (400 MHz, DMSO-d6) δ 10.03 (s, 1H), 9.21 (d, J=2.2 Hz, 1H), 8.76-8.65 (m, 1H), 8.44-8.32 (m, 1H), 7.89-7.80 (m, 1H), 7.63-7.54 (m, 1H), 7.41-7.32 (m, 2H), 7.19 (s, 1H), 6.91-6.80 (m, 1H), 4.30-4.14 (m, 1H), 3.90-3.77 (m, 1H), 3.70-3.51 (m, 1H), 3.37-3.22 (m, 1H), 3.12-2.99 (m, 1H), 2.37-2.29 (m, 1H), 2.02-1.81 (m, 2H), 1.80-1.63 (m, 1H); LCMS (Method D): t_{R} 3.93 min, 100%, MS (ESI) 482.1 (M+H)⁺.

### Example 20: Chiral Separation of 1-(3-(4-((3-fluorophenyl)amino)-6-(pyridin-3-yl)pyrimidin-2-yl)piperidin-1-yl)ethan-1-one (00205/00206)

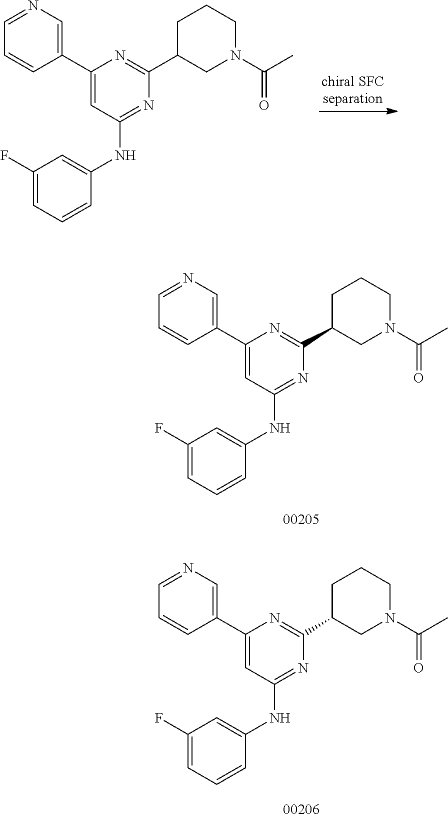 [SMILES: CC(=O)N1CCCC(c2nc(Nc3cccc(F)c3)cc(-c3cccnc3)n2)C1 ; CC(=O)N1CCC[C@H](c2nc(Nc3cccc(F)c3)cc(-c3cccnc3)n2)C1 ; CC(=O)N1CCC[C@@H](c2nc(Nc3cccc(F)c3)cc(-c3cccnc3)n2)C1]

A racemic mixture of methyl 3-(4-chloro-6-((3-fluorophenyl)amino)pyrimidin-2-yl)piperidine-1-carboxylate (32.4 mg) was separated with chiral preparative SFC (Column: SFC instrument modules: Waters Prep100q SFC System, PDA: Waters 2998, Fraction Collector: Waters 2767; Column: Phenomenex Lux Amylose-1 (250×20 mm, 5 μm), column temp: 35° C.; flow: 100 mL/min; ABPR: 170 bar; Eluent A: CO₂, Eluent B: 20 mM ammonia in methanol; isocratic 10% B, time: 30 min, detection: PDA (210-320 nm); fraction collection based on PDA) to afford S-(+)-1-(3-(4-((3-fluorophenyl)amino)-6-(pyridin-3-yl)pyrimidin-2-yl)piperidin-1-yl)ethan-1-one (12.3 mg) (00205): ¹H-NMR (400 MHz, DMSO-d6) mixture of rotamers δ 9.99 (d, J=10.4 Hz, 1H), 9.27-9.16 (m, 1H), 8.76-8.66 (m, 1H), 8.44-8.33 (m, 1H), 7.99-7.81 (m, 1H), 7.64-7.52 (m, 1H), 7.49-7.32 (m, 2H), 7.18 (d, J=4.4 Hz, 1H), 6.92-6.77 (m, 1H), 4.81-4.69 (m, 0.5H), 4.30-4.10 (m, 1H), 3.91-3.79 (m, 0.5H), 3.51 (dd, J=13.4, 10.2 Hz, 0.5H), 3.15-3.03 (m, 0.5H), 3.03-2.71 (m, 2H), 2.29-2.17 (m, 1H), 2.04 (s, 3H), 1.96-1.71 (m, 2H), 1.68-1.40 (m, 1H); ); LCMS (Method D): t_{R} 3.17 min, 100%, MS (ESI) 392.1 (M+H)⁺; specific optical rotation [α]_{D}^{24.4}: 55.7 (c=0.16, ethanol); Chiral UPLC (Method: SFC instrument modules: Waters Prep100q SFC System, PDA: Waters 2998; Column: Phenomenex Amylose-1 (100×4.6 mm, 5 μm), column temp: 35° C.; flow: 2.5 mL/min; ABPR: 170 bar; Eluent A: CO₂, Eluent B: methanol with 20 mM ammonia; t=0 min 10% B, t=8 min 40% B, t=9 min 40% B, detection: PDA (210-320 nm); fraction collection based on PDA) t_{R}4.96 min, >95% ee and R-(−)-1-(3-(4-((3-fluorophenyl)amino)-6-(pyridin-3-yl)pyrimidin-2-yl)piperidin-1-yl)ethan-1-one (12.3 mg) (00206): ¹H-NMR (400 MHz, DMSO-d6) mixture of rotamers δ 9.99 (d, J=10.4 Hz, 1H), 9.27-9.16 (m, 1H), 8.76-8.66 (m, 1H), 8.44-8.33 (m, 1H), 7.99-7.81 (m, 1H), 7.64-7.52 (m, 1H), 7.49-7.32 (m, 2H), 7.18 (d, J=4.4 Hz, 1H), 6.92-6.77 (m, 1H), 4.81-4.69 (m, 0.5H), 4.30-4.10 (m, 1H), 3.91-3.79 (m, 0.5H), 3.51 (dd, J=13.4, 10.2 Hz, 0.5H), 3.15-3.03 (m, 0.5H), 3.03-2.71 (m, 2H), 2.29-2.17 (m, 1H), 2.04 (s, 3H), 1.96-1.71 (m, 2H), 1.68-1.40 (m, 1H); ); LCMS (Method D): t_{R} 3.17 min, 100%, MS (ESI) 392.1 (M+H)⁺; specific optical rotation [α]_{D}^{24.4}: −42.5 (c=0.16, ethanol); Chiral UPLC (Method: SFC instrument modules: Waters Prep100q SFC System, PDA: Waters 2998; Column: Phenomenex Amylose-1 (4.6 mm100×4.6 mm, 5 μm), column temp: 35° C.; flow: 2.5 mL/min; ABPR: 170 bar; Eluent A: CO₂, Eluent B: methanol with 20 mM ammonia; t=0 min 10% B, t=8 min 40% B, t=9 min 40% B, detection: PDA (210-320 nm); fraction collection based on PDA) t_{R} 5.53 min, >95% ee.

The following compounds were prepared using procedures analogous to Example 20:

| Compound # | Structure and compound name | Analytical data |
|---|---|---|
| 00207 | 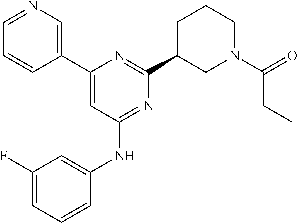 [SMILES: CCC(=O)N1CCC[C@H](c2nc(Nc3cccc(F)c3)cc(-c3cccnc3)n2)C1] S-(+)-1-(3-(4-((3- fluorophenyl)amino)-6-(pyridin-3- yl)pyrimidin-2-yl)piperidin-1- yl)propan-1-one | ¹H-NMR (400 MHz, DMSO-d6) mixture of rotamers δ 10.02 (d, J = 7.6 Hz, 1H), 9.22 (s, 1H), 8.73 (s, 1H), 8.43 (d, J = 8.0 Hz, 1H), 7.89 (dd, J = 20.4, 12.1 Hz, 1H), 7.61 (dd, J = 8.0, 4.8 Hz, 1H), 7.49 − 7.33 (m, 2H), 7.19 (d, J = 2.3 Hz, 1H), 6.85 (t, J = 8.4 Hz, 1H), 4.77 (d, J = 12.7 Hz, 0.5H), 4.23 (dd, J = 27.3, 12.7 Hz, 1H), 3.90 (d, J = 13.7 Hz, 0.5H), 3.06 (t, J = 12.7 Hz, 0.5H), 2.90 (dd, J = 21.9, 10.8 Hz, 1H), 2.84 − 2.71 (m, 1H), 2.44 − 2.31 (m, 2H), 2.23 (s, 1H), 1.99 − 1.70 (m, 2H), 1.64 − 1.36 (m, 1H), 1.07 − 0.94 (m, 3H); LCMS (Method B): t_{R} 3.09 min, 100%, MS (ESI) 406.2 (M + H)⁺; specific optical rotation [α]_{D}^{23.8}: 50.83 (c = 0.28, ethanol); Chiral LC (Method: 1_AD_DEA_IPA_80-20_30 MIN, apparatus: Agilent 1260 Quart. Pump: |
| | | G1311C, autosampler, ColCom, DAD: |
| | | Agilent G4212B, 220-320 nm, column: |
| | | Chiralcel ® AD-H 250 × 4.6 mm, 5□, |
| | | Temp: 25° C., Flow: 1 mL/min, Isocratic: |
| | | 80/20, time: 30 min, Eluent A: Heptane, |
| | | Eluent B: Isopropanol) t_{R} = 9.83 min, |
| | | >95% ee. Chiral preparative SFC |
| | | method: (Column: SFC instrument |
| | | modules: Waters Prep100q SFC |
| | | System, PDA: Waters 2998, Fraction |
| | | Collector: Waters 2767; Column: |
| | | Phenomenex Lux Amylose-1 (250 × 20 |
| | | mm, 5 μ m), column temp: 35° C.; flow: |
| | | 100 ml/min; ABPR: 170 bar; Eluent A: |
| | | CO₂, Eluent B: 20 mM ammonia in |
| | | methanol; isocratic 10% B, time: 30 min, |
| | | detection: PDA (210-320 nm); fraction |
| | | collection based on PDA). |
| 00208 | 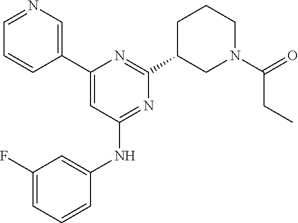 [SMILES: CCC(=O)N1CCC[C@@H](c2nc(Nc3cccc(F)c3)cc(-c3cccnc3)n2)C1] R-(−)-1-(3-(4-((3- fluorophenyl)amino)-6-(pyridin-3- yl)pyrimidin-2-yl)piperidin-1- yl)propan-1-one | ¹H-NMR (400 MHz, DMSO-d6) mixture of rotamers δ 10.00 (d, J = 9.1 Hz, 1H), 9.22 (s, 1H), 8.72 (d, J = 4.8 Hz, 1H), 8.46 − 8.35 (m, 1H), 7.90 (dd, J = 20.2, 12.1 Hz, 1H), 7.66 − 7.54 (m, 1H), 7.49 − 7.32 (m, 2H), 7.18 (d, J = 2.5 Hz, 1H), 6.85 (t, J = 8.5 Hz, 1H), 4.77 (d, J = 12.7 Hz, 0.5H), 4.23 (dd, J = 27.0, 12.9 Hz, 1H), 3.90 (d, J = 13.1 Hz, 0.5H), 3.45 (dd, J = 13.3, 10.2 Hz, 1H), 3.06 (t, J = 12.8 Hz, 0.5H), 2.98 − 2.68 (m, 2H), 2.42 − 2.29 (m, 3H), 2.23 (s, 1H), 2.01 − 1.70 (m, 2H), 1.66 − 1.36 (m, 1H), 1.06 − 0.90 (m, 3H); LCMS (Method B): t_{R} 3.09 min, 100%, MS (ESI) 406.2 (M + H)⁺; [α]_{D}^{23.9}: −56.60 (c = 0.28, ethanol); Chiral LC (Method: 1 AD DEA IPA 80-20 30 MIN, apparatus: Agilent 1260 Quart. Pump: |
| | | G1311C, autosampler, ColCom, DAD: |
| | | Agilent G4212B, 220-320 nm, column: |
| | | Chiralcel ® AD-H 250 × 4.6 mm, 5□, Temp: |
| | | 25° C., Flow: 1 mL/min, Isocratic: 80/20, |
| | | time: 30 min, Eluent A: Heptane, Eluent |
| | | B: Isopropanol) t_{R} = 8.60 min, > 95% ee. |
| | | Chiral preparative SFC method: |
| | | (Column: SFC instrument modules: |
| | | Waters Prep100q SFC System, PDA: |
| | | Waters 2998, Fraction Collector: Waters |
| | | 2767; Column: Phenomenex Lux |
| | | Amylose-1 (250 × 20 mm, 5 μ m), column |
| | | temp: 35° C.; flow: 100 ml/min; ABPR: |
| | | 170 bar; Eluent A: CO2, Eluent B: 20 |
| | | mM ammonia in methanol; isocratic 10% |
| | | B, time: 30 min, detection: PDA (210- |
| | | 320 nm); fraction collection based on |
| | | PDA). |
| 00209 | 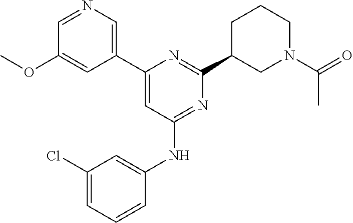 [SMILES: COc1cncc(-c2cc(Nc3cccc(Cl)c3)nc([C@H]3CCCN(C(C)=O)C3)n2)c1] S-(+)-1-(3-(4-((3- chlorophenyl)amino)-6-(5- methoxypyridin-3-yl)pyrimidin-2- yl)piperidin-1-yl)ethan-1-one | 1H-NMR (400 MHz, DMSO-d6) mixture of rotamers δ 9.97 (d, J = 12.0 Hz, 1H), 8.87 − 8.71 (m, 1H), 8.51 − 8.38 (m, 1H), 8.22 − 8.02 (m, 1H), 7.95 − 7.87 (m, 1H), 7.69 − 7.50 (m, 1H), 7.47 − 7.33 (m, 1H), 7.24 − 7.13 (m, 1H), 7.11 − 7.02 (m, 1H), 4.83 − 4.60 (m, 0.5H), 4.24 − 4.07 (m, 1H), 3.93 (s, 3H), 3.89 − 3.77 (m, 0.5H), 3.58 − 3.46 (m, 0.5H), 3.17 − 3.04 (m, 0.5H), 3.02 − 2.88 (m, 0.5H), 2.86 − 2.73 (m, 1H), 2.29 − 2.14 (m, 1H), 2.05 (d, J = 4.4 Hz, 3H), 1.97 − 1.70 (m, 2H), 1.67 − 1.40 (m, 1H); LCMS (Method B): t_{R} 3.60 min, 100%, MS (ESI) 438.2/440.2 (M + H)⁺; specific optical rotation [α]_{D}^{24.9}: 59.3 (c = 0.06, ethanol); Chiral UPLC: (Method: SFC instrument modules: Waters Prep100q SFC System, PDA: Waters 2998; |
| | | Column: Phenomenex Amylose-1 (4.6 mm |
| | | 100 × 4.6 mm, 5 μ m), column temp: |
| | | 35° C.; flow: 2.5 ml/min; ABPR: 170 bar; |
| | | Eluent A: CO₂, Eluent B: methanol with |
| | | 20 mM ammonia; t = 0 min 5% B, t = 5 min |
| | | 50% B, t = 6 min 50% B, detection: PDA |
| | | (210-320 nm); fraction collection based |
| | | on PDA) t_{R} 4.10 min, >95% ee. |
| | | Chiral preparative SFC method: |
| | | (Column: SFC instrument modules: |
| | | Waters Prep100q SFC System, PDA: |
| | | Waters 2998, Fraction Collector: Waters |
| | | 2767; Column: Phenomenex Lux |
| | | Amylose-1 (250 × 20 mm, 5 μ m), column |
| | | temp: 35° C.; flow: 100 ml/min; ABPR: |
| | | 170 bar; Eluent A: CO₂, Eluent B: 20 |
| | | mM ammonia in methanol; isocratic 10% |
| | | B, time: 30 min, detection: PDA (210- |
| | | 320 nm); fraction collection based on |
| | | PDA). |
| 00210 | 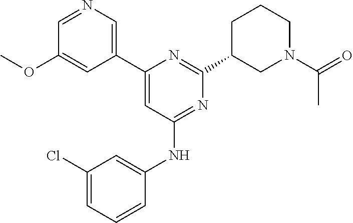 [SMILES: COc1cncc(-c2cc(Nc3cccc(Cl)c3)nc([C@@H]3CCCN(C(C)=O)C3)n2)c1] R-(−)-1-(3-(4-((3- chlorophenyl)amino)-6-(5- methoxypyriclin-3-yl)pyrimidin-2- yl)piperidin-1-yl)ethan-1-one | ¹H-NMR (400 MHz, DMSO-d6) mixture of rotamers δ 9.97 (d, J = 12.0 Hz, 1H), 8.87 − 8.71 (m, 1H), 8.51 − 8.38 (m, 1H), 8.22 − 8.02 (m, 1H), 7.95 − 7.87 (m, 1H), 7.69 − 7.50 (m, 1H), 7.47 − 7.33 (m, 1H), 7.24 − 7.13 (m, 1H), 7.11 − 7.02 (m, 1H), 4.83 − 4.60 (m, 0.5H), 4.24 − 4.07 (m, 1H), 3.93 (s, 3H), 3.89 − 3.77 (m, 0.5H), 3.58 − 3.46 (m, 0.5H), 3.17 − 3.04 (m, 0.5H), 3.02 − 2.88 (m, 0.5H), 2.86 − 2.73 (m, 1H), 2.29 − 2.14 (m, 1H), 2.05 (d, J = 4.4 Hz, 3H), 1.97 − 1.70 (m, 2H), 1.67 − 1.40 (m, 1H); LCMS (Method B): t_{R} 3.60 min, 100%, MS (ESI) 438.2/440.2 (M + H)⁺; specific optical rotation [α]_{D}^{24.8}: −62.5 (c = 0.06, ethanol); Chiral UPLC: (Method: SFC instrument modules: Waters Prep100q SFC System, PDA: Waters 2998; |
| | | Column: Phenomenex Amylose-1 (4.6 mm |
| | | 100 × 4.6 mm, 5 μ m), column temp: |
| | | 35° C.; flow: 2.5 ml/min; ABPR: 170 bar; |
| | | Eluent A: CO₂, Eluent B: methanol with |
| | | 20 mM ammonia; t = 0 min 5% B, t = 5 min |
| | | 50% B, t = 6 min 50% B, detection: PDA |
| | | (210-320 nm); fraction collection based |
| | | on PDA) t_{R} 4.57 min, >95% ee. |
| | | Chiral preparative SFC method: |
| | | (Column: SFC instrument modules: |
| | | Waters Prep100q SFC System, PDA: |
| | | Waters 2998, Fraction Collector: Waters |
| | | 2767; Column: Phenomenex Lux |
| | | Amylose-1 (250 × 20 mm, 5 μ m), column |
| | | temp: 35° C.; flow: 100 ml/min; ABPR: |
| | | 170 bar; Eluent A: CO₂, Eluent B: 20 |
| | | mM ammonia in methanol; isocratic 10% |
| | | B, time: 30 min, detection: PDA (210- |
| | | 320 nm); fraction collection based on |
| | | PDA). |

The following further compounds were prepared using procedures analogous to Example 20:

| Compound # | Structure and compound name | Analytical data |
|---|---|---|
| 00211 | 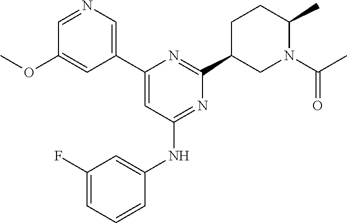 [SMILES: COc1cncc(-c2cc(Nc3cccc(F)c3)nc([C@H]3CC[C@@H](C)N(C(C)=O)C3)n2)c1] (+)1-((2R,5S)-5-(4-((3- fluorophenyl)amino)-6-(5- methoxypyridin-3-yl)pyrimidin-2-yl)- 2-methylpiperidin-1-yl)ethan-1-one | ¹H-NMR (400 MHz, CDCl₃) mixture of rotamers δ 8.74 (d, J = 16.9 Hz, 1H), 8.40 (dd, J = 7.9, 2.9 Hz, 1H), 7.90 (dt, J = 6.4, 2.1 Hz, 1H), 7.47 (ddt, J − 18.0, 10.7, 2.4 Hz, 1H), 7.34 (tt, J = 8.3, 6.3 Hz, 1H), 7.16 (dt, J = 7.9, 2.2 Hz, 1H), 7.04 − 6.95 (m, 2H), 6.87 (qd, J − 8.8, 2.5 Hz, 1H), 5.05 (p, J = 6.1, 5.4 Hz, 0.5H), 4.91 (dd, J = 13.4, 4.3 Hz, 0.5H), 4.20 (p, J = 6.5 Hz, 0.5H), 4.02 (dd, J = 13.5, 4.3 Hz, 0.5H), 3.95 (d, J = 4.7 Hz, 3H), 3.56 (dd, J = 13.7, 11.9 Hz, 0.5H), 3.09 (t, J = 12.7 Hz, 0.5H), 2.94 (tt, J = 11.8, 4.3 Hz, 1H), 2.17 (d, J = 7.8 Hz, 3H), 2.15 − 1.99 (m, 2H), 1.92 − 1.69 (m, 2H), 1.36 (d, J = 6.8 Hz, 1.5H), 1.25 (d, J = 7.0 Hz, 1.5H); LCMS (Method D): t_{R} 3.42 min, 100%, MS (ESI) 436.2 (M + H)⁺; specific optical rotation [α]_{D}^{23.8}: 36.4 (c = 0.43, methanol); Apparatus: Waters Acquity UPC²: Waters ACQ-ccBSM Binary Pump; Waters ACQ-CCM Convergence |
| | | Manager; Waters ACQ-SM Sample Manager - |
| | | Fixed Loop; Waters ACQ-CM Column |
| | | Manager - 30S; Waters ACQ-PDA Photodiode Array |
| | | Detector; Waters ACQ-ISM Make Up Pump, |
| | | Waters Acquity QDa MS Detector; Column: |
| | | Phenomenex Amylose-1 (100 × 4.6 mm 5 μm); |
| | | Column temp: 35° C.; Flow: 2.5 ml/min; Eluent |
| | | A: CO₂, Eluent B: isopropanol + 20 mM |
| | | ammonia; Gradient: t-20 min 5% B, t = 7.5 min |
| | | 30% B, t = 8 min; Detection: 210-320 nm, QDA, |
| | | ESI(scan) 100-650pos, 1Hz. t_{R} = 3.24 |
| | | min, > 95% ee. Chiral preparative SFC method: |
| | | (Column: SFC instrument modules: Waters |
| | | Prep100q SFC System, PDA: Waters 2998, |
| | | Fraction Collector: Waters 2767; Column: |
| | | Phenomenex Lux Amylose-1 (250 × 20 mm, 5 μ |
| | | m), column temp: 35° C.; flow: 100 ml/min; |
| | | ABPR: 170 bar; Eluent A: CO₂, Eluent B: 20 |
| | | mM ammonia in methanol; isocratic 10% B, |
| | | time: 30 min, detection: PDA (210-320 nm); |
| | | fraction collection based on PDA). |
| 00212 | 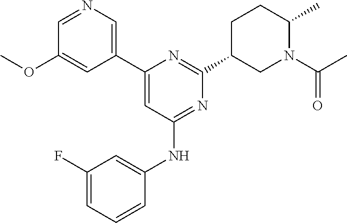 [SMILES: COc1cncc(-c2cc(Nc3cccc(F)c3)nc([C@@H]3CC[C@H](C)N(C(C)=O)C3)n2)c1] (−)-1-((2S,5R)-5-(4-((3- fluorophenyl)amino)-6-(5- methoxypyridin-3-yl)pyrimidin-2-yl)- 2-methylpiperidin-1-yl)ethan-1-one | ¹H-NMR (400 MHz, CDl₃) mixture of rotamers δ 8.74 (d, J = 16.9 Hz, 1H), 8.40 (dd, J = 7.9, 2.9 Hz, 1H), 7.90 (dt, J = 6.4, 2.1 Hz, 1H), 7.47 (ddt, J = 18.0, 10.7, 2.4 Hz, 1H), 7.34 (tt, J = 8.3, 6.3 Hz, 1H), 7.16 (dt, J = 7.9, 2.2 Hz, 1H), 7.04 − 6.95 (m, 2H), 6.87 (qd, J = 8.8, 2.5 Hz, 1H), 5.05 (p, J = 6.1, 5.4 Hz, 0.5H), 4.91 (dd, J = 13.4, 4.3 Hz, 0.5H), 4.20 (p, J = 6.5 Hz, 0.5H), 4.02 (dd, J − 13.5, 4.3 Hz, 0.5H), 3.95 (d, J = 4.7 Hz, 3H), 3.56 (dd, J = 13.7, 11.9 Hz, 0.5H), 3.09 (t, J = 12.7 Hz, 0.5H), 2.94 (tt, J = 11.8, 4.3 Hz, 1H), 2.17 (d, J = 7.8 Hz, 3H), 2.15 − 1.99 (m, 2H), 1.92 − 1.69 (m, 2H), 1.36 (d, J = 6.8 Hz, 1.5H), 1.25 (d, J = 7.0 Hz, 1.5H); LCMS (Method D): t_{R} 3.42 min, 100%, MS (ESI) 436.2 (M + H)⁺; [α]_{D}^{23.9}: −37.0 (c = 0.43, methanol); Apparatus: Waters Acquity UPC²: Waters ACQ-ccBSM Binary Pump; Waters ACQ-CCM Convergence Manager; Waters |
| | | ACQ-SM Sample Manager - Fixed Loop; |
| | | Waters ACQ-CM Column Manager - 30S; |
| | | Waters ACQ-PDA Photodiode Array Detector; |
| | | Waters ACQ-ISM Make Up Pump, Waters |
| | | Acquity QDa MS Detector; Column: |
| | | Phenomenex Amylose-1 (100 × 4.6 mm 5 μm); |
| | | Column temp: 35° C.; Flow: 2.5 ml/min; Eluent |
| | | A: CO2, Eluent B: isopropanol + 20 mM |
| | | ammonia; Gradient: t-20 min 5% B, t = 7.5 min |
| | | 30% B, t = 8 min; Detection: 210-320 nm, QDA, |
| | | ESI(scan) 100-650 pos, 1 Hz. t_{R} = 3.97 min, >95% |
| | | ee Chiral preparative SFC method: |
| | | (Column: SFC instrument modules: Waters |
| | | Prep100q SFC System, PDA: Waters 2998, |
| | | Fraction Collector: Waters 2767; Column: |
| | | Phenomenex Lux Amylose-1 (250 × 20 mm, 5 μ |
| | | m), column temp: 35° C.; flow: 100 ml/min; |
| | | ABPR: 170 bar; Eluent A: CO2, Eluent B: 20 mM |
| | | ammonia in methanol; isocratic 10% B, |
| | | time: 30 min, detection: PDA (210-320 nm); |
| | | fraction collection based on PDA). |

### Example 21: Synthesis 1-(3-(4-((3-fluorophenyl)amino)-6-phenyl-1,3,5-triazin-2-yl)piperidin-1-yl)ethan-1-one (00213)

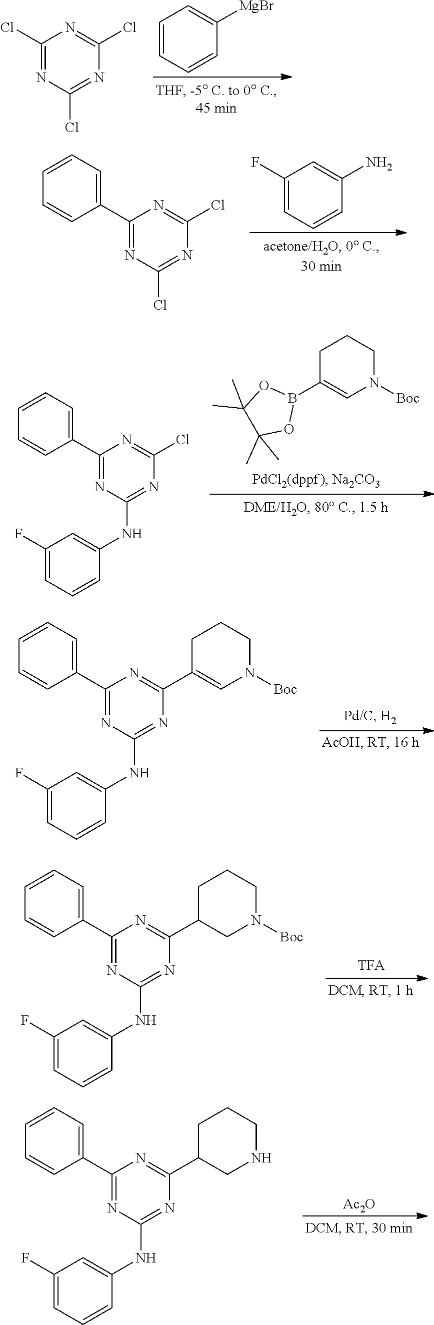

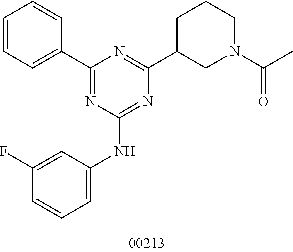 [SMILES: CC(=O)N1CCCC(c2nc(Nc3cccc(F)c3)nc(-c3ccccc3)n2)C1]

To an ice-cooled (−5° C.) solution of 2,4,6-trichloro-1,3,5-triazine (1 g, 5.43 mmol) in tetrahydrofuran (10 mL) was added 3M phenylmagnesium bromide in diethyl ether (1.99 mL, 5.97 mmol) over 15 minutes and the mixture was stirred at 0° C. for 45 minutes. Next, 1M hydrochloric acid solution (20 mL) was added and the mixture was extracted with dichloromethane once. The organic layer was washed with brine once, dried on sodium sulfate and concentrated in vacuo. The residue was purified with silica flash column chromatography (5% to 30% ethyl acetate in n-heptane) and concentrated to afford 2,4-dichloro-6-phenyl-1,3,5-triazine (917 mg, 75%) as a white solid. ¹H-NMR (400 MHz, chloroform-d) δ 8.55-8.48 (m, 2H), 7.70-7.63 (m, 1H), 7.59-7.50 (m, 2H); ¹³C-NMR (101 MHz, chloroform-d) δ 174.81, 172.05, 134.74, 132.62, 129.93, 129.06, 77.36, 77.04, 76.72. To a solution of 2,4-dichloro-6-phenyl-1,3,5-triazine (100 mg, 0.44 mmol) in a mixture of acetone (1 mL) and ice (0.5 mL), was added 3-fluoroaniline (0.04 ml, 0.44 mmol) and the mixture was vigourously stirred for 30 minutes. Saturated sodium bicarbonate solution was added, the mixture was extracted with dichloromethane and the organic layer was concentrated in vacuo. The residue was purified with silica flash column chromatography (5% to 30% ethyl acetate in n-heptane) to afford 4-chloro-N-(3-fluorophenyl)-6-phenyl-1,3,5-triazin-2-amine (95 mg, 71%) as a white solid. ¹H-NMR (400 MHz, chloroform-d) δ 8.44 (d, J=7.6 Hz, 2H), 7.77-7.57 (m, 2H), 7.56-7.43 (m, 3H), 7.41-7.30 (m, 1H), 7.30-7.23 (m, 1H), 6.97-6.81 (m, 1H); LCMS (Method C): t_{R} 2.40 min, 100%, MS (ESI) 301.0 (M+H)⁺. Under argon atmosphere, a solution of tert-butyl 5-(4,4,5,5-tetramethyl-1,3,2-dioxaborolan-2-yl)-3,4-dihydropyridine-1(2H)-carboxylate (41 mg, 0.13 mmol), 4-chloro-N-(3-fluorophenyl)-6-phenyl-1,3,5-triazin-2-amine (40 mg, 0.13 mmol), PdCl₂(dppf) (4.87 mg, 6.65 μmol; CAS number 72287-26-4) and sodium carbonate (28 mg, 0.27 mmol) in a mixture of 1,2-dimethoxyethane (3 mL) and water (1 mL) was heated at 80° C. for 1.5 hours. The mixture was diluted with water and extracted with ethyl acetate twice. The combined organic layers were washed with brine once, dried with sodium sulfate and concentrated in vacuo. The residue was purified with silica flash column chromatography (5% to 40% ethyl acetate in n-heptane to afford tert-butyl 5-(4-((3-fluorophenyl)amino)-6-phenyl-1,3,5-triazin-2-yl)-3,4-dihydropyridine-1(2H)-carboxylate (37 mg, 62%); ¹H-NMR (400 MHz, chloroform-d) δ 8.98-8.63 (m, 1H), 8.50 (d, J=7.4 Hz, 2H), 7.90-7.76 (m, 1H), 7.59-7.45 (m, 3H), 7.39-7.20 (m, 3H), 6.85-6.73 (m, 1H), 3.76-3.60 (m, 2H), 2.74-2.58 (m, 2H), 2.05-1.92 (m, 2H), 1.59 (s, 9H). Under nitrogen atmosphere, 10% palladium on carbon (11 mg, 10.34 μmol) was added to a solution of tert-butyl 5-(4-((3-fluorophenyl)amino)-6-phenyl-1,3,5-triazin-2-yl)-3,4-dihydropyridine-1(2H)-carboxylate (32 mg, 0.07 mmol) in acetic acid (3 mL) and hydrogen atmosphere was introduced. The mixture was stirred at room temperature for 16 hours, the catalyst was filtered off and the filtrate was concentrated in vacuo. The residue was purified with reverse phase chromatography (Method B) to afford tert-butyl 3-(4-((3-fluorophenyl)amino)-6-phenyl-1,3,5-triazin-2-yl)piperidine-1-carboxylate (9 mg, 27%) as a white solid. ¹H-NMR (400 MHz, chloroform-d) δ 8.53-8.44 (m, 2H), 7.85-7.74 (m, 1H), 7.62-7.47 (m, 3H), 7.39-7.27 (m, 3H), 6.88-6.78 (m, 1H), 4.62-4.25 (m, 1H), 4.25-4.00 (m, 1H), 3.33-3.08 (m, 1H), 2.98-2.77 (m, 2H), 2.31-2.21 (m, 1H), 1.88-1.75 (m, 2H), 1.71-1.60 (m, 1H), 1.48 (s, 9H); LCMS (Method C): t_{R} 2.59 min, 100%, MS (ESI) 450.2 (M+H)⁺. To a solution of tert-butyl 3-(4-((3-fluorophenyl)amino)-6-phenyl-1,3,5-triazin-2-yl)piperidine-1-carboxylate (8.9 mg, 0.02 mmol) in dichloromethane (0.3 mL) was added trifluoroacetic acid (0.3 mL, 3.89 mmol) and the mixture was stirred at room temperature for 1 hour. The mixture was concentrated in vacuo and coevaporated with dichloromethane twice. The residue was dissolved in methanol and loaded onto a SCX-2 (ion-exchange) column, washed with methanol and eluted with 1 M ammonia in methanol and concentrated in vacuo to afford N-(3-fluorophenyl)-4-phenyl-6-(piperidin-3-yl)-1,3,5-triazin-2-amine (6 mg, 87%) as a white solid. ¹H-NMR (400 MHz, chloroform-d) δ 8.57-8.39 (m, 2H), 7.90-7.76 (m, 1H), 7.66-7.46 (m, 3H), 7.40-7.27 (m, 2H), 6.85-6.76 (m, 1H), 3.49-3.40 (m, 1H), 3.28-3.08 (m, 2H), 3.03-2.82 (m, 2H), 2.27-2.14 (m, 1H), 2.01-1.90 (m, 1H), 1.88-1.75 (m, 1H), 1.73-1.58 (m, 1H); LCMS (Method C): t_{R} 2.37 min, 100%, MS (ESI) 350.1 (M+H)⁺. To a solution of N-(3-fluorophenyl)-4-phenyl-6-(piperidin-3-yl)-1,3,5-triazin-2-amine (6 mg, 0.02 mmol) in dichloromethane (0.2 mL) was added acetic anhydride (1.62 μL, 0.02 mmol) and the mixture was stirred at room temperature for 20 minutes. The mixture was concentrated and the residue was purified with reverse phase chromatography (Method B) to afford 1-(3-(4-((3-fluorophenyl)amino)-6-phenyl-1,3,5-triazin-2-yl)piperidin-1-yl)ethan-1-one (3 mg, 45%) as a white solid. ¹H-NMR (400 MHz, DMSO-d6) mixture of rotamers δ 10.49 (s, 1H), 8.48-8.36 (m, 2H), 7.89-7.79 (m, 1H), 7.70-7.55 (m, 4H), 7.41 (q, J=7.8 Hz, 1H), 6.91 (t, J=8.5 Hz, 1H), 4.79-4.69 (m, 0.5H), 4.26-4.06 (m, 1H), 3.90-3.80 (m, 0.5H), 3.62-3.51 (m, 0.5H), 3.15-3.05 (m, 0.5H), 2.99-2.69 (m, 2H), 2.28-2.15 (m, 1H), 2.05 (s, 3H), 1.96-1.71 (m, 2H), 1.66-1.40 (m, 1H); LCMS (Method D): t_{R} 3.73 min, 100%, MS (ESI) 392.1 (M+H)⁺.

### Example 22: Synthesis of N-(3-fluorophenyl)-6′-(piperidin-3-yl)-[3,4′-bipyridin]-2′-amine (00214)

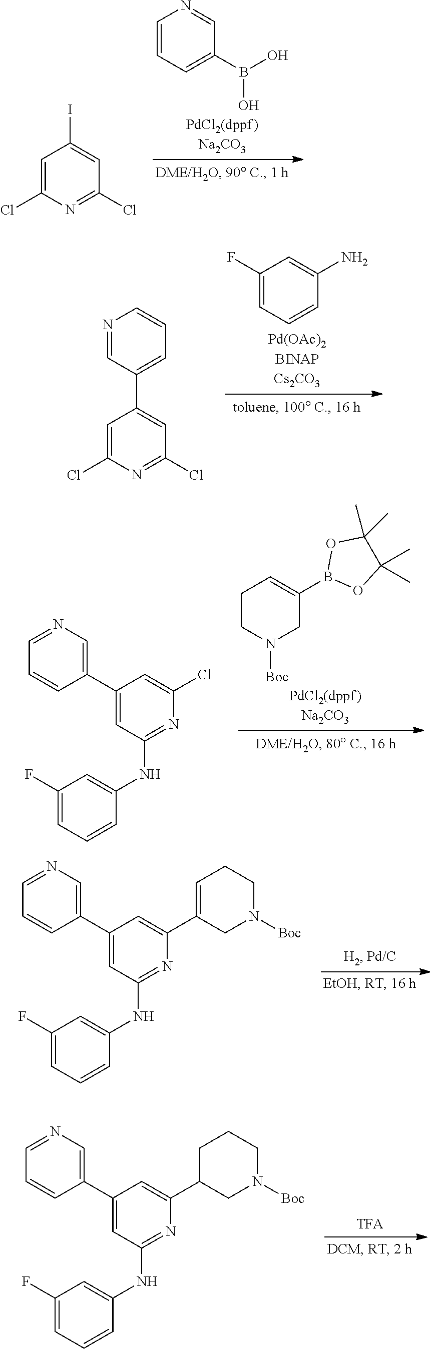

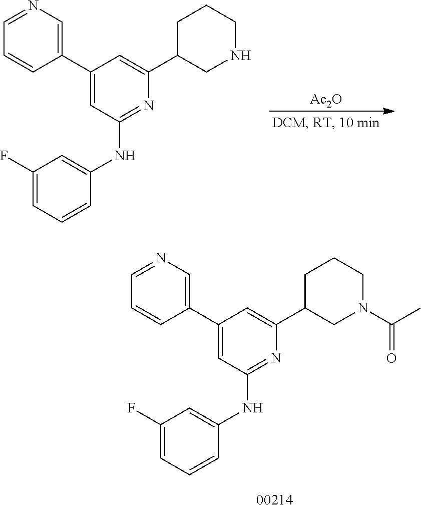 [SMILES: "Ac2O" ; Fc1cccc(Nc2cc(-c3cccnc3)cc(C3CCCNC3)n2)c1 ; CC(=O)N1CCCC(c2cc(-c3cccnc3)cc(Nc3cccc(F)c3)n2)C1]

Under argon atmosphere, pyridine-3-boronic acid (0.45 g, 3.65 mmol), 2,6-dichloro-4-iodopyridine (1 g, 3.65 mmol) and sodium carbonate (1.16 g, 10.95 mmol) were suspended in 1,2-dimethoxyethane (16 mL) and water (4 mL). The mixture was heated to 90° C., 1,1′-bis(diphenylphosphino)ferrocenepalladium(II) dichloride (0.13 g, 0.183 mmol) was added and heating was continued for 1 hour. The mixture was diluted with water and extracted with ethyl acetate twice. The combined organic layers were washed with brine, dried over sodium sulfate and concentrated in vacuo. The residue was purified with silica column chromatography (20% ethyl acetate in n-heptane) to afford 2′,6′-dichloro-3,4′-bipyridine (605 mg, 74%) as an off-white solid. ¹H-NMR (400 MHz, Chloroform-d) δ 8.87 (d, J=2.4 Hz, 1H), 8.75 (dd, J=4.7, 1.6 Hz, 1H), 7.90 (dt, J=7.9, 1.8 Hz, 1H), 7.49 (s, 2H), 7.46 (dd, J=8.5, 5.1 Hz, 1H); LCMS (Method A): t_{R} 1.70 min, 99%, MS (ESI) 224.9 (M+H)⁺. Under argon atmosphere, 2′,6′-dichloro-3,4′-bipyridine (605 mg, 2.69 mmol), 3-fluoroaniline (0.26 mL, 2.69 mmol), palladium (II) acetate (30.2 mg, 0.13 mmol), (±)-2,2′-bis(diphenylphosphino)-1,1′-binaphthyl (100 mg, 0.16 mmol) and cesium carbonate (1.31 g, 4.03 mmol) were dissolved in toluene (20 mL). The mixture was heated at 100° C. for 16 hours, filtered through celite and concentrated in vacuo. The residue was purified with silica column chromatography (25% to 60% ethyl acetate in n-heptane) to afford 6′-chloro-N-(3-fluorophenyl)-[3,4′-bipyridin]-2′-amine (182 mg, 22%) as a brown powder. ¹H-NMR (400 MHz, DMSO-d6) δ 9.77 (s, 1H), 8.94 (d, J=2.3 Hz, 1H), 8.69 (dd, J=4.9, 1.7 Hz, 1H), 8.15 (dt, J=7.7, 1.9 Hz, 1H), 7.80-7.64 (m, 1H), 7.56 (dd, J=7.8, 4.8 Hz, 1H), 7.38-7.30 (m, 2H), 7.29 (d, J=1.2 Hz, 1H), 7.09 (d, J=1.2 Hz, 1H), 6.85-6.71 (m, 1H); LCMS (Method A): t_{R} 2.03 min, 99%, MS (ESI) 300.0 (M+H)⁺. Under argon atmosphere, tert-butyl 5-(4,4,5,5-tetramethyl-1,3,2-dioxaborolan-2-yl)-3,6-dihydropyridine-1(2H)-carboxylate (67.1 mg, 0.22 mmol, synthesised according to WO 2006/47277, 2006, 97), 6′-chloro-N-(3-fluorophenyl)-[3,4′-bipyridin]-2′-amine (50 mg, 0.17 mmol) and sodium carbonate (35.4 mg, 0.33 mmol) were suspended in 1,2-dimethoxyethane (3 mL) and water (1 mL). Next, 1,1′-bis(diphenylphosphino)ferrocenepalladium(II) dichloride (6.81 mg, 8.34 μmol) was added, the mixture was heated to 80° C. for 16 hours and filtered through a nylon filter. The filtrate was dried with sodium sulfate and concentrated in vacuo. The residue was purified with silica column chromatography (40% to 80% ethyl acetate in n-heptane) to give an oil, which was recrystallized from ethyl acetate/n-heptane to afford tert-butyl 6′-((3-fluorophenyl)amino)-5,6-dihydro-[3,2′:4′,3″-terpyridine]-1(2H)-carboxylate (29 mg, 39%) as a white solid. ¹H-NMR (400 MHz, Chloroform-d) δ 8.85 (d, J=2.2 Hz, 1H), 8.68 (dd, J=4.9, 1.6 Hz, 1H), 7.88 (dt, J=7.9, 2.1 Hz, 1H), 7.49-7.33 (m, 2H), 7.32-7.23 (m, 1H), 7.17-6.98 (m, 2H), 6.97-6.78 (m, 2H), 6.78-6.61 (m, 2H), 4.53-4.39 (m, 2H), 3.69-3.50 (m, 2H), 2.46-2.31 (m, 2H), 1.51 (s, 9H); LCMS (Method C): tR 2.36 min, 97%, MS (ESI) 447.2 (M+H)⁺. Under nitrogen atmosphere, 10% palladium on activated carbon (catalytic amount) was added to a solution of tert-butyl 6′-((3-fluorophenyl)amino)-5,6-dihydro-[3,2′:4′,3″-terpyridine]-1(2H)-carboxylate (29 mg, 0.07 mmol) in ethanol (2 mL). hydrogen atmosphere was introduced and the mixture was stirred at room temperature for 16 hours. The catalyst was filtered off and the filtrate was concentrated in vacuo. The residue was purified with reverse phase chromatography (Method B) and lyophilized to afford tert-butyl 3-(6′-((3-fluorophenyl)amino)-[3,4′-bipyridin]-2′-yl)piperidine-1-carboxylate (10 mg, 36%) as a white solid. ¹H-NMR (400 MHz, Chloroform-d) δ 8.84 (d, J=2.2 Hz, 1H), 8.66 (d, J=4.7 Hz, 1H), 7.92-7.83 (m, 1H), 7.47-7.37 (m, 2H), 7.33-7.22 (m, 1H), 7.12-7.05 (m, 1H), 6.93-6.84 (m, 2H), 6.78-6.69 (m, 1H), 6.63 (s, 1H), 4.46-3.93 (m, 2H), 3.21-2.98 (m, 1H), 2.93-2.73 (m, 2H), 2.16-2.04 (m, 1H), 1.94-1.73 (m, 2H), 1.68-1.43 (m, 10H); LCMS (Method C): tR 2.36 min, 99%, MS (ESI) 449.2 (M+H)⁺. A solution of tert-butyl 3-(6′-((3-fluorophenyl)amino)-[3,4′-bipyridin]-2′-yl)piperidine-1-carboxylate (10.8 mg, 0.02 mmol) in dichloromethane (1 mL) and trifluoroacetic acid (0.2 mL, 2.60 mmol) was stirred at room temperature for 2 hours. The mixture was concentrated in vacuo and purified with SCX (ion-exchange) chromatography (washed with methanol and eluted with 3.5 M ammonia in methanol) to afford N-(3-fluorophenyl)-6′-(piperidin-3-yl)-[3,4′-bipyridin]-2′-amine (8 mg, 95%) as a colorless oil. ¹H-NMR (400 MHz, Chloroform-d) δ 8.84 (d, J=2.3 Hz, 1H), 8.70-8.62 (m, 1H), 7.91-7.82 (m, 1H), 7.46-7.35 (m, 2H), 7.32-7.21 (m, 1H), 7.15-7.07 (m, 1H), 6.92-6.78 (m, 3H), 6.76-6.68 (m, 1H), 3.38-3.27 (m, 1H), 3.16-3.06 (m, 1H), 2.99-2.90 (m, 1H), 2.90-2.81 (m, 1H), 2.78-2.68 (m, 1H), 2.17-2.07 (m, 1H), 1.90-1.77 (m, 2H), 1.70-1.54 (m, 1H); LCMS (Method C): t_{R} 2.01 min, 98%, MS (ESI) 349.2 (M+H)⁺. To a solution of N-(3-fluorophenyl)-6′-(piperidin-3-yl)-[3,4′-bipyridin]-2′-amine (8 mg, 0.02 mmol) in dichloromethane (1 mL) was added acetic anhydride (4 drops) and the mixture was stirred at room temperature for 10 minutes. Next, methanol (0.5 mL) was added and the mixture was concentrated in vacuo. The residue was purified with reverse phase chromatography (Method B) and lyophilized to afford N-(3-fluorophenyl)-6′-(piperidin-3-yl)-[3,4′-bipyridin]-2′-amine (8 mg, 89%) as a white solid. ¹H-NMR (400 MHz, DMSO-d6) a mixture of rotamers δ 9.46 (d, J=10.7 Hz, 1H), 8.97-8.88 (m, 1H), 8.70-8.63 (m, 1H), 8.16-8.08 (m, 1H), 7.98-7.86 (m, 1H), 7.60-7.51 (m, 1H), 7.38-7.25 (m, 2H), 7.12 (d, J=14.8 Hz, 1H), 6.99 (d, J=3.7 Hz, 1H), 6.74-6.64 (m, 1H), 4.66-4.56 (m, 0.5H), 4.51-4.42 (m, 0.5H), 4.15-4.05 (m, 0.5H), 3.96-3.83 (m, 0.5H), 3.33-3.21 (m, 0.5H), 3.13-3.02 (m, 0.5H), 2.93-2.80 (m, 1H), 2.80-2.69 (m, 0.5H), 2.59-2.46 (m, 0.5H), 2.18-2.01 (m, 4H), 1.93-1.73 (m, 2H), 1.65-1.37 (m, 1H); LCMS (Method D): t_{R} 391.2 min, 99%, MS (ESI) 391.2 (M+H)⁺.

### Example 23: Synthesis of 1-(3-(4-((3-fluorophenyl)amino)-[2,3′-bipyridin]-6-yl)piperidin-1-yl)ethan-1-one (00215)

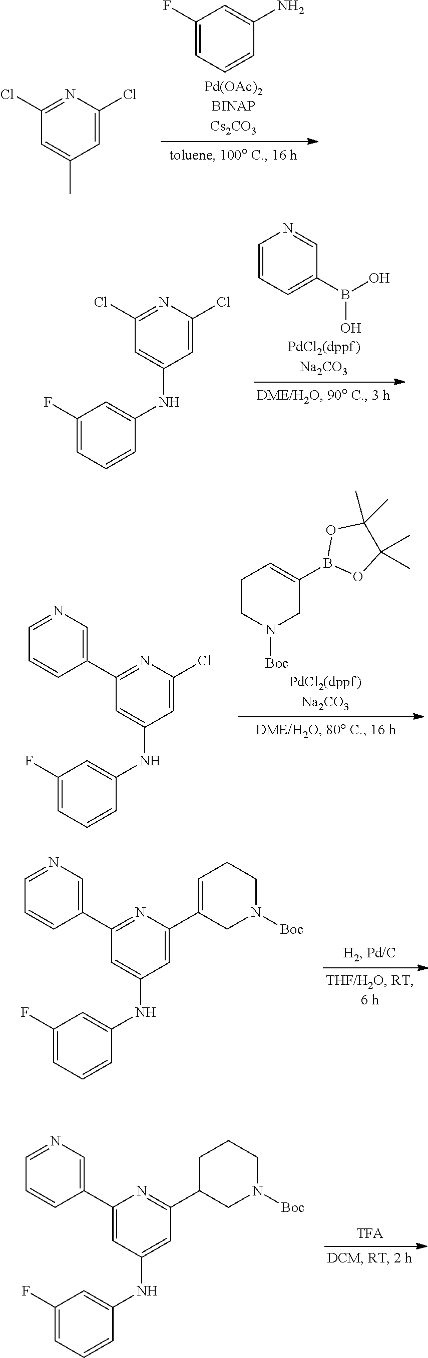

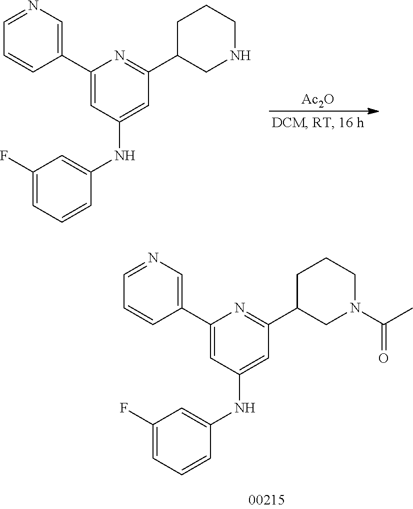 [SMILES: "Ac2O" ; Fc1cccc(Nc2cc(-c3cccnc3)nc(C3CCCNC3)c2)c1 ; CC(=O)N1CCCC(c2cc(Nc3cccc(F)c3)cc(-c3cccnc3)n2)C1]

Under argon atmosphere, 3-fluoroaniline (0.35 mL, 3.65 mmol), 2,6-dichloro-4-iodopyridine (1 g, 3.65 mmol) and cesium carbonate (1.78 g, 5.48 mmol) were dissolved in toluene (20 mL) and heated to 100° C. Palladium(II) acetate (0.04 g, 0.183 mmol) and (±)-2,2′-bis(diphenylphosphino)-1,1′-binaphthyl (0.14 g, 0.219 mmol) were added the mixture was stirred at 100° C. for 16 hours. The mixture was filtered through celite, the filter cake was rinsed with ethyl acetate and the filtrate was concentrated in vacuo. The residue was purified with silica column chromatography (5% to 50% ethyl acetate in n-heptane) to afford 2,6-dichloro-N-(3-fluorophenyl)pyridin-4-amine (800 mg, 85%) as a white solid. ¹H-NMR (400 MHz, DMSO-d6) δ 9.53 (s, 1H), 7.43 (q, J=7.7 Hz, 1H), 7.14-7.03 (m, 2H), 6.98 (td, J=8.7, 2.1 Hz, 1H), 6.89 (s, 2H); LCMS (Method A): t_{R} 2.10 min, 100%, MS (ESI) 257.0 (M+H)⁺. Under argon atmosphere, pyridine-3-boronic acid (120 mg, 0.97 mmol), sodium carbonate (103 mg, 0.97 mmol) and 2,6-dichloro-N-(3-fluorophenyl)pyridin-4-amine (250 mg, 0.97 mmol) were dissolved in 1,2-dimethoxyethane (3 mL) and water (1 mL). Next, 1,1′-bis(diphenylphosphino)ferrocenepalladium(II) dichloride (39.7 mg, 0.05 mmol) was added and the mixture was stirred at 90° C. for 3 hours. The mixture was cooled, diluted with ethyl acetate and filtered through a teflon filter. The layers were separated, the organic layer was dried with sodium sulfate, concentrated in vacuo and the residue was purified with silica column chromatography (20% to 100% ethyl acetate in n-heptane) to afford 6-chloro-N-(3-fluorophenyl)-[2,3′-bipyridin]-4-amine (100 mg, 34%) as a solid. ¹H-NMR (400 MHz, Chloroform-d) δ 9.08 (d, J=2.3 Hz, 1H), 8.65 (dd, J=4.9, 1.7 Hz, 1H), 8.29 (dt, J=8.0, 2.0 Hz, 1H), 7.42-7.34 (m, 2H), 7.16 (d, J=1.9 Hz, 1H), 7.05-6.99 (m, 1H), 6.98-6.88 (m, 2H), 6.87 (d, J=1.9 Hz, 1H), 6.32 (s, 1H); LCMS (Method C): t_{R} 2.10 min, 98%, MS (ESI) 300.1 (M+H)⁺. Under argon atmosphere, tert-butyl 5-(4,4,5,5-tetramethyl-1,3,2-dioxaborolan-2-yl)-3,6-dihydropyridine-1(2H)-carboxylate (123 mg, 0.40 mmol), 6-chloro-N-(3-fluorophenyl)-[2,3′-bipyridin]-4-amine (99 mg, 0.33 mmol) and sodium carbonate (105 mg, 0.99 mmol) were dissolved in 1,2-dimethoxyethane (3 mL) and water (1 mL). Next, 1,1′-bis(diphenylphosphino)ferrocenepalladium(II) dichloride (13.49 mg, 0.02 mmol) was added and the mixture was stirred at 80° C. for 16 hours. The mixture was diluted with ethyl acetate, filtered through and teflon filter and concentrated in vacuo. The residue was purified by reversed phase chromatography (Method B) to afford tert-butyl 4′-((3-fluorophenyl)amino)-5,6-dihydro-[3,2′:6′,3″-terpyridine]-1(2H)-carboxylate (25 mg, 17%). ¹H-NMR (400 MHz, Chloroform-d) δ 9.16 (s, 1H), 8.71-8.54 (m, 1H), 8.42-8.25 (m, 1H), 7.43-7.30 (m, 2H), 7.22 (s, 1H), 7.05-6.89 (m, 3H), 6.88-6.68 (m, 2H), 6.31 (s, 1H), 4.55-4.40 (m, 2H), 3.66-3.52 (m, 2H), 2.45-2.29 (m, 2H), 1.51 (s, 9H); LCMS (Method C): t_{R} 2.33 min, 100%, MS (ESI) 447.2 (M+H)⁺. Under nitrogen atmosphere, tert-butyl 4′-((3-fluorophenyl)amino)-5,6-dihydro-[3,2′:6′,3″-terpyridine]-1(2H)-carboxylate (25 mg, 0.06 mmol) was dissolved in tetrahydrofuran (3 mL) and water (1.5 mL) Next, 10% palladium on activated carbon (catalytic amount) was added. Hydrogen atmosphere was introduced and the mixture was stirred at room temperature for 6 hours. The mixture was diluted with methanol, filtered through a teflon filter and the filtrate was concentrated in vacuo. The residue was purified by reversed phase chromatography (Method B) to afford tert-butyl 3-(4-((3-fluorophenyl)amino)-[2,3′-bipyridin]-6-yl)piperidine-1-carboxylate (18 mg, 70%) as a colorless solid. ¹H-NMR (400 MHz, Chloroform-d) δ 9.14 (s, 1H), 8.62 (d, J=4.8 Hz, 1H), 8.35-8.27 (m, 1H), 7.43-7.30 (m, 2H), 7.17 (d, J=2.2 Hz, 1H), 7.03-6.90 (m, 2H), 6.88-6.80 (m, 1H), 6.75 (d, J=2.2 Hz, 1H), 6.18 (s, 1H), 4.46-3.94 (m, 2H), 3.18-2.97 (m, 1H), 2.91-2.69 (m, 2H), 2.14-2.01 (m, 1H), 1.96-1.72 (m, 2H), 1.70-1.56 (m, 1H), 1.47 (s, 9H); LCMS (Method C): t_{R}2.31 min, 100%, MS (ESI) 449.2 (M+H)⁺.

To a solution of tert-butyl 3-(4-((3-fluorophenyl)amino)-[2,3′-bipyridin]-6-yl)piperidine-1-carboxylate (17.6 mg, 0.04 mmol) in dichloromethane (1 mL) was added trifluoroacetic acid (0.2 mL, 2.60 mmol) and the mixture was stirred at room temperature for 1 hour. The mixture was concentrated in vacuo and the residue was purified with SCX (ion-exchange) chromatography (washed with methanol and eluted with 3.5M ammonia in methanol) to afford N-(3-fluorophenyl)-6-(piperidin-3-yl)-[2,3′-bipyridin]-4-amine (13 mg, 95%) as a colorless syrup. ¹H-NMR (400 MHz, Chloroform-d) δ 9.14 (d, J=2.3 Hz, 1H), 8.62 (dd, J=4.9, 1.7 Hz, 1H), 8.29 (dt, J=7.9, 2.0 Hz, 1H), 7.43-7.29 (m, 2H), 7.16 (d, J=1.9 Hz, 1H), 7.04-6.88 (m, 2H), 6.82 (td, J=8.3, 2.4 Hz, 1H), 6.74 (d, J=2.0 Hz, 1H), 6.23 (s, 1H), 3.36-3.25 (m, 1H), 3.16-3.04 (m, 1H), 2.98-2.88 (m, 1H), 2.88-2.77 (m, 1H), 2.77-2.65 (m, 1H), 2.16-2.05 (m, 1H), 1.90-1.76 (m, 2H), 1.66-1.53 (m, 1H); LCMS (Method C): t_{R}1.99 min, 98%, MS (ESI) 349.2 (M+H)⁺. To a solution of N-(3-fluorophenyl)-6-(piperidin-3-yl)-[2,3′-bipyridin]-4-amine (13 mg, 0.04 mmol) in dichloromethane (2 mL) was added acetic anhydride (3.52 μl, 0.04 mmol). The mixture was stirred at room temperature for 1 hour, methanol was added and the mixture was stirred for an additional 2 hours. The mixture was concentrated in vacuo and the residue was purified by reversed phase chromatography (Method B) to afford 1-(3-(4-((3-fluorophenyl)amino)-[2,3′-bipyridin]-6-yl)piperidin-1-yl)ethan-1-one (13 mg, 89%) as a colorless solid. ¹H-NMR (400 MHz, DMSO-d6) a mixture of rotamers δ 9.20-9.14 (m, 1H), 9.11 (d, J=6.1 Hz, 1H), 8.65-8.59 (m, 1H), 8.37-8.28 (m, 1H), 7.54-7.46 (m, 1H), 7.43-7.32 (m, 2H), 7.11 (d, J=8.2 Hz, 1H), 7.08-7.01 (m, 1H), 6.97-6.79 (m, 2H), 4.64-4.51 (m, 0.5H), 4.44-4.31 (m, 0.5H), 4.02-3.91 (m, 0.5H), 3.89-3.80 (m, 0.5H), 3.46-3.34 (m, 0.5H), 3.14-3.03 (m, 0.5H), 2.90-2.76 (m, 1H), 2.74-2.58 (m, 1H), 2.09-1.95 (m, 4H), 1.91-1.69 (m, 2H), 1.64-1.34 (m, 1H); LCMS (Method D): t_{R} 3.16 min, 100%, MS (ESI) 391.1 (M+H)⁺.

The following compounds were prepared using procedures analogous to Example 23:

| Compound # | Structure and compound name | Analytical data |
|---|---|---|
| 00216 | 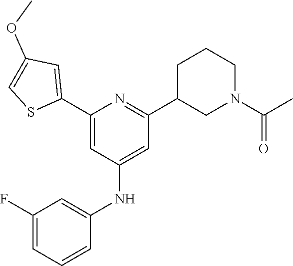 [SMILES: COc1csc(-c2cc(Nc3cccc(F)c3)cc(C3CCCN(C(C)=O)C3)n2)c1] | ¹H-NMR (400 MHz, DMSO-d6) mixture of rotamers δ 9.03 (d, J = 8.2 Hz, 1H), 7.37 (q, J = 7.9 Hz, 1H), 7.33 − 7.26 (m, 1H), 7.20 (d, J = 1.8 Hz, 1H), 7.06 (d, J = 8.2, 2.3 Hz, 1H), 7.03 − 6.96 (m, 1H), 6.88 − 6.74 (m, 2H), 6.64 (d, J = 1.7 Hz, 1H), 4.52 (d, J = 12.7 Hz, 0.5H), 4.32 (d, J = 13.0 Hz, 0.5H), 3.93 (d, J = 13.6 Hz, 0.5H), 3.83 (d, J = 13.6 Hz, 0.5H), 3.77 (s, 3H), 3.29 − 3.25 (m, 0.5H), 3.06 (t, J = 13.0 Hz, 0.5H), 2.83 − 2.55 (m, 2H), 2.02 (d, J = 3.5 Hz, 4H), 1.87 − 1.67 (m, 2H), 1.61 − 1.33 (m, 1H); LCMS (Method D): t_{R} 3.64 min, 100%, MS (ESI) 426.1 (M + H)⁺. |
| | 1-(3-(4-((3-fluorophenyl)amino)-6- | |
| | (4-methoxythiophen-2-yl)pyridin-2- | |
| | yl)piperidin-1-yl)ethan-1-one | |
| 00217 | 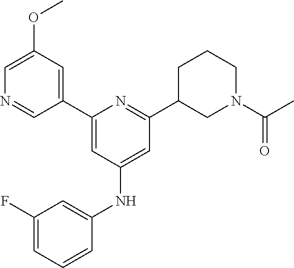 [SMILES: COc1cncc(-c2cc(Nc3cccc(F)c3)cc(C3CCCN(C(C)=O)C3)n2)c1] | ¹H-NMR (400 MHz, DMSO-d6) mixture of rotamers δ 1H NMR (400 MHz, DMSO-d6) δ 9.13 − 9.07 (m, 1H), 8.77 − 8.71 (m, 1H), 8.37 − 8.31 (m, 1H), 7.89 − 7.83 (m, 1H), 7.42 − 7.33 (m, 2H), 7.13 − 7.01 (m, 2H), 6.99 − 6.88 (m, 1H), 6.87 − 6.79 (m, 1H), 4.61 − 4.50 (m, 0.5H), 4.38 − 4.30 (m, 0.5H), 4.00 − 3.93 (m, 0.5H), 3.93 − 3.89 (m, 3H), 3.87 − 3.79 (m, 0.5H), 3.43 − 3.35 (m, 0.5H), 3.14 − 3.04 (m, 0.5H), 2.90 2.77 (m, 1H), 2.74 − 2.64 (m, 1H), 2.07 − 1.97 (m, 4H), 1.89 − 1.69 (m, 2H), 1.62 − 1.35 (m, 1H); LCMS (Method D): t_{R} 3.24 min, 100%, MS (ESI) 421.1 (M + H)⁺. |
| | 1-(3-(4-((3-fluorophenyl)amino)-5′- | |
| | methoxy-[2,3′-bipyridin]-6- | |
| | yl)piperidin-1-yl)ethan-1-one | |
| 00218 | 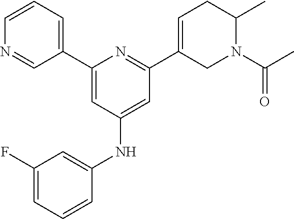 [SMILES: CC(=O)N1CC(c2cc(Nc3cccc(F)c3)cc(-c3cccnc3)n2)=CCC1C] 1-(4′-((3-fluorophenyl)amino)-6- | ¹H-NMR (400 MHz, DMSO-d6) mixture of rotamers δ 9.25 − 9.17 (m, 1H), 9.14 (s, 1H), 8.68 − 8.59 (m, 1H), 8.43 − 8.31 (m, 1H), 7.52 (dd, J = 8.0, 4.7 Hz, 1H), 7.48 − 7.34 (m, 2H), 7.22 − 7.01 (m, 3H), 6.90 − 6.80 (m, 1H), 6.80 − 6.69 (m, 1H), 5.22 − 5.05 (m, 0.6H), 4.97 − 4.88 (m, 0.4H), 4.75 − 4.60 (m, 0.4H), 4.38 − 4.21 (m, 1H), 3.86 − 3.65 (m, 0.6H), 3.38 − 3.33 (m, 1H), 2.25 − 2.15 (m, 1H), 2.15 − 2.07 (m, 3H), 1.23 − 1.16 (m, 2H), 1.10 − 1.03 (m, 1H); LCMS (Method D): t_{R} 3.27 min, 91%, MS (ESI) 403.2 (M + H)⁺. |
| | methyl-5,6-dihydro-[3,2′:6′,3″- | |
| | terpyridin]-1(2H)-yl)ethan-1-one | |
| 00219 | 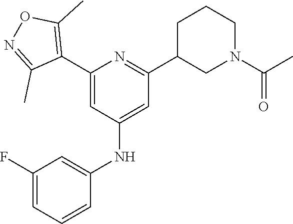 [SMILES: CC(=O)N1CCCC(c2cc(Nc3cccc(F)c3)cc(-c3c(C)noc3C)n2)C1] 1-(3-(6-(3,5-dimethylisoxazol-4-yl)- 4-((3-fluorophenyl)amino)pyridin-2- | ¹H-NMR (400 MHz, DMSO-d6) mixture of rotamers δ 9.07 (d, J = 7.4 Hz, 1H), 7.37 (q, J = 7.7 Hz, 1H), 7.10 − 6.99 (m, 2H), 6.94 (t, J = 2.2 Hz, 1H), 6.90 − 6.79 (m, 2H), 4.61 − 4.52 (m, 0.5H), 4.41 (d, J = 12.9 Hz, 0.5H), 3.95 (dd, J = 12.7, 3.4 Hz, 0.5H), 3.84 (d, J = 13.9 Hz, 0.5H), 3.27 − 3.20 (m, 0.5H), 3.09 − 2.98 (m, 0.5H), 2.84 − 2.77 (m, 0.5H), 2.77 − 2.68 (m, 0.5H), 2.65 − 2.61 (m, 0.5H), 2.58 (d, J = 2.6 Hz, 3H), 2.54 − 2.52 (m, 0.5H), 2.39 (d, J = 3.6 Hz, 3H), 2.07 − 1.94 (m, 4H), 1.85 − 1.67 (m, 2H), 1.64 − 1.32 (m, 1H); LCMS (Method B): t_{R} 2.35 min, 100%, MS (ESI) 409.2 (M + H)⁺. |
| | yl)piperidin-1-yl)ethan-1-one | |
| 00220 and 00221 | 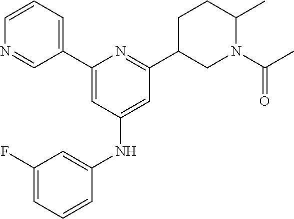 [SMILES: CC(=O)N1CC(c2cc(Nc3cccc(F)c3)cc(-c3cccnc3)n2)CCC1C] 1-(5-(4-((3-fluorophenyl)amino)- [2,3′-bipyridin]-6-yl)-2- methylpiperidin-1-y)ethan-1-one | 0220, single diastereoisomer, absolute stereochemistry unknown: ¹H-NMR (400 MHz, DMSO-d6) mixture of rotamers δ 9.23 − 9.02 (m, 1.7H), 8.65 − 8.53 (m, 1H), 8.42 − 8.19 (m, 1.3H), 7.55 − 7.43 (m, 1H), 7.43 − 7.21 (m, 2H), 7.19 − 6.89 (m, 3H), 6.89 − 6.56 (m, 1H), 4.90 − 4.41 (m, 1H), 4.21 (s, 1H), 3.14 − 3.03 (m, 1H), 2.23 − 1.69 (m, 7H), 1.51 − 1.32 (m, 1H), 1.31 − 1.05 (m, 3H); LCMS (Method D): t_{R} 3.22 min, 95%, MS (ESI) 405.2 (M + H)⁺. 00221, single diastereoisomer, absolute stereochemistry unknown: ¹H-NMR (400 MHz, DMSO-d6) mixture of rotamers δ 9.17 (dd, J = 4.7, 2.3 Hz, 1H), 9.13 (d, J = 6.0 Hz, 1H), 8.62 (d, |
| | | J = 4.6 Hz, 1H), 8.36 − 8.31 (m, 1H), |
| | | 7.54 − 7.48 (m, 1H), 7.42 − 7.35 (m, |
| | | 2H), 7.11 (d, J = 8.1 Hz, 1H), 7.09 − |
| | | 7.03 (m, 1H), 6.94 (dd, J = 22.3, 2.0 |
| | | Hz, 1H), 6.84 (td, J = 8.6, 2.5 Hz, 1H), |
| | | 4.81 (dt, J = 7.5, 3.7 Hz, 0.5H), 4.54 |
| | | (dd, J = 13.0, 4.2 Hz, 0.5H), 4.24 − |
| | | 4.14 (m, 0.5H), 3.81 (dd, J = 13.6, 4.2 |
| | | Hz, 0.5H), 3.46 (s, 0.5H), 2.90 − 2.74 |
| | | (m, 1H), 2.65 − 2.60 (m, 0.5H), 2.11 − |
| | | 1.94 (m, 5H), 1.87 − 1.76 (m, 2H), |
| | | 1.72 − 1.60 (m, 2H), 1.29 − 1.12 (m, |
| | | 3H); LCMS (Method D): t_{R} 3.22 min, |
| | | 94%, MS (ESI) 405.2 (M + H)⁺. |
| 00222 | 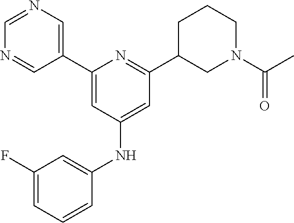 [SMILES: CC(=O)N1CCCC(c2cc(Nc3cccc(F)c3)cc(-c3cncnc3)n2)C1] | ¹H-NMR (400 MHz, DMSO-d6) mixture of rotamers δ 9.42 − 9.28 (m, 2H), 9.26 − 9.14 (m, 2H), 7.47 − 7.30 (m, 2H), 7.17 − 7.03 (m, 2H), 7.02 − 6.90 (m, 1H), 6.90 − 6.79 (m, 1H), 4.61 − 4.50 (m, 0.5H), 4.42 − 4.31 (m, 0.5H), 3.89 − 3.79 (m, 0.5H), 3.45 − 3.39 (m, 0.5H), 3.14 − 3.04 (m, 0.5H), 2.90 − 2.78 (m, 1H), 2.75 − 2.57 (m, 1H), 2.08 − 1.93 (m, 4H), 1.90 − 1.68 (m, 2H), 1.63 − 1.35 (m, 1H); LCMS (Method D): t_{R} 3.34 min, 100%, MS (ESI) 392.2 (M + H)⁺. |
| | 1-(3-(4-((3-fluorophenyl)amino)-6- | |
| | (pyrimidin-5-yl)pyridin-2- | |
| | yl)piperidin-1-yl)ethan-1-one | |

### Example 24: Synthesis of N-(3-fluorophenyl)-6-(pyridin-3-yl)-2-(1-(2,2,2-trifluoroethyl)piperidin-3-yl)pyrimidin-4-amine (00223)

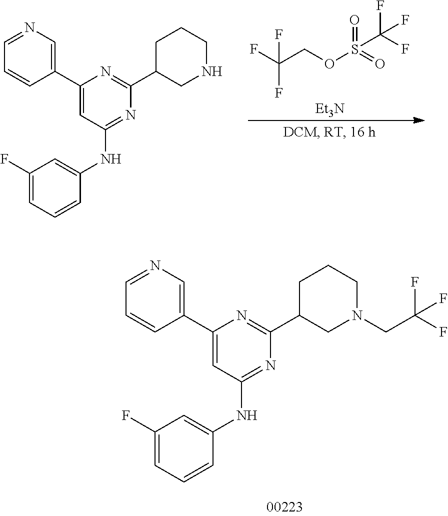 [SMILES: "Et3N" ; Fc1cccc(Nc2cc(-c3cccnc3)nc(C3CCCNC3)n2)c1 ; O=S(=O)(OCC(F)(F)F)C(F)(F)F ; Fc1cccc(Nc2cc(-c3cccnc3)nc(C3CCCN(CC(F)(F)F)C3)n2)c1]

To a solution of N-(3-fluorophenyl)-2-(piperidin-3-yl)-6-(pyridin-3-yl)pyrimidin-4-amine (10 mg, 0.03 mmol) and triethylamine (12.0 μL, 0.09 mmol) in dichloromethane (0.5 mL), was added 2,2,2-trifluoroethyl trifluoromethanesulfonate (9.96 mg, 0.04 mmol) and the mixture was stirred at room temperature for 16 hours. The mixture was concentrated in vacuo and the residue was purified by reversed phase chromatography (Method A) to afford N-(3-fluorophenyl)-6-(pyridin-3-yl)-2-(1-(2,2,2-trifluoroethyl)piperidin-3-yl)pyrimidin-4-amine (3 mg, 20%) as an off white solid. ¹H-NMR (400 MHz, DMSO-d6) δ 9.94 (s, 1H), 9.20 (s, 1H), 8.78-8.64 (m, 1H), 8.41-8.33 (m, 1H), 7.96-7.85 (m, 1H), 7.63-7.53 (m, 1H), 7.45-7.30 (m, 2H), 7.15 (s, 1H), 6.89-6.79 (m, 1H), 3.28-3.18 (m, 2H), 3.03-2.91 (m, 2H), 2.67 (t, J=10.8 Hz, 1H), 2.44-2.35 (m, 1H), 2.17-2.06 (m, 1H), 1.82-1.72 (m, 1H), 1.64 (q, J=8.2 Hz, 2H); LCMS (Method B): t_{R} 3.32 min, 100%, MS (ESI) 432.2 (M+H)⁺.

### Example 25: Synthesis of 1-(3-(6-((3-fluorophenyl)amino)-[2,3′-bipyridin]-4-yl)piperidin-1-yl)ethan-1-one (00224)

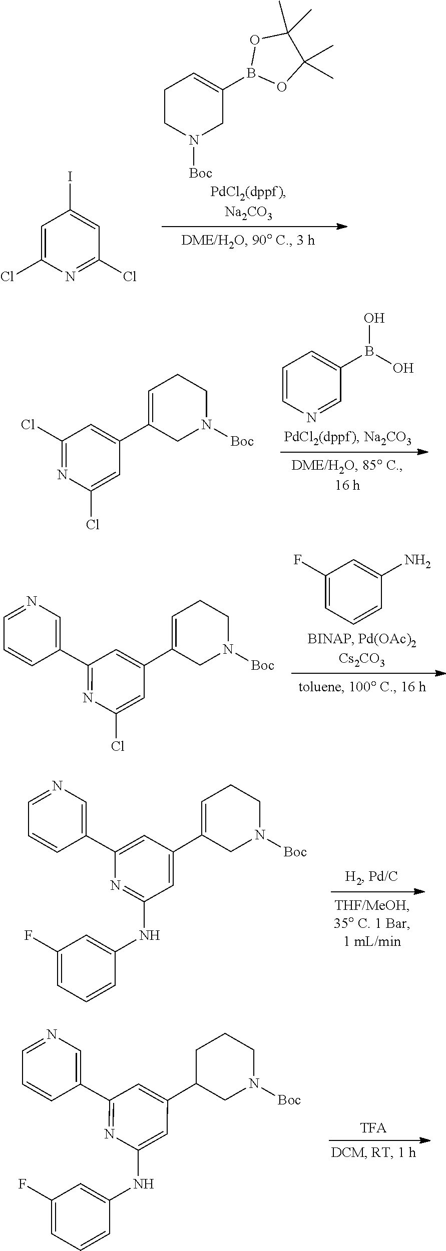

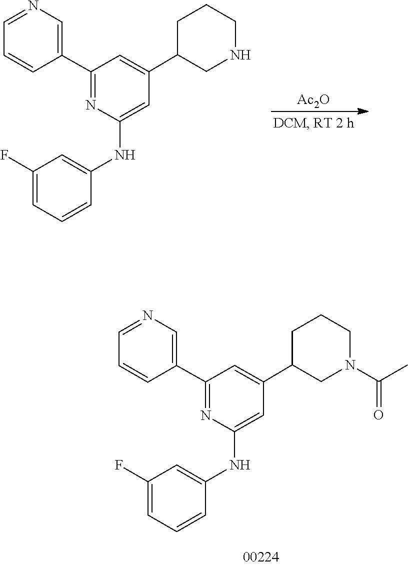 [SMILES: "Ac2O" ; Fc1cccc(Nc2cc(C3CCCNC3)cc(-c3cccnc3)n2)c1 ; CC(=O)N1CCCC(c2cc(Nc3cccc(F)c3)nc(-c3cccnc3)c2)C1]

Under argon atmosphere, 2,6-dichloro-4-iodopyridine (0.5 g, 1.83 mmol), tert-butyl 5-(4,4,5,5-tetramethyl-1,3,2-dioxaborolan-2-yl)-3,6-dihydropyridine-1(2H)-carboxylate (0.73 g, 2.37 mmol) and sodium carbonate (0.58 g, 5.48 mmol) were dissolved in 1,2-dimethoxyethane (10 mL) and water (3 mL). Next, the mixture was heated to 50° C., 1,1′-bis(diphenylphosphino)ferrocenepalladium(II) dichloride (0.08 g, 0.09 mmol) was added and the mixture was heated at 90° C. for 3 hours. The mixture was diluted with ethyl acetate, washed with brine, the layers were separated, the organic layer was dried with sodium sulfate and concentrated in vacuo. The residue was purified with silica column chromatography (5% to 30% ethyl acetate in n-heptane) to afford tert-butyl 2′,6′-dichloro-5,6-dihydro-[3,4′-bipyridine]-1(2H)-carboxylate (430 mg, 72%) as a colorless oil. ¹H-NMR (400 MHz, Chloroform-d) δ 7.22 (s, 2H), 6.52 (tt, J=4.1, 1.9 Hz, 1H), 4.20 (s, 2H), 3.55 (t, J=5.7 Hz, 2H), 2.47-2.27 (m, 2H), 1.50 (s, 9H); LCMS (Method C): t_{R} 2.32 min, 97%, MS (ESI) 329.1 (M+H)⁺. Under argon atmosphere, pyridine-3-boronic acid (160 mg, 1.30 mmol), tert-butyl 2′,6′-dichloro-5,6-dihydro-[3,4′-bipyridine]-1(2H)-carboxylate (330 mg, 1.00 mmol) and sodium carbonate (319 mg, 3.01 mmol) were dissolved in 1,2-dimethoxyethane (5 mL) and water (1 mL). Next, 1,1′-bis(diphenylphosphino)ferrocenepalladium(II) dichloride (40.9 mg, 0.05 mmol) was added and the mixture was heated in an preheated oilbath at 85° C. for 16 hours. The mixture was diluted with ethyl acetate, the layers were separated and the organic layer was dried with sodium sulfate and concentrated in vacuo. The residue was purified with silica column chromatography (20% to 80% ethyl acetate in n-heptane) to afford tert-butyl 6′-chloro-5″,6″-dihydro-[3,2′:4′,3″-terpyridine]-1″(2″H)-carboxylate (107 mg, 29%) as a yellow oil. ¹H-NMR (400 MHz, Chloroform-d) δ 9.17 (d, J=2.3 Hz, 1H), 8.67 (dd, J=4.9, 1.6 Hz, 1H), 8.33 (d, J=8.0 Hz, 1H), 7.61 (d, J=1.3 Hz, 1H), 7.41 (dd, J=8.0, 4.8 Hz, 1H), 7.34-7.23 (m, 1H), 6.60-6.51 (m, 1H), 4.30 (s, 2H), 3.59 (t, J=5.7 Hz, 2H), 2.47-2.32 (m, 2H), 1.51 (s, 9H); LCMS (Method C): t_{R} 2.17 min, 98%, MS (ESI) 372.1 (M+H)⁺. Under argon atmosphere, 3-fluoroaniline (0.03 mL, 0.32 mmol), tert-butyl 6′-chloro-5″,6″-dihydro-[3,2′:4′,3″-terpyridine]-1″(2″H)-carboxylate (107 mg, 0.29 mmol) and cesium carbonate (141 mg, 0.43 mmol) were dissolved in toluene (3 mL). The mixture was heated to 80° C., (±)-2,2′-Bis(diphenylphosphino)-1,1′-binaphthyl (10.75 mg, 0.02 mmol) and palladium (II) acetate (3.23 mg, 0.01 mmol) were added and the mixture was stirred at 100° C. for 16 hours. The mixture was diluted with ethyl acetate and filtered through a nylon filter. The filtrate was concentrated in vacuo and was purified with silica column chromatography (30% to 100% ethyl acetate in n-heptane) to afford tert-butyl 6′-((3-fluorophenyl)amino)-5″,6″-dihydro-[3,2′:4′,3″-terpyridine]-1″(2″H)-carboxylate (42 mg, 33%) as a white solid. ¹H-NMR (400 MHz, Chloroform-d) δ 9.21 (d, J=2.4 Hz, 1H), 8.65 (dd, J=4.7, 1.7 Hz, 1H), 8.30 (d, J=7.9 Hz, 1H), 7.47-7.36 (m, 2H), 7.33-7.23 (m, 2H), 7.19-7.12 (m, 1H), 6.80-6.71 (m, 2H), 6.70-6.58 (m, 1H), 6.51-6.42 (m, 1H), 4.38-4.19 (m, 2H), 3.58 (t, J=5.8 Hz, 2H), 2.44-2.29 (m, 2H), 1.51 (s, 9H); LCMS (Method C): t_{R} 2.41 min, 96%, MS (ESI) 447.1 (M+H)⁺. A solution of tert-butyl 6′-((3-fluorophenyl)amino)-5″,6″-dihydro-[3,2′:4′,3″-terpyridine]-1″(2″H)-carboxylate (42 mg, 0.09 mmol) in tetrahydrofuran (4 mL) and methanol (4 mL), was hydrogenated using a H-cube with immobilized 10% palladium on activated carbon at 35° C., 1 Bar hydrogen pressure and 1 mL/minute flow. The mixture was concentrated in vacuo to afford tert-butyl 3-(6-((3-fluorophenyl)amino)-[2,3′-bipyridin]-4-yl)piperidine-1-carboxylate (32 mg, 76%) as a white solid. ¹H NMR (400 MHz, Chloroform-d) δ 9.20 (d, J=2.3 Hz, 1H), 8.70-8.58 (m, 1H), 8.29 (dt, J=8.0, 2.0 Hz, 1H), 7.44-7.35 (m, 2H), 7.33-7.25 (m, 1H), 7.18-7.11 (m, 2H), 6.79-6.71 (m, 1H), 6.71-6.58 (m, 2H), 4.42-3.83 (m, 2H), 2.97-2.61 (m, 3H), 2.13-2.01 (m, 1H), 1.84-1.74 (m, 1H), 1.74-1.54 (m, 2H), 1.48 (s, 9H); LCMS (Method C): t_{R} 2.38 min, 95%, MS (ESI) 449.1 (M+H)⁺. To a solution of tert-butyl 3-(6-((3-fluorophenyl)amino)-[2,3′-bipyridin]-4-yl)piperidine-1-carboxylate (32 mg, 0.07 mmol) in dichloromethane (2 mL) was added trifluoroacetic acid (0.5 mL, 6.49 mmol) and the mixture was stirred at room temperature for 1 hour. The mixture was concentrated in vacuo and coevaporated with dichloromethane three times to afford an orange oil. The oil was purified with SCX (ion-exchange) chromatography (washed with methanol and eluted with 3.5M ammonia in methanol) to afford N-(3-fluorophenyl)-4-(piperidin-3-yl)-[2,3′-bipyridin]-6-amine as a yellow oil that was used directly in the next step. LCMS (Method C): t_{R} 1.95 min, 98%, MS (ESI) 349.2 (M+H)⁺. To a solution of N-(3-fluorophenyl)-4-(piperidin-3-yl)-[2,3′-bipyridin]-6-amine (0.071 mmol) in dichloromethane (3 mL) was added acetic anhydride (0.03 mL, 0.33 mmol) and the mixture was stirred at room temperature for 2 hours. The mixture was concentrated in vacuo, the residue was purified by reversed phase chromatography (Method B) to afford 1-(3-(6-((3-fluorophenyl)amino)-[2,3′-bipyridin]-4-yl)piperidin-1-yl)ethan-1-one (24 mg, 86% over two steps) as a white solid. ¹H-NMR (400 MHz, DMSO-d6) mixture of rotamers δ 9.44 (d, J=4.2 Hz, 1H), 9.33-9.22 (m, 1H), 8.69-8.57 (m, 1H), 8.46-8.34 (m, 1H), 7.95-7.86 (m, 1H), 7.60-7.36 (m, 3H), 7.36-7.26 (m, 1H), 6.82-6.66 (m, 2H), 4.55-4.39 (m, 1H), 3.98-3.80 (m, 1H), 3.28-3.18 (m, 0.5H), 3.16-3.05 (m, 0.5H), 2.81-2.70 (m, 1H), 2.65-2.54 (m, 1H), 2.10-1.94 (m, 4H), 1.87-1.70 (m, 2H), 1.65-1.35 (m, 1H); LCMS (Method D): t_{R} 3.28 min, 99%, MS (ESI) 391.1 (M+H)⁺.

### Example 26: Synthesis of 1-(4-(4-((3-fluorophenyl)amino)-6-(pyridin-3-yl)pyrimidin-2-yl)-2-azabicyclo[2.2.2]octan-2-yl)ethan-1-one (00225)

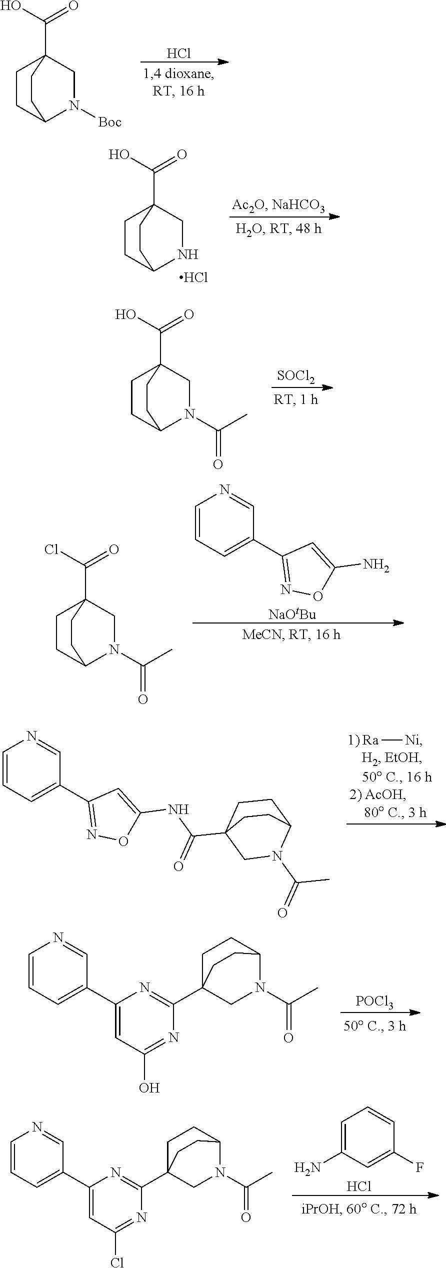

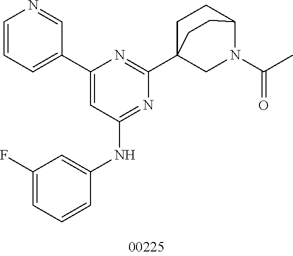 [SMILES: CC(=O)N1CC2(c3nc(Nc4cccc(F)c4)cc(-c4cccnc4)n3)CCC1CC2]

To a solution of 2-(tert-butoxycarbonyl)-2-azabicyclo[2.2.2]octane-4-carboxylic acid (250 mg, 0.98 mmol) in 1,4-dioxane (1 mL) was added 4M hydrochloric acid in dioxane (1 mL, 4.0 mmol) and the mixture was stirred at room temperature for 16 hours. The mixture was concentrated in vacuo to afford 2-azabicyclo[2.2.2]octane-4-carboxylic acid hydrochloride (190 mg, 100%) as a white solid, which was used without further purification in the next step. To a solution of 2-azabicyclo[2.2.2]octane-4-carboxylic acid hydrochloride (188 mg, 0.98 mmol) in water (4 mL), sodium bicarbonate (824 mg, 9.80 mmol) and acetic anhydride (0.93 mL, 9.80 mmol) were added. The mixture was stirred at room temperature for 48 hours, concentrated in vacuo and coevaporated with toluene twice to afford 2-acetyl-2-azabicyclo[2.2.2]octane-4-carboxylic acid (˜200 mg) as a white gum, which was used without further purification in the next step. LCMS (Method A): t_{R} 1.22 min, MS (ESI) 198.1 (M+H)⁺. A solution of 2-acetyl-2-azabicyclo[2.2.2]octane-4-carboxylic acid (180 mg, 0.91 mmol) in thionyl chloride (2 mL, 27.4 mmol) was stirred at room temperature for 1 hour. The mixture was concentrated in vacuo and redissolved in dry acetonitrile (5 mL). Under argon atmosphere, the above solution was added to a solution of 3-(pyridin-3-yl)isoxazol-5-amine (147 mg, 0.91 mmol, synthesised according to WO 2013/192046, 2013) and sodium tert-butoxide (132 mg, 1.37 mmol) in dry acetonitrile (5 mL). The mixture was stirred at room temperature for 16 hours, neutralized with saturated ammonium chloride solution (pH ˜7) and concentrated onto silica. This was purified with silica column chromatography (1% to 10% methanol in dichloromethane) to afford 2-acetyl-N-(3-(pyridin-3-yl)isoxazol-5-yl)-2-azabicyclo[2.2.2]octane-4-carboxamide (226 mg, 65%) as a yellow gum. LCMS (Method C): t_{R} 1.58 min, 90%, MS (ESI) 341.2 (M+H)⁺. Under argon atmosphere, 2-acetyl-N-(3-(pyridin-3-yl)isoxazol-5-yl)-2-azabicyclo[2.2.2]octane-4-carboxamide (226 mg, 0.664 mmol) was dissolved in ethanol (10 mL) and 50% Raney®-Nickel slurry in water (catalytic amount) was added. Next, hydrogen atmosphere was introduced via a syringe and the mixture was stirred at 50° C. for 16 hours. The mixture was filtered through Celite and concentrated in vacuo to afford 2-acetyl-N-(3-amino-3-(pyridin-3-yl)acryloyl)-2-azabicyclo[2.2.2]octane-4-carboxamide as a yellow gum. The gum was dissolved in acetic acid (5 mL) and heated at 80° C. for 3 hours. The mixture was concentrated and coevaporated with toluene twice to afford an orange oil that was purified by reversed phase chromatography (Method A) to afford 1-(4-(4-hydroxy-6-(pyridin-3-yl)pyrimidin-2-yl)-2-azabicyclo[2.2.2]octan-2-yl)ethan-1-one (55 mg) as a white solid. LCMS (Method B): t_{R} 1.48 min, MS (ESI) 325.2 (M+H)⁺. A solution of 1-(4-(4-hydroxy-6-(pyridin-3-yl)pyrimidin-2-yl)-2-azabicyclo[2.2.2]octan-2-yl)ethan-1-one (50 mg, 0.08 mmol) in phosphorus oxychloride (2 mL, 21.46 mmol) was stirred at 50° C. for 3 hours. The reaction mixture was concentrated in vacuo, diluted with water and saturated sodium bicarbonate solution and extracted with ethyl acetate twice. The combined organic layers were dried over sodium sulfate and concentrated in vacuo to afford 1-(4-(4-chloro-6-(pyridin-3-yl)pyrimidin-2-yl)-2-azabicyclo[2.2.2]octan-2-yl)ethan-1-one (32 mg, 100%) as a yellow gum. LCMS (Method C): t_{R} 1.85 min, 100%, MS (ESI) 343.1 (M+H)⁺. To a solution of 1-(4-(4-chloro-6-(pyridin-3-yl)pyrimidin-2-yl)-2-azabicyclo[2.2.2]octan-2-yl)ethan-1-one (30 mg, 0.09 mmol) and 3-fluoroaniline (0.03 mL, 0.26 mmol) in 2-propanol (3 mL) was added concentrated hydrochloric acid (0.02 mL, 0.26 mmol). The mixture was stirred at 60° C. for 72 hours and concentrated in vacuo. The residue was purified by reversed phase chromatography (Method B to afford 1-(4-(4-((3-fluorophenyl)amino)-6-(pyridin-3-yl)pyrimidin-2-yl)-2-azabicyclo[2.2.2]octan-2-yl)ethan-1-one (14 mg, 35%) as a white solid. ¹H-NMR (400 MHz, DMSO-d6) mixture of rotamers δ 10.00 (d, J=14.2 Hz, 1H), 9.22 (dd, J=5.8, 2.2 Hz, 1H), 8.71 (dd, J=4.9, 1.6 Hz, 1H), 8.44-8.35 (m, 1H), 7.95 (d, J=12.3 Hz, 0.5H), 7.90-7.83 (m, 0.5H), 7.58 (dd, J=8.0, 4.8 Hz, 1H), 7.47-7.32 (m, 2H), 7.17 (s, 1H), 6.84 (td, J=8.0, 7.4, 2.2 Hz, 1H), 4.47 (s, 0.5H), 3.91 (d, J=10.5 Hz, 1.5H), 3.70 (s, 1H), 2.23-2.08 (m, 2H), 2.02 (d, J=7.2 Hz, 3H), 1.97-1.75 (m, 6H); LCMS (Method D): t_{R} 3.40 min, 100%, MS (ESI) 418.1 (M+H)⁺.

The following compounds were prepared using procedures analogous to Example 26.

| Compound # | Structure and compound name | Analytical data |
|---|---|---|
| 00226 | 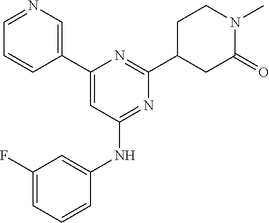 [SMILES: CN1CCC(c2nc(Nc3cccc(F)c3)cc(-c3cccnc3)n2)CC1=O] | ¹H NMR (400 MHz, DMSO-d6) δ 10.00 (s, 1H), 9.20 (d, J = 2.2 Hz, 1H), 8.71 (dd, J = 4.9, 1.7 Hz, 1H), 8.38 (dt, J = 8.1, 2.1 Hz, 1H), 7.82 (dt, J = 12.0, 2.4 Hz, 1H), 7.57 (dd, J = 8.0, 4.8 Hz, 1H), 7.47 − 7.42 (m, 1H), 7.37 (q, J = 7.8 Hz, 1H), 7.19 (s, 1H), 6.84 (td, J = 8.4, 2.6 Hz, 1H), 3.46 − 3.35 (m, 2H), 3.32 − 3.23 (m, 1H), 2.84 (s, 3H), 2.73 − 2.61 (m, 2H), 2.36 − 2.26 (m, 1H), 2.12 − 2.00 (m, 1H); LCMS (Method D): t_{R} 3.01 min, 100%, MS (ESI) 378.1 (M + H)⁺ |
| | 4-(4-((3-fluorophenyl)amino)-6- | |
| | (pyridin-3-yl)pyrimidin-2-yl)-1- | |
| | methylpiperidin-2-one | |
| 00227 | 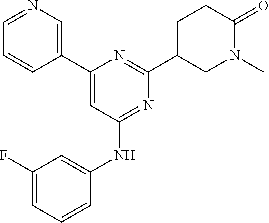 [SMILES: CN1CC(c2nc(Nc3cccc(F)c3)cc(-c3cccnc3)n2)CCC1=O] | ¹H NMR (400 MHz, DMSO-d6) δ 10.02 (s, 1H), 9.21 (d, J = 2.2 Hz, 1H), 8.71 (dd, J = 4.9, 1.6 Hz, 1H), 8.39 (dt, J = 8.1, 2.1 Hz, 1H), 7.83 (dt, J = 12.1, 2.4 Hz, 1H), 7.58 (dd, J = 7.9, 4.8 Hz, 1H), 7.47 − 7.34 (m, 2H), 7.20 (s, 1H), 6.85 (td, J = 8.3, 2.6 Hz, 1H), 3.80 − 3.65 (m, 2H), 3.41 − 3.35 (m, 1H), 2.90 (s, 3H), 2.45 − 2.34 (m, 1H), 2.34 − 2.23 (m, 2H), 2.22 − 2.11 (m, 1H); LCMS (Method D): t_{R} 3.02 min, 100%, MS (ESI) 378.1 (M + H)⁺ |
| | 5-(4-((3-fluorophenyl)amino)-6- | |
| | (pyridin-3-yl)pyrimidin-2-yl)-1- | |
| | methylpiperidin-2-one | |
| 00228 | 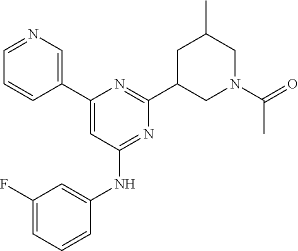 [SMILES: CC(=O)N1CC(C)CC(c2nc(Nc3cccc(F)c3)cc(-c3cccnc3)n2)C1] 1-(3-(4-((3-fluorophenyl)amino)-6- (pyridin-3-yl)pyrimidin-2-yl)-5- methylpiperidin-1-yl)ethan-1-one | ¹H NMR (400 MHz, DMSO-d6) mixture of diastereoisomers and rotamers δ 10.00 (d, J = 9.5 Hz, 1H), 9.21 (dd, J = 7.1, 2.2 Hz, 1H), 8.71 (d, J = 4.7 Hz, 1H), 8.43 − 8.35 (m, 1H), 7.90 (t, J = 13.3 Hz, 1H), 7.61 − 7.55 (m, 1H), 7.45 − 7.35 (m, 2H), 7.18 (d, J = 4.8 Hz, 1H), 6.85 (t, J = 8.4 Hz, 1H), 4.86 (d, J = 12.0 Hz, 0.5H), 4.46 (d, J = 12.7 Hz, 0.5H), 4.26 (d, J = 14.4 Hz, 0.5H), 3.84 (d, J = 13.6 Hz, 0.5H), 3.32 − 3.20 (m, 0.5H), 3.03 − 2.90 (m, 0.5H), 2.86 − 2.77 (m, 0.5H), 2.67 (t, J = 12.3 Hz, 1H), 2.34 − 2.20 (m, 1H), 2.17 − 2.04 (m, 3.5H), 1.81 − 1.68 (m, 0.5H), 1.69 − 1.54 (m, 0.5H), 1.46 (p, J = 12.5 Hz, 1H), 0.96 (t, J = 6.6 Hz, 3H); LCMS (Method D): t_{R} 3.37 min, 100%, MS (ESI) 406.2 (M + H)⁺ |
| 00229 | 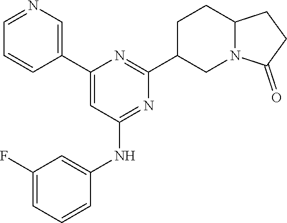 [SMILES: O=C1CCC2CCC(c3nc(Nc4cccc(F)c4)cc(-c4cccnc4)n3)CN12] (6S,8aR)-6-(4-((3- | ¹H NMR (400 MHz, DMSO-d6) δ 9.99 (s, 1H), 9.20 (d, J = 2.2 Hz, 1H), 8.71 (dd, J = 4.7, 1.6 Hz, 1H), 8.39 (dt, J = 8.1, 2.0 Hz, 1H), 7.86 (dt, J = 12.2, 2.4 Hz, 1H), 7.58 (dd, J = 8.0, 4.7 Hz, 1H), 7.46 − 7.33 (m, 2H), 7.18 (s, 1H), 6.89 − 6.80 (m, 1H), 4.34 (ddd, J = 12.4, 4.5, 1.7 Hz, 1H), 3.56 − 3.46 (m, 1H), 2.93 (t, J = 12.2 Hz, 1H), 2.77 (tt, J = 11.7, 3.9 Hz, 1H), 2.34 − 2.25 (m, 3H), 2.25 − 2.14 (m, 1H), 2.02 − 1.94 (m, 1.80 (qd, J = 13.2, 3.3 Hz, 1H), 1.62 − 1.53 (m, 1H), 1.43 − 1.31 (m, 1H); LCMS (Method D): t_{R} 3.25 min, 100%, MS (ESI) 404.2 (M + H)⁺ |
| | fluorophenyl)amino)-6-(pyridin-3- | |
| | yl)pyrimidin-2- | |
| | yl)hexahydroindolizin-3(2H)-one | |
| 00230 | 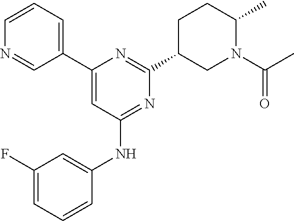 [SMILES: CC(=O)N1C[C@H](c2nc(Nc3cccc(F)c3)cc(-c3cccnc3)n2)CC[C@@H]1C] (+/−)-cis-1-(5-(4-((3- fluorophenyl)amino)-6-(pyridin-3- yl)pyrimidin-2-yl)-2-methylpiperidin- | ¹H NMR (400 MHz, DMSO-d6) mixture of rotamers δ 10.02 (d, J = 9.3 Hz, 1H), 9.21 (dd, J = 6.5, 2.2 Hz, 1H), 8.71 (d, J = 4.8 Hz, 1H), 8.40 (dd, J = 8.3, 6.3 Hz, 1H), 7.96 (t, J = 12.4 Hz, 1H), 7.58 (dd, J = 8.0, 4.8 Hz, 1H), 7.43 − 7.34 (m, 2H), 7.19 (d, J = 3.9 Hz, 1H), 6.88 − 6.81 (m, 1H), 4.89 − 4.78 (m, 0.5H), 4.72 (dd, J = 13.3, 4.2 Hz, 0.5H), 4.27 − 4.19 (m, 0.5H), 4.10 − 4.00 (m, 0.5H), 3.49 − 3.47 (m, 0.5H), 2.94 − 2.86 (m, 1H), 2.81 − 2.70 (m, 0.5H), 2.09 − 1.98 (m, 5H), 1.78 − 1.64 (m, 1.5H), 1.28 (d, J = 6.9 Hz, 1.5H), 1.15 (d, J = 7.1 Hz, 1.5H); 1H); LCMS (Method B): t_{R} 3.00 min, 100%, MS (ESI) 406.2 (M + H)⁺ |
| | 1-yl)ethan-1-one | |
| 00231 | 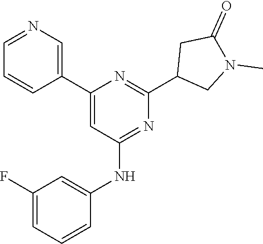 [SMILES: CN1CC(c2nc(Nc3cccc(F)c3)cc(-c3cccnc3)n2)CC1=O] | ¹H NMR (400 MHz, DMSO-d6) δ 10.03 (s, 1H), 9.20 (d, J = 2.2 Hz, 1H), 8.71 − (dd, J = 4.7, 1.6 Hz, 1H), 8.38 (dt, J = 7.9, 2.1 Hz, 1H), 7.82 (dd, J = 12.2, 2.4 Hz, 1H), 7.58 (dd, J = 8.0, 4.8 Hz, 1H), 7.44 − 7.32 (m, 2H), 7.20 (d, J = 2.2 Hz, 1H), 6.89 − 6.81 (m, 1H), 3.86 3.67 (m, 3H), 2.80 (s, 3H), 2.76 − 2.67 (m, 2H); LCMS (Method B): t_{R} 2.62 min, 100%, MS (ESI) 364.1 (M + H)⁺ |
| | 4-(4-((3-fluorophenyl)amino)-6- | |
| | (pyridin-3-yl)pyrimidin-2-yl)-1- | |
| | methylpyrrolidin-2-one | |

The following further compounds were prepared using procedures analogous to Example 26.

| Compound # | Structure and compound name | Analytical data |
|---|---|---|
| 00232 | 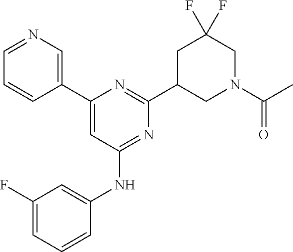 [SMILES: CC(=O)N1CC(c2nc(Nc3cccc(F)c3)cc(-c3cccnc3)n2)CC(F)(F)C1] | ¹H NMR (400 MHz, DMSO-d6) mixture of rotamers δ 10.11 (s, 1H), 9.23 (dd, J = 7.6, 2.3 Hz, 1H), 8.72 (dd, J = 4.9, 1.6 Hz, 1H), 8.50 − 8.36 (m, 1H), 7.84 (dd, J = 24.2, 12.2 Hz, 1H), 7.59 (dd, J = 7.9, 4.8 Hz, 1H), 7.50 − 7.34 (m, 2H), 7.23 (d, J = 3.8 Hz, 1H), 6.87 (t, J = 8.2 Hz, 1H), 4.90 (d, J = 12.9 Hz, 0.5H), 4.66 (s, 0.5H), 4.35 (d, J = 13.5 Hz, 0.5H), 4.23 (d, J = 14.1 Hz, 0.5H), 3.65 (dd, J = 32.4, 14.2 Hz, 0.5H), 3.56 − 3.45 (m, 0.5H), 3.29 (d, J = 13.4 Hz, 0.5H), 3.24 − 3.11 (m, 0.5H), 3.05 (d, J = 12.4 Hz, 0.5H), 2.91 (t, J = 12.1 Hz, 0.5H), 2.68 (s, 1H), 2.48 − 2.36 (m, 1H), 2.13 (d, J = 30.8 Hz, 3H); LCMS (Method D): t_{R} 3.35 min, 96%, MS (ESI) 428.1 (M + H)⁺ |
| | 1-(3,3-difluoro-5-(4-((3- | |
| | fluorophenyl)amino)-6-(pyridin-3- | |
| | yl)pyrimidin-2yl)piperidin-1- | |
| | yl)ethan-1-one | |
| 00233 | 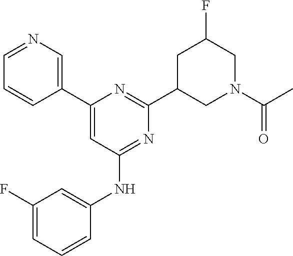 1-(3,3-difluoro-5-(4-((3- fluorophenyl)amino)-6-(pyridin-3- | ¹H NMR (400 MHz, DMSO-d6) mixture of rotamers δ 10.03 (d, J = 10.2 Hz, 1H), 9.22 (dd, J = 6.9, 2.4 Hz, 1H), 8.75 − 8.67 (m, 1H), 8.44 − 8.36 (m, 1H), 7.92 − 7.78 (m, 1H), 7.62 − 7.54 (m, 1H), 7.47 − 7.32 (m, 2H), 7.20 (d, J = 4.6 Hz, 1H), 6.91 − 6.80 (m, 1H), 5.10 (d, J = 11.8 Hz, 0.5H), 4.99 (d, J = 11.8 Hz, 0.5H), 4.82 (dd, J = 13.3, 3.2 Hz, 0.5H), 4.54 (t, J = 13.2 Hz, 0.5H), 4.25 (d, J = 14.4 Hz, 0.5H), 4.11 (t, J = 13.1 Hz, 0.5H), 3.54 − 3.44 (m, 1H), 3.44 − 3.36 (m, 1H), 3.17 − 3.02 (m, 1H), 2.99 − 2.86 (m, 1H), 2.26 − 2.16 (m, 0.5H), 2.07 (d, J = 17.7 Hz, 3.5H); LCMS (Method B): t_{R} 2.84 min, 100%, MS (ESI) 410.1 (M + H)⁺ |
| | yl)pyrimidin-2-yl)piperidin-1-yl)ethan-1-one | |
| | single diastereoisomer, relative | |
| | stereochemistry unknown | |
| 00234 | 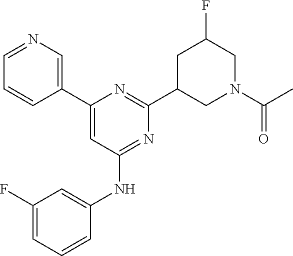 | ¹H NMR (400 MHz, DMSO-d6) mixture of rotamers δ 10.06 (d, J = 8.6 Hz, 1H), 9.22 (dd, J = 5.3, 2.3 Hz, 1H), 8.72 (dd, J = 4.8, 1.7 Hz, 1H), 8.41 (dd, J = 6.9, 3.0 Hz, 1H), 7.97 − 7.82 (m, 1H), 7.59 (dd, J = 8.1, 4.8 Hz, 1H), 7.47 − 7.32 (m, 2H), 7.21 (d, J = 2.0 Hz, 1H), 6.86 (t, J = 8.2 Hz, 1H), 4.99 − 4.55 (m, 2H), 4.27 − 4.09 (m, 1H), 3.47 − 3.37 (m, 0.5H), 3.13 (d, J = 13.7 Hz, 1H), 2.95 (d, J = 12.2 Hz, 1H), 2.77 (t, J = 12.0 Hz, 0.5H), 2.72 − 2.61 (m, 1H), 2.11 (d, J = 7.3 Hz, 3H), 2.07 − 1.94 (m, 1H); LCMS (Method B): t_{R} 2.91 min, 97%, MS (ESI) 410.1 (M + H)⁺ |
| | 1-(3,3-difluoro-5-(4-((3- | |
| | fluorophenyl)amino)-6-(pyridin-3- | |
| | yl)pyrimidin-2-yl)piperidin-1- | |
| | yl)ethan-1-one | |
| | single diastereoisomer, relative | |
| | stereochemistry unknown | |
| 00235 | 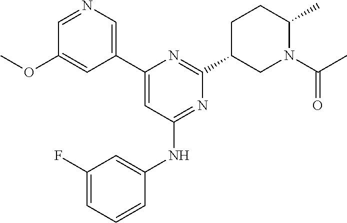 [SMILES: COc1cncc(-c2cc(Nc3cccc(F)c3)nc([C@@H]3CC[C@H](C)N(C(C)=O)C3)n2)c1] (+/−)-cis-1-(5-(4-((3- fluorophenyl)amino)-6-(5- | 1H NMR (400 MHz, DMSO-d6) mixture of rotamers δ 10.02 (d, J = 6.9 Hz, 1H), 8.83 − 8.76 (m, 1H), 8.47 − 8.39 (m, 1H), 8.03 − 7.88 (m, 2H), 7.43 − 7.34 (m, 2H), 7.19 (d, J = 4.2 Hz, 1H), 6.89 − 6.78 (m, 1H), 4.88 − 4.78 (m, 0.5H), 4.71 (dd, J = 13.3, 4.2 Hz, 0.5H), 4.21 (d, J = 6.4 Hz, 0.5H), 4.11 − 4.02 (m, 0.5H), 3.93 (s, 3H), 3.49 − 3.37 (m, 1H), 2.91 (t, J = 12.6 Hz, 1H), 2.82 − 2.69 (m, 0.5H), 2.10 − 1.95 (m, 5H), 1.91 − 1.78 (m, 0.5H), 1.71 − 1.64 (m, 1H), 1.28 (d, J = 6.8 Hz, 1.5H), 1.15 (d, J = 6.9 Hz, 1.5H); LCMS (Method D): t_{R} 3.32 min, 100%, MS (ESI) 436.2 (M + H)⁺ |
| | methoxypyridin-3-yl)pyrimidin-2-yl)- 2-methylpiperidin-1-yl)ethan-1-one | |
| 00236 | 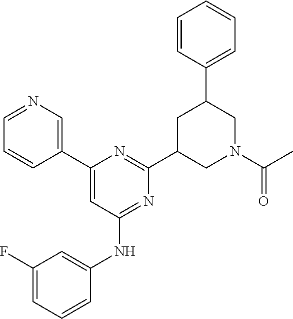 [SMILES: CC(=O)N1CC(c2ccccc2)CC(c2nc(Nc3cccc(F)c3)cc(-c3cccnc3)n2)C1] | ¹H NMR (400 MHz, DMSO-d6) mixture of rotamers δ 10.07 − 9.93 (m, 1H), 9.26 − 9.19 (m, 1H), 8.74 − 8.68 (m, 1H), 8.46 − 8.36 (m, 1H), 8.00 − 7.90 (m, 0.8H), 7.87 − 7.81 (m, 0.04H), 7.79 − 7.74 (m, 0.12H), 7.62 − 7.54 (m, 1H), 7.48 − 7.15 (m, 8H), 6.89 − 6.79 (m, 1H), 4.98 − 4.93 (m, 0.4H), 4.91 − 4.85 (m, 0.04H), 4.68 − 4.61 (m, 0.12H), 4.60 − 4.53 (m, 0.4H), 4.40 − 4.33 (m, 0.4H), 4.29 − 4.23 (m, 0.12H), 3.95 − 3.87 (m, 0.4H), 3.82 − 3.75 (m, 0.04H), 3.72 − 3.66 (m, 0.12H), 3.46 -3.38 (m, 0.6H), 3.32 − 3.26 (m, 1H), 3.26 − 2.94 (m, 2H), 2.89 − 2.61 (m, 1.5H), 2.47 − 2.39 (m, 0.5H), 2.22 − 2.04 (m, 3.5H); LCMS (Method D): t_{R} 3.64 min, 100%, MS (ESI) 468.2 (M + H)⁺ |
| | 1-(3-(4-((3-fluorophenyl)amino)-6- | |
| | (pyridin-3-yl)pyrimidin-2-yl)-5- | |
| | phenylpiperidin-1-yl)ethan-1-one | |
| 00237 | 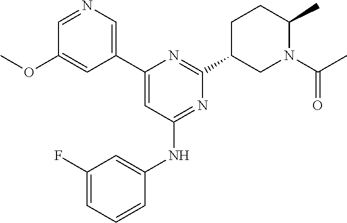 [SMILES: COc1cncc(-c2cc(Nc3cccc(F)c3)nc([C@@H]3CC[C@@H](C)N(C(C)=O)C3)n2)c1] | ¹H NMR (400 MHz, DMSO-d6) mixture of rotamers δ 10.08 − 9.83 (m, 1H), 8.79 (d, J = 1.3 Hz, 1H), 8.42 (s, 1H), 8.13 − 7.81 (m, 2H), 7.45 − 7.32 (m, 2H), 7.17 (s, 1H), 6.88 − 6.79 (m, 1H), 5.34 − 5.14 (m, 0.2H), 4.72 − 4.40 (m, 1.3H), 4.15 − 3.88 (m, 3.3H), 3.67 − 3.41 (m, 1H), 3.22 − 2.97 (m, 1.2H), 2.44 − 2.34 (m, 1H), 2.17 − 2.04 (m, 1H), 2.00 − 1.75 (m, 4H), 1.48 − 1.36 (m, 1H), 1.32 − 1.07 (m, 3H); LCMS (Method D): t_{R} 3.51 min, 99%, MS (ESI) 436.2 (M + H)⁺ |
| | (+/−)-trans-1-(5-(4-((3- fluorophenyl)amino)-6-(5- methoxypyridin-3-yl)pyrimidin-2-yl)- 2-methylpiperidin-1-yl)ethan-1-one | |
| 00238 | 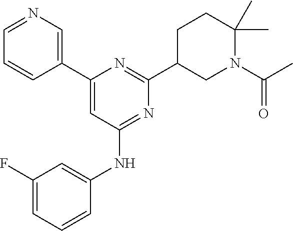 [SMILES: CC(=O)N1CC(c2nc(Nc3cccc(F)c3)cc(-c3cccnc3)n2)CCC1(C)C] | ¹H NMR (400 MHz, DMSO-d6) δ 10.01 (s, 1H), 9.21 (d, J = 2.3 Hz, 1H), 8.70 (dd, J = 4.8, 1.7 Hz, 1H), 8.38 (dt, J = 8.0, 2.0 Hz, 1H), 7.91 (dt, J = 12.2, 2.3 Hz, 1H), 7.57 (dd, J = 8.0, 4.8 Hz, 1H), 7.49 − 7.30 (m, 2H), 7.17 (s, 1H), 6.94 − 6.76 (m, 1H), 3.85 (dd, J = 13.8, 5.2 Hz, 1H), 3.64 (dd, J = 13.8, 8.6 Hz, 1H), 3.27 − 3.16 (m, 1H), 2.16 − 2.01 (m, 2H), 1.95 (s, 3H), 1.81 − 1.71 (m, 1H), 1.68 − 1.60 (m, 1H), 1.50 (s, 3H), 1.39 (s, 3H); LCMS (Method D): t_{R} 3.56 min, 96%, MS (ESI) 420.2 (M + H)⁺ |
| | 1-(5-(4-((3-fluorophenyl)amino)-6- | |
| | (pyridin-3-yl)pyrimidin-2-yl)-2,2- | |
| | dimethylpiperidin-1-yl)ethan-1-one | |
| 00239 | 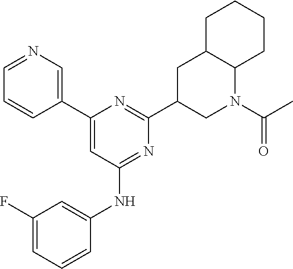 [SMILES: CC(=O)N1CC(c2nc(Nc3cccc(F)c3)cc(-c3cccnc3)n2)CC2CCCCC21] | ¹H NMR (400 MHz, DMSO-d6) mixture of diastereoisomers and rotamers δ 10.04 − 9.92 (m, 1H), 9.25 − 9.16 (m, 1H), 8.75 − 8.67 (m, 1H), 8.43 − 8.33 (m, 1H), 8.07 − 7.89 (m, 1H), 7.62 − 7.53 (m, 1H), 7.42 − 7.32 (m, 2H), 7.21 − 7.12 (m, 1H), 6.90 − 6.79 (m, 1H), 4.82 − 4.68 (m, 0.3H), 4.63 − 4.50 (m, 0.3H), 4.09 − 3.99 (m, 0.3H), 3.95 − 3.84 (m, 0.3), 3.53 − 3.39 (m, 0.6H), 3.04 − 2.94 (m, 0.3H), 2.92 − 2.75 (m, 0.6H), 2.31 − 2.03 (m, 4H), 2.02 − 1.60 (m, 6H), 1.58 − 1.05 (m, 5H); LCMS (Method B): t_{R} 3.32 min, 99%, MS (ESI) 446.2 (M + H)⁺ |
| | 1-(3-(4-((3-fluorophenyl)amino)-6- | |
| | (pyridin-3-yl)pyrimidin-2- | |
| | yl)octahydroquinolin-1(2H)-yl)ethan-1-one | |
| 00240 | 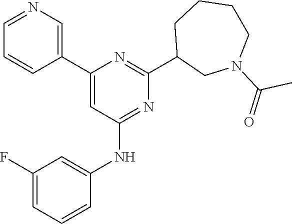 [SMILES: CC(=O)N1CCCCC(c2nc(Nc3cccc(F)c3)cc(-c3cccnc3)n2)C1] 1-(3-(4-((3-fluorophenyl)amino)-6- (pyridin-3-yl)pyrimidin-2-yl)azepan- | ¹H NMR (400 MHz, DMSO-d6) mixture of rotamers δ 9.98 (d, J = 16.8 Hz, 1H), 9.23 − 9.19 (m, 1H), 8.74 − 8.69 (m, 1H), 8.38 (ddt, J = 8.0, 4.2, 2.1 Hz, 1H), 7.96 − 7.83 (m, 1H), 7.62 − 7.53 (m, 1H), 7.45 − 7.35 (m, 2H), 7.17 (d, J = 7.5 Hz, 1H), 6.89 − 6.80 (m, 1H), 4.33 (dd, J = 13.5, 4.7 Hz, 0.5H), 4.07 (dd, J = 14.7, 4.9 Hz, 0.5H), 3.91 − 3.81 (m, 0.5H), 3.77 − 3.67 (m, 0.5H), 3.62 (dd, J = 14.8, 10.5 Hz, 0.5H), 3.44 − 3.36 (m, 0.5H), 3.21 − 3.11 (m, 1.5H), 2.14 − 1.99 (m, 4H), 1.95 − 1.78 (m, 3H), 1.77 − 1.38 (m, 2.5H); LCMS (Method D): t_{R} 3.20) min, 100%, MS (ESI) 406.2 (M + H)⁺ |
| | 1-yl)ethan-1-one | |
| 00241 | 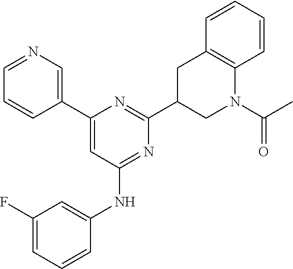 [SMILES: CC(=O)N1CC(c2nc(Nc3cccc(F)c3)cc(-c3cccnc3)n2)Cc2ccccc21] | ¹H NMR (400 MHz, DMSO-d6) δ 9.98 (s, 1H), 9.19 (d, J = 2.3 Hz, 1H), 8.82 − 8.62 (m, 1H), 8.37 (dt, J = 8.0, 2.0 Hz, 1H), 7.82 − 7.70 (m, 1H), 7.58 − 7.37 (m, 3H), 7.33 − 7.26 (m, 2H), 7.22 − 7.09 (m, 3H), 6.85 − 6.77 (m, 1H), 4.33 − 3.99 (m, 2H), 3.56 − 3.46 (m, 1H), 3.30 − 3.25 (m, 2H), 2.11 (br s, 3H); LCMS (Method D): t_{R} 3.49 min, 100%, MS (ESI) 440.1 (M + H)⁺ |
| | 1-(3-(4-((3-fluorophenyl)amino)-6- | |
| | (pyridin-3-yl)pyrimidin-2-yl)-3,4- | |
| | dihydroquinolin-1(2 H)-yl)ethan-1-one | |
| 00242 | 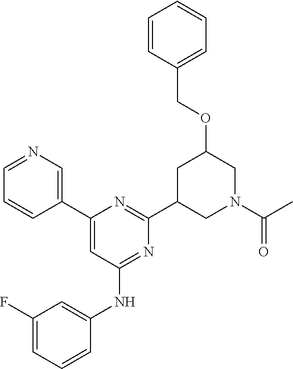 [SMILES: CC(=O)N1CC(OCc2ccccc2)CC(c2nc(Nc3cccc(F)c3)cc(-c3cccnc3)n2)C1] | ¹H NMR (400 MHz, DMSO-d6) mixture of diastereoisomers and rotamers δ 10.05 − 9.92 (m, 1H), 9.27 − 9.13 (m, 1H), 8.76 − 8.64 (m, 1H), 8.47 − 8.32 (m, 1H), 7.98 − 7.83 (m, 1H), 7.63 − 7.51 (m, 1H), 7.45 − 7.22 (m, 7H), 7.18 (dd, J = 4.4, 2.5 Hz, 1H), 6.85 (t, J = 8.2 Hz, 1H), 4.87 − 4.44 (m, 3H), 4.39 − 4.22 (m, 0.4H), 4.17 − 3.93 (m, 1H), 3.90 − 3.76 (m, 1H), 3.71 − 3.58 (m, 0.4H), 3.19 − 3.01 (m, 1H), 3.02 − 2.84 (m, 1H), 2.80 − 2.60 (m, 0.4H), 2.37 − 2.14 (m, 0.5H), 2.11 − 1.98 (m, 3H), 1.80 − 1.71 (m, 0.3H); LCMS (Method D): t_{R} 3.59 min, 99%, MS (ESI) 498.2 (M + H)⁺ |
| | 1-(3-(benzyloxy)-5-(4-((3- | |
| | fluorophenyl)amino)-6-(pyridin-3- | |
| | yl)pyrimidin-2-yl)piperidin-1- | |
| | yl)ethan-1-one | |
| 00243 | 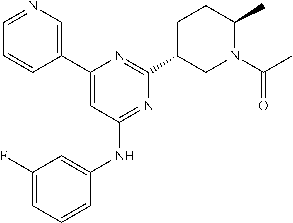 | ¹H NMR (400 MHz, DMSO-d6) mixture of rotamers δ 9.96 (s, 1H), 9.22 − 9.19 (m, 1H), 8.70 (dd, J = 4.8, 1.7 Hz, 1H), 8.38 (d, J = 7.9 Hz, 1H), 7.87 (d, J = 12.1 Hz, 1H), 7.57 (dd, J = 8.1, 4.8 Hz, 1H), 7.44 − 7.32 (m, 2H), 7.16 (s, 1H), 6.88 − 6.79 (m, 1H), 5.29 − 5.05 (m, 0.2H), 4.77 − 4.34 (m, 1.6H), 4.23 − 3.97 (m, 0.4H), 3.68 − 3.49 (m, 0.8H), 3.17 (s, 1H), 2.45 − 2.35 (m, 1H), 2.16 − 2.03 (m, 1H), 1.96 − 1.79 (m, 4H), 1.47 − 1.37 (m, 1H), 1.26 − 1.07 (m, 3H); LCMS (Method D): t_{R} 3.37 min, 99%, MS (ESI) 406.2 (M + H)⁺ |
| | (+/−)-trans-1-(5-(4-((3- | |
| | fluorophenyl)amino)-6-(pyridin-3- | |
| | yl)pyrimidin-2-yl)-2-methylpiperidin- | |
| | 1-yl)ethan-1-one | |
| 00244 | 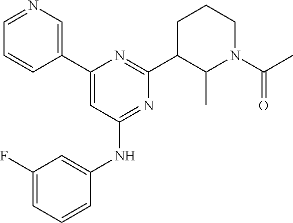 [SMILES: CC(=O)N1CCCC(c2nc(Nc3cccc(F)c3)cc(-c3cccnc3)n2)C1C] 1-(3-(4-((3-fluorophenyl)amino)-6- (pyridin-3-yl)pyrimidin-2-yl)-2- methylpiperidin-1-yl)ethan-1-one | ¹H NMR (400 MHz, DMSO-d6) mixture of diastereoisomers and rotamers δ 10.19 − 9.88 (m, 1H), 9.33 − 9.12 (m, 1H), 8.71 (dt, J = 4.9, 2.4 Hz, 1H), 8.48 − 8.32 (m, 1H), 8.06 − 7.99 (m, 0.3H), 7.97 − 7.80 (m, 0.7H), 7.62 − 7.52 (m, 1H), 7.50 − 7.31 (m, 2H), 7.26 − 7.12 (m, 1H), 6.92 − 6.77 (m, 1H), 5.49 (s, 0.1H), 5.42 -5.31 (m, 0.3H), 5.08 (s, 0.2H), 4.80 − 468(m, 0.2H), 4.38 (dd, J = 13.6, 4.4 Hz, 0.3H), 4.25 (d, J = 13.2 Hz, 0.2H), 3.78 − 3.66 (m, .5H), 3.26 − 3.11 (m, 1H), 3.08 − 2.91 (m, 0.8H), 2.78 − 2.63 (m, 0.5H), 2.47 − 2.37 (m, 0.2H), 2.32 − 1.99 (m, 34H), 1.92 (s, 1.1H), 1.88 − 1.74 (m, 0.7H), 1.64 − 1.48 (m, 0.9H), 1.49 − 1.33 (m, 1H), 1.24 (d, J = 11.2 |
| | | Hz, 0.4H), 0.97 (d, J = 6.8 Hz, 0.8H), |
| | | 0.82 (d, J = 7.0 Hz, 0.8H); LCMS |
| | | (Method D): t_{R} 3.32 min, 95%, MS |
| | | (ESI) 406.2 (M + H)⁺ |
| 00245 | 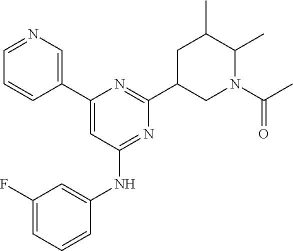 [SMILES: CC(=O)N1CC(c2nc(Nc3cccc(F)c3)cc(-c3cccnc3)n2)CC(C)C1C] | ¹H NMR (400 MHz, DMSO-d6) complex mixture of diastereoisomers and rotamers δ 10.12 − 9.93 (m, 1H), 9.20 (s, 1H), 8.71 (s, 1H), 8.48 − 8.30 (m, 1H), 8.03 − 7.83 (m, 1H), 7.57 (t, J = 6.0 Hz, 1H), 7.47 − 7.31 (m, 2H), 7.26 (s, 0.4H), 7.23 − 7.09 (m, 1.3H), 7.01 (s, 0.3H), 6.92 − 6.78 (m, 1H), 4.70 (m, 0.4H), 4.55 − 4.45 (m, 0.25H), 4.14 (s, 0.2H), 4.08 − 3.96 (m, 0.4H), 3.86 (m, 0.35H), 3.52 − 3.39 (m, 0.6H), 3.26 − 3.11 (m, 0.5H), 3.05 − 2.76 (m, 0.5H), 2.28 − 2.12 (m, 0.5H), 2.10 − 1.60 (m, 5.5H), 1.37 − 0.84 (m, 6H); LCMS (Method D): t_{R} 3.44 min, 99%, MS (ESI) 420.2 (M + H)⁺ |
| | 1-(5-(4-((3-fluorophenyl)amino)-6- | |
| | (pyridin-3-yl)pyrimidin-2-yl)-2,3- | |
| | dimethylpiperidin-1-yl)ethan-1-one | |
| 00246 | 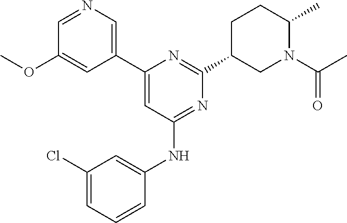 [SMILES: COc1cncc(-c2cc(Nc3cccc(Cl)c3)nc([C@@H]3CC[C@H](C)N(C(C)=O)C3)n2)c1] (+/−)-cis-1-(5-(4-((3- chlorophenyl)amino)-6-(5- methoxypyridin-3-yl)pyrimidin-2-yl)- 2-methylpiperidin-1-yl)ethan-1-one | ¹H NMR (400 MHz, DMSO-d6) mixture of rotamers δ 9.99 (d, J = 6.1 Hz, 1H), 8.80 (dd, J = 7.6, 1.8 Hz, 1H), 8.43 (t, J = 2.6 Hz, 1H), 8.24 (dt, J = 8.3, 2.1 Hz, 1H), 7.91 (dt, J = 5.0, 2.6 Hz, 1H), 7.52 (t, J = 7.2 Hz, 1H), 7.37 (td, J = 8.1, 2.0 Hz, 1H), 7.18 (d, J = 4.3 Hz, 1H), 7.08 (d, J = 7.9 Hz, 1H), 4.83 (s, 0.5H), 4.69 (dd, J = 13.1, 4.1 Hz, 0.5H), 4.28 − 4.19 (m, 0.5H), 4.05 (dd, J = 13.3, 4.4 Hz, 0.5H), 3.93 (s, 3H), 3.50 − 3.41 (m, 0.5H), 2.92 (t, J = 12.6 Hz, 1H), 2.80 − 2.72 (m, 0.5H), 2.12 − 1.96 (m, 5H), 1.90 − 1.78 (m, 0.5H), 1.75 − 1.64 (m, 1.5H), 1.30 (d, J = 6.8 Hz, 1.5H), 1.16 (d, J = 6.9 Hz, 1.5H); LCMS (Method D): t_{R} 3.46 min, 100%, MS (ESI) 452.1 (M + H)⁺ |
| 00247 | 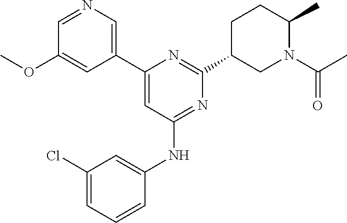 [SMILES: COc1cncc(-c2cc(Nc3cccc(Cl)c3)nc([C@@H]3CC[C@@H](C)N(C(C)=O)C3)n2)c1] (+/−)-trans-1-(5-(4-((3- | ¹H NMR (400 MHz, DMSO-d6) mixture of rotamers δ 10.02 − 9.82 (m, 1H), 8.79 (d, J = 1.8 Hz, 1H), 8.42 (d, J = 2.9 Hz, 1H), 8.14 (t, J = 2.0 Hz, 1H), 8.04 − 7.79 (m, 1H), 7.54 (d, J = 8.3 Hz, 1H), 7.36 (t, J = 8.1 Hz, 1H), 7.15 (s, 1H), 7.10 − 7.03 (m, 1H), 5.44 − 5.10 (m, 0.3H), 4.75 − 4.40 (m, 1.4H), 4.07 − 3.90 (m, 3.3H), 3.65 − 3.48 (m, 0.7H), 3.20 − 2.99 (m, 1.3H), 2.46 − 2.37 (m, 1H), 2.15 − 2.05 (m, 1H), 1.93 − 1.81 (m, 4H), 1.43 (d, J = 13.3 Hz, 1H), 1.26 − 1.10 (m, 3H); LCMS (Method D): t_{R} 3.57 min, 99%, MS (ESI) 452.1 (M + H)⁺ |
| | chlorophenyl)amino)-6-(5- | |
| | methoxypyridin-3-yl)pyrimidin-2-yl)- | |
| | 2-methylpiperidin-1-yl)ethan-1-one | |
| 00248 | 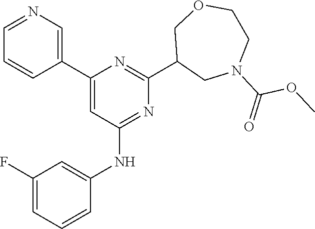 [SMILES: COC(=O)N1CCOCC(c2nc(Nc3cccc(F)c3)cc(-c3cccnc3)n2)C1] | ¹H NMR (400 MHz, DMSO-d6) mixture of rotamers δ 10.01 (d, J = 2.9 Hz, 1H), 9.20 (d, J = 2.2 Hz, 1H), 8.71 (dd, J = 4.9, 1.6 Hz, 1H), 8.38 (d, J = 8.0 Hz, 1H), 7.88 (t, J = 12.3 Hz, 1H), 7.58 (dd, J = 7.9, 4.8 Hz, 1H), 7.45 − 7.33 (m, 2H), 7.18 (s, 1H), 6.90 − 6.81 (m, 1H), 4.29 − 4.03 (m, 3H), 3.85 − 3.66 (m, 4H), 3.60 (d, J = 18.5 Hz, 3H), 3.47 − 3.36 (m, 2H); LCMS (Method D): t_{R} 3.18 min, 100%, MS (ESI) 424.2 (M + H)⁺ |
| | methyl 6-(4-((3- | |
| | fluorophenyl)amino)-6-(pyridin-3- | |
| | yl)pyrimidin-2-yl)-1,4-oxazepane-4- | |
| | carboxylate | |
| 00249 | 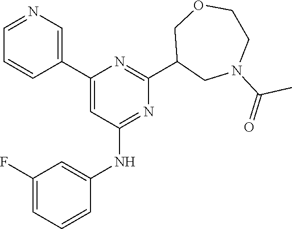 [SMILES: CC(=O)N1CCOCC(c2nc(Nc3cccc(F)c3)cc(-c3cccnc3)n2)C1] 1-(6-(4-((3-fluorophenyl)amino)-6- | ¹H NMR (400 MHz, DMSO-d6) mixture of rotamers δ 10.02 (d, J = 15.0 Hz, 1H), 9.21 (dd, J = 5.8, 2.3 Hz, 1H), 8.71 (dt, J = 4.4, 2.0 Hz, 1H), 8.39 (ddt, J = 8.1, 3.9, 2.0 Hz, 1H), 7.88 (dq, J = 12.2, 2.4 Hz, 1H), 7.61 − 7.54 (m, 1H), 7.45 − 7.33 (m, 2H), 7.18 (d, J = 7.1 Hz, 1H), 6.90 − 6.81 (m, 1H), 4.47 (dd, J = 13.5, 5.3 Hz, 0.5H), 4.24 4.07 (m, 2H), 4.07 − 3.87 (m, 1.5H), 3.85 − 3.76 (m, 1.5H), 3.76 − 3.68 (m, 1H), 3.60 − 3.41 (m, 1.5H), 3.40 − 3.27 (m, 1H), 2.11 (s, 1.5H), 2.06 (s, 1.5H); LCMS (Method D): t_{R} 3.25 min, 100%, MS (ESI) 408.2 (M + H)⁺ |
| | (pyridin-3-yl)pyrimidin-2-yl)-1,4- | |
| | oxazepan-4-yl)ethan-1-one | |
| 00250 and 00251 | 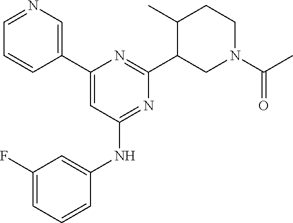 [SMILES: CC(=O)N1CCC(C)C(c2nc(Nc3cccc(F)c3)cc(-c3cccnc3)n2)C1] 1-(3-(4-((3-fluorophenyl)amino)-6- (pyridin-3-yl)pyrimidin-2-yl)-4- methylpiperidin-1-yl)ethan-1-one | 00250 single diastereoisomer, relative stereochemistry unknown: ¹H NMR (400 MHz, DMSO-d6) mixture of rotamers δ 10.09 − 9.96 (m, 1H), 9.30 − 9.15 (m, 1H), 8.79 − 8.67 (m, 1H), 8.47 − 8.34 (m, 1H), 7.93 − 7.81 (m, 1H), 7.62 − 7.53 (m, 1H), 7.49 − 7.33 (m, 2H), 7.20 (d, J = 4.9 Hz, 1H), 6.85 (t, J = 8.5 Hz, 1H), 4.66 − 4.41 (m, 1H), 4.04 − 3.86 (m, 1H), 3.15 (t, J = 12.6 Hz, 0.5H), 2.80 − 2.54 (m, 1H), 2.48 − 2.39 (m, 0.5H), 2.23 − 2.10 (m, 1H), 2.04 (d, J = 6.9 Hz, 3H), 1.87 − 1.73 (m, 1H), 1.40 − 1.11 (m, 2H), 0.90 − 0.78 (m, 3H); LCMS (Method D): t_{R} 3.21 min, 98%, MS (ESI) 406.1 (M + H)⁺ 00251 single diastereoisomer, relative |
| | | stereochemistry unknown: ¹H NMR |
| | | (400 MHz, DMSO-d6) mixture of |
| | | rotamers δ 10.00 (s, 1H), 9.35 − 9.00 |
| | | (m, 1H), 8.84 − 8.57 (m, 1H), 8.49 − |
| | | 8.24 (m, 1H), 7.95 − 7.73 (m, 1H), |
| | | 7.67 − 7.04 (m, 4H), 6.92 − 6.70 (m, |
| | | 1H), 4.55 − 4.20 (m, 0.5H), 4.02 − 3.70 |
| | | (m, 1.5H), 3.66 − 2.89 (m, 3H), 2.22 − |
| | | 1.51 (m, 6H), 0.96 − 0.65 (m, 3H); |
| | | LCMS (Method D): t_{R} 3.30 min, 97%, |
| | | MS (ESI) 406.1 (M + H)⁺ |
| 00252 | 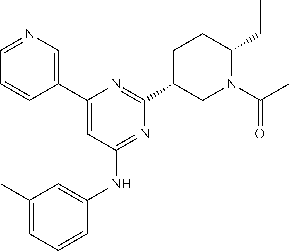 [SMILES: CC[C@H]1CC[C@@H](c2nc(Nc3cccc(C)c3)cc(-c3cccnc3)n2)CN1C(C)=O] | ¹H NMR (400 MHz, DMSO-d6) mixture of rotamers δ 9.82 − 9.60 (m, 1H), 9.25 − 9.10 (m, 1H), 8.76 − 8.63 (m, 1H), 8.43 − 8.28 (m, 1H), 7.81 − 7.68 (m, 1H), 7.62 − 7.52 (m, 1H), 7.52 − 7.40 (m, 1H), 7.30 − 7.18 (m, 1H), 7.18 − 7.09 (m, 1H), 6.91 − 6.81 (m, 1H), 4.80 − 4.69 (m, 0.5H), 4.68 − 4.56 (m, 0.5H), 4.07 − 3.97 (m, 0.5H), 3.96 − 3.85 (m, 0.5H), 3.47 − 3.36 (m, 1H), 2.94 − 2.79 (m, 1H), 2.78 − 2.64 (m, 0.5H), 2.38 − 2.25 (m, 3H), 2.09 − 2.06 (m, 3H), 2.05 − 1.48 (m, 6H), 0.95 − 0.73 (m, 3H); LCMS (Method B): t_{R} 3.12 min, 100%, MS (ESI) 416.2 (M + H)⁺ |
| | (+/−)-cis-1-(2-ethyl-5-(4-(pyridin-3- | |
| | yl)-6-(m-tolylamino)pyrimidin-2- | |
| | yl)piperidin-1-yl)ethan-1-one | |
| 00253 | 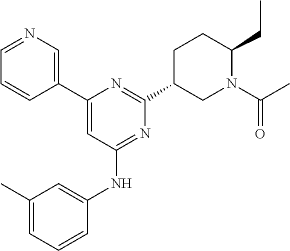 [SMILES: CC[C@@H]1CC[C@@H](c2nc(Nc3cccc(C)c3)cc(-c3cccnc3)n2)CN1C(C)=O] | ¹H NMR (400 MHz, DMSO-d6) mixture of rotamers δ 9.75 − 9.51 (m, 1H), 9.19 (s, 1H), 8.74 − 8.66 (m, 1H), 8.46 − 8.28 (m, 1H), 7.67 − 7.60 (m, 1H), 7.60 − 7.52 (m, 1H), 7.52 − 7.44 (m, 1H), 7.26 − 7.19 (m, 1H), 7.17 − 7.05 (m, 1H), 6.85 (d, J = 7.4 Hz, 1H), 5.20 (br s, 0.2H) 4.63 − 4.31 (m, 1.5H), 3.92 − 3.70 (br s, 0.2H) 3.17 − 2.92 (m, 1.5H), 2.46 − 2.36 (m, 1H), 2.32 (s, 3H), 2.13 − 1.96 (m, 1.5H), 1.95 − 1.44 (m, 7H), 0.91 − 0.70 (m, 3H); LCMS (Method B): t_{R} 3.16 min, 98%, MS (ESI) 416.2 (M + H)⁺ |
| | (+/−)-trans-1-(2-ethyl-5-(4-(pyridin- | |
| | 3-yl)-6-(m-tolylamino)pyrimidin-2- | |
| | yl)piperidin-1-yl)ethan-1-one | |
| 00254 | 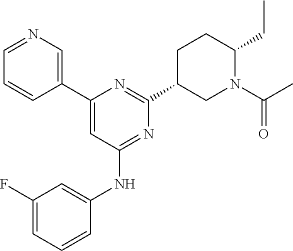 [SMILES: CC[C@H]1CC[C@@H](c2nc(Nc3cccc(F)c3)cc(-c3cccnc3)n2)CN1C(C)=O] | ¹H NMR (400 MHz, DMSO-d6) mixture of rotamers δ 10.06 − 9.94 (m, 1H), 9.26 − 9.13 (m, 1H), 8.75 − 8.67 (m, 1H), 8.44 − 8.33 (m, 1H), 8.04 − 7.91 (m, 1H), 7.62 − 7.53 (m, 1H), 7.42 − 7.33 (m, 2H), 7.22 − 7.14 (m, 1H), 6.90 − 6.78 (m, 1H), 4.78 − 4.69 (m, 0.5H), 4.66 − 4.55 (m, 0.5H), 4.10 − 4.00 (m, 0.5H), 3.97 − 3.85 (m, 0.5H), 3.46 − 3.34 (m, 0.5H), 2.97 − 2.69 (m, 2H), 2.12 − 2.06 (m, 3H), 2.05 − 1.46 (m, 6H), 0.93 − 0.76 (m, 3H); LCMS (Method B): t_{R} 3.16 min, 99%, MS (ESI) 420.2 (M + H)⁺ |
| | (+/−)-cis-1-(2-ethyl-5-(4-((3- | |
| | fluorophenyl)amino)-6-(pyridin-3- | |
| | yl)pyrimidin-2-yl)piperidin-1- | |
| | yl)ethan-1-one | |
| 00255 | 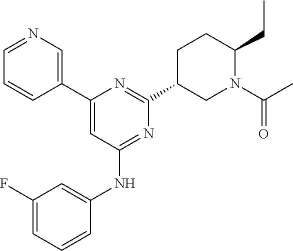 [SMILES: CC[C@@H]1CC[C@@H](c2nc(Nc3cccc(F)c3)cc(-c3cccnc3)n2)CN1C(C)=O] | ¹H NMR (400 MHz, DMSO-d6) mixture of rotamers δ 10.10 − 9.74 (m, 1H), 9.31 − 9.13 (m, 1H), 8.79 − 8.64 (m, 1H), 8.55 − 8.32 (m, 1H), 8.00 − 7.79 (m, 1H), 7.65 − 7.53 (m, 1H), 7.50 − 7.29 (m, 2H), 7.24 − 7.08 (m, 1H), 6.96 − 6.75 (m, 1H), 5.36 − 5.07 (m, 0.2H), 4.61 − 4.27 (m, 1.6H), 3.90 − 3.74 (m, 0.3H), 3.59 − 3.46 (m, 0.8H), 3.21 − 3.09 (m, 1.3H), 2.48 − 2.38 (m, 0.8H), 2.18 − 1.96 (m, 1.5H), 1.96 − 1.39 (m, 7.5H), 0.97 −0.63 (m, 3H); LCMS (Method B): t_{R} 3.20 min, 98%, MS (ESI) 420.2 (M + H)⁺ |
| | (+/−)-trans-1-(2-ethyl-5-(4-((3- | |
| | fluorophenyl)amino)-6-(pyridin-3- | |
| | yl)pyrimidin-2-yl)piperidin-1- | |
| | yl)ethan-1-one | |
| 00256 | 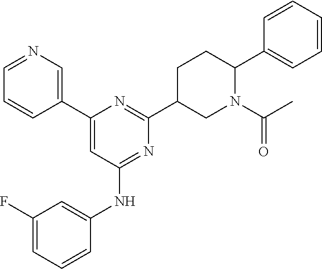 [SMILES: CC(=O)N1CC(c2nc(Nc3cccc(F)c3)cc(-c3cccnc3)n2)CCC1c1ccccc1] | ¹H NMR (400 MHz, DMSO-d6) mixture of rotamers δ 9.96 (d, J = 8.7 Hz, 1H), 9.14 (dd, J = 6.5, 2.2 Hz, 1H), 8.69 (dd, J = 4.7, 1.6 Hz, 1H), 8.37 − 8.26 (m, 1H), 7.79 − 7.64 (m, 1H), 7.55 (dd, J = 8.0, 4.8 Hz, 1H), 7.47 − 7.21 (m, 7H), 7.14 (d, J = 3.4 Hz, 1H), 6.87 − 6.76 (m, 1H), 5.87 (d, J = 5.0 Hz, 0.5H), 5.29 (s, 0.5H), 4.83 (d, J = 13.1 Hz, 0.5H), 4.18 (d, J = 14.4 Hz, 0.5H), 3.15 − 3.02 (m, 0.5H), 2.93 (s, 0.5H), 2.81 (d, J = 12.5 Hz, 0.5H), 2.57 (d, J = 11.8 Hz, 0.5H), 2.25 (s, 2H), 2.10 (d, J = 10.3 Hz, 2H), 2.01 − 1.86 (m, 1H), 1.72 (t, J = 12.6 Hz, 1H); LCMS (Method B): t_{R} 3.40 min, 99%, MS (ESI) 468.2 (M + H)⁺ |
| | 1-(5-(4-((3-fluorophenyl)amino)-6- | |
| | (pyridin-3-yl)pyrimidin-2-yl)-2- | |
| | phenylpiperidin-1-yl)ethan-1-one | |
| 00257 | 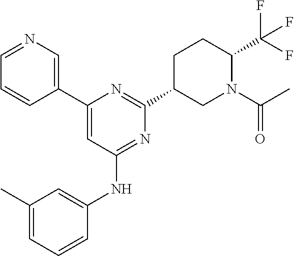 [SMILES: CC(=O)N1C[C@H](c2nc(Nc3cccc(C)c3)cc(-c3cccnc3)n2)CC[C@@H]1C(F)(F)F] | ¹H NMR (400 MHz, DMSO-d6) mixture of rotamers δ 9.77 − 9.69 (m, 1H), 9.22 − 9.14 (m, 1H), 8.74 − 8.64 (m, 1H), 8.40 − 8.31 (m, 1H), 7.74 − 7.41 (m, 3H), 7.26 − 7.18 (m, 1H), 7.18 − 7.09 (m, 1H), 6.90 − 6.81 (m, 1H), 5.39 − 5.22 (m, 0.6H), 4.95 − 4.82 (m, 0.6H), 4.28 − 4.15 (m, 0.6H), 3.58 − 3.46 (m, 0.7H), 3.08 − 2.96 (m, 0.7H), 2.96 − 2.77 (m, 0.7H), 2.34 − 2.27 (m, 3H), 2.24 − 1.84 (m, 7H); LCMS (Method B): t_{R} 3.16 min, 98%, MS (ESI) 456.2 (M + H)⁺ |
| | (+/−)-cis-1-(5-(4-(pyridin-3-yl)-6-(m- | |
| | tolylamino)pyrimidin-2-yl)-2- | |
| | (trifluoromethyl)piperidin-1- | |
| | yl)ethan-1-one | |
| 00258 | 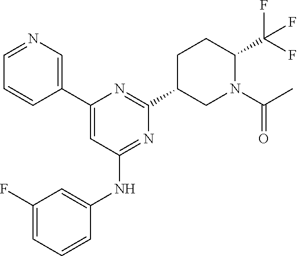 [SMILES: CC(=O)N1C[C@H](c2nc(Nc3cccc(F)c3)cc(-c3cccnc3)n2)CC[C@@H]1C(F)(F)F] | ¹H NMR (400 MHz, DMSO-d6) mixture of rotamers δ 10.08 − 9.95 (m, 7H), 9.23 − 9.17 (m, 1H), 8.74 − 8.68 (m, 1H), 8.42 − 8.34 (m, 1H), 7.97 − 7.88 (m, 1H), 7.61 − 7.55 (m, 1H), 7.41 − 7.32 (m, 2H), 7.21 − 7.15 (m, 1H), 6.88 − 6.83 (m, 1H), 5.39 − 5.23 (m, 0.6H), 4.97 − 4.77 (m, 0.6H), 4.32 − 4.19 (m, 0.6H), 3.58 − 3.45 (m, 0.7H), 3.13 − 2.99 (m, 0.7H), 2.99 − 2.81 (m, 0.6H), 2.23 − 1.83 (m, 7H); LCMS (Method B): t_{R} 3.16 min, 98%, MS (ESI) 460.2 (M + H)⁺ |
| | (+/−)-cis-1-1-(5-(4-((3- | |
| | fluorophenyl)amino)-6-(pyridin-3- | |
| | yl)pyrimidin-2-yl)-2- | |
| | (trifluoromethyl)piperidin-1- | |
| | yl)ethan-1-one | |
| 00259 | 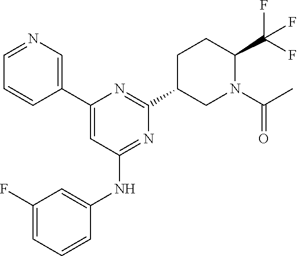 [SMILES: CC(=O)N1C[C@H](c2nc(Nc3cccc(F)c3)cc(-c3cccnc3)n2)CC[C@H]1C(F)(F)F] | 1H NMR (400 MHz, DMSO-d6) mixture of rotamers δ 10.07 − 9.86 (m, 1H), 9.27 − 9.16 (m, 1H), 8.77 − 8.66 (m, 1H), 8.44 − 8.31 (m, 1H), 7.93 − 7.74 (m, 1H), 7.63 − 7.51 (m, 1H), 7.48 − 7.31 (m, 2H), 7.24 − 7.11 (m, 1H), 6.94 − 6.80 (m, 1H), 6.70 − 6.13 (m, 1H), 5.41 − 5.24 (m, 0.2H), 4.98 − 4.78 (m, 0.1H), 4.77 − 4.46 (m, 1.2H), 4.41 − 4.07 (m, 0.2H), 3.85 − 3.49 (m, 0.7H), 3.27 − 2.93 (m, 1.4H), 2.47 − 2.37 (m, 1H), 2.24 − 1.69 (m, 6H); LCMS (Method B): t_{R} 3.33 min, 98%, MS (ESI) 460.2 (M + H)⁺ |
| | (+/−)-trans-1-(5-(4-((3- | |
| | fluorophenyl)amino)-6-(pyridin-3- | |
| | yl)pyrimidin-2-yl)-2- | |
| | (trifluoromethyl)piperidin-1- | |
| | yl)ethan-1-one | |
| 00260 | 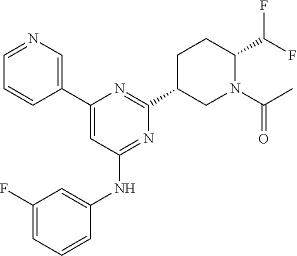 [SMILES: CC(=O)N1C[C@H](c2nc(Nc3cccc(F)c3)cc(-c3cccnc3)n2)CC[C@@H]1C(F)F] | ¹H NMR (400 MHz, DMSO-d6) mixture of rotamers δ 10.07 − 9.96 (m, 1H), 9.27 − 9.18 (m, 1H), 8.76 − 8.68 (m, 1H), 8.46 − 8.33 (m, 1H), 7.98 − 7.80 (m, 1H), 7.61 − 7.54 (m, 1H), 7.46 − 7.31 (m, 2H), 7.19 (d, J = 2.9 Hz, 1H), 6.91 − 6.79 (m, 1H), 6.65 − 6.13 (m, 1H), 4.98 − 4.81 (m, 1H), 4.39 − 4.21 (m, 1H), 4.12 − 4.04 (m, 0.1H), 3.91 − 3.75 (m, 0.1H), 3.57 − 3.44 (m, 0.5H), 3.08 − 2.75 (m, 1.4H), 2.26 − 1.89 (m, 6.4H), 1.86 − 1.70 (m, 0.6H); LCMS (Method D): t_{R} 3.27 min, 96%, MS (ESI) 442.1 (M + H)⁺ |
| | (+/−)-cis-1-(2-(difluoromethyl)-5-(4- | |
| | ((3-fluorophenyl)amino)-6-(pyridin- | |
| | 3-yl)pyrimidin-2-yl)piperidin-1- | |
| | yl)ethan-1-one | |
| 00261 | 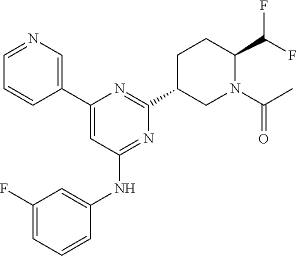 [SMILES: CC(=O)N1C[C@H](c2nc(Nc3cccc(F)c3)cc(-c3cccnc3)n2)CC[C@H]1C(F)F] | ¹H NMR (400 MHz, DMSO-d6) mixture of rotamers δ 10.07 − 9.86 (m, 1H), 1H), 8.44 − 8.31 (m, 1H), 7.93 − 7.74 (m, 1H), 7.63 − 7.51 (m, 1H), 7.48 − 7.31 (m, 2H), 7.24 − 7.11 (m, 1H), 6.94 − 6.80 (m, 1H), 6.70 − 6.13 (m, 1H), 5.41 − 5.24 (m, 0H), 4.98 − 4.78 (m, 0H), 4.77 − 4.46 (m, 1H), 4.41 − 4.07 (m, 0H), 3.85 − 3.49 (m, 1H), 2.47 − 2.37 (m, 1H), 2.24 − 1.69 (m, 6H); LCMS (Method B): t_{R} 3.38 min, 95%, MS (ESI) 442.1 (M + H)⁺ |
| | (+/−)-trans-1-(2-(difluoromethyl)-5- | |
| | (4-((3-fluorophenyl)amino)-6- | |
| | (pyridin-3-yl)pyrimidin-2- | |
| | yl)piperidin-1-yl)ethan-1-one | |
| 00262 | 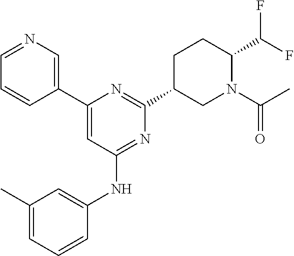 [SMILES: CC(=O)N1C[C@H](c2nc(Nc3cccc(C)c3)cc(-c3cccnc3)n2)CC[C@@H]1C(F)F] | ¹H NMR (400 MHz, DMSO-d6) mixture of rotamers δ 9.75 − 9.65 (m, 1H), 9.25 − 9.14 (m, 1H), 8.76 − 8.65 (m, 1H), 8.43 − 8.33 (m, 1H), 7.75 − 7.44 (m, 3H), 7.27 − 7.19 (m, 1H), 7.18 − 7.12 (m, 1H), 6.90 − 6.83 (m, 1H), 6.64 − 6.14 (m, 1H), 5.02 − 4.81 (m, 1H), 4.38 − 4.16 (m, 1H), 3.63 − 3.46 (m, 0.5H), 3.04 − 2.87 (m, 1H), 2.84 − 2.72 (m, 0.5H), 2.37 − 2.29 (m, 3H), 2.24 − 1.88 (m, 6.5H), 1.83 − 1.69 (m, 0.5H); LCMS (Method D): t_{R} 3.33 min, 100%, MS (ESI) 438.2 (M + H)⁺ |
| | (+/−)-cis-1-(2-(difluoromethyl)-5-(4- | |
| | (pyridin-3-yl)-6-(m- | |
| | tolylamino)pyrimidin-2-yl)piperidin- | |
| | 1-yl)ethan-1-one | |
| 00263 | 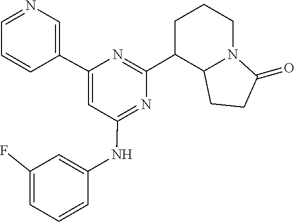 [SMILES: O=C1CCC2C(c3nc(Nc4cccc(F)c4)cc(-c4cccnc4)n3)CCCN12] (+/−)-8-(4-((3-fluorophenyl)amino)- 6-(pyridin-3-yl)pyrimidin-2- yl)hexahydroindolizin-3(2H)-one | ¹H NMR (400 MHz, chloroform-d) single diastereoisomer, absolute stereochemistry unknown δ 9.17 (d, J = 2.4 Hz, 1H), 8.71 (dd, J = 4.9, 1.7 Hz, 1H), 8.32 (dt, J = 8.1, 2.0 Hz, 1H), 7.43 (dd, J = 7.9, 4.7 Hz, 1H), 7.40 (dt, J = 10.5, 2.1 Hz, 1H), 7.36 (td, J = 8.1, 6.3 Hz, 1H), 7.16 (dd, J = 7.8, 2.0 Hz, 1H), 7.01 (s, 1H), 7.00 (s, 1H), 6.89 (td, J = 8.1, 2.4 Hz, 1H), 4.25 (dd, J = 13.3, 4.8 Hz, 1H), 4.00 (dt, J = 10.3, 7.1 Hz, 1H), 2.76 (td, J = 13.3, 2.9 Hz, 1H), 2.69 (td, J = 11.0, 2.7 Hz, 1H), 2.44 (dt, J = 17.0, 5.0 Hz, 1H), 2.39 (ddd, J = 17.0, 8.5, 1.0 Hz, 1H), 2.29- 2.14 (m, 2H), 1.91-1.71 (m, 3H), 1.66-1.52 (m, 1H); LCMS (Method D): t_{R} 3.45 min, 100%, MS (ESI) 404.2 (M + H)⁺ |

### Example 27: Synthesis of N-(2-(2-(2-acetamidoethoxy)ethoxy)ethyl)-4-(2-(1-acetylpiperidin-3-yl)-6-((3-fluorophenyl)amino)pyrimidin-4-yl)benzamide (00264)

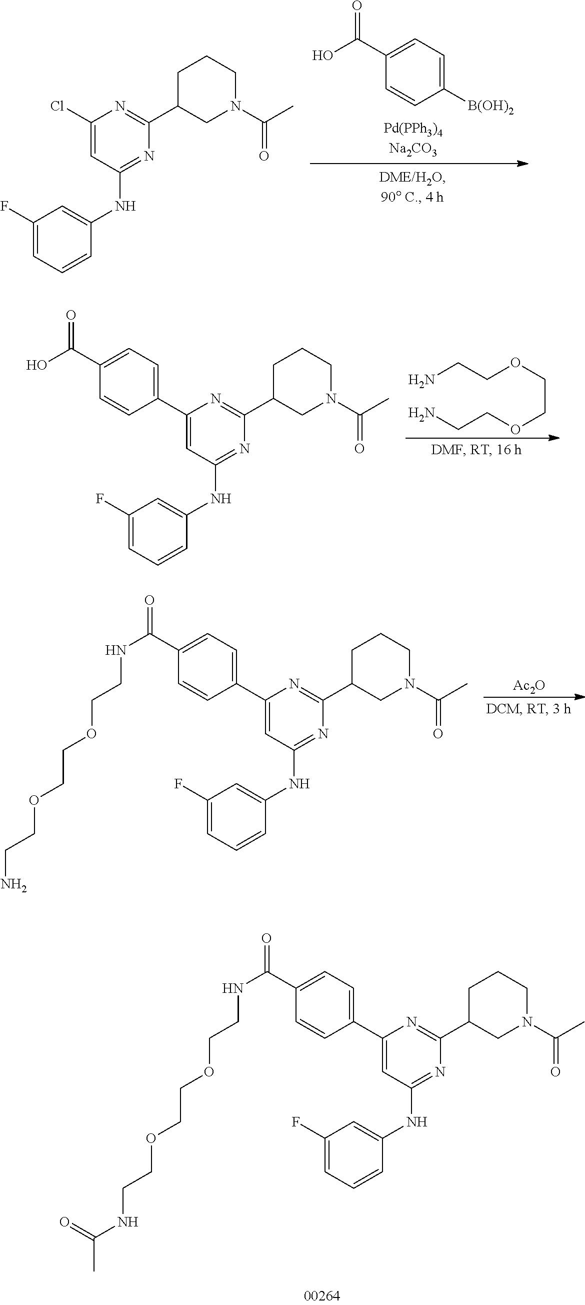

Under argon atmosphere, a microwave vial was charged with 1-(3-(4-chloro-6-((3-fluorophenyl)amino)pyrimidin-2-yl)piperidin-1-yl)ethan-1-one (380 mg, 1.09 mmol), 4-carboxyphenylboronic acid (724 mg, 4.36 mmol), tetrakis(triphenylphosphine)palladium (126 mg, 0.109 mmol) and sodium carbonate (346 mg, 3.27 mmol) in 1,2-dimethoxyethane (12 mL) and water (4 mL). The mixture was heated in at 90° C. for 4 hours, poured into water and extracted with ethyl acetate twice. The combined organic layers were washed with brine, dried with sodium sulfate and concentrated in vacuo to afford a yellow gum. The residue was purified by reversed phase chromatography (method B) to afford 4-(2-(1-acetylpiperidin-3-yl)-6-((3-fluorophenyl)amino)pyrimidin-4-yl)benzoic acid (52 mg, 10%) as a white solid. ¹H-NMR (400 MHz, DMSO-d6) δ 9.99 (d, J=10.7 Hz, 1H), 8.21-8.04 (m, 4H), 7.90 (t, J=12.5 Hz, 1H), 7.47-7.33 (m, 2H), 7.20 (d, J=3.8 Hz, 1H), 6.84 (t, J=8.4 Hz, 1H), 4.77 (d, J=12.2 Hz, 0.5H), 4.25 (d, J=12.9 Hz, 0.5H), 4.16 (dd, J=13.7, 3.9 Hz, 0.5H), 3.87 (d, J=13.6 Hz, 0.5H), 3.50 (dd, J=13.4, 10.3 Hz, 0.5H), 3.08 (t, J=12.6 Hz, 0.5H), 2.96 (td, J=10.4, 5.2 Hz, 1H), 2.90-2.66 (m, 2H), 2.30-2.17 (m, 1H), 2.05 (s, 3H), 1.93-1.73 (m, 2H), 1.70-1.38 (m, 1H); LCMS (Method C): t_{R} 1.72 min, 100%, MS (ESI) 435.1 (M+H)⁺. To a solution of 4-(2-(1-acetylpiperidin-3-yl)-6-((3-fluorophenyl)amino)pyrimidin-4-yl)benzoic acid (50 mg, 0.12 mmol) and 2,2′-(ethane-1,2-diylbis(oxy))bis(ethan-1-amine) (342 mg, 2.30 mmol) in N, N-dimethylformamide (3 mL) was added 1-[bis(dimethylamino)methylene]-1H-1,2,3-triazolo[4,5-b]pyridinium 3-oxid hexafluorophosphate (104 mg, 0.28 mmol) and the mixture was stirred at room temperature for 16 hours. The mixture was concentrated in vacuo and purified by reversed phase chromatography (method B) to afford a white solid. The crude was further purified with SCX (ion-exchange) chromatography (washed with methanol and eluted with 3.5M ammonia in methanol) to afford 4-(2-(1-acetylpiperidin-3-yl)-6-((3-fluorophenyl)amino)pyrimidin-4-yl)-N-(2-(2-(2-aminoethoxy)ethoxy)ethyl)benzamide (35 mg, 54%) as a white solid. LCMS (Method C): t_{R} 1.89 min, 100%, MS (ESI) 565.3 (M+H)⁺. To a solution of 4-(2-(1-acetylpiperidin-3-yl)-6-((3-fluorophenyl)amino)pyrimidin-4-yl)-N-(2-(2-(2-aminoethoxy)ethoxy)ethyl)benzamide (15 mg, 0.03 mmol) in dichloromethane (1 mL) was added acetic anhydride (10 μL, 0.11 mmol) and the mixture was stirred at room temperature for 3 hours. The mixture was concentrated and lyophilized to afford N-(2-(2-(2-acetamidoethoxy)ethoxy)ethyl)-4-(2-(1-acetylpiperidin-3-yl)-6-((3-fluorophenyl)amino)pyrimidin-4-yl)benzamide (12 mg, 74%) as a white solid. ¹H-NMR (400 MHz, DMSO-d6) mixture of rotamers δ 9.96 (d, J=10.6 Hz, 1H), 8.65 (t, J=5.5 Hz, 1H), 8.16-8.08 (m, 2H), 8.00 (d, J=8.2 Hz, 2H), 7.95-7.82 (m, 2H), 7.46-7.33 (m, 2H), 7.19 (d, J=4.2 Hz, 1H), 6.84 (t, J=8.5 Hz, 1H), 4.76 (d, J=12.2 Hz, 0.5H), 4.23 (d, J=12.9 Hz, 0.5H), 4.15 (d, J=15.2 Hz, 0.5H), 3.86 (d, J=13.6 Hz, 0.5H), 3.61-3.50 (m, 7H), 3.50-3.42 (m, 2.5H), 3.40 (t, J=5.9 Hz, 2H), 3.20-3.14 (m, 2H), 3.09 (t, J=12.5 Hz, 0.5H), 3.02-2.92 (m, 0.5H), 2.92-2.83 (m, 0.5H), 2.83-2.72 (m, 1H), 2.29-2.19 (m, 1H), 2.05 (s, 3H), 1.96-1.72 (m, 5H), 1.67-1.41 (m, 1H); LCMS (Method D): t_{R} 3.07 min, 95%, MS (ESI) 607.2 (M+H)⁺.

### Example 28: Synthesis of 1-(3-(6-((3-fluorophenyl)amino)-2-(pyridin-3-yl)pyrimidin-4-yl)piperidin-1-yl)ethan-1-one (00265)

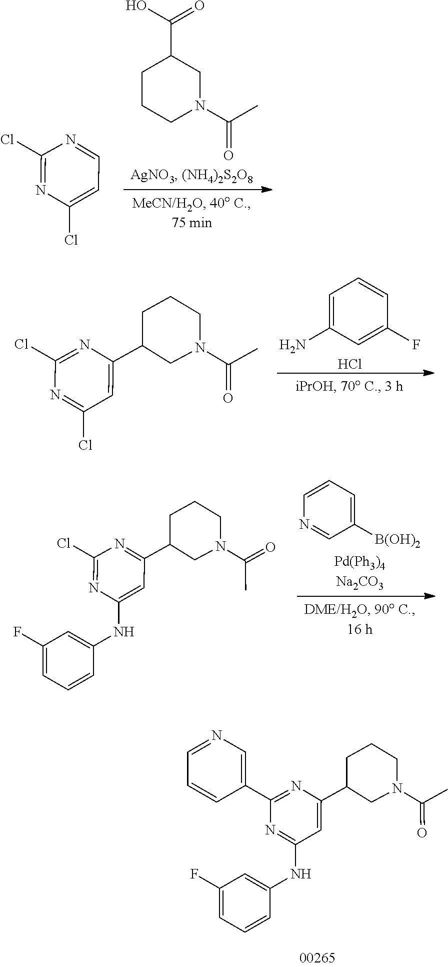

A mixture of 1-acetylpiperidine-3-carboxylic acid (3.45 g, 20.14 mmol), 2,4-dichloropyrimidine (1 g, 6.71 mmol), silver nitrate (9.12 g, 53.7 mmol), and ammonium persulfate (12.25 g, 53.7 mmol) in water (20 mL) and acetonitrile (40 mL), was stirred vigorously at 40° C. for 75 minutes. The mixture was diluted with ethyl acetate and water. After vigorous stirring for 5 minutes, the mixture was decanted and the residue was washed with ethyl acetate. The layers were separated, the organic layer was washed with brine, followed by a mixture of brine and saturated sodium hydrogen carbonate solution (1/1, v/v). The organic layer was dried with sodium sulfate, concentrated in vacuo and the residue was purified with silica column chromatography (50% to 100% ethyl acetate in n-heptane) to afford an oil, which was crystallized from diethylether to afford 1-(3-(2,6-dichloropyrimidin-4-yl)piperidin-1-yl)ethan-1-one (384 mg, 21%) as colorless crystals. ¹H-NMR (400 MHz, Chloroform-d) mixture of rotamers δ 7.21 (2×s, 1H), 4.71-4.61 (m, 0.6H), 4.58-4.50 (m, 0.4H), 4.00-3.91 (m, 0.4H), 3.86-3.76 (m, 0.6H), 3.43 (dd, J=13.4, 10.6 Hz, 0.4H), 3.23-3.09 (m, 0.6H), 2.95 (dd, J=13.0, 10.7 Hz, 0.6H), 2.88-2.66 (m, 1.4H), 2.19-2.03 (m, 4H), 1.99-1.74 (m, 2H), 1.66-1.51 (m, 1H); LCMS (Method C): t_{R} 1.79 min, 100%, MS (ESI) 274.0 (M+H)⁺. To a solution of 3-fluoroaniline (0.05 mL, 0.55 mmol) and 1-(3-(2,6-dichloropyrimidin-4-yl)piperidin-1-yl)ethan-1-one (151.3 mg, 0.55 mmol) in 2-propanol (1 mL), was added concentrated hydrochloric acid (0.14 mL, 1.66 mmol) and the mixture was stirred at 70° C. for 3 hours. The mixture was poured into a mixture of saturated sodium hydrogen carbonate solution and brine (1/1, v/v) and was extracted with ethyl acetate. The combined organic layers were dried, concentrated in vacuo and the residue was purified by reversed phase chromatography (Method B) to afford 1-(3-(2-chloro-6-((3-fluorophenyl)amino)pyrimidin-4-yl)piperidin-1-yl)ethan-1-one (28 mg, 15%) as a white solid. ¹H-NMR (400 MHz, Chloroform-d) mixture of rotamers δ 8.14 (s, 0.6H), 7.88 (s, 0.4H), 7.39-7.22 (m, 2H), 7.21-7.13 (m, 1H), 6.92-6.79 (m, 1H), 6.51 and 6.48 (2s, 1H), 4.58-4.42 (m, 1H), 3.94-3.85 (m, 0.4H), 3.82-3.72 (m, 0.6H), 3.50-3.36 (m, 0.4H) 3.24-3.13 (m, 0.6H), 3.09-2.97 (m, 0.6H), 2.79-2.58 (m, 1.4H), 2.14 and 2.13 (2×s, 3H), 2.07-1.88 (m, 1.6H), 1.86-1.72 (m, 1.4H), 1.62-1.45 (m, 1H); LCMS (Method C): t_{R} 1.98 min, 100%, MS (ESI) 349.1 (M+H)⁺. Under argon atmosphere, 1-(3-(2-chloro-6-((3-fluorophenyl)amino)pyrimidin-4-yl)piperidin-1-yl)ethan-1-one (28 mg, 0.08 mmol), pyridin-3-ylboronic acid (19.73 mg, 0.16 mmol) and sodium carbonate (34.0 mg, 0.32 mmol) were dissolved in 1,2-dimethoxyethane (2 mL) and water (0.5 mL). Next, tetrakis(triphenylphosphine)palladium(0) (4.64 mg, 4.01 μmol) was added and the mixture was stirred at 90° C. for 16 hours. Acetonitrile was added, the mixture was filtered through a nylon filter and the filtrate was concentrated in vacuo. The residue was purified by reversed phase chromatography (Method B) to afford 1-(3-(6-((3-fluorophenyl)amino)-2-(pyridin-3-yl)pyrimidin-4-yl)piperidin-1-yl)ethan-1-one (16 mg, 49%) as a white solid. ¹H-NMR (400 MHz, DMSO-d6) mixture of rotamers δ 9.95 (d, J=8.4 Hz, 1H), 9.57-9.42 (m, 1H), 8.78-8.68 (m, 1H), 8.67-8.56 (m, 1H), 7.91-7.74 (m, 1H), 7.58 (dd, J=8.1, 4.8 Hz, 1H), 7.52-7.36 (m, 2H), 6.96-6.81 (m, 1H), 6.77-6.64 (m, 1H), 4.66-4.53 (m, 0.5H), 4.40-4.24 (m, 0.5H), 4.08-3.94 (m, 0.5H), 3.91-3.79 (m, 0.5H), 3.48-3.36 (m, 0.5H), 3.20-3.05 (m, 0.5H), 2.92-2.78 (m, 1H), 2.77-2.62 (m, 1H), 2.14-1.98 (m, 4H), 1.93-1.71 (m, 2H), 1.66-1.38 (m, 1H); LCMS (Method D): t_{R} 3.32 min, 99%, MS (ESI) 392.2 (M+H)⁺.

### Example 29: Synthesis of 1-(3-(2-((3-fluorophenyl)amino)-6-(pyridin-3-yl)pyrimidin-4-yl)piperidin-1-yl)ethan-1-one (00266)

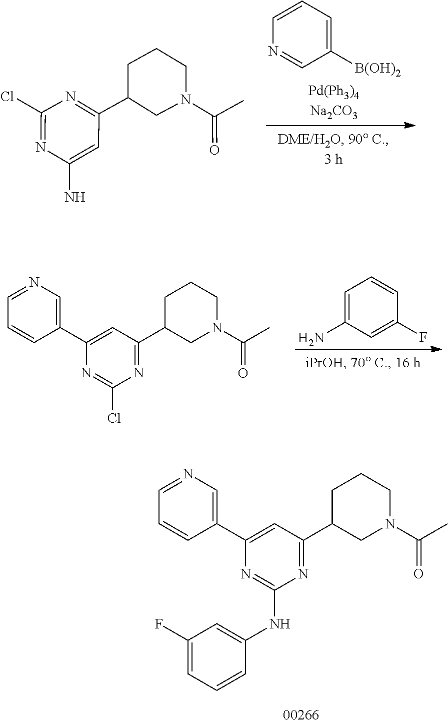 [SMILES: CC(=O)N1CCCC(c2cc(N)nc(Cl)n2)C1 ; OB(O)c1cccnc1 ; CC(=O)N1CCCC(c2cc(-c3cccnc3)nc(Cl)n2)C1 ; Nc1cccc(F)c1 ; CC(=O)N1CCCC(c2cc(-c3cccnc3)nc(Nc3cccc(F)c3)n2)C1]

Under argon atmosphere, pyridine-3-boronic acid (43.9 mg, 0.36 mmol), 1-(3-(2,6-dichloropyrimidin-4-yl)piperidin-1-yl)ethan-1-one (89 mg, 0.33 mmol), and sodium carbonate (103 mg, 0.97 mmol) were dissolved in 1,2-dimethoxyethane (3 mL) and water (1 mL). Next, tetrakis(triphenylphosphine)palladium(0) (18.76 mg, 0.02 mmol) was added and the mixture was heated at 90° C. for 3 hours. The mixture was partitioned between ethyl acetate, brine and saturated sodium bicarbonate solution. The layers were separated and the aqueous layer was extracted with ethyl acetate once. The combined organic layers were washed with brine, dried with sodium sulfate and concentrated in vacuo to afford 1-(3-(2-chloro-6-(pyridin-3-yl)pyrimidin-4-yl)piperidin-1-yl)ethan-1-one (59 mg, 57%) as a brown solid. ¹H-NMR (400 MHz, Chloroform-d) mixture of rotamers δ 9.39-9.13 (m, 1H), 8.87-8.68 (m, 1H), 8.48-8.37 (m, 1H), 7.63 (2×s, 1H), 7.52-7.44 (m, 1H), 4.69-4.53 (m, 1H), 4.07-3.97 (m, 0.4H) 3.86-3.76 (m, 0.6H), 3.57-3.48 (m, 0.4H), 3.29-3.19 (m, 0.6H), 3.19-3.11 (m, 0.6H), 2.94 (m, 1H), 2.80-2.71 (m, 0.4H), 2.22-1.98 (m, 5H), 1.98-1.79 (m, 1H), 1.70-1.53 (m, 1H); LCMS (Method C): t_{R} 1.76 min, 100%, MS (ESI) 317.1 (M+H)⁺. To a solution of 3-fluoroaniline (10.52 μL, 0.11 mmol) and 1-(3-(2-chloro-6-(pyridin-3-yl)pyrimidin-4-yl)piperidin-1-yl)ethan-1-one (29 mg, 0.09 mmol) in 2-propanol (1 mL) was added concentrated hydrochloric acid (0.02 mL, 0.28 mmol) and the mixture was stirred at 70° C. for 16 hours. Ethyl acetate and saturated sodium bicarbonate solution were added and the layers were separated. The aqueous layer was extracted with ethyl acetate once, the combined organic layers were washed with brine, dried with sodium sulfate and concentrated in vacuo. The residue was purified by reversed phase chromatography (Method B) to afford 1-(3-(2-((3-fluorophenyl)amino)-6-(pyridin-3-yl)pyrimidin-4-yl)piperidin-1-yl)ethan-1-one (6 mg, 17%) as a yellow solid. ¹H NMR (400 MHz, DMSO-d6) mixture of rotamers δ 9.96 (d, J=4.1 Hz, 1H), 9.41-9.29 (m, 1H), 8.77-8.70 (m, 1H), 8.55-8.46 (m, 1H), 7.94-7.83 (m, 1H), 7.67-7.50 (m, 3H), 7.40-7.27 (m, 1H), 6.84-6.71 (m, 1H), 4.66-4.55 (m, 0.5H), 4.50-4.37 (m, 0.5H), 4.16-4.03 (m, 0.5H), 3.92-3.81 (m, 0.5H), 3.42-3.37 (m, 0.5H), 3.16-3.03 (m, 0.5H), 2.99-2.82 (m, 1H), 2.79-2.69 (m, 0.5H), 2.64-2.50 (m, 0.5H), 2.19-2.01 (m, 4H), 1.95-1.73 (m, 2H), 1.65-1.35 (m, 1H); LCMS (Method D): t_{R} 3.30 min, 99%, MS (ESI) 392.2 (M+H)⁺.

### Example 30: Synthesis of 2-(2-(2-(2-acetamidoethoxy)ethoxy)ethoxy)-N-(4-(2-(1-acetylpiperidin-3-yl)-6-((3-fluorophenyl)amino)pyrimidin-4-yl)phenyl)acetamide (00267)

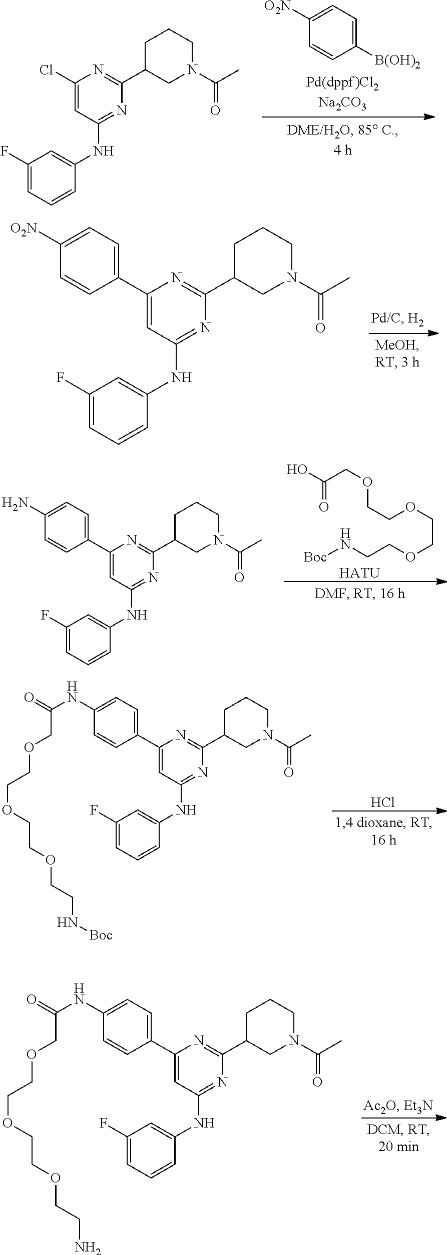

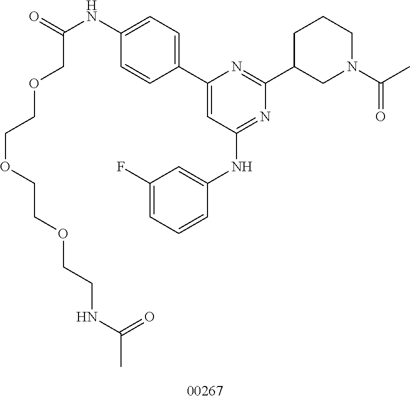 [SMILES: CC(=O)NCCOCCOCCOCC(=O)Nc1ccc(-c2cc(Nc3cccc(F)c3)nc(C3CCCN(C(C)=O)C3)n2)cc1]

Under argon atmosphere, a microwave vial was charged with 1-(3-(4-chloro-6-((3-fluorophenyl)amino)pyrimidin-2-yl)piperidin-1-yl)ethan-1-one (230 mg, 0.66 mmol), 4-nitrophenylboronic acid (220 mg, 1.32 mmol), 1,1′-bis(diphenylphosphino)ferrocenepalladium(II) dichloride (48.2 mg, 0.07 mmol) and sodium carbonate (175 mg, 1.65 mmol) in 1,2-dimethoxyethane (8 mL) and water (2 mL). The mixture was heated at 85° C. for 4 hours, poured into water and extracted with ethyl acetate twice. The combined organic layers were washed with brine, dried with sodium sulfate and concentrated in vacuo to afford 1-(3-(4-((3-fluorophenyl)amino)-6-(4-nitrophenyl)pyrimidin-2-yl)piperidin-1-yl)ethan-1-one (300 mg, 70%) as a brown gum. LCMS (Method A): t_{R} 2.10 min, 67%, MS (ESI) 436.1 (M+H)⁺. Under argon atmosphere, 1-(3-(4-((3-fluorophenyl)amino)-6-(4-nitrophenyl)pyrimidin-2-yl)piperidin-1-yl)ethan-1-one (300 mg, 0.46 mmol) and 10% palladium on carbon (100 mg, 0.10 mmol) were suspended in methanol (20 mL). Hydrogen atmosphere was introduced and the mixture was stirred at room temperature for 3 hours. The mixture was filtered through celite and the filtrate was concentrated in vacuo to afford a brown gum. The gum was purified with SCX (ion exchange) chromatography (washed with methanol and eluted with 3.5M ammonia in methanol) to afford a brown gum. The residue was purified by reversed phase chromatography (method A) to afford 1-(3-(4-(4-aminophenyl)-6-((3-fluorophenyl)amino)pyrimidin-2-yl)piperidin-1-yl)ethan-1-one (37 mg, 18%) as a yellow solid. ¹H-NMR (400 MHz, DMSO-d6) mixture of rotamers δ 9.67 (d, J=9.3 Hz, 1H), 7.88 (t, J=13.1 Hz, 1H), 7.83-7.72 (m, 2H), 7.44-7.28 (m, 3H), 6.91 (d, J=3.2 Hz, 1H), 6.78 (t, J=8.6 Hz, 1H), 6.68-6.59 (m, 2H), 5.64 (s, 2H), 4.75 (d, J=12.7 Hz, 0.5H), 4.22 (d, J=12.9 Hz, 0.5H), 4.11 (d, J=14.1 Hz, 0.5H), 3.86 (d, J=13.4 Hz, 0.5H), 3.48 (dd, J=13.5, 10.2 Hz, 0.5H), 3.06 (t, J=12.6 Hz, 0.5H), 2.93-2.84 (m, 0.5H), 2.84-2.63 (m, 1.5H), 2.21 (d, J=12.3 Hz, 1H), 2.04 (s, 3H), 1.89-1.68 (m, 2H), 1.67-1.37 (m, 1H); LCMS (Method C): t_{R} 1.99 min, 100%, MS (ESI) 406.2 (M+H)⁺. To a solution of 1-(3-(4-(4-aminophenyl)-6-((3-fluorophenyl)amino)pyrimidin-2-yl)piperidin-1-yl)ethan-1-one (35 mg, 0.09 mmol) and 2,2-dimethyl-4-oxo-3,8,11,14-tetraoxa-5-azahexadecan-16-oic acid (58.4 mg, 0.210 mmol) in N, N-dimethylformamide (3 mL) was added 1-[bis(dimethylamino)methylene]-1H-1,2,3-triazolo[4,5-b]pyridinium 3-oxid hexafluorophosphate (78.8 mg, 0.20 mmol) and the mixture was stirred at room temperature for 16 hours. The mixture was concentrated in vacuo and purified by reversed phase chromatography (method B) to afford tert-butyl (2-(2-(2-(2-((4-(2-(1-acetylpiperidin-3-yl)-6-((3-fluorophenyl)amino)pyrimidin-4-yl)phenyl)amino)-2-oxoethoxy)ethoxy)ethoxy)ethyl)carbamate (74 mg, 100%) as a colorless oil. LCMS (Method A): t_{R} 1.90 min, 89%, MS (ESI) 695.3 (M+H)⁺. To a solution of tert-butyl (2-(2-(2-(2-((4-(2-(1-acetylpiperidin-3-yl)-6-((3-fluorophenyl)amino)pyrimidin-4-yl)phenyl)amino)-2-oxoethoxy)ethoxy)ethoxy)ethyl)carbamate (60 mg, 0.09 mmol) in 1,4-dioxane (2 mL) was added 4M hydrochloric acid in 1,4-dioxane (1 mL, 4 mmol) and the mixture was stirred at room temperature for 16 hours. The mixture was concentrated in vacuo to afford N-(4-(2-(1-acetylpiperidin-3-yl)-6-((3-fluorophenyl)amino)pyrimidin-4-yl)phenyl)-2-(2-(2-(2-aminoethoxy)ethoxy)ethoxy)acetamide (55 mg, 94%) as a white gum, which was used without further purification in the next step. LCMS (Method C): t_{R} 1.94 min, 88%, MS (ESI) 595.3 (M+H)⁺. To a solution of N-(4-(2-(1-acetylpiperidin-3-yl)-6-((3-fluorophenyl)amino)pyrimidin-4-yl)phenyl)-2-(2-(2-(2-aminoethoxy)ethoxy)ethoxy)acetamide (55 mg, 0.08 mmol) and triethylamine (0.02 mL, 0.163 mmol) in dichloromethane (2 mL) was added acetic anhydride (0.01 mL, 0.122 mmol) and the mixture was stirred at room temperature for 20 minutes. The mixture was concentrated and the residue was purified by reversed phase chromatography (method B) to afford 2-(2-(2-(2-acetamidoethoxy)ethoxy)ethoxy)-N-(4-(2-(1-acetylpiperidin-3-yl)-6-((3-fluorophenyl)amino)pyrimidin-4-yl)phenyl)acetamide (8 mg, 15%) as a colorless gum. ¹H-NMR (400 MHz, DMSO-d6) δ 9.97-9.76 (m, 2H), 8.03 (dd, J=8.8, 4.3 Hz, 2H), 7.96-7.80 (m, 4H), 7.48-7.29 (m, 2H), 7.09 (d, J=4.0 Hz, 1H), 6.82 (t, J=8.5 Hz, 1H), 4.75 (d, J=12.1 Hz, 0.5H), 4.23 (d, J=13.0 Hz, 0.5H), 4.18-4.07 (m, 2.5H), 3.86 (d, J=13.3 Hz, 0.5H), 3.74-3.60 (m, 4H), 3.61-3.46 (m, 4.5H), 3.41 (t, J=5.9 Hz, 2H), 3.22-3.14 (m, 2H), 3.08 (t, J=12.2 Hz, 0.5H), 3.00-2.89 (m, 0.5H), 2.85 (t, J=11.7 Hz, 0.5H), 2.80-2.71 (m, 1H), 2.23 (d, J=12.6 Hz, 1H), 2.05 (s, 3H), 1.92-1.72 (m, 5H), 1.65-1.39 (m, 1H); LCMS (Method D): t_{R} 3.15 min, 100%, MS (ESI) 637.2 (M+H)⁺.

### Example 31: Synthesis of 7-(4-((3-fluorophenyl)amino)-6-(pyridin-3-yl)pyrimidin-2-yl)octahydro-4H-quinolizin-4-one (00268)

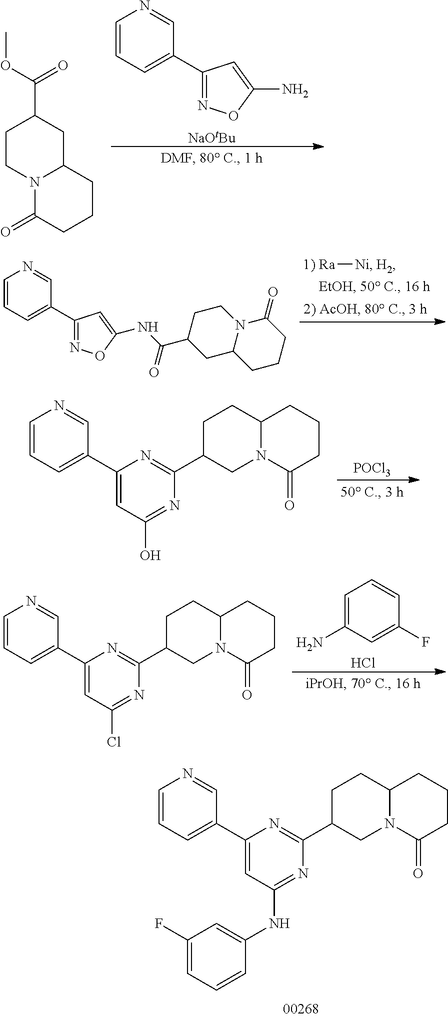

A solution of methyl 6-oxooctahydro-2H-quinolizine-3-carboxylate (410 mg, 1.94 mmol, synthesised according to WO 2014/114185, column 106), 3-(pyridin-3-yl)isoxazol-5-amine (313 mg, 1.94 mmol) and sodium tert-butoxide (560 mg, 5.82 mmol) in N, N-dimethylformamide (10 mL) was heated at 80° C. for 1 hour. The mixture was poured into saturated ammonium chloride solution and was extracted with ethyl acetate twice. The combined organic layers were washed with brine, dried with sodium sulfate and concentrated in vacuo to afford a pale yellow oil. The oil was filtered through silica (10% methanol in dichloromethane) and concentrated in vacuo to afford 6-oxo-N-(3-(pyridin-3-yl)isoxazol-5-yl)octahydro-2H-quinolizine-3-carboxamide (550 mg, 83%) as a pale yellow solid. LCMS (Method C): t_{R} 1.67 min, 42%, MS (ESI) 341.1 (M+H)⁺.

Under argon atmosphere, 6-oxo-N-(3-(pyridin-3-yl)isoxazol-5-yl)octahydro-2H-quinolizine-3-carboxamide (550 mg, 1.62 mmol) was dissolved in ethanol (20 mL) and 50% Raney®-Nickel slurry in water (catalytic amount) was added. Next, hydrogen atmosphere was introduced via a syringe and the mixture was stirred at 50° C. for 16 hours. The mixture was filtered through Celite and concentrated to afford N-(3-amino-3-(pyridin-3-yl)acryloyl)-6-oxooctahydro-2H-quinolizine-3-carboxamide as a yellow gum. This was dissolved in acetic acid (5 mL) and heated at 80° C. for 3 hours. The mixture was concentrated and coevaporated with toluene twice. The residue was purified by reversed phase chromatography (Method B) to afford 7-(4-hydroxy-6-(pyridin-3-yl)pyrimidin-2-yl)octahydro-4H-quinolizin-4-one (86 mg, 16%) as a white solid. ¹H-NMR (400 MHz, DMSO-d6) δ 12.50 (s, 1H), 9.23 (d, J=2.4 Hz, 1H), 8.72-8.60 (m, 1H), 8.45-8.34 (m, 1H), 7.51 (dd, J=8.0, 4.9 Hz, 1H), 6.88 (s, 1H), 4.88-4.72 (m, 1H), 2.78 (t, J=12.3 Hz, 1H), 2.64-2.55 (m, 1H), 2.29-2.16 (m, 2H), 2.08 (d, J=12.6 Hz, 1H), 2.03-1.94 (m, 1H), 1.87-1.70 (m, 3H), 1.68-1.56 (m, 1H), 1.56-1.44 (m, 1H), 1.42-1.28 (m, 1H); LCMS (Method C): t_{R} 1.61 min, 97%, MS (ESI) 325.1 (M+H)⁺. A solution of 7-(4-hydroxy-6-(pyridin-3-yl)pyrimidin-2-yl)octahydro-4H-quinolizin-4-one (80 mg, 0.25 mmol) in phosphorus oxychloride (4 mL, 42.9 mmol) was stirred at 50° C. for 3 hours. The mixture was concentrated, diluted with saturated sodium bicarbonate solution and extracted with ethyl acetate twice. The combined organic layers were dried over sodium sulfate and concentrated to afford 7-(4-chloro-6-(pyridin-3-yl)pyrimidin-2-yl)octahydro-4H-quinolizin-4-one (84 mg, 99%) as a yellow gum. LCMS (Method C): t_{R} 1.85 min, 99%, MS (ESI) 343.1 (M+H)⁺. To a solution of 7-(4-chloro-6-(pyridin-3-yl)pyrimidin-2-yl)octahydro-4H-quinolizin-4-one (84 mg, 0.25 mmol) and 3-fluoroaniline (0.07 ml, 0.735 mmol) in 2-propanol (4 mL) was added concentrated hydrochloric acid (0.06 mL, 0.735 mmol) and the mixture was stirred at 70° C. for 16 hours. The mixture was poured into water and extracted with ethyl acetate three times. The combined organic layers were dried over sodium sulfate and concentrated to afford a yellow gum that was purified by reversed phase chromatography (Method B) to afford 7-(4-((3-fluorophenyl)amino)-6-(pyridin-3-yl)pyrimidin-2-yl)octahydro-4H-quinolizin-4-one (32 mg, 31%) as a white solid. ¹H-NMR (400 MHz, DMSO-d6) δ 9.98 (s, 1H), 9.20 (d, J=2.2 Hz, 1H), 8.71 (dd, J=4.9, 1.6 Hz, 1H), 8.38 (dt, J=8.1, 2.0 Hz, 1H), 7.86 (dt, J=12.2, 2.2 Hz, 1H), 7.58 (dd, J=8.1, 4.9 Hz, 1H), 7.47-7.33 (m, 2H), 7.18 (s, 1H), 6.89-6.81 (m, 1H), 5.00 (dt, J=12.3, 2.8 Hz, 1H), 2.85-2.73 (m, 1H), 2.73-2.64 (m, 1H), 2.29-2.19 (m, 3H), 2.07-1.97 (m, 1H), 1.90-1.44 (m, 7H); LCMS (Method D): t_{R} 3.39 min, 100%, MS (ESI) 418.2 (M+H)⁺.

The following compound was prepared using procedures analogous to Example 31:

| Compound # | Structure and compound name | Analytical data |
|---|---|---|
| 00269 | 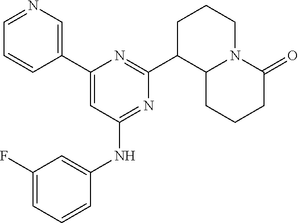 [SMILES: O=C1CCCC2C(c3nc(Nc4cccc(F)c4)cc(-c4cccnc4)n3)CCCN12] | ¹H NMR (400 MHz, DMSO-d6) δ 10.00 (s, 1H), 9.20 (d, J = 2.2 Hz, 1H), 8.71 (dd, J = 4.7, 1.6 Hz, 1H), 8.38 (dt, J = 8.3, 2.1 Hz, 1H), 7.84 (dt, J = 12.1, 2.3 Hz, 1H), 7.58 (dd, J = 8.0, 4.8 Hz, 1H), 7.41 (dt, J = 22.9, 8.2 Hz, 2H), 7.18 (s, 1H), 6.92 − 6.78 (m, 1H), 4.71 (d, J = 12.9 Hz, 1H), 3.86 − 3.72 (m, 1H), 2.84 − 2.75 (m, 1H), 2.55 (d, J = 6.3 Hz, 1H), 2.23 (d, J = 6.3 Hz, 2H), 2.05 (d, J = 15.7 Hz, 1H), 1.99 − 1.86 (m, 2H), 1.82 − 1.68 (m, 3H), 1.60 − 1.34 (m, 2H); LCMS (Method D): t_{R} 3.23 min, 93%, MS (ESI) 418.2 (M + H)⁺ |
| | 9-(4-((3-fluorophenyl)amino)-6- | |
| | (pyridin-3-yl)pyrimidin-2- | |
| | yl)octahydro-4H-quinolizin-4-one | |

### Example 32: Synthesis of (R)-1-(3-(4-((3-fluorophenyl)amino)-6-(5-methoxypyridin-3-yl)pyrimidin-2-yl)piperidin-1-yl)ethan-1-one (00270)

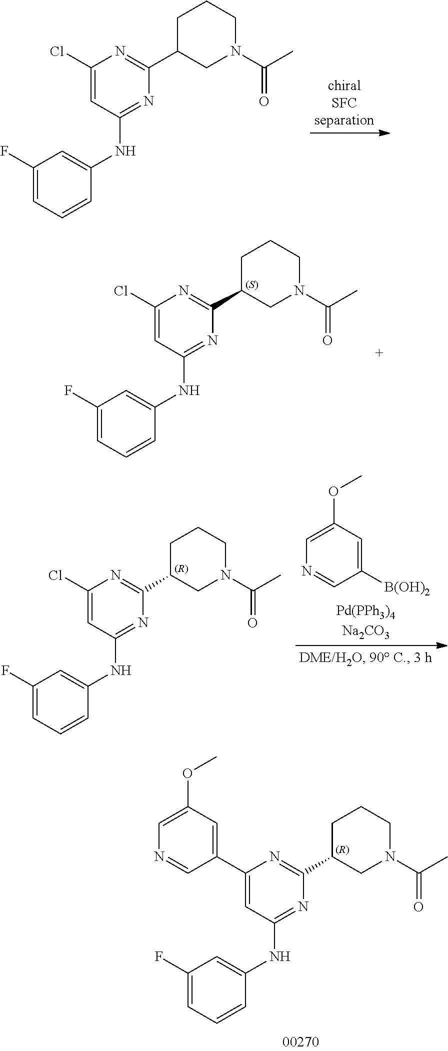

A racemic mixture of 1-(3-(4-chloro-6-((3-fluorophenyl)amino)pyrimidin-2-yl)piperidin-1-yl)ethan-1-one (200 mg) was separated using chiral preparative SFC (Column: SFC instrument modules: Waters Prep100q SFC System, PDA: Waters 2998, Fraction Collector: Waters 2767; Column: Phenomenex Lux Amylose-1 (250×20 mm, 5 μm), column temp: 35° C.; flow: 100 ml/min; ABPR: 170 bar; Eluent A: CO₂, Eluent B: 20 mM ammonia in methanol; isocratic 10% B, time: 30 min, detection: PDA (210-320 nm); fraction collection based on PDA) to afford (S)-1-(3-(4-chloro-6-((3-fluorophenyl)amino)pyrimidin-2-yl)piperidin-1-yl)ethan-1-one (70 mg, 35%): specific optical rotation [α]_{D}^{24.1}: 94.69 (c=0.11, methanol); Chiral UPLC (Method: SFC instrument modules: Waters Prep100q SFC System, PDA: Waters 2998; Column: Phenomenex Amylose-1 (100×4.6 mm, 5 μm), column temp: 35° C.; flow: 2.5 ml/min; ABPR: 170 bar; Eluent A: CO₂, Eluent B: methanol with 20 mM ammonia; t=0 min 0% B, t=5 min 5% B, t=6 min 50% B, detection: PDA (210-320 nm); fraction collection based on PDA) t_{R} 3.13 min, >95% ee and (R)-1-(3-(4-chloro-6-((3-fluorophenyl)amino)pyrimidin-2-yl)piperidin-1-yl)ethan-1-one (67 mg, 33%): specific optical rotation [α]_{D}^{24.3}: −94.56 (c=0.12, methanol); Chiral UPLC (Method: SFC instrument modules: Waters Prep100q SFC System, PDA: Waters 2998; Column: Phenomenex Amylose-1 (100×4.6 mm, 5 μm), column temp: 35° C.; flow: 2.5 ml/min; ABPR: 170 bar; Eluent A: CO₂, Eluent B: methanol with 20 mM ammonia; t=0 min 10% B, t=8 min 40% B, t=9 min 40% B, detection: PDA (210-320 nm); fraction collection based on PDA) t_{R} 3.84 min, >95% ee. Under argon atmosphere, (R)-1-(3-(4-chloro-6-((3-fluorophenyl)amino)pyrimidin-2-yl)piperidin-1-yl)ethan-1-one (10 mg, 0.03 mmol), sodium carbonate (12.2 mg, 0.12 mmol) and 5-methoxypyridine-3-boronic acid (8.8 mg, 0.06 mmol) were dissolved in 1,2-dimethoxyethane (1 mL) and water (0.3 mL). Next, the mixture was heated to 60° C. and tetrakis(triphenylphosphine)palladium(0) (1.2 mg, 1.04 μmol) was added. The mixture was stirred at 90° C. for 3 hours and was concentrated in vacuo. The residue was purified by reverse phase chromatography (Method B) to afford (R)-1-(3-(4-((3-fluorophenyl)amino)-6-(5-methoxypyridin-3-yl)pyrimidin-2-yl)piperidin-1-yl)ethan-1-one (6 mg, 50%) as a white solid. ¹H-NMR (400 MHz, DMSO-d6) mixture of rotamers δ 10.10-9.88 (m, 1H), 8.89-8.74 (m, 1H), 8.43 (t, J=2.4 Hz, 1H), 7.98-7.80 (m, 2H), 7.48-7.32 (m, 2H), 7.19 (d, J=5.3 Hz, 1H), 6.89-6.79 (m, 1H), 4.80-4.64 (m, 0.5H), 4.27-4.09 (m, 1H), 3.93 (s, 3H), 3.89-3.78 (m, 0.5H), 3.52 (dd, J=13.5, 10.1 Hz, 0.5H), 3.16-3.04 (m, 0.5H), 3.02-2.87 (m, 1H), 2.86-2.74 (m, 1H), 2.30-2.18 (m, 1H), 2.04 (s, 3H), 1.96-1.71 (m, 2H), 1.68-1.41 (m, 1H); LCMS (Method): t_{R} 3.11 min, 99%, MS (ESI) 422.2 (M+H)⁺.

### Example 33: Synthesis of N-(2-(2-(2-((5-(2-(1-acetylpiperidin-3-yl)-6-((3-fluorophenyl)amino)pyrimidin-4-yl)pyridin-3-yl)oxy)ethoxy)ethoxy)ethyl)acetamide (00271)

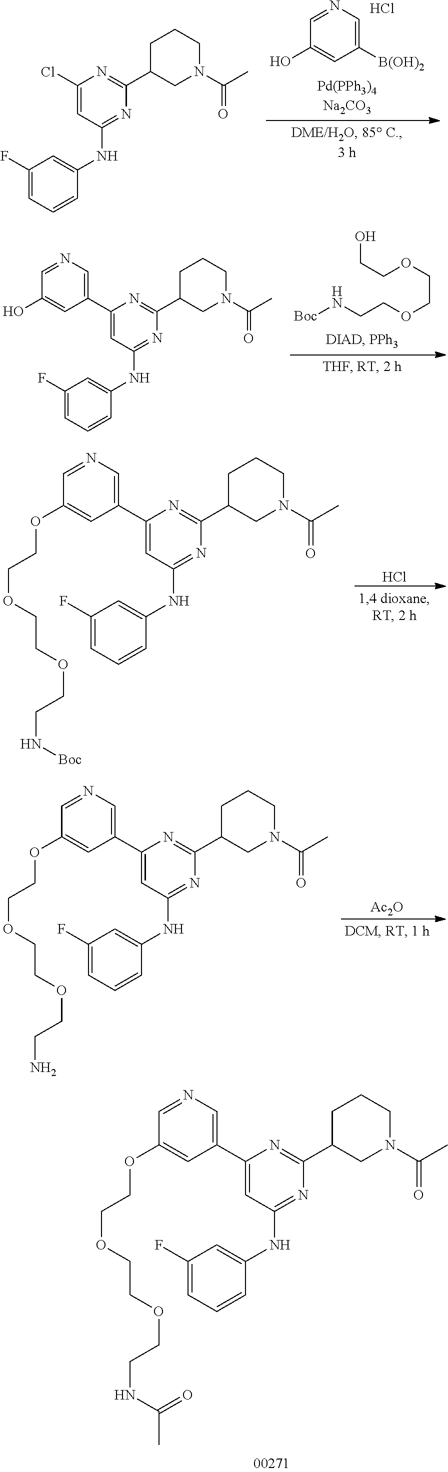

Under argon atmosphere, a microwave vial was charged with 1-(3-(4-chloro-6-((3-fluorophenyl)amino)pyrimidin-2-yl)piperidin-1-yl)ethan-1-one (300 mg, 0.86 mmol), (5-hydroxypyridin-3-yl)boronic acid hydrochloride (250 mg, 1.43 mmol), tetrakis(triphenylphosphine)palladium (99 mg, 0.09 mmol) and sodium carbonate (365 mg, 3.44 mmol) in 1,2-dimethoxyethane (16 mL) and water (4 mL). The resulting mixture was heated in a microwave at 85° C. for 3 hours, poured into water and extracted with ethyl acetate twice. The combined organic layers were washed with brine, dried with sodium sulfate and concentrated in vacuo to afford a brown solid that was purified with silica column chromatography (0% to 10% methanol in dichloromethane) to afford 1-(3-(4-((3-fluorophenyl)amino)-6-(5-hydroxypyridin-3-yl)pyrimidin-2-yl)piperidin-1-yl)ethan-1-one (110 mg, 31%) as a white solid. LCMS (Method C): t_{R} 1.77 min, 100%, MS (ESI) 408.2 (M+H)⁺. To a solution of triphenylphosphine (48.3 mg, 0.18 mmol), tert-butyl (2-(2-(2-hydroxyethoxy)ethoxy)ethyl)carbamate (33.7 mg, 0.14 mmol) and 1-(3-(4-((3-fluorophenyl)amino)-6-(5-hydroxypyridin-3-yl)pyrimidin-2-yl)piperidin-1-yl)ethan-1-one (50 mg, 0.12 mmol) in tetrahydrofuran (3 mL) was added diisopropyl azodicarboxylate (0.04 mL, 0.184 mmol) and the mixture was stirred at room temperature for 2 hours. The mixture was concentrated in vacuo to afford crude tert-butyl (2-(2-(2-((5-(2-(1-acetylpiperidin-3-yl)-6-((3-fluorophenyl)amino)pyrimidin-4-yl)pyridin-3-yl)oxy)ethoxy)ethoxy)ethyl)carbamate as a yellow gum. The crude was dissolved in 1,4-dioxane (2 ml), 4M hydrochloric acid in 1,4-dioxane (2 mL, 8 mmol) was added and the mixture was stirred at room temperature for 2 hours. The mixture was concentrated and purified with SCX (ion exchange) chromatography (washed with methanol and eluted with 3.5M ammonia in methanol) to afford 1-(3-(4-(5-(2-(2-(2-aminoethoxy)ethoxy)ethoxy)pyridin-3-yl)-6-((3-fluorophenyl)amino)pyrimidin-2-yl)piperidin-1-yl)ethan-1-one (56 mg, 85% over two steps) as a colorless gum. LCMS (Method C): t_{R} 1.89 min, 69%, MS (ESI) 539.2 (M+H)⁺. To a solution of 1-(3-(4-(5-(2-(2-(2-aminoethoxy)ethoxy)ethoxy)pyridin-3-yl)-6-((3-fluorophenyl)amino)pyrimidin-2-yl)piperidin-1-yl)ethan-1-one (56 mg, 0.104 mmol) in dichloromethane (2 mL) was added acetic anhydride (0.1 mL, 1.06 mmol) and the mixture was stirred at room temperature for 1 hour. The mixture was concentrated and purified by reversed phase chromatography (Method B) to afford N-(2-(2-(2-((5-(2-(1-acetylpiperidin-3-yl)-6-((3-fluorophenyl)amino)pyrimidin-4-yl)pyridin-3-yl)oxy)ethoxy)ethoxy)ethyl)acetamide (23 mg, 38%) as a white solid. ¹H-NMR (400 MHz, DMSO-d6) δ 10.00 (d, J=12.5 Hz, 1H), 8.80 (d, J=5.7 Hz, 1H), 8.44 (d, J=2.4 Hz, 1H), 7.97-7.83 (m, 3H), 7.47-7.33 (m, 2H), 7.20 (d, J=5.5 Hz, 1H), 6.84 (t, J=8.3 Hz, 1H), 4.71 (d, J=12.5 Hz, 0.5H), 4.29 (t, J=4.5 Hz, 2H), 4.26-4.09 (m, 1H), 3.89-3.77 (m, 2.5H), 3.65-3.48 (m, 5H), 3.40 (t, J=5.9 Hz, 2H), 3.23-3.14 (m, 2H), 3.10 (t, J=12.5 Hz, 0.5H), 3.01-2.88 (m, 1H), 2.84-2.74 (m, 1H), 2.28-2.17 (m, 1H), 2.04 (s, 3H), 1.95-1.72 (m, 5H), 1.65-1.42 (m, 1H); LCMS (Method D): t_{R} 3.05 min, 92%, MS (ESI) 581.3 (M+H)⁺.

### Example 34: Synthesis of (+/−)-cis-1-(5-(4-((3-fluorophenyl)amino)-6-(5-hydroxypyridin-3-yl)pyrimidin-2-yl)-2-methylpiperidin-1-yl)ethan-1-one (MCT00272)

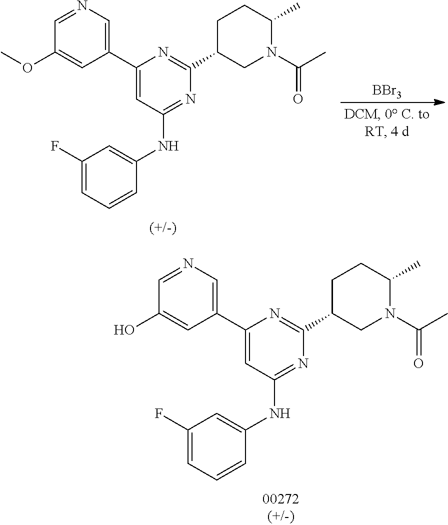

To a suspension of 1-(5-(4-((3-fluorophenyl)amino)-6-(5-methoxypyridin-3-yl)pyrimidin-2-yl)-2-methylpiperidin-1-yl)ethan-1-one (50 mg, 0.115 mmol) in dichloromethane (2 mL) at 0° C. was slowly added boron tribromide (1 M in dichloromethane, 0.92 mL, 0.92 mmol). The reaction mixture was allowed to warm up to room temperature and was stirred for 1 day. The reaction mixture was cooled to 0° C., additional boron tribromide (1M in dichloromethane, 0.92 mL, 0.92 mmol). The reaction mixture was allowed to warm up to room temperature and was stirred for 1 additional day. The reaction mixture was cooled to 0° C., additional boron tribromide (1M in dichloromethane, 2.4 mL, 2.4 mmol). The reaction mixture was allowed to warm up to room temperature and was stirred for 5 additional days. The reaction mixture was slowly added to an ice-cooled solution of aqueous sodium carbonate. The layers were separated and the aqueous layer was extracted twice with ethyl acetate. The combined organic layers were dried over sodium sulfate, concentrated and purified by reversed phase chromatography (Method A) to afford (+/−)-cis-1-(5-(4-((3-fluorophenyl)amino)-6-(5-hydroxypyridin-3-yl)pyrimidin-2-yl)-2-methylpiperidin-1-yl)ethan-1-one (21 mg, 43%) as a white solid. ¹H-NMR (400 MHz, DMSO-d6) mixture of rotamers δ 10.26 (s, 1H), 9.97 (d, J=8.3 Hz, 1H), 8.66 (d, J=6.2 Hz, 1H), 8.25 (s, 1H), 7.96 (t, J=12.8 Hz, 1H), 7.80 (q, J=2.6 Hz, 1H), 7.42-7.34 (m, 2H), 7.13 (d, J=3.0 Hz, 1H)), 6.88-6.80 (d, 1H), 4.90-4.78 (n, 0.5H), 4.72 (dd, J=13.2, 4.3 Hz, 0.5H), 4.27-4.15 (t, 1H), 4.09-4.00 (m, 0.5H), 3.49-3.43 (m, 0.5H), 2.94-2.84 (m, 1H), 2.77-2.69 (m, 0.5H), 2.10-1.94 (m, 5H), 1.90-1.79 (m, 0.5H), 1.76-1.63 (m, 1.5H), 1.28 (d, J4=6.9 Hz, 1.5H), 1.15 (d, J=7.0 Hz, 1.5H); LCMS (Method B): t_{R} 2.89 min, 99%, MS (ESI) 422.1 (M+H)⁺.

The following compounds were prepared using procedures analogous to Example 34.

| Compound # | Structure and compound name | Analytical data |
|---|---|---|
| 00273 | 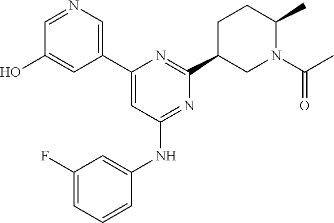 [SMILES: CC(=O)N1C[C@@H](c2nc(Nc3cccc(F)c3)cc(-c3cncc(O)c3)n2)CC[C@H]1C] (+)-1-(5-(4-((3-fluorophenyl)amino)- 6-(5-hydroxypyridin-3-yl)pyrimidin- 2-yl)-2-methylpiperidin-1-yl)ethan-1-one | ¹H NMR (400 MHz, DMSO-d6) mixture of rotamers δ 10.29 (s, 1H), 9.97 (d, J = 8.0 Hz, 1H), 8.65 (dd, J = 5.6, 1.8 Hz, 1H), 8.24 (d, J = 2.6 Hz, 1H), 7.97 (t, J = 12.6 Hz, 1H), 7.79 (q, J = 2.4 Hz, 1H), 7.42 − 7.33 (m, 2H), 7.13 (d, J = 2.9 Hz, 1H), 6.88- 6.80 (m, 1H), 4.88 − 4.79 (m, 0.5H), 4.72 (dd, J = 13.1, 4.2 Hz, 0.5H), 4.27 − 4.17 (m, 0.5H), 4.05 (dd, J = 13.7, 4.3 Hz, 0.5H), 3.44 (dd, J = 13.7, 11.8 Hz, 0.5H), 2.89 (t, J = 12.6 Hz, 1H), 2.78 − 2.70 (m, 0.5H), 2.11 − 1.97 (m, 5H), 1.89 − 1.77 (m, 0.5H), 1.77 − 1.61 (m, 1.5H), 1.27 (d, J = 6.8 Hz, 1.5H), 1.15 (d, J = 7.0 Hz, 1.5H); LCMS (Method D): t_{R} 3.00 min, 99%, MS (ESI) 422.1 (M + H)⁺, specific optical rotation |
| | | [α]_{D}^{24.4}: 33.6 (c = 0.05, methanol) |
| 00274 | 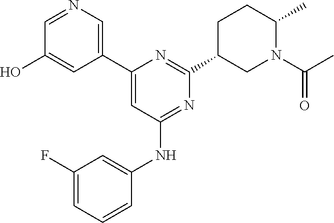 [SMILES: CC(=O)N1C[C@H](c2nc(Nc3cccc(F)c3)cc(-c3cncc(O)c3)n2)CC[C@@H]1C] (−)-1-(5-(4-((3-fluorophenyl)amino)- 6-(5-hydroxypyridin-3-yl)pyrimidin- 2-yl)-2-methylpiperidin-1-yl)ethan-1-one | ¹H NMR (400 MHz, DMSO-d6) mixture of rotamers δ 10.29 (s, 1H), 9.97 (d, J = 8.0 Hz, 1H), 8.65 (dd, J = 5.6, 1.8 Hz, 1H), 8.24 (d, J = 2.6 Hz, 1H), 7.97 (t, J = 12.6 Hz, 1H), 7.79 (q, J = 2.4 Hz, 1H), 7.42 − 7.33 (m, 2H), 7.13 (d, J = 2.9 Hz, 1H), 6.88 − 6.80 (m, 1H), 4.88 − 4.79 (m, 0.5H), 4.72 (dd, J = 13.1, 4.2 Hz, 0.5H), 4.27 − 4.17 (m, 0.5H), 4.05 (dd, J = 13.7, 4.3 Hz, 0.5H), 3.44 (dd, J = 13.7, 11.8 Hz, 0.5H), 2.89 (t, J = 12.6 Hz, 1H), 2.78 − 2.70 (m, 0.5H), 2.11 − 1.97 (m, 5H), 1.89 − 1.77 (m, 0.5H), 1.77 − 1.61 (m, 1.5H), 1.27 (d, J = 6.8 Hz, 1.5H), 1.15 (d, J = 7.0 Hz, 1.5H); LCMS (Method D): t_{R} 2.99 min, 100%, MS (ESI) 422.1 (M + H)⁺ [α]_{D}^{24.4}: −29.0 (c = 0.23, methanol) |

### Example 35: Synthesis of 1-(3-(4-((3-fluorophenyl)amino)-6-(1H-1,2,3-triazol-5-yl)pyrimidin-2-yl)piperidin-1-yl)ethan-1-one (00276)

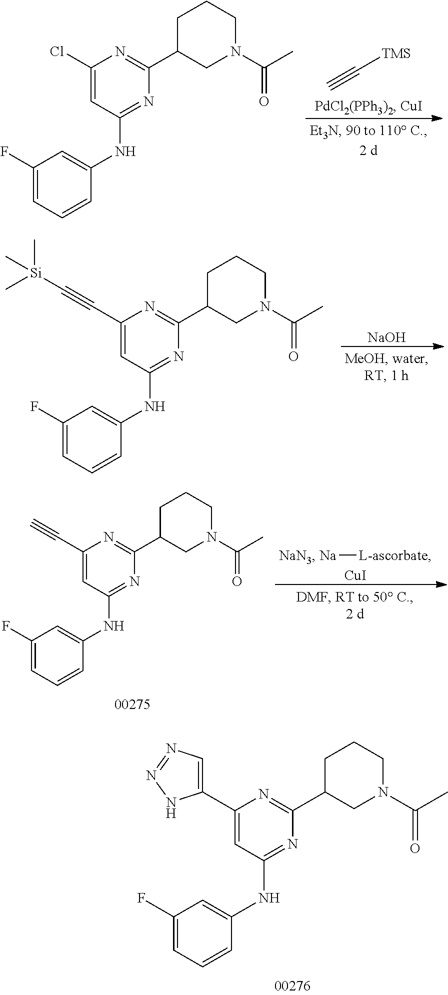

To a mixture of 1-(3-(4-chloro-6-((3-fluorophenyl)amino)pyrimidin-2-yl)piperidin-1-yl)ethan-1-one (0.20 g, 0.57 mmol), bis(triphenylphosphine)palladium(II) chloride (20 mg, 30 μmol) and copper(I) iodide (11 mg, 57 μmol) in triethylamine (1 mL) was added ethynyltrimethylsilane (0.30 mL, 2.2 mmol) and the reaction mixture was stirred at 90° C. for 1 day. A second portion of bis(triphenylphosphine)palladium(II) chloride (20 mg, 30 μmol), copper(I) iodide (11 mg, 57 μmol) and ethynyltrimethylsilane (0.30 mL, 2.2 mmol) was added and the reaction mixture was stirred at 110° C. for 1 day. The reaction mixture was diluted with ethyl acetate (100 mL) and washed with saturated aqueous ammonium chloride (100 mL). The layers were separated and the water layer was extracted with ethyl acetate (50 mL). The combined organic layers were washed with brine (50 mL), dried over sodium sulfate and purified with flash column chromatography (0% to 70% ethyl acetate in n-heptane) to afford 1-(3-(4-((3-fluorophenyl)amino)-6-((trimethylsilyl)ethynyl)pyrimidin-2-yl)piperidin-1-yl)ethan-1-one (148 mg, 63%) as an off-white solid. ¹H NMR (400 MHz, Chloroform-d) mixture of rotamers δ 7.38-7.28 (m, 2H), 7.09 (dd, J=8.2, 2.6 Hz, 1H), 6.92-6.78 (m, 2H), 6.69 (d, J=6.1 Hz, 1H), 4.92-4.82 (m, 0.4H), 4.51-4.43 (m, 0.6H), 4.03-3.96 (m, 0.6H), 3.89-3.78 (m, 0.4H), 3.50 (dd, J=13.3, 10.6 Hz, 0.6H), 3.15-3.03 (m, 0.4H), 2.97-2.84 (m, 1.4H), 2.82-2.72 (m, 0.6H), 2.52-2.12 (m, 4H), 1.93-1.73 (m, 2H), 1.52 (s, 1H), 0.26 (s, 9H); LCMS (Method A): t_{R} 2.15 min, 98%, MS (ESI) 411.2 (M+H)⁺. To a solution of 1-(3-(4-((3-fluorophenyl)amino)-6-((trimethylsilyl)ethynyl)pyrimidin-2-yl)piperidin-1-yl)ethan-1-one (140 mg, 0.34 mmol) in methanol (5 mL) was added sodium hydroxide (0.20 g, 5.0 mmol) and water (1 mL). The, reaction mixture was stirred at room temperature for 1 day, poured into saturated aqueous ammonium chloride (50 mL) and extracted with ethyl acetate (2×50 mL). The combined organic layers were washed with brine (50 mL), dried over sodium sulfate and purified using flash column chromatography (20% to 100% ethyl acetate in n-heptane) to obtain 1-(3-(4-ethynyl-6-((3-fluorophenyl)amino)pyrimidin-2-yl)piperidin-1-yl)ethan-1-one (100 mg, 87%) as a white solid. ¹H NMR (400 MHz, Chloroform-d) mixture of rotamers δ 7.40-7.27 (m, 2H), 7.16-7.04 (m, 2H), 6.92-6.80 (m, 1H), 6.76-6.67 (m, 1H), 4.91-4.84 (m, 0.4H), 4.51-4.41 (m, 0.6H), 4.06-3.97 (m, 0.6H), 3.91-3.77 (m, 0.4H), 3.52 (dd, J=13.4, 10.5 Hz, 0.6H), 3.23 (s, 0.6H), 3.20 (s, 0.4H), 3.15-3.06 (m, 0.4H), 2.98-2.74 (m, 2H), 2.28-2.17 (m, 1H), 2.15 (d, J=4.4 Hz, 3H), 1.96-1.74 (m, 2H), 1.65-1.49 (m, 1H); LCMS (Method B): t_{R} 3.05 min, 99%, MS (ESI) 339.1 (M+H)⁺. To a solution of 1-(3-(4-ethynyl-6-((3-fluorophenyl)amino)pyrimidin-2-yl)piperidin-1-yl)ethan-1-one (30 mg, 89 μmol) in dry N,N-Dimethylformamide (1 mL) was added sodium azide (5.8 mg, 89 μmol) and sodium (R)-5-((S)-1,2-dihydroxyethyl)-4-hydroxy-2-oxo-2,5-dihydrofuran-3-olate (18 mg, 89 μmol). To this suspension was added copper(I) iodide (1.7 mg, 8.9 μmol) and the reaction mixture was stirred at room temperature overnight. The reaction mixture was heated to 50° C. and stirred for 1 day, poured into saturated aqueous sodium bicarbonate (25 mL) and extracted with ethyl acetate (2×25 mL). The combined organic layers were washed with brine, dried over sodium sulfate and concentrated to obtain the crude product as a yellow oil. This was purified by reversed phase chromatography (Method B) to afford 1-(3-(4-((3-fluorophenyl)amino)-6-(1H-1,2,3-triazol-5-yl)pyrimidin-2-yl)piperidin-1-yl)ethan-1-one (6 mg, 18%) as a white solid. ¹H NMR (400 MHz, DMSO-d₆) mixture of rotamers δ 9.95 (d, J=9.7 Hz, 1H), 8.45 (s, 1H), 7.94-7.80 (m, 1H), 7.46-7.22 (m, 3H), 6.91-6.76 (m, 1H), 4.79-4.63 (m, 0.5H), 4.26-4.16 (m, 0.5H), 4.16-4.06 (m, 0.5H), 3.91-3.80 (m, 0.5H), 3.51 (dd, J=13.5, 10.1 Hz, 0.5H), 3.17-3.01 (m, 0.5H), 2.97-2.70 (m, 2H), 2.20 (d, J=12.2 Hz, 1H), 2.04 (d, J=2.2 Hz, 3H), 1.93-1.70 (m, 2H), 1.64-1.37 (m, 1H); LCMS (Method B): t_{R} 2.87 min, 98%, MS (ESI) 382.1 (M+H)⁺.

### Example 36: Synthesis of 1-(3-(4-((3-fluorophenyl)amino)-6-(pyridin-3-yl)pyrimidin-2-yl)-5-hydroxypiperidin-1-yl)ethan-1-one (00277)

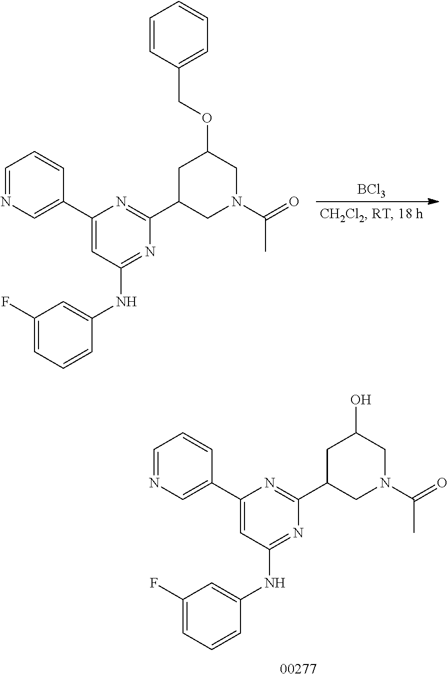 [SMILES: CC(=O)N1CC(OCc2ccccc2)CC(c2nc(Nc3cccc(F)c3)cc(-c3cccnc3)n2)C1 ; "BCl3" ; CC(=O)N1CC(O)CC(c2nc(Nc3cccc(F)c3)cc(-c3cccnc3)n2)C1]

To a solution of 1-(3-(benzyloxy)-5-(4-((3-fluorophenyl)amino)-6-(pyridin-3-yl)pyrimidin-2-yl)piperidin-1-yl)ethan-1-one (265 mg, 0.533 mmol) in dichloromethane (5 mL) in the presence of 4 Å molecular sieves was added boron trichloride (1M) in dichloromethane (1M, 3.2 mL, 3.2 mmol) at −78° C. and the mixture was allowed to come to room temperature overnight. The reaction mixture was diluted with ammonia in methanol (7N, 5 mL), stirred for 2 hours, concentrated in vacuo and resuspended in water and ethyl acetate. The layers were separated and the aqueous phase was extracted with ethyl acetate. The combined organic layers were dried over sodium sulfate, filtered and concentrated. The residue was purified by reversed phase chromatography (Method B) to afford 1-(3-(4-((3-fluorophenyl)amino)-6-(pyridin-3-yl)pyrimidin-2-yl)-5-hydroxypiperidin-1-yl)ethan-1-one (18.9 mg, 8%) as a yellow fluffy solid. ¹H NMR (400 MHz, DMSO-d₆) mixture of diastereoisomers and rotamers δ 10.05-9.92 (m, 1H), 9.24-9.16 (m, 1H), 8.75-8.66 (m, 1H), 8.43-8.32 (m, 1H), 7.96-7.74 (m, 1H), 7.64-7.53 (m, 1H), 7.47-7.33 (m, 2H), 7.22-7.13 (m, 1H), 6.92-6.80 (m, 1H), 5.16 (dd, J=15.8, 4.7 Hz, 0.5H), 5.04-4.92 (m, 0.3H), 4.83 (d, J=4.2 Hz, 0.12H), 4.81-4.67 (m, 0.5H), 4.61-4.52 (m, 0.3H), 4.42-4.39 (m, 0.04H), 4.25-4.17 (m, 0.3H), 4.02-3.86 (m, 0.8H), 3.79-3.60 (m, 0.8H), 3.56-3.45 (m, 0.3H), 3.30-3.14 (m, 1H), 3.06-2.96 (m, 0.4H), 2.93-2.76 (m, 0.9H), 2.73-2.67 (m, 0.2H), 2.30-2.22 (m, 0.3H), 2.20-2.13 (m, 0.5H), 2.11-1.92 (m, 3.3H), 1.77-1.57 (m, 0.7H); LCMS (Method D): t_{R} 2.80 min, 100%, MS (ESI) 408.1 (M+H)⁺.

### Example 37: Synthesis of 3-(2-(1-acetylpiperidin-3-yl)-6-((3-fluorophenyl)amino)pyrimidin-4-yl)pyridine 1-oxide (00278)

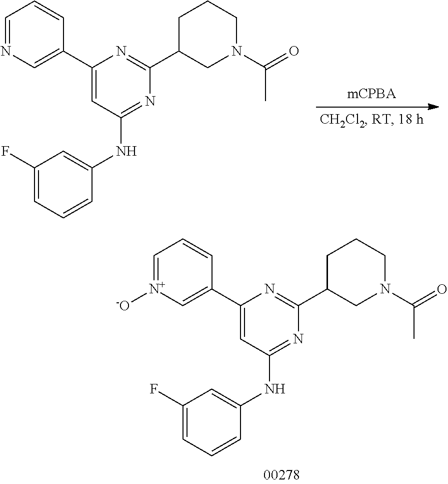

To a solution of 1-(3-(4-((3-fluorophenyl)amino)-6-(pyridin-3-yl)pyrimidin-2-yl)piperidin-1-yl)ethan-1-one (10 mg, 0.026 mmol) in dichloromethane (2 mL) mCPBA (6.30 mg, 0.026 mmol, 70%) was added and the mixture was stirred at room temperature for 18 hours. The mixture was concentrated, taken up in methanol and the residue was purified by reversed phase chromatography (Method B) to afford 3-(2-(1-acetylpiperidin-3-yl)-6-((3-fluorophenyl)amino)pyrimidin-4-yl)pyridine 1-oxide (5.5 mg, 53%) as a white fluffy solid. ¹H NMR (400 MHz, DMSO-d₆) mixture of rotamers δ 10.06 (d, J=11.4 Hz, 1H), 8.86-8.71 (m, 1H), 8.35 (dd, J=5.1, 3.2 Hz, 1H), 7.97-7.79 (m, 2H), 7.62-7.55 (m, 1H), 7.46-7.34 (m, 2H), 7.16 (d, J=5.1 Hz, 1H), 6.90-6.82 (m, 1H), 4.79-4.67 (m, 0.5H), 4.30-4.20 (m, 0.5H), 4.18-4.10 (m, 0.5H), 3.91-3.80 (m, 0.5H), 3.53-3.42 (m, 0.5H), 3.15-3.03 (m, 0.6H), 3.01-2.64 (m, 2.4H), 2.27-2.17 (m, 1H), 1.91-1.71 (m, 2H), 1.65-1.40 (m, 1H); LCMS (Method D): t_{R} 2.88 min, 99%, MS (ESI) 408.1 (M+H)⁺.

### Example 38: Synthesis of 1-(3-(4-((3-fluorophenyl)amino)-6-(5-(oxetan-3-yl)pyridin-3-yl)pyrimidin-2-yl)piperidin-1-yl)ethan-1-one (00279)

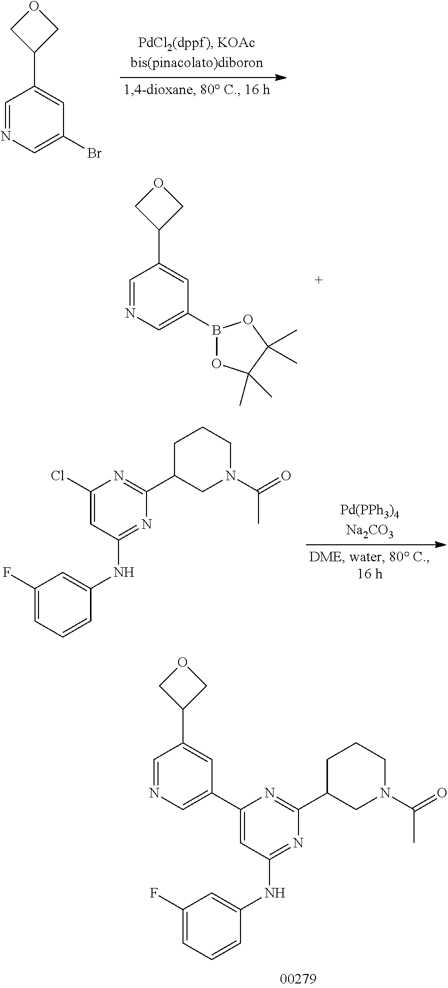

To a solution of 3-bromo-5-(oxetan-3-yl)pyridine (90 mg, 0.42 mmol) in dry 1,4-dioxane (4 mL) under nitrogen atmosphere was added PdCl₂(dppf) (30.8 mg, 0.042 mmol), potassium acetate (124 mg, 1.26 mmol) and bis(pinacolato)diboron (128 mg, 0.51 mmol). The reaction mixture was heated to 80° C. for 16 hours, filtered over Celite and washed with ethyl acetate and water. The aqueous layer was extracted with ethyl acetate, the combined organic layers were dried over sodium sulfate and concentrated to obtain the crude product that was used as such in the next step. To a nitrogen degassed mixture of 1-(3-(4-chloro-6-((3-fluorophenyl)amino)pyrimidin-2-yl)piperidin-1-yl)ethan-1-one (40 mg, 0.115 mmol) in aqueous sodium carbonate (2 M, 0.17 mL, 0.34 mmol) and 1,2-dimethoxyethane (1 mL) was added palladiumtetrakis (6.6 mg, 5.7 μmol) and the crude product from the first step 3-(oxetan-3-yl)-5-(4,4,5,5-tetramethyl-1,3,2-dioxaborolan-2-yl)pyridine (0.42 mmol). The reaction mixture was heated to 80° C. and stirred for 16 hours. The reaction mixture was poured in saturated aqueous ammonium chloride and dichloromethane was added. The layers were separated using, the water layer was extracted with dichloromethane, the combined organic layers were concentrated and the residue was purified by reversed phase chromatography (Method A) to afford 1-(3-(4-((3-fluorophenyl)amino)-6-(5-(oxetan-3-yl)pyridin-3-yl)pyrimidin-2-yl)piperidin-1-yl)ethan-1-one (4 mg, 8%) as a white solid. ¹H NMR (400 MHz, DMSO-d₆) mixture of rotamers δ 9.99 (d, J=10.6 Hz, 1H), 9.18-9.06 (m, 1H), 8.71 (s, 1H), 8.46 (dt, J=4.2, 2.1 Hz, 1H), 7.96-7.82 (m, 1H), 7.47-7.32 (m, 2H), 7.24 (d, J=5.1 Hz, 1H), 6.85 (t, J=8.5 Hz, 1H), 5.06-4.98 (m, 2H), 4.77-4.65 (m, 2.5H), 4.48-4.36 (m, 1H), 4.25-4.09 (m, 1H), 3.85 (d, J=13.5 Hz, 0.5H), 3.53 (dd, J=13.4, 10.1 Hz, 0.5H), 3.16-3.04 (m, 0.5H), 3.04-2.74 (m, 2H), 2.24 (d, J=13.1 Hz, 1H), 2.05 (s, 3H), 1.96-1.72 (m, 2H), 1.67-1.41 (m, 1H); LCMS (Method B): t_{R} 2.83 min, 97%, MS (ESI) 448.2 (M+H)⁺.

The following compounds were prepared using procedures analogous to Example 38.

| Compound # | Structure and compound name | Analytical data |
|---|---|---|
| 00280 | 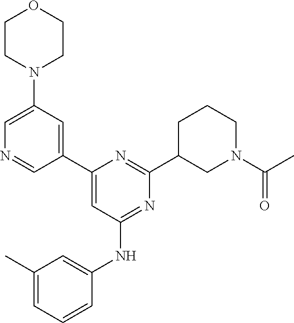 [SMILES: CC(=O)N1CCCC(c2nc(Nc3cccc(C)c3)cc(-c3cncc(N4CCOCC4)c3)n2)C1] | ¹H NMR (400 MHz, DMSO-d6) mixture of rotamers δ 9.69 − 9.57 (m, 1H), 8.64 − 8.56 (m, 1H), 8.49 − 8.38 (m, 1H), 7.87 − 7.79 (m, 1H), 7.68 − 7.59 (m, 1H), 7.58 − 7.48 (m, 1H), 7.27 − 7.18 (m, 1H), 7.15 − 7.07 (m, 1H), 6.90 − 6.80 (m, 1H), 4.81 − 4.64 (m, 0.5H), 4.22 − 4.05 (m, 1H), 3.88 − 3.81 (m, 0.5H), 3.58 − 3.49 (m, 0.5H), 3.13 − 3.03 (m, 0.5H), 2.98 − 2.70 (m, 2H), 2.35 − 2.29 (m, 3H), 2.26 − 2.14 (m, 1H), 2.09- 1.98 (m, 3H), 1.96- 1.70 (m, 2H), 1.65 − 1.38 (m, 1H); LCMS (Method D): t_{R} 3.19 min, 100%, MS (ESI) 473.2 (M + H)⁺ |
| | 1-(3-(4-(5-morpholinopyridin-3-yl)- | |
| | 6-(m-tolylamino)pyrimidin-2- | |
| | yl)piperidin-1-yl)ethan-1-one | |
| 00281 | 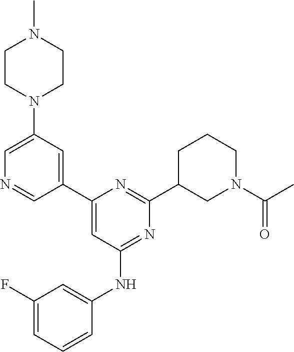 [SMILES: CC(=O)N1CCCC(c2nc(Nc3cccc(F)c3)cc(-c3cncc(N4CCN(C)CC4)c3)n2)C1] | ¹H NMR (400 MHz, Chloroform-d) mixture of rotamers δ 8.64 − 8.47 (m, 1H), 8.44 − 8.14 (m, 1H), 7.96 − 7.80 (m, 1H), 7.49 − 7.28 (m, 2H), 7.20 − 7.13 (m, 1H), 7.12 − 7.01 (m, 1H), 6.99 − 6.92 (m, 1H), 6.91 − 6.79 (m, 1H), 4.97 − 4.84 (m, 0.5H), 4.53 − 4.41 (m, 0.5H), 4.18 − 4.03 (m, 0.5H), 3.89 − 3.82 (m, 0.5H), 3.64 − 3.50 (m, 0.5H), 3.48 − 3.29 (m, 4H), 3.22 − 2.92 (m, 2H), 2.90 − 2.79 (m, 0.5H), 2.79 − 2.66 (m, 4H), 2.44 (s, 3H), 2.34 − 2.23 (m, 1H), 2.16 (s, 3H), 2.04 − 1.78 (m, 2H), 1.72 − 1.52 (m, 1H); LCMS (Method D): t_{R} 3.49 min, 99%, MS (ESI) 490.2 (M + H)⁺ |
| | 1-(3-(4-((3-fluorophenyl)amino)-6- | |
| | (5-(4-methylpiperazin-1-yl)pyridin- | |
| | 3-yl)pyrimidin-2-yl)piperidin-1- | |
| | yl)ethan-1-one | |
| 00282 | 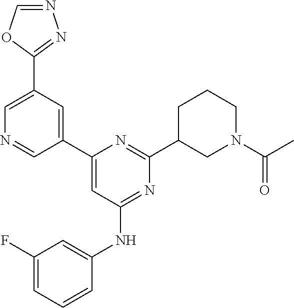 [SMILES: CC(=O)N1CCCC(c2nc(Nc3cccc(F)c3)cc(-c3cncc(-c4nnco4)c3)n2)C1] | ¹H NMR (400 MHz, DMSO-d6) mixture of rotamers δ 10.09 (s, 1H), 9.51 (d, J = 2.1 Hz, 1H), 9.46 − 9.38 (m, 1H), 9.32 (d, J = 2.0 Hz, 1H), 8.95 − 8.90 (m, 1H), 7.95 − 7.83 (m, 1H), 7.48 − 7.30 (m, 3H), 6.91 − 6.81 (m, 1H), 4.85 − 4.71 (m, 0.5H), 4.31 − 4.21 (m, 0.5H), 4.21 − 4.12 (m, 0.5H), 3.93 − 3.81 (m, 0.5H), 3.55 − 3.46 (m, 0.5H), 3.15 − 3.06 (m, 0.5H), 3.06 − 2.95 (m, 0.5H), 2.94 − 2.70 (m, 1.5H), 2.30 − 2.19 (m, 1H), 2.11 − 2.01 (m, 3H), 1.96 − 1.74 (m, 2H), 1.71 − 1.40 (m, 1H); LCMS (Method D): t_{R} 3.47 min, 100%, MS (ESI) 460.2 (M + H)⁺ |
| | 1-(3-(4-(5-(1,3,4-oxadiazol-2- | |
| | yl)pyridin-3-yl)-6-((3- | |
| | fluorophenyl)amino)pyrimidin-2- | |
| | yl)piperidin-1-yl)ethan-1-one | |
| 00283 | 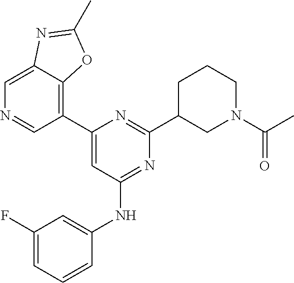 [SMILES: CC(=O)N1CCCC(c2nc(Nc3cccc(F)c3)cc(-c3cncc4nc(C)oc34)n2)C1] | ¹H NMR (400 MHz, DMSO-d6) mixture of rotamers δ 10.19 (d, J = 9.6 Hz, 1H), 9.35 (d, J = 6.9 Hz, 1H), 9.09 (d, J = 1.9 Hz, 1H), 7.92 (t, J = 12.7 Hz, 1H), 7.53 (d, J = 4.6 Hz, 1H), 7.50 − 7.45 (m, 1H), 7.44 − 7.36 (m, 1H), 6.87 (t, J = 8.3 Hz, 1H), 4.78 (d, J = 12.9 Hz, 0.5H), 4.28 − 4.15 (m, 1H), 3.86 (d, J = 13.3 Hz, 0.5H), 3.57 − 3.49 (m, 0.5H), 3.15 − 3.06 (m, 0.5H), 3.04 − 2.96 (m, 0.5H), 2.96 − 2.87 (m, 0.5H), 2.86 − 2.74 (m, 4H), 2.30 − 2.21 (m, 1H), 2.06 (d, J = 3.4 Hz, 3H), 1.96 − 1.76 (m, 2H), 1.69 − 1.42 (m, 1H); LCMS (Method D): t_{R} 3.54 min, 99%, MS (ESI) 447.1 (M + H)⁺ |
| | 1-(3-(4-((3-fluorophenyl)amino)-6- | |
| | (2-methyloxazolo[4,5-c]pyridin-7- | |
| | yl)pyrimidin-2-yl)piperidin-1- | |
| | yl)ethan-1-one | |
| 00284 | 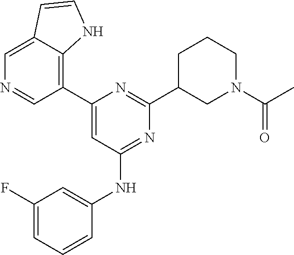 [SMILES: CC(=O)N1CCCC(c2nc(Nc3cccc(F)c3)cc(-c3cncc4cc[nH]c34)n2)C1] 1-(3-(4-((3-fluorophenyl)amino)-6- | ¹H NMR (400 MHz, DMSO-d6) mixture of rotamers δ 11.50 (s, 1H), 10.04 (d, J = 11.0 Hz, 1H), 8.94 (s, 1H), 8.71 (d, J = 8.7 Hz, 1H), 7.97 − 7.87 (m, 1H), 7.57 (s, 1H), 7.48 − 7.34 (m, 2H), 7.31 (d, J = 11.0 Hz, 1H), 6.85 (t, J = 8.4 Hz, 1H), 6.72 (s, 1H), 4.59 (d, J = 12.6 Hz, 0.5H), 4.37 (d, J = 13.0 Hz, 0.5H), 4.18 (d, J = 12.9 Hz, 0.5H), 3.83 (d, J = 13.4 Hz, 0.5H), 3.51 − 3.48 (m, 0.5H), 3.18 − 3.15 (m, 0.5H), 3.12 − 3.01 (m, 1H), 2.71 − 2.65 (m, 1H), 2.31 − 2.16 (m, 1H), 2.06 (d, J = 4.1 Hz, 3H), 1.87 (dt, J = 41.2, 13.0 Hz, 2H), 1.71 − 1.42 (m, 1H); LCMS (Method D): t_{R} 3.73 min, 99%, MS (ESI) 431.2 (M + H)⁺ |
| | (1H-pyrrolo[3,2-c]pyridin-7- | |
| | yl)pyrimidin-2-yl)piperidin-1- | |
| | yl)ethan-1-one | |
| 00285 | 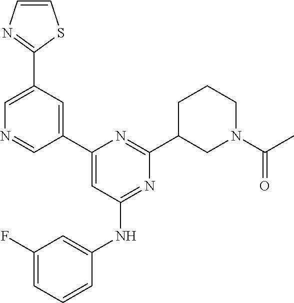 [SMILES: CC(=O)N1CCCC(c2nc(Nc3cccc(F)c3)cc(-c3cncc(-c4nccs4)c3)n2)C1] | ¹H NMR (400 MHz, DMSO-d6) mixture of rotamers δ 10.05 (d, J = 10.0 Hz, 1H), 9.35 − 9.22 (m, 2H), 8.87 (t, J = 2.2 Hz, 1H), 8.08 (d, J = 3.2 Hz, 1H), 7.97 (d, J = 3.2 Hz, 1H), 7.91 (dd, J = 13.6, 11.1 Hz, 1H), 7.48 − 7.34 (m, 2H), 7.31 (d, J = 5.3 Hz, 1H), 6.86 (t, J = 8.4 Hz, 1H), 4.77 (d, J = 12.2 Hz, 0.5H), 4.29 − 4.13 (m, 1H), 3.87 (d, J = 13.1 Hz, 0.5H), 3.53 (dd, J = 13.4, 10.2 Hz, 0.5H), 3.10 (td, J = 13.6, 12.8, 2.8 Hz, 0.5H), 3.00 (td, J = 10.4, 5.2 Hz, 0.5H), 2.95 − 2.72 (m, 1.5H), 2.26 (d, J = 13.0 Hz, 1H), 2.06 (d, J = 3.3 Hz, 3H), 1.97 − 1.72 (m, 2H), 1.57 (dt, J = 49.7, 13.0 Hz, 1H); LCMS (Method B): t_{R} 3.72 min, 99%, MS (ESI) 475.1 (M + H)⁺ |
| | 1-(3-(4-((3-fluorophenyl)amino)-6- | |
| | (5-(thiazol-2-yl)pyridin-3- | |
| | yl)pyrimidin-2-yl)piperidin-1- | |
| | yl)ethan-1-one | |
| 00286 | 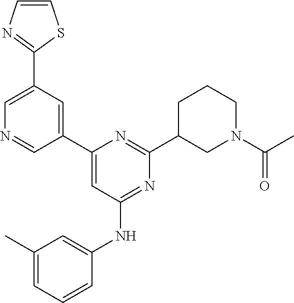 [SMILES: CC(=O)N1CCCC(c2nc(Nc3cccc(C)c3)cc(-c3cncc(-c4nccs4)c3)n2)C1] | ¹H NMR (400 MHz, DMSO-d6) mixture of rotamers δ 9.74 (d, J = 7.8 Hz, 1H), 9.31 − 9.21 (m, 2H), 8.85 (t, J = 2.2 Hz, 1H), 8.07 (d, J = 3.2 Hz, 1H), 7.97 (d, J = 3.2 Hz, 1H), 7.65 (d, J = 5.6 Hz, 1H), 7.55 (d, J = 8.1 Hz, 1H), 7.31 − 7.17 (m, 2H), 6.87 (d, J = 7.5 Hz, 1H), 4.87 − 4.73 (m, 0.5H), 4.28 − 4.07 (m, 1H), 3.93 − 3.80 (m, 0.5H), 3.54 (dd, J = 13.4, 10.1 Hz, 0.5H), 3.15 − 3.03 (m, 0.5H), 3.02 − 2.92 (m, 0.5H), 2.91 − (m, 1.5H), 2.33 (d, J = 2.7 Hz, 3H), 2.23 (d, J = 10.6 Hz, 1H), 2.05 (s, 3H), 1.98- 1.73 (m, 2H), 1.67 − 1.40 (m, 1H); LCMS (Method B): t_{R} 3.66 min, 98%, MS (ESI) 471.1 (M +H)⁺ |
| | 1-(3-(4-(5-(thiazol-2-yl)pyridin-3-yl)- | |
| | 6-(m-tolyamino)pyrimidin-2- | |
| | yl)piperidin-1-yl)ethan-1-one | |

### Example 39: Synthesis of 1-(3-(4-((3-fluorophenyl)amino)-6-(5-(morpholine-4-carbonyl)pyridin-3-yl)pyrimidin-2-yl)piperidin-1-yl)ethan-1-one (00288)

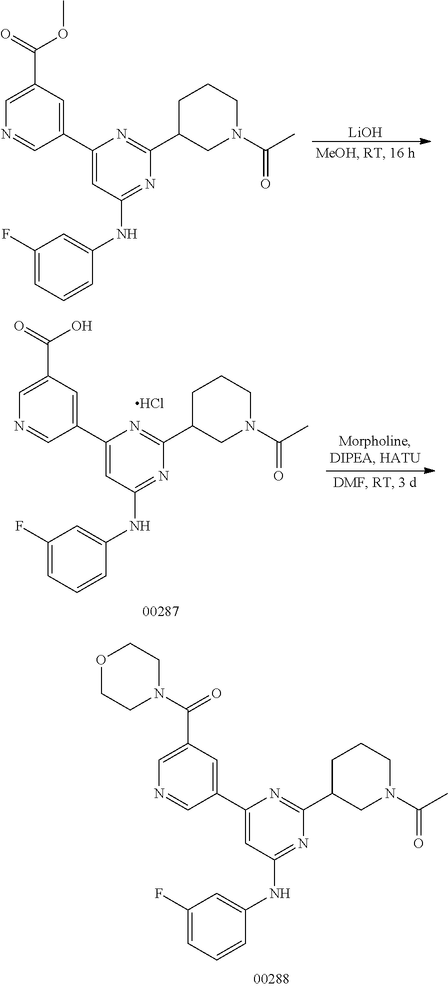

To a solution of methyl 5-(2-(1-acetylpiperidin-3-yl)-6-((3-fluorophenyl)amino)pyrimidin-4-yl)nicotinate (128 mg, 0.29 mmol) in methanol (1 mL) was added lithium hydroxide (20 mg, 0.85 mmol). The reaction mixture was stirred at room temperature for 16 hours and then acidified using aqueous hydrogen chloride solution (1M, 25 mL, 25 mmol). The resulting solids were filtered off and co-evaporated with MeOH to dryness to afford 5-(2-(1-acetylpiperidin-3-yl)-6-((3-fluorophenyl)amino)pyrimidin-4-yl)nicotinic acid hydrochloride (128 mg, 95%) as a yellow solid. ¹H NMR (400 MHz, DMSO-d₆) mixture of rotamers δ 9.98 (d, J=10.3 Hz, 1H), 9.25 (s, 1H), 9.09 (s, 1H), 8.75 (t, J=2.1 Hz, 1H), 7.90 (t, J=12.9 Hz, 1H), 7.47-7.31 (m, 2H), 7.25 (d, J=3.9 Hz, 1H), 6.84 (t, J=8.5 Hz, 1H), 4.83-4.67 (m, 0.5H), 4.31-4.11 (m, 1H), 3.91-3.82 (m, 0.5H), 3.50 (dd, J=13.4, 10.3 Hz, 0.5H), 3.14-3.04 (m, 0.5H), 3.05-2.69 (m, 2H), 2.29-2.19 (m, 1H), 2.05 (s, 3H), 1.90-1.73 (m, 2H), 1.68-1.43 (m, 1H); LCMS (Method A): t_{R} 1.86 min, 92%, MS (ESI) 436.1 (M+H)⁺. To a solution of 5-(2-(1-acetylpiperidin-3-yl)-6-((3-fluorophenyl)amino)pyrimidin-4-yl)nicotinic acid hydrochloride (30 mg, 70 μmol) in dry N,N-dimethylformamide (1 mL) was added 1-[bis(dimethylamino)methylene]-1H-1,2,3-triazolo[4,5-b]pyridinium 3-oxid hexafluorophosphate (29 mg, 76 μmol), N,N-diisopropylethylamine (36 μL, 0.21 mmol) and morpholine (7.0 μL, 83 μmol) and the reaction mixture was stirred at room temperature for three days. The reaction mixture was directly purified by reversed phase chromatography (Method A) to afford 1-(3-(4-((3-fluorophenyl)amino)-6-(5-(morpholine-4-carbonyl)pyridin-3-yl)pyrimidin-2-yl)piperidin-1-yl)ethan-1-one (13 mg, 37%) as a light yellow solid. ¹H NMR (400 MHz, DMSO-d₆) mixture of rotamers δ 10.02 (s, 1H), 9.26 (dd, J=6.0, 2.1 Hz, 1H), 8.76 (t, J=1.8 Hz, 1H), 8.41 (q, J=2.1 Hz, 1H), 7.88 (tt, J=12.1, 2.3 Hz, 1H), 7.49-7.32 (m, 2H), 7.24 (d, J=4.7 Hz, 1H), 6.91-6.79 (m, 1H), 4.81-4.67 (m, 0.5H), 4.23 (d, J=13.0 Hz, 0.5H), 4.14 (dd, J=13.3, 3.9 Hz, 0.5H), 3.85 (d, J=13.4 Hz, 0.5H), 3.78-3.36 (m, 8.5H), 3.10 (td, J=13.6, 12.9, 2.8 Hz, 0.5H), 3.02-2.72 (m, 2H), 2.29-2.18 (m, 1H), 2.04 (s, 3H), 1.96-1.72 (m, 2H), 1.67-1.40 (m, 1H); LCMS (Method D): t_{R} 2.96 min, 98%, MS (ESI) 505.2 (M+H)⁺.

The following compounds were prepared using procedures analogous to Example 39.

| Compound # | Structure and compound name | Analytical data |
|---|---|---|
| 00289 | 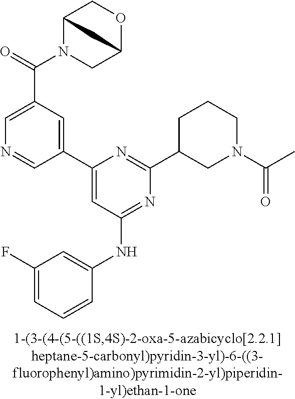 | ¹H NMR (400 MHz, DMSO-d₆) mixture of rotamers δ 10.02 (s, 1H), 9.34- 9.23 (m, 1H), 8.92-8.79 (m, 1H), 8.54-8.44 (m, 1H), 7.88 (t, J = 12.2 Hz, 1H), 7.48-7.33 (m, 2H), 7.30- 7.19 (m, 1H), 6.85 (t, J = 8.5 Hz, 1H), 4.91 (s, 0.5H), 4.72 (d, J = 15.6 Hz, 1H), 4.61 (s, 0.5H), 4.45 (s, 0.5H), 4.24 (d, J = 13.1 Hz, 0.5H), 4.19- 4.11 (m, 0.5H), 4.05-3.96 (m, 0.5H), 3.92-3.68 (m, 2H), 3.64-3.45 (m, 1.5H), 3.37 (d, J = 11.1 Hz, 1H), 3.16- 3.03 (m, 0.5H), 3.03-2.70 (m, 2H), 2.24 (d, J = 12.7 Hz, 1H), 2.04 (s, 3H), 2.01-1.71 (m, 4H), 1.67-1.39 (m, 1H); LCMS (Method D): t_{R} 2.92 min, 98%, MS (ESI) 422.1 (M + H)⁺ |
| 00290 | 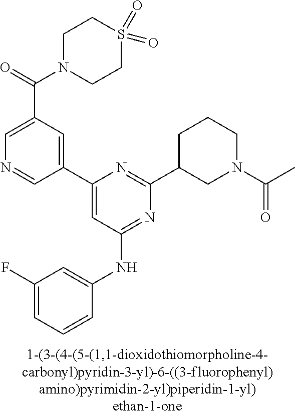 [SMILES: CC(=O)N1CCCC(c2nc(Nc3cccc(F)c3)cc(-c3cncc(C(=O)N4CCS(=O)(=O)CC4)c3)n2)C1] | ¹H NMR (400 MHz, DMSO-d₆) mixture of rotamers δ 10.02 (d, J = 8.7 Hz, 1H), 9.27 (dd, J = 6.2, 2.2 Hz, 1H), 8.83 (d, J = 2.0 Hz, 1H), 8.55-8.45 (m, 1H), 7.94-7.80 (m, 1H), 7.48- 7.33 (m, 2H), 7.22 (d, J = 4.5 Hz, 1H), 6.86 (t, J = 8.3 Hz, 1H), 4.72 (d, J = 13.2 Hz, 0.5H), 4.28-3.93 (m, 3H), 3.90-3.65 (m, 2.5H), 3.51 (dd, J = 13.5, 10.1 Hz, 0.5H), 3.31 (s, 4H), 3.10 (t, J = 12.3 Hz, 0.5H), 3.03-2.86 (m, 1H), 2.85-2.72 (m, 1H), 2.22 (s, 1H), 2.04 (s, 3H), 1.95-1.71 (m, 2H), 1.68-1.39 (m, 1H); LCMS (Method D): t_{R} 2.96 min, 99%, MS (ESI) 553.1 (M + H)⁺ |

### Example 40: Synthesis of 1-(3-(4-((3-fluorophenyl)amino)-6-(6-morpholinopyridin-3-yl)pyrimidin-2-yl)piperidin-1-yl)ethan-1-one (00291)

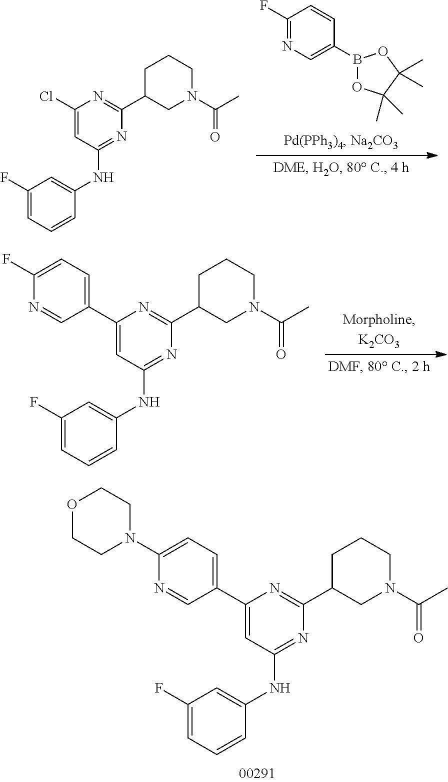

To a N₂-degassed solution of 1-(3-(4-chloro-6-((3-fluorophenyl)amino)pyrimidin-2-yl)piperidin-1-yl)ethan-1-one (50 mg, 0.14 mmol) in aqueous sodium carbonate (2M, 0.22 mL, 0.44 mmol) and 1,2-dimethoxyethane (1 mL) was added 2-fluoro-5-(4,4,5,5-tetramethyl-1,3,2-dioxaborolan-2-yl)pyridine (96 mg, 0.43 mmol) and palladium tetrakis triphenylphosphine (8.3 mg, 7.2 μmol) and the reaction mixture was stirred at 80° C. for 4 hours. The reaction mixture was directly purified by using reversed phase chromatography (Method A) to afford 1-(3-(4-((3-fluorophenyl)amino)-6-(6-fluoropyridin-3-yl)pyrimidin-2-yl)piperidin-1-yl)ethan-1-one (40 mg, 68%) as a white solid. ¹H NMR (400 MHz, DMSO-d₆) mixture of rotamers δ 10.00 (d, J=10.4 Hz, 1H), 8.89 (dd, J=6.8, 2.5 Hz, 1H), 8.67-8.51 (m, 1H), 7.97-7.79 (m, 1H), 7.48-7.30 (m, 3H), 7.15 (d, J=4.7 Hz, 1H), 6.85 (t, J=8.4 Hz, 1H), 4.73 (d, J=12.4 Hz, 0.5H), 4.23 (d, J=13.0 Hz, 0.5H), 4.14 (d, J=14.4 Hz, 0.5H), 3.85 (d, J=13.6 Hz, 0.5H), 3.49 (dd, J=13.4, 10.2 Hz, 0.5H), 3.16-3.03 (m, 0.5H), 2.93-2.84 (m, 0.5H), 2.84-2.70 (m, 0.5H), 2.29-2.17 (m, 1H), 2.04 (s, 3H), 1.95-1.72 (m, 2H), 1.69-1.39 (m, 1H); LCMS (Method D): t_{R} 3.40 min, 100%, MS (ESI) 410.1 (M+H)⁺. To a solution of 1-(3-(4-((3-fluorophenyl)amino)-6-(6-fluoropyridin-3-yl)pyrimidin-2-yl)piperidin-1-yl)ethan-1-one (40 mg, 98 μmol) in dry N,N-dimethylformamide (1 mL) was added morpholine (26 μl, 0.29 mmol) and potassium carbonate (41 mg, 0.29 mmol). The reaction mixture was stirred at 80° C. for 2 hours. A few drops of aqueous hydrogen chloride (1 M) were added to the reaction mixture to quench the potassium carbonate. The mixture was purified by using reversed phase chromatography (Method A) to afford 1-(3-(4-((3-fluorophenyl)amino)-6-(6-fluoropyridin-3-yl)pyrimidin-2-yl)piperidin-1-yl)ethan-1-one (20 mg, 43%) as a white solid. ¹H NMR (400 MHz, DMSO-d₆) mixture of rotamers δ 9.82 (d, J=9.9 Hz, 1H), 8.83 (dd, J=6.8, 2.4 Hz, 1H), 8.23-8.13 (m, 1H), 7.95-7.82 (m, 1H), 7.44-7.29 (m, 2H), 7.00 (d, J=3.7 Hz, 1H), 6.97 (d, J=9.0 Hz, 1H), 6.86-6.75 (m, 1H), 4.73 (d, J=12.5 Hz, 0.5H), 4.20 (d, J=12.9 Hz, 0.5H), 4.16-4.08 (m, 0.5H), 3.85 (d, J=13.8 Hz, 0.5H), 3.71 (dd, J=5.8, 4.0 Hz, 4H), 3.58 (t, J=4.8 Hz, 4H), 3.49 (dd, J=13.4, 10.1 Hz, 0.5H), 3.13-3.01 (m, 0.5H), 2.96-2.65 (m, 2H), 2.21 (d, J=12.3 Hz, 1H), 2.04 (s, 3H), 1.94-1.71 (m, 2H), 1.67-1.37 (m, 1H); LCMS (Method B): t_{R} 2.82 min, 98%, MS (ESI) 477.2 (M+H)⁺.

The following compounds were prepared using procedures analogous to Example 40.

| Compound # | Structure and compound name | Analytical data |
|---|---|---|
| 00292 | 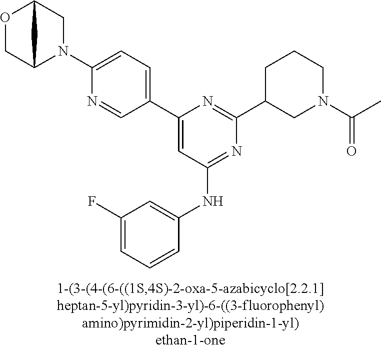 [SMILES: CC(=O)N1CCCC(c2nc(Nc3cccc(F)c3)cc(-c3ccc(N4C[C@@H]5C[C@H]4CO5)nc3)n2)C1] | ¹H NMR (400 MHz, DMSO-d6) mixture of rotamers δ 9.80 (d, J = 9.6 Hz, 1H), 8.79 (dd, J = 7.9, 2.4 Hz, 1H), 8.13 (dt, J = 8.8, 2.5 Hz, 1H), 7.95-7.82 (m, 1H), 7.44-7.28 (m, 2H), 6.98 (d, J = 3.6 Hz, 1H), 6.86-6.75 (m, 1H), 6.66 (d, J = 8.9 Hz, 1H), 4.96 (s, 1H), 4.78- 4.65 (m, 1.5H), 4.21 (d, J = 12.9 Hz, 0.5H), 4.13 (dd, J = 13.4, 3.9 Hz, 0.5H), 3.91-3.76 (m, 1.5H), 3.66 (d, J = 7.3 Hz, 1H), 3.57-3.42 (m, 1.5H), 3.30 (s, 1H), 3.14-3.01 (m, 0.5H), 2.96-2.64 (m, 2H), 2.27-2.15 (m, 1H), 2.04 (s, 3H), 1.99-1.71 (m, 4H), 1.67-1.37 (m, 1H); LCMS (Method B): t_{R} 2.61 min, 99%, MS (ESI) 489.2 (M + H)⁺ |
| 00293 | 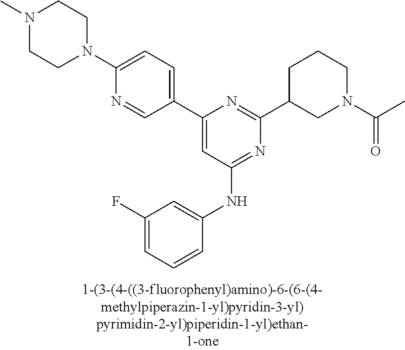 [SMILES: CC(=O)N1CCCC(c2nc(Nc3cccc(F)c3)cc(-c3ccc(N4CCN(C)CC4)nc3)n2)C1] | ¹H NMR (400 MHz, DMSO-d6) mixture of rotamers δ 9.80 (d, J = 9.8 Hz, 1H), 8.80 (dd, J = 6.9, 2.4 Hz, 1H), 8.20- 8.09 (m, 1H), 7.95-7.81 (m, 1H), 7.44-7.29 (m, 2H), 7.03-6.91 (m, 2H), 6.86-6.75 (m, 1H), 4.79-4.67 (m, 0.5H), 4.20 (d, J = 12.9 Hz, 0.5H), 4.13 (dd, J = 13.2, 4.0 Hz, 0.5H), 3.85 (d, J = 13.5 Hz, 0.5H), 3.69-3.55 (m, 4H), 3.49 (dd, J = 13.3, 10.1 Hz, 0.5H), 3.15-3.01 (m, 0.5H), 2.96- 2.69 (m, 2H), 2.40 (t, J = 5.1 Hz, 4H), 2.22 (s, 4H), 2.04 (s, 3H), 1.93-1.71 (m, 2H), 1.66-1.38 (m, 1H); LCMS (Method B): t_{R} 2.35 min, 99%, MS (ESI) 490.2 (M + H)⁺ |
| 00294 | 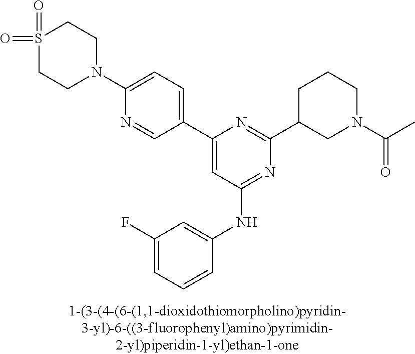 [SMILES: CC(=O)N1CCCC(c2nc(Nc3cccc(F)c3)cc(-c3ccc(N4CCS(=O)(=O)CC4)nc3)n2)C1] | ¹H NMR (400 MHz, DMSO-d₆) mixture of rotamers δ 9.83 (d, J = 10.1 Hz, 1H), 8.85 (dd, J = 6.6, 2.5 Hz, 1H), 8.21 (dt, J = 9.0, 2.5 Hz, 1H), 7.94- 7.81 (m, 1H), 7.44-7.30 (m, 2H), 7.16 (d, J = 9.0 Hz, 1H), 7.03 (d, J = 3.8 Hz, 1H), 6.86-6.77 (m, 1H), 4.73 (d, J = 12.2 Hz, 0.5H), 4.25-4.06 (m, 5H), 3.85 (d, J = 13.4 Hz, 0.5H), 3.49 (dd, J = 13.5, 10.0 Hz, 0.5H), 3.16 (t, J = 5.3 Hz, 4H), 3.08 (dd, J = 13.4, 11.1 Hz, 0.5H), 2.98-2.69 (m, 2H), 2.21 (d, J = 12.6 Hz, 1H), 2.04 (s, 3H), 1.94-1.72 (m, 2H), 1.67-1.36 (m, 1H); LCMS (Method B): t_{R} 2.88 min, 97%, MS (ESI) 525.2 (M + H)⁺ |

### Example 41: Synthesis of (+/−)-cis-1-(2-methyl-5-(4-((5-methylpyridin-3-yl)amino)-6-(pyridin-3-yl)pyrimidin-2-yl)piperidin-1-yl)ethan-1-one (00295)

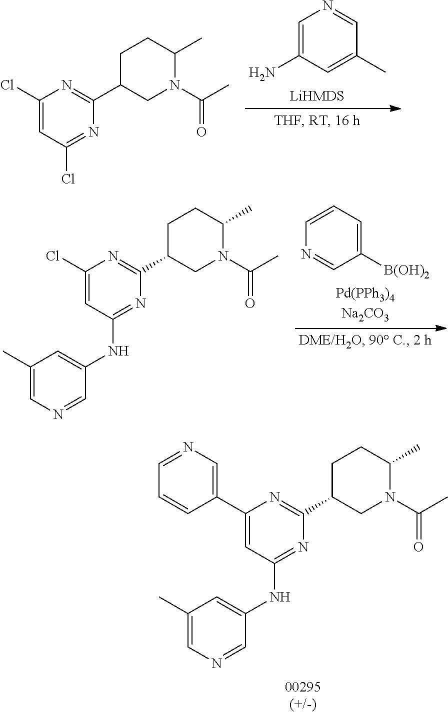

To a solution of 3-amino-5-methylpyridine (15.0 mg, 0.14 mmol) in tetrahydrofuran (2 mL) was added lithium bis(trimethylsilyl)amide in tetrahydrofuran (1M, 0.14 mL, 0.14 mmol) and the mixture was stirred at room temperature for 10 minutes. Next, 1-(5-(4,6-dichloropyrimidin-2-yl)-2-methylpiperidin-1-yl)ethan-1-one (40 mg, 0.14 mmol, ˜85:15 cis/trans mixture) in tetrahydrofuran (2 mL) was added and the mixture was stirred at room temperature for 16 hours. The mixture was poured into water and was extracted with ethyl acetate twice. The combined organic layers were washed with brine once, dried with sodium sulfate and concentrated in vacuo. The residue was purified with reverse phase chromatography (Method B) to afford (+/−)-cis-1-(5-(4-chloro-6-((5-methylpyridin-3-yl)amino)pyrimidin-2-yl)-2-methylpiperidin-1-yl)ethan-1-one (24 mg, 45%) as a white solid. ¹H NMR (400 MHz, DMSO-d6) mixture of rotamers δ 10.05 (s, 1H), 8.61 (dd, J=7.1, 2.5 Hz, 1H), 8.11 (d, J=2.6 Hz, 1H), 8.05 (d, J=8.7 Hz, 1H), 6.72 (d, J=5.9 Hz, 1H), 4.79 (s, 0.5H), 4.66 (d, J=14.0 Hz, 0.5H), 4.18 (s, 0.5H), 3.92 (dd, J=13.5, 4.3 Hz, 0.5H), 2.88-2.70 (m, 1H), 2.70-2.55 (m, 0.5H), 2.31 (d, J=3.0 Hz, 3H), 2.04 (d, J=13.4 Hz, 3H), 1.99-1.71 (m, 3H), 1.71-1.57 (m, 2H), 1.17 (dd, J=47.7, 6.9 Hz, 3H); LCMS (Method C): t_{R} 1.82 min, 100%, MS (ESI) 360.1 (M+H)⁺. Under nitrogen atmosphere, (+/−)-cis-1-(5-(4-chloro-6-((5-methylpyridin-3-yl)amino)pyrimidin-2-yl)-2-methylpiperidin-1-yl)ethan-1-one (25 mg, 0.07 mmol), sodium carbonate (14.7 mg, 0.14 mmol), pyridine-3-boronic acid (17.1 mg, 0.14 mmol) and PdCl₂(dppf) (5.7 mg, 6.9 μmol) were dissolved in 1,2-dimethoxyethane (3 mL) and water (1 mL). The mixture was heated to 80° C. for 2 hours, cooled to room temperature and eluted through a C18-plug with acetonitrile. The filtrate was purified with reverse phase chromatography (Method B) to afford (+/−)-cis-1-(2-methyl-5-(4-((5-methylpyridin-3-yl)amino)-6-(pyridin-3-yl)pyrimidin-2-yl)piperidin-1-yl)ethan-1-one (8 mg, 27%) as a beige solid. ¹H NMR (400 MHz, DMSO-d6) mixture of rotamers δ 9.93 (d, J=5.0 Hz, 1H), 9.22 (dd, J=5.8, 2.3 Hz, 1H), 8.80-8.63 (m, 2H), 8.40 (tt, J=5.5, 2.5 Hz, 1H), 8.17 (d, J=13.7 Hz, 1H), 8.09 (t, J=2.3 Hz, 1H), 7.58 (dd, J=8.0, 4.9 Hz, 1H), 7.19 (d, J=4.5 Hz, 1H), 4.83 (s, 0.5H), 4.75 (dd, J=13.0, 4.1 Hz, 0.5H), 4.27-4.18 (m, 0.5H), 4.02 (dd, J=13.7, 4.1 Hz, 0.5H), 3.44 (dd, J=13.8, 11.7 Hz, 0.5H), 2.89 (td, J=12.9, 12.2, 8.0 Hz, 1H), 2.82-2.68 (m, 0.5H), 2.33 (d, J=3.5 Hz, 3H), 2.17-1.59 (m, 7H), 1.27 (d, J=6.8 Hz, 1.5H), 1.15 (d, J=7.0 Hz, 1.5H); LCMS (Method D): t_{R} 3.14 min, 100%, MS (ESI) 403.2 (M+H)⁺.

The following compounds were prepared using procedures analogous to Example 41.

| Compound # | Structure and compound name | Analytical data |
|---|---|---|
| 00296 | 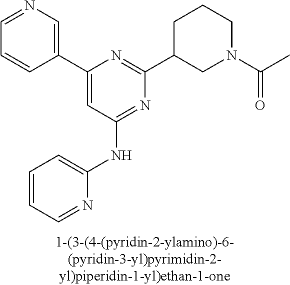 [SMILES: CC(=O)N1CCCC(c2nc(Nc3ccccn3)cc(-c3cccnc3)n2)C1] | ¹H-NMR (400 MHz, DMSO-d6) mixture of rotamers δ 10.30 (d, J = 3.6 Hz, 1H), 9.22 (t, J = 3.0 Hz, 1H), 8.81- 8.61 (m, 1H), 8.49-8.28 (m, 2H), 8.13 (s, 1H), 7.95-7.73 (m, 2H), 7.63- 7.52 (m, 1H), 7.13-6.94 (m, 1H), 4.75 (d, J = 12.4 Hz, 0.5H), 4.26 (d, J = 13.0 Hz, 0.5H), 4.12 (d, J = 14.5 Hz, 0.5H), 3.86 (d, J = 13.5 Hz, 0.5H), 3.55 (dd, J = 13.5, 10.3 Hz, 0.5H), 3.09 (t, J = 12.0 Hz, 0.5H), 3.02-2.70 (m, 2H), 2.22 (d, J = 13.0 Hz, 1H), 2.05 (d, J = 2.6 Hz, 3H), 2.00-1.71 (m, 2H), 1.66-1.40 (m, 1H); LCMS (Method D): t_{R} 3.14 min, 100%, MS (ESI) 375.2 (M + H)⁺ |
| 00297 | 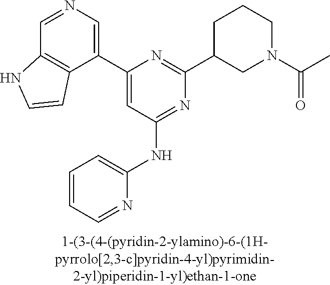 [SMILES: CC(=O)N1CCCC(c2nc(Nc3ccccn3)cc(-c3cncc4[nH]ccc34)n2)C1] | ¹H-NMR (400 MHz, DMSO-d6) mixture of rotamers δ 11.92 (s, 1H), 10.26 (d, J = 3.0 Hz, 1H), 8.86 (s, 1H), 8.78 (d, J = 3.2 Hz, 1H), 8.35 (dd, J = 4.9, 1.9 Hz, 1H), 8.26 (d, J = 5.7 Hz, 1H), 7.89 (d, J = 9.6 Hz, 1H), 7.85-7.74 (m, 2H), 7.13 (d, J = 2.9 Hz, 1H), 7.06- 6.97 (m, 1H), 4.83 (d, J = 11.2 Hz, 0.5H), 4.30 (d, J = 12.9 Hz, 0.5H), 4.21-4.13 (m, 0.5H), 3.88 (d, J = 13.4 Hz, 0.5H), 3.54 (dd, J = 13.3, 10.4 Hz, 0.5H), 3.17-3.04 (m, 0.5H), 2.99 (td, J = 10.7, 5.3 Hz, 0.5H), 2.94- 2.70 (m, 1.52H), 2.26 (d, J = 11.7 Hz, 1H), 2.06 (s, 3H), 1.99-1.73 (m, 2H), 1.68-1.42 (m, 1H); LCMS (Method D): t_{R} 3.10 min, 100%, MS (ESI) 414.2 (M + H)⁺ |
| 00298 | 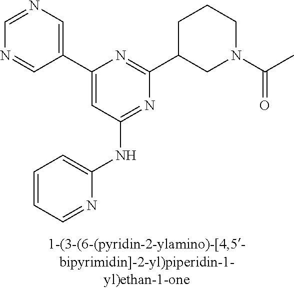 [SMILES: CC(=O)N1CCCC(c2nc(Nc3ccccn3)cc(-c3cncnc3)n2)C1] | ¹H-NMR (400 MHz, DMSO-d6) mixture of rotamers δ 10.39 (d, J = 3.2 Hz, 1H), 9.39 (d, J = 5.8 Hz, 2H), 9.34 (d, J = 2.4 Hz, 1H), 8.38 (dd, J = 4.8, 1.5 Hz, 1H), 8.20 (s, 1H), 7.92-7.75 (m, 2H), 7.11-7.01 (m, 1H), 4.73 (d, J = 12.6 Hz, 0.5H), 4.26 (d, J = 13.1 Hz, 0.5H), 4.12 (d, J = 14.2 Hz, 0.5H), 3.86 (d, J = 13.3 Hz, 0.5H), 3.56 (dd, J = 13.4, 10.3 Hz, 0.5H), 3.11 (m, 0.5H), 3.03-2.71 (m, 2H), 2.22 (d, J = 12.8 Hz, 1H), 2.05 (d, J = 1.7 Hz, 3H), 1.99- 1.72 (m, 2H), 1.70-1.38 (m, 1H); LCMS (Method D): t_{R} 2.97 min, 100%, MS (ESI) 376.2 (M + H)⁺ |
| 00299 | 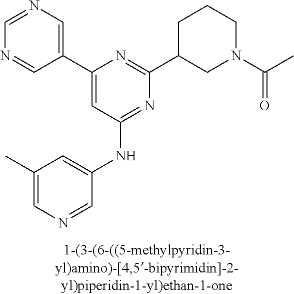 [SMILES: CC(=O)N1CCCC(c2nc(Nc3cncc(C)c3)cc(-c3cncnc3)n2)C1] | ¹H-NMR (400 MHz, DMSO-d6) mixture of rotamers δ 10.02 (d, J = 6.4 Hz, 1H), 9.39 (d, J = 7.4 Hz, 2H), 9.33 (d, J = 2.4 Hz, 1H), 8.74-8.68 (m, 1H), 8.14-8.05 (m, 2H), 7.24 (d, J = 6.7 Hz, 1H), 4.78-4.71 (m, 0.5H), 4.21 (d, J = 12.9 Hz, 0.5H), 4.18-4.09 (m, 0.5H), 3.84 (d, J = 13.5 Hz, 0.5H), 3.55-3.47 (m, 0.5H), 3.09 (t, J = 12.0 Hz, 0.5H), 3.01-2.85 (m, 1H), 2.83- 2.73 (m, 1H), 2.33 (d, J = 3.5 Hz, 3H), 2.28-2.18 (m, 1H), 2.10-2.00 (m, 3H), 1.96-1.72 (m, 2H), 1.67-1.40 (m, 1H); LCMS (Method D): t_{R} 2.89 min, 99%, MS (ESI) 390.1 (M + H)⁺ |
| 00300 | 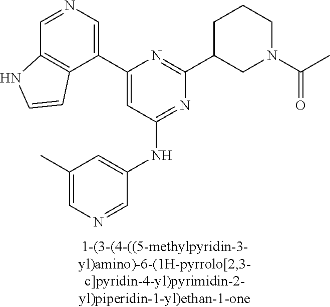 [SMILES: CC(=O)N1CCCC(c2nc(Nc3cncc(C)c3)cc(-c3cncc4[nH]ccc34)n2)C1] | ¹H-NMR (400 MHz, DMSO-d6) mixture of rotamers δ 11.93 (s, 1H), 9.83 (s, 1H), 8.87 (s, 1H), 8.76-8.69 (m, 2H), 8.18-8.12 (m, 1H), 8.12-8.06 (m, 1H), 7.81 (t, J = 3.0 Hz, 1H), 7.29 (d, J = 5.4 Hz, 1H), 7.05 (d, J = 3.3 Hz, 1H), 4.86 (d, J = 10.0 Hz, 0.5H), 4.31- 4.15 (m, 1H), 3.87 (d, J = 13.4 Hz, 0.5H), 3.55-3.45 (m, 0.5H), 3.14- 3.05 (m, 0.5H), 3.05-2.94 (m, 0.5H), 2.90-2.65 (m, 1.5H), 2.36-2.22 (m, 4H), 2.10-2.03 (m, 3H), 1.99-1.74 (m, 2H), 1.67-1.39 (m, 1H); LCMS (Method D): t_{R} 3.01 min, 98%, MS (ESI) 428.2 (M + H)⁺ |
| 00301 | 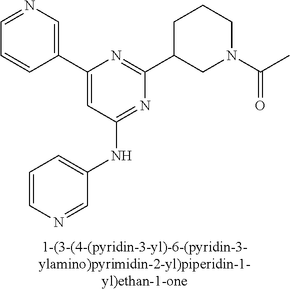 [SMILES: CC(=O)N1CCCC(c2nc(Nc3cccnc3)cc(-c3cccnc3)n2)C1] | ¹H-NMR (400 MHz, DMSO-d6) mixture of rotamers δ 9.99 (s, 1H), 9.22 (dd, J = 5.5, 2.3 Hz, 1H), 8.92 (d, J = 2.5 Hz, 1H), 8.81-8.66 (m, 1H), 8.52-8.36 (m, 1H), 8.36-8.15 (m, 2H), 7.58 (dd, J = 8.1, 4.8 Hz, 1H), 7.47-7.32 (m, 1H), 7.20 (d, J = 4.8 Hz, 1H), 4.77- 4.69 (m, 0.5H), 4.23 (d, J = 12.6 Hz, 0.5H), 4.19-4.05 (m, 0.5H), 3.94- 3.79 (m, 0.5H), 3.51 (dd, J = 13.5, 10.2 Hz, 0.5H), 3.17-3.02 (m, 0.5H), 3.01-2.70 (m, 2H), 2.29-2.12 (m, 1H), 2.04 (d, J = 4.1 Hz, 3H), 1.97- 1.69 (m, 2H), 1.67-1.38 (m, 1H); LCMS (Method D): t_{R} 2.89 min, 97%, MS (ESI) 375.1 (M + H)⁺ |
| 00302 | 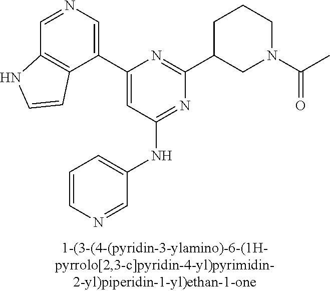 [SMILES: CC(=O)N1CCCC(c2nc(Nc3cccnc3)cc(-c3cncc4[nH]ccc34)n2)C1] | ¹H-NMR (400 MHz, DMSO-d6) mixture of rotamers δ 11.93 (s, 1H), 9.91 (d, J = 8.4 Hz, 1H), 8.93 (d, J = 2.7 Hz, 1H), 8.86 (s, 1H), 8.73 (d, J = 4.1 Hz, 1H), 8.37-8.17 (m, 2H), 7.80 (t, J = 2.7 Hz, 1H), 7.40 (dt, J = 8.3, 4.1 Hz, 1H), 7.30 (d, J = 4.1 Hz, 1H), 7.06 (d, J = 3.0 Hz, 1H), 4.81 (d, J = 11.8 Hz, 0.5H), 4.27 (d, J = 12.9 Hz, 0.5H), 4.17 (d, J = 14.4 Hz, 1H), 3.87 (d, J = 13.5 Hz, 0.5H), 3.51 (dd, J = 13.4, 10.4 Hz, 0.5H), 3.24-3.05 (m, 0.5H), 3.06-2.93 (m, 0.5H), 2.93-2.63 (m, 1.5H), 2.27 (d, J = 12.3 Hz, 1H), 2.05 (d, J = 2.2 Hz, 3H), 1.98-1.72 (m, 2H), 1.56 (dt, J = 50.8, 13.4 Hz, 1H); LCMS (Method D): tR 2.88 min, 94%, MS (ESI) 414.2 (M + H)+ |
| 00303 | 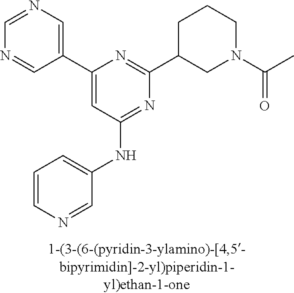 [SMILES: CC(=O)N1CCCC(c2nc(Nc3cccnc3)cc(-c3cncnc3)n2)C1] | ¹H-NMR (400 MHz, DMSO-d6) mixture of rotamers δ 10.08 (s, 1H), 9.40 (d, J = 7.3 Hz, 2H), 9.33 (d, J = 2.3 Hz, 1H), 8.91 (d, J = 2.6 Hz, 1H), 8.30-8.17 (m, 2H), 7.40 (m, 1H), 7.25 (d, J = 6.0 Hz, 1H), 4.75-4.63 (m, 0.5H), 4.22 (d, J = 13.1 Hz, 0.5H), 4.11 (d, J = 13.8 Hz, 0.5H), 3.84 (d, J = 13.4 Hz, 0.5H), 3.52 (dd, J = 13.5, 10.2 Hz, 0.5H), 3.10 (m, 0.5H), 3.02-2.86 (m, 0.5H), 2.85-2.71 (m, 1H), 2.21 (d, J = 13.0 Hz, 1H), 2.03 (d, J = 3.1 Hz, 3H), 1.94-1.68 (m, 2H), 1.68-1.39 (m, 1H); LCMS (Method D): tR 2.75 min, 98%, MS (ESI) 376.2 (M + H)+ |
| 00304 | 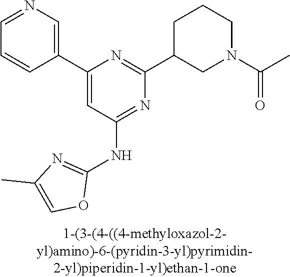 [SMILES: CC(=O)N1CCCC(c2nc(Nc3nc(C)co3)cc(-c3cccnc3)n2)C1] | ¹H-NMR (400 MHz, DMSO-d6) mixture of rotamers δ 11.38 (s, 1H), 9.23 (m, 1H), 8.74 (m, 1H), 8.48-8.32 (m, 2H), 7.61 (dd, J = 7.9, 4.8 Hz, 1H), 7.52 (m, 1H), 4.73 (dd, J = 12.1, 2.9 Hz, 0.5H), 4.23 (d, J = 12.9 Hz, 0.5H), 4.08 (dd, J = 13.5, 3.8 Hz, 0.5H), 3.84 (s, 0.5H), 3.56 (dd, J = 13.5, 10.2 Hz, 0.5H), 3.07 (m, 0.5H), 3.00-2.89 (m, 0.5H), 2.90-2.71 (m, 1.5H), 2.12 (m, 4H), 2.04 (d, J = 5.6 Hz, 3H), 1.97-1.69 (m, 2H), 1.64-1.37 (m, 1H); LCMS (Method D): t_{R} 3.07 min, 100%, MS (ESI) 379.1 (M + H)⁺ |
| 00305 | 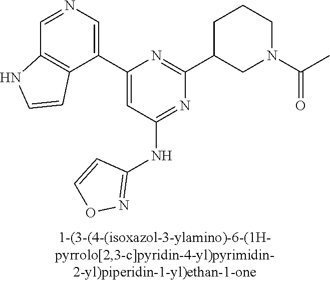 [SMILES: CC(=O)N1CCCC(c2nc(Nc3ccon3)cc(-c3cncc4[nH]ccc34)n2)C1] | ¹H-NMR (400 MHz, DMSO-d6) mixture of rotamers δ 11.94 (s, 1H), 10.63 (s, 1H), 8.91 (s, 1H), 8.81 (d, J = 1.7 Hz, 1H), 8.76 (d, J = 3.2 Hz, 1H), 7.88- 7.79 (m, 2H), 7.10-7.03 (m, 1H), 6.90 (s, 1H), 4.82 (d, J = 9.3 Hz, 0.5H), 4.29 (d, J = 12.9 Hz, 0.5H), 4.16 (dd, J = 13.2, 3.9 Hz, 0.5H), 3.87 (d, J = 13.8 Hz, 0.5H), 3.52 (dd, J = 13.5, 10.5 Hz, 0.5H), 3.15-3.04 (m, 0.5H), 3.04-2.95 (m, 0.5H), 2.90- 2.80 (m, 1H), 2.80-2.68 (m, 0.5H), 2.31-2.20 (m, 1H), 2.05 (s, 3H), 1.97- 1.73 (m, 2H), 1.67-1.41 (m, 1H); LCMS (Method D): tR 2.94 min, 100%, MS (ESI) 404.1 (M + H)+ |
| 00306 | 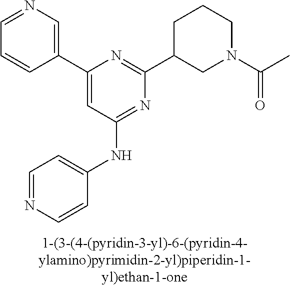 [SMILES: CC(=O)N1CCCC(c2nc(Nc3ccncc3)cc(-c3cccnc3)n2)C1] | 1H-NMR (400 MHz, DMSO-d6) mixture of rotamers δ 10.22 (s, 1H), 9.23 (m, 1H), 8.75-8.70 (m, 1H), 8.47-8.37 (m, 3H), 7.81-7.75 (m, 2H), 7.59 (m, 1H), 7.27 (d, J = 4.5 Hz, 1H), 4.78-4.71 (m, 0.5H), 4.28-4.20 (m, 0.5H), 4.18-4.11 (m, 0.5H), 3.90- 3.83 (m, 0.5H), 3.55 (dd, J = 13.5, 10.2 Hz, 0.5H), 3.16-3.07 (m, 0.5H), 3.06-2.97 (m, 0.5H), 2.97-2.88 (m, 0.5H), 2.88-2.73 (m, 1H), 2.29-2.19 (m, 1H), 2.05 (s, 3H), 1.96-1.73 (m, 2H), 1.68-1.40 (m, 1H); LCMS (Method D): tR 2.87 min, 100%, MS (ESI) 375.1 (M + H)+ |
| 00307 | 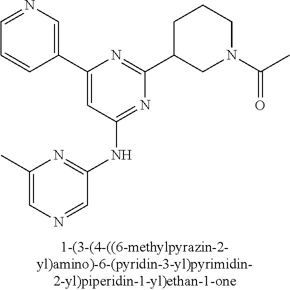 [SMILES: CC(=O)N1CCCC(c2nc(Nc3cncc(C)n3)cc(-c3cccnc3)n2)C1] | ¹H-NMR (400 MHz, DMSO-d6) mixture of rotamers δ 10.59 (s, 1H), 9.22 (dd, J = 4.8, 2.2 Hz, 1H), 9.09 (d, J = 5.6 Hz, 1H), 8.73 (dt, J = 4.9, 1.7 Hz, 1H), 8.40 (m, 1H), 8.17 (s, 1H), 7.98 (s, 1H), 7.60 (dd, J = 8.0, 4.8 Hz, 1H), 4.76 (d, J = 11.5 Hz, 0.5H), 4.25 (d, J = 13.0 Hz, 0.5H), 4.13 (dd, J = 13.7, 3.2 Hz, 0.5H), 3.87 (d, J = 13.4 Hz, 0.5H), 3.54 (dd, J = 13.4, 10.3 Hz, 0.5H), 3.14-3.04 (m, 0.5H), 3.04- 2.95 (m, 0.5H), 2.93-2.72 (m, 1.5H), 2.48 (s, 3H), 2.28-2.18 (m, 1H), 2.05 (d, J = 3.0 Hz, 3H), 1.96-1.74 (m, 2H), 1.66-1.41 (m, 1H); LCMS (Method D): tR 3.04 min, 100%, MS (ESI) 390.1 (M + H)+ |
| 00308 | 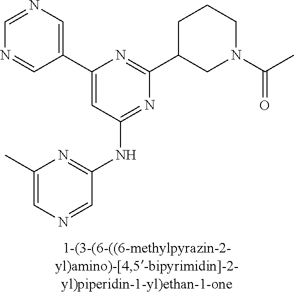 [SMILES: CC(=O)N1CCCC(c2nc(Nc3cncc(C)n3)cc(-c3cncnc3)n2)C1] | ¹H-NMR (400 MHz, DMSO-d6) mixture of rotamers δ 10.67 (d, J = 4.2 Hz, 1H), 9.41-9.29 (m, 3.1H), 9.06 (d, J = 6.9 Hz, 1H), 8.44-8.35 (m, 0.1H), 8.21-8.14 (m, 1H), 8.03 (s, 1H), 4.77- 4.70 (m, 0.5H), 4.28-4.22 (m, 0.5H), 4.17-4.09 (m, 0.5H), 3.89- 3.81 (m, 0.5H), 3.60-3.50 (m, 0.5H), 3.14-3.07 (m, 0.5H), 3.05-2.96 (m, 0.5H), 2.95-2.88 (m, 0.5H), 2.88- 2.72 (m, 1H), 2.55-2.53 (m, 3H), 2.28-2.18 (m, 1H), 2.09-2.02 (m, 3H), 1.96-1.74 (m, 2H), 1.66-1.41 (m, 1H); LCMS (Method D): tR 2.88 min, 100%, MS (ESI) 391.1 (M + H)+ |
| 00309 | 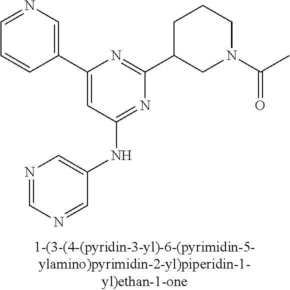 [SMILES: CC(=O)N1CCCC(c2nc(Nc3cncnc3)cc(-c3cccnc3)n2)C1] | ¹H-NMR (400 MHz, Chloroform-d)) mixture of rotamers δ 9.23-9.08 (m, 3H), 8.94 (m, 1H), 8.71 (m, 1H), 8.34 (m, 1H), 7.95 (s, 0.5H), 7.49-7.35 (m, 1.5H), 7.00 (m, 1H), 4.95-4.87 (m, 0.5H), 4.50-4.41 (m, 0.5H), 4.12 (dd, J = 13.2, 3.9 Hz, 0.5H), 3.88 (d, J = 13.5 Hz, 0.5H), 3.60 (dd, J = 13.4, 10.3 Hz, 0.5H), 3.18 (m, 0.5H), 3.10- 2.99 (m, 1H), 2.96-2.81 (m, 1H), 2.35-2.26 (m, 1H), 2.17 (m, 3H), 2.02-1.75 (m, 2H), 1.73-1.63 (m, 1H); LCMS (Method D): tR 2.75 min, 99%, MS (ESI) 376.1 (M + H)+ |
| 00310 | 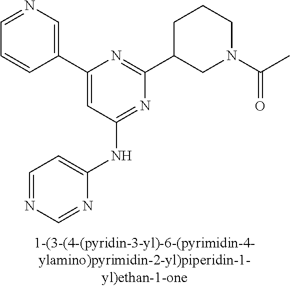 [SMILES: CC(=O)N1CCCC(c2nc(Nc3ccncn3)cc(-c3cccnc3)n2)C1] | ¹H-NMR (400 MHz, Chloroform-d)) mixture of rotamers δ 10.76 (s, 1H), 9.24 (m, 1H), 8.88 (s, 1H), 8.74 (m, 1H), 8.59 (m, 1H), 8.42 (m, 1H), 8.15 (s, 1H), 7.90 (t, J = 6.1 Hz, 1H), 7.61 (m, 1H), 4.75 (d, J = 12.0 Hz, 0.5H), 4.26 (d, J = 12.8 Hz, 0.5H), 4.13 (d, J = 14.5 Hz, 0.5H), 3.86 (d, J = 13.5 Hz, 0.5H), 3.56 (dd, J = 13.5, 10.4 Hz, 0.5H), 3.16-3.06 (m, 0.5H), 3.06- 2.95 (m, 0.5H), 2.96-2.72 (m, 1.5H), 2.27-2.18 (m, 1H), 2.05 (d, J = 2.3 Hz, 3H), 1.96-1.74 (m, 2H), 1.66- 1.42 (m, 1H); LCMS (Method D): tR 2.73 min, 96%, MS (ESI) 376.2 (M+)+ |
| 00311 | 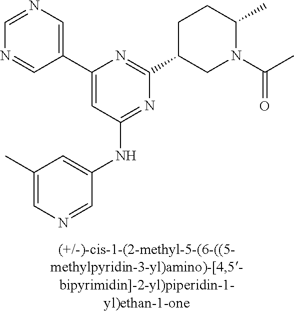 [SMILES: CC(=O)N1C[C@H](c2nc(Nc3cncc(C)c3)cc(-c3cncnc3)n2)CC[C@@H]1C] | ¹H-NMR (400 MHz, DMSO-d6) mixture of rotamers δ 10.04 (s, 1H), 9.39 (m, 2H), 9.33 (d, J = 1.9 Hz, 1H), 8.70 (m, 1H), 8.20-8.05 (m, 2H), 7.24 (m, H), 4.83 (s, 0.5H), 4.74 (dd, J = 13.2, 4.1 Hz, 0.5H), 4.27-4.15 (m, 0.5H), 4.03 (m, 0.5H), 3.44 (dd, J = 13.7, 11.8 Hz, 0.5H), 2.90 (m, 1H), 2.75 (m, 0.5H), 2.33 (m, 3H), 2.05 (m, 5H), 1.89- 1.63 (m, 2H), 1.26 (m, 1.5H), 1.15 (m, 1.5H); LCMS (Method D): t_{R} 2.99 min, 95%, MS (ESI) 404.1 (M + H)⁺ |
| 00312 | 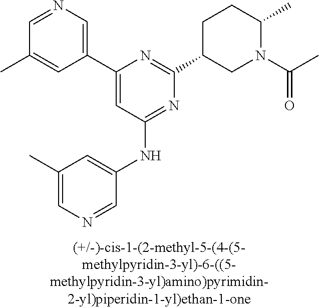 [SMILES: CC(=O)N1C[C@H](c2nc(Nc3cncc(C)c3)cc(-c3cncc(C)c3)n2)CC[C@@H]1C] | ¹H-NMR (400 MHz, DMSO-d6) mixture of rotamers δ 9.91 (s, 1H), 9.00 (m, 1H), 8.69 (m, 1H), 8.55 (s, 1H), 8.24- 8.14 (m, 2H), 8.09 (t, J = 2.3 Hz, 1H), 7.18 (d, J = 4.5 Hz, 1H), 4.82 (d, J = 7.3 Hz, 0.5H), 4.74 (m, 0.5H), 4.22 (m, 0.5H), 4.02 (dd, J = 13.4, 4.2 Hz, 0.5H), 3.44 (dd, J = 13.8, 11.9 Hz, 0.5H), 2.94-2.82 (m, 1H), 2.80-2.65 (m, 0.5H), 2.41 (s, 3H), 2.32 (m, 3H), 2.05 (m, 5H), 1.90-1.60 (m, 2H), 1.32-1.24 (m, 1.5H), 1.19-1.12 (m, 1.5H); LCMS (Method D): t_{R} 3.20 min, 94%, MS (ESI) 417.2 (M + H)⁺ |
| 00313 | 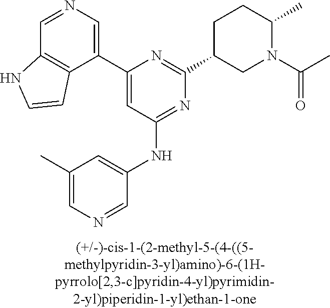 [SMILES: CC(=O)N1C[C@H](c2nc(Nc3cncc(C)c3)cc(-c3cncc4[nH]ccc34)n2)CC[C@@H]1C] | ¹H-NMR (400 MHz, DMSO-d6) mixture of rotamers δ 11.93 (s, 1H), 9.89 (s, 1H), 8.86 (s, 1H), 8.78-8.67 (m, 2H), 8.21 (m, 1H), 8.09 (d, J = 2.7 Hz, 1H), 7.81 (t, J = 2.7 Hz, 1H), 7.30 (d, J = 2.8 Hz, 1H), 7.08 (d, J = 2.8 Hz, 1H), 4.89-4.76 (m, 1H), 4.29-4.18 (m, 0.5H), 4.08 (m, 0.5H), 3.53-3.48 (m, 0.5H), 3.01-2.87 (m, 1H), 2.84-2.71 (m, 0.5H), 2.34 (m, 3H), 2.18-1.97 (m, 5H), 1.95-1.64 (m, 2H), 1.32- 1.24 (m, 1.5H), 1.19-1.12 (m, 1.5H);; LCMS (Method D): t_{R} 3.10 min, 94%, MS (ESI) 442.1 (M + H)⁺ |
| 00314 | 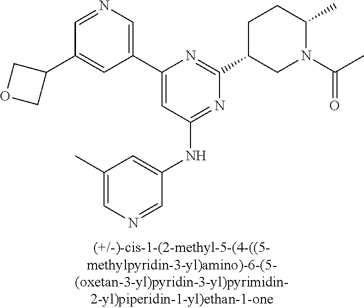 [SMILES: CC(=O)N1C[C@H](c2nc(Nc3cncc(C)c3)cc(-c3cncc(C4COC4)c3)n2)CC[C@@H]1C] | ¹H-NMR (400 MHz, DMSO-d6) mixture of rotamers δ 9.95 (s, 1H), 9.13 (dd, J = 6.8, 2.1 Hz, 1H), 8.79-8.63 (m, 2H), 8.47 (dt, J = 4.1, 2.1 Hz, 1H), 8.19 (dd, J = 15.1, 2.4 Hz, 1H), 8.09 (d, J = 2.3 Hz, 1H), 7.25 (d, J = 4.8 Hz, 1H), 5.09-4.97 (m, 2H), 4.87-4.81 (m, 0.5H), 4.78-4.64 (m, 2.5H), 4.47- 4.36 (m, 1H), 4.29-4.18 (m, 0.5H), 4.13-3.97 (m, 0.5H), 3.55-3.41 (m, 0.5H), 3.01-2.83 (m, 1H), 2.82-2.70 (m, 0.5H), 2.33 (d, J = 3.6 Hz, 3H), 2.05 (m, 5H), 1.93-1.61 (m, 2H), 1.32-1.24 (m, 1.5H), 1.19-1.12 (m, 1.5H); LCMS (Method D): tR 2.99 min, 98%, MS (ESI) 459.4 (M + H)+ |
| 00315 | 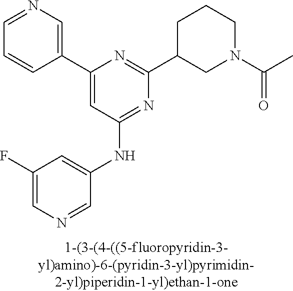 [SMILES: CC(=O)N1CCCC(c2nc(Nc3cncc(F)c3)cc(-c3cccnc3)n2)C1] | ¹H-NMR (400 MHz, DMSO-d6) mixture of rotamers δ 10.16 (s, 1H), 9.23 (dd, J = 5.8, 2.2 Hz, 1H), 8.72 (d, J = 4.8 Hz, 1H), 8.67 (s, 1H), 8.45-8.32 (m, 2H), 8.22 (d, J = 2.7 Hz, 1H), 7.58 (dd, J = 8.0, 4.8 Hz, 1H), 7.23 (d, J = 4.4 Hz, 1H), 4.79-4.70 (m, 0.5H), 4.24 (d, J = 12.8 Hz, 0.5H), 4.19-4.10 (m, 0.5H), 3.85 (d, J = 13.5 Hz, 0.5H), 3.53-3.46 (m, 0.5H), 3.14-3.04 (m, 0.5H), 3.04-2.71 (m, 2H), 2.29-2.18 (m, 1H), 2.04 (s, 3H), 1.95-1.71 (m, 2H), 1.70-1.41 (m, 1H); LCMS (Method D): tR 3.08 min, 99%, MS (ESI) 393.2 (M + H)+ |
| 00316 | 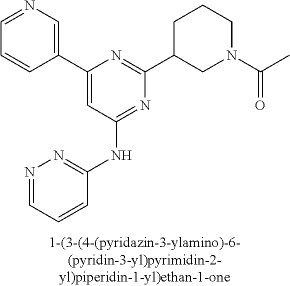 [SMILES: CC(=O)N1CCCC(c2nc(Nc3cccnn3)cc(-c3cccnc3)n2)C1] | ¹H-NMR (400 MHz, DMSO-d6) mixture of rotamers δ 10.77 (s, 1H), 9.23 (dd, J = 4.6, 2.3 Hz, 1H), 8.91 (dt, J = 4.8, 1.7 Hz, 1H), 8.74 (dd, J = 4.1, 2.3 Hz, 1H), 8.40 (dq, J = 7.3, 2.3 Hz, 1H), 8.27 (t, J = 7.9 Hz, 1H), 8.07- 7.96 (m, 1H), 7.74-7.66 (m, 1H), 7.61 (dd, J = 8.1, 4.8 Hz, 1H), 4.73 (d, J = 12.4 Hz, 0.5H), 4.26 (d, J = 13.0 Hz, 0.5H), 4.12 (d, J = 13.9 Hz, 0.5H), 3.86 (d, J = 13.5 Hz, 0.5H), 3.54 (dd, J = 13.4, 10.4 Hz, 0.5H), 3.18-3.06 (m, 0.5H), 3.04-2.70 (m, 2H), 2.22 (d, J = 13.0 Hz, 1H), 2.04 (d, J = 2.5 Hz, 3H), 1.96-1.70 (m, 2H), 1.67-1.37 (m, 1H); LCMS (Method D): tR 2.34 min, 99%, MS (ESI) 376.1 (M + H)+ |
| 00317 | 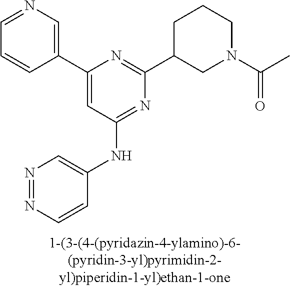 [SMILES: CC(=O)N1CCCC(c2nc(Nc3ccnnc3)cc(-c3cccnc3)n2)C1] | 1H-NMR (400 MHz, DMSO-d6) mixture of rotamers δ 10.58-10.39 (m, 1H), 9.44 (d, J = 2.7 Hz, 1H), 9.25 (dd, J = 5.6, 2.2 Hz, 1H), 9.02 (t, J = 5.4 Hz, 1H), 8.78-8.69 (m, 1H), 8.49- 8.39 (m, 1H), 8.24-8.17 (m, 1H), 7.60 (dd, J = 8.1, 4.8 Hz, 1H), 7.33 (d, J = 4.8 Hz, 1H), 4.79-4.68 (m, 0.5H), 4.26 (d, J = 12.9 Hz, 0.5H), 4.19- 4.09 (m, 0.5H), 3.91-3.81 (m, 0.5H), 3.56 (dd, J = 13.4, 10.3 Hz, 0.5H), 3.19-2.74 (m, 2.5H), 2.30-2.18 (m, 1H), 2.05 (s, 3H), 1.96-1.73 (m, 2H), 1.71-1.43 (m, 1H); LCMS (Method D): tR 2.10 min, 99%, MS (ESI) 376.1 (M + H)+ |
| 00318 | 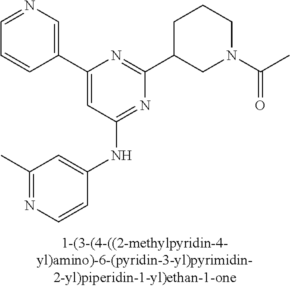 [SMILES: CC(=O)N1CCCC(c2nc(Nc3ccnc(C)c3)cc(-c3cccnc3)n2)C1] | 1H-NMR (400 MHz, DMSO-d6) mixture of rotamers δ 10.12 (d, J = 8.3 Hz, 1H), 9.22 (dd, J = 5.7, 2.3 Hz, 1H), 8.77-8.66 (m, 1H), 8.45-8.36 (m, 1H), 8.30 (dd, J = 5.7, 3.6 Hz, 1H), 7.68 (dd, J = 12.6, 2.1 Hz, 1H), 7.62- 7.53 (m, 2H), 7.25 (d, J = 5.2 Hz, 1H), 4.86-4.74 (m, 0.5H), 4.29-4.20 (m, 0.5H), 4.15 (dd, J = 13.5, 3.9 Hz, 0.5H), 3.92-3.80 (m, 0.5H), 3.54 (dd, J = 13.5, 10.1 Hz, 0.5H), 3.16-3.06 (m, 0.5H), 3.06-2.95 (m, 0.5H), 2.93- 2.74 (m, 1.5H), 2.45 (s, 3H), 2.32- 2.19 (m, 1H), 2.05 (s, 3H), 1.98-1.72 (m, 2H), 1.68-1.40 (m, 1H); LCMS (Method D): tR 2.94 min, 99%, MS (ESI) 389.2 (M + H)+ |
| 00319 | 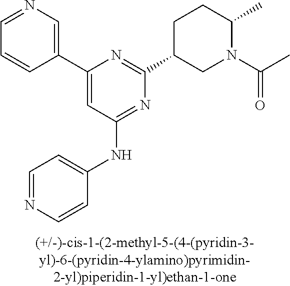 [SMILES: CC(=O)N1C[C@H](c2nc(Nc3ccncc3)cc(-c3cccnc3)n2)CC[C@@H]1C] | 1H-NMR (400 MHz, DMSO-d6) mixture of rotamers δ 10.23 (s, 1H), 9.24 (s, 1H), 8.73 (d, J = 4.7 Hz, 1H), 8.50-8.34 (m, 3H), 7.90-7.73 (m, 2H), 7.59 (dd, J = 8.0, 4.7 Hz, 1H), 7.28 (d, J = 3.8 Hz, 1H), 4.84 (s, 0.5H), 4.79-4.70 (m, 0.5H), 4.32- 4.15 (m, 0.5H), 4.13-4.02 (m, 0.5H), 3.56-3.43 (m, 0.5H), 3.03-2.86 (m, 1H), 2.85-2.72 (m, 0.5H), 2.17-1.61 (m, 7H), 1.33-1.26 (m, 1.5H), 1.21- 1.11 (m, 1.5H); LCMS (Method D): tR 2.98 min, 97%, MS (ESI) 389.2 (M + H)+ |
| 00320 | 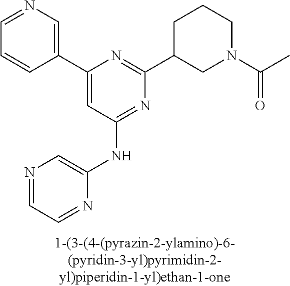 [SMILES: CC(=O)N1CCCC(c2nc(Nc3cnccn3)cc(-c3cccnc3)n2)C1] | 1H-NMR (400 MHz, DMSO-d6) mixture of rotamers δ 10.65 (s, 1H), 9.26-9.19 (m, 2H), 8.76-8.71 (m, 1H), 8.44-8.38 (m, 2H), 8.27 (t, J = 2.4 Hz, 1H), 8.04 (s, 1H), 7.60 (dd, J = 7.9, 4.9 Hz, 1H), 4.77 (d, J = 11.2 Hz, 0.5H), 4.30-4.23 (m, 0.5H), 4.17- 4.10 (m, 0.5H), 3.90-3.83 (m, 0.5H), 3.62-3.50 (m, 0.5H), 3.14-3.05 (m, 0.5H), 3.04-2.95 (m, 0.5H), 2.94- 2.72 (m, 1.5H), 2.28-2.18 (m, 1H), 2.05 (m, 3H), 1.96-1.74 (m, 2H), 1.67-1.41 (m, 1H); LCMS (Method D): tR 2.88 min, 98%, MS (ESI) 376.2 (M + H)+ |
| 00321 | 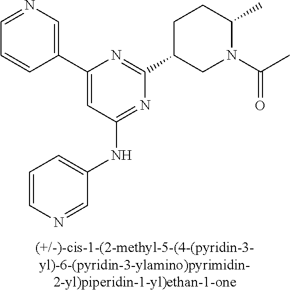 [SMILES: CC(=O)N1C[C@H](c2nc(Nc3cccnc3)cc(-c3cccnc3)n2)CC[C@@H]1C] | ¹H-NMR (400 MHz, DMSO-d6) mixture of rotamers δ 9.99 (s, 1H), 9.22 (dd, J = 6.4, 2.3 Hz, 1H), 8.94 (d, J = 2.5 Hz, 1H), 8.72 (d, J = 4.7 Hz, 1H), 8.46- 8.32 (m, 1H), 8.32-8.17 (m, 2H), 7.58 (dd, J = 8.1, 4.7 Hz, 1H), 7.45- 7.34 (m, 1H), 7.20 (d, J = 4.0 Hz, 1H), 4.82 (s, 0.5H), 4.72 (dd, J = 13.4, 4.2 Hz, 0.5H), 4.27-4.16 (m, 0.5H), 4.04 (dd, J = 13.9, 4.2 Hz, 0.5H), 3.45 (dd, J = 13.6, 11.9 Hz, 0.5H), 2.99-2.84 (m, 1H), 2.79-2.69 (m, 0.5H), 2.10- 1.62 (m, 7H), 1.32-1.22 (m, 1.5H), 1.19-1.10 (m, 1.5H); LCMS (Method D): tR 2.99 min, 100%, MS (ESI) 389.2 (M + H)+ |

### Example 42: Synthesis of 1-(3-(4-(cyclohexylamino)-6-(pyridin-3-yl)pyrimidin-2-yl)piperidin-1-yl)ethan-1-one 00322

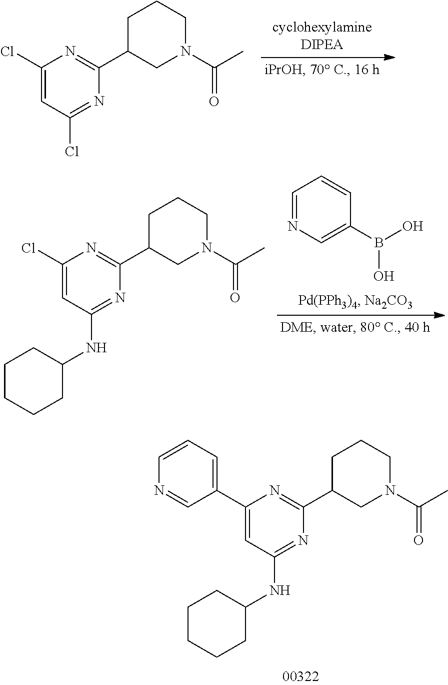

A solution of 1-(3-(4,6-dichloropyrimidin-2-yl)piperidin-1-yl)ethan-1-one (50 mg, 0.18 mmol), cyclohexylamine (0.021 mL, 0.18 mmol) and diisopropylethylamine (0.038 mL, 0.22 mmol) in isopropanol (2 mL) was heated at 70° C. for 16 hours. The mixture was concentrated and purified by using reversed phase chromatography (Method B) to afford 1-(3-(4-chloro-6-(cyclohexylamino)pyrimidin-2-yl)piperidin-1-yl)ethan-1-one (48 mg, 78%) as a white solid. LCMS (Method A): t_{R} 2.01 min, 100%, MS (ESI) 337.2 (M+H)⁺. To a N₂-degassed mixture of 1-(3-(4-chloro-6-(cyclohexylamino)pyrimidin-2-yl)piperidin-1-yl)ethan-1-one (48 mg, 0.14 mmol), sodiumcarbonate (2M, 0.21 mL, 0.42 mmol) and 1,2-dimethoxyethane (1 mL) was added palladium tetrakis triphenylphosphine (8.2 mg, 7.1 μmol) and pyridin-3-ylboronic acid (44 mg, 0.36 mmol) and the reaction mixture was stirred at 80° C. for 16 hours. Additional palladium tetrakis triphenylphosphine (8.2 mg, 7.1 μmol) and pyridin-3-ylboronic acid (44 mg, 0.36 mmol) was added and the reaction mixture was stirred at 80° C. for an additional 24 hours. The reaction mixture was diluted with dichloromethane and washed with water. The organic layer was concentrated and the crude product was purified by using reversed phase chromatography (Method B) to afford 1-(3-(4-(cyclohexylamino)-6-(pyridin-3-yl)pyrimidin-2-yl)piperidin-1-yl)ethan-1-one (10 mg, 18%) as a white solid. ¹H-NMR (400 MHz, DMSO-d6) mixture of rotamers δ 9.12 (s, 1H), 8.65 (dd, J=4.8, 1.7 Hz, 1H), 8.30 (s, 1H), 7.55-7.46 (m, 1H), 7.33 (s, 1H), 6.82 (s, 1H), 4.64 (d, J=12.6 Hz, 0.5H), 4.14 (s, 0.5H), 4.05-3.75 (m, 2H), 3.55-3.42 (m, 0.5H), 3.04 (t, J=12.7 Hz, 0.5H), 2.78 (s, 1.5H), 2.61 (d, J=11.2 Hz, 0.5H), 2.20-2.06 (m, 1H), 2.01 (d, J=6.4 Hz, 3H), 1.97-1.67 (m, 6H), 1.63-1.13 (m, 7H); LCMS (Method B): t_{R} 2.52 min, 98%, MS (ESI) 380.2 (M+H)⁺.

The following compounds were prepared using procedures analogous to Example 42.

| Compound # | Structure and compound name | Analytical data |
|---|---|---|
| 00323 | 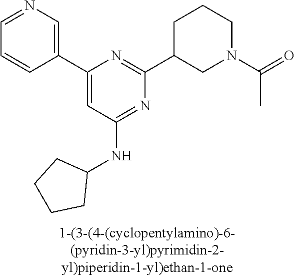 [SMILES: CC(=O)N1CCCC(c2nc(NC3CCCC3)cc(-c3cccnc3)n2)C1] | ¹H-NMR (400 MHz, DMSO-d6) mixture of rotamers δ 9.14 (s, 1H), 8.70-8.61 (m, 1H), 8.32 (s, 1H), 7.57-7.40 (m, 2H), 6.83 (s, 1H), 4.74-4.58 (m, 0.5H), 4.23 (d, J = 48.5 Hz, 1.5H), 4.00 (dd, J = 13.5, 3.9 Hz, 0.5H), 3.83 (d, J = 13.1 Hz, 0.5H), 3.50 (dd, J = 13.5, 10.0 Hz, 0.5H), 3.10-2.98 (m, 0.5H), 2.79 (d, J = 12.0 Hz, 1.5H), 2.66-2.55 (m, 0.5H), 2.20-2.07 (m, 1H), 1.99 (dd, J = 17.1, 6.7 Hz, 5H), 1.88-1.38 (m, 9H); LCMS (Method B): t_{R} 2.40 min, 100%, MS (ESI) 366.2 (M + H)⁺ |
| 00324 | 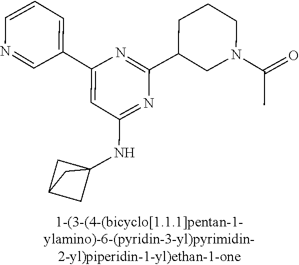 [SMILES: CC(=O)N1CCCC(c2nc(NC34CC(C3)C4)cc(-c3cccnc3)n2)C1] | ¹H-NMR (400 MHz, DMSO-d6) mixture of rotamers δ 9.14 (s, 1H), 8.75-8.58 (m, 1H), 8.44-8.22 (m, 1H), 8.13 (d, J = 7.9 Hz, 1H), 7.59-7.45 (m, 1H), 6.80 (d, J = 4.7 Hz, 1H), 4.78-4.63 (m, 0.5H), 4.29-4.13 (m, 0.5H), 4.11- 3.99 (m, 0.5H), 3.84 (d, J = 13.3 Hz, 0.5H), 3.45 (dd, J = 13.5, 10.2 Hz, 0.5H), 3.10-2.97 (m, 0.5H), 2.87- 2.58 (m, 2H), 2.52-2.51 (m, 1H), 2.23-2.09 (m, 7H), 2.02 (d, J = 2.9 Hz, 3H), 1.92-1.68 (m, 2H), 1.62- 1.35 (m, 1H); LCMS (Method B): t_{R} 2.49 min, 99%, MS (ESI) 364.2 (M + H)⁺ |
| 00325 | 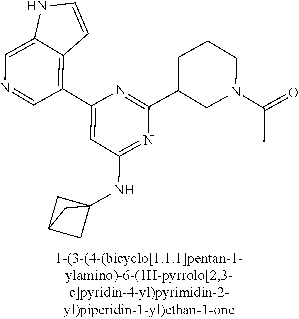 [SMILES: CC(=O)N1CCCC(c2nc(NC34CC(C3)C4)cc(-c3cncc4[nH]ccc34)n2)C1] | ¹H-NMR (400 MHz, DMSO-d6) mixture of rotamers δ 1H NMR (400 MHz, DMSO-d6) δ 11.87 (s, 1H), 8.81 (s, 1H), 8.68-8.54 (m, 1H), 8.15-7.96 (m, 1H), 7.81-7.65 (m, 1H), 6.98- 6.93 (m, 1H), 6.93-6.85 (m, 1H), 4.83-4.73 (m, 0.5H), 4.31-4.20 (m, 0.5H), 4.17-4.06 (m, 0.5H), 3.90- 3.80 (m, 0.5H), 3.49-3.40 (m, 0.5H), 3.09-2.97 (m, 0.5H), 2.90-2.61 (m, 2H), 2.27-2.10 (m, 7H), 2.08-1.96 (m, 4H), 1.91-1.69 (m, 3H), 1.62- 1.38 (m, 1H); LCMS (Method D): t_{R} 3.33 min, 98%, MS (ESI) 403.2 (M + H)⁺ |
| 00326 | 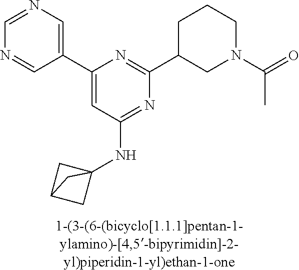 [SMILES: CC(=O)N1CCCC(c2nc(NC34CC(C3)C4)cc(-c3cncnc3)n2)C1] | ¹H-NMR (400 MHz, DMSO-d6) mixture of rotamers δ 9.45-9.19 (m, 3H), 8.32-8.21 (m, 1H), 6.86 (s, 1H), 4.74- 4.64 (m, 0.5H), 4.26-4.15 (m, 0.5H), 4.11-3.99 (m, 0.5H), 3.89- 3.78 (m, 0.5H), 3.50-3.41 (m, 0.5H), 3.10-3.00 (m, 0.5H), 2.88-2.60 (m, 2H), 2.24-2.09 (m, 7H), 2.07-1.99 (m, 3H), 1.89-1.68 (m, 2H), 1.62- 1.33 (m, 1H); LCMS (Method D): t_{R} 3.23 min, 99%, MS (ESI) 365.2 (M + H)⁺ |

### Example 43: Synthesis of 1-(3-(4-((3-fluorophenyl)amino)-6-morpholinopyrimidin-2-yl)piperidin-1-yl)ethan-1-one (00327)

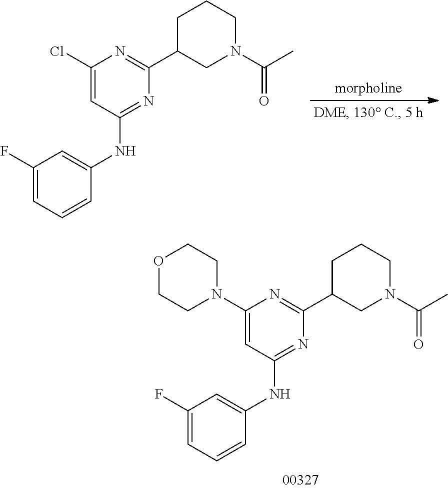 [SMILES: CC(=O)N1CCCC(c2nc(Cl)cc(Nc3cccc(F)c3)n2)C1 ; CC(=O)N1CCCC(c2nc(Nc3cccc(F)c3)cc(N3CCOCC3)n2)C1]

To a suspension of 1-(3-(4-chloro-6-((3-fluorophenyl)amino)pyrimidin-2-yl)piperidin-1-yl)ethan-1-one (30 mg, 0.086 mmol) in 1,2-dimethoxyethane (2 mL) in a 2 mL MW vial, morpholine (0.075 mL, 0.86 mmol) was added. The vial was capped and heated in a MW at 130° C. for 5 hours. The mixture was, cooled to room temperature, diluted with water (1 mL) and purified by using reversed phase chromatography (Method A) to afford 1-(3-(4-((3-fluorophenyl)amino)-6-morpholinopyrimidin-2-yl)piperidin-1-yl)ethan-1-one (25 mg, 73%) as a white solid. ¹H-NMR (400 MHz, DMSO-d6) mixture of rotamers δ 9.38-9.25 (m, 1H), 7.85-7.71 (m, 1H), 7.33-7.21 (m, 2H), 6.79-6.64 (m, 1H), 5.87-5.78 (m, 1H), 4.69-4.58 (m, 0.5H), 4.26-4.15 (m, 0.5H), 4.08-3.98 (m, 0.5H), 3.89-3.77 (m, 0.5H), 3.73-3.61 (m, 4H), 3.53-3.38 (m, 4.5H), 3.07-2.95 (m, 0.5H), 2.77-2.63 (m, 2H), 2.17-2.06 (m, 1H), 2.04-1.95 (m, 3H), 1.83-1.63 (m, 2H), 1.59-1.33 (m, 1H); LCMS (Method B): t_{R} 2.74 min, 97%, MS (ESI) 400.2 (M+H)⁺.

### Example 44: Synthesis of (+/−)-cis-1-(2-methyl-5-(4-((5-methylpyridin-3-yl)amino)-6-(1H-pyrazolo[3,4-c]pyridin-4-yl)pyrimidin-2-yl)piperidin-1-yl)ethan-1-one (00328)

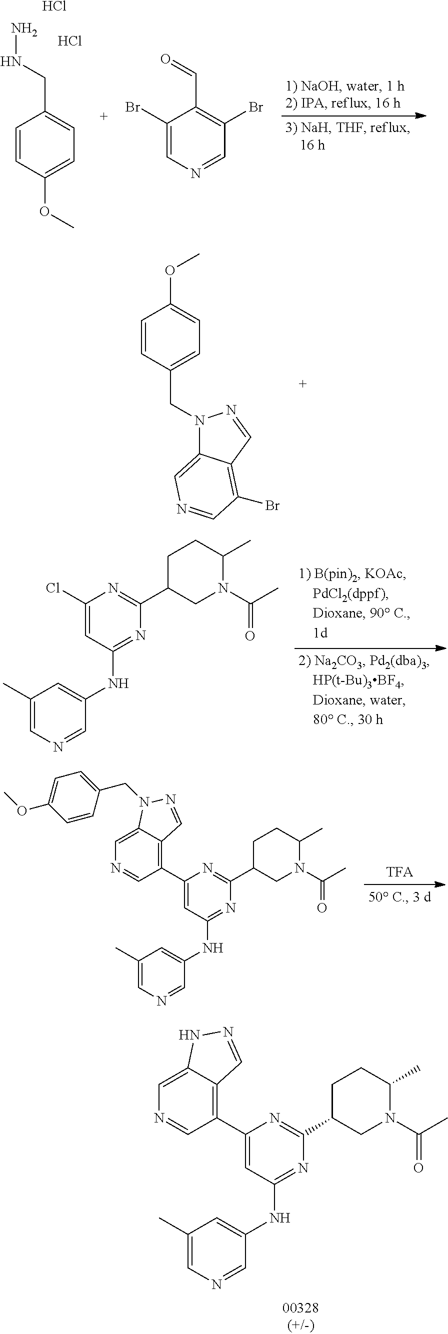

To a suspension of (4-methoxybenzyl)hydrazine dihydrochloride (4.3 g, 19 mmol) in methanol (50 mL), 50% sodium hydroxide in water (1.0 mL, 38 mmol) was added and the mixture was stirred at room temperature for 1 hour. The salts were filtrated off over a glass filter and washed with methanol. The filtrate was concentrated to afford a sticky white solid, which was suspended in 2-propanol (50 mL) and 3,5-dibromoisonicotinaldehyde (5.0 g, 19 mmol) was added. The mixture was stirred at reflux overnight resulting in an orangebrown suspension. This was allowed to cool to room temperature and water (25 mL) was added. The mixture was stirred at room temperature for 1 hour and the resulting precipitate was filtrated off and washed with 2-propanol/water (4/1, v/v, 50 mL). The solid was transferred to a flask and co-evaporated twice with ethyl acetate. The residue was suspended in dry tetrahydrofuran (100 mL) at room temperature and sodium hydride (0.38 g, 9.5 mmol) was added. The mixture was stirred for 10 minutes at room temperature and was then stirred at reflux overnight. The mixture was cooled to room temperature, poured onto water (300 mL) and extracted with ethyl acetate (2×150 mL). The combined organic layers were washed with brine, dried with sodium sulfate and concentrated to afford the crude product that was used as such in the next step. ¹H NMR (400 MHz, DMSO-d₆) δ 9.28 (s, 1H), 8.39 (s, 1H), 8.25 (s, 1H), 7.37-7.24 (m, 2H), 6.96-6.84 (m, 2H), 5.74 (s, 2H), 3.71 (s, 3H); LCMS (Method A): t_{R} 2.00 min, 92%, MS (ESI) 318.0/320.0 (M+H)⁺. A nitrogen flushed mixture of 4-bromo-1-(4-methoxybenzyl)-1H-pyrazolo[3,4-c]pyridine (177 mg, 0.56 mmol), bis(pinacolato)diboron (155 mg, 0.61 mmol), potassium acetate (82 mg, 0.83 mmol) and 1,1′-bis(diphenylphosphino)ferrocene-palladium(II) dichloride (23 mg, 0.028 mmol) in 1,4-dioxane (3 mL) was stirred at 80° C. for 2 hours. Additional bis(pinacolato)diboron (155 mg, 0.61 mmol), potassium acetate (82 mg, 0.83 mmol) and 1,1′-bis(diphenylphosphino)ferrocene-palladium(II) dichloride (23 mg, 0.028 mmol) were added and the reaction was stirred at 90° C. for 16 hours. The mixture was cooled to room temperature and 1-(5-(4-chloro-6-((5-methylpyridin-3-yl)amino)pyrimidin-2-yl)-2-methylpiperidin-1-yl)ethan-1-one (100 mg, 0.28 mmol), sodium carbonate (59 mg, 0.56 mmol), tri-tert-butylphosphonium tetrafluoroborate (8.1 mg, 30 μmol), tris(dibenzylideneacetone)dipalladium(0) (13 mg, 10 μmol), 1,4-dioxane (3 mL) and water (1 mL) were added. The mixture was stirred at 80° C. for 30 hours. The reaction mixture was allowed to cool to room temperature and stirred overnight. Solids were removed by filtration and the reaction mixture was filtered over a small C₁₈-plug using acetonitrile as eluent. The product was purified by using reversed phase chromatography (Method A) followed by a second purification using reversed phase chromatography (Method B) to afford 1-(5-(4-(1-(4-methoxybenzyl)-1H-pyrazolo[3,4-c]pyridin-4-yl)-6-((5-methylpyridin-3-yl)amino)pyrimidin-2-yl)-2-methylpiperidin-1-yl)ethan-1-one (13 mg, 8%) as a light brown solid. ¹H NMR (400 MHz, DMSO-d₆) mixture of rotamers δ 9.91 (d, J=4.8 Hz, 1H), 9.39 (d, J=2.3 Hz, 1H), 8.83 (d, J=1.6 Hz, 1H), 8.76-8.63 (m, 2H), 8.19 (d, J=14.2 Hz, 1H), 8.10 (s, 1H), 7.36-7.27 (m, 3H), 6.92-6.86 (m, 2H), 5.79 (s, 2H), 4.91-4.77 (m, 1H), 4.30-4.16 (m, 0.5H), 4.14-4.02 (m, 0.5H), 3.71 (s, 3H), 3.52-3.39 (m, 0.5H), 3.04-2.71 (m, 1.5H), 2.33 (d, J=4.4 Hz, 3H), 2.15-1.98 (m, 5H), 1.90-1.66 (m, 2H), 1.28 (d, J=6.8 Hz, 1.5H), 1.16 (d, J=6.9 Hz, 1.5H); LCMS (Method C): t_{R} 2.01 min, 98%, MS (ESI) 563.2 (M+H)⁺. A solution of 1-(5-(4-(1-(4-methoxybenzyl)-1H-pyrazolo[3,4-c]pyridin-4-yl)-6-((5-methylpyridin-3-yl)amino)pyrimidin-2-yl)-2-methylpiperidin-1-yl)ethan-1-one (13 mg, 23 μmol) in TFA (1 mL) was stirred at room temperature for 3 hours and was then heated to 50° C. and stirred for 3 days. The reaction mixture was concentrated in vacuo and purified using reversed phase chromatography (Method B) to afford (+/−)-cis-1-(2-methyl-5-(4-((5-methylpyridin-3-yl)amino)-6-(1H-pyrazolo[3,4-c]pyridin-4-yl)pyrimidin-2-yl)piperidin-1-yl)ethan-1-one (9 mg, 88%) as a white solid. ¹H NMR (400 MHz, DMSO-d₆) mixture of rotamers δ 13.93 (s, 1H), 9.90 (d, J=4.6 Hz, 1H), 9.18 (s, 1H), 8.84 (s, 1H), 8.77-8.65 (m, 2H), 8.20 (d, J=14.8 Hz, 1H), 8.10 (s, 1H), 7.37 (d, J=1.7 Hz, 1H), 4.91-4.77 (m, 1H), 4.29-4.18 (m, 0.5H), 4.15-4.01 (m, 0.5H), 3.53-3.41 (m, 0.5H), 3.04-2.74 (m, 1.5H), 2.34 (d, J=4.5 Hz, 3H), 2.17-1.97 (m, 5H), 1.94-1.64 (m, 2H), 1.29 (d, J=6.8 Hz, 1.5H), 1.16 (d, J=7.0 Hz, 1.5H); LCMS (Method D): t_{R} 3.02 min, 97%, MS (ESI) 443.2 (M+H)⁺.

The following compounds were prepared using procedures analogous to Example 44.

| Compound # | Structure and compound name | Analytical data |
|---|---|---|
| 00329 | 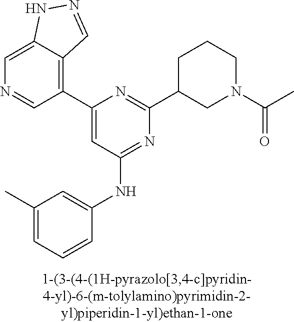 [SMILES: CC(=O)N1CCCC(c2nc(Nc3cccc(C)c3)cc(-c3cncc4[nH]ncc34)n2)C1] | ¹H-NMR (400 MHz, DMSO-d6) mixture of rotamers δ 9.72-9.62 (m, 1H), 9.13 (s, 1H), 8.82-8.74 (m, 1H), 8.67 (s, 1H), 7.72-7.50 (m, 3H), 7.38- 7.09 (m, 3H), 6.91-6.81 (m, 1H), 4.92-4.81 (m, 0.5H), 4.33-4.15 (m, 1H), 3.93-3.83 (m, 0.5H), 3.57-3.47 (m, 0.5H), 3.15-3.05 (m, 0.5H), 3.05- 2.95 (m, 0.5H), 2.92-2.72 (m, 1.5H), 2.37-2.22 (m, 4H), 2.06 (s, 3H), 2.01-1.71 (m, 2H), 1.69-1.41 (m, 1H); LCMS (Method D): t_{R} 3.43 min, 100%, MS (ESI) 428.2 (M + H)⁺ |

### Example 45: Synthesis of 1-(3-(4-(5-(4-acetylpiperazin-1-yl)pyridin-3-yl)-6-((3-fluorophenyl)amino)pyrimidin-2-yl)piperidin-1-yl)ethan-1-one (00331)

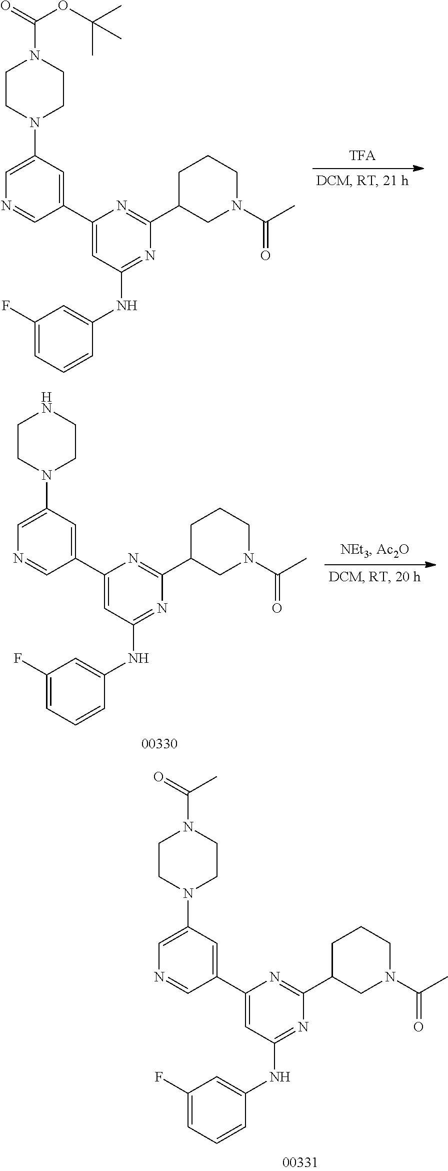

To a solution of tert-butyl 4-(5-(2-(1-acetylpiperidin-3-yl)-6-((3-fluorophenyl)amino)pyrimidin-4-yl)pyridin-3-yl)piperazine-1-carboxylate (79 mg, 0.14 mmol) in dichloromethane (2 mL) was added TFA (0.021 mL, 0.27 mmol) and the reaction mixture was stirred at room temperature for 20 hours. Additional TFA (0.1 mL, 1.31 mmol) was added and the reaction mixture was stirred for 1 hour, concentrated and purified using reversed phase chromatography (Method B) to afford 1-(3-(4-((3-fluorophenyl)amino)-6-(5-(piperazin-1-yl)pyridin-3-yl)pyrimidin-2-yl)piperidin-1-yl)ethan-1-one (57 mg, 87%) as a white solid. ¹H NMR (400 MHz, chloroform-d) mixture of rotamers δ 8.59-8.50 (m, 1H), 8.42-8.34 (m, 1H), 7.92-7.82 (m, 1H), 7.49-7.29 (m, 2H), 7.19-7.10 (m, 1H), 6.97-6.93 (m, 1H), 6.91-6.83 (m, 2H), 4.98-4.90 (m, 0.5H), 4.55-4.44 (m, 0.5H), 4.17-4.07 (m, 0.5H), 3.92-3.82 (m, 0.5H), 3.62-3.52 (m, 0.5H), 3.33-3.24 (m, 4H), 3.20-2.93 (m, 6H), 2.87-2.78 (m, 0.5H), 2.34-2.25 (m, 1H), 2.16 (s, 3H), 2.01-1.83 (m, 2H), 1.73-1.60 (m, 1H); LCMS (Method D): t_{R} 3.43 min, 99%, MS (ESI) 476.2 (M+H)⁺. To a solution of 1-(3-(4-((3-fluorophenyl)amino)-6-(5-(piperazin-1-yl)pyridin-3-yl)pyrimidin-2-yl)piperidin-1-yl)ethan-1-one (21 mg, 0.044 mmol) in dichloromethane (1 mL) was added acetic anhydride (5.4 μl, 0.057 mmol) and triethylamine (0.012 mL, 0.088 mmol) and the reaction was stirred at room temperature for 20 hours. The reaction mixture was concentrated and purified using reversed phase chromatography (Method B) to afford 1-(3-(4-(5-(4-acetylpiperazin-1-yl)pyridin-3-yl)-6-((3-fluorophenyl)amino)pyrimidin-2-yl)piperidin-1-yl)ethan-1-one (10 mg, 44%) as a white solid. ¹H NMR (400 MHz, DMSO-d₆) mixture of rotamers δ 10.05-9.91 (m, 1H), 8.66-8.58 (m, 1H), 8.50-8.42 (m, 1H), 7.96-7.80 (m, 2H), 7.46-7.31 (m, 2H), 7.20-7.11 (m, 1H), 6.89-6.79 (m, 1H), 4.74-4.64 (m, 0.5H), 4.24-4.06 (m, 1H), 3.89-3.79 (m, 0.5H), 3.68-3.58 (m, 4H), 3.58-3.48 (m, 0.5H), 3.30-3.25 (m, 4H), 3.15-3.05 (m, 0.5H), 3.02-2.90 (m, 1H), 2.87-2.74 (m, 1H), 2.29-2.16 (m, 1H), 2.06 (s, 3H), 2.04 (s, 3H), 1.95-1.70 (m, 2H), 1.68-1.40 (m, 1H); LCMS (Method D): t_{R} 3.36 min, 98%, MS (ESI) 518.2 (M+H)⁺.

Assay Data

One thousand four hundred A375 cells were seeded in each well of a 384 well microplate. One day later they were treated with 1 μM S1152 (Selleckchem) in dimethyl sulfoxide, 0.5 μM S1008 (Selleckchem) and dose ranges of invention compounds for one day in Dulbecco's Modified Eagle's Medium supplemented with 10% fetal calf serum and 2 mM (2S)-2-amino-4-carbamoylbutanoic acid. Wells were incubated with 4% polyoxymethylene (Sigma Aldrich 158127) at ambient temperature. After ten minutes, polyoxymethylene was replaced with phosphate-buffered saline. After one minute phosphate-buffered saline was replaced with 0.2% polyethylene glycol octylphenyl ether in phosphate-buffered saline. After ten minutes at ambient temperature 0.2% polyethylene glycol octylphenyl ether in phosphate-buffered saline was replaced with 1% aminoethanoic acid in phosphate-buffered saline. After ten minutes at ambient temperature 1% aminoethanoic acid in phosphate-buffered saline was replaced with 0.8 μg/ml sc-365823 (clone E-4, Santa Cruz Biotechnologies) in 0.05% Polyoxyethylene (20) sorbitan monolaurate in phosphate-buffered saline. After one hour at 37° C., the solution was replaced with phosphate-buffered saline twice. Phosphate-buffered saline was replaced with 2 μg/ml A-11029 (Invitrogen) in 0.05% polyoxyethylene (20) sorbitan monolaurate in phosphate-buffered saline. After one hour at ambient temperature in the dark, 12 μM 3,8-Diamino-5-[3-(diethylmethylammonio)propyl]-6-phenylphenanthridinium diiodide in phosphate-buffered saline containing 0.2 mg/ml ribonuclease A was added. After one hour at ambient temperature in the dark, signal in wells was measured with a Acumen Cellista. 3,8-Diamino-5-[3-(diethylmethylammonio)propyl]-6-phenylphenanthridinium diiodide-positive objects were defined in FL3 (565-600 nm) with a perimeter between 50-800 μm and a total intensity of 10000-1000000 FLU. Signal in FL2 (500-530 nm) was measured in defined objects. A normalized ratio of total ln FL2 over total ln FL3 staining was log transformed. A non-linear regression (variable slope, 4 parameters) was used to calculate EC₅₀ values using GraphPad Prism (Version 7.0d).

| Compound # | EC50 |
|---|---|
| 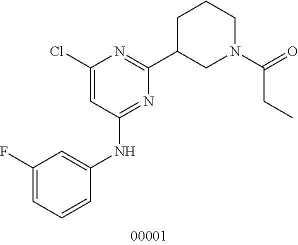 [SMILES: CCC(=O)N1CCCC(c2nc(Cl)cc(Nc3cccc(F)c3)n2)C1] | C |
| 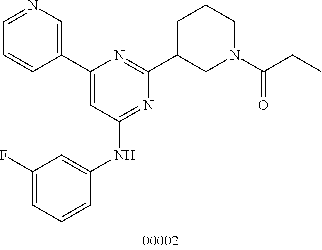 [SMILES: CCC(=O)N1CCCC(c2nc(Nc3cccc(F)c3)cc(-c3cccnc3)n2)C1] | B |
| 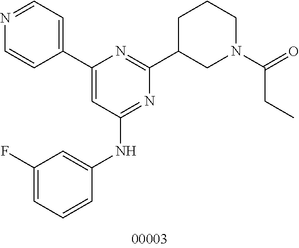 [SMILES: CCC(=O)N1CCCC(c2nc(Nc3cccc(F)c3)cc(-c3ccncc3)n2)C1] | C |
| 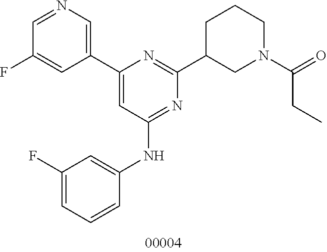 [SMILES: CCC(=O)N1CCCC(c2nc(Nc3cccc(F)c3)cc(-c3cncc(F)c3)n2)C1] | C |
| 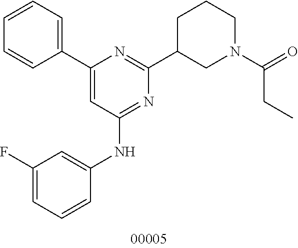 [SMILES: CCC(=O)N1CCCC(c2nc(Nc3cccc(F)c3)cc(-c3ccccc3)n2)C1] | B |
| 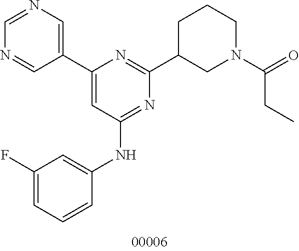 [SMILES: CCC(=O)N1CCCC(c2nc(Nc3cccc(F)c3)cc(-c3cncnc3)n2)C1] | B |
| 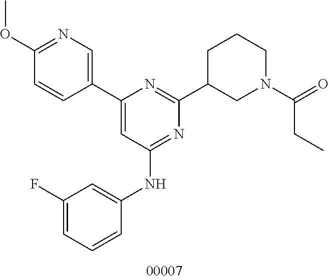 [SMILES: CCC(=O)N1CCCC(c2nc(Nc3cccc(F)c3)cc(-c3ccc(OC)nc3)n2)C1] | C |
| 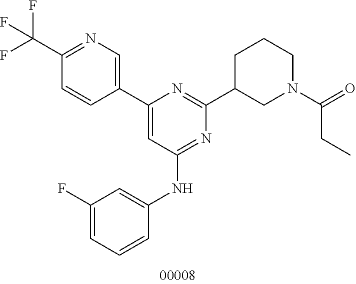 [SMILES: CCC(=O)N1CCCC(c2nc(Nc3cccc(F)c3)cc(-c3ccc(C(F)(F)F)nc3)n2)C1] | B |
| 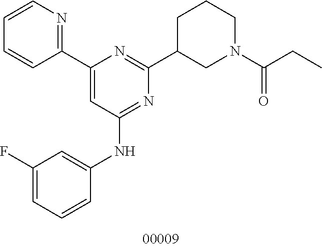 [SMILES: CCC(=O)N1CCCC(c2nc(Nc3cccc(F)c3)cc(-c3ccccn3)n2)C1] | C |
| 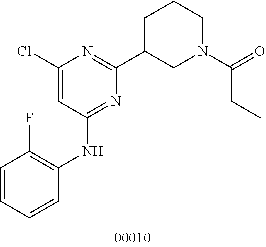 [SMILES: CCC(=O)N1CCCC(c2nc(Cl)cc(Nc3ccccc3F)n2)C1] | C |
| 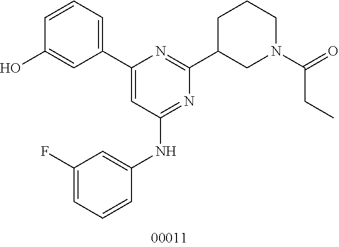 [SMILES: CCC(=O)N1CCCC(c2nc(Nc3cccc(F)c3)cc(-c3cccc(O)c3)n2)C1] | B |
| 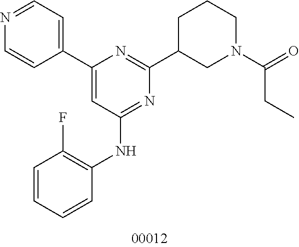 [SMILES: CCC(=O)N1CCCC(c2nc(Nc3ccccc3F)cc(-c3ccncc3)n2)C1] | C |
| 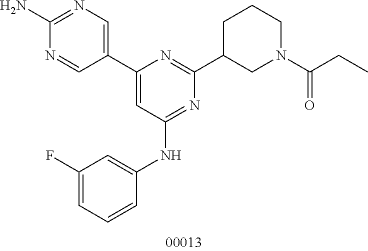 [SMILES: CCC(=O)N1CCCC(c2nc(Nc3cccc(F)c3)cc(-c3cnc(N)nc3)n2)C1] | C |
| 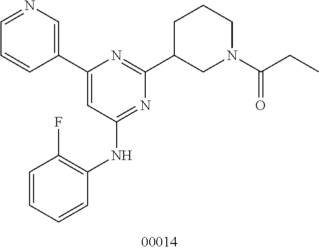 [SMILES: CCC(=O)N1CCCC(c2nc(Nc3ccccc3F)cc(-c3cccnc3)n2)C1] | B |
| 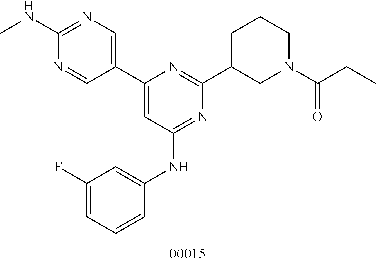 [SMILES: CCC(=O)N1CCCC(c2nc(Nc3cccc(F)c3)cc(-c3cnc(NC)nc3)n2)C1] | C |
| 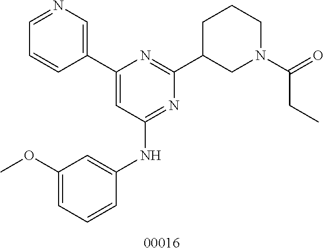 [SMILES: CCC(=O)N1CCCC(c2nc(Nc3cccc(OC)c3)cc(-c3cccnc3)n2)C1] | B |
| 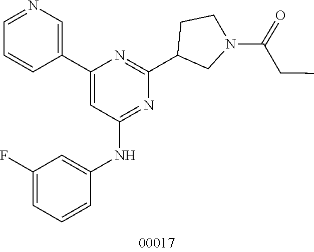 [SMILES: CCC(=O)N1CCC(c2nc(Nc3cccc(F)c3)cc(-c3cccnc3)n2)C1] | C |
| 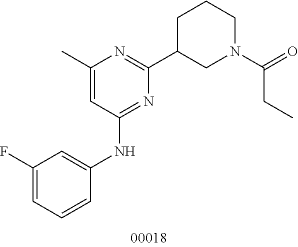 [SMILES: CCC(=O)N1CCCC(c2nc(C)cc(Nc3cccc(F)c3)n2)C1] | C |
| 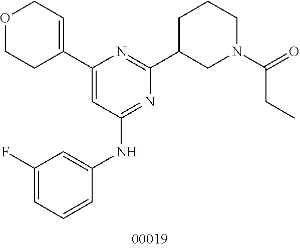 [SMILES: CCC(=O)N1CCCC(c2nc(Nc3cccc(F)c3)cc(C3=CCOCC3)n2)C1] | C |
| 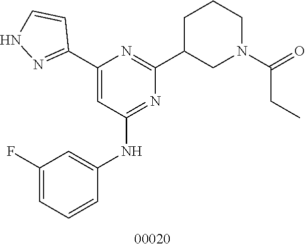 [SMILES: CCC(=O)N1CCCC(c2nc(Nc3cccc(F)c3)cc(-c3cc[nH]n3)n2)C1] | C |
| 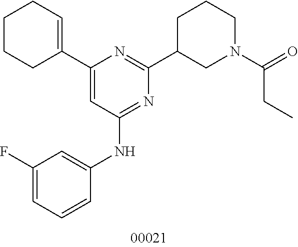 [SMILES: CCC(=O)N1CCCC(c2nc(Nc3cccc(F)c3)cc(C3=CCCCC3)n2)C1] | C |
| 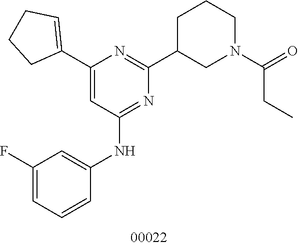 [SMILES: CCC(=O)N1CCCC(c2nc(Nc3cccc(F)c3)cc(C3=CCCC3)n2)C1] | C |
| 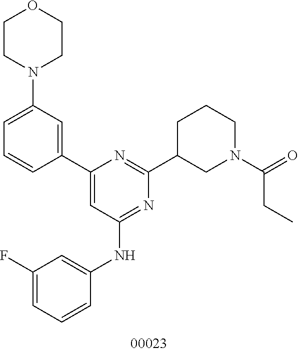 [SMILES: CCC(=O)N1CCCC(c2nc(Nc3cccc(F)c3)cc(-c3cccc(N4CCOCC4)c3)n2)C1] | B |
| 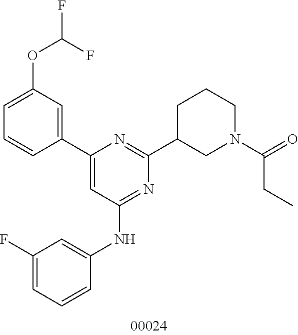 [SMILES: CCC(=O)N1CCCC(c2nc(Nc3cccc(F)c3)cc(-c3cccc(OC(F)F)c3)n2)C1] | C |
| 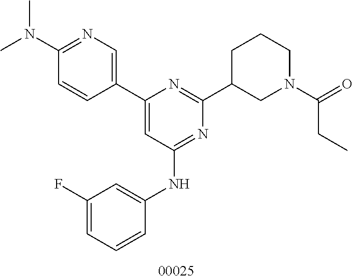 [SMILES: CCC(=O)N1CCCC(c2nc(Nc3cccc(F)c3)cc(-c3ccc(N(C)C)nc3)n2)C1] | C |
| 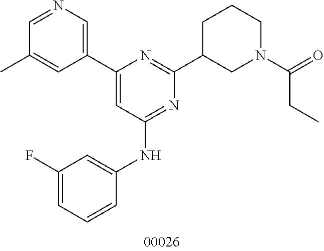 [SMILES: CCC(=O)N1CCCC(c2nc(Nc3cccc(F)c3)cc(-c3cncc(C)c3)n2)C1] | B |
| 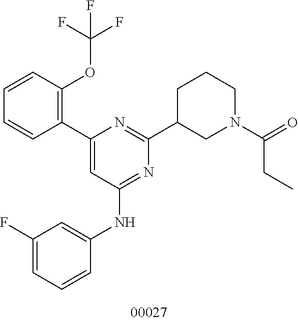 [SMILES: CCC(=O)N1CCCC(c2nc(Nc3cccc(F)c3)cc(-c3ccccc3OC(F)(F)F)n2)C1] | C |
| 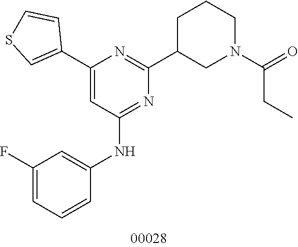 [SMILES: CCC(=O)N1CCCC(c2nc(Nc3cccc(F)c3)cc(-c3ccsc3)n2)C1] | C |
| 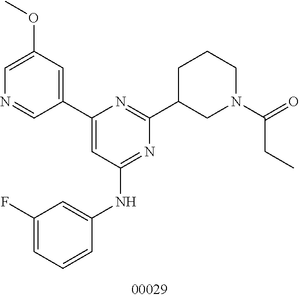 [SMILES: CCC(=O)N1CCCC(c2nc(Nc3cccc(F)c3)cc(-c3cncc(OC)c3)n2)C1] | B |
| 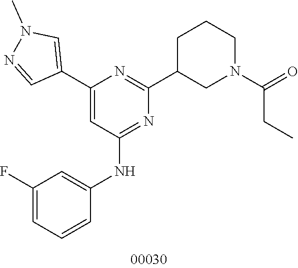 [SMILES: CCC(=O)N1CCCC(c2nc(Nc3cccc(F)c3)cc(-c3cnn(C)c3)n2)C1] | C |
| 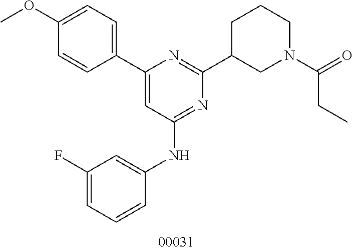 [SMILES: CCC(=O)N1CCCC(c2nc(Nc3cccc(F)c3)cc(-c3ccc(OC)cc3)n2)C1] | B |
| 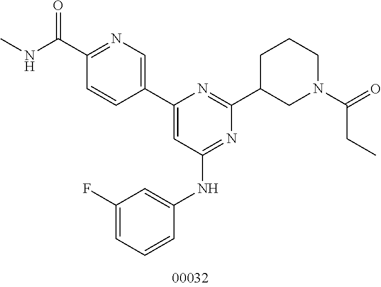 [SMILES: CCC(=O)N1CCCC(c2nc(Nc3cccc(F)c3)cc(-c3ccc(C(=O)NC)nc3)n2)C1] | C |
| 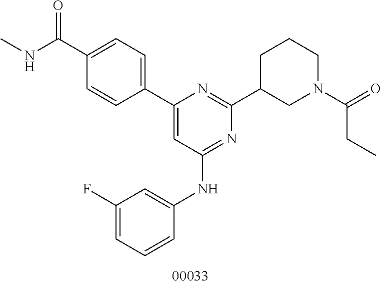 [SMILES: CCC(=O)N1CCCC(c2nc(Nc3cccc(F)c3)cc(-c3ccc(C(=O)NC)cc3)n2)C1] | B |
| 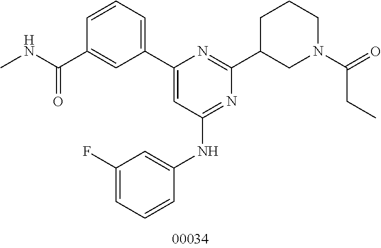 [SMILES: CCC(=O)N1CCCC(c2nc(Nc3cccc(F)c3)cc(-c3cccc(C(=O)NC)c3)n2)C1] | C |
| 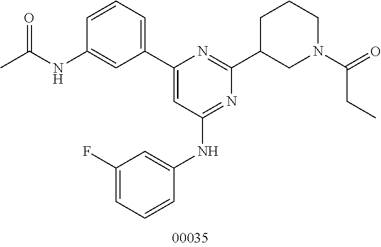 [SMILES: CCC(=O)N1CCCC(c2nc(Nc3cccc(F)c3)cc(-c3cccc(NC(C)=O)c3)n2)C1] | B |
| 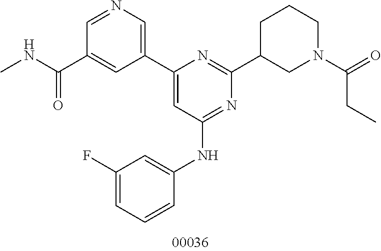 [SMILES: CCC(=O)N1CCCC(c2nc(Nc3cccc(F)c3)cc(-c3cncc(C(=O)NC)c3)n2)C1] | B |
| 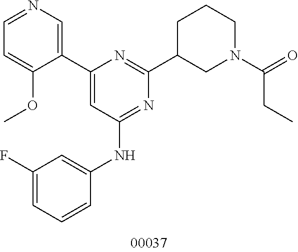 [SMILES: CCC(=O)N1CCCC(c2nc(Nc3cccc(F)c3)cc(-c3cnccc3OC)n2)C1] | B |
| 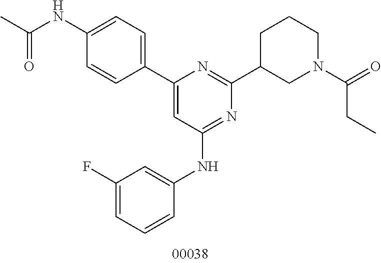 [SMILES: CCC(=O)N1CCCC(c2nc(Nc3cccc(F)c3)cc(-c3ccc(NC(C)=O)cc3)n2)C1] | B |
| 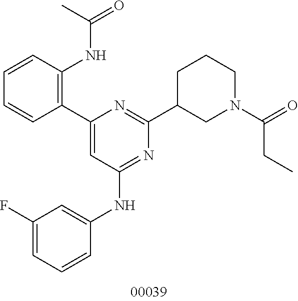 [SMILES: CCC(=O)N1CCCC(c2nc(Nc3cccc(F)c3)cc(-c3ccccc3NC(C)=O)n2)C1] | C |
| 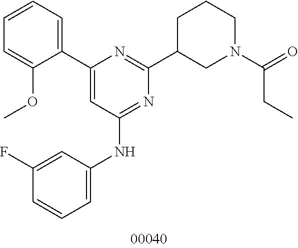 [SMILES: CCC(=O)N1CCCC(c2nc(Nc3cccc(F)c3)cc(-c3ccccc3OC)n2)C1] | C |
| 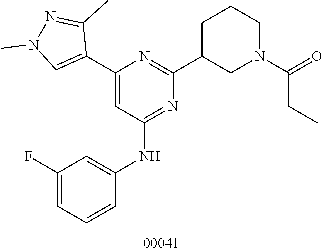 [SMILES: CCC(=O)N1CCCC(c2nc(Nc3cccc(F)c3)cc(-c3cn(C)nc3C)n2)C1] | C |
| 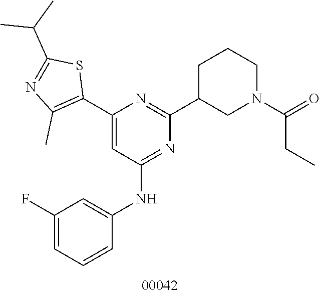 [SMILES: CCC(=O)N1CCCC(c2nc(Nc3cccc(F)c3)cc(-c3sc(C(C)C)nc3C)n2)C1] | B |
| 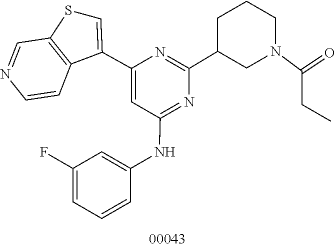 [SMILES: CCC(=O)N1CCCC(c2nc(Nc3cccc(F)c3)cc(-c3csc4cnccc34)n2)C1] | B |
| 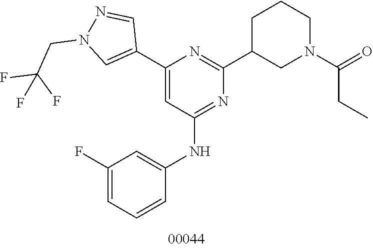 [SMILES: CCC(=O)N1CCCC(c2nc(Nc3cccc(F)c3)cc(-c3cnn(CC(F)(F)F)c3)n2)C1] | B |
| 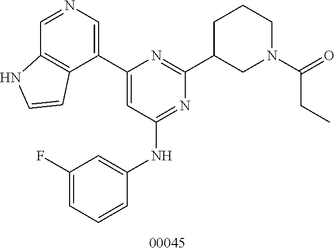 [SMILES: CCC(=O)N1CCCC(c2nc(Nc3cccc(F)c3)cc(-c3cncc4[nH]ccc34)n2)C1] | C |
| 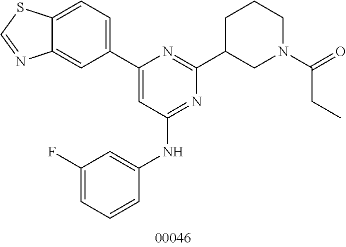 [SMILES: CCC(=O)N1CCCC(c2nc(Nc3cccc(F)c3)cc(-c3ccc4scnc4c3)n2)C1] | B |
| 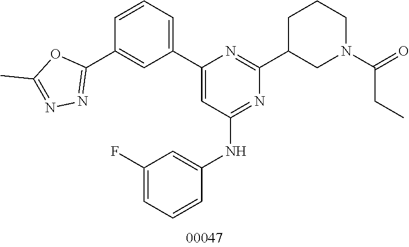 [SMILES: CCC(=O)N1CCCC(c2nc(Nc3cccc(F)c3)cc(-c3cccc(-c4nnc(C)o4)c3)n2)C1] | B |
| 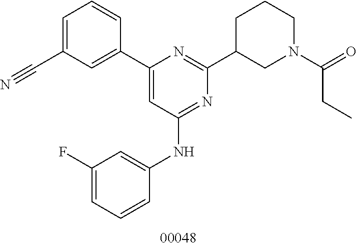 [SMILES: CCC(=O)N1CCCC(c2nc(Nc3cccc(F)c3)cc(-c3cccc(C#N)c3)n2)C1] | B |
| 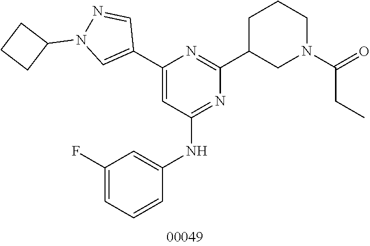 [SMILES: CCC(=O)N1CCCC(c2nc(Nc3cccc(F)c3)cc(-c3cnn(C4CCC4)c3)n2)C1] | C |
| 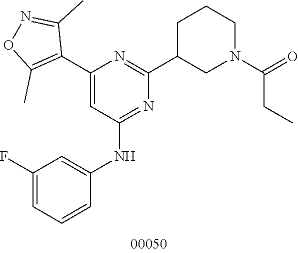 [SMILES: CCC(=O)N1CCCC(c2nc(Nc3cccc(F)c3)cc(-c3c(C)noc3C)n2)C1] | B |
| 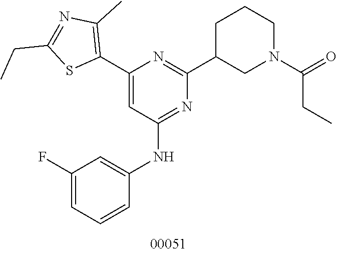 [SMILES: CCC(=O)N1CCCC(c2nc(Nc3cccc(F)c3)cc(-c3sc(CC)nc3C)n2)C1] | C |
| 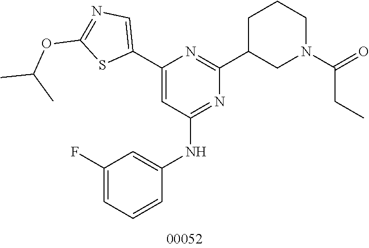 [SMILES: CCC(=O)N1CCCC(c2nc(Nc3cccc(F)c3)cc(-c3cnc(OC(C)C)s3)n2)C1] | C |
| 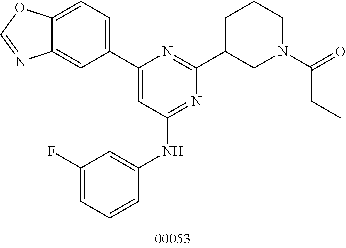 [SMILES: CCC(=O)N1CCCC(c2nc(Nc3cccc(F)c3)cc(-c3ccc4ocnc4c3)n2)C1] | C |
| 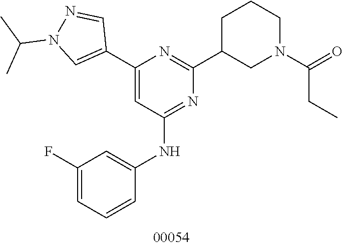 [SMILES: CCC(=O)N1CCCC(c2nc(Nc3cccc(F)c3)cc(-c3cnn(C(C)C)c3)n2)C1] | C |
| 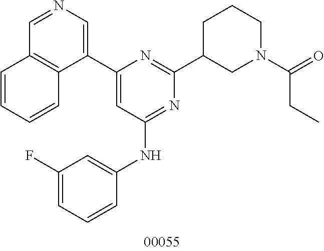 [SMILES: CCC(=O)N1CCCC(c2nc(Nc3cccc(F)c3)cc(-c3cncc4ccccc34)n2)C1] | C |
| 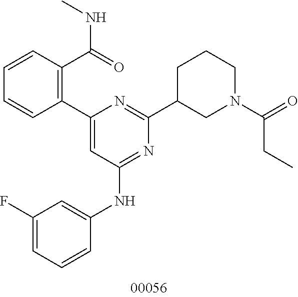 [SMILES: CCC(=O)N1CCCC(c2nc(Nc3cccc(F)c3)cc(-c3ccccc3C(=O)NC)n2)C1] | C |
| 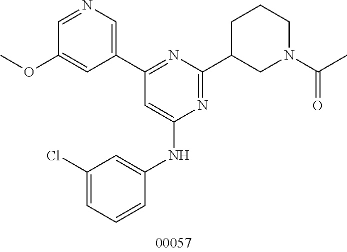 [SMILES: COc1cncc(-c2cc(Nc3cccc(Cl)c3)nc(C3CCCN(C(C)=O)C3)n2)c1] | A |
| 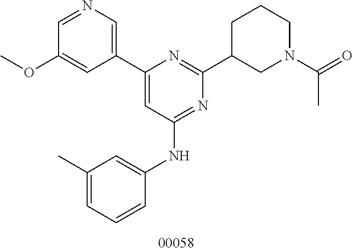 [SMILES: COc1cncc(-c2cc(Nc3cccc(C)c3)nc(C3CCCN(C(C)=O)C3)n2)c1] | A |
| 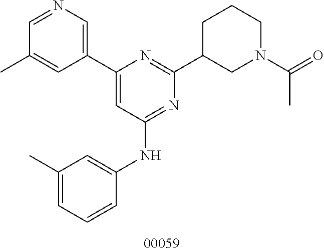 [SMILES: CC(=O)N1CCCC(c2nc(Nc3cccc(C)c3)cc(-c3cncc(C)c3)n2)C1] | A |
| 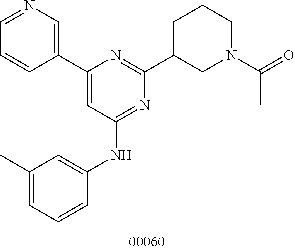 [SMILES: CC(=O)N1CCCC(c2nc(Nc3cccc(C)c3)cc(-c3cccnc3)n2)C1] | A |
| 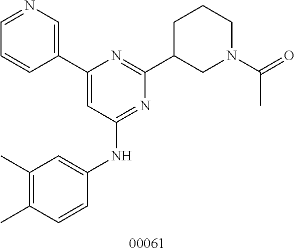 [SMILES: CC(=O)N1CCCC(c2nc(Nc3ccc(C)c(C)c3)cc(-c3cccnc3)n2)C1] | A |
| 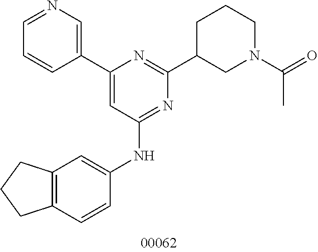 [SMILES: CC(=O)N1CCCC(c2nc(Nc3ccc4c(c3)CCC4)cc(-c3cccnc3)n2)C1] | C |
| 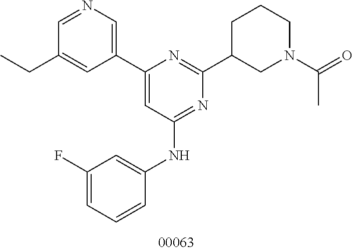 [SMILES: CCc1cncc(-c2cc(Nc3cccc(F)c3)nc(C3CCCN(C(C)=O)C3)n2)c1] | A |
| 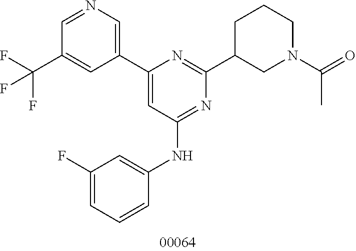 [SMILES: CC(=O)N1CCCC(c2nc(Nc3cccc(F)c3)cc(-c3cncc(C(F)(F)F)c3)n2)C1] | C |
| 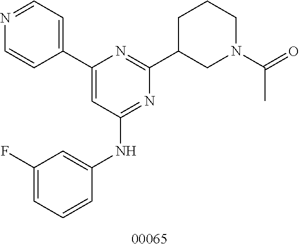 [SMILES: CC(=O)N1CCCC(c2nc(Nc3cccc(F)c3)cc(-c3ccncc3)n2)C1] | B |
| 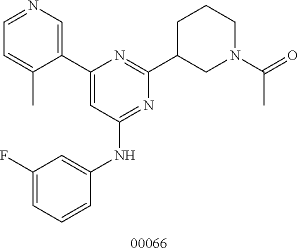 [SMILES: CC(=O)N1CCCC(c2nc(Nc3cccc(F)c3)cc(-c3cnccc3C)n2)C1] | B |
| 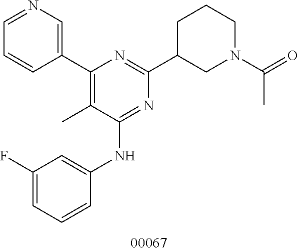 [SMILES: CC(=O)N1CCCC(c2nc(Nc3cccc(F)c3)c(C)c(-c3cccnc3)n2)C1] | C |
| 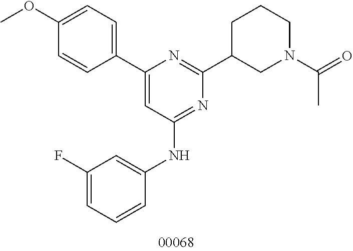 [SMILES: COc1ccc(-c2cc(Nc3cccc(F)c3)nc(C3CCCN(C(C)=O)C3)n2)cc1] | B |
| 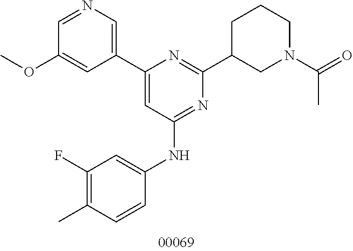 [SMILES: COc1cncc(-c2cc(Nc3ccc(C)c(F)c3)nc(C3CCCN(C(C)=O)C3)n2)c1] | B |
| 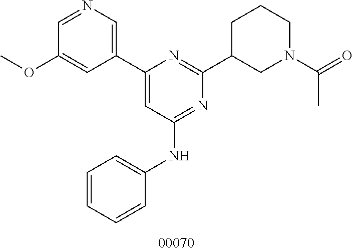 [SMILES: COc1cncc(-c2cc(Nc3ccccc3)nc(C3CCCN(C(C)=O)C3)n2)c1] | A |
| 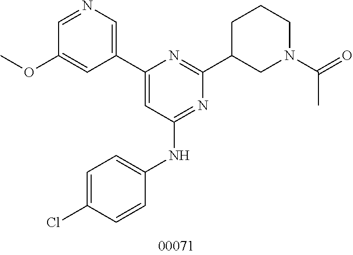 [SMILES: COc1cncc(-c2cc(Nc3ccc(Cl)cc3)nc(C3CCCN(C(C)=O)C3)n2)c1] | B |
| 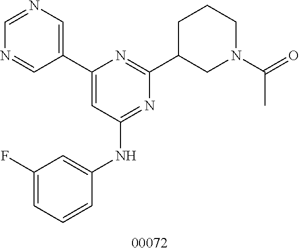 [SMILES: CC(=O)N1CCCC(c2nc(Nc3cccc(F)c3)cc(-c3cncnc3)n2)C1] | B |
| 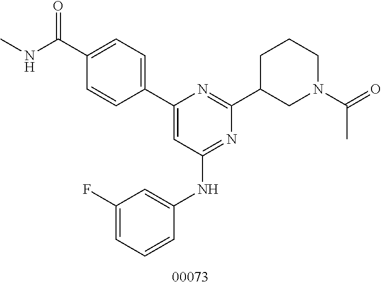 [SMILES: CNC(=O)c1ccc(-c2cc(Nc3cccc(F)c3)nc(C3CCCN(C(C)=O)C3)n2)cc1] | B |
| 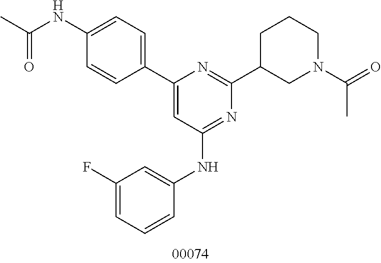 [SMILES: CC(=O)Nc1ccc(-c2cc(Nc3cccc(F)c3)nc(C3CCCN(C(C)=O)C3)n2)cc1] | A |
| 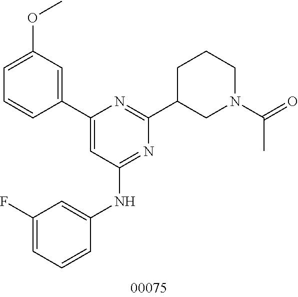 [SMILES: COc1cccc(-c2cc(Nc3cccc(F)c3)nc(C3CCCN(C(C)=O)C3)n2)c1] | B |
| 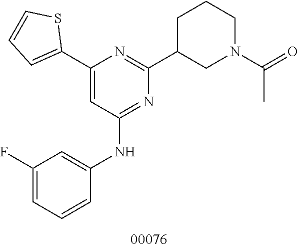 [SMILES: CC(=O)N1CCCC(c2nc(Nc3cccc(F)c3)cc(-c3cccs3)n2)C1] | B |
| 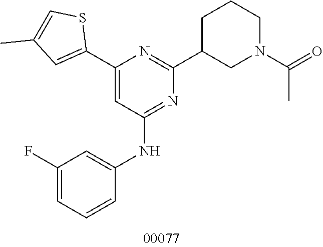 [SMILES: CC(=O)N1CCCC(c2nc(Nc3cccc(F)c3)cc(-c3cc(C)cs3)n2)C1] | A |
| 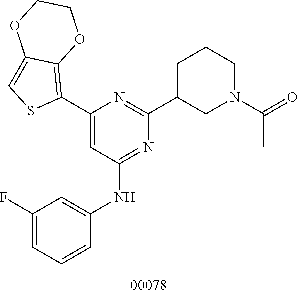 [SMILES: CC(=O)N1CCCC(c2nc(Nc3cccc(F)c3)cc(-c3scc4c3OCCO4)n2)C1] | A |
| 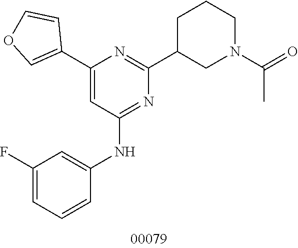 [SMILES: CC(=O)N1CCCC(c2nc(Nc3cccc(F)c3)cc(-c3ccoc3)n2)C1] | B |
| 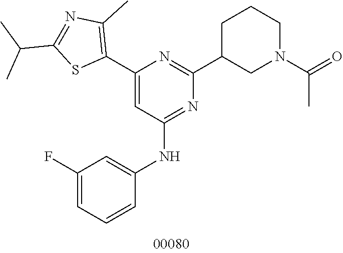 [SMILES: CC(=O)N1CCCC(c2nc(Nc3cccc(F)c3)cc(-c3sc(C(C)C)nc3C)n2)C1] | B |
| 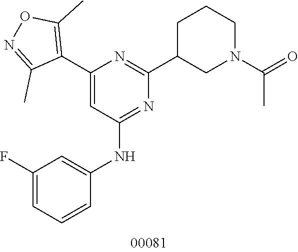 [SMILES: CC(=O)N1CCCC(c2nc(Nc3cccc(F)c3)cc(-c3c(C)noc3C)n2)C1] | B |
| 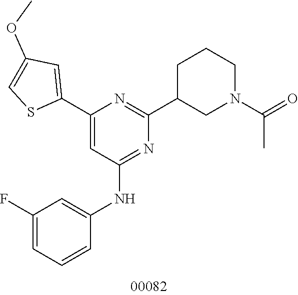 [SMILES: COc1csc(-c2cc(Nc3cccc(F)c3)nc(C3CCCN(C(C)=O)C3)n2)c1] | A |
| 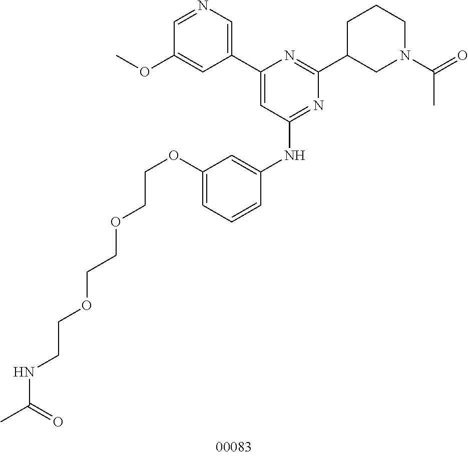 [SMILES: COc1cncc(-c2cc(Nc3cccc(OCCOCCOCCNC(C)=O)c3)nc(C3CCCN(C(C)=O)C3)n2)c1] | B |
| 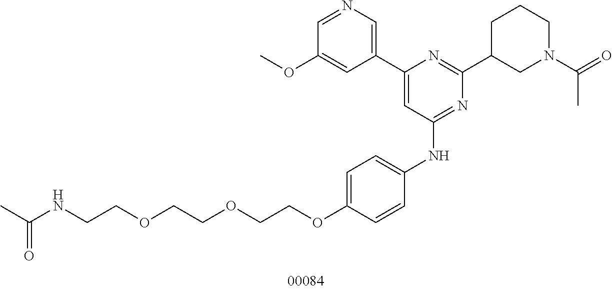 [SMILES: COc1cncc(-c2cc(Nc3ccc(OCCOCCOCCNC(C)=O)cc3)nc(C3CCCN(C(C)=O)C3)n2)c1] | C |
| 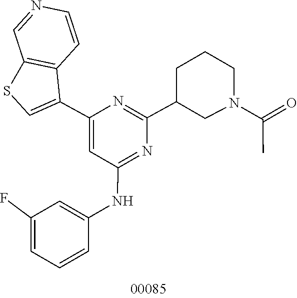 [SMILES: CC(=O)N1CCCC(c2nc(Nc3cccc(F)c3)cc(-c3csc4cnccc34)n2)C1] | C |
| 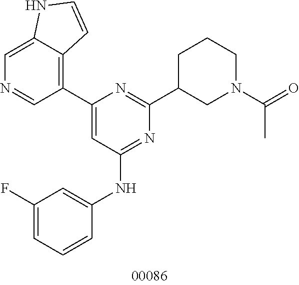 [SMILES: CC(=O)N1CCCC(c2nc(Nc3cccc(F)c3)cc(-c3cncc4[nH]ccc34)n2)C1] | A |
| 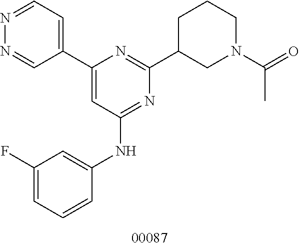 [SMILES: CC(=O)N1CCCC(c2nc(Nc3cccc(F)c3)cc(-c3ccnnc3)n2)C1] | B |
| 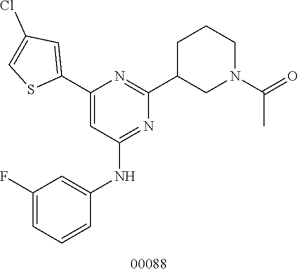 [SMILES: CC(=O)N1CCCC(c2nc(Nc3cccc(F)c3)cc(-c3cc(Cl)cs3)n2)C1] | B |
| 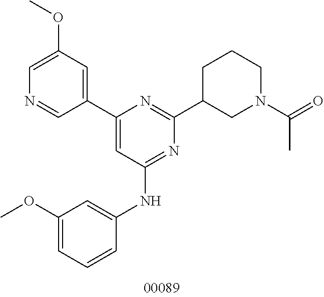 [SMILES: COc1cccc(Nc2cc(-c3cncc(OC)c3)nc(C3CCCN(C(C)=O)C3)n2)c1] | B |
| 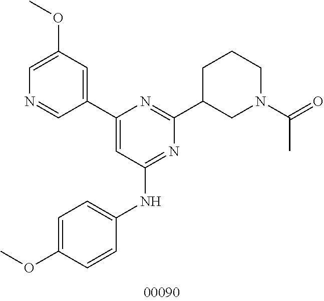 [SMILES: COc1ccc(Nc2cc(-c3cncc(OC)c3)nc(C3CCCN(C(C)=O)C3)n2)cc1] | B |
| 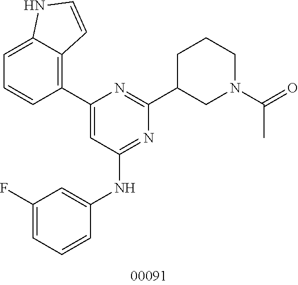 [SMILES: CC(=O)N1CCCC(c2nc(Nc3cccc(F)c3)cc(-c3cccc4[nH]ccc34)n2)C1] | A |
| 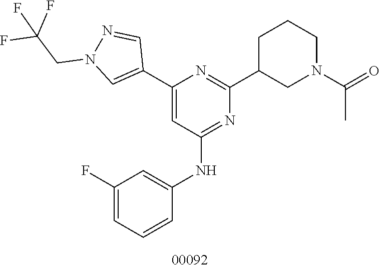 [SMILES: CC(=O)N1CCCC(c2nc(Nc3cccc(F)c3)cc(-c3cnn(CC(F)(F)F)c3)n2)C1] | B |
| 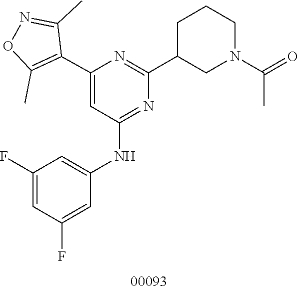 [SMILES: CC(=O)N1CCCC(c2nc(Nc3cc(F)cc(F)c3)cc(-c3c(C)noc3C)n2)C1] | B |
| 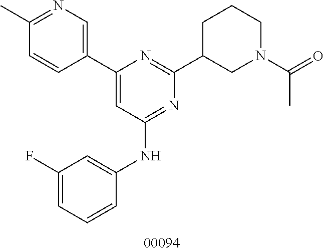 [SMILES: CC(=O)N1CCCC(c2nc(Nc3cccc(F)c3)cc(-c3ccc(C)nc3)n2)C1] | B |
| 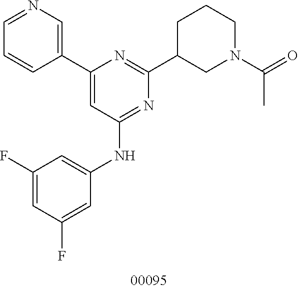 [SMILES: CC(=O)N1CCCC(c2nc(Nc3cc(F)cc(F)c3)cc(-c3cccnc3)n2)C1] | A |
| 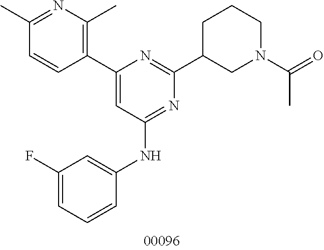 [SMILES: CC(=O)N1CCCC(c2nc(Nc3cccc(F)c3)cc(-c3ccc(C)nc3C)n2)C1] | B |
| 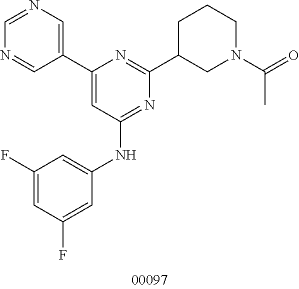 [SMILES: CC(=O)N1CCCC(c2nc(Nc3cc(F)cc(F)c3)cc(-c3cncnc3)n2)C1] | B |
| 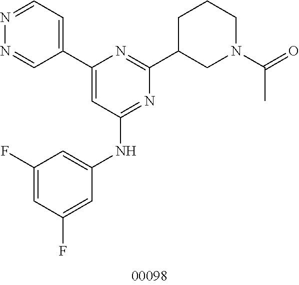 [SMILES: CC(=O)N1CCCC(c2nc(Nc3cc(F)cc(F)c3)cc(-c3ccnnc3)n2)C1] | B |
| 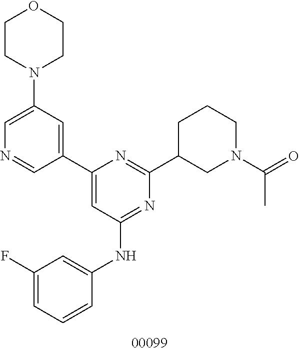 [SMILES: CC(=O)N1CCCC(c2nc(Nc3cccc(F)c3)cc(-c3cncc(N4CCOCC4)c3)n2)C1] | A |
| 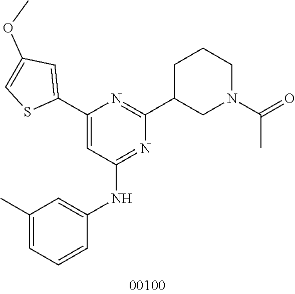 [SMILES: COc1csc(-c2cc(Nc3cccc(C)c3)nc(C3CCCN(C(C)=O)C3)n2)c1] | A |
| 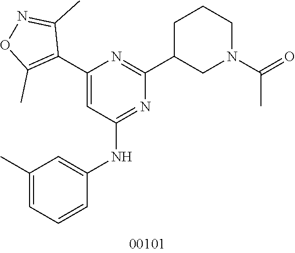 [SMILES: CC(=O)N1CCCC(c2nc(Nc3cccc(C)c3)cc(-c3c(C)noc3C)n2)C1] | B |
| 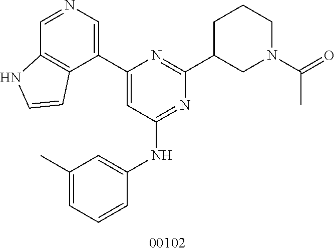 [SMILES: CC(=O)N1CCCC(c2nc(Nc3cccc(C)c3)cc(-c3cncc4[nH]ccc34)n2)C1] | A |
| 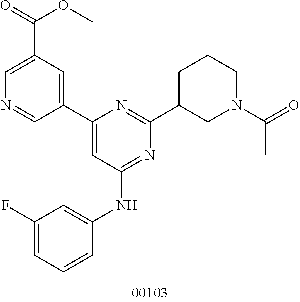 [SMILES: COC(=O)c1cncc(-c2cc(Nc3cccc(F)c3)nc(C3CCCN(C(C)=O)C3)n2)c1] | C |
| 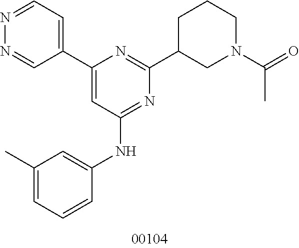 [SMILES: CC(=O)N1CCCC(c2nc(Nc3cccc(C)c3)cc(-c3ccnnc3)n2)C1] | B |
| 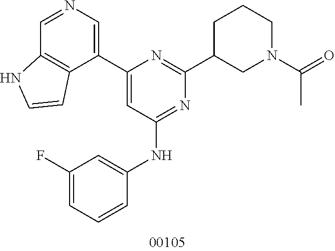 [SMILES: CC(=O)N1CCCC(c2nc(Nc3cccc(F)c3)cc(-c3cncc4[nH]ccc34)n2)C1] | B |
| 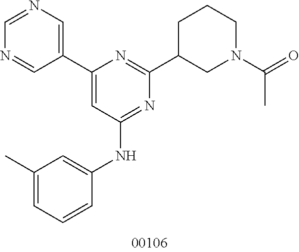 [SMILES: CC(=O)N1CCCC(c2nc(Nc3cccc(C)c3)cc(-c3cncnc3)n2)C1] | B |
| 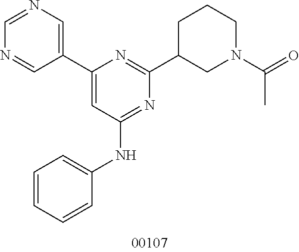 [SMILES: CC(=O)N1CCCC(c2nc(Nc3ccccc3)cc(-c3cncnc3)n2)C1] | B |
| 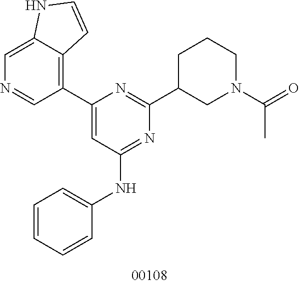 [SMILES: CC(=O)N1CCCC(c2nc(Nc3ccccc3)cc(-c3cncc4[nH]ccc34)n2)C1] | A |
| 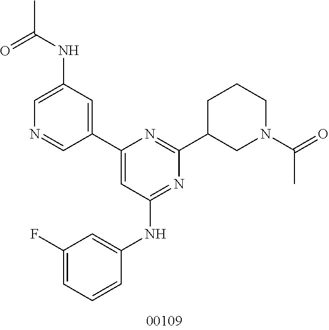 [SMILES: CC(=O)Nc1cncc(-c2cc(Nc3cccc(F)c3)nc(C3CCCN(C(C)=O)C3)n2)c1] | A |
| 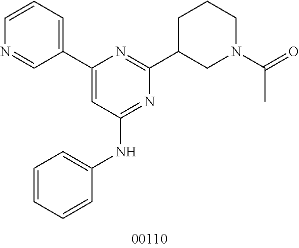 [SMILES: CC(=O)N1CCCC(c2nc(Nc3ccccc3)cc(-c3cccnc3)n2)C1] | B |
| 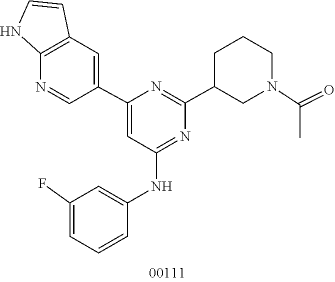 [SMILES: CC(=O)N1CCCC(c2nc(Nc3cccc(F)c3)cc(-c3cnc4[nH]ccc4c3)n2)C1] | B |
| 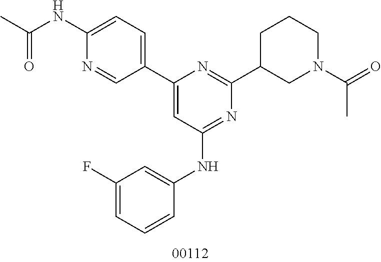 [SMILES: CC(=O)Nc1ccc(-c2cc(Nc3cccc(F)c3)nc(C3CCCN(C(C)=O)C3)n2)cn1] | B |
| 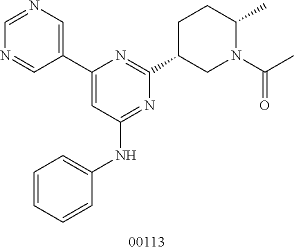 [SMILES: CC(=O)N1C[C@H](c2nc(Nc3ccccc3)cc(-c3cncnc3)n2)CC[C@@H]1C] | A |
| 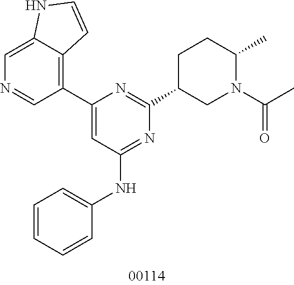 [SMILES: CC(=O)N1C[C@H](c2nc(Nc3ccccc3)cc(-c3cncc4[nH]ccc34)n2)CC[C@@H]1C] | A |
| 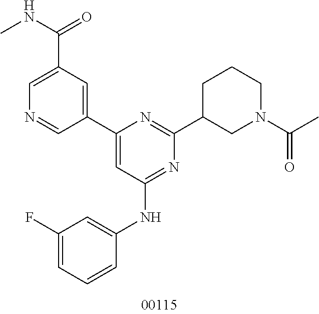 [SMILES: CNC(=O)c1cncc(-c2cc(Nc3cccc(F)c3)nc(C3CCCN(C(C)=O)C3)n2)c1] | A |
| 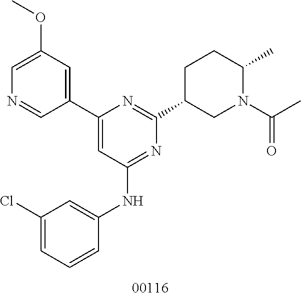 [SMILES: COc1cncc(-c2cc(Nc3cccc(Cl)c3)nc([C@@H]3CC[C@H](C)N(C(C)=O)C3)n2)c1] | A |
| 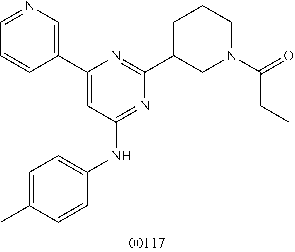 [SMILES: CCC(=O)N1CCCC(c2nc(Nc3ccc(C)cc3)cc(-c3cccnc3)n2)C1] | B |
| 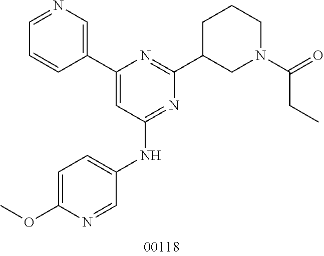 [SMILES: CCC(=O)N1CCCC(c2nc(Nc3ccc(OC)nc3)cc(-c3cccnc3)n2)C1] | C |
| 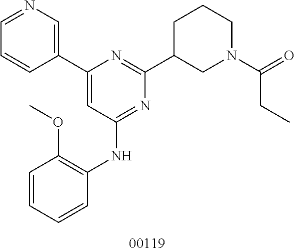 [SMILES: CCC(=O)N1CCCC(c2nc(Nc3ccccc3OC)cc(-c3cccnc3)n2)C1] | C |
| 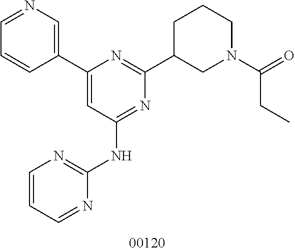 [SMILES: CCC(=O)N1CCCC(c2nc(Nc3ncccn3)cc(-c3cccnc3)n2)C1] | C |
| 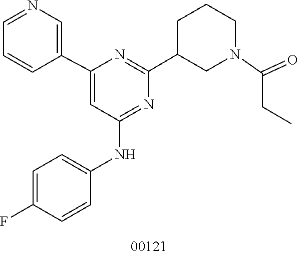 [SMILES: CCC(=O)N1CCCC(c2nc(Nc3ccc(F)cc3)cc(-c3cccnc3)n2)C1] | C |
| 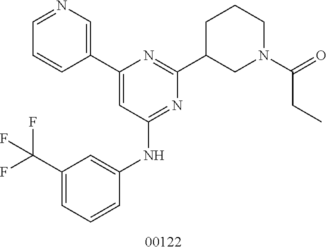 [SMILES: CCC(=O)N1CCCC(c2nc(Nc3cccc(C(F)(F)F)c3)cc(-c3cccnc3)n2)C1] | C |
| 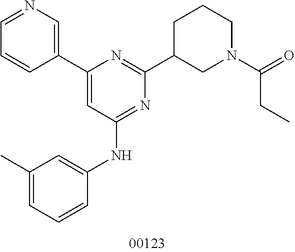 [SMILES: CCC(=O)N1CCCC(c2nc(Nc3cccc(C)c3)cc(-c3cccnc3)n2)C1] | B |
| 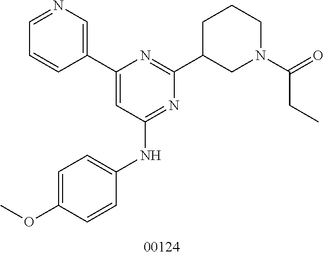 [SMILES: CCC(=O)N1CCCC(c2nc(Nc3ccc(OC)cc3)cc(-c3cccnc3)n2)C1] | B |
| 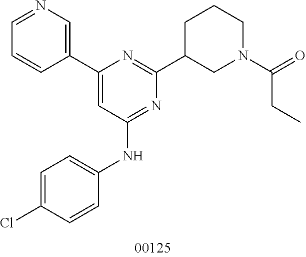 [SMILES: CCC(=O)N1CCCC(c2nc(Nc3ccc(Cl)cc3)cc(-c3cccnc3)n2)C1] | B |
| 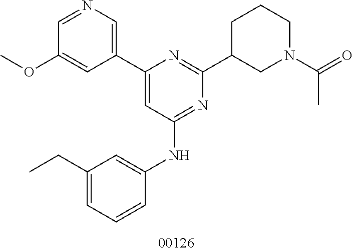 [SMILES: CCc1cccc(Nc2cc(-c3cncc(OC)c3)nc(C3CCCN(C(C)=O)C3)n2)c1] | B |
| 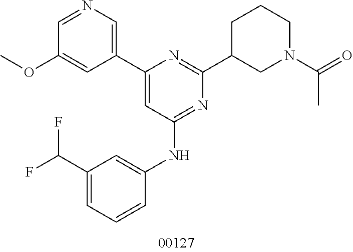 [SMILES: COc1cncc(-c2cc(Nc3cccc(C(F)F)c3)nc(C3CCCN(C(C)=O)C3)n2)c1] | B |
| 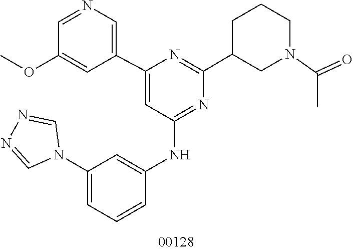 [SMILES: COc1cncc(-c2cc(Nc3cccc(-n4cnnc4)c3)nc(C3CCCN(C(C)=O)C3)n2)c1] | C |
| 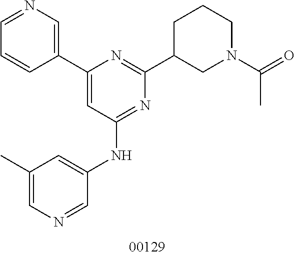 [SMILES: CC(=O)N1CCCC(c2nc(Nc3cncc(C)c3)cc(-c3cccnc3)n2)C1] | B |
| 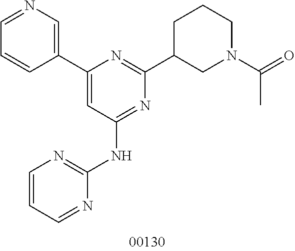 [SMILES: CC(=O)N1CCCC(c2nc(Nc3ncccn3)cc(-c3cccnc3)n2)C1] | C |
| 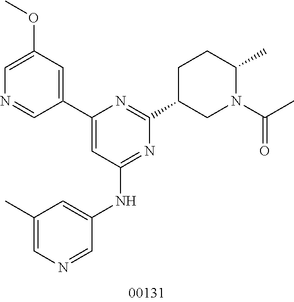 [SMILES: COc1cncc(-c2cc(Nc3cncc(C)c3)nc([C@@H]3CC[C@H](C)N(C(C)=O)C3)n2)c1] | A |
| 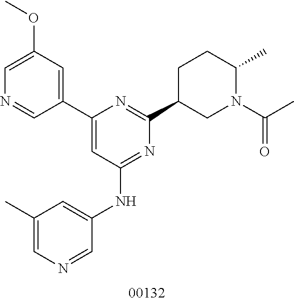 [SMILES: COc1cncc(-c2cc(Nc3cncc(C)c3)nc([C@H]3CC[C@H](C)N(C(C)=O)C3)n2)c1] | C |
| 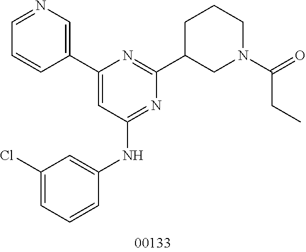 [SMILES: CCC(=O)N1CCCC(c2nc(Nc3cccc(Cl)c3)cc(-c3cccnc3)n2)C1] | B |
| 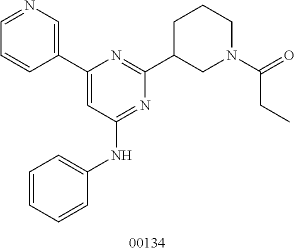 [SMILES: CCC(=O)N1CCCC(c2nc(Nc3ccccc3)cc(-c3cccnc3)n2)C1] | B |
| 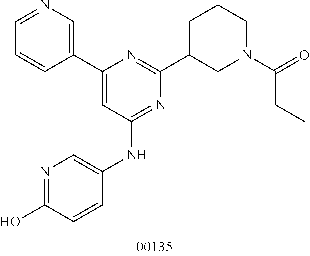 [SMILES: CCC(=O)N1CCCC(c2nc(Nc3ccc(O)nc3)cc(-c3cccnc3)n2)C1] | C |
| 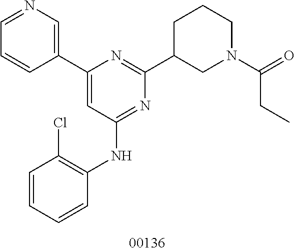 [SMILES: CCC(=O)N1CCCC(c2nc(Nc3ccccc3Cl)cc(-c3cccnc3)n2)C1] | B |
| 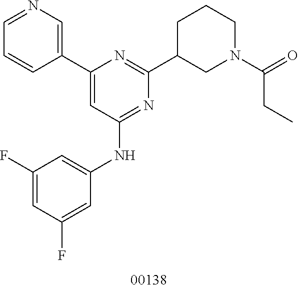 [SMILES: CCC(=O)N1CCCC(c2nc(Nc3cc(F)cc(F)c3)cc(-c3cccnc3)n2)C1] | B |
| 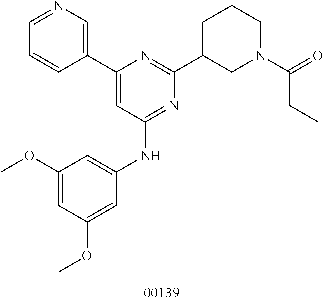 [SMILES: CCC(=O)N1CCCC(c2nc(Nc3cc(OC)cc(OC)c3)cc(-c3cccnc3)n2)C1] | B |
| 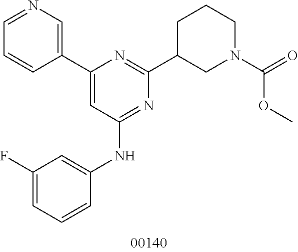 [SMILES: COC(=O)N1CCCC(c2nc(Nc3cccc(F)c3)cc(-c3cccnc3)n2)C1] | B |
| 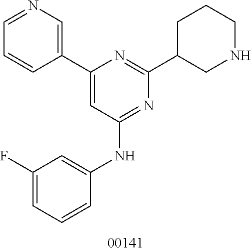 [SMILES: Fc1cccc(Nc2cc(-c3cccnc3)nc(C3CCCNC3)n2)c1] | C |
| 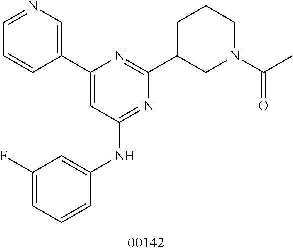 [SMILES: CC(=O)N1CCCC(c2nc(Nc3cccc(F)c3)cc(-c3cccnc3)n2)C1] | A |
| 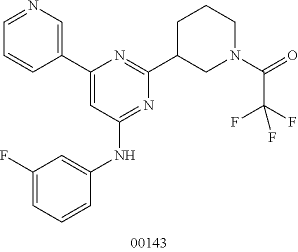 [SMILES: O=C(N1CCCC(c2nc(Nc3cccc(F)c3)cc(-c3cccnc3)n2)C1)C(F)(F)F] | C |
| 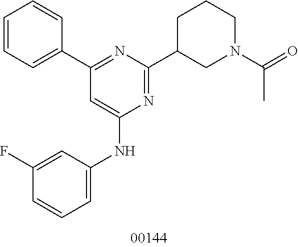 [SMILES: CC(=O)N1CCCC(c2nc(Nc3cccc(F)c3)cc(-c3ccccc3)n2)C1] | B |
| 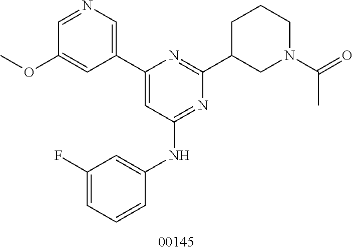 [SMILES: COc1cncc(-c2cc(Nc3cccc(F)c3)nc(C3CCCN(C(C)=O)C3)n2)c1] | A |
| 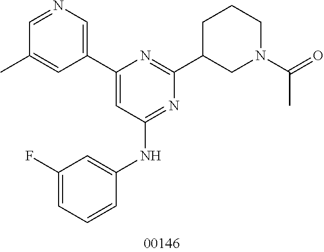 [SMILES: CC(=O)N1CCCC(c2nc(Nc3cccc(F)c3)cc(-c3cncc(C)c3)n2)C1] | A |
| 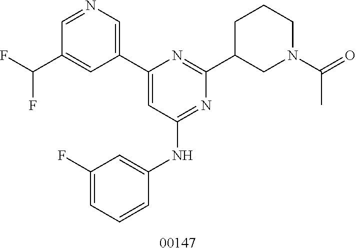 [SMILES: CC(=O)N1CCCC(c2nc(Nc3cccc(F)c3)cc(-c3cncc(C(F)F)c3)n2)C1] | A |
| 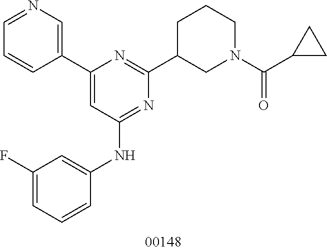 [SMILES: O=C(C1CC1)N1CCCC(c2nc(Nc3cccc(F)c3)cc(-c3cccnc3)n2)C1] | B |
| 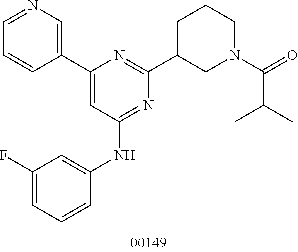 [SMILES: CC(C)C(=O)N1CCCC(c2nc(Nc3cccc(F)c3)cc(-c3cccnc3)n2)C1] | C |
| 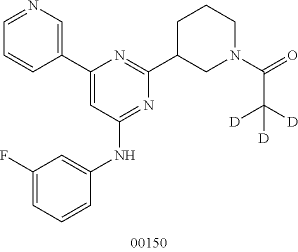 [SMILES: [2H]C([2H])([2H])C(=O)N1CCCC(c2nc(Nc3cccc(F)c3)cc(-c3cccnc3)n2)C1] | A |
| 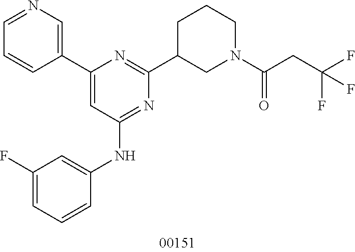 [SMILES: O=C(CC(F)(F)F)N1CCCC(c2nc(Nc3cccc(F)c3)cc(-c3cccnc3)n2)C1] | C |
| 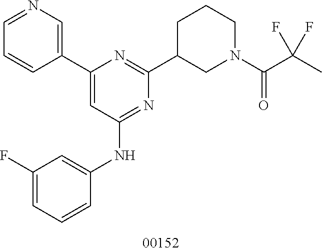 [SMILES: CC(F)(F)C(=O)N1CCCC(c2nc(Nc3cccc(F)c3)cc(-c3cccnc3)n2)C1] | C |
| 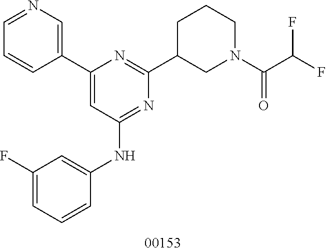 [SMILES: O=C(C(F)F)N1CCCC(c2nc(Nc3cccc(F)c3)cc(-c3cccnc3)n2)C1] | C |
| 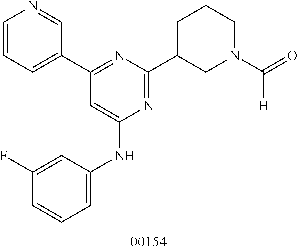 [SMILES: O=CN1CCCC(c2nc(Nc3cccc(F)c3)cc(-c3cccnc3)n2)C1] | C |
| 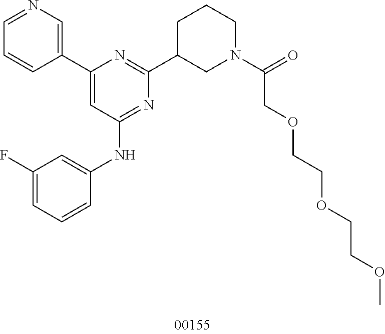 [SMILES: COCCOCCOCC(=O)N1CCCC(c2nc(Nc3cccc(F)c3)cc(-c3cccnc3)n2)C1] | C |
| 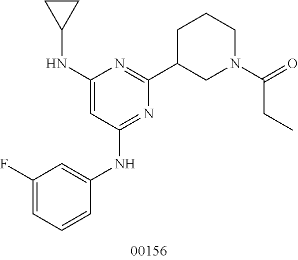 [SMILES: CCC(=O)N1CCCC(c2nc(Nc3cccc(F)c3)cc(NC3CC3)n2)C1] | C |
| 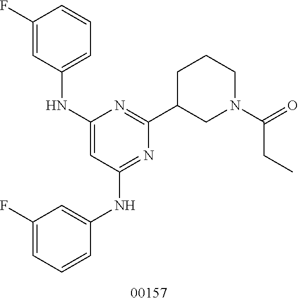 [SMILES: CCC(=O)N1CCCC(c2nc(Nc3cccc(F)c3)cc(Nc3cccc(F)c3)n2)C1] | C |
| 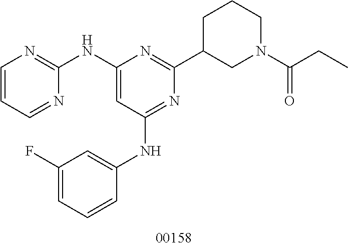 [SMILES: CCC(=O)N1CCCC(c2nc(Nc3cccc(F)c3)cc(Nc3ncccn3)n2)C1] | B |
| 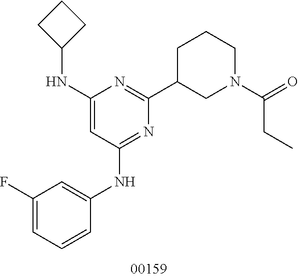 [SMILES: CCC(=O)N1CCCC(c2nc(Nc3cccc(F)c3)cc(NC3CCC3)n2)C1] | C |
| 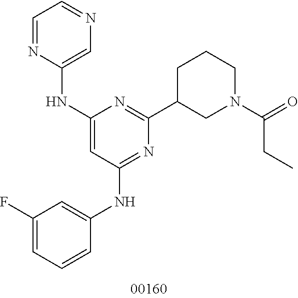 [SMILES: CCC(=O)N1CCCC(c2nc(Nc3cccc(F)c3)cc(Nc3cnccn3)n2)C1] | C |
| 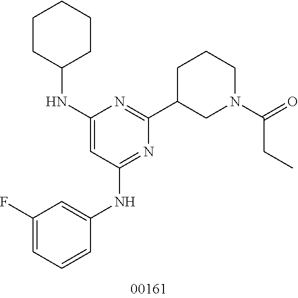 [SMILES: CCC(=O)N1CCCC(c2nc(Nc3cccc(F)c3)cc(NC3CCCCC3)n2)C1] | C |
| 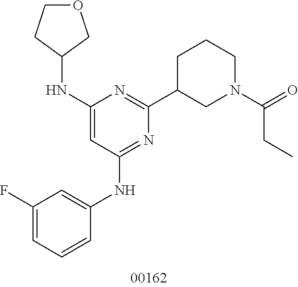 [SMILES: CCC(=O)N1CCCC(c2nc(Nc3cccc(F)c3)cc(NC3CCOC3)n2)C1] | C |
| 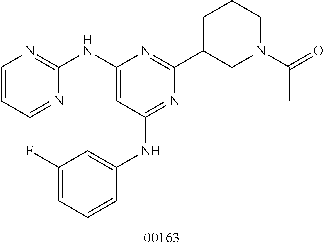 [SMILES: CC(=O)N1CCCC(c2nc(Nc3cccc(F)c3)cc(Nc3ncccn3)n2)C1] | A |
| 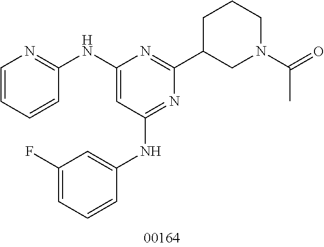 [SMILES: CC(=O)N1CCCC(c2nc(Nc3cccc(F)c3)cc(Nc3ccccn3)n2)C1] | B |
| 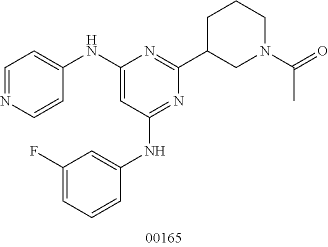 [SMILES: CC(=O)N1CCCC(c2nc(Nc3ccncc3)cc(Nc3cccc(F)c3)n2)C1] | C |
| 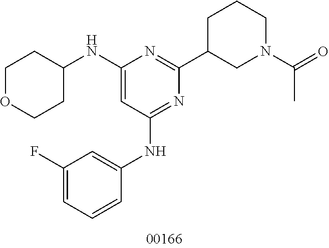 [SMILES: CC(=O)N1CCCC(c2nc(Nc3cccc(F)c3)cc(NC3CCOCC3)n2)C1] | C |
| 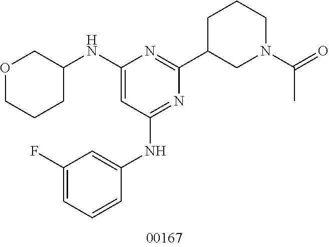 [SMILES: CC(=O)N1CCCC(c2nc(Nc3cccc(F)c3)cc(NC3CCCOC3)n2)C1] | B |
| 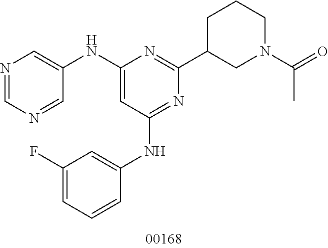 [SMILES: CC(=O)N1CCCC(c2nc(Nc3cncnc3)cc(Nc3cccc(F)c3)n2)C1] | B |
| 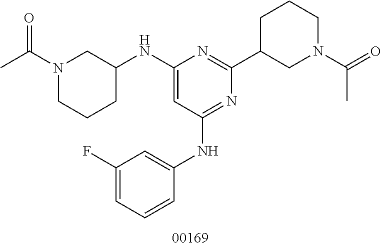 [SMILES: CC(=O)N1CCCC(Nc2cc(Nc3cccc(F)c3)nc(C3CCCN(C(C)=O)C3)n2)C1] | C |
| 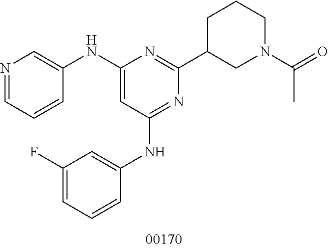 [SMILES: CC(=O)N1CCCC(c2nc(Nc3cccnc3)cc(Nc3cccc(F)c3)n2)C1] | B |
| 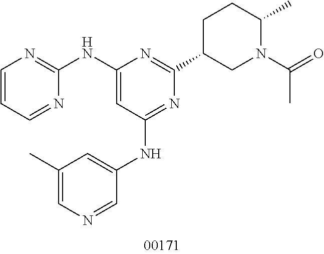 [SMILES: CC(=O)N1C[C@H](c2nc(Nc3cncc(C)c3)cc(Nc3ncccn3)n2)CC[C@@H]1C] | A |
| 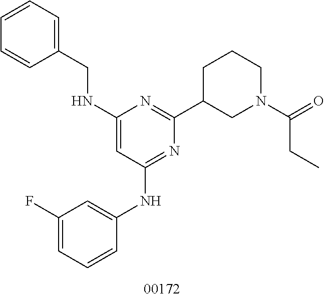 [SMILES: CCC(=O)N1CCCC(c2nc(NCc3ccccc3)cc(Nc3cccc(F)c3)n2)C1] | C |
| 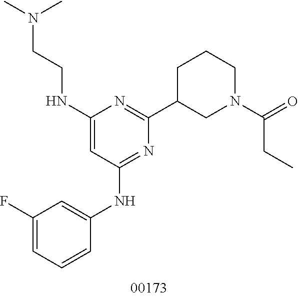 [SMILES: CCC(=O)N1CCCC(c2nc(NCCN(C)C)cc(Nc3cccc(F)c3)n2)C1] | C |
| 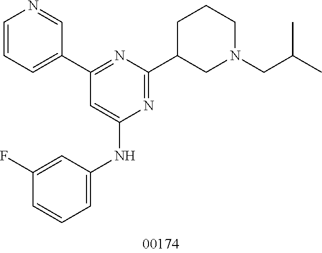 [SMILES: CC(C)CN1CCCC(c2nc(Nc3cccc(F)c3)cc(-c3cccnc3)n2)C1] | C |
| 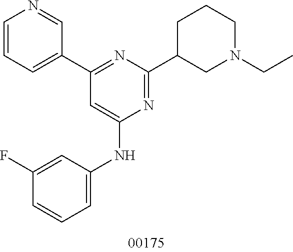 [SMILES: CCN1CCCC(c2nc(Nc3cccc(F)c3)cc(-c3cccnc3)n2)C1] | C |
| 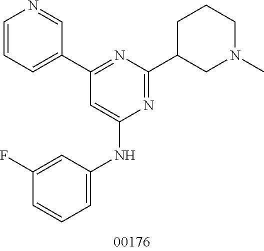 [SMILES: CN1CCCC(c2nc(Nc3cccc(F)c3)cc(-c3cccnc3)n2)C1] | C |
| 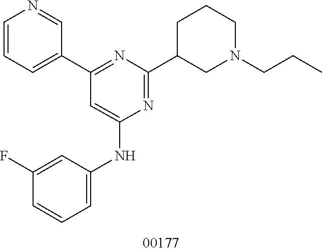 [SMILES: CCCN1CCCC(c2nc(Nc3cccc(F)c3)cc(-c3cccnc3)n2)C1] | C |
| 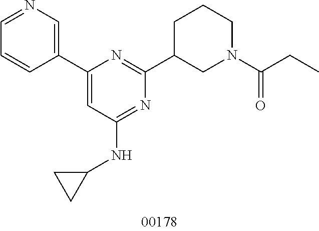 [SMILES: CCC(=O)N1CCCC(c2nc(NC3CC3)cc(-c3cccnc3)n2)C1] | C |
| 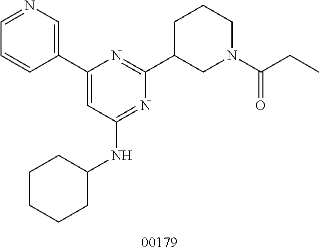 [SMILES: CCC(=O)N1CCCC(c2nc(NC3CCCCC3)cc(-c3cccnc3)n2)C1] | C |
| 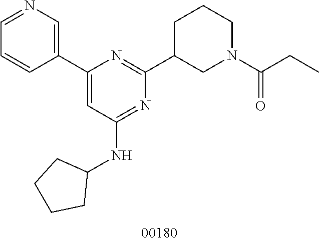 [SMILES: CCC(=O)N1CCCC(c2nc(NC3CCCC3)cc(-c3cccnc3)n2)C1] | C |
| 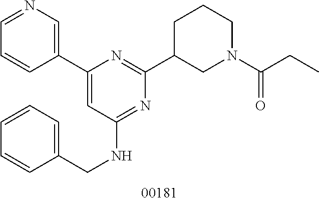 [SMILES: CCC(=O)N1CCCC(c2nc(NCc3ccccc3)cc(-c3cccnc3)n2)C1] | C |
| 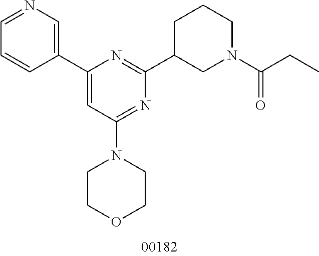 [SMILES: CCC(=O)N1CCCC(c2nc(-c3cccnc3)cc(N3CCOCC3)n2)C1] | C |
| 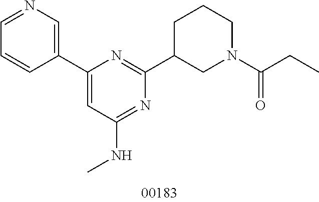 [SMILES: CCC(=O)N1CCCC(c2nc(NC)cc(-c3cccnc3)n2)C1] | C |
| 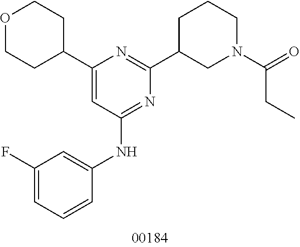 [SMILES: CCC(=O)N1CCCC(c2nc(Nc3cccc(F)c3)cc(C3CCOCC3)n2)C1] | C |
| 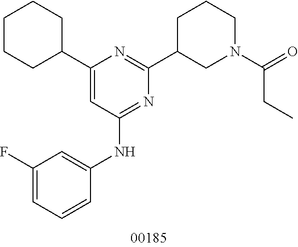 [SMILES: CCC(=O)N1CCCC(c2nc(Nc3cccc(F)c3)cc(C3CCCCC3)n2)C1] | C |
| 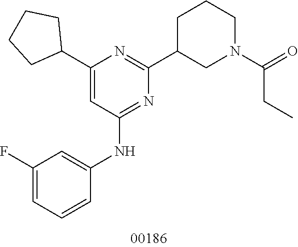 [SMILES: CCC(=O)N1CCCC(c2nc(Nc3cccc(F)c3)cc(C3CCCC3)n2)C1] | C |
| 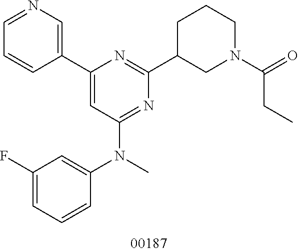 [SMILES: CCC(=O)N1CCCC(c2nc(-c3cccnc3)cc(N(C)c3cccc(F)c3)n2)C1] | B |
| 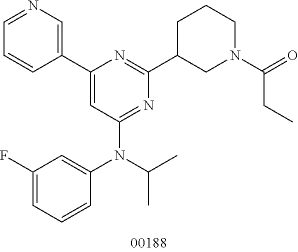 [SMILES: CCC(=O)N1CCCC(c2nc(-c3cccnc3)cc(N(c3cccc(F)c3)C(C)C)n2)C1] | C |
| 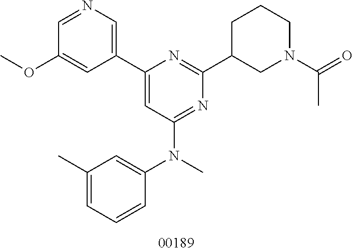 [SMILES: COc1cncc(-c2cc(N(C)c3cccc(C)c3)nc(C3CCCN(C(C)=O)C3)n2)c1] | B |
| 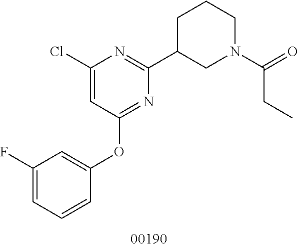 [SMILES: CCC(=O)N1CCCC(c2nc(Cl)cc(Oc3cccc(F)c3)n2)C1] | C |
| 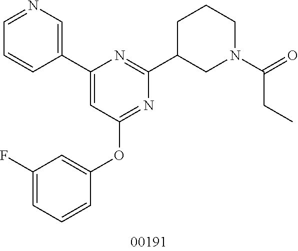 [SMILES: CCC(=O)N1CCCC(c2nc(Oc3cccc(F)c3)cc(-c3cccnc3)n2)C1] | C |
| 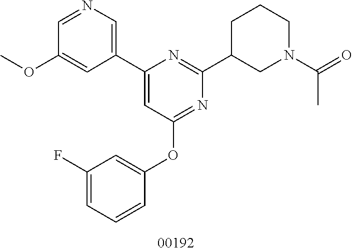 | C |
| 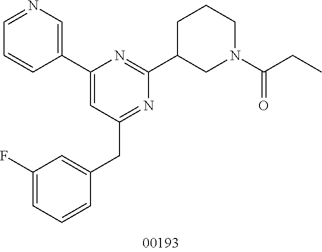 [SMILES: CCC(=O)N1CCCC(c2nc(Cc3cccc(F)c3)cc(-c3cccnc3)n2)C1] | C |
| 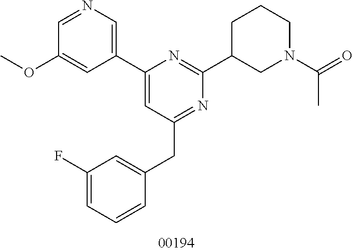 [SMILES: COc1cncc(-c2cc(Cc3cccc(F)c3)nc(C3CCCN(C(C)=O)C3)n2)c1] | C |
| 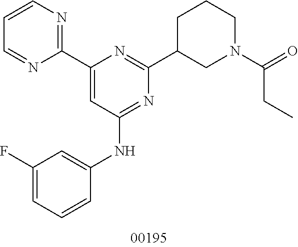 [SMILES: CCC(=O)N1CCCC(c2nc(Nc3cccc(F)c3)cc(-c3ncccn3)n2)C1] | C |
| 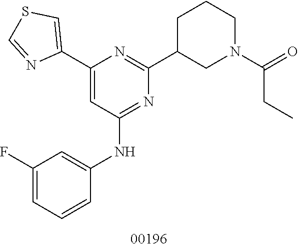 [SMILES: CCC(=O)N1CCCC(c2nc(Nc3cccc(F)c3)cc(-c3cscn3)n2)C1] | C |
| 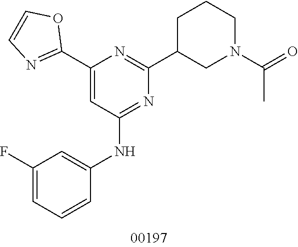 [SMILES: CC(=O)N1CCCC(c2nc(Nc3cccc(F)c3)cc(-c3ncco3)n2)C1] | C |
| 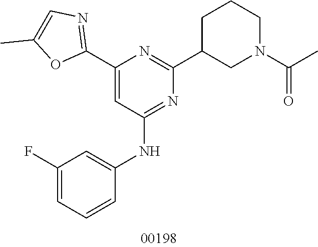 [SMILES: CC(=O)N1CCCC(c2nc(Nc3cccc(F)c3)cc(-c3ncc(C)o3)n2)C1] | C |
| 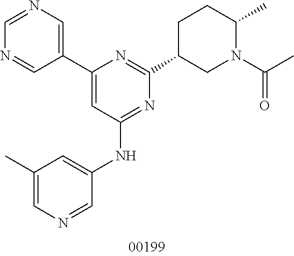 [SMILES: CC(=O)N1C[C@H](c2nc(Nc3cncc(C)c3)cc(-c3cncnc3)n2)CC[C@@H]1C] | A |
| 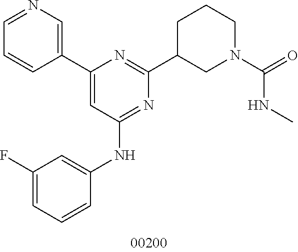 [SMILES: CNC(=O)N1CCCC(c2nc(Nc3cccc(F)c3)cc(-c3cccnc3)n2)C1] | B |
| 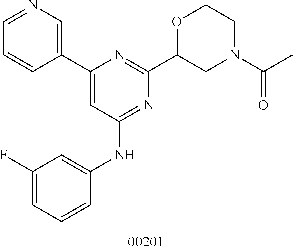 [SMILES: CC(=O)N1CCOC(c2nc(Nc3cccc(F)c3)cc(-c3cccnc3)n2)C1] | C |
| 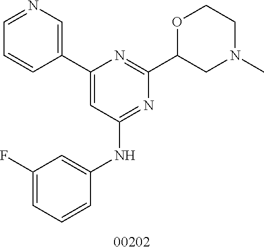 [SMILES: CN1CCOC(c2nc(Nc3cccc(F)c3)cc(-c3cccnc3)n2)C1] | C |
| 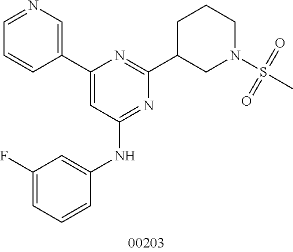 [SMILES: CS(=O)(=O)N1CCCC(c2nc(Nc3cccc(F)c3)cc(-c3cccnc3)n2)C1] | C |
| 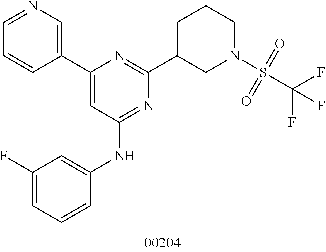 [SMILES: O=S(=O)(N1CCCC(c2nc(Nc3cccc(F)c3)cc(-c3cccnc3)n2)C1)C(F)(F)F] | C |
| 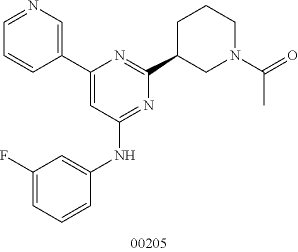 [SMILES: CC(=O)N1CCC[C@H](c2nc(Nc3cccc(F)c3)cc(-c3cccnc3)n2)C1] | C |
| 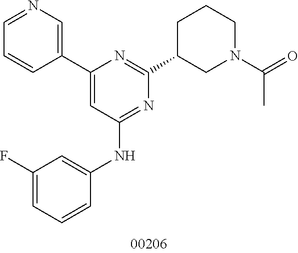 [SMILES: CC(=O)N1CCC[C@@H](c2nc(Nc3cccc(F)c3)cc(-c3cccnc3)n2)C1] | A |
| 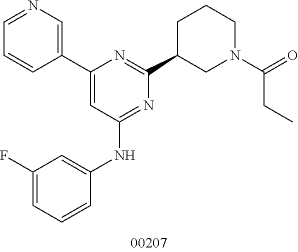 [SMILES: CCC(=O)N1CCC[C@H](c2nc(Nc3cccc(F)c3)cc(-c3cccnc3)n2)C1] | C |
| 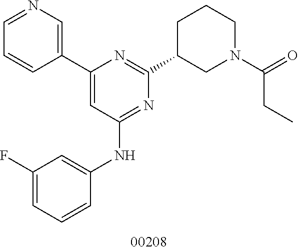 [SMILES: CCC(=O)N1CCC[C@@H](c2nc(Nc3cccc(F)c3)cc(-c3cccnc3)n2)C1] | B |
| 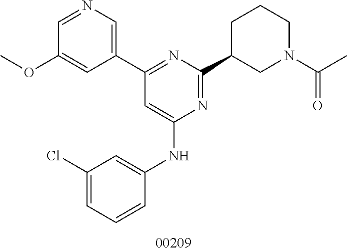 [SMILES: COc1cncc(-c2cc(Nc3cccc(Cl)c3)nc([C@H]3CCCN(C(C)=O)C3)n2)c1] | B |
| 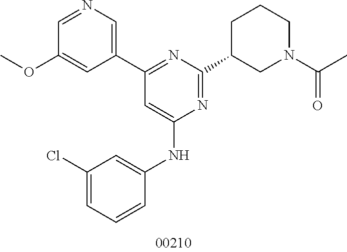 [SMILES: COc1cncc(-c2cc(Nc3cccc(Cl)c3)nc([C@@H]3CCCN(C(C)=O)C3)n2)c1] | A |
| 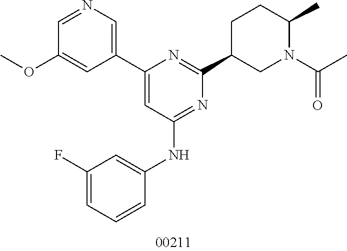 [SMILES: COc1cncc(-c2cc(Nc3cccc(F)c3)nc([C@H]3CC[C@@H](C)N(C(C)=O)C3)n2)c1] | C |
| 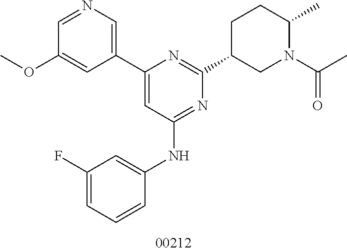 [SMILES: COc1cncc(-c2cc(Nc3cccc(F)c3)nc([C@@H]3CC[C@H](C)N(C(C)=O)C3)n2)c1] | A |
| 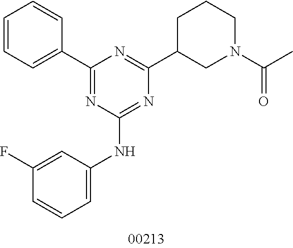 [SMILES: CC(=O)N1CCCC(c2nc(Nc3cccc(F)c3)nc(-c3ccccc3)n2)C1] | B |
| 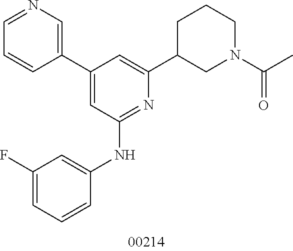 [SMILES: CC(=O)N1CCCC(c2cc(-c3cccnc3)cc(Nc3cccc(F)c3)n2)C1] | B |
| 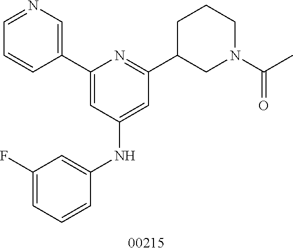 [SMILES: CC(=O)N1CCCC(c2cc(Nc3cccc(F)c3)cc(-c3cccnc3)n2)C1] | B |
| 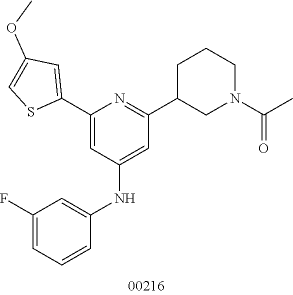 [SMILES: COc1csc(-c2cc(Nc3cccc(F)c3)cc(C3CCCN(C(C)=O)C3)n2)c1] | B |
| 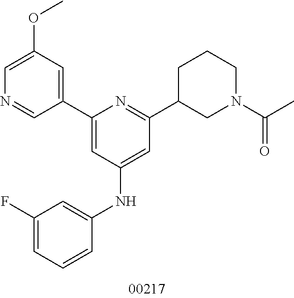 [SMILES: COc1cncc(-c2cc(Nc3cccc(F)c3)cc(C3CCCN(C(C)=O)C3)n2)c1] | B |
| 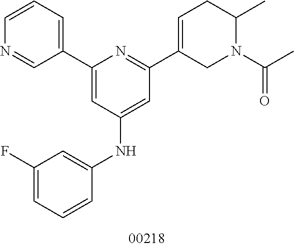 [SMILES: CC(=O)N1CC(c2cc(Nc3cccc(F)c3)cc(-c3cccnc3)n2)=CCC1C] | B |
| 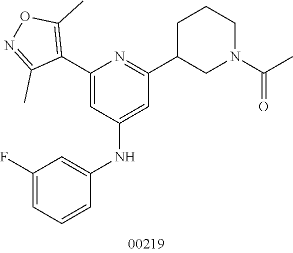 [SMILES: CC(=O)N1CCCC(c2cc(Nc3cccc(F)c3)cc(-c3c(C)noc3C)n2)C1] | B |
| 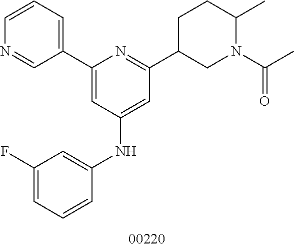 [SMILES: CC(=O)N1CC(c2cc(Nc3cccc(F)c3)cc(-c3cccnc3)n2)CCC1C] | C |
| 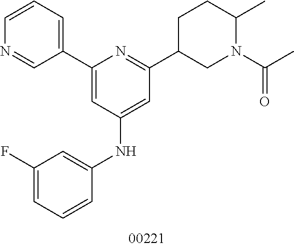 [SMILES: CC(=O)N1CC(c2cc(Nc3cccc(F)c3)cc(-c3cccnc3)n2)CCC1C] | B |
| 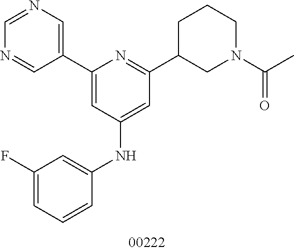 [SMILES: CC(=O)N1CCCC(c2cc(Nc3cccc(F)c3)cc(-c3cncnc3)n2)C1] | B |
| 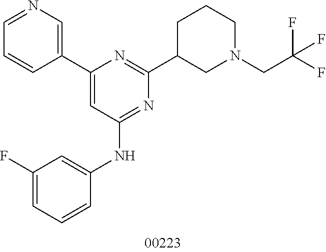 [SMILES: Fc1cccc(Nc2cc(-c3cccnc3)nc(C3CCCN(CC(F)(F)F)C3)n2)c1] | C |
| 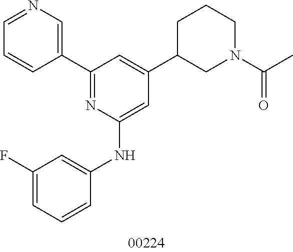 [SMILES: CC(=O)N1CCCC(c2cc(Nc3cccc(F)c3)nc(-c3cccnc3)c2)C1] | C |
| 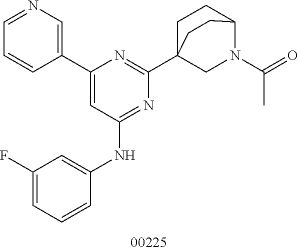 [SMILES: CC(=O)N1CC2(c3nc(Nc4cccc(F)c4)cc(-c4cccnc4)n3)CCC1CC2] | C |
| 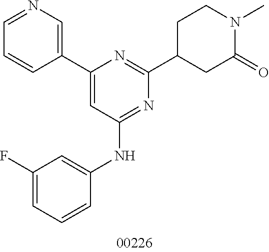 [SMILES: CN1CCC(c2nc(Nc3cccc(F)c3)cc(-c3cccnc3)n2)CC1=O] | C |
| 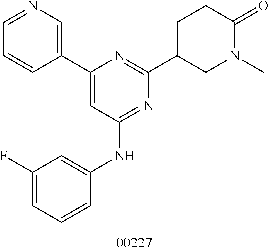 [SMILES: CN1CC(c2nc(Nc3cccc(F)c3)cc(-c3cccnc3)n2)CCC1=O] | C |
| 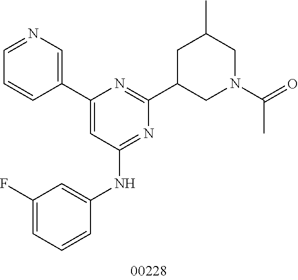 [SMILES: CC(=O)N1CC(C)CC(c2nc(Nc3cccc(F)c3)cc(-c3cccnc3)n2)C1] | A |
| 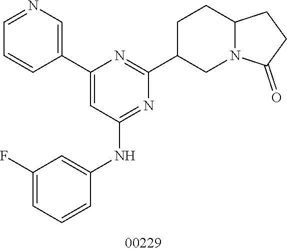 [SMILES: O=C1CCC2CCC(c3nc(Nc4cccc(F)c4)cc(-c4cccnc4)n3)CN12] | C |
| 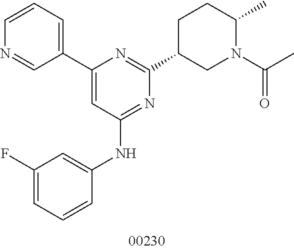 [SMILES: CC(=O)N1C[C@H](c2nc(Nc3cccc(F)c3)cc(-c3cccnc3)n2)CC[C@@H]1C] | A |
| 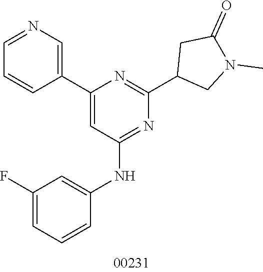 [SMILES: CN1CC(c2nc(Nc3cccc(F)c3)cc(-c3cccnc3)n2)CC1=O] | C |
| 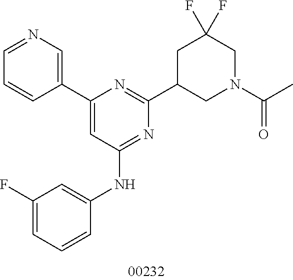 [SMILES: CC(=O)N1CC(c2nc(Nc3cccc(F)c3)cc(-c3cccnc3)n2)CC(F)(F)C1] | C |
| 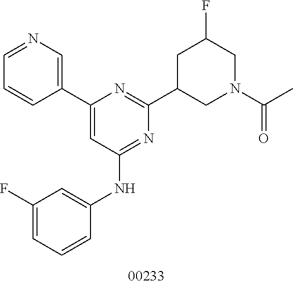 [SMILES: CC(=O)N1CC(F)CC(c2nc(Nc3cccc(F)c3)cc(-c3cccnc3)n2)C1] | B |
| 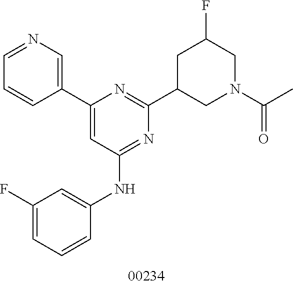 [SMILES: CC(=O)N1CC(F)CC(c2nc(Nc3cccc(F)c3)cc(-c3cccnc3)n2)C1] | B |
| 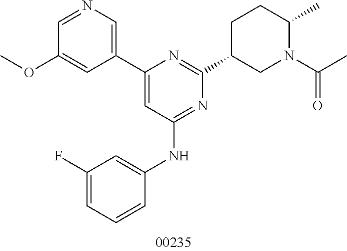 [SMILES: COc1cncc(-c2cc(Nc3cccc(F)c3)nc([C@@H]3CC[C@H](C)N(C(C)=O)C3)n2)c1] | A |
| 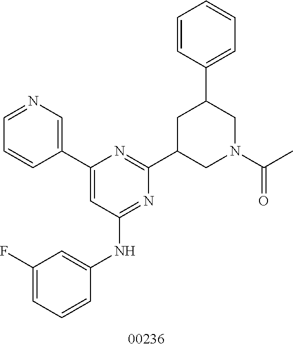 [SMILES: CC(=O)N1CC(c2ccccc2)CC(c2nc(Nc3cccc(F)c3)cc(-c3cccnc3)n2)C1] | A |
| 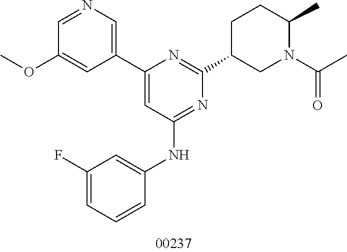 [SMILES: COc1cncc(-c2cc(Nc3cccc(F)c3)nc([C@@H]3CC[C@@H](C)N(C(C)=O)C3)n2)c1] | C |
| 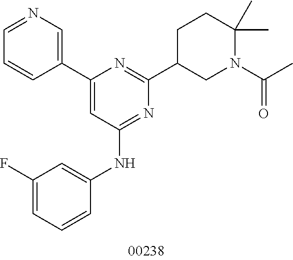 [SMILES: CC(=O)N1CC(c2nc(Nc3cccc(F)c3)cc(-c3cccnc3)n2)CCC1(C)C] | C |
| 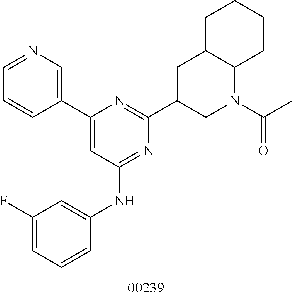 [SMILES: CC(=O)N1CC(c2nc(Nc3cccc(F)c3)cc(-c3cccnc3)n2)CC2CCCCC21] | B |
| 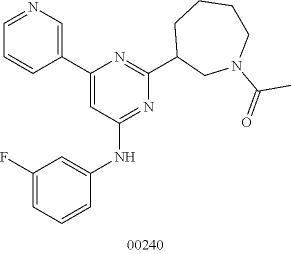 [SMILES: CC(=O)N1CCCCC(c2nc(Nc3cccc(F)c3)cc(-c3cccnc3)n2)C1] | A |
| 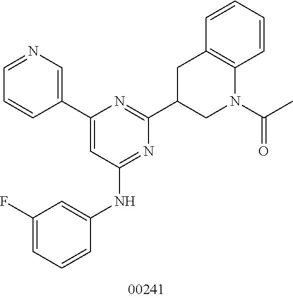 [SMILES: CC(=O)N1CC(c2nc(Nc3cccc(F)c3)cc(-c3cccnc3)n2)Cc2ccccc21] | C |
| 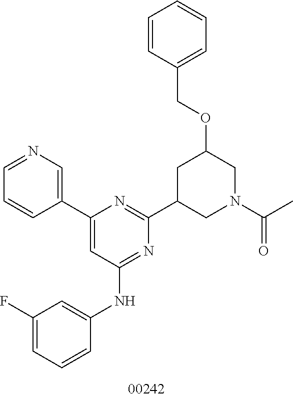 [SMILES: CC(=O)N1CC(OCc2ccccc2)CC(c2nc(Nc3cccc(F)c3)cc(-c3cccnc3)n2)C1] | B |
| 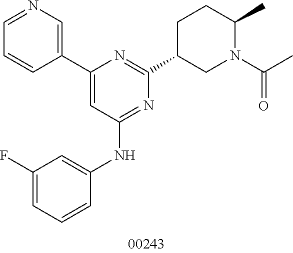 [SMILES: CC(=O)N1C[C@H](c2nc(Nc3cccc(F)c3)cc(-c3cccnc3)n2)CC[C@H]1C] | C |
| 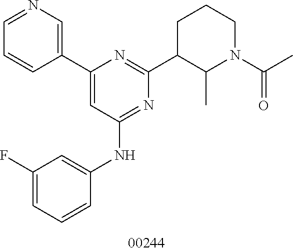 [SMILES: CC(=O)N1CCCC(c2nc(Nc3cccc(F)c3)cc(-c3cccnc3)n2)C1C] | C |
| 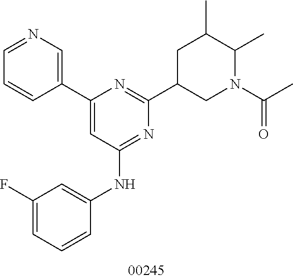 [SMILES: CC(=O)N1CC(c2nc(Nc3cccc(F)c3)cc(-c3cccnc3)n2)CC(C)C1C] | A |
| 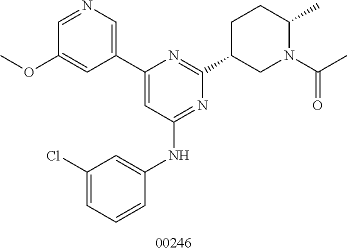 [SMILES: COc1cncc(-c2cc(Nc3cccc(Cl)c3)nc([C@@H]3CC[C@H](C)N(C(C)=O)C3)n2)c1] | A |
| 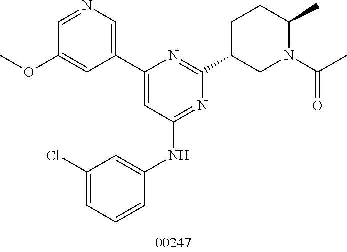 [SMILES: COc1cncc(-c2cc(Nc3cccc(Cl)c3)nc([C@@H]3CC[C@@H](C)N(C(C)=O)C3)n2)c1] | B |
| 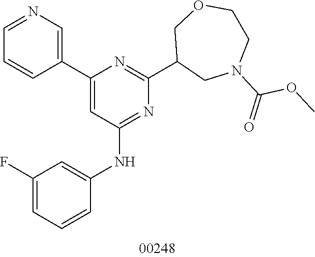 [SMILES: COC(=O)N1CCOCC(c2nc(Nc3cccc(F)c3)cc(-c3cccnc3)n2)C1] | B |
| 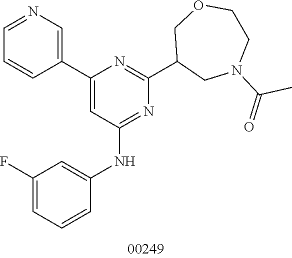 [SMILES: CC(=O)N1CCOCC(c2nc(Nc3cccc(F)c3)cc(-c3cccnc3)n2)C1] | B |
| 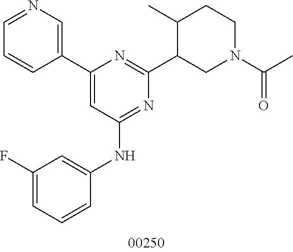 [SMILES: CC(=O)N1CCC(C)C(c2nc(Nc3cccc(F)c3)cc(-c3cccnc3)n2)C1] | B |
| 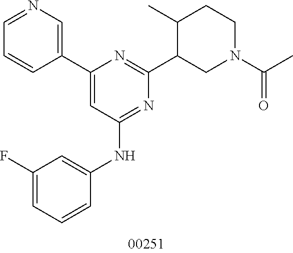 [SMILES: CC(=O)N1CCC(C)C(c2nc(Nc3cccc(F)c3)cc(-c3cccnc3)n2)C1] | B |
| 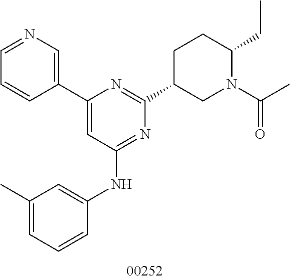 [SMILES: CC[C@H]1CC[C@@H](c2nc(Nc3cccc(C)c3)cc(-c3cccnc3)n2)CN1C(C)=O] | B |
| 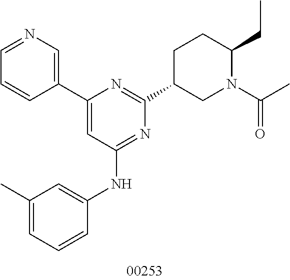 [SMILES: CC[C@@H]1CC[C@@H](c2nc(Nc3cccc(C)c3)cc(-c3cccnc3)n2)CN1C(C)=O] | C |
| 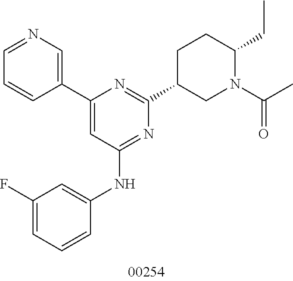 [SMILES: CC[C@H]1CC[C@@H](c2nc(Nc3cccc(F)c3)cc(-c3cccnc3)n2)CN1C(C)=O] | B |
| 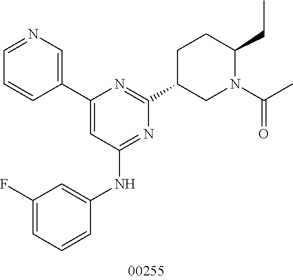 [SMILES: CC[C@@H]1CC[C@@H](c2nc(Nc3cccc(F)c3)cc(-c3cccnc3)n2)CN1C(C)=O] | C |
| 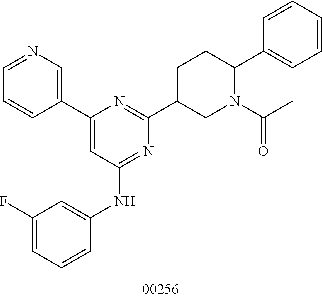 [SMILES: CC(=O)N1CC(c2nc(Nc3cccc(F)c3)cc(-c3cccnc3)n2)CCC1c1ccccc1] | C |
| 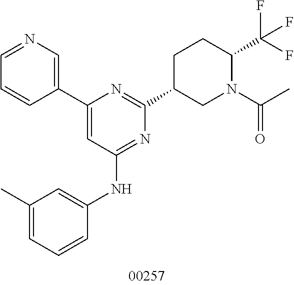 [SMILES: CC(=O)N1C[C@H](c2nc(Nc3cccc(C)c3)cc(-c3cccnc3)n2)CC[C@@H]1C(F)(F)F] | A |
| 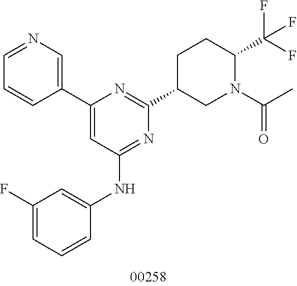 [SMILES: CC(=O)N1C[C@H](c2nc(Nc3cccc(F)c3)cc(-c3cccnc3)n2)CC[C@@H]1C(F)(F)F] | A |
| 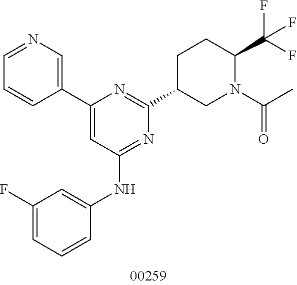 [SMILES: CC(=O)N1C[C@H](c2nc(Nc3cccc(F)c3)cc(-c3cccnc3)n2)CC[C@H]1C(F)(F)F] | C |
| 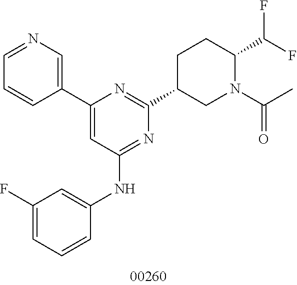 [SMILES: CC(=O)N1C[C@H](c2nc(Nc3cccc(F)c3)cc(-c3cccnc3)n2)CC[C@@H]1C(F)F] | B |
| 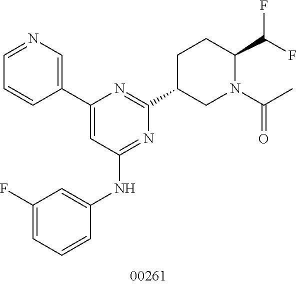 [SMILES: CC(=O)N1C[C@H](c2nc(Nc3cccc(F)c3)cc(-c3cccnc3)n2)CC[C@H]1C(F)F] | C |
| 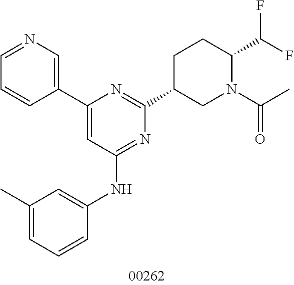 [SMILES: CC(=O)N1C[C@H](c2nc(Nc3cccc(C)c3)cc(-c3cccnc3)n2)CC[C@@H]1C(F)F] | B |
| 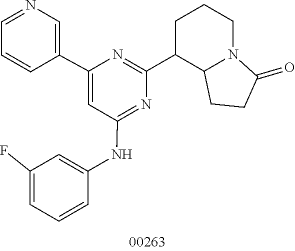 [SMILES: O=C1CCC2C(c3nc(Nc4cccc(F)c4)cc(-c4cccnc4)n3)CCCN12] | B |
| 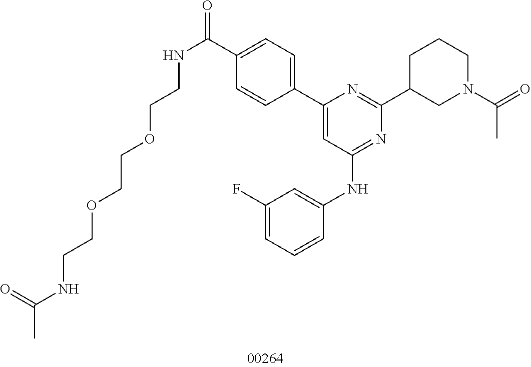 [SMILES: CC(=O)NCCOCCOCCNC(=O)c1ccc(-c2cc(Nc3cccc(F)c3)nc(C3CCCN(C(C)=O)C3)n2)cc1] | C |
| 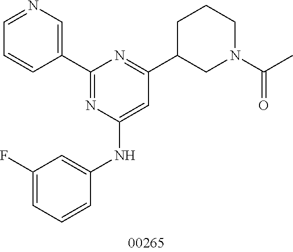 [SMILES: CC(=O)N1CCCC(c2cc(Nc3cccc(F)c3)nc(-c3cccnc3)n2)C1] | B |
| 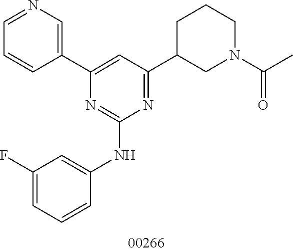 [SMILES: CC(=O)N1CCCC(c2cc(-c3cccnc3)nc(Nc3cccc(F)c3)n2)C1] | B |
| 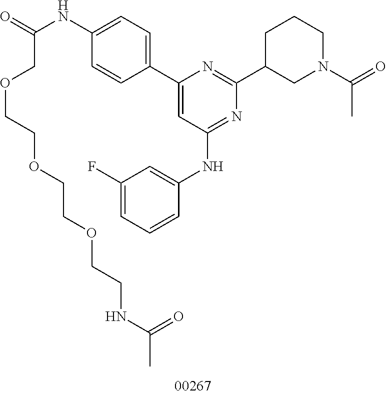 [SMILES: CC(=O)NCCOCCOCCOCC(=O)Nc1ccc(-c2cc(Nc3cccc(F)c3)nc(C3CCCN(C(C)=O)C3)n2)cc1] | B |
| 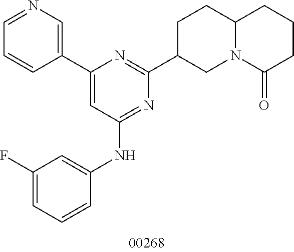 [SMILES: O=C1CCCC2CCC(c3nc(Nc4cccc(F)c4)cc(-c4cccnc4)n3)CN12] | C |
| 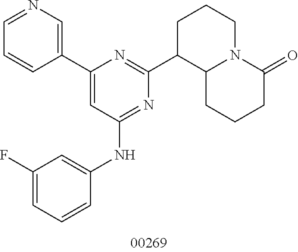 [SMILES: O=C1CCCC2C(c3nc(Nc4cccc(F)c4)cc(-c4cccnc4)n3)CCCN12] | B |
| 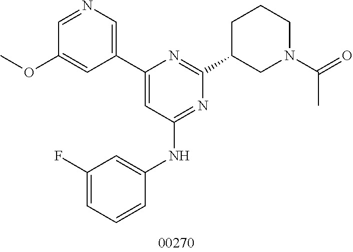 [SMILES: COc1cncc(-c2cc(Nc3cccc(F)c3)nc([C@@H]3CCCN(C(C)=O)C3)n2)c1] | A |
| 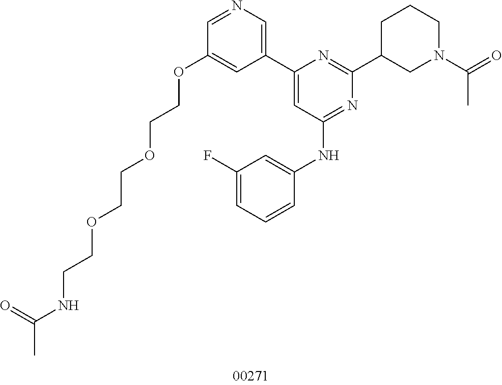 [SMILES: CC(=O)NCCOCCOCCOc1cncc(-c2cc(Nc3cccc(F)c3)nc(C3CCCN(C(C)=O)C3)n2)c1] | B |
| 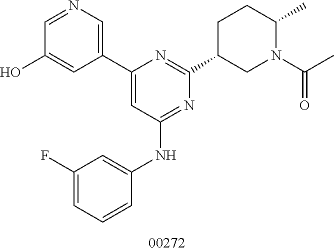 [SMILES: CC(=O)N1C[C@H](c2nc(Nc3cccc(F)c3)cc(-c3cncc(O)c3)n2)CC[C@@H]1C] | A |
| 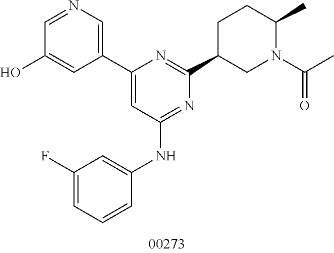 [SMILES: CC(=O)N1C[C@@H](c2nc(Nc3cccc(F)c3)cc(-c3cncc(O)c3)n2)CC[C@H]1C] | C |
| 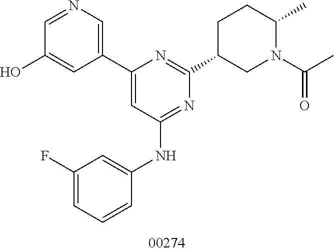 [SMILES: CC(=O)N1C[C@H](c2nc(Nc3cccc(F)c3)cc(-c3cncc(O)c3)n2)CC[C@@H]1C] | A |
| 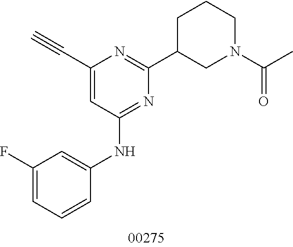 [SMILES: C#Cc1cc(Nc2cccc(F)c2)nc(C2CCCN(C(C)=O)C2)n1] | C |
| 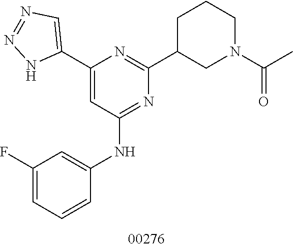 [SMILES: CC(=O)N1CCCC(c2nc(Nc3cccc(F)c3)cc(-c3cnn[nH]3)n2)C1] | C |
| 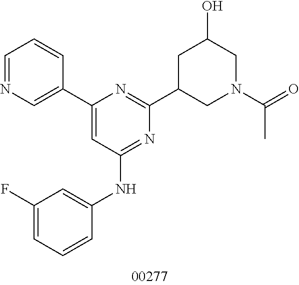 [SMILES: CC(=O)N1CC(O)CC(c2nc(Nc3cccc(F)c3)cc(-c3cccnc3)n2)C1] | B |
| 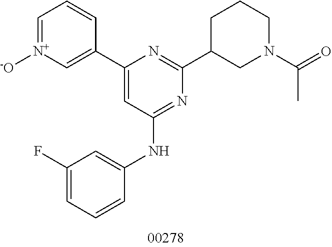 | C |
| 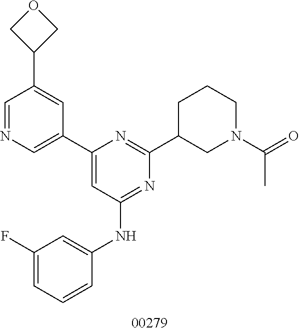 [SMILES: CC(=O)N1CCCC(c2nc(Nc3cccc(F)c3)cc(-c3cncc(C4COC4)c3)n2)C1] | A |
| 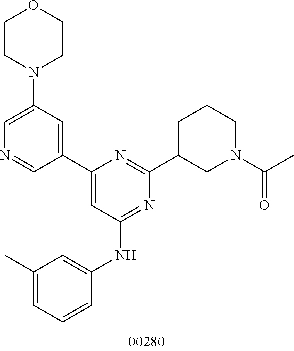 [SMILES: CC(=O)N1CCCC(c2nc(Nc3cccc(C)c3)cc(-c3cncc(N4CCOCC4)c3)n2)C1] | A |
| 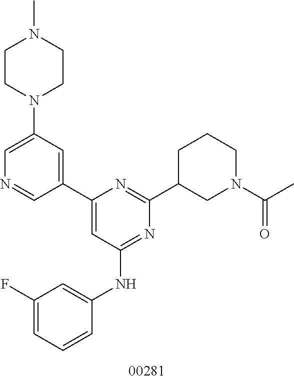 [SMILES: CC(=O)N1CCCC(c2nc(Nc3cccc(F)c3)cc(-c3cncc(N4CCN(C)CC4)c3)n2)C1] | C |
| 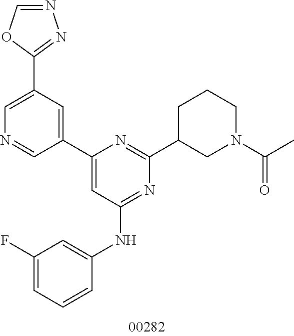 | A |
| 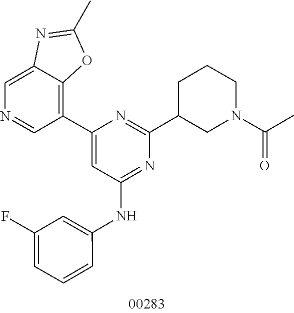 [SMILES: CC(=O)N1CCCC(c2nc(Nc3cccc(F)c3)cc(-c3cncc4nc(C)oc34)n2)C1] | A |
| 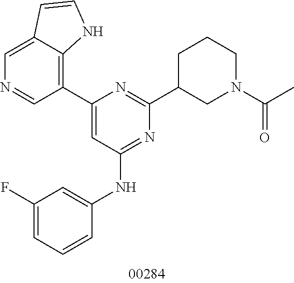 [SMILES: CC(=O)N1CCCC(c2nc(Nc3cccc(F)c3)cc(-c3cncc4cc[nH]c34)n2)C1] | C |
| 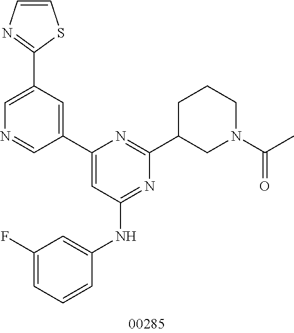 [SMILES: CC(=O)N1CCCC(c2nc(Nc3cccc(F)c3)cc(-c3cncc(-c4nccs4)c3)n2)C1] | A |
| 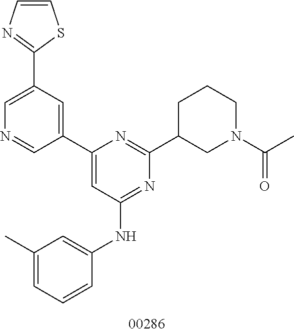 [SMILES: CC(=O)N1CCCC(c2nc(Nc3cccc(C)c3)cc(-c3cncc(-c4nccs4)c3)n2)C1] | A |
| 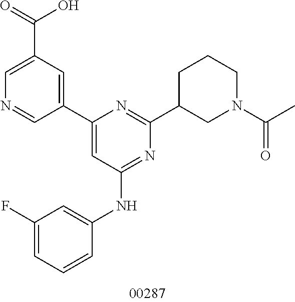 [SMILES: CC(=O)N1CCCC(c2nc(Nc3cccc(F)c3)cc(-c3cncc(C(=O)O)c3)n2)C1] | C |
| 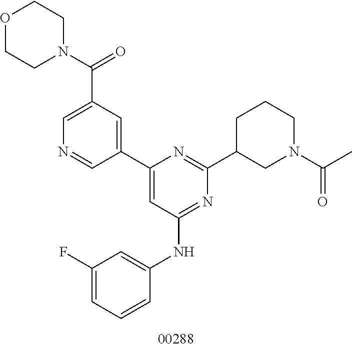 [SMILES: CC(=O)N1CCCC(c2nc(Nc3cccc(F)c3)cc(-c3cncc(C(=O)N4CCOCC4)c3)n2)C1] | B |
| 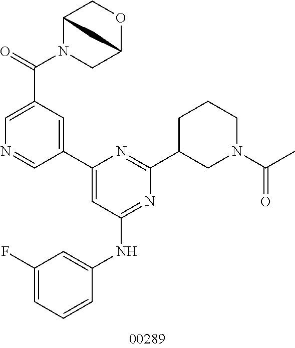 [SMILES: CC(=O)N1CCCC(c2nc(Nc3cccc(F)c3)cc(-c3cncc(C(=O)N4C[C@@H]5C[C@H]4CO5)c3)n2)C1] | B |
| 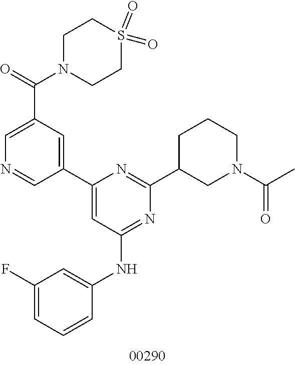 [SMILES: CC(=O)N1CCCC(c2nc(Nc3cccc(F)c3)cc(-c3cncc(C(=O)N4CCS(=O)(=O)CC4)c3)n2)C1] | C |
| 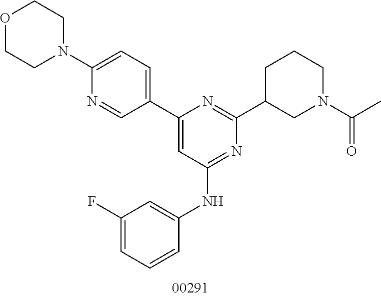 [SMILES: CC(=O)N1CCCC(c2nc(Nc3cccc(F)c3)cc(-c3ccc(N4CCOCC4)nc3)n2)C1] | B |
| 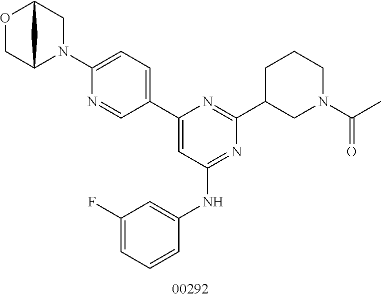 [SMILES: CC(=O)N1CCCC(c2nc(Nc3cccc(F)c3)cc(-c3ccc(N4C[C@@H]5C[C@H]4CO5)nc3)n2)C1] | B |
| 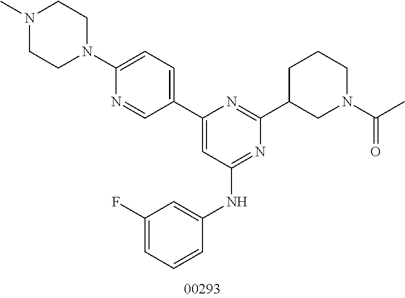 [SMILES: CC(=O)N1CCCC(c2nc(Nc3cccc(F)c3)cc(-c3ccc(N4CCN(C)CC4)nc3)n2)C1] | C |
| 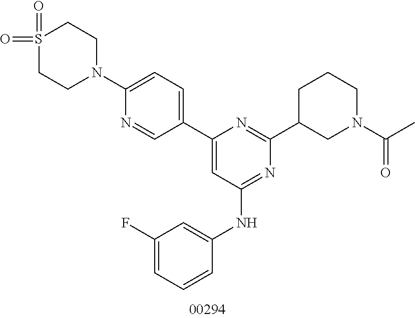 [SMILES: CC(=O)N1CCCC(c2nc(Nc3cccc(F)c3)cc(-c3ccc(N4CCS(=O)(=O)CC4)nc3)n2)C1] | C |
| 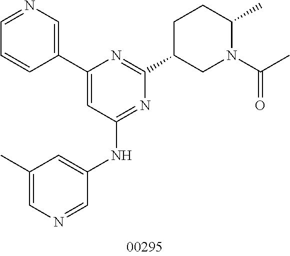 [SMILES: CC(=O)N1C[C@H](c2nc(Nc3cncc(C)c3)cc(-c3cccnc3)n2)CC[C@@H]1C] | A |
| 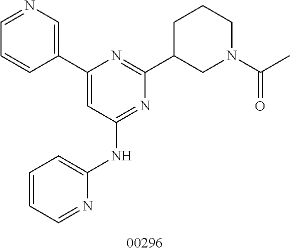 [SMILES: CC(=O)N1CCCC(c2nc(Nc3ccccn3)cc(-c3cccnc3)n2)C1] | C |
| 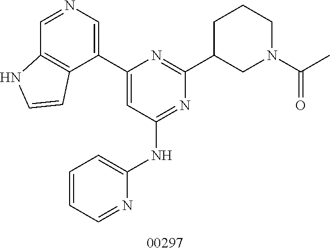 [SMILES: CC(=O)N1CCCC(c2nc(Nc3ccccn3)cc(-c3cncc4[nH]ccc34)n2)C1] | B |
| 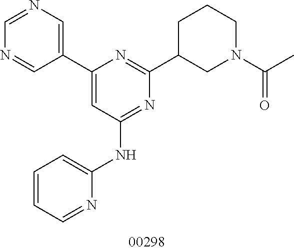 [SMILES: CC(=O)N1CCCC(c2nc(Nc3ccccn3)cc(-c3cncnc3)n2)C1] | C |
| 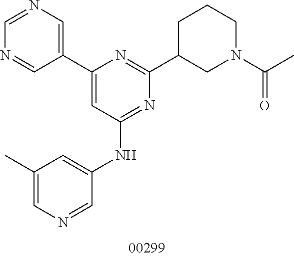 [SMILES: CC(=O)N1CCCC(c2nc(Nc3cncc(C)c3)cc(-c3cncnc3)n2)C1] | B |
| 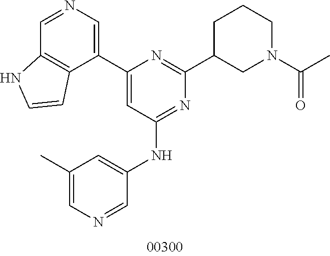 [SMILES: CC(=O)N1CCCC(c2nc(Nc3cncc(C)c3)cc(-c3cncc4[nH]ccc34)n2)C1] | B |
| 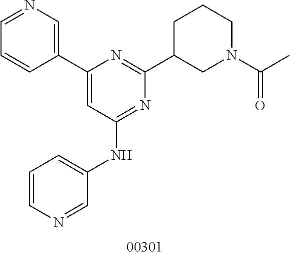 [SMILES: CC(=O)N1CCCC(c2nc(Nc3cccnc3)cc(-c3cccnc3)n2)C1] | B |
| 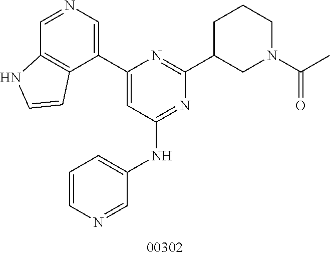 [SMILES: CC(=O)N1CCCC(c2nc(Nc3cccnc3)cc(-c3cncc4[nH]ccc34)n2)C1] | C |
| 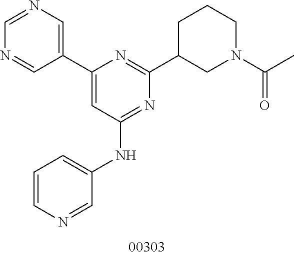 [SMILES: CC(=O)N1CCCC(c2nc(Nc3cccnc3)cc(-c3cncnc3)n2)C1] | C |
| 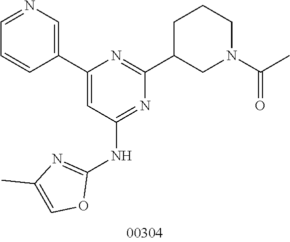 [SMILES: CC(=O)N1CCCC(c2nc(Nc3nc(C)co3)cc(-c3cccnc3)n2)C1] | C |
| 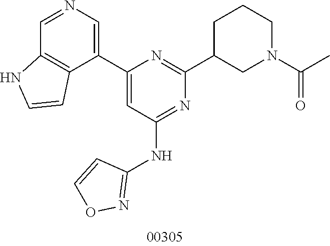 [SMILES: CC(=O)N1CCCC(c2nc(Nc3ccon3)cc(-c3cncc4[nH]ccc34)n2)C1] | C |
| 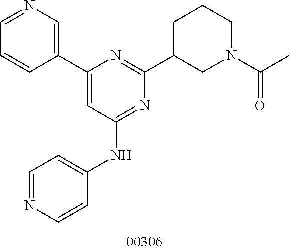 [SMILES: CC(=O)N1CCCC(c2nc(Nc3ccncc3)cc(-c3cccnc3)n2)C1] | B |
| 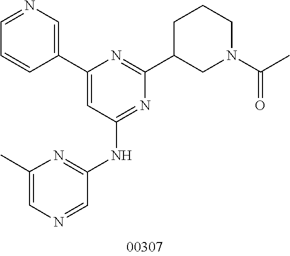 [SMILES: CC(=O)N1CCCC(c2nc(Nc3cncc(C)n3)cc(-c3cccnc3)n2)C1] | B |
| 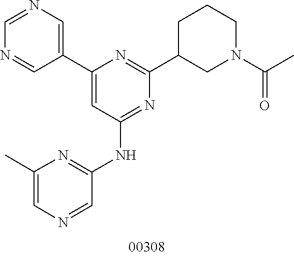 [SMILES: CC(=O)N1CCCC(c2nc(Nc3cncc(C)n3)cc(-c3cncnc3)n2)C1] | C |
| 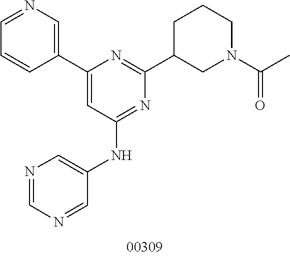 [SMILES: CC(=O)N1CCCC(c2nc(Nc3cncnc3)cc(-c3cccnc3)n2)C1] | C |
| 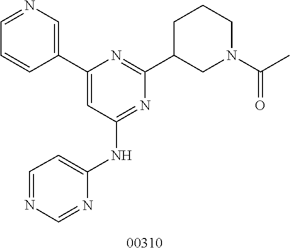 [SMILES: CC(=O)N1CCCC(c2nc(Nc3ccncn3)cc(-c3cccnc3)n2)C1] | C |
| 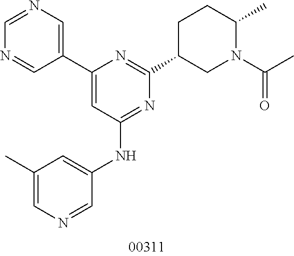 [SMILES: CC(=O)N1C[C@H](c2nc(Nc3cncc(C)c3)cc(-c3cncnc3)n2)CC[C@@H]1C] | B |
| 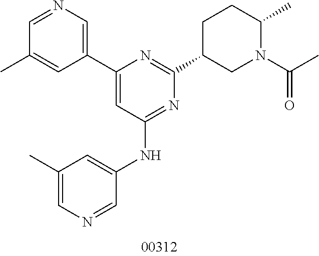 [SMILES: CC(=O)N1C[C@H](c2nc(Nc3cncc(C)c3)cc(-c3cncc(C)c3)n2)CC[C@@H]1C] | A |
| 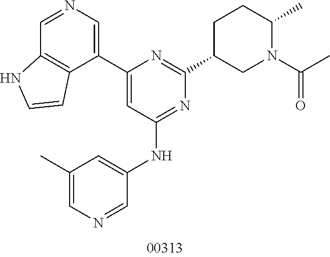 [SMILES: CC(=O)N1C[C@H](c2nc(Nc3cncc(C)c3)cc(-c3cncc4[nH]ccc34)n2)CC[C@@H]1C] | A |
| 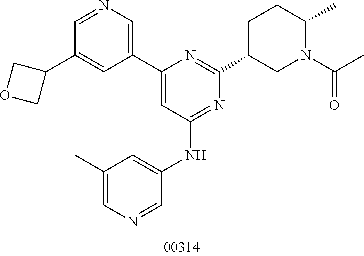 [SMILES: CC(=O)N1C[C@H](c2nc(Nc3cncc(C)c3)cc(-c3cncc(C4COC4)c3)n2)CC[C@@H]1C] | A |
| 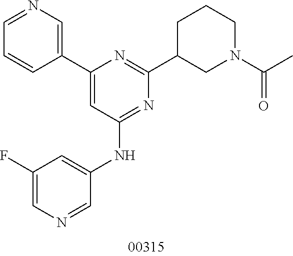 [SMILES: CC(=O)N1CCCC(c2nc(Nc3cncc(F)c3)cc(-c3cccnc3)n2)C1] | B |
| 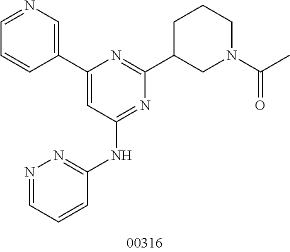 [SMILES: CC(=O)N1CCCC(c2nc(Nc3cccnn3)cc(-c3cccnc3)n2)C1] | C |
| 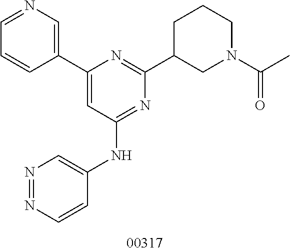 [SMILES: CC(=O)N1CCCC(c2nc(Nc3ccnnc3)cc(-c3cccnc3)n2)C1] | C |
| 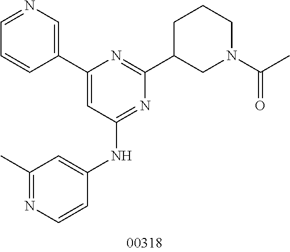 [SMILES: CC(=O)N1CCCC(c2nc(Nc3ccnc(C)c3)cc(-c3cccnc3)n2)C1] | B |
| 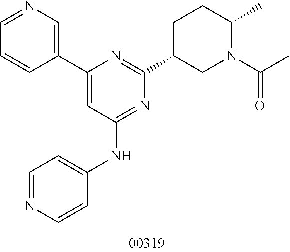 [SMILES: CC(=O)N1C[C@H](c2nc(Nc3ccncc3)cc(-c3cccnc3)n2)CC[C@@H]1C] | A |
| 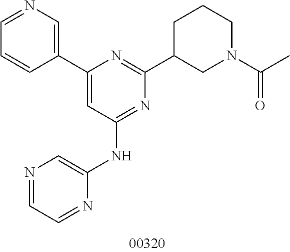 [SMILES: CC(=O)N1CCCC(c2nc(Nc3cnccn3)cc(-c3cccnc3)n2)C1] | C |
| 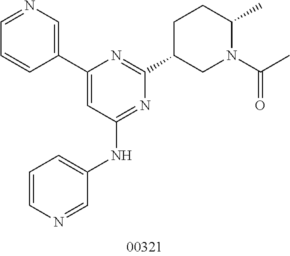 [SMILES: CC(=O)N1C[C@H](c2nc(Nc3cccnc3)cc(-c3cccnc3)n2)CC[C@@H]1C] | A |
| 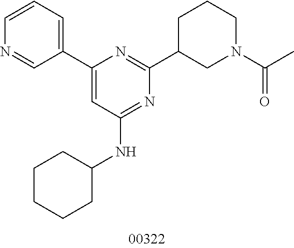 [SMILES: CC(=O)N1CCCC(c2nc(NC3CCCCC3)cc(-c3cccnc3)n2)C1] | C |
| 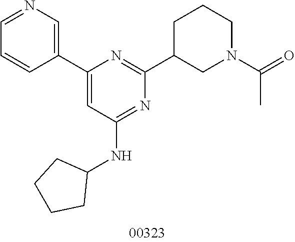 [SMILES: CC(=O)N1CCCC(c2nc(NC3CCCC3)cc(-c3cccnc3)n2)C1] | C |
| 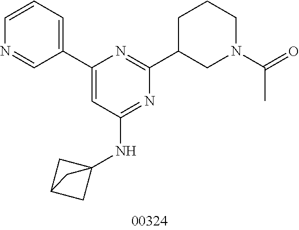 [SMILES: CC(=O)N1CCCC(c2nc(NC34CC(C3)C4)cc(-c3cccnc3)n2)C1] | C |
| 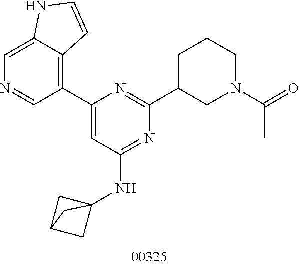 [SMILES: CC(=O)N1CCCC(c2nc(NC34CC(C3)C4)cc(-c3cncc4[nH]ccc34)n2)C1] | B |
| 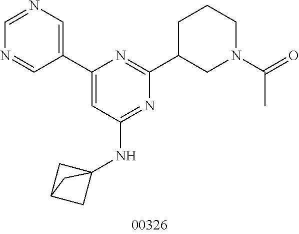 [SMILES: CC(=O)N1CCCC(c2nc(NC34CC(C3)C4)cc(-c3cncnc3)n2)C1] | C |
| 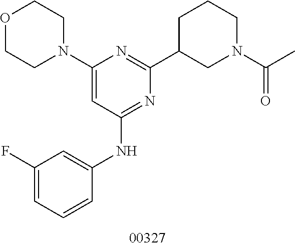 [SMILES: CC(=O)N1CCCC(c2nc(Nc3cccc(F)c3)cc(N3CCOCC3)n2)C1] | C |
| 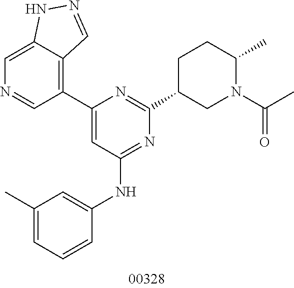 [SMILES: CC(=O)N1C[C@H](c2nc(Nc3cccc(C)c3)cc(-c3cncc4[nH]ncc34)n2)CC[C@@H]1C] | B |
| 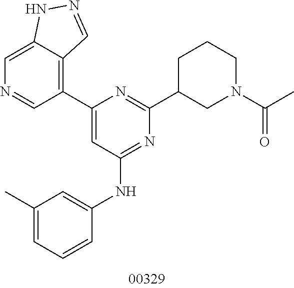 [SMILES: CC(=O)N1CCCC(c2nc(Nc3cccc(C)c3)cc(-c3cncc4[nH]ncc34)n2)C1] | A |
| 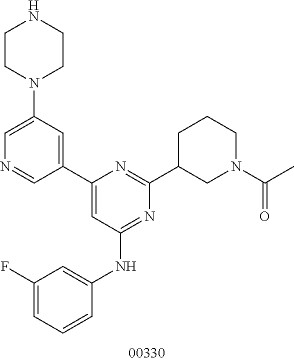 [SMILES: CC(=O)N1CCCC(c2nc(Nc3cccc(F)c3)cc(-c3cncc(N4CCNCC4)c3)n2)C1] | C |
| 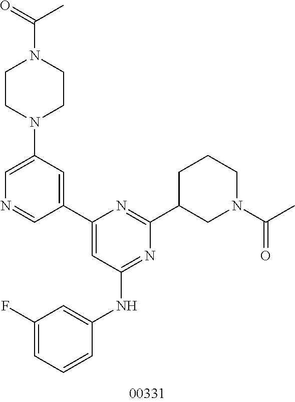 [SMILES: CC(=O)N1CCN(c2cncc(-c3cc(Nc4cccc(F)c4)nc(C4CCCN(C(C)=O)C4)n3)c2)CC1] | B |

Legend EC50: A < 1 μM < B < 10 μM < C

Crystal Structure of the Bromodomain of Human CREBBP in Complex with Compound 00212

Crystallization

Experimental Setup

The construct used for crystallization comprised residues 1081 to 1197. Crystals of CREBBP in complex with compound 00212 were obtained using hanging-drop vapour-diffusion set-ups. CREBBP at a concentration of 20.3 mg/ml (10 mM Hepes, 500 mM NaCl, 5% Glycerol, 0.5 mM TCEP, pH 7.4) was pre-incubated with 4.3 mM (3.0-fold molar excess) of 00212 (150 mM in DMSO) for 1 h. 1 μl of the protein solution was then mixed with 1 μl of reservoir solution (0.1 M MgCl₂, 0.1 M MES/NaOH pH 6.3, 18% (w/v) PEG 6000 and 10% (v/v) ethylene glycol) and equilibrated at 4° C. over 0.4 ml of reservoir solution. Well diffracting crystals appeared and grew to full size over 4 days.

Data Collection

Crystals were cryo-protected by addition of 10% glycerol (final concentration) to the crystallization drop before mounting. A complete 1.6 Å data set of a CREBBP/00212 crystal was collected at Diamond Light Source (Didcot, UK, beamline i03) and the data were integrated, analyzed and scaled by XDS, Pointless and Aimless within the autoPROC pipeline (Table 1).

TABLE 1
Data collection statistics

| Space group | P2₁ |
|---|---|
| Unit cell parameters [Å] | a = 70.4, b = 58.6, c = 73.2 |
| | α = 90.0, β = 115.4, γ = 90.0 |
| Resolution [Å] | 66.14-1.60 (1.63-1.60) |
| # Unique reflections | 68872 (2664) |
| I/σ(I) | 14.9 (2.2) |
| Completeness [%] | 97.2 (75.5) |
| Multiplicity | 3.3 (2.1) |
| Rₘₑₐₛ | 0.050 (0.460) |

Structure Determination and Refinement

Molecular replacement was done using a previously determined structure of CREBBP as a starting model. Several rounds of alternating manual re-building and refinement with REFMAC5 resulted in the final model (Table 2). Atomic displacement factors were modelled with a single isotropic B-factor per atom.

TABLE 2
Refinement statistics

| | |
|---|---|
| Resolution | 35.00-1.60 (1.64-1.60) |
| R_{work} | 0.151 (0.305) |
| R_{free} | 0.190 (0.351) |
| Completeness [%] | 97.2 (77.6) |

Results

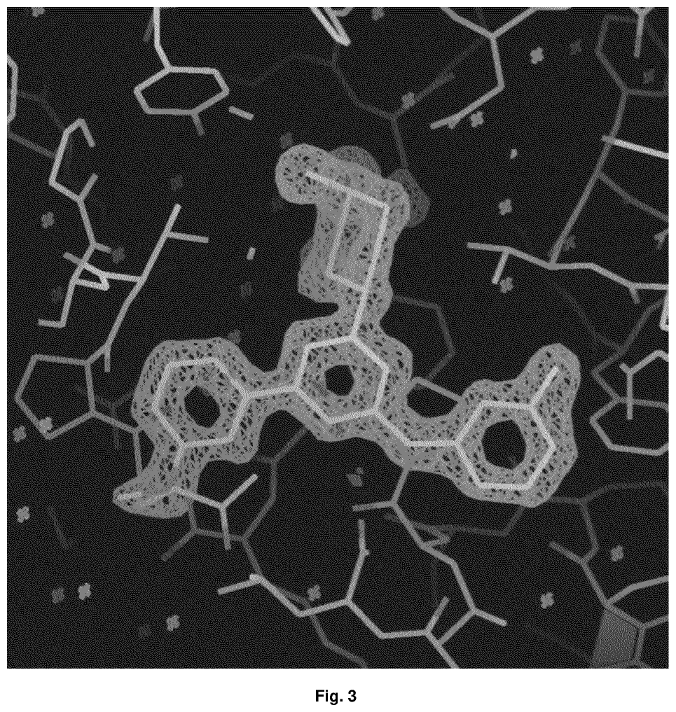

We have produced crystals of CREBBP/00212 that diffracted to 1.6 Å resolution and determined the 3-dimensional structure of the protein-ligand complex. Clear electron density in the Fₒ-F_{c} omit map of the initial model at the compound binding site in each chain of CREBBP revealed the binding of the entire compound (FIG. 3) and allowed its unambiguous placement. Additionally, the structure also confirms the absolute stereochemistry of compound 00212 (2S, 5R on the piperidine moiety).

BromoKdMAX-Assay

A BromoKdMAX was performed at DiscoverX. This assay may be used for determining whether the compounds of the present invention bind to the bromodomain of p300 and/or the bromodomain of CBP with a particular K_{d} (e.g. 100 nM or less).

The assay principle is the following:

BROMOscan™ is a novel industry leading platform for identifying small molecule bromodomain inhibitors. Based on proven KINOMEscan™ technology, BROMOscan™ employs a proprietary ligand binding site-directed competition assay to quantitatively measure interactions between test compounds and bromodomains. This robust and reliable assay panel is suitable for high throughput screening and delivers quantitative ligand binding data to facilitate the identification and optimization of potent and selective small molecule bromodomain inhibitors. BROMOscan™ assays include trace bromodomain concentrations (<0.1 nM) and thereby report true thermodynamic inhibitor Kd values over a broad range of affinities (<0.1 nM to >10 uM).

The assay was conducted as follows:

For the Bromodomain assays, T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

The results were as follows:

| Compound | DiscoveRx | Entrez | | Kd |
|---|---|---|---|---|
| Name | Gene Symbol | Gene Symbol | Modifier | (nM) |
| 00212 | ATAD2A | ATAD2 | > | 10000 |
| 00212 | ATAD2B | ATAD2B | > | 10000 |
| 00212 | BAZ2A | BAZ2A | > | 10000 |
| 00212 | BAZ2B | BAZ2B | > | 10000 |
| 00212 | BRD1 | BRD1 | > | 10000 |
| 00212 | BRD2(1) | BRD2 | > | 10000 |
| 00212 | BRD2(1, 2) | BRD2 | = | 7600 |
| 00212 | BRD2(2) | BRD2 | > | 10000 |
| 00212 | BRD3(1) | BRD3 | > | 10000 |
| 00212 | BRD3(1, 2) | BRD3 | > | 10000 |
| 00212 | BRD3(2) | BRD3 | > | 10000 |
| 00212 | BRD4(1) | BRD4 | > | 10000 |
| 00212 | BRD4(1, 2) | BRD4 | > | 10000 |
| 00212 | BRD4(2) | BRD4 | > | 10000 |
| 00212 | BRD4(full-length, | BRD4 | = | 7100 |
| | short-iso.) | | | |
| 00212 | BRD7 | BRD7 | > | 10000 |
| 00212 | BRD8(1) | BRD8 | > | 10000 |
| 00212 | BRD8(2) | BRD8 | > | 10000 |
| 00212 | BRD9 | BRD9 | > | 10000 |
| 00212 | BRDT(1) | BRDT | > | 10000 |
| 00212 | BRDT(1, 2) | BRDT | > | 10000 |
| 00212 | BRDT(2) | BRDT | > | 10000 |
| 00212 | BRPF1 | BRPF1 | > | 10000 |
| 00212 | BRPF3 | BRPF3 | > | 10000 |
| 00212 | CECR2 | CECR2 | > | 10000 |
| 00212 | CREBBP | CREBBP | = | 29 |
| 00212 | EP300 | EP300 | = | 12 |
| 00212 | FALZ | BPTF | > | 10000 |
| 00212 | GCN5L2 | KAT2A | > | 10000 |
| 00212 | PBRM1(2) | PBRM1 | > | 10000 |
| 00212 | PBRM1(5) | PBRM1 | > | 10000 |
| 00212 | PCAF | KAT2B | > | 10000 |
| 00212 | SMARCA2 | SMARCA2 | > | 10000 |
| 00212 | SMARCA4 | SMARCA4 | > | 10000 |
| 00212 | TAF1(2) | TAF1 | > | 10000 |
| 00212 | TAF1L(2) | TAF1L | > | 10000 |
| 00212 | TRIM24(Bromo.) | TRIM24 | > | 10000 |
| 00212 | TRIM24(PHD, | TRIM24 | > | 10000 |
| | Bromo.) | | | |
| 00212 | TRIM33(PHD, | TRIM33 | > | 10000 |
| | Bromo.) | | | |
| 00212 | WDR9(2) | BRWD1 | > | 10000 |

1. A compound of formula (I), optionally in the form of a pharmaceutically acceptable salt, solvate, cocrystal, tautomer, racemate, enantiomer, or diastereomer or mixture thereof 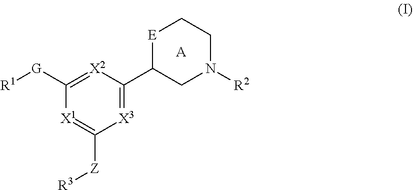
wherein
R¹ is selected from halogen and -(optionally substituted hydrocarbon group which contains from 1 to 20 carbon atoms and optionally 1 to 15 heteroatoms selected from O, N and S);
R² is L-R²¹, wherein L is selected from —C(O)—, —C(O)—O—, —C(O)—NH—; and
R²¹ is selected from hydrogen, -(optionally substituted C₁₋₆ alkyl) which may contain one to three oxygen atoms between carbon atoms, and -(optionally substituted C₃₋₆ cycloalkyl);
R³ is selected from -(optionally substituted heterocyclyl), -(optionally substituted carbocyclyl), -(optionally substituted C₁₋₆ alkylene)-(optionally substituted heterocyclyl) and -(optionally substituted C₁₋₆ alkylene)-(optionally substituted carbocyclyl);
G is selected from a bond, —C(R¹¹)₂—, —N(R¹¹)— and —O—, wherein each R¹¹ is selected from -hydrogen, —C₁₋₆-alkyl, and —(C₁₋₆-alkyl substituted with one or more F); wherein R¹ and any R¹¹ can be optionally linked;
each of X¹, X² and X³ is independently selected from N, CH and CR^{x}, wherein at least one of said X² and X³ is N;
Z is —N(R³¹)—, wherein R³¹ is selected from -hydrogen, —C₁₋₆-alkyl, and —(C₁₋₆-alkyl substituted with one or more F); wherein R³ and any R³¹ can be optionally linked; and
E is either absent or is selected from —CH₂—, —CHR^{x}—, —CR^{x}₂—, —NH—, —NR^{x}—, —O—, -L¹-L²- and -L²-L¹-, wherein L¹ is selected from —CH₂—, —CHR^{x}—, —CR^{x}₂—, —NH—, —NR^{x}— and —O— and L² is selected from —CH₂—, —CHR^{x}— and —CR^{x}₂—;
wherein Ring A may be substituted with one or more groups R^{x}, wherein any two R^{x} groups at ring A can be optionally linked and/or any R^{x} group at ring A can be optionally linked with R²;
wherein Ring A may furthermore be substituted to form a bicyclic moiety having the following partial structure: 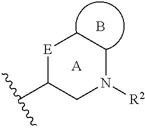
wherein Ring B is an -(optionally substituted heterocycle) or -(optionally substituted carbocycle);
each R^{x} is independently selected from -halogen, —OH, —O-(optionally substituted C₁₋₆ alkyl), —NH-(optionally substituted C₁₋₆ alkyl), —N(optionally substituted C₁₋₆ alkyl)₂, ═O, -(optionally substituted C₁₋₆ alkyl), -(optionally substituted carbocyclyl), -(optionally substituted heterocyclyl), -(optionally substituted C₁₋₆ alkylene)-(optionally substituted carbocyclyl), -(optionally substituted C₁₋₆ alkylene)-(optionally substituted heterocyclyl), —O-(optionally substituted C₁₋₆ alkylene)-(optionally substituted carbocyclyl), and —O-(optionally substituted C₁₋₆ alkylene)-(optionally substituted heterocyclyl), and
wherein the optional substituent of the optionally substituted hydrocarbon group, optionally substituted C₃₋₆ cycloalkyl, optionally substituted heterocyclyl, optionally substituted heterocycle, optionally substituted carbocyclyl, optionally substituted carbocycle and optionally substituted C₁₋₆ alkylene is independently selected from —(C₁₋₆ alkyl which is optionally substituted with one or more halogen), -halogen, —CN, —NO₂, oxo, —C(O)R*, —COOR*, —C(O)NR*R*, —NR*R*, —N(R*)—C(O)R*, —N(R*)—C(O)—OR*, —N(R*)—C(O)—NR*R*, —N(R*)—S(O)₂R*, —OR*, —O—C(O)R*, —O—C(O)—NR*R*, —SR*, —S(O)R*, —S(O)₂R*, —S(O)₂—NR*R*, —N(R*)—S(O)₂—NR*R*, heterocyclyl which is optionally substituted with halogen or C₁₋₆ alkyl, and carbocyclyl which is optionally substituted with halogen or C₁₋₆ alkyl; wherein each R* is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R* connected to the same nitrogen atom can be optionally linked, and
wherein the optional substituent of the optionally substituted C₁₋₆ alkyl and of the optionally substituted C₁₋₆ alkylene is independently selected from -halogen, —CN, —NO₂, OXO, —C(O)R**, —COOR**, —C(O)NR**R**, —NR**R**, —N(R**)—C(O)R**, —N(R**)—C(O)—OR**, —N(R**)—C(O)—NR**R**, —N(R**)—S(O)₂R**, —OR**, —O—C(O)R**, —O—C(O)—NR**R**, —SR**, —S(O)R**, —S(O)₂R**, —S(O)₂—NR**R**, and —N(R**)—S(O)₂—NR**R**; wherein R** is independently selected from H, C₁₋₆ alkyl which is optionally substituted with halogen, heterocyclyl which is optionally substituted with halogen or C₁₋₆ alkyl, and carbocyclyl which is optionally substituted with halogen or C₁₋₆ alkyl; wherein any two R** connected to the same nitrogen atom can be optionally linked.

2. The compound according to claim 1, wherein the compound of formula (I) is a compound of formula (Va) 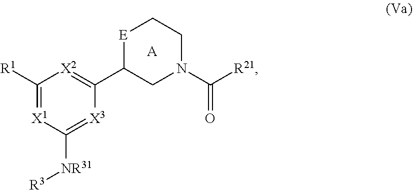
wherein R²¹ is selected from C₁₋₂ alkyl, C₁₋₂ haloalkyl and C₃₋₄ cycloalkyl.

3. The compound according to claim 1 or claim 2, wherein the compound of formula (I) is a compound of formula (Vc)

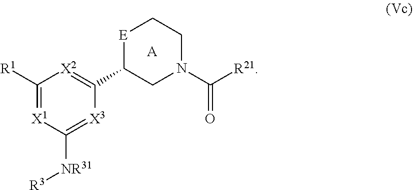

4. The compound according to any one of the preceding claims, wherein the compound of formula (I) is a compound of formula (IVd) 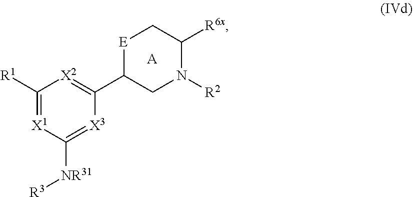
wherein R^{6x} is selected from C₁₋₃ alkyl and C₁₋₂ haloalkyl.

5. The compound according to any one of the preceding claims, wherein R² is —C(O)—R²¹, and wherein R²¹ is —CH₃ or —CH₂CH₃.

6. The compound according to any one of the preceding claims, wherein R² is —C(O)—R²¹, and wherein R²¹ is —CH₃.

7. The compound according to any one of the preceding claims, wherein R³¹ is selected from -hydrogen and —C₁₋₂-alkyl, and wherein preferably R³¹ is -hydrogen.

8. The compound according to any one of the preceding claims, wherein E is selected from —CH₂—, —CHR^{x}—, —NH—, —NR^{x}—, —O—, -L¹-L²- and -L²-L¹-, wherein L¹ is selected from —CH₂—, —CHR^{x}—, —NH—, —NR^{x}— and —O— and L² is selected from —CH₂— and —CHR^{x}—, and preferably wherein E is —CH₂.

9. The compound according to any one of the preceding claims, wherein the number of groups R^{x} in Ring A is 0, 1, or 2.

10. The compound according to any one of the preceding claims, wherein both X² and X³ are nitrogen.

11. The compound according to any one of the preceding claims, wherein X¹ is CH.

12. The compound according to any one of the preceding claims, wherein each R^{x} is independently selected from -halogen, —OH, —O—C₁₋₂ alkyl optionally substituted with one or more R^{xa}, —NH—C₁₋₂ alkyl optionally substituted with one or more R^{xa}, —N(C₁₋₂ alkyl optionally substituted with one or more R^{xa})₂, ═O, C₁₋₃ alkyl optionally substituted with one or more R^{xa}, C₁₋₂ haloalkyl, —W-(monocyclic carbocyclyl optionally substituted with one or more R^{xa}), —W-(monocyclic heterocyclyl optionally substituted with one or more R^{xa}), and wherein —W— is absent, —(C₁₋₂ alkylene)- or —O—(C₁₋₂ alkylene)-, and wherein monocyclic carbocyclyl is selected from phenyl and C₃₋₆ cycloalkyl, and wherein monocyclic heterocyclyl is selected from thiophenyl, pyridyl, pyrazinyl and pyrimidinyl, and wherein said R^{xa} is independently selected from —Cl, —F, and —OH.

13. The compound according to any one of the preceding claims, wherein G is absent and R¹— is selected from -(optionally substituted heterocyclyl) and -(optionally substituted carbocyclyl).

14. The compound according to any one of the preceding claims, wherein G is absent and R¹ is phenyl, azaindolyl, azaindazolyl, pyrazinyl, pyridyl or pyrimidinyl, wherein the phenyl, azaindolyl, azaindazolyl, pyrazinyl, pyridyl or pyrimidinyl is optionally substituted with one or more, preferably one or two, substituents selected from halogen, —C₁₋₆ alkyl, C₁₋₆ haloalkyl, —O—(C₁₋₆ alkyl), —O—(C₁₋₆ haloalkyl), —C(O)—C₁₋₆ alkyl, —C(O)—C₁₋₆ haloalkyl, —NH—C(O)—C₁₋₆ alkyl, —NH—C(O)—C₁₋₆ haloalkyl and —C(O)—NH—C₁₋₆ alkyl, —C(O)—NH—C₁₋₆ haloalkyl.

15. The compound according to any one of the preceding claims, wherein R³ is phenyl or pyridyl, each of which is optionally substituted with one or more groups selected from halogen, —(C₁₋₆ alkyl which is optionally substituted with one or more F) and —O—(C₁₋₆ alkyl which is optionally substituted with one or more F).

16. The compound according to any one of the preceding claims, wherein the compound of formula (I) binds to the bromodomain of p300 and/or the bromodomain of CBP with an EC50 of 10000 nM or less.

17. A pharmaceutical composition comprising:
a compound having the formula (I) as defined in any of claims 1 to 16, optionally in the form of a pharmaceutically acceptable salt, solvate, cocrystal, tautomer, racemate, enantiomer, or diastereomer or mixture thereof,
and optionally one or more pharmaceutically acceptable excipient(s) and/or carrier(s).

18. A compound having the formula (I) as defined in any of claims 1 to 16, optionally in the form of a pharmaceutically acceptable salt, solvate, cocrystal, tautomer, racemate, enantiomer, or diastereomer or mixture thereof, or the pharmaceutical composition of claim 17, wherein the compound or pharmaceutical composition is for use in the treatment, amelioration or prevention of cancer.

19. A method of treating, ameliorating or preventing cancer, the method comprising administering to a patient in need thereof a therapeutically effective amount of a compound having the formula (I) as defined in any of claims 1 to 16, optionally in the form of a pharmaceutically acceptable salt, solvate, cocrystal, tautomer, racemate, enantiomer, or diastereomer or mixture thereof, or a pharmaceutical composition of claim 17.

20. A compound for use according to claim 18, a pharmaceutical composition for use according to claim 18 or the method according to claim 19, wherein the cancer is selected from melanoma, non-small cell lung cancer, prostate cancer, bile duct cancer, bladder cancer, pancreatic cancer, thyroid cancer, ovarian cancer, colorectal tumor, hairy cell leukemia, acute myeloid leukemia, multiple myeloma, liver cancer, breast cancer, esophageal cancer, head and neck cancer and glioma, in particular melanoma and non-small cell lung cancer.

21. A method of treating, ameliorating or preventing cancer, the method comprising administering to a patient in need thereof a therapeutically effective amount of a compound having the formula (I) as defined in any of claims 1 to 16, optionally in the form of a pharmaceutically acceptable salt, solvate, cocrystal, tautomer, racemate, enantiomer, or diastereomer or mixture thereof, or a pharmaceutical composition of claim 17, wherein the method further comprises administering to the patient in need thereof a second therapeutic agent.

22. The method of claim 21, wherein the second therapeutic agent is an immune-oncology agent.
